(19) United States
(12) Patent Application Publication
(10) Pub. No.: US 2026/0166181 A1
(43) Pub. Date: Jun. 18, 2026

(54) COMPOSITIONS AND METHODS FOR TREATING AMYOTROPHIC LATERAL SCLEROSIS AND DISORDERS ASSOCIATED WITH THE SPINAL CORD

(71) Applicant: Voyager Therapeutics, Inc., Lexington, MA (US)

(72) Inventors: Mathieu Emmanuel NONNENMACHER, Boston, MA (US); Mathew Alan CHILD, Ashland, MA (US); Jinzhao HOU, Lexington, MA (US); Jiangyu LI, Watertown, MA (US); Shaoyong LI, Hopkinton, MA (US); Tyler Christopher MOYER, Boston, MA (US); Wei WANG, Arlington, MA (US); Cansu COLPAN, North Billerica, MA (US); Michael D. GRANNAN, Medford, MA (US); Brett HOFFMAN, Braintree, MA (US); Elisabeth KNOLL, Melrose, MA (US); Yanqun SHU, Winchester, MA (US); Nilesh Navalkishor PANDE, North Grafton, MA (US); Jeffrey Scott THOMPSON, Stoneham, MA (US)

(21) Appl. No.: 18/711,046

(22) PCT Filed: Nov. 17, 2022
(86) PCT No.: PCT/US2022/080035

§ 371 Date: May 16, 2024

Related U.S. Application Data
(60) Provisional application No. 63/280,582, filed on Nov. 17, 2021.
(60) Provisional application No. 63/341,274, filed on May 12, 2022.

(51) Int. Cl. A61K 48/00; C12N 15/113; C12N 15/86

(52) U.S. Cl. CPC A61K 48/0066; C12N 15/1137; C12N 15/86; C12N 2750/14143; C12N 2750/14152

(57) ABSTRACT

The present disclosure relates to AAVs encoding a SOD1 targeting polynucleotide which may be used to treat amyotrophic lateral sclerosis (ALS) and delivery methods for the treatment of spinal cord related disorders including ALS.

### RELATED APPLICATION

This application claims priority to U.S. Provisional Application No. 63/280,582 filed on Nov. 17, 2021, and to U.S. Provisional Application No. 63/341,274 filed on May 12, 2022; the entire contents each of which are hereby incorporated by reference in their entirety.

### FIELD OF THE DISCLOSURE

The present disclosure relates to compositions, preparation, use, and/or formulation of adeno-associated virus (AAV) capsid proteins and variants thereof, for the delivery of a SOD1 targeting polynucleotide, e.g., small interfering RNA (siRNA) duplexes, shRNA, microRNA (miRNA), or precursors thereof which target or encode molecules which target the superoxide dismutase 1 (SOD1) gene to interfere with SOD1 gene expression and/or SOD1 enzyme production. Methods for inhibiting SOD1 or altering the expression of any gene associated with a spinal cord related disease or disorder in a subject with a disease and/or other disorder associated with the spinal cord are also disclosed. The method includes the administration of the at least one polynucleotide into the subject with a disorder associated with the spinal cord (e.g., neurodegenerative disease) via at least intravenous injection or via intra-cisterna magna injection. In these embodiments the disease is a motor neuron disease, and more specifically, the disease is amyotrophic lateral sclerosis (ALS).

### BACKGROUND

Amyotrophic lateral sclerosis (ALS), also known as Lou Gehrig's disease, is a fatal progressive neurodegenerative disease, characterized by the predominant loss of upper and lower motor neurons (MNs) in primary motor cortex, the brainstem, and the spinal cord. Upper (e.g., cortical) and lower motor neurons (e.g., spinal cord) normally communicate messages from the brain to the muscles to generate voluntary movement. When these neurons degenerate and/or die, the loss of the message to the muscles results in a gradual weakening and/or atrophy of the muscle and inability to initiate or control voluntary movements, until ultimately, an individual suffering from ALS loses muscle strength and the ability to move, speak, eat and even breathe. Most patients will require some form of breathing aid for survival, and even then, most ALS patients die as a result of respiratory failure within 2-5 years of diagnosis. During disease progression, some patients (e.g., FTD-ALS) may also develop frontotemporal dementia.

Two forms of ALS have been described: one is sporadic ALS (sALS), which is the most common form of ALS in the United States of America and accounts for 90 to 95% of all cases diagnosed; the other is familial ALS (fALS), which occurs in a family lineage mainly with a dominant inheritance and only accounts for about 5 to 10% of all cases in the United States of America. sALS and fALS are clinically indistinguishable.

Pathological studies have linked numerous cellular processes with disease pathogenesis such as increased ER stress, generation of free radicals (i.e., reactive oxygen species (ROS)), mitochondrial dysfunction, protein aggregation, apoptosis, inflammation and glutamate excitotoxicity, specifically in the motor neurons (MNs).

The causes of ALS are complicated and heterogeneous. In general, ALS is considered to be a complex genetic disorder in which multiple genes in combination with environmental exposures combine to render a person susceptible. More than a dozen genes associated with ALS have been discovered, including, SOD1 (Cu²⁺/Zn²⁺ superoxide dismutase), TDP-43 (TARDBP, TAR DNA binding protein-43), FUS (Fused in Sarcoma/Translocated in Sarcoma), ANG (Angiogenin), ATXN2 (Ataxin-2), valosin containing protein (VCP), OPTN (Optineurin) and an expansion of the noncoding GGGGCC hexanucleotide repeat in the chromosome 9, open reading frame 72 (C90RF72). However, the exact mechanisms of motor neuron degeneration are still elusive.

Currently, there is no curative treatment for ALS.

SOD1 can be a potential therapeutic target for both familial and sporadic ALS. A therapy that can reduce the SOD1 protein, whether wildtype or mutant, produced in the central nervous system of ALS patients may ameliorate the symptoms of ALS in patients such as motor neuron degeneration and muscle weakness and atrophy. Agents and methods that aim to prevent the formation of wild type and/or mutant SOD1 protein aggregation may prevent disease progression and allow for amelioration of ALS symptoms. RNA interfering (RNAi) mediated gene silencing has drawn researchers' interest in recent years. Small double stranded RNA (small interfering RNA) molecules that target the SOD1 gene have been taught in the art for their potential in treating ALS (See, e.g., U.S. Pat. No. 7,632,938 and U.S. Patent Publication No. 20060229268).

### SUMMARY

The present disclosure provides AAV particles encoding a SOD1 targeting polynucleotide to interfere with SOD1 gene expression and/or SOD1 protein production and methods of use thereof. Methods for treating diseases associated with motor neuron degeneration such as amyotrophic lateral sclerosis are also included in the present disclosure.

In one aspect, the present disclosure provides an isolated, e.g., recombinant, AAV particle comprising an AAV capsid variant and a nucleic acid comprising a transgene encoding SOD1 targeting polynucleotide (e.g., a SOD1 targeting RNA agent, e.g., a siRNA duplex), wherein the AAV capsid variant: (i) is enriched at least about 5, 6, 7, 8, 9, 10, 20, 30, 40, 50, 100, 200, 300, or 400-fold, in the brain, e.g., the brain of a non-human primate (NHP) compared to a reference sequence of SEQ ID NO: 138 (e.g., as provided in Table 20), e.g., when measured by an assay as described in Example 2; (ii) transduces a brain region, e.g., a brain region of an NHP, e.g., selected from dentate nucleus, cerebellar cortex, cerebral cortex, brain stem, hippocampus, thalamus and putamen, wherein the level of transduction is at least 5, 10, 50, 100, 200, 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 3; (iii) delivers an increased level of a payload to a brain region, e.g., a brain region of an NHP, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus; (iv) delivers an increased level of a payload to a spinal cord region, e.g., a spinal cord region of an NHP, optionally wherein the level of the payload is increased by at least 10, 20, 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 3), optionally wherein the spinal cord region comprises a cervical, thoracic, and/or lumbar region; and/or (v) delivers an increased level of viral genomes to a brain region, e.g., a brain region of an NHP, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus.

In another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises: (a) the amino acid sequence of any of SEQ ID NO: 3648-3659 or a sequence provided in Table 20; (b) an amino acid sequence comprising at least 5, 6, 7, 8, or 9 consecutive amino acids from the amino acid sequence of any of SEQ ID NO: 3648-3659 or a sequence provided in Table 20; (c) an amino acid sequence comprising at least one, two, or three but no more than four modifications, e.g., substitutions, (e.g., conservative substitutions), insertions, or deletions, relative to the amino acid sequence of any of 3648-3659 or a sequence provided in Table 20; or (d) an amino acid sequence comprising at least one, two, or three but no more than four different amino acids relative to the amino acid sequence of any of 3648-3659 or a sequence provided in Table 20; optionally wherein the capsid variant comprises the amino acid sequence of SEQ ID NO: 138, or an amino acid sequence with at least 95% sequence identity thereto.

In a further aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises: (i) the amino acid sequence of PLNGAVHLY (SEQ ID NO: 3648); (ii) an amino acid sequence comprising at least 5, 6, 7, 8, or 9 consecutive amino acids from the amino acid sequence of PLNGAVHLY (SEQ ID NO: 3648); (iii) an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648); or (iv) an amino acid sequence comprising at least one, two, or three but no more than four different amino acids relative to the amino acid sequence of PLNGAVHLY (SEQ ID NO: 3648); optionally wherein the capsid variant comprises the amino acid sequence of SEQ ID NO: 138, or an amino acid sequence with at least 95% sequence identity thereto.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises an amino sequence comprising the following formula: [N1]-[N2], wherein: (i) [N1] comprises X1, X2, X3, X4, and X5, wherein: (a) position X1 is: P, Q, A, H, K, L, R, S, or T; (b) position X2 is: L, I, V, H, or R; (c) position X3 is: N, D, I, K, or Y; (d) position X4 is: G, A, C, R, or S; and (e) position X5 is: A, S, T, G, C, D, N, Q, V, or Y; and (ii) [N2] comprises the amino acid sequence of VHLY (SEQ ID NO: 3741), VHIY (SEQ ID NO: 3746), VHVY (SEQ ID NO: 3742), or VHHY (SEQ ID NO: 3747); and/or an amino acid modification, e.g., a conservative substitution, of any of the aforesaid amino acids in (i) and/or (ii).

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises one, two, three, four, or all of: (i) an [N1], wherein [N1] is or comprises: SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), PLDSS (SEQ ID NO: 3723), PLNGC (SEQ ID NO: 3724), PLYGA (SEQ ID NO: 3725), TLNGA (SEQ ID NO: 3726), PVDGA (SEQ ID NO: 3727), PLKGA (SEQ ID NO: 3728), PLNGD (SEQ ID NO: 3729), KLDGA (SEQ ID NO: 3730), PHNGA (SEQ ID NO: 3731), PLNGV (SEQ ID NO: 3732), PLNAA (SEQ ID NO: 3733), QLNGY (SEQ ID NO: 3734), PLDGS (SEQ ID NO: 3735), LLNGA (SEQ ID NO: 3736), PLNRA (SEQ ID NO: 3737), PLIGA (SEQ ID NO: 3738), PRNGA (SEQ ID NO: 3739), or ALNGS (SEQ ID NO: 3740); (ii) an [N2] wherein [N2] is or comprises: VHLY (SEQ ID NO: 3741), VHVY (SEQ ID NO: 3742), VPLY (SEQ ID NO: 3743), VNLY (SEQ ID NO: 3744), VHRY (SEQ ID NO: 3745), VHIY (SEQ ID NO: 3746), VHHY (SEQ ID NO: 3747), FHLY (SEQ ID NO: 3748), LHLY (SEQ ID NO: 3749), DHLY (SEQ ID NO: 3750), VQLY (SEQ ID NO: 3751), IHLY (SEQ ID NO: 3752), VDLY (SEQ ID NO: 3753), AHLY (SEQ ID NO: 3754), VLLY (SEQ ID NO: 3755), GHLY (SEQ ID NO: 3756), VRLY (SEQ ID NO: 3757), or VYLY (SEQ ID NO: 3758); (iii) an [N3] wherein [N3] is or comprises: AQAQ (SEQ ID NO: 3759), SQAQ (SEQ ID NO: 3760), AQPQ (SEQ ID NO: 3761), AQSQ (SEQ ID NO: 3762), AKAQ (SEQ ID NO: 3763), AHAQ (SEQ ID NO: 3764), AQAP (SEQ ID NO: 3765), DQAQ (SEQ ID NO: 3766), APAQ (SEQ ID NO: 3767), AQAK (SEQ ID NO: 3768), AQAH (SEQ ID NO: 3769), AQEQ (SEQ ID NO: 3770), ALAQ (SEQ ID NO: 3771), ARAQ SEQ ID NO: 3772), or TQAQ (SEQ ID NO: 3773); (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or wherein the AAV capsid variant comprises an amino acid modification, e.g., a conservative substitution, of any of the aforesaid amino acids in (i)-(v).

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises one, two, three, four, or all of: (i) an [N1], wherein [N1] is or comprises: SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), or PLDSS (SEQ ID NO: 3723); (ii) an [N2] wherein [N2] is or comprises: VHLY (SEQ ID NO: 3741) or VHVY (SEQ ID NO: 3742); (iii) an [N3] wherein [N3] is or comprises: AQAQ (SEQ ID NO: 3759), SQAQ (SEQ ID NO: 3760), AQPQ (SEQ ID NO: 3761), or AQSQ (SEQ ID NO: 3762); (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or (v) an [N5] wherein [N5] is or comprises: VQN, VKN, VQT, VQK, DQN, VQH, GQN, VQI, VHN, FQN, LQN, VLN, VRN, VQS, VQY, AQN, VEN, VQD; and/or wherein the AAV capsid variant comprises an amino acid modification, e.g., a conservative substitution, of any of the aforesaid amino acids in (i)-(v).

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises [A][B], wherein [A] comprises the amino acid sequence of PLNGA (SEQ ID NO: 3679), and [B] comprises X1, X2, X3, X4, wherein: (i) X1 is: V, I, L, A, F, D, or G; (ii) X2 is: H, N, Q, P, D, L, R, or Y; (iii) X3 is: L, H, I, R, or V; and (iv) X4 is Y; and/or wherein the AAV capsid variant comprises an amino acid modification, e.g., a conservative substitution, of any of the aforesaid amino acids in (i)-(iv); optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises an amino sequence comprising the amino acid sequence of PLNGAVHLY (SEQ ID NO: 3648); and which further comprises one, two, three, or all of: (i) the amino acid at position 593, relative to a reference sequence numbered according to the amino acid sequence of SEQ ID NO: 138, is: T, A, L, R, V, C, I, K, M, N, P, Q, or S; (ii) the amino acid at position 594, relative to a reference sequence numbered according to the amino acid sequence of SEQ ID NO: 138, is: G, M, S, A, Q, V, T, L, P, H, K, N, I, Y, or R; and/or (iii) the amino acid at position 595, relative to a reference sequence numbered according to the amino acid sequence of SEQ ID NO: 138, is: W, S, P, T, A, G, L, Q, H, N, R, K, V, E, F, M, C, or Y; optionally, provided that the amino acids at positions 593-595, relative to a reference sequence numbered according to the amino acid sequence of SEQ ID NO: 138, does not comprise the amino acid sequence of TGW.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises X1-X2-X3-X4-X5-X6-X7-X8-X9-X10-X11-X12-X13-X14-X15-X16-X17-X18-X19, wherein: (i) X1 is: P, A, D, E, F, G, H, K, L, N, Q, R, S, T, or V; (ii) X2 is independently chosen from: L, D, E, F, H, I, M, N, P, Q, R, S, or V; (iii) X3 is: N, A, D, E, G, H, I, K, Q, S, T, V, or Y; (iv) X4 is: G, A, C, D, E, P, Q, R, S, T, V, or W; (v) X5 is: A, C, D, E, F, G, H, I, K, N, P, Q, R, S, T, V, W, or Y; (vi) X6 is: V, A, C, D, E, F, G, H, I, K, L, M, N, Q, R, S, T, or Y; (vii) X7 is: H, A, D, E, G, I, K, L, M, N, P, Q, R, S, T, V, or Y; (viii) X8 is independently chosen from: L, A, D, E, F, G, H, I, K, M, N, P, Q, R, S, T, V, or Y; (ix) X9 is independently chosen from: Y, A, C, D, E, F, G, H, I, K, L, M, N, Q, R, S, T, V, or W; (x) X10 is independently chosen from: A, C, D, E, F, G, H, I, K, L, N, P, Q, R, S, T, V, or; Y; (xi) X11 is independently chosen from: Q, A, D, E, H, K, L, P, R, or T; (xii) X12 is: A, D, E, G, H, L, N, P, Q, R, S, T, or V; (xiii) X13 is: Q, E, H, K, L, P, R, or T; (xiv) X14 is: T, A, C, D, E, F, G, H, I, K, L, M, N, P, Q, R, S, V, W, or Y; (xv) X15 is: G, A, C, D, E, F, H, I, K, L, M, N, P, Q, R, S, T, V, W, or Y; (xvi) X16 is: W, A, C, D, E, F, G, H, I, K, L, M, N, P, Q, R, S, T, V, or Y; (xvii) X17 is: V, A, D, E, F, G, H, I, or L; (xviii) X18 is: Q, E, H, K, L, P, or R; and/or (xix) X19 is: N, D, H, I, K, P, S, T, or Y.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises: (a) the amino acid sequence of any one of SEQ ID NOs: 139-1138; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or (c) an amino acid sequence comprising at least one, two, or three but no more than four different amino acids, relative to the amino acid sequence of any one of SEQ ID NOs: 139-1138; (d) an amino sequence comprising at least one, two, or three but no more than four modifications, e.g., substitutions (e.g., conservative substitutions), insertions, or deletions, relative to the amino acid sequence of any one of SEQ ID NOs: 139-1138; optionally wherein the AAV capsid variant does not comprise the amino acid sequence of TGW at positions 593-595, relative to a reference sequence numbered according to the amino acid sequence of SEQ ID NO: 138.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), wherein the AAV capsid variant comprises: (a) the amino acid sequence of any one of SEQ ID NOs: 139-476; (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or (c) an amino acid sequence comprising at least one, two, or three but no more than four different amino acids, relative to the amino acid sequence of any one of SEQ ID NOs: 139-476; (d) an amino sequence comprising at least one, two, or three but no more than four modifications, e.g., substitutions (e.g., conservative substitutions), insertions, or deletions, relative to the amino acid sequence of any one of SEQ ID NOs: 139-476; optionally wherein the AAV capsid variant does not comprise the amino acid sequence of TGW at positions 593-595, relative to a reference sequence numbered according to the amino acid sequence of SEQ ID NO: 138.

In yet another aspect, the present disclosure provides an isolated, e.g., recombinant, AAV particle comprising an AAV capsid variant and a nucleic acid comprising a transgene encoding a SOD1 targeting polynucleotide (e.g., a SOD1 targeting RNA agent), or a fragment thereof, wherein the AAV capsid variant comprises: (a) the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113; (b) an amino acid sequence comprising at least 3, 4, 5, 6, 7, 8, 9, 10, 11, 12, 13, 14, 15, 16, 17, or 18 consecutive amino acids from any one of the amino acid sequences in (i); or (c) an amino acid sequence comprising at least one, two, or three but no more than four different amino acids, relative to the amino acid sequence of any one of the amino acid sequences in (i); (d) an amino sequence comprising at least one, two, or three but no more than four modifications, e.g., substitutions (e.g., conservative substitutions), insertions, or deletions, relative to the amino acid sequence of any one of the amino acid sequences in (i); optionally wherein the AAV capsid variant does not comprise the amino acid sequence of TGW at positions 593-595, relative to a reference sequence numbered according to the amino acid sequence of SEQ ID NO: 138.

In accordance with some aspects of the present disclosure the SOD1 targeting polynucleotide, e.g., the SOD1 targeting RNA agent, is a single-stranded antisense RNA molecule, a single-stranded siRNA, or a double-stranded RNA (e.g., a siRNA duplex) that inhibits expression of SOD1. For example, the SOD1 targeting polynucleotide may be a siRNA duplex comprising: (i) a sense strand sequence comprising at least 15 (e.g., at least 16, 17, 18, 19, 20, 21, 22, or 23) contiguous nucleotides differing by no more than 3 (e.g., by no more than 0, 1 or 2) nucleotides from a sense sequence listed in Table 10; and (ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from an antisense sequence in Table 10.

In yet another aspect, the present disclosure provides a cell, e.g., a host cell, comprising the AAV particles described herein. The method of making the AAV particle may comprise: (i) providing a host cell comprising a viral genome; and (ii) incubating the host cell under conditions suitable to enclose the viral genome in an AAV capsid variant, e.g., an AAV capsid variant described herein; thereby making the AAV particle.

Further disclosed are pharmaceutical compositions including the AAV particles as described herein, and a pharmaceutically acceptable excipient. The administration of the pharmaceutical compositions may be used in the methods of treating a subject having or diagnosed with having a neurological disorder, e.g., a neurodegenerative disorder. In some embodiments the neurological disorder, neurodegenerative disorder, or disease associated with SOD1 expression is amyotrophic lateral sclerosis (ALS). In some embodiments, the AAV particles are administered intravenously or by ICM, or a combination thereof.

In some aspects, ALS is familial ALS linked to SOD1 mutations. In other aspects, ALS is sporadic ALS which is characterized by abnormal aggregation of SOD1 protein or disruption of SOD1 protein function or localization, though not necessarily as a result of genetic mutation. The symptoms of ALS ameliorated by the present method may include motor neuron degeneration, muscle weakness, stiffness of muscles, slurred speech and/or difficulty in breathing. The ALS may be early stage ALS, middle stage ALS; and/or late stage ALS

In some embodiments, the pharmaceutical composition of the present disclosure is used as a solo therapy. In other embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

### Enumerated Embodiments

1. An isolated, e.g., recombinant, AAV particle comprising an AAV capsid variant and a nucleic acid comprising a transgene encoding SOD1 targeting polynucleotide (e.g., a SOD1 targeting RNA agent, e.g., a siRNA duplex), wherein the AAV capsid variant:
   (i) is enriched at least about 5, 6, 7, 8, 9, 10, 20, 30, 40, 50, 100, 200, 300, or 400-fold, in the brain, e.g., the brain of a non-human primate (NHP) compared to a reference sequence of SEQ ID NO: 138 (e.g., as provided in Table 20), e.g., when measured by an assay as described in Example 2;
   (ii) transduces a brain region, e.g., a brain region of an NHP, e.g., selected from dentate nucleus, cerebellar cortex, cerebral cortex, brain stem, hippocampus, thalamus and putamen, wherein the level of transduction is at least 5, 10, 50, 100, 200, 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 3;
   (iii) delivers an increased level of a payload to a brain region, e.g., a brain region of an NHP, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus;
   (iv) delivers an increased level of a payload to a spinal cord region, e.g., a spinal cord region of an NHP, optionally wherein the level of the payload is increased by at least 10, 20, 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 3), optionally wherein the spinal cord region comprises a cervical, thoracic, and/or lumbar region; and/or
   (v) delivers an increased level of viral genomes to a brain region, e.g., a brain region of an NHP, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus.
2. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises:
   (a) the amino acid sequence of any of SEQ ID NO: 3648-3659 or a sequence provided in Table 20;
   (b) an amino acid sequence comprising at least 5, 6, 7, 8, or 9 consecutive amino acids from the amino acid sequence of any of SEQ ID NO: 3648-3659 or a sequence provided in Table 20;
   (c) an amino acid sequence comprising at least one, two, or three but no more than four modifications, e.g., substitutions, (e.g., conservative substitutions), insertions, or deletions, relative to the amino acid sequence of any of 3648-3659 or a sequence provided in Table 20; or
   (d) an amino acid sequence comprising at least one, two, or three but no more than four different amino acids relative to the amino acid sequence of any of 3648-3659 or a sequence provided in Table 20;
   optionally wherein the capsid variant comprises the amino acid sequence of SEQ ID NO: 138, or an amino acid sequence with at least 95% sequence identity thereto.
3. The isolated AAV particle of any one of embodiments 1 or 2, wherein the AAV capsid variant comprises:
   (a) the amino acid sequence of PLNGAVHLY (SEQ ID NO: 3648), or at least 5, 6, 7, or 8 consecutive amino acids from the amino acid sequence of PLNGAVHLY (SEQ ID NO: 3648);
   (b) the amino acid sequence of IVMNSLK (SEQ ID NO: 3651), or at least 4, 5, or 6 consecutive amino acids from the amino acid sequence of IVMNSLK (SEQ ID NO: 3651); or
   (c) the amino acid sequence of YSTDVRM (SEQ ID NO: 3650), YSTDERM (SEQ ID NO: 3657), or YSTDERK (SEQ ID NO: 3658), or at least 4, 5, or 6 consecutive amino acids from the amino acid sequence of YSTDVRM (SEQ ID NO: 3650), YSTDERM (SEQ ID NO: 3657), or YSTDERK (SEQ ID NO: 3658).
4. The isolated AAV particle of embodiment 2 or 3, wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); and/or
   (v) the 9 consecutive amino acids comprise PLNGAVHLY (SEQ ID NO: 3648);
   optionally wherein the amino acid sequence of (i), (ii), (iii), (iv), or (v) is present immediately subsequent to position 586, relative to a reference sequence numbered according to the amino acid sequence of SEQ ID NO: 138.
5. The isolated AAV particle of any one of embodiments 2-4, wherein:
   (i) the 5 consecutive amino acids comprise IVMNS (SEQ ID NO: 3694);
   (ii) the 6 consecutive amino acids comprise IVMNSL (SEQ ID NO: 3695); and/or
   (iii) the 7 consecutive amino acids comprise IVMNSLK (SEQ ID NO: 3651),
   optionally wherein the amino acid sequence of (i), (ii), or (iii) is present immediately subsequent to position 588, relative to a reference sequence numbered according to the amino acid sequence of SEQ ID NO: 138.
6. The isolated AAV particle of any one of embodiments 1-5, wherein the AAV capsid variant comprises:
   (i) the amino acid sequence of PLNGAVHLY (SEQ ID NO: 3648), wherein the amino acid sequence of PLNGAVHLY (SEQ ID NO: 3648) is present immediately subsequent to position 586, relative to a reference sequence numbered according to the amino acid sequence of SEQ ID NO: 138;
   (ii) the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138;
   (iii) the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138; or
   (iv) the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.
7. The isolated AAV particle of any one of embodiments 1-6, wherein:
   (i) the AAV capsid variant comprises an amino acid sequence encoded by the nucleotide sequence of any one of SEQ ID NOs: 3660-3671, or a nucleotide sequence having at least 80%, 85%, 90%, 92%, 95%, 96%, 97%, 98%, or 99% sequence identity thereto;
   (ii) the AAV capsid variant comprises an amino acid sequence encoded by a nucleotide sequence comprising at least one, two, three, four, five, six, or seven modifications, e.g., substitutions (e.g., conservative substitutions), but no more than ten modifications of the nucleotide sequences of any of SEQ ID NOs: 3660-3671;
   (iii) the AAV capsid variant comprises an amino acid sequence encoded by a nucleotide sequence comprising at least one, two, three, four, five, six, or seven but no more than ten different nucleotides relative to the nucleotide sequences of any of SEQ ID NOs: 3660-3671;
   (iv) the nucleotide sequence encoding the AAV capsid variant comprises the nucleotide sequence of any one of SEQ ID NOs: 3660-3671, or a nucleotide sequence having at least 80%, 85%, 90%, 92%, 95%, 96%, 97%, 98%, or 99% sequence identity thereto; and/or
   (v) the nucleotide sequence encoding the AAV capsid variant comprises a nucleotide sequence comprising at least one, two, three, four, five, six, or seven modifications, e.g., substitutions (e.g., conservative substitutions), but no more than ten modifications of the nucleotide sequence of any of SEQ ID NOs: 3660-3671.
8. The isolated AAV particle of any one of embodiments 1-5, wherein the AAV capsid variant comprises:
   (i) the AAV capsid variant comprises the amino acid sequence of any one of SEQ ID NOs: 3636-3647, or an amino acid sequence with at least 90% (e.g., at least 95, 96, 97, 98, or 99%) sequence identity thereto; or
   (ii) the nucleotide sequence encoding the AAV capsid variant comprises the nucleotide sequence of any one of SEQ ID NOs: 3623-3635, or a nucleotide sequence with at least 90% (e.g., at least 95, 96, 97, 98, or 99%) sequence identity thereto.
9. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid sequence of PLNGAVHLY (SEQ ID NO: 3648);
   (ii) an amino acid sequence comprising at least 5, 6, 7, 8, or 9 consecutive amino acids from the amino acid sequence of PLNGAVHLY (SEQ ID NO: 3648);
   (iii) an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648); or
   (iv) an amino acid sequence comprising at least one, two, or three but no more than four different amino acids relative to the amino acid sequence of PLNGAVHLY (SEQ ID NO: 3648);
   optionally wherein the capsid variant comprises the amino acid sequence of SEQ ID NO: 138, or an amino acid sequence with at least 95% sequence identity thereto.
10. The isolated AAV particle of embodiment 9, wherein the amino acid sequence of (i), (ii), or (iii) is present in loop VIII, relative to a reference sequence of SEQ ID NO: 138.
11. The isolated AAV particle of any one of embodiments 9 or 10, wherein the amino acid sequence is present immediately subsequent to position 586, relative to a reference sequence numbered according to the amino acid sequence of SEQ ID NO: 138.
12. The isolated AAV particle of any one of embodiments 1-11, wherein the AAV capsid variant:
   (i) is enriched at least about 300 or 400-fold compared, in the brain compared to a reference sequence of SEQ ID NO: 138, e.g., when measured by an assay as described in f;
   (ii) transduces a brain region, e.g., selected from dentate nucleus, cerebellar cortex, cerebral cortex, brain stem, hippocampus, thalamus and putamen, wherein the level of transduction is at least 500, 1,000, 2,000, 5,000, or 10,000-fold greater as compared to a reference sequence of SEQ ID NO: 138, e.g., when measured by an assay, e.g., an immunohistochemistry assay, a qRT-PCR, or a RT-ddPCR assay, e.g., as described in Example 3;
   (iii) delivers an increased level of a payload to a brain region, optionally wherein the level of the payload is increased by at least 500, 1,000, 2,000, 5,000, or 10,000-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus;
   (iv) delivers an increased level of a payload to a spinal cord region, optionally wherein the level of the payload is increased by at least 50, 100, 200, 300, 400, 500, 600, 700, 800 or 900-fold, as compared to a reference sequence of SEQ ID NO: 138, e.g., when measured by an assay, e.g., a qRT-PCR assay (e.g., as described in Example 3), optionally wherein the spinal cord region comprises a cervical, thoracic, and/or lumbar region; and/or
   (v) delivers an increased level of viral genomes to a brain region, optionally wherein the level of viral genomes is increased by at least 5, 10, 20, 30, 40 or 50-fold, as compared to a reference sequence of SEQ ID NO: 138, e.g., when measured by an assay, e.g., a qRT-PCR or a RT-ddPCR assay (e.g., as described in Example 3), optionally wherein the brain region comprises a frontal cortex, sensory cortex, motor cortex, putamen, thalamus, cerebellar cortex, dentate nucleus, caudate, and/or hippocampus.
13. The isolated AAV particle of any one of embodiments 1-8 or 10, wherein:
   (i) the AAV capsid variant has an increased tropism for a muscle cell or tissue, e.g., a heart tissue, relative to the tropism of a reference sequence comprising the amino acid sequence of SEQ ID NO: 138; and/or
   (ii) the AAV capsid variant delivers an increased level of a payload to a muscle region, optionally wherein the level of the payload is increased by at least 10, 15, 20, 30, or 40-fold, as compared to a reference sequence of SEQ ID NO: 138, e.g., when measured by an assay, e.g., an IHC assay or a RT-ddPCR assay (e.g., as described in Example 3), optionally wherein the muscle region comprises a heart muscle (e.g., a heart atrium muscle region or a heart ventricle muscle region), quadriceps muscle, and/or a diaphragm muscle region.
14. The isolated AAV particle of any one of embodiments 1-13, wherein the AAV capsid variant comprises:
   (i) a VP1 protein, a VP2 protein, a VP3 protein, or a combination thereof;
   (ii) the amino acid sequence corresponding to positions 138-743, e.g., a VP2, of any one of SEQ ID NOs: 3636-3647, or a sequence with at least 90% (e.g., at least about 95, 96, 97, 98, or 99%) sequence identity thereto;
   (iii) the amino acid sequence corresponding to positions 203-743, e.g., a VP3, of any one of SEQ ID NOs: 3636-3647, or a sequence with at least 90% (e.g., at least about 95, 96, 97, 98, or 99%) sequence identity thereto;
   (iv) the amino acid sequence of any one of SEQ ID NOs: 3636-3647, or an amino acid sequence with at least 90% (e.g., at least about 95, 96, 97, 98, or 99%) sequence identity thereto;
   (v) an amino acid sequence having at least one, two or three modifications, but not more than 30, 20 or 10 modifications, e.g., substitutions (e.g., conservative substitutions), of the amino acid sequence of any one of SEQ ID NOs: 3636-3647;
   (vi) an amino acid sequence having at least one, two or three, but no more than 30, 20 or 10 different amino acids relative to the amino acid sequence of any one of SEQ ID NOs: 3636-3647; and/or
   (vii) an amino acid sequence encoded by the nucleotide sequence of any one of SEQ ID NOs: 3623-3635, or a nucleotide sequence with at least 90% (e.g., at least about 95, 96, 97, 98, or 99%) sequence identity thereto.
15. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises an amino sequence comprising the following formula: [N1]-[N2], wherein:
   (i) [N1] comprises X1, X2, X3, X4, and X5, wherein:
      (a) position X1 is: P, Q, A, H, K, L, R, S, or T;
      (b) position X2 is: L, I, V, H, or R;
      (c) position X3 is: N, D, I, K, or Y;
      (d) position X4: G, A, C, R, or S; and
      (e) position X5: A, S, T, G, C, D, N, Q, V, or Y; and
   (ii) [N2] comprises the amino acid sequence of VHLY (SEQ ID NO: 3741), VHIY (SEQ ID NO: 3746), VHVY (SEQ ID NO: 3742), or VHHY(SEQ ID NO: 3747); and/or an amino acid modification, e.g., a conservative substitution, of any of the aforesaid amino acids in (i) and/or (ii);
   optionally wherein the AAV capsid variant further comprises:
   (i) one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138; and/or
   (ii) one, two, or all of an amino acid other than V at position 596 (e.g., D, F, G, L, A, E, or I), an amino acid other than Q at position 597 (e.g., P, K, R, H, E, or L), and/or an amino acid other than N at position 598 (e.g., T, K, H, D, Y, S, I, or P), relative to a reference sequence numbered according to the amino acid sequence of SEQ ID NO: 138.
16. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises one, two, three, four, or all of:
   (i) an [N1], wherein [N1] is or comprises: SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), PLDSS (SEQ ID NO: 3723), PLNGC (SEQ ID NO: 3724), PLYGA (SEQ ID NO: 3725), TLNGA (SEQ ID NO: 3726), PVDGA (SEQ ID NO: 3727), PLKGA (SEQ ID NO: 3728), PLNGD (SEQ ID NO: 3729), KLDGA (SEQ ID NO: 3730), PHNGA (SEQ ID NO: 3731), PLNGV (SEQ ID NO: 3732), PLNAA (SEQ ID NO: 3733), QLNGY (SEQ ID NO: 3734), PLDGS (SEQ ID NO: 3735), LLNGA (SEQ ID NO: 3736), PLNRA (SEQ ID NO: 3737), PLIGA (SEQ ID NO: 3738), PRNGA (SEQ ID NO: 3739), or ALNGS (SEQ ID NO: 3740);
   (ii) an [N2] wherein [N2] is or comprises: VHLY (SEQ ID NO: 3741), VHVY (SEQ ID NO: 3742), VPLY (SEQ ID NO: 3743), VNLY (SEQ ID NO: 3744), VHRY (SEQ ID NO: 3745), VHIY (SEQ ID NO: 3746), VHHY (SEQ ID NO: 3747), FHLY (SEQ ID NO: 3748), LHLY (SEQ ID NO: 3749), DHLY (SEQ ID NO: 3750), VQLY (SEQ ID NO: 3751), IHLY(SEQ ID NO: 3752), VDLY(SEQ ID NO: 3753), AHLY (SEQ ID NO: 3754), VLLY (SEQ ID NO: 3755), GHLY (SEQ ID NO: 3756), VRLY, (SEQ ID NO: 3757) or VYLY (SEQ ID NO: 3758);
   (iii) an [N3] wherein [N3] is or comprises: AQAQ (SEQ ID NO: 3759), SQAQ (SEQ ID NO: 3760), AQPQ (SEQ ID NO: 3761), AQSQ (SEQ ID NO: 3762), AKAQ (SEQ ID NO: 3763), AHAQ (SEQ ID NO: 3764), AQAP (SEQ ID NO: 3765), DQAQ (SEQ ID NO: 3766), APAQ (SEQ ID NO: 3767), AQAK (SEQ ID NO: 3768), AQAH (SEQ ID NO: 3769), AQEQ (SEQ ID NO: 3770), ALAQ (SEQ ID NO: 3771), ARAQ SEQ ID NO: 3772), or TQAQ (SEQ ID NO: 3773);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW; and/or
   (v) an [N5] wherein [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH; and/or
   wherein the AAV capsid variant comprises an amino acid modification, e.g., a conservative substitution, of any of the aforesaid amino acids in (i)-(v).
17. The isolated AAV particle of embodiment 15, wherein:
   (a) position X1 is: P, Q, A, S, or T;
   (b) position X2 is L or I;
   (c) position X3 is N or D;
   (d) position X4 is G or S; and/or
   (e) position X5 is: A, S, G, T, or N.
18. The isolated AAV particle of embodiment 15 or 17, wherein [N1] comprises AL, PI, PL, QL, SL, TL, LN, LD, IN, NG, DG, DS, GA, SA, SS, GG, GN, GS, or GT.
19. The isolated AAV particle of any one of embodiments 15, 17, or 18, wherein [N1] comprises ALD, ALN, PIN, PLD, PLN, QLN, SLD, SLN, TLN, LNG, LDG, ING, LDS, NGA, DGA, DSA, DSS, NGG, NGN, NGS, NGT.
20. The isolated AAV particle of any one of embodiments 15 or 17-19, wherein [N1] comprises ALDG (SEQ ID NO: 3774), ALNG (SEQ ID NO: 3775), PING (SEQ ID NO: 3776), PLDG (SEQ ID NO: 3777), PLDS (SEQ ID NO: 3778), PLNG (SEQ ID NO: 3678), QLNG (SEQ ID NO: 3779), SLDG (SEQ ID NO: 3780), SLNG (SEQ ID NO: 3781), or TLNG (SEQ ID NO: 3782).
21. The isolated AAV particle of any one of embodiments 15-20, wherein [N1] is or comprises SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), PLDSS (SEQ ID NO: 3723), PLNGC (SEQ ID NO: 3724), PLYGA (SEQ ID NO: 3725), TLNGA (SEQ ID NO: 3726), PVDGA (SEQ ID NO: 3727), PLKGA (SEQ ID NO: 3728), PLNGD (SEQ ID NO: 3729), KLDGA (SEQ ID NO: 3730), PHNGA (SEQ ID NO: 3731), PLNGV (SEQ ID NO: 3732), PLNAA (SEQ ID NO: 3733), QLNGY (SEQ ID NO: 3734), PLDGS (SEQ ID NO: 3735), LLNGA (SEQ ID NO: 3736), PLNRA (SEQ ID NO: 3737), PLIGA (SEQ ID NO: 3738), PRNGA (SEQ ID NO: 3739), or ALNGS (SEQ ID NO: 3740).
22. The isolated AAV particle of embodiment 15-21, wherein [N1] is or comprises ALDGA (SEQ ID NO: 3716), ALNGA (SEQ ID NO: 3704), PINGA (SEQ ID NO: 3715), PLDGA (SEQ ID NO: 3709), PLDSA (SEQ ID NO: 3719), PLDSS (SEQ ID NO: 3723), PLNGA (SEQ ID NO: 3679), PLNGG (SEQ ID NO: 3707), PLNGN (SEQ ID NO: 3711), PLNGS (SEQ ID NO: 3705), PLNGT (SEQ ID NO: 3708), QLNGA (SEQ ID NO: 3703), SLDGA (SEQ ID NO: 3712), SLNGA (SEQ ID NO: 3702), or TLNGA (SEQ ID NO: 3726).
23. The isolated AAV particle of embodiment 15-22, wherein [N1] is or comprises PLNGA (SEQ ID NO: 3679).
24. The isolated AAV particle of any one of embodiments 15 or 17-18 or 20-23, wherein [N1]-[N2] comprises:

(i)
(SEQ ID NO: 3783)
LDGAVHLY,
(SEQ ID NO: 3784)
LNGAVHLY,
(SEQ ID NO: 3785)
INGAVHLY,
(SEQ ID NO: 3786)
LDSAVHLY,
(SEQ ID NO: 3787)
LDSSVHLY,
(SEQ ID NO: 3788)
LNGGVHLY,
(SEQ ID NO: 3789)
LNGNVHLY,
(SEQ ID NO: 3790)
LNGSVHLY,
(SEQ ID NO: 3791)
LNGTVHLY,
(SEQ ID NO: 3792)
LNGAVHIY,
(SEQ ID NO: 3793)
LDGAVHVY,
(SEQ ID NO: 3794)
LNGAVHHY;

(ii) an amino acid sequence comprising any portion of an amino acid sequence in (i), e.g., any 2, 3, 4, 5, 6, or 7 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
25. The isolated AAV particle of any one of embodiments 15-24, wherein [N1]-[N2] is or comprises:

(i)
(SEQ ID NO: 3795)
ALDGAVHLY,
(SEQ ID NO: 3704)
ALNGAVHLY,
(SEQ ID NO: 3796)
PINGAVHLY,
(SEQ ID NO: 3797)
PLDGAVHLY,
(SEQ ID NO: 3798)
PLDSAVHLY,
(SEQ ID NO: 3799)
PLDSSVHLY,
(SEQ ID NO: 3648)
PLNGAVHLY,
(SEQ ID NO: 3800)
PLNGGVHLY,
(SEQ ID NO: 3801)
PLNGNVHLY,
(SEQ ID NO: 3802)
PLNGSVHLY,
(SEQ ID NO: 3803)
PLNGTVHLY,
(SEQ ID NO: 3804)
QLNGAVHLY,
(SEQ ID NO: 3805)
SLDGAVHLY,
(SEQ ID NO: 3806)
SLNGAVHLY,
(SEQ ID NO: 3807)
TLNGAVHLY,
(SEQ ID NO: 3808)
PLNGAVHIY,
(SEQ ID NO: 3809)
PLDGAVHVY,
(SEQ ID NO: 3810)
PLNGAVHHY;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
26. The isolated AAV particle of any one of embodiments 15-25, wherein [N1]-[N2] is or comprises PLNGAVHLY (SEQ ID NO: 3648).
27. The isolated AAV particle of any one of embodiments 15-26, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P, E, or R), and/or an amino acid other than Q at position 592 (e.g., H, K, or P).
28. The isolated AAV particle of any one of embodiments 15-27, wherein the AAV capsid variant further comprises one, two, three, or all of an amino acid other than A at position 596 (e.g., D, S, or T), an amino acid other than Q at position 597 (e.g., K, H, L, P, or R), an amino acid other than A at position 598 (e.g., P, E, or R), and/or an amino acid other than Q at position 599 (e.g., H, K, or P), numbered according to the amino acid sequence of SEQ ID NO: 5, 8, or 3636.
29. The isolated AAV particle of any one of embodiments 15-26, wherein the AAV capsid variant further comprises
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
30. The isolated AAV particle of any one of embodiments 15 or 17-29, wherein the AAV capsid variant further comprises [N3], wherein [N3] comprises X6, X7, X8, and X9, wherein:
   (a) position X6 is: A, D, S, or T;
   (b) position X7 is: Q, K, H, L, P, or R;
   (c) position X8 is: A, P, E, or R; and
   (d) position X9 is: Q, H, K, or P; and/or
an amino acid modification, e.g., a conservative substitution, of any of the aforesaid amino acids in (a)-(d).
31. The isolated AAV particle of embodiment 30, wherein:
   (a) position X6 is: A, D, S;
   (b) position X7 is Q or K;
   (c) position X8 is A or P; and/or
   (d) position X9 is Q.
32. The isolated AAV particle of embodiment 30 or 31, wherein [N3] comprises AQ, SQ, AK, DQ, PQ, QA, QP, or KA.
33. The isolated AAV particle of any one of embodiments 30-32, wherein [N3] comprises AQA, AQP, SQA, AKA, DQA, QAQ, QPQ, or KAQ.
34. The isolated AAV particle of any one of embodiments 30-33, wherein [N3] is or comprises AQAQ (SEQ ID NO: 3759), SQAQ (SEQ ID NO: 3760), AQPQ (SEQ ID NO: 3761), AQSQ (SEQ ID NO: 3762), AKAQ (SEQ ID NO: 3763), AHAQ (SEQ ID NO: 3764), AQAP (SEQ ID NO: 3765), DQAQ (SEQ ID NO: 3766), APAQ (SEQ ID NO: 3767), AQAK (SEQ ID NO: 3768), AQAH (SEQ ID NO: 3769), AQEQ (SEQ ID NO: 3770), ALAQ (SEQ ID NO: 3771), ARAQ SEQ ID NO: 3772), or TQAQ (SEQ ID NO: 3773).
35. The isolated AAV particle of any one of embodiments 16 or 30-34, wherein [N3] is or comprises AQAQ (SEQ ID NO: 3759), AQPQ (SEQ ID NO: 3761), SQAQ (SEQ ID NO: 3760), AKAQ (SEQ ID NO: 3763), or DQAQ (SEQ ID NO: 3766).
36. The isolated AAV particle of any one of embodiments 16 or 30-35, wherein [N3] is or comprises AQAQ.
37. The isolated AAV article of an one of embodiments 16 or 30-36, wherein [N2]-[N3] comprises:

(i)
(SEQ ID NO: 3811)
VHLYAQAQ,
(SEQ ID NO: 3812)
VHLYAQPQ,
(SEQ ID NO: 3813)
VHLYSQAQ,
(SEQ ID NO: 3814)
VHLYAKAQ,
(SEQ ID NO: 3815)
VHLYDQAQ,
(SEQ ID NO: 3816)
VHIYAQAQ,
(SEQ ID NO: 3817)
VHVYAQAQ,
(SEQ ID NO: 3818)
VHHYAQAQ;

(ii) an amino acid sequence comprising any portion of an amino acid sequence in (i), e.g., any 2, 3, 4, 5, 6, or 7 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
38. The isolated AAV particle of any one of embodiments 30-37, wherein [N1]-[N2]-[N3] comprises:

(i)
(SEQ ID NO: 3819)
ALDGAVHLYAQ,
(SEQ ID NO: 3820)
ALNGAVHLYAQ,
(SEQ ID NO: 3821)
PINGAVHLYAQ,
(SEQ ID NO: 3822)
PLDGAVHLYAQ,
(SEQ ID NO: 3823)
PLDGAVHLYSQ,
(SEQ ID NO: 3824)
PLDSAVHLYAQ,
(SEQ ID NO: 3825)
PLDSSVHLYAQ,
(SEQ ID NO: 3826)
PLNGAVHLYAK,
(SEQ ID NO: 3827)
PLNGAVHLYAQ,
(SEQ ID NO: 3828)
PLNGAVHLYDQ,
(SEQ ID NO: 3829)
PLNGAVHLYSQ,
(SEQ ID NO: 3830)
PLNGGVHLYAQ,
(SEQ ID NO: 3831)
PLNGNVHLYAQ,
(SEQ ID NO: 3832)
PLNGSVHLYAQ,
(SEQ ID NO: 3833)
PLNGTVHLYAQ,
(SEQ ID NO: 3834)
QLNGAVHLYAQ,
(SEQ ID NO: 3835)
SLDGAVHLYAQ,
(SEQ ID NO: 3836)
SLNGAVHLYAQ,
(SEQ ID NO: 3837)
TLNGAVHLYAQ,
(SEQ ID NO: 3838)
PLNGAVHIYAQ,
(SEQ ID NO: 3839)
PLDGAVHVYAQ,
(SEQ ID NO: 3840)
PLNGAVHHYAQ;

(ii) an amino acid sequence comprising any portion of an amino acid sequence in (i), e.g., any 2, 3, 4, 5, 6, 7, 8, 9, or 10 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
39. The isolated AAV particle of any one of embodiments 16 or 30-38, wherein [N1]-[N2]-[N3] is or comprises:

(i)
(SEQ ID NO: 3841)
ALDGAVHLYAQAQ,
(SEQ ID NO: 3842)
ALNGAVHLYAQAQ,
(SEQ ID NO: 3843)
PINGAVHLYAQAQ,
(SEQ ID NO: 3844)
PLDGAVHLYAQAQ,
(SEQ ID NO: 3845)
PLDGAVHLYAQPQ,
(SEQ ID NO: 3846)
PLDGAVHLYSQAQ,
(SEQ ID NO: 3847)
PLDSAVHLYAQAQ,
(SEQ ID NO: 3848)
PLDSSVHLYAQAQ,
(SEQ ID NO: 3849)
PLNGAVHLYAKAQ,
(SEQ ID NO: 3850)
PLNGAVHLYAQAQ,
(SEQ ID NO: 3851)
PLNGAVHLYAQPQ,
(SEQ ID NO: 3852)
PLNGAVHLYDQAQ,
(SEQ ID NO: 3853)
PLNGAVHLYSQAQ,
(SEQ ID NO: 3854)
PLNGGVHLYAQAQ,
(SEQ ID NO: 3855)
PLNGNVHLYAQAQ,
(SEQ ID NO: 3856)
PLNGSVHLYAQAQ,
(SEQ ID NO: 3857)
PLNGTVHLYAQAQ,
(SEQ ID NO: 3858)
QLNGAVHLYAQAQ,
(SEQ ID NO: 3859)
SLDGAVHLYAQAQ,
(SEQ ID NO: 3860)
SLNGAVHLYAQAQ,
(SEQ ID NO: 3861)
TLNGAVHLYAQAQ,
(SEQ ID NO: 3862)
PLNGAVHIYAQAQ,
(SEQ ID NO: 3863)
PLDGAVHVYAQAQ,
(SEQ ID NO: 3864)
PLNGAVHHYAQAQ;

(ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
40. The isolated AAV particle of any one of embodiments 16 or 30-39, wherein [N1]-[N2]-[N3] is or comprises PLNGAVHLYAQAQ (SEQ ID NO: 3850).
41. The isolated AAV particle of any one of embodiments 15-40, wherein the AAV capsid variant further comprises one, two, or all of an amino acid other than T at position 593 (e.g., V, L, R, S, A, C, I, K, M, N, P, or Q), an amino acid other than G at position 594 (e.g., S, A, T, M, V, Q, L, H, I, K, N, P, R, or Y), and/or an amino acid other than W at position 595 (e.g., S, P, G, A, Q, L, M, K, C, E, F, H, R, T, V, or Y), relative to a reference sequence numbered according to the amino acid sequence of SEQ ID NO: 138.
42. The isolated AAV particle of any one of embodiments 15-41, wherein the AAV capsid variant further comprises one, two, or all of an amino acid other than T at position 600 (e.g., V, L, R, S, A, C, I, K, M, N, P, or Q), an amino acid other than G at position 601 (e.g., S, A, T, M, V, Q, L, H, I, K, N, P, R, or Y), and/or an amino acid other than W at position 602 (e.g., S, P, G, A, Q, L, M, K, C, E, F, H, R, T, V, or Y), relative to a reference sequence numbered according to the amino acid sequence of SEQ ID NO: 5, 8, or 3636.
43. The isolated AAV particle of any one of embodiments 15-42, wherein the AAV capsid variant further comprises one, two, or all of:
   (i) the amino acid V, L, R, S, A, C, I, K, M, N, P, or Q (e.g., L) at position 593 numbered according to SEQ ID NO: 138 or at position 600 numbered according to SEQ ID NO: 5, 8, or 3636);
   (ii) the amino acid S, A, T, M, V, Q, L, H, I, K, N, P, R, or Y (e.g., S) at position 594 numbered according to SEQ ID NO: 138, or at position 601 numbered according to SEQ ID NO: 5, 8, or 3636; and/or
   (iii) the amino acid S, P, G, A, Q, L, M, K, C, E, F, H, R, T, V, or Y (e.g. P) at position 595 numbered according to SEQ ID NO: 138 or at position 602 numbered according to SEQ ID NO: 5, 8, or 3636).
44. The isolated AAV particle of any one of embodiments 15-43, wherein the AAV capsid variant further comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636.
45. The isolated A AV particle of any one of embodiments 15-41, wherein the AAV capsid variant further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
46. The isolated AAV particle of any one of embodiments 16-45, wherein the AAV capsid variant further comprises [N4], wherein [N4] comprises X10, X11, and X12, wherein:
   (a) position X10 is: T, V, L, R, S, A, C, I, K, M, N, P, or Q;
   (b) position X11 is: G, S, A, T, M, V, Q, L, H, I, K, N, P, R, or Y; and
   (c) position X12 is: W, S, P, G, A, Q, L, M, K, C, E, F, H, R, T, V, or Y; and/or
   an amino acid modification, e.g., a conservative substitution, of any of the aforesaid amino acids in (a)-(c).
47. The isolated AAV particle of embodiment 46, wherein:
   (a) position X10 is: T, V, L, A, R, C, S, I, M, N, P, Q;
   (b) position X11 is: G, A, S, T, M, Q, V; and/or
   (c) position X12 is: P, S, W, G, A, Q, T, K, N, R, L, M, H, V, C, or E.
48. The isolated AAV particle of any one of embodiments 46 or 47, wherein [N4] comprises LS, TG, LA, LT, SA, SS, TL, TT, TS, TA, TV, VS, AA, AG, AS, AT, CS, CT, IA, IG, IL, IQ, IS, IT, LG, LH, LK, LM, LN, LQ, MA, NA, NM, NS, NT, NV, QA, RA, RG, RI, RL, RM, RN, RQ, RS, RT, RV, SG, SM, ST, SV, TK, TM, TN, TP, TQ, TR, VA, VG, VH, VK, VL, VM, VN, VQ, VR, VT, PG, LV, SP, GW, AP, GR, AL, AW, GG, GS, GP, QP, QS, AH, AN, AQ, AR, GQ, HP, KS, MG, MP, MQ, MS, NP, QQ, QR, SH, SK, SQ, SR, IP, VE, AK, AM, AV, GA, GC, GT, KA, KP, KQ, LP, MK, MN, MT, NQ, PP, QH, QK, QM, QN, QT, RW, SL, VW, GK, GN, NG, RP, SN, GL, or VP.
49. The isolated AAV particle of any one of embodiments 16 or 46-48, wherein [N4] is or comprises TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW.
50. The isolated AAV particle of any one of embodiments 16 or 46-49, wherein [N4] is or comprises LSP, TGW, LAA, LTP, SAP, SSP, TGR, TLA, TTS, TSP, TAL, TAW, TGG, TGS, TVS, VSP, VSS, AAP AGP, ASP, ATP, CSP, CTP, IAA, IAG, IAS, IGG, IGS, ILG, IQP, IQS, ISG, ISP, ISS, ITP, LAG, LAH, LAN, LAP, LAQ, LAR, LAS, LAT, LGP, LGQ, LGS, LHP, LKS, LMA, LMG, LMP, LMQ, LMS, LNP, LQP, LQQ, LQR, LSH, LSK, LSQ, LSR, LST, LTA, LTN, LTS, MAP, NAQ, NAS, NMQ, NSP, NTP, NVQ, QAP, RAA, RAQ, RAS, RGG, RGS, RIA, RIG, RIP, RLG, RLS, RMS, RNS, RQP, RSA, RSG, RSP, RSQ, RSS, RST, RTA, RTG, RTL, RTS, RTT, RVE, SAA, SAK, SAM, SAQ, SGP, SMA, SMG, SMQ, SMS, STP, SVA, SVG, TAA, TAG, TAK, TAM, TAN, TAP, TAQ, TAS, TAT, TAV, TGA, TGC, TGP, TGT, TKA, TKP, TKQ, TKS, TLP, TLQ, TMA, TMG, TMK, TMN, TMP, TMQ, TMS, TMT, TNA, TNQ, TNS, TPP, TQH, TQK, TQM, TQN, TQP, TQQ, TQT, TRW, TSA, TSG, TSH, TSK, TSL, TSM, TSQ, TSS, TST, TSV, TTA, TTG, TTK, TTP, TTQ, TTT, TVA, TVG, TVQ, TVW, VAA, VAG, VAK, VAN, VAQ, VAS, VAT, VGG, VGK, VGN, VGS, VHP, VKA, VKP, VKQ, VLP, VLS, VMA, VMQ, VMS, VNA, VNG, VNS, VQA, VQN, VQP, VQQ, VQS, VQT, VRP, VSA, VSG, VSN, VSQ, VSR, VST, VTA, VTG, VTK, VTP, VTS, TGL, PGW, LSG, LSS, or LVP.
51. The isolated AAV particle of any one of embodiments 15 or 46-50, wherein [N4] is or comprises TGW.
52. The isolated AAV particle of any one of embodiments 16 or 46-50, wherein [N4] is or comprises LSP.
53. The isolated AAV particle of any one of embodiments 15-52, wherein the AAV capsid variant further comprises one, two, or all of an amino acid other than V at position 596 (e.g., D, F, G, L, A, E, or I), an amino acid other than Q at position 597 (e.g., K, R, H, E, L, or P), and/or an amino acid other than N at position 598 (e.g., T, K, H, D, Y, S, I, or P), relative to a reference sequence numbered according to the amino acid sequence of SEQ ID NO: 138.
54. The isolated AAV particle of any one of embodiments 15-53, wherein the AAV capsid variant further comprises one, two, or all of an amino acid other than V at position 603 (e.g., D, F, G, L, A, E, or I), an amino acid other than Q at position 604 (e.g., K, R, H, E, L, or P), and/or an amino acid other than N at position 605 (e.g., T, K, H, D, Y, S, I, or P), relative to a reference sequence numbered according to the amino acid sequence of SEQ ID NO: 5, 8, or 3636.
55. The isolated AAV particle of any one of embodiments 15-54, wherein the AAV capsid variant further comprises one, two, or all of:
   (i) the amino acid V, D, F, G, L, A, E, or I at position 596 numbered according to SEQ ID NO: 138 or at position 603 numbered according to SEQ ID NO: 5, 8, or 3636;
   (ii) the amino acid K, R, H, E, L, or P at position 597 numbered according to SEQ ID NO: 138 or at position 604 numbered according to SEQ ID NO: 5, 8, or 3636; and/or
   (iii) the amino acid N, T, K, H, D, Y, S, I, or P at position 598 numbered according to the amino acid sequence of SEQ ID NO: 138 or at position 605 numbered according to SEQ ID NO: 5, 8, or 3636.
56. The isolated AAV particle of any one of embodiments 15-55, wherein the AAV capsid variant further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
57. The isolated AAV particle of any one of embodiments 16-56, wherein the AAV capsid variant further comprises [N5], wherein [N5] comprises X13, X14, and X15, wherein:
   (a) position X13 is: V, D, F, G, L, A, E, or I;
   (b) position X14 is: Q, K, R, H, E, L, or P; and
   (c) position X15 is: N, T, K, H, D, Y, S, I, or P; and/or
an amino acid modification, e.g., a conservative substitution, of any of the aforesaid amino acids in (a)-(c).
58. The isolated AAV particle of embodiment 57, wherein:
   (a) position X13 is: V, D, A, F, E, G, or L;
   (b) position X14 is: Q, K, R, L, or P; and/or
   (c) position X15 is: N, T, K, H, D, I, K, S, or P.
59. The isolated AAV particle of embodiment 57 or 58, wherein position X14 is P.
60. The isolated AAV particle of embodiment 57 or 58, wherein position X14 is K.
61. The isolated AAV particle of embodiment 57 or 58, wherein position X14 is E or H.
62. The isolated AAV particle of embodiment 57 or 58, wherein position X14 is Q.
63. The isolated AAV particle of embodiment 57 or 62, wherein [N5] comprises VQ, AQ, DQ, FQ, VL, LQ, EQ, GQ, VP, VR, VK, QN, QS, QT, QK, QH, LN, QI, PN, QD, QP, RN, or KN.
64. The isolated AAV particle of any one of embodiments 16 or 57-63, wherein [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH.
65. The isolated AAV particle of any one of embodiments 16 or 57-64, wherein [N5] is or comprises VQN, AQN, VQS, DQN, VQT, VQK, VQH, FQN, VLN, LQN, VQI, EQN, GQT, VPN, VQD, VQP, VRN, or VKN.
66. The isolated AAV particle of any one of embodiments 16 or 57-65, wherein [N5] is or comprises VKN, VPN, VEN, or VHN.
67. The isolated AAV particle of any one of embodiments 16 or 57-64, wherein [N5] is or comprises VQN.
68. The isolated AAV particle of any one of embodiments 16 or 47-67, wherein [N4]-[N5] is or comprises:

(i)
(SEQ ID NO: 3865)
TGWVQN,
(SEQ ID NO: 3866)
LAAVQN,
(SEQ ID NO: 3867)
LTPVQN,
(SEQ ID NO: 3868)
SAPVQN,
(SEQ ID NO: 3869)
SSPVQN,
(SEQ ID NO: 3870)
TGRVQN,
(SEQ ID NO: 3871)
TGWAQN,
(SEQ ID NO: 3872)
TGWVQS,
(SEQ ID NO: 3873)
TLAVQN,
(SEQ ID NO: 3874)
TTSVQN,
(SEQ ID NO: 3875)
TSPVQN,
(SEQ ID NO: 3876)
TALVQN,
(SEQ ID NO: 3877)
TAWVQN,
(SEQ ID NO: 3878)
TGGVQN,
(SEQ ID NO: 3879)
TGSVQN,
(SEQ ID NO: 3880)
TGWDQN,
(SEQ ID NO: 3881)
TVSVQN,
(SEQ ID NO: 3882)
VSPVQN,
(SEQ ID NO: 3883)
VSSVQN,
(SEQ ID NO: 3884)
AAPVQN,
(SEQ ID NO: 3885)
AGPVQN,
(SEQ ID NO: 3886)
ASPVQN,
(SEQ ID NO: 3887)
ATPVQN,
(SEQ ID NO: 3888)
CSPVQN,
(SEQ ID NO: 3889)
CTPVQN,
(SEQ ID NO: 3890)
IAAVQN,
(SEQ ID NO: 3891)
IAGVQN,
(SEQ ID NO: 3892)
IASVQN,
(SEQ ID NO: 3893)
IGGVQN,
(SEQ ID NO: 3894)
IGSVQN,
(SEQ ID NO: 3895)
ILGVQN,
(SEQ ID NO: 3896)
IQPVQN,
(SEQ ID NO: 3897)
IQSVQN,
(SEQ ID NO: 3898)
ISGVQN,
(SEQ ID NO: 3899)
ISPVQN,
(SEQ ID NO: 3900)
ISSVQN,
(SEQ ID NO: 3901)
ITPVQN,
(SEQ ID NO: 3902)
LAGVQN,
(SEQ ID NO: 3903)
LAHVQN,
(SEQ ID NO: 3904)
LANVQN,
(SEQ ID NO: 3905)
LAPVQN,
(SEQ ID NO: 3906)
LAPVQT,
(SEQ ID NO: 3907)
LAQVQN,
(SEQ ID NO: 3908)
LARVQN,
(SEQ ID NO: 3909)
LASVQN,
(SEQ ID NO: 3910)
LATVQN,
(SEQ ID NO: 3911)
LGPVQN,
(SEQ ID NO: 3912)
LGQVQN,
(SEQ ID NO: 3913)
LGSVQN,
(SEQ ID NO: 3914)
LHPVQN,
(SEQ ID NO: 3915)
LKSVQN,
(SEQ ID NO: 3916)
LMAVQN,
(SEQ ID NO: 3920)
LMGVQN,
(SEQ ID NO: 3921)
LMPVQN,
(SEQ ID NO: 3922)
LMQVQN,
(SEQ ID NO: 3923)
LMSVQN,
(SEQ ID NO: 3924)
LNPVQN,
(SEQ ID NO: 3925)
LQPVQN,
(SEQ ID NO: 3926)
LQQVQN,
(SEQ ID NO: 3927)
LQRVQN,
(SEQ ID NO: 3928)
LSHVQN,
(SEQ ID NO: 3929)
LSKVQN,
(SEQ ID NO: 3930)
LSPVQK,
(SEQ ID NO: 3931)
LSPVQN,
(SEQ ID NO: 3932)
LSQVQN,
(SEQ ID NO: 3933)
LSRVQN,
(SEQ ID NO: 3934)
LSTVQN,
(SEQ ID NO: 3935)
LTAVQN,
(SEQ ID NO: 3936)
LTNVQN,
(SEQ ID NO: 3937)
LTSVQN,
(SEQ ID NO: 3938)
MAPVQN,
(SEQ ID NO: 3939)
NAQVQN,
(SEQ ID NO: 3940)
NASVQN,
(SEQ ID NO: 3941)
NMQVQN,
(SEQ ID NO: 3942)
NSPVQN,
(SEQ ID NO: 3943)
NTPVQN,
(SEQ ID NO: 3944)
NVQVQN,
(SEQ ID NO: 3945)
QAPVQN,
(SEQ ID NO: 3946)
RAAVQN,
(SEQ ID NO: 3947)
RAQVQN,
(SEQ ID NO: 3948)
RASVQN,
(SEQ ID NO: 3949)
RGGVQN,
(SEQ ID NO: 3950)
RGSVQN,
(SEQ ID NO: 3951)
RIAVQN,
(SEQ ID NO: 3952)
RIGVQN,
(SEQ ID NO: 3953)
RIPVQN,
(SEQ ID NO: 3954)
RLGVQN,
(SEQ ID NO: 3955)
RLSVQN,
(SEQ ID NO: 3956)
RMSVQN,
(SEQ ID NO: 3957)
RNSVQN,
(SEQ ID NO: 3958)
RQPVQN,
(SEQ ID NO: 3959)
RSAVQN,
(SEQ ID NO: 3960)
RSGVQN,
(SEQ ID NO: 3961)
RSPVQN,
(SEQ ID NO: 3962)
RSQVQN,
(SEQ ID NO: 3963)
RSSVQN,
(SEQ ID NO: 3964)
RSTVQN,
(SEQ ID NO: 3965)
RTAVQN,
(SEQ ID NO: 3966)
RTGVQN,
(SEQ ID NO: 3967)
RTLVQN,
(SEQ ID NO: 3968)
RTSVQN,
(SEQ ID NO: 3969)
RTTVQN,
(SEQ ID NO: 3970)
RVEVQN,
(SEQ ID NO: 3971)
SAAVQN,
(SEQ ID NO: 3972)
SAKVQN,
(SEQ ID NO: 3973)
SAMVQN,
(SEQ ID NO: 3974)
SAQVQN,
(SEQ ID NO: 3975)
SGPVQN,
(SEQ ID NO: 3976)
SMAVQN,
(SEQ ID NO: 3977)
SMGVQN,
(SEQ ID NO: 3978)
SMQVQN,
(SEQ ID NO: 3979)
SMSVQN,
(SEQ ID NO: 3980)
STPVQN,
(SEQ ID NO: 3981)
SVAVQN,
(SEQ ID NO: 3982)
SVGVQN,
(SEQ ID NO: 3983)
TAAVQN,
(SEQ ID NO: 3984)
TAGVQN,
(SEQ ID NO: 3985)
TAKVQN,
(SEQ ID NO: 3986)
TAMVQN,
(SEQ ID NO: 3987)
TANVQN,
(SEQ ID NO: 3988)
TAPVQN,
(SEQ ID NO: 3989)
TAPVQT,
(SEQ ID NO: 3990)
TAQVQN,
(SEQ ID NO: 3991)
TASVON,
(SEQ ID NO: 3992)
TASVQT,
(SEQ ID NO: 3993)
TATVQN,
(SEQ ID NO: 3994)
TAVVON,
(SEQ ID NO: 3995)
TAWDON,
(SEQ ID NO: 3996)
TAWVQH,
(SEQ ID NO: 3997)
TAWVQT,
(SEQ ID NO: 3998)
TGAVQN,
(SEQ ID NO: 3999)
TGCFQN,
(SEQ ID NO: 3672)
TGGAQN,
(SEQ ID NO: 3673)
TGGFQN,
(SEQ ID NO: 3674)
TGGVLN,
(SEQ ID NO: 3675)
TGGVQH,
(SEQ ID NO: 3676)
TGGVQK,
(SEQ ID NO: 3677)
TGGVQT,
(SEQ ID NO: 3696)
TGPVQN,
(SEQ ID NO: 3697)
TGSAQN,
(SEQ ID NO: 3697)
TGSLQN,
(SEQ ID NO: 3698)
TGSVQH,
(SEQ ID NO: 3699)
TGSVQI,
(SEQ ID NO: 1139)
TGSVQS,
(SEQ ID NO: 1140)
TGSVQT,
(SEQ ID NO: 1141)
TGTVQN,
(SEQ ID NO: 1142)
TGWEQN,
(SEQ ID NO: 1143)
TGWFQN,
(SEQ ID NO: 1144)
TGWGQT,
(SEQ ID NO: 1145)
TGWVPN,
(SEQ ID NO: 1146)
TGWVQD,
(SEQ ID NO: 1147)
TGWVQP,
(SEQ ID NO: 1148)
TGWVQT,
(SEQ ID NO: 1149)
TGWVRN,
(SEQ ID NO: 1150)
TKAVQN,
(SEQ ID NO: 1151)
TKPVQN,
(SEQ ID NO: 1152)
TKQVQN,
(SEQ ID NO: 1153)
TKSVQN,
(SEQ ID NO: 1154)
TLPVQN,
(SEQ ID NO: 1155)
TLQVQN,
(SEQ ID NO: 1156)
TMAVQN,
(SEQ ID NO: 1157)
TMGVQN,
(SEQ ID NO: 1158)
TMKVQN,
(SEQ ID NO: 1159)
TMNVQN,
(SEQ ID NO: 1160)
TMPVQN,
(SEQ ID NO: 1161)
TMQVQN,
(SEQ ID NO: 1162)
TMSVKN,
(SEQ ID NO: 1163)
TMSVQN,
(SEQ ID NO: 1164)
TMSVQT,
(SEQ ID NO: 1165)
TMTVQN,
(SEQ ID NO: 1166)
TNAVQN,
(SEQ ID NO: 1167)
TNQVQN,
(SEQ ID NO: 1168)
TNSVQN,
(SEQ ID NO: 1169)
TPPVQN,
(SEQ ID NO: 1170)
TQHVQN,
(SEQ ID NO: 1171)
TQKVQN,
(SEQ ID NO: 1172)
TQMVQN,
(SEQ ID NO: 1173)
TQNVQN,
(SEQ ID NO: 1174)
TQPVQN,
(SEQ ID NO: 1175)
TQQVQN,
(SEQ ID NO: 1176)
TQTVQN,
(SEQ ID NO: 1177)
TRWDQN,
(SEQ ID NO: 1178)
TSAVQN,
(SEQ ID NO: 1179)
TSGVQN,
(SEQ ID NO: 1180)
TSHVQN,
(SEQ ID NO: 1181)
TSKVQN,
(SEQ ID NO: 1182)
TSLVQN,
(SEQ ID NO: 1183)
TSMVQN,
(SEQ ID NO: 1184)
TSPDQN,
(SEQ ID NO: 1185)
TSQVQN,
(SEQ ID NO: 1186)
TSSVQN,
(SEQ ID NO: 1187)
TSSVQT,
(SEQ ID NO: 1188)
TSTVQN,
(SEQ ID NO: 1189)
TSVVQN,
(SEQ ID NO: 1190)
TTAVQN,
(SEQ ID NO: 1191)
TTGVQN,
(SEQ ID NO: 1192)
TTKVQN,
(SEQ ID NO: 1193)
TTPVQN,
(SEQ ID NO: 1194)
TTPVQT,
(SEQ ID NO: 1195)
TTQVQN,
(SEQ ID NO: 1196)
TTTVQN,
(SEQ ID NO: 1197)
TVAVQN,
(SEQ ID NO: 1198)
TVAVQT,
(SEQ ID NO: 1199)
TVGVQN,
(SEQ ID NO: 1200)
TVQVQN,
(SEQ ID NO: 1201)
TVSVKN,
(SEQ ID NO: 1202)
TVWVQK,
(SEQ ID NO: 1203)
VAAVQN,
(SEQ ID NO: 1204)
VAGVQN,
(SEQ ID NO: 1205)
VAKVON,
(SEQ ID NO: 1206)
VANVQN,
(SEQ ID NO: 1207)
VAQVQN,
(SEQ ID NO: 1208)
VASVQN,
(SEQ ID NO: 1209)
VATVQN,
(SEQ ID NO: 1210)
VGGVQN,
(SEQ ID NO: 1211)
VGKVQN,
(SEQ ID NO: 1212)
VGNVQN,
(SEQ ID NO: 1213)
VGSVQN,
(SEQ ID NO: 1214)
VHPVQN,
(SEQ ID NO: 1215)
VKAVQN,
(SEQ ID NO: 1216)
VKPVQN,
(SEQ ID NO: 1217)
VKQVQN,
(SEQ ID NO: 1218)
VLPVQN,
(SEQ ID NO: 1219)
VLSVQN,
(SEQ ID NO: 1220)
VMAVQN,
(SEQ ID NO: 1221)
VMQVQN,
(SEQ ID NO: 1222)
VMSVQN,
(SEQ ID NO: 1223)
VNAVQN,
(SEQ ID NO: 1224)
VNGVQN,
(SEQ ID NO: 1225)
VNSVQN,
(SEQ ID NO: 1226)
VQAVQN,
(SEQ ID NO: 1227)
VQNVON,
(SEQ ID NO: 1228)
VQPVQN,
(SEQ ID NO: 1229)
VQQVON,
(SEQ ID NO: 1230)
VQSVQN,
(SEQ ID NO: 1231)
VQTVQN,
(SEQ ID NO: 1232)
VRPVQN,
(SEQ ID NO: 1233)
VSAVQN,
(SEQ ID NO: 1234)
VSGVQN,
(SEQ ID NO: 1235)
VSNVQN,
(SEQ ID NO: 1236)
VSPVQT,
(SEQ ID NO: 1237)
VSQVQN,
(SEQ ID NO: 1238)
VSRVQN,
(SEQ ID NO: 1239)
VSSVQK,
(SEQ ID NO: 1240)
VSSVQT,
(SEQ ID NO: 1241)
VSTVQN,
(SEQ ID NO: 1242)
VTAVQN,
(SEQ ID NO: 1243)
VTGVQN,
(SEQ ID NO: 1244)
VTKVQN,
(SEQ ID NO: 1245)
VTPVQN,
(SEQ ID NO: 1246)
VTSVQN,
(SEQ ID NO: 1247)
TGLVQN,
(SEQ ID NO: 1248)
TGWVKN,
(SEQ ID NO: 1249)
PGWVQN,
(SEQ ID NO: 1250)
TGWVQH,
(SEQ ID NO: 1251)
LSGVQN,
(SEQ ID NO: 1252)
LSSVQN,
(SEQ ID NO: 1253)
LVPVQN;

(ii) an amino acid sequence comprising any portion of an amino acid sequence in (i), e.g., any 2, 3, 4, or 5 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).
69. The isolated AAV particle of any one of embodiments 16 or 57-68, wherein [N4]-[N5] is or comprises TGWVQN (SEQ ID NO: 3865), LSPVKN (SEQ ID NO: 1404), or TGWVPN (SEQ ID NO: 1145).
70. The isolated AAV particle of any one of embodiments 16-69, wherein:
   (i) [N1] is or comprises: QLNGA (SEQ ID NO: 3703), PLDGA (SEQ ID NO: 3709), PLDSS (SEQ ID NO: 3723), PLNGA (SEQ ID NO: 3679), ALNGA (SEQ ID NO: 3704), PLNGS (SEQ ID NO: 3705), PLNGG (SEQ ID NO: 3707), SLNGA (SEQ ID NO: 3702), PLNGN (SEQ ID NO: 3711), PLNGT (SEQ ID NO: 3708), ALDGA (SEQ ID NO: 3716), PLDSA (SEQ ID NO: 3719), SLDGA (SEQ ID NO: 3712), TLNGA (SEQ ID NO: 3726), or PINGA (SEQ ID NO: 3715);
   (ii) [N2] is or comprises: VHLY (SEQ ID NO: 3741), VHHY (SEQ ID NO: 3747), VHVY (SEQ ID NO: 3742), or VHIY(SEQ ID NO: 3746);
   (iii) [N3] is or comprises AQAQ (SEQ ID NO: 3759), AQPQ (SEQ ID NO: 3761), AKAQ (SEQ ID NO: 3763), DQAQ (SEQ ID NO: 3766), or SQAQ (SEQ ID NO: 3760);
   (iv) [N4] is or comprises: LSP, TGW, TMS, TTK, TGS, TTS, TSP, TMK, VAQ, TGG, TAW, VKQ, SAP, LSK, LAP, LAQ, VAS, TAK, SAK, TGC, TQK, TGR, TVA, SSP, TTQ, TAQ, RIA, RAS, TTP, LAS, LTP, STP, VSQ, TMQ, TSK, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, AGP, LAR, TTT, TLQ, VAK, RAA, TVG, LNP, LSQ, TKP, TNA, LAT, VTP, VQA, CTP, TAG, TSQ, TMN, TST, VKP, ASP, VAA, LKS, IAA, TAA, TKA, VSN, TAP, LMP, LHP, RAQ, LTN, RTT, TSV, TLA, RMS, VGN, LMQ, TAT, VHP, ISS, TRW, TMT, RSS, PGW, RTG, VAT, VTS, VSS, TSS, TNS, VKA, SGP, TGP, TAM, TQP, TQQ, VSR, VLP, LGS, VSA, VLS, TQH, QAP, NAQ, ATP, VQP, TTA, LAA, RSG, LMA, TMP, LAN, VST, SAQ, NTP, TGL, TLP, TAV, RLG, RTL, TQM, ITP, TVW, RSA, TAS, TMG, VQS, ISP, VGG, TAL, LAG, RTA, RSP, LAH, TSL, RLS, LMG, SMQ, TQT, VGS, VSG, VMA, IGG, IAG, LSH, VQT, RNS, TKQ, LGQ, NMQ, NVQ, RGG, VMS, TTG, LSR, MAP, ILG, TGT, TSH, RIG, SAM, TSM, SMG, SMS, TSG, TGA, VNS, VAG, IGS, VNG, LSS, LTA, VQN, TKS, SVG, NAS, TSA, TAN, LTS, RSQ, RIP, LVP, RVE, SVA, LSG, LQQ, LST, SAA, RTS, TQN, VNA, or LMS; and/or
   (v) [N5] is or comprises: VQN, DQN, VQH, FQN, VQD, VQS, VQT, VRN, AQN, VQP, VPN, VKN, VQK, EQN, VQI, LQN, GQT, or VLN.
71. The isolated AAV particle of embodiment 16, wherein:
   (i) [N1] is or comprises: SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), or PLDSS (SEQ ID NO: 3723);
   (ii) [N2] is or comprises: VHLY (SEQ ID NO: 3741) or VHVY (SEQ ID NO: 3742);
   (iii) [N3] is or comprises: AQAQ (SEQ ID NO: 3759), SQAQ (SEQ ID NO: 3760), AQPQ (SEQ ID NO: 3761), or AQSQ (SEQ ID NO: 3762);
   (iv) [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) [N5] is or comprises: VQN, VKN, VQT, VQK, DQN, VQH, GQN, VQI, VHN, FQN, LQN, VLN, VRN, VQS, VQY, AQN, VEN, VQD.
72. The isolated AAV particle of any one of embodiments 15, 16, 29, 30, 41, 46, 53 or 57, which comprises:
   (i) the amino acid sequence of any of SEQ ID NOs: 139-1138;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
73. The isolated AAV particle of any one of embodiments 15-70 or 72, which comprises:
   (i) the amino acid sequence of any of SEQ ID NOs: 139-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
74. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises one, two, three, four, or all of:
   (i) an [N1], wherein [N1] is or comprises: SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGA (SEQ ID NO: 3679), PLNGG (SEQ ID NO: (SEQ ID NO: 3707)), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), or PLDSS (SEQ ID NO: 3723);
   (ii) an [N2] wherein [N2] is or comprises: VHLY (SEQ ID NO: 3741) or VHVY (SEQ ID NO: 3742);
   (iii) an [N3] wherein [N3] is or comprises: AQAQ (SEQ ID NO: 3759), SQAQ (SEQ ID NO: 3760), AQPQ (SEQ ID NO: 3761), or AQSQ (SEQ ID NO: 3762);
   (iv) an [N4] wherein [N4] is or comprises: TGW, TGL, TGS, TGG, TAW, TGR, TAS, LSS, TSS, SSL, SSS, TLS, TVS, VSS, TSP, VSP, TMS, LSP, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, or TGC; and/or
   (v) an [N5] wherein [N5] is or comprises VQN, VKN, VQT, VQK, DQN, VQH, GQN, VQI, VHN, FQN, LQN, VLN, VRN, VQS, VQY, AQN, VEN, VQD; and/or
   wherein the AAV capsid variant comprises an amino acid modification, e.g., a conservative substitution, of any of the aforesaid amino acids in (i)-(v).
75. The isolated AAV particle of embodiment 71 or 74, wherein the AAV capsid variant comprises:
   (i) the amino acid sequence of any of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
76. The isolated AAV particle of any one of embodiments 16 or 57-75, wherein [N1]-[N2]-[N3]-[N4]-[N5] is or comprises the amino acid sequence of PLNGAVHLYAQAQTGWVPN (SEQ ID NO: 314).
77. The isolated AAV particle of any one of embodiments 16 or 57-75, wherein [N1]-[N2]-[N3]-[N4]-[N5] is or comprises the amino acid sequence of PLNGAVHLYAQAQLSPVKN (SEQ ID NO: 566).
78. The isolated AAV particle of any one of embodiments 16 or 57-65, wherein [N1]-[N2]-[N3]-[N4]-[N5] is or comprises the amino acid sequence of PLNGAVHLYAQAQTGWVQN (SEQ ID NO: 476).
79. The isolated AAV particle of any one of embodiments 16 or 57-74, wherein [N1]-[N2]-[N3]-[N4]-[N5] is or comprises the amino acid sequence of:
   (i) the amino acid sequence of any of SEQ ID NOs: 14-17, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1174-1193;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
80. The isolated AAV particle of any one of embodiments 15-79, wherein [N1]-[N2] is present in loop VIII.
81. The isolated AAV particle of any one of embodiments 16 or 29-80, wherein [N3], [N4], and/or [N5] is present in loop VIII.
82. The isolated AAV particle of any one of embodiments 15-81, wherein [N1]-[N2]-[N3]-[N4]-[N5] is present in loop VIII.
83. The isolated AAV particle of any one of embodiments 16-82, wherein the AAV capsid variant comprises an amino acid other than A at position 587 and/or an amino acid other than Q at position 588, numbered according to SEQ ID NO: 138.
84. The isolated AAV particle of any one of embodiments 15-83, which comprises:
   (i) the amino acid P, Q, A, H, K, L, R, S, or T (e.g., P, Q, A, S, or T) at position 587, numbered according to SEQ ID NO: 138 or 3636; and/or
   (ii) the amino acid L, I, V, H, or R (e.g., L or I) at position 588, numbered according to SEQ ID NO: 5, 8, 138 or 3636.
85. The isolated AAV particle of any one of embodiments 15-84, wherein [N1] is present immediately subsequent to position 586, relative to a reference sequence numbered according to the amino acid sequence of SEQ ID NO: 138, or 3636.
86. The isolated AAV particle of any one of embodiments 15-85, wherein [N1] is present immediately subsequent to position 586, and replaces positions 587 and 588 (e.g., A587 and Q588), numbered according to the amino acid sequence of SEQ ID NO: 138.
87. The isolated AAV particle of any one of embodiments 15-86, wherein [N1] replaces positions 587 and 588 (e.g., A587 and Q588), numbered according to the amino acid sequence of SEQ ID NO: 138.
88. The isolated AAV particle of any one of embodiments 15-86, wherein [N1] corresponds to positions 587-591 of SEQ ID NO: 5, 8, or 3636.
89. The isolated AAV particle of any one of embodiments 16 or 57-88, wherein [N1]-[N2]-[N3]-[N4]-[N5] is present immediately subsequent to position 586, numbered according to the amino acid sequence of SEQ ID NO: 5, 8, 138, or 3636.
90. The isolated AAV particle of any one of embodiments 15-89, wherein [N2] is present immediately subsequent to [N1].
91. The isolated AAV particle of any one of embodiments 15-90, wherein [N2] is present immediately subsequent to [N1], wherein [N1] is present immediately subsequent to position 586, numbered according to the amino acid sequence of SEQ ID NO: 5, 8, 138 or 3636.
92. The isolated AAV particle of any one of embodiments 15-91, wherein [N2] is present immediately subsequent to [N1], wherein [N1] is present immediately subsequent to position 586, relative to a reference sequence numbered according to the amino acid sequence of SEQ ID NO: 5, 8, 138, or 3636.
93. The isolated AAV particle of any one of embodiments 15-92, wherein [N2] is present immediately subsequent to [N1], wherein [N1] is present immediately subsequent to position 586 and replaces positions 587 and 588 (e.g., A587 and Q588), numbered according to SEQ ID NO: 138.
94. The isolated AAV particle of any one of embodiments 15-93, wherein [N2] corresponds to positions 592-595 of SEQ ID NO: 5, 8, or 3636.
95. The isolated AAV particle of any one of embodiments 15-94, wherein [N1]-[N2] replaces positions 587 and 588 (e.g., A587 and Q588), numbered according to SEQ ID NO: 138.
96. The isolated AAV particle of any one of embodiments 15-95, wherein [N1]-[N2] is present immediately subsequent to position 586, numbered according to SEQ ID NO: 138.
97. The isolated AAV particle of any one of embodiments 15-696, wherein [N1]-[N2] is present immediately subsequent to position 586 and replaces positions 587 and 588 (e.g., A587 and Q588), numbered according to SEQ ID NO: 138.
98. The isolated AAV particle of any one of embodiments 15-97, wherein [N1]-[N2] corresponds to positions 587-595 of SEQ ID NO: 5, 8, or 3636.
99. The isolated AAV particle of any one of embodiments 16 or 30-98, wherein [N3] is present immediately subsequent to position 588, relative to a reference sequence numbered according to the amino acid sequence of SEQ ID NO: 138.
100. The isolated AAV particle of any one of embodiments 15 or 30-99, wherein [N3] replaces positions 589-592 (e.g., A589, Q590, A591, Q592), numbered according to the amino acid sequence of SEQ ID NO: 138.
101. The isolated AAV particle of any one of embodiments 15 or 30-100, wherein [N3] is present immediately subsequent to position 588, and replaces positions 589-592 (e.g., A589, Q590, A591, Q592), numbered according to the amino acid sequence of SEQ ID NO: 138.
102. The isolated AAV particle of any one of embodiments 15 or 30-101, wherein [N3] corresponds to positions 596-599 of SEQ ID NO: 5, 8, or 3636.
103. The isolated AAV particle of any one of embodiments 15 or 30-102, wherein [N1]-[N2]-[N3] is present immediately subsequent to position 586, numbered according to SEQ ID NO: 138.
104. The isolated AAV particle of any one of embodiments 15 or 30-103, wherein [N1]-[N2]-[N3] replaces positions 587-592 (e.g., A587, Q588, A589, Q590, A591, Q592), numbered according to SEQ ID NO: 138.
105. The isolated AAV particle of any one of embodiments 15 or 30-104, wherein [N1]-[N2]-[N3] is present immediately subsequent to position 586 and replaces positions 587-592 (e.g., A587, Q588, A589, Q590, A591, Q592), numbered according to SEQ ID NO: 138.
106. The isolated AAV particle of any one of embodiments 15 or 30-105, wherein [N1]-[N2]-[N3] corresponds to positions 587-599 of SEQ ID NO: 5, 8, or 3636.
107. The isolated AAV particle of any one of embodiments 16 or 46-106, wherein [N4] is present immediately subsequent to position 592, relative to a reference sequence numbered according to the amino acid sequence of SEQ ID NO: 138.
108. The isolated AAV particle of any one of embodiments 16 or 46-102, wherein [N4] replaces positions 593-595 (e.g., T593, G594, W595), numbered according to the amino acid sequence of SEQ ID NO: 138.
109. The isolated AAV particle of any one of embodiments 16 or 46-108, wherein [N4] is present immediately subsequent to position 592, and replaces positions 593-595 (e.g., T593, G594, W595), numbered according to the amino acid sequence of SEQ ID NO: 138.
110. The isolated AAV particle of any one of embodiments 16 or 46-109, wherein [N4] corresponds to positions 600-602 of SEQ ID NO: 5, 8, or 3636.
111. The isolated AAV particle of any one of embodiments 16 or 46-110, wherein [N3]-[N4] is present immediately subsequent to position 588, numbered according to SEQ ID NO: 138.
112. The isolated AAV particle of any one of embodiments 16 or 46-111, wherein [N3]-[N4] replaces positions 589-595 (e.g., A589, Q590, A591, Q592, T593, G594, W595), numbered according to the amino acid sequence of SEQ ID NO: 138.
113. The isolated AAV particle of any one of embodiments 16 or 46-112, wherein [N3]-[N4] is present immediately subsequent to 588, and replaces positions 589-595 (e.g., A589, Q590, A591, Q592, T593, G594, W595), numbered according to the amino acid sequence of SEQ ID NO: 138.
114. The isolated AAV particle of any one of embodiments 16 or 46-113, wherein [N3]-[N4] corresponds to positions 596-602 of SEQ ID NO: 5, 8, or 3636.
115. The isolated AAV particle of any one of embodiments 16 or 46-114, wherein [N1]-[N2]-[N3]-[N4] is present immediately subsequent to position 586, numbered according to SEQ ID NO: 138.
116. The isolated AAV particle of any one of embodiments 16 or 46-115, wherein [N1]-[N2]-[N3]-[N4] replaces positions 587-595 (e.g., A587, Q588, A589, Q590, A591, Q592, T593, G594, W595), numbered according to SEQ ID NO: 138.
117. The isolated AAV particle of any one of embodiments 16 or 46-116, wherein [N1]-[N2]-[N3]-[N4] is present immediately subsequent to position 586 and replaces positions 587-595 (e.g., A587, Q588, A589, Q590, A591, Q592, T593, G594, W595), numbered according to SEQ ID NO: 138.
118. The isolated AAV particle of any one of embodiments 16 or 46-117, wherein [N1]-[N2]-[N3]-[N4] corresponds to positions 587-602 of SEQ ID NO: 5, 8, or 3636.
119. The isolated AAV particle of any one of embodiments 16 or 57-118, wherein [N5] is present immediately subsequent to position 595, relative to a reference sequence numbered according to the amino acid sequence of SEQ ID NO: 138.
120. The isolated AAV particle of any one of embodiments 16 or 57-119, wherein [N5] replaces positions 596-598 (e.g., V596, Q597, N598), numbered according to the amino acid sequence of SEQ ID NO: 138.
121. The isolated AAV particle of any one of embodiments 16 or 57-120, wherein [N5] is present immediately subsequent to position 595, and replaces positions 596-598 (e.g., V596, Q597, N598), numbered according to the amino acid sequence of SEQ ID NO: 138.
122. The isolated AAV particle of any one of embodiments 16 or 57-121, wherein [N5] corresponds to positions 603-605 of SEQ ID NO: 5, 8, or 3636.
123. The isolated AAV particle of any one of embodiments 16 or 57-122, wherein [N4]-[N5] corresponds to positions 600-605 of SEQ ID NO: 5, 8, or 3636.
124. The isolated AAV particle of any one of embodiments 16 or 57-123, wherein [N1]-[N2]-[N3]-[N4]-[N5] replaces positions 587-598 (e.g., A587, Q588, A589, Q590, A591, Q592, T593, G594, W595, V596, Q597, N598), numbered according to SEQ ID NO: 138.
125. The isolated AAV particle of any one of embodiments 16 or 57-124, wherein [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.
126. The isolated AAV particle of any one of embodiments 16 or 57-125, wherein [N1]-[N2]-[N3]-[N4]-[N5] corresponds to positions 587-605 of SEQ ID NO: 5, 8, or 3636.
127. The isolated AAV particle of any one of embodiments 15-126, wherein the AAV capsid variant comprises from N-terminus to C-terminus, [N1]-[N2].
128. The isolated AAV particle of any one of embodiments 16 or 30-127, wherein the AAV capsid variant comprises from N-terminus to C-terminus, [N1]-[N2]-[N3].
129. The isolated AAV particle of any one of embodiments 16 or 46-127, wherein the AAV capsid variant comprises from N-terminus to C-terminus, [N1]-[N2]-[N3]-[N4].
130. The isolated AAV particle of any one of embodiments 16 or 57-129, wherein the AAV capsid variant comprises from N-terminus to C-terminus, [N1]-[N2]-[N3]-[N4]-[N5].
131. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises [A][B], wherein [A] comprises the amino acid sequence of PLNGA (SEQ ID NO: 3679), and [B] comprises X1, X2, X3, X4, wherein:
   (i) X1 is: V, I, L, A, F, D, or G;
   (ii) X2 is: H, N, Q, P, D, L, R, or Y;
   (iii) X3 is: L, H, I, R, or V; and
   (iv) X4 is Y; and/or
   wherein the AAV capsid variant comprises an amino acid modification, e.g., a conservative substitution, of any of the aforesaid amino acids in (i)-(iv);
   optionally wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138; and/or
   (ii) one, two, or all of an amino acid other than V at position 596 (e.g., a D, F, A, E, L, G, or I), an amino acid other than Q at position 597 (e.g., P, K, R, L, H, or E), and/or an amino acid other than N at position 598 (e.g., H, S, T, P, K, I, D, or Y), relative to a reference sequence numbered according to the amino acid sequence of SEQ ID NO: 138.
132. The isolated AAV particle of embodiment 131, wherein:
   (i) X1 is V;
   (ii) X2 is H;
   (iii) X3 is L, H, or I; and
   (iv) X4 is Y.
133. The isolated AAV particle of embodiment 131 or 132, wherein [B] comprises:
   (i) VH, VN, VQ, IH, LH, VP, VD, AH, FH, DH, VL, GH, VR, VY, LY, HY, IY, RY, HL, HH, HI, NL, QL, PL, DL, HR, LL, RL, HV, or YL; or
   (ii) VH, LY, HY, IY, HL, HH, or HI.
134. The isolated AAV particle of any one of embodiments 131-133, wherein [B] comprises:
   (i) VHL, VHH, VHI, VNL, VQL, IHL, LHL, VPL, VDL, AHL, VHR, FHL, DHL, VLL, GHL, VRL, VHV, VYL, HLY, HHY, HIY, NLY, QLY, PLY, DLY, HRY, LLY, RLY, HVY, YLY;
   (ii) VHL, VHH, VHI, HLY, HHY, or HIY.
135. The isolated AAV particle of any one of embodiments 131-134, wherein [B] is or comprises:
   (i) VHLY (SEQ ID NO: 3741), VHHY (SEQ ID NO: 3747), VHIY (SEQ ID NO: 3746), VNLY (SEQ ID NO: 3744), VQLY (SEQ ID NO: 3751), IHLY(SEQ ID NO: 3752), LHLY (SEQ ID NO: 3749), VPLY(SEQ ID NO: 3743), VDLY (SEQ ID NO: 3753), AHLY (SEQ ID NO: 3754), VHRY (SEQ ID NO: 3745), FHLY (SEQ ID NO: 3748), DHLY (SEQ ID NO: 3750), VLLY (SEQ ID NO: 3755), GHLY (SEQ ID NO: 3756), VRLY (SEQ ID NO: 3757), VHVY (SEQ ID NO: 3742), or VYLY (SEQ ID NO: 3758) or a substitution, e.g., a conservative substitution, of any of the aforesaid amino acid sequences; or
   (ii) VHLY (SEQ ID NO: 3741), VHHY (SEQ ID NO: 3747), or VHIY (SEQ ID NO: 3746) or a substitution, e.g., a conservative substitution, of any of the aforesaid amino acid sequences.
136. The isolated AAV particle of any one of embodiments 131-135, wherein [B] is or comprises VHLY (SEQ ID NO: 3741).
137. The isolated AAV particle of any one of embodiments 131-135, wherein [A][B] is or comprises:

(i)
(SEQ ID NO: 3681)
PLNGAVH,
(SEQ ID NO: 1254)
PLNGAVN,
(SEQ ID NO: 1255)
PLNGAVQ,
(SEQ ID NO: 1256)
PLNGAIH,
(SEQ ID NO: 1257)
PLNGALH,
(SEQ ID NO: 1258)
PLNGAVP,
(SEQ ID NO: 1259)
PLNGAVD,
(SEQ ID NO: 1260)
PLNGAAH,
(SEQ ID NO: 1261)
PLNGAFH,
(SEQ ID NO: 1263)
PLNGADH,
(SEQ ID NO: 1264)
PLNGAVL,
(SEQ ID NO: 1265)
PLNGAGH,
(SEQ ID NO: 1266)
PLNGAVR,
or
(SEQ ID NO: 1267)
PLNGAVY;
or
(ii)
(SEQ ID NO: 3681)
PLNGAVH;

(iii) amino acid sequence comprising any portion of an amino acid sequence in (i) or (ii), e.g., any 2, 3, 4, 5, or 6 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
138. The isolated AAV particle of any one of embodiments 131-137, wherein [A][B] is or comprises:

(i)
(SEQ ID NO: 3648)
PLNGAVHLY,
(SEQ ID NO: 3810)
PLNGAVHHY,
(SEQ ID NO: 3808)
PLNGAVHIY,
(SEQ ID NO: 1268)
PLNGAVNLY,
(SEQ ID NO: 1269)
PLNGAVQLY,
(SEQ ID NO: 1270)
PLNGAIHLY,
(SEQ ID NO: 1271)
PLNGALHLY,
(SEQ ID NO: 1272)
PLNGAVPLY,
(SEQ ID NO: 1273)
PLNGAVDLY,
(SEQ ID NO: 1274)
PLNGAAHLY,
(SEQ ID NO: 1275)
PLNGAVHRY,
(SEQ ID NO: 1276)
PLNGAFHLY,
(SEQ ID NO: 1277)
PLNGADHLY,
(SEQ ID NO: 1278)
PLNGAVLLY,
(SEQ ID NO: 1279)
PLNGAGHLY,
(SEQ ID NO: 1280)
PLNGAVRLY,
(SEQ ID NO: 1281)
PLNGAVHVY,
or
(SEQ ID NO: 1282)
PLNGAVYLY;
(ii)
(SEQ ID NO: 3648)
PLNGAVHLY,
(SEQ ID NO: 3810)
PLNGAVHHY,
or
(SEQ ID NO: 3808)
PLNGAVHIY;

(iii) amino acid sequence comprising any portion of an amino acid sequence in (i) or (ii), e.g., any 2, 3, 4, 5, 6, 7, or 8 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
139. The isolated AAV particle of any one of embodiments 131-138, wherein [A][B] is or comprises PLNGAVHLY (SEQ ID NO: 3648).
140. The isolated AAV particle of any one of embodiments 131-138, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P).
141. The isolated AAV particle of any one of embodiments 131-140, which further comprises one, two, three, or all of an amino acid other than A at position 596 (e.g., D, S, or T), an amino acid other than Q at position 597 (e.g., K, H, L, P, or R), an amino acid other than A at position 598 (e.g., P or E), and/or an amino acid other than Q at position 599 (e.g., H, K, or P), relative to a reference sequence numbered according to the amino acid sequence of SEQ ID NO: 5, 8, or 3636.
142. The isolated AAV particle of any one of embodiments 131-141, which further comprises:
   (i) A at position 589, Q at position 590, A at position 591, and/or Q at position 592, numbered according to the amino acid sequence of SEQ ID NO: 138; or
   (ii) A at position 596, Q at position 597, A at position 598, and/or Q at position 599, numbered according to the amino acid sequence of SEQ ID NO: 5, 8, or 3636.
143. The isolated AAV particle of any one of embodiments 131-140, wherein the AAV capsid variant further comprises [C], wherein [C] comprises X4, X5, X6, and X7, wherein:
   (a) position X4 is: A, D, S, or T;
   (b) position X5 is: Q, K, H, L, P, or R;
   (c) position X6 is: A, P, or E; and
   (d) position X7 is: Q, H, K, or P; and/or
an amino acid modification, e.g., a conservative substitution, of any of the aforesaid amino acids in (a)-(d).
144. The isolated AAV particle of embodiment 143, wherein:
   (a) position X4 is: A, D, or S;
   (b) position X5 is Q or K;
   (c) position X6 is A or P; and/or
   (d) position X7 is Q.
145. The isolated AAV particle of embodiment 143 or 144, wherein [C] comprises:
   (i) AQ, AK, DQ, SQ, AH, AL, AP, AR, TQ, PQ, EQ, QA, QP, KA, HA, QE, LA, PA, or RA; or
   (ii) AQ, AK, DQ, SQ, PQ, QA, QP, or KA.
146. The isolated AAV particle of any one of embodiments 143-145, wherein [C] comprises:
   (i) AQA, AQP, AKA, DQA, SQA, AHA, AQE, ALA, APA, ARA, TQA, QAQ, QPQ, KAQ, HAQ, QEQ, QAK, LAQ, PAQ, RAQ, QAH, or QAP; or
   (ii) AQA, AQP, AKA, DQA, SQA, QAQ, QPQ, or KAQ.
147. The isolated AAV particle of any one of embodiments 143-146, wherein [C] is or comprises:
   (i) AQAQ (SEQ ID NO: 3759), AQPQ (SEQ ID NO: 3761), AKAQ (SEQ ID NO: 3763), DQAQ (SEQ ID NO: 3766), SQAQ (SEQ ID NO: 3760), AHAQ (SEQ ID NO: 3764), AQEQ (SEQ ID NO: 3770), AQAK (SEQ ID NO: 3768), ALAQ (SEQ ID NO: 3771), APAQ (SEQ ID NO: 3767), ARAQ SEQ ID NO: 3772), AQAH (SEQ ID NO: 3769), AQAP (SEQ ID NO: 3765), or TQAQ (SEQ ID NO: 3773), or a substitution, e.g., a conservative substitution, of any of the aforesaid amino acid sequences; or
   (ii) AQAQ (SEQ ID NO: 3759), AQPQ (SEQ ID NO: 3761), AKAQ (SEQ ID NO: 3763), DQAQ (SEQ ID NO: 3766), or SQAQ (SEQ ID NO: 3760).
148. The isolated AAV particle of any one of embodiments 143-147, wherein [C] is or comprises AQAQ (SEQ ID NO: 3759).
149. The isolated AAV particle of any one of embodiments 143-148, wherein [B][C] is or comprises:

(i)
(SEQ ID NO: 3811)
VHLYAQAQ,
(SEQ ID NO: 3818)
VHHYAQAQ,
(SEQ ID NO: 3812)
VHLYAQPQ,
(SEQ ID NO: 3814)
VHLYAKAQ,
(SEQ ID NO: 3815)
VHLYDQAQ,
(SEQ ID NO: 3813)
VHLYSQAQ,
(SEQ ID NO: 3816)
VHIYAQAQ,
(SEQ ID NO: 1283)
VHLYAHAQ,
(SEQ ID NO: 1284)
VNLYAQAQ,
(SEQ ID NO: 1285)
VQLYAQAQ,
(SEQ ID NO: 1286)
VHLYAQEQ,
(SEQ ID NO: 1287)
IHLYAQAQ,
(SEQ ID NO: 1288)
LHLYAQAQ,
(SEQ ID NO: 1289)
VPLYAQAQ,
(SEQ ID NO: 1290)
VHLYAQAK,
(SEQ ID NO: 1291)
VDLYAQAQ,
(SEQ ID NO: 1292)
AHLYAQAQ,
(SEQ ID NO: 1293)
VHRYAQAQ,
(SEQ ID NO: 1294)
FHLYAQAQ,
(SEQ ID NO: 1295)
VHLYALAQ,
(SEQ ID NO: 1296)
DHLYAQAQ,
(SEQ ID NO: 1297)
VHLYAPAQ,
(SEQ ID NO: 1298)
VHLYARAQ,
(SEQ ID NO: 1299)
VHLYAQAH,
(SEQ ID NO: 1300)
VLLYAQAQ,
(SEQ ID NO: 1301)
VHLYAQAP,
(SEQ ID NO: 1302)
GHLYAQAQ,
(SEQ ID NO: 1303)
VRLYAQAQ,
(SEQ ID NO: 3817)
VHVYAQAQ,
(SEQ ID NO: 1304)
VYLYAQAQ,
(SEQ ID NO: 1305)
VHLYTQAQ;
(ii)
(SEQ ID NO: 3811)
VHLYAQAQ,
(SEQ ID NO: 3818)
VHHYAQAQ,
(SEQ ID NO: 3812)
VHLYAQPQ,
(SEQ ID NO: 3814)
VHLYAKAQ,
(SEQ ID NO: 3815)
VHLYDQAQ,
(SEQ ID NO: 3813)
VHLYSQAQ,
(SEQ ID NO: 3816)
VHIYAQAQ;

(iii) amino acid sequence comprising any portion of an amino acid sequence in (i) or (ii), e.g., any 2, 3, 4, 5, 6, or 7 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
150. The isolated AAV particle of any one of embodiments 143-149, wherein [B][C] is or comprises VHLYAQAQ (SEQ ID NO: 3811).
151. The isolated AAV particle of any one of embodiments 143-150, wherein [A][B][C] comprises:

(i)
(SEQ ID NO: 3840)
PLNGAVHLYAQ,
PLNGAVHHYAQ,
(SEQ ID NO: 3826)
PLNGAVHLYAK,
(SEQ ID NO: 3828)
PLNGAVHLYDQ,
(SEQ ID NO: 3829)
PLNGAVHLYSQ,
(SEQ ID NO: 3838)
PLNGAVHIYAQ,
(SEQ ID NO: 1306)
PLNGAVHLYAH,
(SEQ ID NO: 1307)
PLNGAVNLYAQ,
(SEQ ID NO: 1308)
PLNGAVQLYAQ,
(SEQ ID NO: 1309)
PLNGAIHLYAQ,
(SEQ ID NO: 1310)
PLNGALHLYAQ,
(SEQ ID NO: 1311)
PLNGAVPLYAQ,
(SEQ ID NO: 1312)
PLNGAVDLYAQ,
(SEQ ID NO: 1313)
PLNGAAHLYAQ,
(SEQ ID NO: 1314)
PLNGAVHRYAQ,
(SEQ ID NO: 1315)
PLNGAFHLYAQ,
(SEQ ID NO: 1316)
PLNGAVHLYAL,
(SEQ ID NO: 1317)
PLNGADHLYAQ,
(SEQ ID NO: 1318)
PLNGAVHLYAP,
(SEQ ID NO: 1319)
PLNGAVHLYAR,
(SEQ ID NO: 1320)
PLNGAVLLYAQ,
(SEQ ID NO: 1321)
PLNGAGHLYAQ,
(SEQ ID NO: 1322)
PLNGAVRLYAQ,
(SEQ ID NO: 1323)
PLNGAVHVYAQ,
(SEQ ID NO: 1324)
PLNGAVYLYAQ,
(SEQ ID NO: 1325)
PLNGAVHLYTQ,
(ii)
(SEQ ID NO: 3827)
PLNGAVHLYAQ,
(SEQ ID NO: 3840)
PLNGAVHHYAQ,
(SEQ ID NO: 3826)
PLNGAVHLYAK,
(SEQ ID NO: 3828)
PLNGAVHLYDQ,
(SEQ ID NO: 3829)
PLNGAVHLYSQ,
(SEQ ID NO: 3838)
PLNGAVHIYAQ;

(iii) amino acid sequence comprising any portion of an amino acid sequence in (i) or (ii), e.g., any 2, 3, 4, 5, 6, 7, 8, 9, or 10 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
152. The isolated AAV particle of any one of embodiments 143-151, wherein [A][B][C] is or comprises:

(i)
(SEQ ID NO: 3850)
PLNGAVHLYAQAQ,
(SEQ ID NO: 3864)
PLNGAVHHYAQAQ,
(SEQ ID NO: 3851)
PLNGAVHLYAQPQ,
(SEQ ID NO: 3849)
PLNGAVHLYAKAQ,
(SEQ ID NO: 3852)
PLNGAVHLYDQAQ,
(SEQ ID NO: 3853)
PLNGAVHLYSQAQ,
(SEQ ID NO: 3862)
PLNGAVHIYAQAQ,
(SEQ ID NO: 1326)
PLNGAVHLYAHAQ,
(SEQ ID NO: 1327)
PLNGAVNLYAQAQ,
(SEQ ID NO: 1328)
PLNGAVQLYAQAQ,
(SEQ ID NO: 1329)
PLNGAVHLYAQEQ,
(SEQ ID NO: 1330)
PLNGAIHLYAQAQ,
(SEQ ID NO: 1331)
PLNGALHLYAQAQ,
(SEQ ID NO: 1332)
PLNGAVPLYAQAQ,
(SEQ ID NO: 1333)
PLNGAVHLYAQAK,
(SEQ ID NO: 1334)
PLNGAVDLYAQAQ,
(SEQ ID NO: 1335)
PLNGAAHLYAQAQ,
(SEQ ID NO: 1336)
PLNGAVHRYAQAQ,
(SEQ ID NO: 1337)
PLNGAFHLYAQAQ,
(SEQ ID NO: 1338)
PLNGAVHLYALAQ,
(SEQ ID NO: 1339)
PLNGADHLYAQAQ,
(SEQ ID NO: 1340)
PLNGAVHLYAPAQ,
(SEQ ID NO: 1341)
PLNGAVHLYARAQ,
(SEQ ID NO: 1342)
PLNGAVHLYAQAH,
(SEQ ID NO: 1343)
PLNGAVLLYAQAQ,
(SEQ ID NO: 1344)
PLNGAVHLYAQAP,
(SEQ ID NO: 1345)
PLNGAGHLYAQAQ,
(SEQ ID NO: 1346)
PLNGAVRLYAQAQ,
(SEQ ID NO: 1347)
PLNGAVHVYAQAQ,
(SEQ ID NO: 1348)
PLNGAVYLYAQAQ,
(SEQ ID NO: 1349)
PLNGAVHLYTQAQ,
(ii)
(SEQ ID NO: 3850)
PLNGAVHLYAQAQ,
(SEQ ID NO: 3864)
PLNGAVHHYAQAQ,
(SEQ ID NO: 3851)
PLNGAVHLYAQPQ,
(SEQ ID NO: 3849)
PLNGAVHLYAKAQ,
(SEQ ID NO: 3852)
PLNGAVHLYDQAQ,
(SEQ ID NO: 3853)
PLNGAVHLYSQAQ,
(SEQ ID NO: 3862)
PLNGAVHIYAQAQ;

(iii) amino acid sequence comprising any portion of an amino acid sequence in (i) or (ii), e.g., any 2, 3, 4, 5, 6, 7, 8, 9, 10, 11, or 12 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
153. The isolated AAV particle of any one of embodiments 143-152, wherein [A][B][C] is or comprises PLNGAVHLYAQAQ (SEQ ID NO: 3850).
154. The isolated AAV particle of any one of embodiment 131-153, wherein the AAV capsid variant further comprises one, two, or all of an amino acid other than T at position 593 (e.g., a V, S, L, R, I, A, N, C, Q, M, P, or K), an amino acid other than G at position 594 (e.g., T, M, A, K, S, Q, V, I, R, N, P, L, H, or Y), and/or an amino acid other than W at position 595 (e.g., K, Q, S, P, C, A, G, N, T, R, V, M, H, L, E, F, or Y), relative to a reference sequence numbered according to the amino acid sequence of SEQ ID NO: 138.
155. The isolated AAV particle of any one of embodiment 131-154, which further comprises one, two, or all of an amino acid other than T at position 600 (e.g., a V, S, L, R, I, A, N, C, Q, M, P, or K), an amino acid other than G at position 601 (e.g., T, M, A, K, S, Q, V, I, R, N, P, L, H, or Y), and/or an amino acid other than W at position 602 (e.g., K, Q, S, P, C, A, G, N, T, R, V, M, H, L, E, F, or Y), relative to a reference sequence numbered according to the amino acid sequence of SEQ ID NO: 5, 8, 3636.
156. The isolated AAV particle of any one of embodiment 131-155, which further comprises one, two, three or all of:
   (i) the amino acid V, S, L, R, I, A, N, C, Q, M, P, or K (e.g., L) at position 593 numbered according to SEQ ID NO: 138 or at position 600 numbered according to SEQ ID NO: or 3636;
   (ii) the amino acid T, M, A, K, S, Q, V, I, R, N, P, L, H, or Y (e.g., S) at position 594 numbered according to SEQ ID NO: 138, or at position 601 numbered according to SEQ ID NO: 5, 8, or 3636; and/or
   (iii) the amino acid K, Q, S, P, C, A, G, N, T, R, V, M, H, L, E, F, or Y (e.g. P) at position 595 numbered according to SEQ ID NO: 138 or at position 602 numbered according to SEQ ID NO: 5, 8, or 3636).
157. The isolated AAV particle of any one of embodiment 131-155, which further comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138, or at position 601 numbered according to SEQ ID NO: 5, 8, or 3636; and
   (iii) the amino acid P at position 595 numbered according to SEQ ID NO: 138 or at position 602 numbered according to SEQ ID NO: 5, 8, or 3636).
158. The isolated AAV particle of any one of embodiment 131-152, which further comprises:
   (i) T at position 593, G at position 594, and/or W at position 595, numbered according to SEQ ID NO: 138;
   (ii) T at position 600, G at position 601, and/or W at position 602, numbered according to SEQ ID NO: 5, 8, or 3636.
159. The isolated AAV particle of any one of embodiments 131-158, wherein the AAV capsid variant further comprises [D], wherein [D] comprises X8, X9, and X10, wherein:
   (a) position X8 is: T, V, S, L, R, I, A, N, C, Q, M, P, or K;
   (b) position X9 is: T, M, A, G, K, S, Q, V, I, R, N, P, L, H, or Y; and
   (c) position X10 is: K, Q, W, S, P, C, A, G, N, T, R, V, M, H, L, E, F, or Y; and/or
   an amino acid modification, e.g., a conservative substitution, of any of the aforesaid amino acids in (a)-(c).
160. The isolated AAV particle of embodiment 159, wherein:
   (a) position X8 is: T, V, S, L, R, I, A, N, C, Q, or M;
   (b) position X9 is: T, M, A, G, K, S, Q, V, I, R, N, P, L, or H; and/or
   (c) position X10 is: K, Q, W, S, P, C, A, G, N, T, R, V, M, H, L, or E.
161. The isolated AAV particle of embodiment 159 or 160, wherein [D] is or comprises:
   (i) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, PG, QS, RY, SQ, NQ, LL, LP, AQ, TY, NL, SP, LV, KG, VP, AV, KS, AM, SL, AL, RP, IP, MK, AW, GS, KQ, AP, SK, AK, GC, QK, MQ, QP, GP, QQ, AN, GK, QR, PP, AR, GG, MS, NP, KP, MN, KA, SN, MP, HP, GN, RW, MT, SR, GW, QH, GL, QM, VW, MG, AH, QT, GR, SH, GQ, GT, GA, NG, QN, VE, MM, QL, QG, YS, GM, LR, AF, PQ, SW, QW, YA, ML, GF, PA, PS, PT, GY, GV, PW, PR; or
   (ii) TT, TM, VA, TA, TG, VK, SA, LS, LA, TQ, TV, RI, RA, LT, ST, TS, VS, VT, RQ, IS, VR, LG, TN, VQ, AA, RS, IQ, IA, RG, NS, LQ, VM, SM, VG, CS, TP, SS, AG, TL, LN, TK, CT, AS, LK, LM, LH, RT, RM, VH, TR, SG, VL, QA, NA, AT, NT, RL, IT, IG, RN, NM, NV, MA, IL, VN, SV, RV, MK, AQ, AW, GS, KQ, AP, SK, AK, GC, QK, SP, MQ, SQ, QP, RP, GP, NQ, QQ, AN, GK, QS, QR, PP, AR, GG, MS, NP, KP, MN, KS, KA, SN, MP, HP, GN, RW, MT, AM, SR, GW, QH, GL, AV, QM, VW, MG, AL, AH, SL, QT, GR, SH, LP, GQ, GT, GA, NG, QN, IP, or VE.
162. The isolated AAV particle of any one of embodiments 159-161, wherein [D] is or comprises:
   (i) TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG, or a substitution, e.g., a conservative substitution, of any of the aforesaid amino acid sequences; or
   (ii) TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS.
163. The isolated AAV particle of any one of embodiments 159-162, wherein [D] is or comprises TGW.
164. The isolated AAV particle of any one of embodiments 159-163, wherein [D] is or comprises LSP.
165. The isolated AAV particle of any one of embodiments 131-164, wherein the AAV capsid variant further comprises one, two, or all of an amino acid other than V at position 596 (e.g., D, F, A, E, L, G, or I), an amino acid other than Q at position 597 (e.g., R, P, K, L, H, or E), and/or an amino acid other than N at position 598 (e.g., H, S, T, P, K, I, D, or Y), relative to a reference sequence numbered according to the amino acid sequence of SEQ ID NO: 138.
166. The isolated AAV particle of any one of embodiments 131-165, which further comprises one, two, or all of an amino acid other than V at position 603 (e.g., D, F, A, E, L, G, or I), an amino acid other than Q at position 604 (e.g., R, P, K, L, H, or E), and/or an amino acid other than N at position 605 (e.g., H, S, T, P, K, I, D, or Y), relative to a reference sequence numbered according to the amino acid sequence of SEQ ID NO: 5, 8, or 3636.
167. The isolated AAV particle of any one of embodiments 131-166, which further comprises one, two, or all of:
   (i) the amino acid D, F, A, E, L, G, or I at position 596, numbered according to SEQ ID NO: 138, or at position 603 numbered according to SEQ ID NO: 5, 8, or 3636;
   (ii) the amino acid R, P, K, L, H, or E at position 597, numbered according to SEQ ID NO: 138, or at position 604 numbered according to SEQ ID NO: 5, 8, or 3636; and/or
   (iii) the amino acid H, S, T, P, K, I, D, or Y at position 598, numbered according to SEQ ID NO: 138, or at position 605 numbered according to SEQ ID NO: 5, 8, or 3636.
168. The isolated AAV particle of any one of embodiments 131-167, which further comprises P, K, E, or H at position 597, numbered according to SEQ ID NO: 138.
169. The isolated AAV particle of any one of embodiments 131-168, which further comprises one, two, or all of:
   (i) the amino acid V at position 596 numbered according to SEQ ID NO: 138 or at position 603 numbered according to SEQ ID NO: 5, 8, or 3636;
   (ii) the amino acid K, P, E or H at position 597 numbered according to SEQ ID NO: 138 or at position 604 numbered according to SEQ ID NO: 5, 8, or 3636; and
   (iii) the amino acid N at position 598 numbered according to the amino acid sequence of SEQ ID NO: 138 or at position 605 numbered according to SEQ ID NO: 5, 8, or 3636.
170. The isolated AAV particle of any one of embodiments 131-169, wherein the AAV capsid variant further comprises [E], wherein [E] comprises X11, X12, and X13, wherein:
   (a) position X11 is: V, D, F, A, E, L, G, or I;
   (b) position X12 is: Q, R, P, K, L, H, or E; and
   (c) position X13 is: N, H, S, T, P, K, I, D, or Y; or
an amino acid modification, e.g., a conservative substitution, of any of the aforesaid amino acids in (a)-(c).
171. The isolated AAV particle of embodiment 170, wherein:
   (a) position X11 is: V, D, F, A, E, L, or G;
   (b) position X12 is: Q, R, P, K, or L; and/or
   (c) position X13 is: N, H, S, T, P, K, I, or D.
172 The isolated AAV particle of embodiment 170 or 171, wherein:
   (a) position X11 is V;
   (b) position X12: Q, R, P, K, or L; and/or
   (c) position X13 is: N.
173. The isolated AAV particle of any one of embodiments 170-172, wherein position X12 is P.
174. The isolated AAV particle of any one of embodiments 170-172, wherein position X12 is K.
175. The isolated AAV particle of any one of embodiments 170-172, wherein position X12 is E or H.
176. The isolated AAV particle of any one of embodiment 170-175, wherein [E] comprises:
   (i) VQ, DQ, FQ, VR, VP, VK, AQ, EQ, LQ, GQ, VL, VH, VE, DK, GH, IQ, QN, QH, QS, QT, QP, RN, PN, KN, QK, QI, LN, QD, HN, KT, KK, EN, QY, or PH; or
   (ii) VQ, DQ, FQ, VR, VP, VK, AQ, EQ, LQ, GQ, VL, QN, QH, QS, QT, QP, RN, PN, KN, QK, QI, LN, or QD.
177. The isolated AAV particle of any one of embodiments 170-176, wherein [E] is or comprises:
   (i) VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH; or
   (ii) VQN, DQN, VQH, FQN, VQS, VQT, VQP, VRN, VPN, VKN, AQN, VQK, EQN, VQI, LQN, GQT, VLN, or VQD.
178. The isolated AAV particle of any one of embodiments 170-177, wherein [E] is or comprises VQN, VPN, or VKN.
179. The isolated AAV particle of any one of embodiments 170-178, wherein [E] is or comprises VEN, VHN, VKN, or VPN.
180. The isolated AAV particle of any one of embodiments 170-179, wherein [D][E] is or comprises:

(i)
(SEQ ID NO: 1192)
TTKVQN,
(SEQ ID NO: 1158)
TMKVQN,
(SEQ ID NO: 1207)
VAQVQN,
(SEQ ID NO: 3995)
TAWDQN,
(SEQ ID NO: 3698)
TGSVQH,
(SEQ ID NO: 1217)
VKQVQN,
(SEQ ID NO: 3868)
SAPVQN,
(SEQ ID NO: 3929)
LSKVQN,
(SEQ ID NO: 3905)
LAPVQN,
(SEQ ID NO: 3907)
LAQVQN,
(SEQ ID NO: 3985)
TAKVON,
(SEQ ID NO: 3972)
SAKVQN,
(SEQ ID NO: 3999)
TGCFQN,
(SEQ ID NO: 1171)
TQKVQN,
(SEQ ID NO: 1197)
TVAVQN,
(SEQ ID NO: 3931)
LSPVQN,
(SEQ ID NO: 1195)
TTQVQN,
(SEQ ID NO: 3990)
TAQVQN,
(SEQ ID NO: 3951)
RIAVQN,
(SEQ ID NO: 3948)
RASVQN,
(SEQ ID NO: 1193)
TTPVQN,
(SEQ ID NO: 3867)
LTPVQN,
(SEQ ID NO: 3980)
STPVQN,
(SEQ ID NO: 3875)
TSPVQN,
(SEQ ID NO: 1161)
TMQVQN,
(SEQ ID NO: 1181)
TSKVQN,
(SEQ ID NO: 1237)
VSQVQN,
(SEQ ID NO: 3882)
VSPVQN,
(SEQ ID NO: 1200)
TVQVQN,
(SEQ ID NO: 1242)
VTAVQN,
(SEQ ID NO: 3958)
RQPVQN,
(SEQ ID NO: 3898)
ISGVQN,
(SEQ ID NO: 1232)
VRPVQN,
(SEQ ID NO: 3911)
LGPVQN,
(SEQ ID NO: 1167)
TNQVQN,
(SEQ ID NO: 1229)
VQQVQN,
(SEQ ID NO: 1206)
VANVQN,
(SEQ ID NO: 3884)
AAPVQN,
(SEQ ID NO: 3964)
RSTVQN,
(SEQ ID NO: 1156)
TMAVQN,
(SEQ ID NO: 3896)
IQPVQN,
(SEQ ID NO: 3892)
IASVQN,
(SEQ ID NO: 3881)
TVSVQN,
(SEQ ID NO: 3950)
RGSVQN,
(SEQ ID NO: 3942)
NSPVQN,
(SEQ ID NO: 3925)
LQPVQN,
(SEQ ID NO: 1243)
VTGVQN,
(SEQ ID NO: 1221)
VMQVQN,
(SEQ ID NO: 3976)
SMAVQN,
(SEQ ID NO: 1211)
VGKVQN,
(SEQ ID NO: 3897)
IQSVQN,
(SEQ ID NO: 3888)
CSPVQN,
(SEQ ID NO: 3927)
LQRVQN,
(SEQ ID NO: 3996)
TAWVQH,
(SEQ ID NO: 1169)
TPPVQN,
(SEQ ID NO: 1244)
VTKVQN,
(SEQ ID NO: 3869)
SSPVQN,
(SEQ ID NO: 3885)
AGPVQN,
(SEQ ID NO: 3908)
LARVQN,
(SEQ ID NO: 1196)
TTTVQN,
(SEQ ID NO: 3673)
TGGFQN,
(SEQ ID NO: 1155)
TLQVQN,
(SEQ ID NO: 1163)
TMSVQN,
(SEQ ID NO: 1205)
VAKVQN,
(SEQ ID NO: 3946)
RAAVQN,
(SEQ ID NO: 1199)
TVGVQN,
(SEQ ID NO: 3924)
LNPVQN,
(SEQ ID NO: 3932)
LSQVQN,
(SEQ ID NO: 1151)
TKPVQN,
(SEQ ID NO: 1166)
TNAVQN,
(SEQ ID NO: 3910)
LATVQN,
(SEQ ID NO: 1245)
VTPVQN,
(SEQ ID NO: 1226)
VQAVQN,
(SEQ ID NO: 3874)
TTSVQN,
(SEQ ID NO: 3889)
CTPVQN,
(SEQ ID NO: 3984)
TAGVQN,
(SEQ ID NO: 1185)
TSQVQN,
(SEQ ID NO: 1159)
TMNVQN,
(SEQ ID NO: 1188)
TSTVQN,
(SEQ ID NO: 1216)
VKPVQN,
(SEQ ID NO: 3886)
ASPVQN,
(SEQ ID NO: 1203)
VAAVQN,
(SEQ ID NO: 3915)
LKSVQN,
(SEQ ID NO: 3890)
IAAVQN,
(SEQ ID NO: 3983)
TAAVQN,
(SEQ ID NO: 1150)
TKAVQN,
(SEQ ID NO: 1139)
TGSVQS,
(SEQ ID NO: 1235)
VSNVQN,
(SEQ ID NO: 3988)
TAPVQN,
(SEQ ID NO: 3921)
LMPVQN,
(SEQ ID NO: 3914)
LHPVQN,
(SEQ ID NO: 3947)
RAQVQN,
(SEQ ID NO: 3936)
LTNVQN,
(SEQ ID NO: 3969)
RTTVQN,
(SEQ ID NO: 1189)
TSVVQN,
(SEQ ID NO: 3956)
RMSVQN,
(SEQ ID NO: 1212)
VGNVQN,
(SEQ ID NO: 3922)
LMQVQN,
(SEQ ID NO: 3993)
TATVQN,
(SEQ ID NO: 1214)
VHPVQN,
(SEQ ID NO: 1236)
VSPVQT,
(SEQ ID NO: 3900)
ISSVQN,
(SEQ ID NO: 1208)
VASVQN,
(SEQ ID NO: 1177)
TRWDQN,
(SEQ ID NO: 1165)
TMTVQN,
(SEQ ID NO: 3963)
RSSVQN,
(SEQ ID NO: 3877)
TAWVQN,
(SEQ ID NO: 3966)
RTGVQN,
(SEQ ID NO: 1209)
VATVQN,
(SEQ ID NO: 1246)
VTSVQN,
(SEQ ID NO: 3883)
VSSVQN,
(SEQ ID NO: 1168)
TNSVQN,
(SEQ ID NO: 1215)
VKAVQN,
(SEQ ID NO: 3975)
SGPVQN,
(SEQ ID NO: 3696)
TGPVQN,
(SEQ ID NO: 3986)
TAMVQN,
(SEQ ID NO: 1174)
TQPVQN,
(SEQ ID NO: 1175)
TQQVQN,
(SEQ ID NO: 1238)
VSRVQN,
(SEQ ID NO: 1147)
TGWVQP,
(SEQ ID NO: 1233)
VSAVQN,
(SEQ ID NO: 1219)
VLSVQN,
(SEQ ID NO: 1170)
TQHVQN,
(SEQ ID NO: 3909)
LASVQN,
(SEQ ID NO: 3945)
QAPVQN,
(SEQ ID NO: 3939)
NAQVQN,
(SEQ ID NO: 3887)
ATPVQN,
(SEQ ID NO: 1228)
VQPVQN,
(SEQ ID NO: 1190)
TTAVQN,
(SEQ ID NO: 1149)
TGWVRN,
(SEQ ID NO: 3866)
LAAVQN,
(SEQ ID NO: 1184)
TSPDQN,
(SEQ ID NO: 3960)
RSGVQN,
(SEQ ID NO: 3677)
TGGVQT,
(SEQ ID NO: 1145)
TGWVPN,
(SEQ ID NO: 1194)
TTPVQT,
(SEQ ID NO: 3916)
LMAVQN,
(SEQ ID NO: 1160)
TMPVQN,
(SEQ ID NO: 3904)
LANVQN,
(SEQ ID NO: 1241)
VSTVQN,
(SEQ ID NO: 3974)
SAQVQN,
(SEQ ID NO: 3943)
NTPVQN,
(SEQ ID NO: 1240)
VSSVQT,
(SEQ ID NO: 1201)
TVSVKN,
(SEQ ID NO: 1247)
TGLVQN,
(SEQ ID NO: 3879)
TGSVQN,
(SEQ ID NO: 3672)
TGGAQN,
(SEQ ID NO: 3994)
TAVVQN,
(SEQ ID NO: 3954)
RLGVQN,
(SEQ ID NO: 3967)
RTLVQN,
(SEQ ID NO: 1172)
TQMVQN,
(SEQ ID NO: 3901)
ITPVQN,
(SEQ ID NO: 1202)
TVWVQK,
(SEQ ID NO: 3959)
RSAVQN,
(SEQ ID NO: 3991)
TASVQN,
(SEQ ID NO: 1157)
TMGVQN,
(SEQ ID NO: 3675)
TGGVQH,
(SEQ ID NO: 1230)
VQSVQN,
(SEQ ID NO: 3878)
TGGVQN,
(SEQ ID NO: 3899)
ISPVQN,
(SEQ ID NO: 1248)
TGWVKN,
(SEQ ID NO: 3697)
TGSAQN,
(SEQ ID NO: 3871)
TGWAQN,
(SEQ ID NO: 1164)
TMSVQT,
(SEQ ID NO: 1210)
VGGVQN,
(SEQ ID NO: 3906)
LAPVQT,
(SEQ ID NO: 3876)
TALVQN,
(SEQ ID NO: 3902)
LAGVQN,
(SEQ ID NO: 3965)
RTAVQN,
(SEQ ID NO: 3961)
RSPVQN,
(SEQ ID NO: 3873)
TLAVQN,
(SEQ ID NO: 3903)
LAHVQN,
(SEQ ID NO: 1182)
TSLVQN,
(SEQ ID NO: 3955)
RLSVQN,
(SEQ ID NO: 3978)
LMGVQN,
SMQVQN,
(SEQ ID NO: 1176)
TQTVQN,
(SEQ ID NO: 1142)
TGWEQN,
(SEQ ID NO: 1213)
VGSVQN,
(SEQ ID NO: 1234)
VSGVQN,
(SEQ ID NO: 1220)
VMAVQN,
(SEQ ID NO: 3893)
IGGVQN,
(SEQ ID NO: 3891)
IAGVQN,
(SEQ ID NO: 3870)
TGRVQN,
(SEQ ID NO: 3928)
LSHVQN,
(SEQ ID NO: 1231)
VQTVQN,
(SEQ ID NO: 3880)
TGWDQN,
(SEQ ID NO: 3957)
RNSVQN,
(SEQ ID NO: 1154)
TLPVQN,
(SEQ ID NO: 1152)
TKQVQN,
(SEQ ID NO: 3912)
LGQVQN,
(SEQ ID NO: 3941)
NMQVQN,
(SEQ ID NO: 3944)
NVQVQN,
(SEQ ID NO: 3699)
TGSVQI,
(SEQ ID NO: 3949)
RGGVQN,
(SEQ ID NO: 1222)
VMSVQN,
(SEQ ID NO: 1191)
TTGVQN,
(SEQ ID NO: 3930)
LSPVQK,
(SEQ ID NO: 3933)
LSRVQN,
(SEQ ID NO: 1239)
VSSVQK,
(SEQ ID NO: 3989)
TAPVQT,
(SEQ ID NO: 3938)
MAPVQN,
(SEQ ID NO: 3895)
ILGVQN,
(SEQ ID NO: 3992)
TASVQT,
(SEQ ID NO: 3697)
TGSLQN,
(SEQ ID NO: 1141)
TGTVQN,
(SEQ ID NO: 1140)
TGSVQT,
(SEQ ID NO: 1187)
TSSVQT,
(SEQ ID NO: 1180)
TSHVQN,
(SEQ ID NO: 3952)
RIGVQN,
(SEQ ID NO: 1144)
TGWGQT,
(SEQ ID NO: 3973)
SAMVQN,
(SEQ ID NO: 1183)
TSMVQN,
(SEQ ID NO: 3977)
SMGVQN,
(SEQ ID NO: 3979)
SMSVQN,
(SEQ ID NO: 1186)
TSSVQN,
(SEQ ID NO: 1179)
TSGVQN,
(SEQ ID NO: 3998)
TGAVQN,
(SEQ ID NO: 1225)
VNSVQN,
(SEQ ID NO: 1204)
VAGVQN,
(SEQ ID NO: 3894)
IGSVQN,
(SEQ ID NO: 3913)
LGSVQN,
(SEQ ID NO: 1224)
VNGVQN,
(SEQ ID NO: 3935)
LTAVQN,
(SEQ ID NO: 1227)
VQNVQN,
(SEQ ID NO: 1153)
TKSVQN,
(SEQ ID NO: 3982)
SVGVQN,
(SEQ ID NO: 3997)
TAWVQT,
(SEQ ID NO: 3940)
NASVQN,
(SEQ ID NO: 1178)
TSAVQN,
(SEQ ID NO: 1162)
TMSVKN,
(SEQ ID NO: 3987)
TANVQN,
(SEQ ID NO: 1143)
TGWFQN,
(SEQ ID NO: 3674)
TGGVLN,
(SEQ ID NO: 3937)
LTSVQN,
(SEQ ID NO: 1148)
TGWVQT,
(SEQ ID NO: 3962)
RSQVQN,
(SEQ ID NO: 3953)
RIPVQN,
(SEQ ID NO: 1146)
TGWVQD,
(SEQ ID NO: 3970)
RVEVQN,
(SEQ ID NO: 1218)
VLPVQN,
(SEQ ID NO: 3676)
TGGVQK,
(SEQ ID NO: 3981)
SVAVQN,
(SEQ ID NO: 3926)
LQQVQN,
(SEQ ID NO: 3934)
LSTVQN,
(SEQ ID NO: 3971)
SAAVQN,
(SEQ ID NO: 3968)
RTSVQN,
(SEQ ID NO: 1173)
TQNVQN,
(SEQ ID NO: 1223)
VNAVQN,
(SEQ ID NO: 1198)
TVAVQT,
(SEQ ID NO: 3923)
LMSVQN,
(SEQ ID NO: 3865)
TGWVQN,
(SEQ ID NO: 1250)
TGWVQH,
(SEQ ID NO: 3872)
TGWVQS,
(SEQ ID NO: 1350)
TMMVQN,
(SEQ ID NO: 1351)
TGGVQS,
(SEQ ID NO: 1352)
TGSFQN,
(SEQ ID NO: 1353)
RSVVQN,
(SEQ ID NO: 1354)
TGSVQK,
(SEQ ID NO: 1355)
TQLVQN,
(SEQ ID NO: 1356)
TGGVHN,
(SEQ ID NO: 1357)
RTPVQN,
(SEQ ID NO: 1358)
RQQVQN,
(SEQ ID NO: 1359)
TGSVRN,
(SEQ ID NO: 1360)
VQGVQN,
(SEQ ID NO: 1361)
PGWVQT,
(SEQ ID NO: 1362)
STQVQN,
(SEQ ID NO: 1363)
QSPVQN,
(SEQ ID NO: 1364)
RYSVQN,
(SEQ ID NO: 1365)
TQRVQN,
(SEQ ID NO: 1366)
SAGVQN,
(SEQ ID NO: 1367)
SAPVQT,
(SEQ ID NO: 1368)
RQSVQN,
(SEQ ID NO: 1369)
SQPVQN,
(SEQ ID NO: 1370)
VASVKN,
(SEQ ID NO: 1371)
TAWVRN,
(SEQ ID NO: 1372)
TGGGQN,
(SEQ ID NO: 1373)
STSVQN,
(SEQ ID NO: 1374)
VLGVQN,
(SEQ ID NO: 1375)
NQPVQN,
(SEQ ID NO: 1376)
LGTVQN,
(SEQ ID NO: 1377)
RAGVQN,
(SEQ ID NO: 1378)
TGGVKN,
(SEQ ID NO: 1379)
TAWLQN,
(SEQ ID NO: 1380)
TRWVQK,
(SEQ ID NO: 1381)
LAPVKN,
(SEQ ID NO: 1382)
TGSVQD,
(SEQ ID NO: 1383)
TGMVQN,
(SEQ ID NO: 1384)
LSNVQN,
(SEQ ID NO: 1385)
RLPVQN,
(SEQ ID NO: 1386)
RQGVQN,
(SEQ ID NO: 1387)
STPVQT,
(SEQ ID NO: 1388)
TTPVKN,
(SEQ ID NO: 1389)
RLTVQN,
(SEQ ID NO: 1390)
TLRVQN,
(SEQ ID NO: 1391)
SAFVQN,
(SEQ ID NO: 1392)
SVQVQN,
(SEQ ID NO: 1393)
LLPVQN,
(SEQ ID NO: 1394)
RTQVQN,
(SEQ ID NO: 1395)
TGSDQN,
(SEQ ID NO: 1396)
VASDQN,
(SEQ ID NO: 1397)
VSPVKN,
(SEQ ID NO: 1398)
LPPVQN,
(SEQ ID NO: 1399)
SSPVQT,
(SEQ ID NO: 1400)
AQPVQN,
(SEQ ID NO: 1401)
TPQVQN,
(SEQ ID NO: 1402)
TSWVQN,
(SEQ ID NO: 1403)
TGGDQN,
(SEQ ID NO: 1404)
LSPVKN,
(SEQ ID NO: 1405)
SSPVKN,
(SEQ ID NO: 1406)
NTTVQN,
(SEQ ID NO: 1407)
TTRVQN,
(SEQ ID NO: 1408)
TQWVQN,
(SEQ ID NO: 1409)
TGSVHN,
(SEQ ID NO: 1410)
TGGLQN,
(SEQ ID NO: 1411)
TAWVQK,
(SEQ ID NO: 1412)
TGRVQT,
(SEQ ID NO: 1413)
NTQVQN,
(SEQ ID NO: 1414)
TGWLQN,
(SEQ ID NO: 1415)
TYAVQN,
(SEQ ID NO: 1416)
TLSVQN,
(SEQ ID NO: 1417)
NLPVQN,
(SEQ ID NO: 1418)
TSSDQN,
(SEQ ID NO: 1419)
ATSVQN,
(SEQ ID NO: 1420)
TAWFQN,
(SEQ ID NO: 1421)
ATQVQN,
(SEQ ID NO: 1422)
VSSVKN,
(SEQ ID NO: 1252)
LSSVQN,
(SEQ ID NO: 1423)
TGSGQN,
(SEQ ID NO: 1424)
LQPVQT,
(SEQ ID NO: 1225)
VSAVKN,
(SEQ ID NO: 1426)
TQAVQN,
(SEQ ID NO: 1427)
TGWVQK,
(SEQ ID NO: 1428)
VMPVQN,
(SEQ ID NO: 1429)
TVSVQK,
(SEQ ID NO: 1430)
TAWAQN,
(SEQ ID NO: 1431)
NALVQN,
(SEQ ID NO: 1432)
RMLVQN,
(SEQ ID NO: 1433)
TVAVKN,
(SEQ ID NO: 1434)
RQLVQN,
(SEQ ID NO: 1435)
TLGVQN,
(SEQ ID NO: 1436)
LGPVQT,
(SEQ ID NO: 1437)
TGSVKN,
(SEQ ID NO: 1438)
TMSDQN,
(SEQ ID NO: 1439)
LASVKN,
(SEQ ID NO: 1440)
TGFVQN,
(SEQ ID NO: 1441)
SALVQN,
(SEQ ID NO: 1442)
TGWVKT,
(SEQ ID NO: 1442)
SQLVQN,
(SEQ ID NO: 1443)
TGWGQN,
(SEQ ID NO: 1444)
LSAVQN,
(SEQ ID NO: 1445)
TMQVQT,
(SEQ ID NO: 1446)
TGQVQN,
(SEQ ID NO: 1447)
TSPVKN,
(SEQ ID NO: 1448)
LSQVQT,
(SEQ ID NO: 1449)
TGSVLN,
(SEQ ID NO: 1450)
TNGVQN,
(SEQ ID NO: 1451)
TGWVKK,
(SEQ ID NO: 1452)
AAAVQN,
(SEQ ID NO: 1453)
SAVVQN,
(SEQ ID NO: 1454)
PGWVQH,
(SEQ ID NO: 1455)
TASDON,
(SEQ ID NO: 1251)
LSGVQN,
(SEQ ID NO: 1456)
SSRVQN,
(SEQ ID NO: 1457)
SPPVQN,
(SEQ ID NO: 1458)
VQPVQT,
(SEQ ID NO: 1459)
TSSVKN,
(SEQ ID NO: 1460)
LSPLQN,
(SEQ ID NO: 1461)
VSQVQK,
(SEQ ID NO: 1462)
LVGVQN,
(SEQ ID NO: 1463)
TLSVKN,
(SEQ ID NO: 1464)
TGWFQK,
(SEQ ID NO: 1465)
TPAVQN,
(SEQ ID NO: 1466)
TVGVKN,
(SEQ ID NO: 1467)
KGWDQN,
(SEQ ID NO: 1468)
TAWVLN,
(SEQ ID NO: 1469)
VPPVQN,
(SEQ ID NO: 1470)
ATGVQN,
(SEQ ID NO: 1471)
TGGVQI,
(SEQ ID NO: 1472)
TGWVLN,
(SEQ ID NO: 1473)
TAWGQN,
(SEQ ID NO: 1474)
TGWVHN,
(SEQ ID NO: 1475)
LGSVQT,
(SEQ ID NO: 1476)
SANVQN,
(SEQ ID NO: 1477)
TGGVQD,
(SEQ ID NO: 1478)
TMAVKN,
(SEQ ID NO: 1479)
TASVKN,
(SEQ ID NO: 1480)
SSPVQK,
(SEQ ID NO: 1481)
TGTVQT,
(SEQ ID NO: 1482)
TGWVQI,
(SEQ ID NO: 1483)
TVWVKN,
(SEQ ID NO: 1484)
SQQVQN,
(SEQ ID NO: 1485)
VGSVQT,
(SEQ ID NO: 1486)
SSMVQN,
(SEQ ID NO: 1487)
TSPVQK,
(SEQ ID NO: 1488)
AVGVQN,
(SEQ ID NO: 1489)
VAPVQN,
(SEQ ID NO: 1490)
TLPVQK,
(SEQ ID NO: 1491)
TGRVQH,
(SEQ ID NO: 1492)
TPSVQN,
(SEQ ID NO: 1493)
TGWVEN,
(SEQ ID NO: 1494)
RGWVQN,
(SEQ ID NO: 1495)
TGSVEN,
(SEQ ID NO: 1496)
SSLVQN,
(SEQ ID NO: 1497)
TAWVKN,
(SEQ ID NO: 1498)
TYSVQN,
(SEQ ID NO: 1499)
LAAVQT,
(SEQ ID NO: 1500)
TALVKN,
(SEQ ID NO: 1501)
TGWVQY,
(SEQ ID NO: 1502)
TLPVQT,
(SEQ ID NO: 1503)
TGLVQH,
(SEQ ID NO: 1504)
TPTVQN,
(SEQ ID NO: 1505)
TASVQK,
(SEQ ID NO: 1506)
TSPVQI,
(SEQ ID NO: 1507)
IGWVQN,
(SEQ ID NO: 1508)
TGWDKN,
(SEQ ID NO: 1509)
KSSVQN,
(SEQ ID NO: 1510)
TGYVQN,
(SEQ ID NO: 1511)
RGWVQT,
(SEQ ID NO: 1512)
RSLVQN,
(SEQ ID NO: 1513)
TGGVEN,
(SEQ ID NO: 1514)
TGCVRN,
(SEQ ID NO: 1516)
LSPVQS,
(SEQ ID NO: 1517)
TGPVQT,
(SEQ ID NO: 1518)
TVGVQK,
(SEQ ID NO: 1519)
TASGQN,
(SEQ ID NO: 1520)
SVSVQN,
(SEQ ID NO: 1521)
SGPVQT,
(SEQ ID NO: 1522)
VMSVKN,
(SEQ ID NO: 1523)
LGSVQK,
(SEQ ID NO: 1524)
TGLVLN,
(SEQ ID NO: 1525)
TSNVQN,
(SEQ ID NO: 1526)
TGWGHN,
(SEQ ID NO: 1527)
SQMVQN,
(SEQ ID NO: 1528)
TVSVHN,
(SEQ ID NO: 1529)
LSSVQT,
(SEQ ID NO: 1530)
TASVRN,
(SEQ ID NO: 1531)
VPAVQN,
(SEQ ID NO: 1532)
TGRVQK,
(SEQ ID NO: 1533)
AMSVQN,
(SEQ ID NO: 1534)
TAWVHN,
(SEQ ID NO: 1535)
TGLVRN,
(SEQ ID NO: 1536)
RTLVQT,
(SEQ ID NO: 1537)
TGSIQN,
(SEQ ID NO: 1538)
LSSVKN,
(SEQ ID NO: 1539)
TLQVQK,
(SEQ ID NO: 1540)
VGSVKN,
(SEQ ID NO: 1541)
LAPLQN,
(SEQ ID NO: 1542)
TPGVQN,
(SEQ ID NO: 1542)
LSAVQT,
(SEQ ID NO: 1543)
TGVVQN,
(SEQ ID NO: 1544)
VPQVQN,
(SEQ ID NO: 1545)
TGCVOK,
(SEQ ID NO: 1546)
TRWVQT,
(SEQ ID NO: 1547)
TGLDON,
(SEQ ID NO: 1548)
VSSVHN,
(SEQ ID NO: 1549)
KGWVQT,
(SEQ ID NO: 1550)
SLPVQN,
(SEQ ID NO: 1551)
TTSVHN,
(SEQ ID NO: 1552)
TVWVQN,
(SEQ ID NO: 1553)
TAQLQN,
(SEQ ID NO: 1554)
TRWVKN,
(SEQ ID NO: 1555)
TAWIQN,
(SEQ ID NO: 1556)
LSQVKN,
(SEQ ID NO: 1557)
TSTVKN,
(SEQ ID NO: 1558)
ALPVQN,
(SEQ ID NO: 1559)
TSMVQT,
(SEQ ID NO: 1560)
TSSVQH,
(SEQ ID NO: 1561)
TAMVKN,
(SEQ ID NO: 1562)
TPWVQN,
(SEQ ID NO: 1563)
TPRVQN,
(SEQ ID NO: 1564)
SSSVQN,
(SEQ ID NO: 1565)
RPPVQN,
(SEQ ID NO: 1566)
LAGVKN,
(SEQ ID NO: 1567)
TSPAQN,
(SEQ ID NO: 1568)
RSPVQT,
(SEQ ID NO: 1569)
TGWVPH,
(SEQ ID NO: 1570)
PGWGQN,
(SEQ ID NO: 1571)
IPPVQN,
(SEQ ID NO: 1572)
TGRVKN,
(SEQ ID NO: 1573)
TGRLQN,
(SEQ ID NO: 1574)
LSSVQH,
(SEQ ID NO: 1575)
AGWVQT,
(SEQ ID NO: 1576)
TGLVQS,
(SEQ ID NO: 1577)
TGCVQI,
(SEQ ID NO: 1578)
RPGVQN,
(SEQ ID NO: 1579)
TAAVQH,
(SEQ ID NO: 1580)
TGCDQN,
(SEQ ID NO: 1581)
TGRVRN,
(SEQ ID NO: 1582)
TGRDQN;
(ii)
(SEQ ID NO: 1192)
TTKVQN,
(SEQ ID NO: 1158)
TMKVQN,
(SEQ ID NO: 1207)
VAQVQN,
(SEQ ID NO: 3995)
TAWDQN,
(SEQ ID NO: 3698)
TGSVQH,
(SEQ ID NO: 1217)
VKQVQN,
(SEQ ID NO: 3868)
SAPVQN,
(SEQ ID NO: 3929)
LSKVQN,
(SEQ ID NO: 3905)
LAPVQN,
(SEQ ID NO: 3907)
LAQVQN,
(SEQ ID NO: 3985)
TAKVQN,
(SEQ ID NO: 3972)
SAKVQN,
(SEQ ID NO: 3999)
TGCFQN,
(SEQ ID NO: 1171)
TQKVQN,
(SEQ ID NO: 1197)
TVAVQN,
(SEQ ID NO: 3931)
LSPVQN,
(SEQ ID NO: 1195)
TTQVQN,
(SEQ ID NO: 3990)
TAQVQN,
(SEQ ID NO: 3951)
RIAVQN,
(SEQ ID NO: 3948)
RASVQN,
(SEQ ID NO: 1193)
TTPVQN,
(SEQ ID NO: 3867)
LTPVQN,
(SEQ ID NO: 3980)
STPVQN,
(SEQ ID NO: 3875)
TSPVQN,
(SEQ ID NO: 1161)
TMQVQN,
(SEQ ID NO: 1181)
TSKVQN,
(SEQ ID NO: 1237)
VSQVQN,
(SEQ ID NO: 3882)
VSPVQN,
(SEQ ID NO: 1200)
TVQVQN,
(SEQ ID NO: 1242)
VTAVQN,
(SEQ ID NO: 3958)
RQPVQN,
(SEQ ID NO: 3898)
ISGVQN,
(SEQ ID NO: 1232)
VRPVQN,
(SEQ ID NO: 3911)
LGPVQN,
(SEQ ID NO: 1167)
TNQVQN,
(SEQ ID NO: 1229)
VQQVQN,
(SEQ ID NO: 1206)
VANVQN,
(SEQ ID NO: 3884)
AAPVQN,
(SEQ ID NO: 3964)
RSTVQN,
(SEQ ID NO: 1156)
TMAVQN,
(SEQ ID NO: 3896)
IQPVQN,
(SEQ ID NO: 3892)
IASVQN,
(SEQ ID NO: 3881)
TVSVQN,
(SEQ ID NO: 3950)
RGSVQN,
(SEQ ID NO: 3942)
NSPVQN,
(SEQ ID NO: 3925)
LQPVQN,
(SEQ ID NO: 1243)
VTGVQN,
(SEQ ID NO: 1221)
VMQVQN,
(SEQ ID NO: 3976)
SMAVQN,
(SEQ ID NO: 1211)
VGKVQN,
(SEQ ID NO: 3897)
IQSVQN,
(SEQ ID NO: 3888)
CSPVQN,
(SEQ ID NO: 3927)
LQRVQN,
(SEQ ID NO: 3996)
TAWVQH,
(SEQ ID NO: 1169)
TPPVQN,
(SEQ ID NO: 1244)
VTKVQN,
(SEQ ID NO: 3869)
SSPVQN,
(SEQ ID NO: 3885)
AGPVQN,
(SEQ ID NO: 3908)
LARVQN,
(SEQ ID NO: 1196)
TTTVQN,
(SEQ ID NO: 3673)
TGGFQN,
(SEQ ID NO: 1155)
TLQVQN,
(SEQ ID NO: 1163)
TMSVQN,
(SEQ ID NO: 1205)
VAKVQN,
(SEQ ID NO: 3946)
RAAVQN,
(SEQ ID NO: 1199)
TVGVQN,
(SEQ ID NO: 3924)
LNPVQN,
(SEQ ID NO: 3932)
LSQVQN,
(SEQ ID NO: 1151)
TKPVQN,
(SEQ ID NO: 1166)
TNAVQN,
(SEQ ID NO: 3910)
LATVQN,
(SEQ ID NO: 1245)
VTPVQN,
(SEQ ID NO: 1226)
VQAVQN,
(SEQ ID NO: 3874)
TTSVQN,
(SEQ ID NO: 3889)
CTPVQN,
(SEQ ID NO: 3984)
TAGVQN,
(SEQ ID NO: 1185)
TSQVQN,
(SEQ ID NO: 1159)
TMNVQN,
(SEQ ID NO: 1188)
TSTVQN,
(SEQ ID NO: 1216)
VKPVQN,
(SEQ ID NO: 3886)
ASPVQN,
(SEQ ID NO: 1203)
VAAVQN,
(SEQ ID NO: 3915)
LKSVQN,
(SEQ ID NO: 3890)
IAAVQN,
(SEQ ID NO: 3983)
TAAVQN,
(SEQ ID NO: 1150)
TKAVQN,
(SEQ ID NO: 1139)
TGSVQS,
(SEQ ID NO: 1235)
VSNVQN,
(SEQ ID NO: 3988)
TAPVQN,
(SEQ ID NO: 3921)
LMPVQN,
(SEQ ID NO: 3914)
LHPVQN,
(SEQ ID NO: 3947)
RAQVQN,
(SEQ ID NO: 3936)
LTNVQN,
(SEQ ID NO: 3969)
RTTVQN,
(SEQ ID NO: 1189)
TSVVQN,
(SEQ ID NO: 3956)
RMSVQN,
(SEQ ID NO: 1212)
VGNVQN,
(SEQ ID NO: 3922)
LMQVQN,
(SEQ ID NO: 3993)
TATVQN,
(SEQ ID NO: 1214)
VHPVQN,
(SEQ ID NO: 1236)
VSPVQT,
(SEQ ID NO: 3900)
ISSVQN,
(SEQ ID NO: 1208)
VASVQN,
(SEQ ID NO: 1177)
TRWDQN,
(SEQ ID NO: 1165)
TMTVQN,
(SEQ ID NO: 3963)
RSSVQN,
(SEQ ID NO: 3877)
TAWVQN,
(SEQ ID NO: 3966)
RTGVQN,
(SEQ ID NO: 1209)
VATVQN,
(SEQ ID NO: 1246)
VTSVQN,
(SEQ ID NO: 3883)
VSSVQN,
(SEQ ID NO: 1168)
TNSVQN,
(SEQ ID NO: 1215)
VKAVQN,
(SEQ ID NO: 3975)
SGPVQN,
(SEQ ID NO: 3696)
TGPVQN,
(SEQ ID NO: 3986)
TAMVQN,
(SEQ ID NO: 1174)
TQPVQN,
(SEQ ID NO: 1175)
TQQVQN,
(SEQ ID NO: 1238)
VSRVQN,
(SEQ ID NO: 1147)
TGWVQP,
(SEQ ID NO: 1233)
VSAVQN,
(SEQ ID NO: 1219)
VLSVQN,
(SEQ ID NO: 1170)
TQHVQN,
(SEQ ID NO: 3909)
LASVQN,
(SEQ ID NO: 3945)
QAPVQN,
(SEQ ID NO: 3939)
NAQVQN,
(SEQ ID NO: 3887)
ATPVQN,
(SEQ ID NO: 1228)
VQPVQN,
(SEQ ID NO: 1190)
TTAVQN,
(SEQ ID NO: 1149)
TGWVRN,
(SEQ ID NO: 3866)
LAAVQN,
(SEQ ID NO: 1184)
TSPDQN,
(SEQ ID NO: 3960)
RSGVQN,
(SEQ ID NO: 3677)
TGGVQT,
(SEQ ID NO: 1145)
TGWVPN,
(SEQ ID NO: 1194)
TTPVQT,
(SEQ ID NO: 3916)
LMAVQN,
(SEQ ID NO: 1160)
TMPVQN,
(SEQ ID NO: 3904)
LANVQN,
(SEQ ID NO: 1241)
VSTVQN,
(SEQ ID NO: 3974)
SAQVQN,
(SEQ ID NO: 3943)
NTPVQN,
(SEQ ID NO: 1240)
VSSVQT,
(SEQ ID NO: 1201)
TVSVKN,
(SEQ ID NO: 1247)
TGLVQN,
(SEQ ID NO: 3879)
TGSVQN,
(SEQ ID NO: 3672)
TGGAQN,
(SEQ ID NO: 3994)
TAVVQN,
(SEQ ID NO: 3954)
RLGVQN,
(SEQ ID NO: 3967)
RTLVQN,
(SEQ ID NO: 1172)
TQMVQN,
(SEQ ID NO: 3901)
ITPVQN,
(SEQ ID NO: 1202)
TVWVQK,
(SEQ ID NO: 3959)
RSAVQN,
(SEQ ID NO: 3991)
TASVQN,
(SEQ ID NO: 1157)
TMGVQN,
(SEQ ID NO: 3675)
TGGVQH,
(SEQ ID NO: 1230)
VQSVQN,
(SEQ ID NO: 3878)
TGGVQN,
(SEQ ID NO: 3899)
ISPVQN,
(SEQ ID NO: 1248)
TGWVKN,
(SEQ ID NO: 3697)
TGSAQN,
(SEQ ID NO: 3871)
TGWAQN,
(SEQ ID NO: 1164)
TMSVQT,
(SEQ ID NO: 1210)
VGGVQN,
(SEQ ID NO: 3906)
LAPVQT,
(SEQ ID NO: 3876)
TALVQN,
(SEQ ID NO: 3902)
LAGVQN,
(SEQ ID NO: 3965)
RTAVQN,
(SEQ ID NO: 3961)
RSPVQN,
(SEQ ID NO: 3873)
TLAVQN,
(SEQ ID NO: 3903)
LAHVQN,
(SEQ ID NO: 1182)
TSLVQN,
(SEQ ID NO: 3955)
RLSVQN,
(SEQ ID NO: 3978)
LMGVQN,SMQVQN,
(SEQ ID NO: 1176)
TQTVQN,
(SEQ ID NO: 1142)
TGWEQN,
(SEQ ID NO: 1213)
VGSVQN,
(SEQ ID NO: 1234)
VSGVQN,
(SEQ ID NO: 1220)
VMAVQN,
(SEQ ID NO: 3893)
IGGVQN,
(SEQ ID NO: 3891)
IAGVQN,
(SEQ ID NO: 3870)
TGRVQN,
(SEQ ID NO: 3928)
LSHVQN,
(SEQ ID NO: 1231)
VQTVQN,
(SEQ ID NO: 3880)
TGWDQN,
(SEQ ID NO: 3957)
RNSVQN,
(SEQ ID NO: 1154)
TLPVQN,
(SEQ ID NO: 1152)
TKQVQN,
(SEQ ID NO: 3912)
LGQVQN,
(SEQ ID NO: 3941)
NMQVQN,
(SEQ ID NO: 3944)
NVQVQN,
(SEQ ID NO: 3699)
TGSVQI,
(SEQ ID NO: 3949)
RGGVQN,
(SEQ ID NO: 1222)
VMSVQN,
(SEQ ID NO: 1191)
TTGVQN,
(SEQ ID NO: 3930)
LSPVQK,
(SEQ ID NO: 3933)
LSRVQN,
(SEQ ID NO: 1239)
VSSVQK,
(SEQ ID NO: 3989)
TAPVQT,
(SEQ ID NO: 3938)
MAPVQN,
(SEQ ID NO: 3895)
ILGVQN,
(SEQ ID NO: 3992)
TASVQT,
(SEQ ID NO: 3697)
TGSLQN,
(SEQ ID NO: 1141)
TGTVQN,
(SEQ ID NO: 1140)
TGSVQT,
(SEQ ID NO: 1187)
TSSVQT,
(SEQ ID NO: 1180)
TSHVQN,
(SEQ ID NO: 3952)
RIGVQN,
(SEQ ID NO: 1144)
TGWGQT,
(SEQ ID NO: 3973)
SAMVQN,
(SEQ ID NO: 1183)
TSMVQN,
(SEQ ID NO: 3977)
SMGVQN,
(SEQ ID NO: 3979)
SMSVQN,
(SEQ ID NO: 1186)
TSSVQN,
(SEQ ID NO: 1179)
TSGVQN,
(SEQ ID NO: 3998)
TGAVQN,
(SEQ ID NO: 1225)
VNSVQN,
(SEQ ID NO: 1204)
VAGVQN,
(SEQ ID NO: 3894)
IGSVQN,
(SEQ ID NO: 3913)
LGSVQN,
(SEQ ID NO: 1224)
VNGVQN,
(SEQ ID NO: 3935)
LTAVQN,
(SEQ ID NO: 1227)
VQNVQN,
(SEQ ID NO: 1153)
TKSVQN,
(SEQ ID NO: 3982)
SVGVQN,
(SEQ ID NO: 3997)
TAWVQT,
(SEQ ID NO: 3940)
NASVQN,
(SEQ ID NO: 1178)
TSAVQN,
(SEQ ID NO: 1162)
TMSVKN,
(SEQ ID NO: 3987)
TANVQN,
(SEQ ID NO: 1143)
TGWFQN,
(SEQ ID NO: 3674)
TGGVLN,
(SEQ ID NO: 3937)
LTSVQN,
(SEQ ID NO: 1148)
TGWVQT,
(SEQ ID NO: 3962)
RSQVQN,
(SEQ ID NO: 3953)
RIPVQN,
(SEQ ID NO: 1146)
TGWVQD,
(SEQ ID NO: 3970)
RVEVQN,
(SEQ ID NO: 1218)
VLPVQN,
(SEQ ID NO: 3676)
TGGVQK,
(SEQ ID NO: 3981)
SVAVQN,
(SEQ ID NO: 3926)
LQQVQN,
(SEQ ID NO: 3934)
LSTVQN,
(SEQ ID NO: 3971)
SAAVQN,
(SEQ ID NO: 3968)
RTSVQN,
(SEQ ID NO: 1173)
TQNVQN,
(SEQ ID NO: 1223)
VNAVQN,
(SEQ ID NO: 1198)
TVAVQT,
(SEQ ID NO: 3923)
LMSVQN,
TGWVQN;

(iii) amino acid sequence comprising any portion of an amino acid sequence in (i) or (ii), e.g., any 2, 3, 4, or 5 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).
181. The isolated AAV particle of any one of embodiments 170-180, wherein [D][E] is or comprises TGWVQN (SEQ ID NO: 3865), TGWVPN (SEQ ID NO: 1145), or LSPVKN (SEQ ID NO: 1404).
182. The isolated AAV particle of any one of embodiments 170-181, wherein:
   (i) [B] is or comprises: VHLY (SEQ ID NO: 3741), VHHY (SEQ ID NO: 3747), VHIY (SEQ ID NO: 3746), VNLY (SEQ ID NO: 3744), VQLY (SEQ ID NO: 3751), IHLY (SEQ ID NO: 3752), LHLY (SEQ ID NO: 3749), VPLY(SEQ ID NO: 3743), VDLY (SEQ ID NO: 3753), AHLY (SEQ ID NO: 3754), VHRY (SEQ ID NO: 3745), FHLY (SEQ ID NO: 3748), DHLY (SEQ ID NO: 3750), VLLY (SEQ ID NO: 3755), GHLY (SEQ ID NO: 3756), VRLY (SEQ ID NO: 3757), VHVY(SEQ ID NO: 3742), or VYLY (SEQ ID NO: 3758);
   (ii) [C] is or comprises: AQAQ (SEQ ID NO: 3759), AQPQ (SEQ ID NO: 3761), AKAQ (SEQ ID NO: 3763), DQAQ (SEQ ID NO: 3766), SQAQ (SEQ ID NO: 3760), AHAQ (SEQ ID NO: 3764), AQEQ (SEQ ID NO: 3770), AQAK (SEQ ID NO: 3768), ALAQ (SEQ ID NO: 3771), APAQ (SEQ ID NO: 3767), ARAQ SEQ ID NO: 3772), AQAH (SEQ ID NO: 3769), AQAP (SEQ ID NO: 3765), or TQAQ (SEQ ID NO: 3773);
   (iii) [D] is or comprises: TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG; and/or
   (iv) [E] is or comprises: VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH.
183. The isolated AAV particle of any one of embodiments 170-182, wherein:
   (i) [B] is or comprises: VHLY (SEQ ID NO: 3741), VHHY (SEQ ID NO: 3747), or VHIY (SEQ ID NO: 3746);
   (ii) [C] is or comprises: AQAQ (SEQ ID NO: 3759), AQPQ (SEQ ID NO: 3761), AKAQ (SEQ ID NO: 3763), DQAQ (SEQ ID NO: 3766), or SQAQ (SEQ ID NO: 3760);
   (iii) [D] is or comprises: TTK, TMK, VAQ, TAW, TGS, VKQ, SAP, LSK, LAP, LAQ, TAK, SAK, TGC, TQK, TVA, LSP, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, TGW, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, or LMS; and/or
   (iv) [E] is or comprises: VQN, DQN, VQH, FQN, VQS, VQT, VQP, VRN, VPN, VKN, AQN, VQK, EQN, VQI, LQN, GQT, VLN, or VQD.
184. The isolated AAV particle of any one of embodiments 170-183, wherein [A][B][C][D][E] comprises:
   (i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
185. The isolated AAV particle of any one of embodiments 170-184, wherein [A][B][C][D][E] comprises:
   (i) the amino acid sequence of any of SEQ ID NOs: 139, 143, 148, 149, 151, 153-158, 160-163, 166, 168, 170, 171, 173-175, 177-179, 181, 182, 184-188, 191-197, 199, 200, 201-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404, 405, 406-411, 413-415, 417, 419-429, 432, 433, 435-438, 440-442, 444-447, 450-454, 456, 458-461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
186. The isolated AAV particle of any one of embodiments 170-185, wherein [A][B][C][D][E] comprises the amino acid sequence of PLNGAVHLYAQAQTGWVPN (SEQ ID NO: 314).
187. The isolated AAV particle of any one of embodiments 170-185, wherein [A][B][C][D][E] comprises the amino acid sequence of PLNGAVHLYAQAQLSPVKN (SEQ ID NO: 566).
188. The isolated AAV particle of any one of embodiments 170-185, wherein [A][B][C][D][E] comprises the amino acid sequence the amino acid sequence of PLNGAVHLYAQAQTGWVQN (SEQ ID NO: 476).
189. The isolated AAV particle of any one of embodiments 131-188, wherein [A][B] is present in loop VIII.
190. The isolated AAV particle of any one of embodiments 170-189, wherein [C], [D], and/or [E] is present in loop VIII.
191. The isolated AAV particle of any one of embodiments 170-190, wherein [A][B][C][D][E] is present in loop VIII.
192. The isolated AAV particle of any one of embodiments 131-191, which comprises an amino acid other than A at position 587 and/or an amino acid other than Q at position 588, numbered according to SEQ ID NO: 138.
193. The isolated AAV particle of any one of embodiments 131-192, which comprises:
   (i) the amino acid P at position 587, numbered according to SEQ ID NO: 5, 8, 138, or 3636; and/or
   (ii) the amino acid L at position 588, numbered according to SEQ ID NO: 5, 8, 138, or 3636.
194. The isolated AAV particle of any one of embodiments 131-193, wherein [A] is present immediately subsequent to position 586, relative to a reference sequence numbered according to the amino acid sequence of SEQ ID NO: 5, 8, 138 or 3636.
195. The isolated AAV particle of any one of embodiments 131-194, wherein [A] is present immediately subsequent to position 586, and replaces positions 587 and 588 (e.g., A587 and Q588), numbered according to SEQ ID NO: 138.
196. The isolated AAV particle of any one of embodiments 131-195, wherein [A] replaces positions 587 and 588 (e.g., A587 and Q588), numbered according to SEQ ID NO: 138.
197. The isolated AAV particle of any one of embodiments 131-196, wherein [A] corresponds to positions 587-591 of SEQ ID NO: 5, 8, or 3636.
198. The isolated AAV particle of any one of embodiments 131-197, wherein [B] is present immediately subsequent to [A].
199. The isolated AAV particle of any one of embodiments 131-198, wherein [B] is present immediately subsequent to [A], wherein [A] is present immediately subsequent to position 586, relative to a reference sequence numbered according to the amino acid sequence of SEQ ID NO: 5, 8, 138 or 3636.
200. The isolated AAV particle of any one of embodiments 131-199, wherein [B] is present immediately subsequent to [A], wherein [A] is present immediately subsequent to position 586 and replaces positions 587 and 588 (e.g., A587 and Q588), numbered according to SEQ ID NO: 138.
201. The isolated AAV particle of any one of embodiments 131-200, wherein [B] corresponds to positions 592 to 595 of SEQ ID NO: 5, 8, or 3636.
202. The isolated AAV particle of any one of embodiments 131-201, wherein [A][B] replaces positions 587 and 588 (e.g., A587 and Q588), numbered according to SEQ ID NO: 138.
203. The isolated AAV particle of any one of embodiments 131-202, wherein [A][B] is present immediately subsequent to position 586, numbered according to SEQ ID NO: 138.
204. The isolated AAV particle of any one of embodiments 131-203, wherein [A][B] is present immediately subsequent to position 586 and replaces positions 587 and 588 (e.g., A587 and Q588), numbered according to SEQ ID NO: 138.
205 The isolated AAV particle of any one of embodiments 131-204, wherein [A][B] corresponds to positions 587-595 of SEQ ID NO: 5, 8, or 3636.
206. The isolated AAV particle of any one of embodiments 143-204, wherein [C] is present immediately subsequent to position 588, relative to a reference sequence numbered according to the amino acid sequence of SEQ ID NO: 5, 8, 138 or 3636.
207. The isolated AAV particle of any one of embodiments 143-206, wherein [C] replaces positions 589-592 (e.g., A589, Q590, A591, Q592), numbered according to the amino acid sequence of SEQ ID NO: 138.
208. The isolated AAV particle of any one of embodiments 143-207, wherein [C] is present immediately subsequent to position 588, and replaces positions 589-592 (e.g., A589, Q590, A591, Q592), numbered according to the amino acid sequence of SEQ ID NO: 138.
209. The isolated AAV particle of any one of embodiments 143-208, wherein [C] corresponds to positions 596-599 of SEQ ID NO: 5, 8, or 3636.
210. The isolated AAV particle of any one of embodiments 143-209, wherein [A][B][C] is present immediately subsequent to position 586, numbered according to SEQ ID NO: 138.
211. The isolated AAV particle of any one of embodiments 143-210, wherein [A][B][C] replaces positions 587-592 (e.g., A587, Q588, A589, Q590, A591, Q592), numbered according to SEQ ID NO: 138.
212. The isolated AAV particle of any one of embodiments 143-211, wherein [A][B][C] is present immediately subsequent to position 586 and replaces positions 587-592 (e.g., A587, Q588, A589, Q590, A591, Q592), numbered according to SEQ ID NO: 138.
213. The isolated AAV particle of any one of embodiments 143-212, wherein [A][B][C] corresponds to positions 587-599 of SEQ ID NO: 5, 8, or 3636.
214. The isolated AAV particle of any one of embodiments 159-213, wherein [D] is present immediately subsequent to position 592, relative to a reference sequence numbered according to the amino acid sequence of SEQ ID NO: 5, 8, 138 or 3636.
215. The isolated AAV particle of any one of embodiments 159-214, wherein [D] replaces positions 593-595 (e.g., T593, G594, W595), numbered according to the amino acid sequence of SEQ ID NO: 138.
216. The isolated AAV particle of any one of embodiments 159-215, wherein [D] is present immediately subsequent to position 592, and replaces positions 593-595 (e.g., T593, G594, W595), numbered according to the amino acid sequence of SEQ ID NO: 138.
217. The isolated AAV particle of any one of embodiments 159-216, wherein [D] corresponds to positions 600-602 of SEQ ID NO: 5, 8, or 3636.
218. The isolated AAV particle of any one of embodiments 159-217, wherein [C][D] is present immediately subsequent to position 588, numbered according to SEQ ID NO: 138.
219. The isolated AAV particle of any one of embodiments 159-218, wherein [C][D] replaces positions 589-595 (e.g., A589, Q590, A591, Q592, T593, G594, W595), numbered according to the amino acid sequence of SEQ ID NO: 138.
220. The isolated AAV particle of any one of embodiments 159-219, wherein [C][D] is present immediately subsequent to 588, and replaces positions 589-595 (e.g., A589, Q590, A591, Q592, T593, G594, W595), numbered according to the amino acid sequence of SEQ ID NO: 138.
221. The isolated AAV particle of any one of embodiments 159-220, wherein [C][D] corresponds to positions 596-602 of SEQ ID NO: 5, 8, or 3636.
222. The isolated AAV particle of any one of embodiments 159-221, wherein [A][B][C][D] is present immediately subsequent to position 586, numbered according to SEQ ID NO: 5, 8, 138, or 3636.
223. The isolated AAV particle of any one of embodiments 159-222, wherein [A][B][C][D] replaces positions 587-595 (e.g., A587, Q588, A589, Q590, A591, Q592, T593, G594, W595), numbered according to SEQ ID NO: 138.
224. The isolated AAV particle of any one of embodiments 159-223, wherein [A][B][C][D] is present immediately subsequent to position 586 and replaces positions 587-595 (e.g., A587, Q588, A589, Q590, A591, Q592, T593, G594, W595), numbered according to SEQ ID NO: 138.
225. The isolated AAV particle of any one of embodiments 159-224, wherein [A][B][C][D] corresponds to positions 587-602 of SEQ ID NO: 5, 8, or 3636.
226. The isolated AAV particle of any one of embodiments 170-225, wherein [E] is present immediately subsequent to position 595, relative to a reference sequence numbered according to the amino acid sequence of SEQ ID NO: 5, 8, 138 or 3636.
227. The isolated AAV particle of any one of embodiments 170-226, wherein [E] replaces positions 596-598 (e.g., V596, Q597, N598), numbered according to the amino acid sequence of SEQ ID NO: 138.
228. The isolated AAV particle of any one of embodiments 170-227, wherein [E] is present immediately subsequent to position 595, and replaces positions 596-598 (e.g., V596, Q597, N598), numbered according to the amino acid sequence of SEQ ID NO: 138.
229. The isolated AAV particle of any one of embodiments 170-228, wherein [E] corresponds to positions 603 to 605 of SEQ ID NO: 5, 8, or 3636.
230. The isolated AAV particle of any one of embodiments 170-229, wherein [A][B][C][D][E] is present immediately subsequent to position 586, according to the amino acid sequence of SEQ ID NO: 5, 8, 138 or 3636.
231. The isolated AAV particle of any one of embodiments 170-230, wherein [A][B][C][D][E] replaces positions 587-598 (e.g., A587, Q588, A589, Q590, A591, Q592, T593, G594, W595, V596, Q597, N598), numbered according to SEQ ID NO: 138.
232. The isolated AAV particle of any one of embodiments 170-231, wherein [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.
233. The isolated AAV particle of any one of embodiments 170-232, wherein [A][B][C][D][E] corresponds to positions 587-605 of SEQ ID NO: 5, 8, or 3636.
234. The isolated AAV particle of any one of embodiments 131-233, wherein the AAV capsid variant comprises from N-terminus to C-terminus, [A][B].
235. The isolated AAV particle of any one of embodiments 143-234, wherein the AAV capsid variant comprises from N-terminus to C-terminus, [A][B][C].
236. The isolated AAV particle of any one of embodiments 159-235, wherein the AAV capsid variant comprises from N-terminus to C-terminus, [A][B][C][D].
237. The isolated AAV particle of any one of embodiments 170-236, wherein the AAV capsid variant comprises from N-terminus to C-terminus, [A][B][C][D][E].
238. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises PLNGAVHLY (SEQ ID NO: 3648) and optionally wherein the AAV capsid variant further comprises one, two, or all of an amino acid other than T at position 593 (e.g., A, L, R, V, C, I, K, M, N, P, Q, S), an amino acid other than G at position 594 (e.g., M, S, A, Q, V, T, L, P, H, K, N, I, Y, or R), and/or an amino acid other than W at position 595 (e.g., S, P, T, A, G, L, Q, H, N, R, K, V, E, F, M, C, or Y), relative to a reference sequence numbered according to the amino acid sequence of SEQ ID NO: 138.
239. The isolated AAV particle of embodiment 238, wherein:
   (i) the amino acid at position 593, relative to a reference sequence numbered according to the amino acid sequence of SEQ ID NO: 138, is: T, A, L, R, V, C, I, K, M, N, P, Q, or S;
   (ii) the amino acid at position 594, relative to a reference sequence numbered according to the amino acid sequence of SEQ ID NO: 138, is: G, M, S, A, Q, V, T, L, P, H, K, N, I, Y, or R; and/or
   (iii) the amino acid at position 595, relative to a reference sequence numbered according to the amino acid sequence of SEQ ID NO: 138, W, S, P, T, A, G, L, Q, H, N, R, K, V, E, F, M, C, or Y.
240. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises an amino sequence comprising the amino acid sequence of PLNGAVHLY (SEQ ID NO: 3648); and which further comprises one, two, three, or all of:
   (i) the amino acid T, A, L, R, V, C, I, K, M, N, P, Q, or S at position 593 numbered according to SEQ ID NO: 138 or at position 600 numbered according to SEQ ID NO: 5, 8, or 3636;
   (ii) the amino acid G, M, S, A, Q, V, T, L, P, H, K, N, I, Y, or R at position 594 numbered according to SEQ ID NO: 138 or at position 601 numbered according to SEQ ID NO: 5, 8, or 3636; and/or
   (iii) the amino acid W, S, P, T, A, G, L, Q, H, N, R, K, V, E, F, M, C, or Y at position 595 numbered according to SEQ ID NO: 138 or at position 602 numbered according to SEQ ID NO: 5, 8, or 3636;
   optionally, provided that the amino acids at positions 593-595, relative to a reference sequence numbered according to the amino acid sequence of SEQ ID NO: 138, does not comprise the amino acid sequence of TGW.
241. The isolated AAV particle of any one of embodiments 238-240, wherein the AAV capsid variant comprises:
   (i) the amino acid L at position 593 numbered according to SEQ ID NO: 138 or at position 600 numbered according to SEQ ID NO: 5, 8, or 3636;
   (ii) the amino acid S at position 594 numbered according to SEQ ID NO: 138 or at position 601 numbered according to SEQ ID NO: 5, 8, or 3636; and/or
   (iii) the amino acid P at position 595 numbered according to SEQ ID NO: 138 or at position 602 numbered according to SEQ ID NO: 5, 8, or 3636.
242. The isolated AAV particle of any one of embodiments 238-241, wherein the AAV capsid variant comprises the amino acid sequence of TMS, ASP, LGS, LSS, RST, TAA, TAG, TAL, TAS, TGT, TMA, TQP, TSA, TSP, TST, TVA, TVS, VMS, VSP, VSS, VTP, TGP, VAS, AAA, AAP, AGP, AGW, ALP, AMS, AQP, ATG, ATP, ATQ, ATS, AVG, CSP, CTP, IAA, IAG, IAS, IGG, IGS, IGW, ILG, IPP, IQP, IQS, ISG, ISP, ISS, ITP, KGW, KSS, LAA, LAG, LAH, LAN, LAP, LAQ, LAR, LAS, LAT, LGP, LGQ, LGT, LHP, LKS, LLP, LMA, LMG, LMP, LMQ, LMS, LNP, LPP, LQP, LQQ, LQR, LSA, LSG, LSH, LSK, LSN, LSP, LSQ, LSR, LST, LTA, LTN, LTP, LTS, LVG, MAP, NAL, NAQ, NAS, NLP, NMQ, NQP, NSP, NTP, NTQ, NTT, NVQ, PGW, QAP, QSP, RAA, RAG, RAQ, RAS, RGG, RGS, RGW, RIA, RIG, RIP, RLG, RLP, RLS, RLT, RML, RMS, RNS, RPG, RPP, RQG, RQL, RQP, RQQ, RQS, RSA, RSG, RSP, RSQ, RSS, RSV, RTA, RTG, RTL, RTP, RTQ, RTS, RTT, RVE, RYS, SAA, SAF, SAG, SAK, SAL, SAM, SAN, SAP, SAQ, SAV, SGP, SLP, SMA, SMG, SMQ, SMS, SPP, SQL, SQM, SQP, SQQ, SSL, SSM, SSP, SSR, SSS, STP, STQ, STS, SVA, SVG, SVQ, SVS, TAK, TAM, TAN, TAP, TAQ, TAT, TAV, TAW, TGA, TGC, TGF, TGG, TGL, TGM, TGQ, TGR, TGS, TGV, TGY, TKA, TKP, TKQ, TKS, TLA, TLG, TLP, TLQ, TLR, TLS, TMG, TMK, TMM, TMN, TMP, TMQ, TMT, TNA, TNG, TNQ, TNS, TPA, TPG, TPP, TPQ, TPR, TPS, TPT, TPW, TQA, TQH, TQK, TQL, TQM, TQN, TQQ, TQR, TQT, TQW, TRW, TSG, TSH, TSK, TSL, TSM, TSN, TSQ, TSS, TSV, TSW, TTA, TTG, TTK, TTP, TTQ, TTR, TTS, TTT, TVG, TVQ, TVW, TYA, TYS, VAA, VAG, VAK, VAN, VAQ, VAT, VGG, VGK, VGN, VGS, VHP, VKA, VKP, VKQ, VLG, VLP, VLS, VMA, VMP, VMQ, VNA, VNG, VNS, VPA, VPP, VPQ, VQA, VQG, VQN, VQP, VQQ, VQS, VQT, VRP, VSA, VSG, VSN, VSQ, VSR, VST, VTA, VTG, VTK, VTS, or VAP at positions 593-595 numbered according to SEQ ID NO: 138 or at positions 600-602 numbered according to SEQ ID NO: 5, 8, or 3636.
243. The isolated AAV particle of any one of embodiments 230-242, wherein the AAV capsid variant comprises the amino acid sequence LSP at positions 593-595 numbered according to SEQ ID NO: 138 or at positions 600-602 numbered according to SEQ ID NO: 5, 8, or 3636.
244. The isolated AAV particle of any one of embodiments 238-243, wherein the AAV capsid variant further comprises one, two, three, or all of an amino acid other than A at position 589 (e.g., D, S, or T), an amino acid other than Q at position 590 (e.g., K, H, L, P, or R), an amino acid other than A at position 591 (e.g., P or E), and/or an amino acid other than Q at position 592 (e.g., H, K, or P).
245. The isolated AAV particle of embodiment 244, wherein:
   (i) the amino acid A, D, S, or T at position 589, numbered according to SEQ ID NO: 138;
   (ii) the amino acid Q, K, H, L, P, or R at position 590, numbered according to SEQ ID NO: 138;
   (iii) the amino acid A, E, or P at position 591, numbered according to SEQ ID NO: 138; and/or
   (iv) the amino acid Q, H, K, or P at position 592, numbered according to SEQ ID NO: 138.
246. The isolated AAV particle of embodiment 244 or 245, wherein the AAV capsid variant comprises the amino acid sequence of:
   (i) AHAQ (SEQ ID NO: 3764), AKAQ (SEQ ID NO: 3763), ALAQ (SEQ ID NO: 3771), APAQ (SEQ ID NO: 3767), AQAH (SEQ ID NO: 3769), AQAK (SEQ ID NO: 3768), AQAP (SEQ ID NO: 3765), AQAQ (SEQ ID NO: 3759), AQEQ (SEQ ID NO: 3770), AQPQ (SEQ ID NO: 3761), ARAQ SEQ ID NO: 3772), DQAQ (SEQ ID NO: 3766), SQAQ (SEQ ID NO: 3760), or TQAQ (SEQ ID NO: 3773) at positions 589-592 numbered according to SEQ ID NO: 138 or at positions 596-599 numbered according to SEQ ID NO: 5, 8, or 3636; or
   (ii) AKAQ (SEQ ID NO: 3763) AQAQ (SEQ ID NO: 3759), AQPQ (SEQ ID NO: 3761), DQAQ (SEQ ID NO: 3766), or SQAQ (SEQ ID NO: 3760) at positions 589-592 numbered according to SEQ ID NO: 138 or at positions 596-599 numbered according to SEQ ID NO: 5, 8, or 3636.
247. The isolated AAV particle of any one of embodiments 238-246, wherein the AAV capsid variant further comprises one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), relative to a reference sequence numbered according to the amino acid sequence of SEQ ID NO: 138.
248. The isolated AAV particle of embodiment 247, wherein:
   (i) the amino acid V, G, F, D, L, A, I, or E at position 596 numbered according to SEQ ID NO: 138 or at position 603 numbered according to SEQ ID NO: 5, 8, or 3636;
   (ii) the amino acid Q, K, R, H, E, L, or P at position 597, numbered according to SEQ ID NO: 138 or at position 604 numbered according to SEQ ID NO: 5, 8, or 3636; and/or
   (iii) the amino acid N, H, K, T, I, S, D, P, or Y at position 598 numbered according to SEQ ID NO: 138 or at position 605 numbered according to SEQ ID NO: 5, 8, or 3636.
249. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises PLNGAVHLY (SEQ ID NO: 3648), and optionally wherein the AAV capsid variant further comprises one, two, or all of an amino acid other than V at position 596 (e.g., G, F, D, L, A, I, or E), an amino acid other than Q at position 597 (e.g., K, R, H, E, L, or P), and/or an amino acid other than N at position 598 (e.g., H, K, T, I, S, D, P, or Y), numbered according to SEQ ID NO: 138.
250. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises PLNGAVHLY (SEQ ID NO: 3648), and further comprising one, two, or all of:
   (i) V, G, F, D, L, A, I, or E at position 596 numbered according to SEQ ID NO: 138 or at position 603 numbered according to SEQ ID NO: 5, 8, or 3636;
   (ii) Q, K, R, H, E, L, or P at position 597 numbered according to SEQ ID NO: 138 or at position 604 numbered according to SEQ ID NO: 5, 8, or 3636; and/or
   (iii) N, H, K, T, I, S, D, P, or Y at position 598 numbered according to SEQ ID NO: 138 or at position 605 numbered according to SEQ ID NO: 5, 8, or 3636;
optionally, provided that the amino acids at positions 596-598 numbered according to the amino acid sequence of SEQ ID NO: 138 or positions 603-605 of SEQ ID NO: 5, 8, or 3636, does not comprise the amino acid sequence of VQN.
251. The isolated AAV particle of any one of embodiments 238-250, which comprises the amino acid P at position 597 numbered according to SEQ ID NO: 138 or at position 604 numbered according to SEQ ID NO: 5, 8, or 3636.
252. The isolated AAV particle of any one of embodiments 238-251, which comprises the amino acid K at position 597 numbered according to SEQ ID NO: 138 or at position 604 numbered according to SEQ ID NO: 5, 8, or 3636.
253. The isolated AAV particle of any one of embodiments 247-252, wherein AAV capsid variant comprises the amino acid sequence of:
   (i) GQN, VQH, VQK, VQT, VQN, FQN, VKN, VQI, DQN, LQN, VQS, VRN, AQN, IQN, VHN, VLN, VEN, VQD, DKN, EQN, FQK, GHN, GQT, VKK, VKT, VPH, VPN, VQP, or VQY at positions 596-598 numbered according to SEQ ID NO: 138 or positions 603-605 numbered according to SEQ ID NO: 5, 8, or 3636; or
   (ii) VQN, VQT, VQK, DQN, VQH, FQN, AQN, VLN, LQN, VQI, VQS, EQN, GQT, VPN, VQD, VQP, VRN, or VKN at positions 596-598 numbered according to SEQ ID NO: 138 or positions 603-605 numbered according to SEQ ID NO: 5, 8, or 3636.
254. The isolated AAV particle of any one of embodiments 247-253, which comprises the amino acid sequence of VKN, VPN, or VQN at positions 596-598 numbered according to SEQ ID NO: 138 or positions 603-605 numbered according to SEQ ID NO: 5, 8, or 3636.
255. The isolated AAV particle of any one of embodiments 247-253, which comprises the amino acid sequence of VEN or VHN at positions 596-598 numbered according to SEQ ID NO: 138 or positions 603-605 numbered according to SEQ ID NO: 5, 8, or 363.
256. The isolated AAV particle of any one of embodiments 238-255, wherein the AAV capsid variant comprises:
   (i) the amino acid sequence of any of SEQ ID NOs: 143, 148, 149, 151, 153-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284, 285, 286, 288-293, 295, 296, 298, 300-314, 316-318, 320-327, 329, 331, 332, 334, 336-344, 346-350, 352-354, 356-367, 369, 371-380, 382-385, 387, 392-394, 396, 397, 399-401, 404-411, 413-415, 417, 419-429, 432, 433, 435-437, 438, 440-442, 444-447, 450-453, 456, 458-461, 464, 465, 467-469, 471-478, 480-483, 487-495, 497, 498, 500-503, 505, 507-512, 515-517, 522-525, 528-532, 534-539, 542-545, 547, 551-554, 558-561, 563-568, 570, 573, 574, 576, 579, 581, 582, 584, 586, 587, 592-596, 598, 601, 604-607, 610, 612, 614-619, 624-629, 631, 633-636, 640, 641, 645, 646, 649, 650, 658, 663, 664, 666, 668, 669, 672, 673, 675, 679, 683, 684, 686, 688, 689, 691, 693, 695, 697, 699, 700, 704, 705, 709-712, 720, 722, 726-731, 733, 736, 740, 745, 749, 750-752, 754, 755, 757, 758, 760-765, 767, 768, 771, 778, 780, 783-787, 792, 794, 797, 799-802, 804, 817, 818, 821, 824, 828, 831, 832, 834-837, 840-845, 847, 848, 851-853, 855, 858, 861, 862, 865, 869, 870-872, 874, 876, 882, 883, 887, 889, 890, 892-895, 897, 901, 903, 904, 905, 907, 910, 911, 913, 915, 919, 920, 923, 924, 926, 927, 929, 931-933, 935, 937, 940, 941, 943, 945-949, 953, 955, 957, 958, 960, 962, 964, 965, 971, 973, 974, 977, 986, 988, 989, 992, 994, 997, 998, 1000, 1004, 1007, 1013, 1015, 1017, 1018, 1020, 1025, 1027, 1029, 1030, 1031, 1033-1035, 1037-1039, 1043, 1046, 1049, 1052, 1056, 1057, 1059, 1062, 1065, 1067, 1068, 1070, 1073, 1075, 1077-1079, 1083-1087, 1089, 1090, 1094, 1100, 1101, 1103, 1106, 1107, 1110, 1111, 1112, 1114, 1115, 1117, 1119, 1125, 1126, 1129, 1132, or 1133;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
257. The isolated AAV particle of any one of embodiments 238-256, wherein the AAV capsid variant comprises:
   (i) the amino acid sequence of any of SEQ ID NOs: 143, 148-151, 153-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-318, 320-327, 329, 331, 332, 334, 336, 337-339, 340-344, 346-350, 352-354, 356-365, 367, 369, 371-380, 382-385, 387, 392-394, 396, 397, 399-401, 404-411, 413-415, 417, 419, 420-429, 432, 433, 435-438, 440-442, 444-447, 450-453, 456, 458, 459, 460, 461, 464, 465, 467-469, or 471-476;
   (ii) an amino acid sequence comprising any portion of an amino acid sequence in (i), e.g., any 2, 3, 4, 5, 6, 7, 8, 9, 10, 11, 12, 13, 14, 15, 16, 17, or 18 amino acids, e.g., consecutive amino acids, thereof;
   (iii) an amino acid sequence comprising at least one, two, or three but no more than four modifications, e.g., substitutions (e.g., conservative substitutions), insertions, or deletions, relative to any of the amino acid sequences in (i); or
   (iv) an amino acid sequence comprising at least one, two, or three but no more than four different amino acids, relative to any one of the amino acid sequences in (i).
258. The isolated AAV particle of any one of embodiments 238-257, wherein the amino acid sequence of PLNGAVHLY (SEQ ID NO: 3648) is present in loop VIII.
259. The isolated AAV particle of any one of embodiments 238-258, wherein the amino acid sequence of PLNGAVHLY (SEQ ID NO: 3648) is present immediately subsequent to position 586, relative to a reference sequence numbered according to the amino acid sequence of SEQ ID NO: 138.
260. The isolated AAV particle of any one of embodiments 238-259, wherein the amino acid sequence of PLNGAVHLY (SEQ ID NO: 3648) replaces positions 587 and 588 (e.g., A587 and Q588), numbered according to SEQ ID NO: 138.
261. The isolated AAV particle of any one of embodiments 238-260, wherein the amino acid sequence of PLNGAVHLY (SEQ ID NO: 3648) is present immediately subsequent to position 586 and replaces positions 587 and 588 (e.g., A587 and Q588), numbered according to SEQ ID NO: 138.
262. The isolated AAV particle of any one of embodiments 138-261, wherein the amino acid sequence of PLNGAVHLY (SEQ ID NO: 3648) corresponds to positions 587-595 of SEQ ID NO: 5, 8, or 3636.
263. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises X1-X2-X3-X4-X5-X6-X7-X8-X9-X10-X11-X12-X13-X14-X15-X16-X17-X18-X19, wherein:
   (i) X1 is: P, A, D, E, F, G, H, K, L, N, Q, R, S, T, or V;
   (ii) X2 is: L, D, E, F, H, I, M, N, P, Q, R, S, or V;
   (iii) X3 is: N, A, D, E, G, H, I, K, Q, S, T, V, or Y;
   (iv) X4 is: G, A, C, D, E, P, Q, R, S, T, V, or W;
   (v) X5 is: A, C, D, E, F, G, H, I, K, N, P, Q, R, S, T, V, W, or Y;
   (vi) X6 is: V, A, C, D, E, F, G, H, I, K, L, M, N, Q, R, S, T, or Y;
   (vii) X7 is: H, A, D, E, G, I, K, L, M, N, P, Q, R, S, T, V, or Y;
   (viii) X8 is: L, A, D, E, F, G, H, I, K, M, N, P, Q, R, S, T, V, or Y;
   (ix) X9 is: Y, A, C, D, E, F, G, H, I, K, L, M, N, Q, R, S, T, V, or W;
   (x) X10 is: A, C, D, E, F, G, H, I, K, L, N, P, Q, R, S, T, V, or; Y;
   (xi) X11 is: Q, A, D, E, H, K, L, P, R, or T;
   (xii) X12 is: A, D, E, G, H, L, N, P, Q, R, S, T, or V;
   (xiii) X13 is: Q, E, H, K, L, P, R, or T;
   (xiv) X14 is: T, A, C, D, E, F, G, H, I, K, L, M, N, P, Q, R, S, V, W, or Y;
   (xv) X15 is: G, A, C, D, E, F, H, I, K, L, M, N, P, Q, R, S, T, V, W, or Y;
   (xvi) X16 is: W, A, C, D, E, F, G, H, I, K, L, M, N, P, Q, R, S, T, V, or Y;
   (xvii) X17 is: V, A, D, E, F, G, H, I, or L;
   (xviii) X18 is: Q, E, H, K, L, P, or R; and/or
   (xix) X19 is: N, D, H, I, K, P, S, T, or Y.
264. The isolated AAV particle of embodiment 263, wherein:
   (i) X1 is: P, Q, A, S, T, R, H, L, or K;
   (ii) X2 is: L, I, V, H, or R;
   (iii) X3 is: N, D, K, Y, or I;
   (iv) X4 is: G, S, R, C, or A;
   (v) X5 is: A, S, G, N, T, D, Y, Q, V, or C;
   (vi) X6 is: V, I, L, A, F, D, or G;
   (vii) X7 is: H, N, Q, P, D, L, R, or Y;
   (viii) X8 is: L, H, V, I, or R;
   (ix) X9 is Y;
   (x) X10 is: A, D, S, or T;
   (xi) X11 is: Q, K, H, L, P, or R;
   (xii) X12 is: A, P, E, or S;
   (xiii) X13 is: Q, K, H, or P;
   (xiv) X14 is: L, T, V, S, R, I, A, N, C, P, Q, M, or K;
   (xv) X15 is: S, G, M, T, A, K, Q, V, I, R, N, P, L, H, Y;
   (xvi) X16 is: P, W, S, K, Q, G, C, R, A, N, T, V, M, H, L, E, F, or Y;
   (xvii) X17 is: V, D, F, A, E, L, G, or I;
   (xviii) X18 is: Q, R, P, K, L, H, or E; and/or
   (xix) X19 is: N, H, D, S, T, P, K, I, or Y.
265. The isolated AAV particle of embodiment 263 or 264, wherein:
   (i) X1 is: P, A, S, Q, or T;
   (ii) X2 is L or I;
   (iii) X3 is N or D;
   (iv) X4 is G or S;
   (v) X5 is: A, S, G, N, or T;
   (vi) X6 is V;
   (vii) X7 is H;
   (viii) X8 is: L, H, V, or I
   (ix) X9 is Y;
   (x) X10 is: A, D, or S;
   (xi) X11 is Q or K;
   (xii) X12 is A or P;
   (xiii) X13 is Q;
   (xiv) X14 is: L, T, V, S, R, I, A, N, C, P, Q, or M;
   (xv) X15 is: S, G, M, T, A, K, Q, V, I, R, N, P, L, or H;
   (xvi) X16 is: P, W, S, K, Q, G, C, R, A, N, T, V, M, H, L, or E;
   (xvii) X17 is: V, D, F, A, E, L, or G;
   (xviii) X18 is: Q, R, P, K, or L; and/or
   (xix) X19 is: N, H, D, S, T, P, K, or I.
266. The isolated AAV particle of any one of embodiments 263-265, wherein X1-X2-X3-X4-X5-X6-X7-X8-X9-X10-X11-X12-X13-X14-X15-X16-X17-X18-X19 is present immediately subsequent to position 586, numbered according to SEQ ID NO: 5, 8, 138, or 3636.
267. The isolated AAV particle of any one of embodiments 263-266, wherein X1-X2-X3-X4-X5-X6-X7-X8-X9-X10-X11-X12-X13-X14-X15-X16-X17-X18-X19 replaces positions 587-598 (e.g., A587, Q588, A589, Q590, A591, Q592, T593, G594, W595, V596, Q597, N598), numbered according to SEQ ID NO: 138.
268. The isolated AAV particle of any one of embodiments 263-267, wherein X1-X2-X3-X4-X5-X6-X7-X8-X9-X10-X11-X12-X13-X14-X15-X16-X17-X18-X19 is present immediately subsequent to position 586 and replaces positions 587-598 (e.g., A587, Q588, A589, Q590, A591, Q592, T593, G594, W595, V596, Q597, N598), numbered according to SEQ ID NO: 138.
269. The isolated AAV particle of any one of embodiments 263-268, wherein X1-X2-X3-X4-X5-X6-X7-X8-X9-X10-X11-X12-X13-X14-X15-X16-X17-X18-X19 corresponds to positions 587-605 of SEQ ID NO: 5, 8, or 3636.
270. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (a) the amino acid sequence of any one of SEQ ID NOs: 139-1138;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-1138; or
   (c) an amino acid sequence comprising at least one, two, or three but no more than four different amino acids, relative to the amino acid sequence of any one of SEQ ID NOs: 139-1138;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-1138;
   optionally wherein the AAV capsid variant does not comprise the amino acid sequence of TGW at positions 593-595, relative to a reference sequence numbered according to the amino acid sequence of SEQ ID NO: 138.
271. An isolated, e.g., recombinant, AAV particle comprising an AAV capsid variant and a nucleic acid comprising a transgene encoding a SOD1 targeting polynucleotide (e.g., a SOD1 targeting RNA agent, e.g., a siRNA duplex), wherein the AAV capsid variant comprises:
   (a) the amino acid sequence of any one of SEQ ID NOs: 139-476;
   (b) an amino acid sequence comprising at least 3, 4, 5, 6, 7, 8, 9, 10, 11, 12, 13, 14, 15, 16, 17, or 18 consecutive amino acids from any one of SEQ ID NOs: 139-476; or
   (c) an amino acid sequence comprising at least one, two, or three but no more than four different amino acids, relative to the amino acid sequence of any one of SEQ ID NOs: 139-476;
   (d) an amino sequence comprising at least one, two, or three but no more than four modifications, e.g., substitutions (e.g., conservative substitutions), insertions, or deletions, relative to the amino acid sequence of any one of SEQ ID NOs: 139-476;
   optionally wherein the AAV capsid variant does not comprise the amino acid sequence of TGW at positions 593-595, relative to a reference sequence numbered according to the amino acid sequence of SEQ ID NO: 138.
272. An isolated, e.g., recombinant, AAV particle comprising an AAV capsid variant and a nucleic acid comprising a transgene encoding a SOD1 targeting polynucleotide (e.g., a SOD1 targeting RNAi), or a fragment thereof, wherein the AAV capsid variant comprises:
   (a) the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113;
   (b) an amino acid sequence comprising at least 3, 4, 5, 6, 7, 8, 9, 10, 11, 12, 13, 14, 15, 16, 17, or 18 consecutive amino acids from any one of the amino acid sequences in (i); or
   (c) an amino acid sequence comprising at least one, two, or three but no more than four different amino acids, relative to the amino acid sequence of any one of the amino acid sequences in (i);
   (d) an amino sequence comprising at least one, two, or three but no more than four modifications, e.g., substitutions (e.g., conservative substitutions), insertions, or deletions, relative to the amino acid sequence of any one of the amino acid sequences in (i);
   optionally wherein the AAV capsid variant does not comprise the amino acid sequence of TGW at positions 593-595, relative to a reference sequence numbered according to the amino acid sequence of SEQ ID NO: 138.
273. The isolated AAV particle of embodiment 270, wherein the AAV capsid variant comprises an amino acid sequence comprising at least 4, 5, 6, 7, 8, 9, 10, 11, 12, 13, 14, 15, 16, 17, or 18 consecutive amino acids from any one of SEQ ID NOs: 139-1138.
274. The AAV capsid variant of any one of embodiments 270, 271 or 273, wherein the AAV capsid variant comprises an amino acid sequence comprising at least 4, 5, 6, 7, 8, 9, 10, 11, 12, 13, 14, 15, 16, 17, or 18 consecutive amino acids from SEQ ID NO: 314.
275. The AAV capsid variant of any one of embodiments 270 or 273, wherein the AAV capsid variant comprises an amino acid sequence comprising at least 4, 5, 6, 7, 8, 9, 10, 11, 12, 13, 14, 15, 16, 17, or 18 consecutive amino acids from SEQ ID NO: 566.
276. The isolated AAV particle of embodiment 270 or 271, comprising an amino acid sequence comprising at least 4, 5, 6, 7, 8, 9, 10, 11, 12, 13, 14, 15, 16, 17, or 18 consecutive amino acids from any one of SEQ ID NOs: 139-476.
277. The isolated AAV particle of embodiment 270 or 272, wherein the AAV capsid variant comprises an amino acid sequence comprising at least 4, 5, 6, 7, 8, 9, 10, 11, 12, 13, 14, 15, 16, 17, or 18 consecutive amino acids from any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, 1113.
278. The isolated AAV particle of embodiment 270, wherein the AAV capsid variant comprises an amino acid sequence comprising at least one, two, or three but no more than four different amino acids relative to the amino acid sequence of any one of SEQ ID NOs: 139-1138.
279. The isolated AAV particle of embodiment 270 or 271, wherein the AAV capsid variant comprises an amino acid sequence comprising at least one, two, or three but no more than four different amino acids relative to the amino acid sequence of any one of SEQ ID NOs: 139-476.
280. The isolated AAV particle of embodiment 270 or 272, wherein the AAV capsid variant comprises an amino acid sequence comprising at least one, two, or three but no more than four different amino acids relative to the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
281. The isolated AAV particle of embodiment 270, wherein the AAV capsid variant comprises an amino acid sequence comprising at least one, two, or three but no more than four modifications, e.g., substitutions (e.g., conservative substitutions), insertions, or deletions, relative to the amino acid sequence of SEQ ID NOs: 139-1138.
282. The isolated AAV particle of embodiment 270 or 271, wherein the AAV capsid variant comprises an amino acid sequence comprising at least one, two, or three but no more than four modifications, e.g., substitutions (e.g., conservative substitutions), insertions, or deletions, relative to the amino acid sequence of SEQ ID NOs: 139-476.
283. The AAV capsid variant of any one of embodiments 270, 271, 273, 274 or 282, wherein the AAV capsid variant comprises:
   (i) an amino acid sequence comprising at least one, two, or three but no more than four different amino acids relative to the amino acid sequence of SEQ ID NO: 314; or
   (ii) an amino acid sequence comprising at least one, two, or three but no more than four modifications, e.g., substitutions (e.g., conservative substitutions), insertions, or deletions, relative to SEQ ID NO: 314.
284. The AAV capsid variant of any one of embodiments 270, 275 or 282, wherein the AAV capsid variant comprises:
   (i) an amino acid sequence comprising at least one, two, or three but no more than four different amino acids relative to the amino acid sequence of SEQ ID NO: 566; or
   (ii) an amino acid sequence comprising at least one, two, or three but no more than four modifications, e.g., substitutions (e.g., conservative substitutions), insertions, or deletions, relative to SEQ ID NO: 566.
285. The isolated AAV particle of embodiment 270 or 272, wherein the AAV capsid variant comprises an amino acid sequence comprising at least one, two, or three but no more than four modifications, e.g., substitutions (e.g., conservative substitutions), insertions, or deletions, relative to any of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
286. The isolated AAV particle of any one of embodiments 270-285, wherein 4, 5, 6, 7, 8, or 9 consecutive amino acids is not PLNG (SEQ ID NO: 3678), PLNGA (SEQ ID NO: 3679), PLNGAV (SEQ ID NO: 3680), PLNGAVHL (SEQ ID NO: 3682), and/or PLNGAVHLY (SEQ ID NO: 3648).
287. The isolated AAV particle of any one of embodiments 270, 273, 278, 281, wherein the AAV capsid variant comprises the amino acid sequence of any one of SEQ ID NOs: 139-1138.
288. The isolated AAV particle of any one of embodiments 270, 273, 275, 278 or 281, wherein the AAV capsid variant comprises the amino acid sequence of SEQ ID NO: 314.
289. The isolated AAV particle of any one of embodiments 270, 273, 274, 278 or 281, wherein the AAV capsid variant comprises the amino acid sequence of SEQ ID NO: 566.
290. The isolated AAV particle of any one of embodiments 270, 271, 273, 276, 278, 281 or 282, wherein the AAV capsid variant comprises the amino acid sequence of any one of SEQ ID NOs: 139-476.
291. The isolated AAV particle of embodiment 270, 272, 273, 277, 278, 280, 281 or 285, wherein the AAV capsid variant comprises the amino acid sequence of any one of SEQ ID NOs: 140, 142-144, 148-150, 154-158, 160, 161, 163, 165, 166, 168, 170, 171, 173-175, 177-179, 181, 182, 184-197, 199-214, 218-222, 224, 225, 227-241, 243-253, 255-262, 265, 267, 268, 270, 271, 273, 274, 276, 277, 279, 282, 284-286, 288-296, 300-310, 312, 315, 317, 318, 320-323, 326, 327, 331, 332, 334, 336, 337, 339, 340, 341, 343, 344, 346, 349, 351, 352, 356-363, 365-367, 369, 370, 372-376, 378-381, 383-389, 392, 393, 395, 397-400, 404, 407, 408, 411, 412, 415, 417, 420-430, 432, 433, 435-438, 441, 442, 446-448, 451-453, 456, 458, 460, 461, 465, 467-469, 471-473, 475, 476, 478, 480, 482, 485, 488, 490, 492, 493, 495, 498, 500-503, 505, 507, 509, 510, 517, 522-526, 528, 535-538, 540, 543-545, 547, 551, 552, 557, 559, 561, 564, 568, 570, 572-574, 577, 585-588, 592-594, 596, 601, 602, 605, 612, 616, 619, 622, 624, 627, 628, 635, 640, 641, 646, 658, 660, 665, 666, 675, 678, 680, 683, 684, 689, 693, 695, 707, 711, 718, 719, 724, 727, 735, 740, 748, 751, 755, 758, 759, 765, 766, 768, 778, 783, 787, 791, 797, 801, 804, 817, 821, 832, 841, 852, 856, 861, 862, 864, 894, 906, 911, 913, 924, 929, 945, 959, 961, 970, 975, 980, 983, 988, 992, 1009, 1015, 1019, 1027, 1032, 1036, 1038, 1047, 1051, 1061, 1077, 1081, 1095, or 1113.
292. The isolated AAV particle of any one of embodiments 1-291, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises: (a) a VP1 protein comprising the amino acid sequence of SEQ ID NO: 138 or SEQ ID NO: 5, 8, or 3636; (b) a VP2 protein comprising the amino acid sequence of positions 138-736 of SEQ ID NO: 138 or positions 138-743 of SEQ ID NO: 5, 8, or 3636; (c) a VP3 protein comprising the amino acid sequence of positions 203-736 of SEQ ID NO: 138 or positions 203-743 of SEQ ID NO: 5, 8, or 3636; or (d) an amino acid sequence with at least 90% (e.g., at least about 95, 96, 97, 98, or 99%) sequence identity to any of the amino acid sequences in (a)-(c).
293. The isolated AAV particle of any one of embodiments 1-292, wherein the AAV capsid variant comprises an amino acid sequence comprising at least 5, 6, 7, 8, or 9 consecutive amino acids from the amino acid sequence of PLNGAVHLY (SEQ ID NO: 3648), wherein:
   (i) the 5 consecutive amino acids comprise PLNGA (SEQ ID NO: 3679);
   (ii) the 6 consecutive amino acids comprise PLNGAV (SEQ ID NO: 3680);
   (iii) the 7 consecutive amino acids comprise PLNGAVH (SEQ ID NO: 3681);
   (iv) the 8 consecutive amino acids comprise PLNGAVHL (SEQ ID NO: 3682); or
   (v) the 9 consecutive amino acids comprise PLNGAVHLY (SEQ ID NO: 3648),
   wherein the AAV capsid variant comprises the amino acid sequence of any one of SEQ ID NOs: 5, 8, or 3636 or an amino acid sequence with at least 90% (e.g., at least about 95, 96, 97, 98, or 99%) sequence identity to any one of SEQ ID NOs: 5, 8, or 3636.
294. The isolated AAV particle of any one of embodiments 1-293, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
295. The isolated AAV particle of any one of embodiments 1-294, wherein the AAV capsid variant comprises one or two, but no more than three different amino acids (e.g., substitutions, e.g., conservative substitutions) relative to the amino acid sequence of PLNGAVHLY (SEQ ID NO: 3648), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
296. The isolated AAV particle of any one of embodiments 292-295, wherein the amino acid sequence is present immediately subsequent to position 586, numbered according to any one of SEQ ID NO: 5, 8, 138, or 3636, optionally wherein the amino acid replaces positions 587 and 588 (e.g., A587 and Q588), numbered according to SEQ ID NO: 138.
297. The isolated AAV particle of any one of embodiments 292-296, wherein the amino acid sequence corresponds to positions 587-595 of SEQ ID NO: 5, 8, or 3636.
298. The isolated AAV particle of any one of embodiments 1-297, wherein the AAV capsid variant comprises one or two, but no more than three different amino acids (e.g., substitutions, e.g. conservative substitutions) relative to the amino acid sequence of PLNGAVHLYAQAQLSPVKN (SEQ ID NO: 566), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
299. The isolated AAV particle of any one of embodiments 1-298, wherein the AAV capsid variant comprises one or two, but no more than three different amino acids (e.g., substitutions, e.g., conservative substitutions) relative to the amino acid sequence of PLNGAVHLYAQAQLSPVKN (SEQ ID NO: 566), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
300. The isolated AAV particle of any one of embodiments 1-297, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises:
   (a) a VP1 protein comprising the amino acid sequence of SEQ ID NO: 5, 8, 138, or 3636;
   (b) a VP2 protein comprising the amino acid sequence of positions 138-736 of SEQ ID NO: 138 or positions 138-743 of SEQ ID NO: 5, 8, or 3636;
   (c) a VP3 protein comprising the amino acid sequence of positions 203-736 of SEQ ID NO: 138 or positions 203-743 of SEQ ID NO: 5, 8, or 3636; or
   (d) an amino acid sequence with at least 90% (e.g., at least about 95, 96, 97, 98, or 99%) sequence identity to any of the amino acid sequences in (a)-(c).
301. The isolated AAV particle of any one of embodiments 1-297 or 300, wherein the AAV capsid variant comprises one or two, but no more than three different amino acids (e.g., substitutions, e.g., conservative substitutions) relative to the amino acid sequence of PLNGAVHLYAQAQTGWVPN (SEQ ID NO: 314), wherein the AAV capsid variant comprises the amino acid sequence of any one of SEQ ID NOs: 5, 8, 138, or 3636 or an amino acid sequence with at least 90% (e.g., at least about 95, 96, 97, 98, or 99%) sequence identity to any one of SEQ ID NOs: 5, 8, 138, or 3636.
302. The isolated AAV particle of any one of embodiments 298-301, wherein the amino acid sequence is present immediately subsequent to position 586, numbered according to any one of SEQ ID NO: 5, 8, 138, or 3636, optionally wherein the amino acid replaces positions 587-598, numbered according to SEQ ID NO: 138.
303. The isolated AAV particle of any one of embodiments 297-302, wherein the amino acid sequence corresponds to positions 587-605 of SEQ ID NO: 5 or 8.
304. The isolated AAV particle of any one of the preceding embodiments, wherein the amino acid sequence is present in loop VIII.
305. The isolated AAV particle of any one of embodiments 3-5, or 270-304, wherein the amino acid sequence is present immediately subsequent to position 586, 587, 588, 589, 590, 591, 592, 593, 594, or 595, relative to a reference sequence numbered according to the amino acid sequence of SEQ ID NO: 138.
306. The isolated AAV particle of any one of embodiments 270-305, wherein the amino acid sequence is present immediately subsequent to position 586, relative to a reference sequence numbered according to the amino acid sequence of SEQ ID NO: 138.
307. The isolated AAV particle of any one of embodiments 270-306, wherein the amino acid sequence replaces positions 587 and 588 (e.g., A587 and Q588), numbered according to SEQ ID NO: 138
308. The isolated AAV particle of any one of embodiments 270-305, wherein the amino acid sequence is present immediately subsequent to position 588, relative to a reference sequence numbered according to the amino acid sequence of SEQ ID NO: 138.
309. The isolated AAV particle of any one of embodiments 270-305, wherein the amino acid sequence is present immediately subsequent to position 592, relative to a reference sequence numbered according to the amino acid sequence of SEQ ID NO: 138.
310. The isolated AAV particle of any one of embodiments 270-305, wherein the amino acid sequence is present immediately subsequent to position 595, relative to a reference sequence numbered according to the amino acid sequence of SEQ ID NO: 138.
311. The isolated AAV particle of any one of embodiments 272-310, wherein the AAV capsid variant further comprises:
   (i) one, two, three, or all of an amino acid other than A at position 589, an amino acid other than Q at position 590, an amino acid other than A at position 591, and/or an amino acid other than Q at position 592;
   (ii) one, two, or all of an amino acid other than T at position 593, an amino acid other than G at position 594, and/or an amino acid other than W at position 595, relative to a reference sequence numbered according to the amino acid sequence of SEQ ID NO: 138
   (iii) one, two, or all of an amino acid other than V at position 596, an amino acid other than Q at position 597, and/or an amino acid other than N at position 598, relative to a reference sequence numbered according to the amino acid sequence of SEQ ID NO: 138.
312. The isolated AAV particle of any one of the preceding embodiments, wherein the AAV capsid variant further comprises an amino acid other than A at position 587 and an amino acid other than Q at position 588.
313. The isolated AAV particle of any one of embodiments 270-313, wherein the AAV capsid variant comprises the amino acid P at position 587 the amino acid L at position 588, and the amino acid sequence NGAVHLY (SEQ ID NO: 3689), which is present immediately subsequent to position 588, corresponding to or numbered according to SEQ ID NO: 5, 8, 138, or 3636.
314. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises the amino acid P at position 587 the amino acid L at position 588, and the amino acid sequence NGAVHLY (SEQ ID NO: 3689) present immediately subsequent to position 588, corresponding to or numbered according to SEQ ID NO: 5, 8, 138, or 3636.
315. The isolated AAV particle of any one of embodiments 270-314, wherein the AAV capsid variant further comprises:
   (i) one, two, or all of an amino acid other than T at position 593, an amino acid other than G at position 594, and/or an amino acid other than W at position 595, numbered according to the amino acid sequence of SEQ ID NO: 138; or
   (ii) one, two, or all of an amino acid other than T at position 600, an amino acid other than G at position 601, and/or an amino acid other than W at position 602, numbered according to the amino acid sequence of SEQ ID NO: 5, 8, or 3636.
316. The isolated AAV particle of any one of embodiments 270-315, wherein the AAV capsid variant further comprises:
   (i) an amino acid other than T at position 593, an amino acid other than G at position 594, and an amino acid other than W at position 595, numbered according to the amino acid sequence of SEQ ID NO: 138; or
   (ii) an amino acid other than T at position 600, an amino acid other than G at position 601, and an amino acid other than W at position 602, numbered according to the amino acid sequence of SEQ ID NO: 5, 8, 3636.
317. The isolated AAV particle of any one of embodiments 270-316, wherein the AAV capsid variant further comprises:
   (i) one, two, or all of the amino acid L at position 593, the amino acid S at position 594, and/or the amino acid P at position 595, numbered according to the amino acid sequence of SEQ ID NO: 138;
   (ii) one, two, or all of the amino acid L at position 600, the amino acid S at position 601, and/or the amino acid P at position 602, numbered according to the amino acid sequence of SEQ ID NO: 5, 8, or 3636.
318. The isolated AAV particle of any one of embodiments 270-317, wherein the AAV capsid variant further comprises:
   (i) the amino acid L at position 593, the amino acid S at position 594, and the amino acid P at position 595, numbered according to the amino acid sequence of SEQ ID NO: 138; or
   (ii) the amino acid L at position 600, the amino acid S at position 601, and the amino acid P at position 602, numbered according to the amino acid sequence of SEQ ID NO: 5, 8, or 3636.
319. The isolated AAV particle of any one of embodiments 270-318, wherein the AAV capsid variant further comprises:
   (i) one, two, or all of an amino acid other than V at position 596, an amino acid other than Q at position 597, and/or an amino acid other than N at position 598, numbered according to the amino acid sequence of SEQ ID NO: 138;
   (ii) one, two, or all of an amino acid other than V at position 603, an amino acid other than Q at position 604, and/or an amino acid other than N at position 605, numbered according to the amino acid sequence of SEQ ID NO: 5, 8, 3636.
320. The isolated AAV particle of any one of embodiments 270-319, wherein the AAV capsid variant further comprises an amino acid other than Q at position 597 numbered according to the amino acid sequence of SEQ ID NO: 138 or at position 604 numbered according to SEQ ID NO: 5, 8, or 3636.
321. The isolated AAV particle of any one of embodiments 270-320, wherein the AAV capsid variant further comprises the amino acid P at position 597 numbered according to the amino acid sequence of SEQ ID NO: 138 or at position 604 numbered according to SEQ ID NO: 5, 8, or 3636.
322. The isolated AAV particle of any one of embodiments 270-320, wherein the AAV capsid variant further comprises the amino acid K at position 597 numbered according to the amino acid sequence of SEQ ID NO: 138 or at position 604 numbered according to SEQ ID NO: 5, 8, or 3636.
323. The isolated AAV particle of any one of embodiments 270-320, wherein the AAV capsid variant further comprises the amino acid E or H at position 597 numbered according to the amino acid sequence of SEQ ID NO: 138 or at position 604 numbered according to SEQ ID NO: 5, 8, or 3636.
324. The isolated AAV particle of any one of embodiments 270-320 or 322, wherein the AAV capsid variant further comprises the amino acid L at position 593, the amino acid S at position 594, the amino acid P at position 595, and the amino acid K at position 597, numbered according to the amino acid sequence of SEQ ID NO: 138.
325. The isolated AAV particle of any one of embodiments 270-320, 322 or 324, wherein the AAV capsid variant further comprises the amino acid L at position 600, the amino acid S at position 601, the amino acid P at position 602, and the amino acid K at position 604, corresponding to or numbered according to the amino acid sequence of SEQ ID NO: 8 or 3636.
326. The isolated AAV particle of any one of embodiments 270-320, 322, 324 or 325, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
327. The isolated AAV particle of any one of embodiments 270-320, 322, 324-326, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
328. The isolated AAV particle of any one of embodiments 270-320, 322, 234-327, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
329. The isolated AAV particle of any one of embodiments 270-321, wherein the AAV capsid variant further comprises the amino acid P at position 597, numbered according to the amino acid sequence of SEQ ID NO: 138.
330. The isolated AAV particle of any one of embodiments 270-321 or 329, wherein the AAV capsid variant further comprises the amino acid P at position 604, corresponding to or numbered according to the amino acid sequence of SEQ ID NO: 5 or 3636.
331. The isolated AAV particle of any one of embodiments 270-321, 329 or 330, wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
332. The isolated AAV particle of any one of embodiments 270-321 or 329-331, wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
333. The isolated AAV particle of any one of embodiments 270-321 or 329-332, wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 5 or 3636; and
   (ii) the amino acid P at position 604, corresponding to or numbered according to the amino acid sequence of SEQ ID NO: 5 or 3636.
334. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
335. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid L at position 593, the amino acid S at position 594, the amino acid P at position 595, and the amino acid K at position 597, numbered according to the amino acid sequence of SEQ ID NO: 138.
336. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 8 or 3636; and
   (ii) the amino acid L at position 600, the amino acid S at position 601, the amino acid P at position 602, and the amino acid K at position 604, corresponding to or numbered according to the amino acid sequence of SEQ ID NO: 8 or 3636.
337. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid sequence of PLNGAVHLY (SEQ ID NO: 3648), optionally where the amino acid sequence is present immediately subsequent to position 586 and replaces positions 587 and 588 (e.g., A587 and Q 588), numbered according to SEQ ID NO: 138; and
   (ii) the amino acid P at position 597, numbered according to the amino acid sequence of SEQ ID NO: 138.
338. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid P at position 587, the amino acid L at position 588, and the amino acid sequence NGAVHLY (SEQ ID NO: 3689), which is present immediately subsequent to position 588, numbered according to SEQ ID NO: 138; and
   (ii) the amino acid P at position 597, numbered according to the amino acid sequence of SEQ ID NO: 138.
339. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises:
   (i) the amino acid P at position 587, the amino acid L at position 588, and the amino acid sequence NGAVHLY (SEQ ID NO: 3689), which is present immediately subsequent to position 588, corresponding to or numbered according to SEQ ID NO: 5 or 3636; and
   (ii) the amino acid P at position 604, corresponding to or numbered according to the amino acid sequence of SEQ ID NO: 5 or 3636.
340. The isolated AAV particle of any one of the preceding embodiments, wherein the AAV capsid variant further comprises:
   (i) a modification, e.g., an insertion, substitution (e.g., conservative substitution), and/or deletion, in loop I, II, IV, and/or VI; and/or
   (ii) a substitution at position K449, e.g., a K449R substitution, numbered according to SEQ ID NO: 138.
341. The isolated AAV particle of any one of the preceding embodiments, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138.
342. The isolated AAV particle of any one of the preceding embodiments, wherein the AAV capsid variant comprises an amino acid sequence comprising at least one, two or three, but no more than 30, 20 or 10 different amino acids relative to the amino acid sequence of SEQ ID NO: 138.
343. The isolated AAV particle of any one of the preceding embodiments, wherein the AAV capsid variant comprises the amino acid sequence of SEQ ID NO: 138, or an amino acid sequence with at least 80% (e.g., at least about 85, 90, 95, 96, 97, 98, or 99%) sequence identity thereto.
344. The isolated AAV particle of any one of the preceding embodiments, wherein the AAV capsid variant comprises the amino acid sequence of SEQ ID NO: 138.
345. The isolated AAV particle of any one of the preceding embodiments, wherein the AAV capsid variant comprises an amino acid sequence encoded by the nucleotide sequence of SEQ ID NO: 137, or a sequence with at least 80% (e.g., at least about 85, 90, 95, 96, 97, 98, or 99%) sequence identity thereto.
346. The isolated AAV particle of any one of the preceding embodiments, wherein the nucleotide sequence encoding the capsid variant comprises the nucleotide sequence of SEQ ID NO: 137, or a sequence with at least 80% (e.g., at least about 85, 90, 95, 96, 97, 98, or 99%) sequence identity thereto.
347. The isolated AAV particle of any one of the preceding embodiments, wherein the AAV capsid variant comprises a VP1 protein, a VP2 protein, a VP3 protein, or a combination thereof.
348. The isolated AAV particle of any one of embodiments 1-347, wherein the AAV capsid variant comprises the amino acid sequence corresponding to positions 138-743, e.g., a VP2, of SEQ ID NO: 5, 8, or 3636, or a sequence with at least 80% (e.g., at least about 85, 90, 95, 96, 97, 98, or 99%) sequence identity thereto.
349. The isolated AAV particle of any one of embodiments 1-348, wherein the AAV capsid variant comprises the amino acid sequence corresponding to positions 203-743, e.g., a VP3, of SEQ ID NO: 5, 8, or 3636, or a sequence with at least 80% (e.g., at least about 85, 90, 95, 96, 97, 98, or 99%) sequence identity thereto.
350. The isolated AAV particle of any one of embodiments 1-349, wherein the AAV capsid variant comprises the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto.
351. The isolated AAV particle of any one of embodiments 1-350, wherein the AAV capsid variant comprises the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto.
352. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises the amino acid sequence of SEQ ID NO: 5.
353. The isolated AAV particle of embodiment 352, wherein the nucleotide sequence encoding the AAV capsid variant comprises the nucleotide sequence of SEQ ID NO: 4.
354. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises the amino acid sequence of SEQ ID NO: 8.
355. The isolated AAV particle of embodiment 354, wherein the nucleotide sequence encoding the AAV capsid variant comprises the nucleotide sequence of SEQ ID NO: 7.
356. An isolated, e.g., recombinant, AAV particle comprising an AAV capsid variant and a nucleic acid comprising a transgene encoding a SOD1 targeting polynucleotide (e.g., a SOD1 targeting RNAi), wherein the AAV capsid variant comprises the amino acid sequence of SEQ ID NO: 3636.
357. The isolated AAV particle of embodiment 356, wherein the nucleotide sequence encoding the AAV capsid variant comprises the nucleotide sequence of SEQ ID NO: 3623.
358. The isolated AAV particle of any one of the preceding embodiments, wherein the AAV capsid variant does not comprise:
   (i) the amino acid sequence of TLAVPFK (SEQ ID NO: 1262) present immediately subsequent to position 588, numbered according to SEQ ID NO: 138;
   (ii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to 594 numbered relative to SEQ ID NO: 138, of any the amino acid sequences provided in Table 1 of WO2020223276, the contents of which are hereby incorporated by reference in their entirety; or
   (iii) an amino acid sequence present immediately subsequent to position 586 to 599, e.g., 586 to 594, 587 to 595, 588 to 596, 589 to 597, 590 to 598 numbered relative to SEQ ID NO: 138, having at least 5 consecutive amino acids corresponding to positions 586 to Macaca fascicularis 594 numbered relative to SEQ ID NO: 138, of any SEQ ID NOs: 1, 3, 12, 13, or 138.
359. The isolated AAV particle of any one of the preceding embodiments, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
360. The isolated AAV particle of any one of embodiments 15-359, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 3636.
361. The isolated AAV particle of any one of embodiments 15-360, wherein the AAV capsid variant is enriched at least about 1.1, 1.2, 1.3, 1.4, 1.5, 1.6, 1.7, 1.8, 1.9, 2, 2.5, 3, 3.5, 4, 4.5, 5, 5.5, or 6-fold, in the brain compared to a reference sequence of SEQ ID NO: 3636, e.g., when measured by an assay as described in Example 18.
362. The isolated AAV particle of any one of embodiments 71, 74, 75, 272, 277, 280, 285, 286, 291 or 304-360, wherein the AAV capsid variant results in greater than 1, 2, 5, 10, 20, 30, 40, 50, or 100 reads per sample, e.g., when analyzed by an NGS sequencing assay, e.g., as described in Example 18.
363. The isolated AAV particle of any one of embodiments 1-362, wherein the AAV capsid variant is enriched in the brain of at least two to three species, e.g., a non-human primate and rodent (e.g., mouse), e.g., as compared to a reference sequence of SEQ ID NO: 138.
364. The isolated AAV particle of embodiment 363, wherein the at least two to three species are Macaca fascicularis, Chlorocebus sabaeus, Callithrix jacchus, and/or mouse (e.g., BALB/c mice).
365. The isolated AAV particle of any one of the preceding embodiments, wherein the AAV capsid variant further comprises a modification, e.g., substitution (e.g., conservative substitution), insertion, or deletion, that results in one, two, three or all of: (1) reduced tropism in the liver; (2) de-targeted expression in the liver; (3) reduced activity in the liver; and/or (4) reduced binding to galactose.
366. The AAV capsid variant of any one of the preceding embodiments, wherein the AAV capsid variant further comprises:
   (i) a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N4981), W503 (e.g., W530R or W530A), L620 (e.g., L620F), or a combination thereof, relative to a reference sequence numbered according to SEQ ID NO: 138; or
   (ii) one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138.
367. The AAV particle of any one of the above embodiments, wherein the SOD1 targeting polynucleotide is a single-stranded antisense RNA molecule, a single-stranded siRNA, or a double-stranded RNA (e.g., a siRNA duplex) that inhibits expression of SOD1.
368. The AAV particle of embodiment 367, wherein the SOD1 targeting polynucleotide is a siRNA duplex comprising a sense strand and an antisense strand.
369. The AAV particle of any one of the above embodiments, wherein SOD1 comprises a wild type SOD1 gene, mRNA, and/or protein; a mutated SOD1 gene, mRNA, and/or protein comprising at least one mutation; or a combination thereof.
370. The AAV particle of embodiments 368 or 369, wherein the antisense strand comprises a region that is substantially complementary (e.g., at least 70%, 75%, 80%, 85%, 90%, 95%, 99% or 100% complementary) to at least part of an mRNA transcript of a SOD1 gene (e.g., a human SOD1 gene).
371. The AAV particle of embodiments 368 or 369, wherein the antisense strand comprises a region that is substantially complementary (e.g., at least 70%, 75%, 80%, 85%, 90%, 95%, 99% or 100% complementary) to at least part of a SOD1 gene (e.g., a human SOD1 gene).
372. The AAV particle of embodiment 371, wherein the antisense strand comprises a region that is substantially complementary (e.g., at least 70%, 75%, 80%, 85%, 90%, 95%, 99% or 100% complementary) to a mRNA coding region of a SOD1 gene, or a non-coding region of a SOD1 gene.
373. The AAV particle of any one of embodiments 369-372, wherein the region that is substantially complementary is 30 nucleotides or less (e.g., 19-24 nucleotides in length).
374. The AAV particle of any one of embodiments 368-373, wherein the siRNA duplex ranges from 9 to 36 nucleotides in length (e.g., from 15 to 30 nucleotides in length, from 15 to 25 nucleotides in length, or from 17 to 22 nucleotides in length).
375. The AAV particle of any one of embodiments 368-374, wherein the siRNA duplex comprises:
   (i) a sense strand sequence comprising at least 15 (e.g., at least 16, 17, 18, 19, 20, 21, 22, or 23) contiguous nucleotides differing by no more than 3 (e.g., by no more than 0, 1 or 2) nucleotides from a sense sequence in Table 10; and
   (ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from an antisense sequence in Table 10.
376. The AAV particle of any one of embodiments 368-374, wherein the siRNA duplex comprises:
   (i) a sense strand sequence from Table 10, or a nucleotide at least 80%, 81%, 82%, 83%, 84%, 85%, 86%, 87%, 88%, 89%, 90%, 91%, 92%, 93%, 94%, 95%, 96%, 97%, 98% or 99%, identical to a sense strand sequence in Table 10; and
   (ii) an antisense strand sequence from Table 10, or a nucleotide at least 80%, 81%, 82%, 83%, 84%, 85%, 86%, 87%, 88%, 89%, 90%, 91%, 92%, 93%, 94%, 95%, 96%, 97%, 98% or 99%, identical to an antisense strand sequence in Table 10.
377. The AAV particle of any one of embodiments 368-376, wherein the siRNA duplex is selected from the group consisting of siRNA duplex ID Nos. D-2968, D-2959, or D-4012, or a siRNA duplex at least 80%, 81%, 82%, 83%, 84%, 85%, 86%, 87%, 88%, 89%, 90%, 91%, 92%, 93%, 94%, 95%, 96%, 97%, 98% or 99%, identical to siRNA duplex ID Nos. D-2968, D-2959, or D-4012.
378. The AAV particle of any one of embodiments 368-377, wherein the siRNA duplex comprises:
   (i) a sense strand sequence comprising at least 15 (e.g., at least 16, 17, 18, 19, 20, 21, 22, or 23) contiguous nucleotides differing by no more than 3 (e.g., by no more than 0, 1 or 2) nucleotides from SEQ ID NO: 2525, 2376, or 2381; and
   (ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from SEQ ID NO: 2507, 2365, or 2363.
379. The AAV particle of any one of embodiments 368-378, wherein the siRNA duplex comprises:
   (i) a sense strand sequence comprising SEQ ID NO: 2525, or a nucleotide differing by no more than 3 (e.g., by no more than 0, 1 or 2) nucleotides from SEQ ID NO: 2525, and an antisense strand sequence comprising SEQ ID NO: 2507, or a nucleotide differing by no more than 3 (e.g., by no more than 0, 1 or 2) nucleotides from SEQ ID NO: 2507;
   (ii) a sense strand sequence comprising SEQ ID NO: 2376, or a nucleotide differing by no more than 3 (e.g., by no more than 0, 1 or 2) nucleotides from SEQ ID NO: 2376, and an antisense strand sequence comprising SEQ ID NO: 2365, or a nucleotide differing by no more than 3 (e.g., by no more than 0, 1 or 2) nucleotides from SEQ ID NO: 2365; and/or
   (iii) a sense strand sequence comprising SEQ ID NO: 2381, or a nucleotide differing by no more than 3 (e.g., by no more than 0, 1 or 2) nucleotides from SEQ ID NO: 2381, and an antisense strand sequence comprising SEQ ID NO: 2363, or a nucleotide differing by no more than 3 (e.g., by no more than 0, 1 or 2) nucleotides from SEQ ID NO: 2363.
380. The AAV particle of embodiment 379, wherein the siRNA duplex comprises:
   (i) a sense strand sequence comprising SEQ ID NO: 2525, and an antisense strand sequence comprising SEQ ID NO: 2507;
   (ii) a sense strand sequence comprising SEQ ID NO: 2376, and an antisense strand sequence comprising SEQ ID NO: 2365; and/or
   (iii) a sense strand sequence comprising SEQ ID NO: 2381, and an antisense strand sequence comprising SEQ ID NO: 2363.
381. The AAV particle of embodiment 380, wherein the siRNA duplex comprises a sense strand sequence comprising SEQ ID NO: 2525, and an antisense strand sequence comprising SEQ ID NO: 2507.
382. The AAV particle of any one of embodiments 368-381, wherein the sense strand sequence and the antisense strand sequence comprise a region of complementarity, wherein the region of complementarity is between 17-20 nucleotides in length.
383. The AAV particle of any one of embodiments 368-382, wherein at least one of the sense strand sequence and the antisense strand sequence comprise a 3′ overhang of at least 1, or 2 nucleotides.
384. The AAV particle of any one of embodiments 368-383, wherein sense strand and/or antisense strand are at least 20, 21, or 22 nucleotides in length.
385. The AAV particle of any one of embodiments 367-384, wherein the siRNA duplex is encoded by a modulatory polynucleotide comprising:
   (i) a 5′ flanking region;
   (ii) a loop region; and/or
   (iii) a 3′ flanking region.
386. The AAV particle of embodiment 384, wherein the modulatory polynucleotide comprises:
   (i) a 5′ flanking region comprising a nucleotide sequence of SEQ ID NO: 2547, 2548, 2549, or 5014, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2547, 2548, 2549, or 5014;
   (ii) a loop region comprising a nucleotide sequence of SEQ ID NO: 2550, 2551, 2552, or 2553, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, or four, modifications, but no more than six modifications of SEQ ID NO: 2550, 2551, 2552, or 2553; and/or
   (iii) a 3′ flanking region comprising a nucleotide sequence of SEQ ID NO: 2554, 2555, 2556, 2557, or 2558, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2554, 2555, 2556, 2557, or 2558.
387. The AAV particle of any one of embodiments 384-385, wherein:
   (i) the 5′ flanking region comprises a nucleotide sequence of SEQ ID NO: 2547, or 2549, a nucleotide sequence having at least one, two, three, four, five, six, or seven modifications, but no more than ten modifications of SEQ ID NO: 2547, or 2549;
   (ii) the loop region comprises a nucleotide sequence of SEQ ID NO: 2550, 2551, or 2552, or a nucleotide sequence having at least one, two, three, or four, modifications, but no more than six modifications of SEQ ID NO: 2550, 2551, or 2552; and/or
   (iii) 3′ flanking region comprises a nucleotide sequence of SEQ ID NO: 2554, 2555, or 2558, or a nucleotide sequence having at least one, two, three, four, five, six, or seven modifications, but no more than ten modifications of SEQ ID NO: 2554, 2555, or 2558.
388. The AAV particle of embodiment 387, wherein:
   (i) the 5′ flanking region comprises a nucleotide sequence of SEQ ID NO: 2547;
   (ii) the loop region comprises a nucleotide sequence of SEQ ID NO: 2550; and/or
   (iii) 3′ flanking region comprises a nucleotide sequence of SEQ ID NO: 2554.
389. The AAV particle of any one of embodiments 367-388, wherein the siRNA duplex is encoded by a nucleotide sequence of any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 70% (e.g., at least 75%, 80%, 85%, 90%, 92%, 95%, 97%, 98%, or 99%) identical thereto.
390. The AAV particle of any one of embodiments 367-389, wherein the siRNA duplex is encoded by a nucleotide sequence of any one of SEQ ID NOs: 2562, 2579, or 5022, or a nucleotide sequence at least 70% (e.g., at least 75%, 80%, 85%, 90%, 92%, 95%, 97%, 98%, or 99%) identical thereto.
391. The AAV particle of any one of embodiments 367-390, wherein the nucleotide sequence encoding the siRNA duplex comprises the nucleotide sequence of SEQ ID NO: 2562 or a nucleotide sequence at least 70% (e.g., at least 75%, 80%, 85%, 90%, 92%, 95%, 97%, 98%, or 99%) identical thereto.
392. The isolated AAV particle of any one of the preceding embodiments, which comprises a viral genome comprising a promoter operably linked to the nucleic acid sequence encoding the SOD1 targeting polynucleotide (e.g., a SOD1 targeting RNA agent, e.g., a siRNA duplex)
393. The isolated AAV particle of embodiment 392, wherein the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), phosphoglycerate kinase (PGK), insulin, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
394. The isolated AAV particle of embodiment 393, wherein the promoter is an EF-la promoter variant, e.g., a truncated EF-la promoter, a CBA promoter variant, e.g., a truncated CBA promoter, an insulin promoter variant, e.g., a truncated insulin promoter, or a SYN promoter variant, e.g., a truncated SYN promoter.
395. The isolated AAV particle of any one of embodiments 392-394, wherein the promoter comprises the nucleotide sequence of any one of SEQ ID NOs: 4000-4026, a nucleotide sequence comprising at least one, two, or three but no more than four modifications, e.g., substitutions (e.g., conservative substitutions), insertions, or deletions, relative to the nucleotide sequence of SEQ ID NOs: 4000-4026, or a nucleotide sequence with at least 80% (e.g., 85%, 90%, 95%, 96%, 97%, 98%, or 99%) sequence identity to any one of SEQ ID NOs: 4000-4026.
396. The isolated AAV particle of embodiment 392, wherein the promoter is a H1 promoter comprising SEQ ID NO: 128, or a nucleotide sequence with at least 80% (e.g., 85%, 90%, 95%, 96%, 97%, 98%, or 99%) sequence identity to SEQ ID NO: 128.
397. The isolated AAV particle of any one of embodiments 392-396, wherein the viral genome further comprises a first exon, a second exon, or both a first exon and second exon.
398. The isolated AAV particle of any one of embodiments 397, wherein the first or second exon comprises the nucleotide sequence of any one of SEQ ID NO: 4045-4048, a nucleotide sequence comprising at least one, two, or three but no more than four modifications, e.g., substitutions, insertions, or deletions, relative to the nucleotide sequence of any one of SEQ ID NO: 4045-4048, or a nucleotide sequence with at least 80% (e.g., 85%, 90%, 95%, 96%, 97%, 98%, or 99%) sequence identity to any one of SEQ ID NO: 4045-4048.
399. The isolated AAV particle of any one of embodiments 397 or 398, wherein the viral genome comprises both a first exon and a second exon, wherein:
   (a) the first exon comprises the nucleotide sequence of SEQ ID NO: 4045 or 4046, a nucleotide sequence comprising at least one, two, or three but no more than four modifications, e.g., substitutions, insertions, or deletions, relative to the nucleotide sequence of SEQ ID NO: 4045 or 4046, or a nucleotide sequence with at least 80% (e.g., 85%, 90%, 95%, 96%, 97%, 98%, or 99%) sequence identity to SEQ ID NO: 4045 or 4046; and
   (b) the second exon comprises the nucleotide sequence of SEQ ID NO: 4047 or 4048, a nucleotide sequence comprising at least one, two, or three but no more than four modifications, e.g., substitutions, insertions, or deletions, relative to the nucleotide sequence of SEQ ID NO: 4047 or 4048, or a nucleotide sequence with at least 80% (e.g., 85%, 90%, 95%, 96%, 97%, 98%, or 99%) sequence identity to SEQ ID NO: 4047 or 4048.
400. The isolated AAV particle of any one of embodiments 397-399, wherein the first exon, second exon, or both are codon optimized.
401. The isolated AAV particle of any one of embodiments 397-400, wherein one or more or all of the CpG sequences in the first exon, second exon, or both are depleted.
402. The isolated AAV particle of any one of embodiments 392-401, wherein the viral genome further comprises an intron.
403. The isolated AAV particle of embodiment 402, wherein the intron is located 3′ relative to the (a) 5′ITR, (b) promoter, and/or (c) first exon.
404. The isolated AAV particle of embodiment 402 or 403, wherein the intron is located 5′ relative to the (a) second exon, (b) modulatory polynucleotide, (c) poly A signal sequence, and/or (d) 3′ITR.
405. The isolated AAV particle of any one of embodiments 392-404, wherein the viral genome comprises a first exon, an intron, and a second exon, wherein the intron is located 3′ relative to the first exon and 5′ relative to the second exon.
406. The isolated AAV particle of any one of embodiments 402-405, wherein the intron comprises the nucleotide sequence of SEQ ID NO: 4044, a nucleotide sequence comprising at least one, two, or three but no more than four modifications, e.g., substitutions, insertions, or deletions, relative to the nucleotide sequence of SEQ ID NO: 4044, or a nucleotide sequence with at least 80% (e.g., 85%, 90%, 95%, 96%, 97%, 98%, or 99%) sequence identity to SEQ ID NO: 4044.
407. The isolated AAV particle of any one of embodiments 392-406, wherein the viral genome comprises a first exon, an intron, and second exon (“exon-intron-exon cassette”).
408. The isolated AAV particle of embodiment 407, wherein the exon-intron-exon cassette comprises the nucleotide sequence of SEQ ID NO: 4042 or 4043, a nucleotide sequence comprising at least one, two, or three but no more than four modifications, e.g., substitutions, insertions, or deletions, relative to the nucleotide sequence of SEQ ID NO: 4042 or 4043, or a nucleotide sequence with at least 80% (e.g., 85%, 90%, 95%, 96%, 97%, 98%, or 99%) sequence identity to SEQ ID NO: 4042 or 4043.
409. The isolated AAV particle of embodiment 407 or 408, wherein the exon-intron-exon cassette is positioned 3′ relative to the promoter, and 5′ relative to the nucleotide sequence encoding the modulatory polynucleotide (e.g., a SOD1 targeting RNA agent, e.g., a siRNA duplex).
410. The isolated AAV particle of any one of embodiments 407-409, wherein the nucleotide sequence encoding the modulatory polynucleotide (e.g., a SOD1 targeting RNA agent, e.g., a siRNA duplex) is located within (e.g., inserted into) the intron of the exon-intron-exon cassette.
411. The isolated AAV particle of any one of embodiments 407-410, wherein the nucleotide sequence encoding the modulatory polynucleotide (e.g., a SOD1 targeting RNA agent, e.g., a siRNA duplex) replaces a portion or all of the intron in the exon-intron-exon cassette.
412. The isolated AAV particle of any one of embodiments 392-411, wherein the viral genome further comprises a polyA signal sequence.
413. The isolated AAV particle of embodiment 412, wherein the polyA signal sequence comprises SEQ ID NO: 129 or 4027, or a nucleotide sequence with at least 80% (e.g., 85%, 90%, 95%, 96%, 97%, 98%, or 99%) sequence identity to SEQ ID NO: 129 or 4027.
414. The isolated AAV particle of any one of embodiments 392-413, wherein the viral genome further comprises an inverted terminal repeat (ITR) sequence.
415. The isolated AAV particle of any one of embodiments 392-414, wherein the viral genome comprises an ITR sequence positioned 5′ relative to the encoded SOD1 targeting polynucleotide.
416. The isolated AAV particle of any one of embodiments 392-415, wherein the viral genome comprises an ITR sequence positioned 3′ relative to the encoded SOD1 targeting polynucleotide.
417. The isolated AAV particle of any one of embodiments 392-416, wherein the viral genome comprises an ITR sequence positioned 5′ relative to the encoded payload and an ITR sequence positioned 3′ relative to the encoded SOD1 targeting polynucleotide.
418. The isolated AAV particle of embodiment 417, wherein the ITR sequence positioned 5′ relative to the encoded payload and an ITR sequence positioned 3′ relative to the payload, comprise SEQ ID NOs: 126, or 130, or a nucleotide sequence with at least 80% (e.g., 85%, 90%, 95%, 96%, 97%, 98%, or 99%) sequence identity thereto.
419. The isolated AAV particle of any one of embodiments 392-418, wherein the viral genome further comprises an enhancer, a Kozak sequence, an intron region, and/or an exon region.
420. The isolated AAV particle of any one of embodiments 392-419, wherein the viral genome comprises, from 5′ to 3′:
   (a) a 5′ ITR, e.g., a 5′ITR comprising the nucleotide sequence of SEQ ID NO: 126 or 130 or variant thereof,
   (b) a promoter, e.g., a promoter comprising the nucleotide sequence of any one of SEQ ID NOs: 4000-4026 or variant thereof,
   (c) an exon, e.g., an exon comprising the nucleotide sequence of any one of SEQ ID NOs: 4045-4048 or variant thereof,
   (d) a nucleotide sequence encoding a modulatory polynucleotide (e.g., a SOD1 targeting RNA agent, e.g., a siRNA duplex), e.g., a modulatory polynucleotide comprising the nucleotide sequence of any one of SEQ ID NOs: 2559-2579 and 5022 or variant thereof,
   (e) a poly A signal sequence, e.g., a poly A signal sequence comprising the nucleotide sequence of SEQ ID NO: 129 or 4027 or variant thereof, and
   (f) a 3′ITR, e.g., a 3′ITR comprising the nucleotide sequence of SEQ ID NO: 126 or 130 or variant thereof,
   wherein the variant of any of (a)-(f) comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity, relative to the reference sequence, e.g., wherein the reference sequence is a wild-type sequence.
421. The isolated AAV particle of any one of embodiments 392-420, wherein the viral genome comprises, from 5′ to 3′:
   (a) a 5′ ITR, e.g., a 5′ITR comprising the nucleotide sequence of SEQ ID NO: 126 or 130 or variant thereof,
   (b) a promoter, e.g., a promoter comprising the nucleotide sequence of any one of SEQ ID NOs: 4000-4026 or variant thereof,
   (c) a first exon, e.g., a first exon comprising the nucleotide sequence of any one of SEQ ID NOs: 4045-4048 or variant thereof,
   (d) an intron, e.g., an intron comprising the nucleotide sequence of SEQ ID NO: 4044 or variant thereof,
   (e) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
   (f) a poly A signal sequence, e.g., a poly A signal sequence comprising the nucleotide sequence of SEQ ID NO: 129 or 4027 or variant thereof, and
   (g) a 3′ITR, e.g., a 3′ITR comprising the nucleotide sequence of SEQ ID NO: 126 or 130 or variant thereof,
   wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.
422. The isolated AAV particle of any one of embodiments 392-421, wherein the viral genome comprises, from 5′ to 3′:
   (a) a 5′ ITR, e.g., a 5′ITR comprising the nucleotide sequence of SEQ ID NO: 126 or 130 or variant thereof,
   (b) a promoter, e.g., a promoter comprising the nucleotide sequence of any one of SEQ ID NOs: 4000-4026 or variant thereof,
   (c) a first exon, e.g., a first exon comprising the nucleotide sequence of any one of SEQ ID NOs: 4045-4048 or variant thereof,
   (d) an intron, e.g., an intron comprising the nucleotide sequence of SEQ ID NO: 4044 or variant thereof,
   (e) a second exon, e.g., a second exon comprising the nucleotide sequence of any one of SEQ ID NOs: 4045-4048 or variant thereof,
   (f) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
   (g) a poly A signal sequence, e.g., a poly A signal sequence comprising the nucleotide sequence of SEQ ID NO: 129 or 4027 or variant thereof, and
   (h) a 3′ITR, e.g., a 3′ITR comprising the nucleotide sequence of SEQ ID NO: 126 or 130 or variant thereof,
   wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.
423. The isolated AAV particle of any one of embodiments 392-422, wherein the viral genome comprises the nucleotide sequence of any one of SEQ ID NOs: 4028-4041, a nucleotide sequence differing by no more than 15, 14, 13, 12, 11, 10, 9, 8, 7, 6, 5, 4, 3, 2 or 1 nucleotides, e.g., substitutions, insertions, or deletions, relative to any one of SEQ ID NOs: 4028-4041, or a nucleotide sequence with at least 70%, 75%, 80%, 85%, 90%, 95%, 99% or 100% identity to any one of SEQ ID NOs: 4028-4041.
424. The isolated AAV particle of any one of embodiments 392-423, wherein the viral genome further comprises a nucleotide sequence encoding a miR binding site, e.g., a miR binding site that modulates, e.g., reduces, expression of the SOD1 targeting polynucleotide encoded by the viral genome in a cell or tissue where the corresponding miRNA is expressed.
425. The isolated AAV particle of embodiment 424, wherein the encoded miRNA binding site is complementary, e.g., fully complementary or partially complementary, to a miRNA expressed in a cell or tissue of the DRG, liver, heart, hematopoietic, or a combination thereof.
426. The isolated AAV particle of embodiment 425 or 426, wherein the encoded miR binding site modulates, e.g., reduces, expression of the encoded SOD1 targeting polynucleotide in a cell or tissue of the DRG, liver, heart, hematopoietic lineage, or a combination thereof.
427. The isolated AAV particle of any one of embodiments 424-426, wherein the viral genome comprises at least 1-5 copies of the encoded miR binding site, e.g., at least 1, 2, 3, 4, or 5 copies.
428. The isolated AAV particle of any one of embodiments 424-427, wherein the viral genome comprises at least 3 copies of an encoded miR binding sites, optionally wherein all three copies comprise the same miR binding site, or at least one, two, three, or all of the copies comprise a different miR binding site.
429. The isolated AAV particle of embodiment 428, wherein the at least 3 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
430. The isolated AAV particle of any one of embodiments 324-329, wherein the viral genome comprises at least 4 copies of an encoded miR binding site, optionally wherein all four copies comprise the same miR binding site, or at least one, two, three, or all of the copies comprise a different miR binding site.
431. The isolated AAV particle of embodiment 430, wherein the at least 4 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
432. The isolated AAV particle of any one of embodiments 424-431, wherein the encoded miR binding site comprises a miR122 binding site, a miR183 binding site, a miR-1 binding site, a miR-142-3p, or a combination thereof, optionally wherein:
   (i) the encoded miR122 binding site comprises the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
   (ii) the encoded miR183 binding site comprises the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
   (iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679; and/or
   (iv) the encoded miR-142-3p binding site comprises the nucleotide sequence of SEQ ID NO: 1869, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1869.
433. The isolated AAV particle of any one of embodiments 392-432, wherein the viral genome comprises an encoded miR122 binding site.
434. The isolated AAV particle of any one of embodiments 392-433, wherein the viral genome comprises at least 1-5 copies, e.g., 1, 2, or 3 copies of a miR122 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
435. The isolated AAV particle of any one of embodiments 432-434, wherein the encoded miR122 binding site comprises the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865.
436. The isolated AAV particle of any one of embodiments 392-435, wherein the viral genome comprises:
(A) (i) a first encoded miR122 binding site comprising the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865;
   (ii) a first spacer comprising the nucleotide sequence SEQ ID NO: 1848, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (iii) a second encoded miR122 binding site comprising the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865; or
(B) (i) a first encoded miR122 binding site comprising the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to SEQ ID NO: 1848;
   (iii) a second encoded miR122 binding site comprising the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865;
   (iv) a second spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (v) a third encoded miR122 binding site comprising the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1865.
437. The isolated AAV particle of any one of embodiments 392-436, wherein the viral genome comprises an encoded miR183 binding site.
438. The isolated AAV particle of any one of embodiments 392-437, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.
439. The isolated AAV particle of embodiment 438, wherein the encoded miR183 binding site comprises the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847.
440. The isolated AAV particle of any one of embodiments 392-439, wherein the viral genome comprises:
(A) (i) a first encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (iii) a second encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847; or
(B) (i) a first encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847;
   (ii) a first spacer comprising the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA;
   (iii) a second encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847;
   (iv) a second spacer comprising the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; and
   (v) a third encoded miR183 binding site comprising the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1847.
441. The isolated AAV particle of any one of embodiments 392-340, wherein the viral genome comprises an encoded miR122 binding site and a miR-1 binding site.
442. The isolated AAV particle of any one of embodiments 392-441, wherein the viral genome is single stranded or self-complementary.
443. The isolated AAV particle of any one of embodiments 392-442, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein.
444. The isolated AAV particle of embodiment 443, wherein the Rep78 protein, the Rep68 protein, the Rep52 protein, and/or the Rep40 protein are encoded by at least one Rep gene.
445. A cell, e.g., a host cell, comprising the AAV particle of any one of the preceding embodiments.
446. The cell of embodiment 445, wherein the cell is a mammalian cell or an insect cell.
447. The cell of embodiment 445 or 446, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
448. The cell of any one of embodiments 445-447, wherein the cell is a neuron, a sensory neuron, a motor neuron, an astrocyte, or a glial cell.
449. A method of making the AAV particle of any one of embodiments 1-444, comprising:
   (i) providing a host cell comprising a viral genome; and
   (ii) incubating the host cell under conditions suitable to enclose the viral genome in an AAV capsid variant, e.g., an AAV capsid variant described herein;
   thereby making the AAV particle.
450. The method of embodiment 449, further comprising, prior to step (i), introducing a first nucleic acid molecule comprising the viral genome into the host cell.
451. The method of embodiment 449 or 450, wherein the host cell comprises a second nucleic acid encoding the capsid variant.
452. The method of embodiment 451, wherein the second nucleic acid molecule is introduced into the host cell prior to, concurrently with, or after the first nucleic acid molecule.
453. A pharmaceutical composition comprising the AAV particle of any one of embodiments 1-444, and a pharmaceutically acceptable excipient.
454. A method of delivering a payload to a cell or tissue (e.g., a CNS cell or a CNS tissue), comprising administering an effective amount of the pharmaceutical composition of embodiment 453, or the AAV particle of any one of embodiments 1-444.
455. The method of embodiment 454, wherein the cell is a cell a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
456. The method of embodiment 454 or 455, wherein the cell is a neuron, a sensory neuron, a motor neuron, or an astrocyte.
457. The method of any one of embodiments 454-456, wherein the cell or tissue is within a subject.
458. The method of embodiment 457, wherein the subject has, has been diagnosed with having, or is at risk of having a neurological, e.g., a neurodegenerative disorder.
459. The method of embodiment 457 or 458, wherein the subject has, has been diagnosed with having, or is at risk of having a disease associated with SOD1 expression or activity.
460. The method of any one of embodiments 457-459, wherein the subject has, has been diagnosed with having, or is at risk of having a disease associated with SOD1 expression or activity.
461. The method of any one of embodiments 457-460, wherein the subject has, has been diagnosed with having, or is at risk of having amyotrophic lateral sclerosis.
462. A method of treating a subject having or diagnosed with having a neurological disorder, e.g., a neurodegenerative disorder, comprising administering to the subject an effective amount of the pharmaceutical composition of embodiment 453, or the AAV particle of any one of embodiments 1-444.
463. A method of treating a subject having or diagnosed with having a disease related to expression of SOD1, comprising administering to the subject an effective amount of the pharmaceutical composition of embodiment 453, or the AAV particle of any one of embodiments 1-444.
464. A method of treating a subject having or diagnosed with a disease associated with SOD1 expression or activity, comprising administering to the subject an effective amount of the pharmaceutical composition of embodiment 453, or the AAV particle of any one of embodiments 1-444.
465. The method of any one of embodiments 458-464, wherein the neurological disorder, neurodegenerative disorder, or disease associated with SOD1 expression comprises amyotrophic lateral sclerosis (ALS).
466. The method embodiment 465, wherein the ALS is:
   (i) familial ALS;
   (ii) sporadic ALS;
   (iii) early stage ALS;
   (iv) middle stage ALS; and/or
   (v) late stage ALS.
467. The method of any one of embodiments 462-466, where treating comprises prevention of progression of the disease or disorder in the subject.
468. The method of any one of embodiments 457-467, wherein the subject is a human.
469. The method of any one of embodiments 465-468, wherein treatment comprises amelioration of a symptom of ALS in the subject.
470. The method of embodiment 469, wherein the symptom comprises motor neuron degeneration, muscle weakness, stiffness of muscles, muscle atrophy, muscle stiffness, fasciculation development, frontotemporal dementia, slurred speech, difficulty breathing, or a combination thereof.
471. The method of any one of embodiments 457-470, wherein the AAV particle is administered to the subject intravenously, via intra-cisterna magna injection (ICM), intracerebrally, intrathecally, intracerebroventricularly, via intraparenchymal administration, or intramuscularly.
472. The method of any one of embodiments 457-471, wherein the AAV particle is administered to the subject via focused ultrasound (FUS), e.g., coupled with the intravenous administration of microbubbles (FUS-MB), or MRI-guided FUS coupled with intravenous administration.
473. The method of any one of embodiments 457-472, wherein the AAV particle is administered to the subject intravenously.
474. The method of any one of embodiments 454-473, wherein administration of the AAV particle results in a decreased presence, level, and/or activity of a gene, mRNA, protein, or combination thereof.
475. The method of any one of embodiments 454-474, wherein administration of the AAV particle results in an increased presence, level, and/or activity of a gene, mRNA, protein, or a combination thereof.
476. The pharmaceutical composition of embodiment 453, or the AAV particle of any one of embodiments 1-444, for use in a method of delivering a payload to a cell or tissue.
477. The pharmaceutical composition of embodiment 453, or the AAV particle of any one of embodiments 1-444, for use in a method of treating a neurological disorder, a neurodegenerative disorder, a disease associated with SOD1 expression or activity.
478. The pharmaceutical composition of embodiment 453, or the AAV particle of any one of embodiments 1-444, for use in the manufacture of a medicament.
479. Use of the pharmaceutical composition of embodiment 453, or the AAV particle of any one of embodiments 1-444, in the manufacture of a medicament.
480. Use of the pharmaceutical composition of embodiment 453, or the AAV particle of any one of embodiments 1-444, in the manufacture of a medicament for treating a neurological disorder, a neurodegenerative disorder, a disease associated with SOD1 expression or activity.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIGS. 1A-1D show the expression of SOD1 mRNA from motor neuron pools in the lumbar L2 spinal cord. FIG. 1A is a graphical representation of the expression of SOD1 mRNA using Laser Capture Microdissection. Purified total RNA was reverse transcribed, cDNA pre-amplified, and quantified by qPCR with Taqman probe sets homologous to SOD1 and reference XPNPEP1 mRNA. FIG. 1B is an image of the hemi-sected cross-section of L2 spinal cord. FIG. 1C is an image of the pre-LCM motor neurons stained with cresyl violet stain (black arrows) and FIG. 1D is an image of the post-LCM depleted motor neurons (black arrows) in ventral horn.

FIG. 2 shows in-situ hybridization (ISH) using RNAscope technology to examine SOD1 expression in the motor neurons. The top panels show SOD1 mRNA (Dark grey spots above background) expression in the lumbar L2 spinal cord and in ventral horn in the inset images (see boxes). The bottom panels show ChAT staining in the motor neurons in the ventral horn. Nuclei were stained with hematoxylin in both top and bottom panels.

FIGS. 3A-3C show the digital spatial profile analysis of the Lumbar L2 spinal cord. FIG. 3A is a fluorescent image of the motor neurons labeled with ChAT, astrocytes with GFAP (highlighted areas along tissue edges) and nuclei stained with DAPI. ChAT positive motor neurons (see boxed area) were digital spatially profiled from a Lumbar L2 spinal cord sample using GeoMx Profiler (Nanostring) to study SOD1 and ChAT SEQ ID NO: 2562, mRNA expression. FIG. 3B are graphs depicting the counts and FIG. 3C (median value) are plots of the gene counts for SOD1 (top graph) and ChAT (bottom graph).

FIGS. 4A-4C are bar graphs depicting the distribution of an AAV particle with a VOY101 capsid and a viral genome (SEQ ID NO: 109, Table 15) encoding the SOD1 targeting modulatory polynucleotide, miR104-788.2 (Table 14), under the control of an H1 promoter (AAV_VOY101.SOD1) in the lower cervical spinal cord (FIG. 4A), lower thoracic spinal cord (FIG. 4B), and lower lumbar spinal cord (FIG. 4C) of SOD1^{G93A} transgenic mice. Groupings were as follows: Vehicle (Group V), 2E13 vg/kg (Group C), 6.3E12 (Group A), and 2E12 (Group B). **p<0.01; ***p<0.001; ****p<0.0001 (1-way ANOVA and Tukey's multiple comparisons).

FIGS. 5A-5C are bar graphs depicting the reduction in human SOD1 (hSOD1) mRNA after intravenous administration of an SOD1 miRNA-encoding AAV particle with a VOY101 capsid and a viral genome (SEQ ID NO: 109, Table 15) encoding the SOD1 targeting modulatory polynucleotide miR104-788.2 (SEQ ID NO: 2562, Table 14) under the control of an H1 promoter (AAV_VOY101.SOD1) in the lower cervical spinal cord (FIG. 5A), lower thoracic spinal cord (FIG. 5B), and lower lumbar spinal cord (FIG. 5C) in SOD1^{G93A} transgenic mice. Groupings were as follows: Vehicle (Group B), 2E13 vg/kg (Group C), 6.3E12 (Group D), and 2E12 (Group E). *p<0.05; **p<0.01; ***p<0.001 (1-way ANOVA and Tukey's multiple comparisons). hSOD1 mRNA levels are normalized to GAPDH+PPIA, and results are presented as values relative to the vehicle group (Group B).

FIGS. 6A-6C are graphs depicting correlations between hSOD1 mRNA levels and AAV particle distribution (VG/dg) in the lower cervical spinal cord (FIG. 6A), lower thoracic spinal cord (FIG. 6B), and lower lumbar spinal cord (FIG. 6C) of SOD1^{G93A} transgenic mice.

FIG. 7 shows the Neuroscore composite ranking of female and male wild-type mice administered vehicle and SOD1^{G93A} mice administered vehicle, 2E13 vg/kg, 6.3E12, or 2E12 vg/kg SOD1 miRNA AAV particle. The number of animals remaining in each group are also presented.

FIGS. 8A-8C are graphs depicting Kaplan-Meier survival curves for female (FIG. 8A), male (FIG. 8B), and all (FIG. 8C) wild-type mice administered vehicle and SOD1^{G93A} mice administered vehicle, 2E13 vg/kg, 6.3E12, or 2E12 vg/kg SOD1 miRNA AAV particle. **p=0.003; log-rank (Mantel-Cox) test.

FIGS. 9A and 9B are graphs depicting grip strength normalized to baseline for forelimbs (FIG. 9A) and all limbs combined (FIG. 9B) in female wild-type mice administered vehicle and SOD1^{G93A} mice administered vehicle, 2E13 vg/kg, 6.3E12, or 2E12 vg/kg SOD1 miRNA AAV particle. Baseline corresponds to the week prior to intravenous SOD1 miRNA AAV particle administration.

FIGS. 10A and 10B are graphs depicting grip strength normalized to baseline for forelimbs (FIG. 10A) and all limbs combined (FIG. 10B) in male wild-type mice administered vehicle and SOD1^{G93A} mice administered vehicle, 2E13 vg/kg, 6.3E12, or 2E12 vg/kg SOD1 miRNA AAV particle. Baseline corresponds to the week prior to intravenous SOD1 miRNA AAV particle administration.

FIG. 11 shows the distribution of an AAV particle with a wild-type AAV9 capsid or a TTD-001 capsid, in the spinal cord of NHPs in the Dorsal horn, Clarke's column, and ventral horn of NHPs by immunohistochemistry.

FIG. 12A is a graph showing the percentage of transduced cells (% HA positive cells) in various brain regions of NHPs following intravenous administration of a dose of 6.7e12 VG/kg of AAV particles comprising the TTD-001 capsid variant. FIG. 12B is a graph showing the percentage of transduced cells (% HA positive cells) in various brain regions of NHPs following intravenous administration of a dose of 2e13 VG/kg of AAV particles comprising the TTD-001 capsid variant. FIG. 12C is a graph showing the percentage of neuronal transduction (% HA cells among SMI311+ cells) in the thalamus, dentate nucleus, and spinal cord of the NHPs following intravenous administration of a dose of 2e13 VG/kg of AAV particles comprising the TTD-001 capsid variant.

FIG. 13 is a graph depicting the percentage of remaining SOD1 mRNA in cell lines after treatment with SOD1 targeting modulatory polynucleotide miR104-788.2 under the control of various promoters.

### DETAILED DESCRIPTION

### I. Overview

Disorders Associated with the Spinal Cord

The spinal cord is one of two components that together characterize the central nervous system (CNS; brain and spinal cord). The spinal cord connects the body to the brain, serving as a conduit for the messages and communications necessary for movement and sensation. The spinal cord is a fragile, thin, tubular bundle made up of nerve fibers and cell bodies, as well as support cells, housed within the vertebral column.

The motor neurons and pathways of the spinal cord are important for the initiation, execution, modification, and precision of movement. When these neurons and/or pathways are damaged in some manner, such as, but not limited to, trauma, tumorous growth, cardiovascular defects, inflammation, de-myelination, neuropathy, degeneration and/or cell death, the consequence is typically a defect in some form of movement. Similarly, sensory neurons and pathways of the spinal cord are critical for proprioception and sensation, and when damaged, can result in an inability to sense certain stimuli and/or pain syndromes.

Non-limiting examples of disorders such as those described above, which are associated with the spinal cord include, but are not limited to, motor neuron disease, amyotrophic lateral sclerosis (ALS; Lou Gehrig's disease), progressive bulbar palsy, pseudobulbar palsy, primary lateral sclerosis, progressive muscular atrophy, spinal muscular atrophy, post-polio syndrome, bulbar palsy, Kennedy's disease, hereditary spastic paraplegia, Friedreich's ataxia, Charcot-Marie-Tooth disease, hereditary motor and sensory neuropathy, peroneal muscular atrophy, neuropathies, de-myelinating diseases, viral de-myelination, metabolic de-myelination, multiple sclerosis, neuromyelitis optica (Devic's disease), concentric sclerosis (Bald's sclerosis), ataxias, paraplegia, spinocerebellar ataxia, acute-disseminated encephalomyelitis, complex regional pain syndrome (CPRS I and CPRS II), ataxia telangiectasia, episodic ataxia, multiple system atrophy, sporadic ataxia, lipid storage diseases, Niemann-Pick disease, Fabry disease, Faber's disease, GM1 or GM2 gangliosidoses, Tay-Sachs disease, Sandhoff disease, Krabbe disease, metachromatic leukodystrophy, Machado-Joseph disease (spinocerebellar ataxia type 3), meningitis, myelitis, myopathy, mitochondrial myopathy, encephalomyopathy, Barth syndrome, Chronic progressive external ophtalmoplegia, Kearns-Sayre syndrome, Leigh syndrome, mitochondrial DNA depletion syndromes, myoclonus epilepsy with ragged red fibers, NARP (neuropathy, ataxia and retinitis pigmentosa, diseases of the neuromuscular junction, myasthenia gravis, myoclonus, neuropathic pain, neurodegenerative diseases, Parkinson's disease, Alzheimer's disease, Huntington's disease, Lewy body disease, Vitamin B12 deficiency, subacute combined degeneration of the spinal cord (Lichtheim's disease), tropical spastic paraparesis, distal hereditary motor neuronopathies, Morvan's syndrome, leukodystrophies, and/or Rett syndrome.

In some embodiments, the compositions and methods of the present disclosure may be used to treat any disease of the central nervous system.

In some embodiments, the compositions and methods of the present disclosure may be used to treat a disease associated with the spinal cord.

In some embodiments, the compositions and methods of the present disclosure may be used for the treatment of a neurodegenerative disease.

In some embodiments, the compositions and methods of the present disclosure may be used for the treatment of a motor neuron disease.

In some embodiments, the compositions and methods of the present disclosure may be used for the treatment of amyotrophic lateral sclerosis (ALS).

### Amyotrophic Lateral Sclerosis (ALS) and SOD1

Amyotrophic lateral sclerosis (ALS), an adult-onset neurodegenerative disorder, is a progressive and fatal disease characterized by the selective death of motor neurons in the motor cortex, brainstem and spinal cord. Patients diagnosed with ALS develop a progressive muscle phenotype characterized by spasticity, hyperreflexia or hyporeflexia, fasciculations, muscle atrophy and paralysis. These motor impairments are caused by the de-innervation of muscles due to the loss of motor neurons. The major pathological features of ALS include degeneration of the corticospinal tracts and extensive loss of lower motor neurons (LMNs) or anterior horn cells (Ghatak et al., J Neuropathol Exp Neurol., 1986, 45, 385-395), degeneration and loss of Betz cells and other pyramidal cells in the primary motor cortex (Udaka et al., Acta Neuropathol, 1986, 70, 289-295; Maekawa et al., Brain, 2004, 127, 1237-1251) and reactive gliosis in the motor cortex and spinal cord (Kawamata et al., Am J Pathol., 1992, 140, 691-707; and Schiffer et al., J Neurol Sci., 1996, 139, 27-33). ALS is usually fatal within 3 to 5 years after the diagnosis due to respiratory defects and/or inflammation (Rowland L P and Shneibder N A, N Engl. J. Med., 2001, 344, 1688-1700).

A cellular hallmark of ALS is the presence of proteinaceous, ubiquitinated, cytoplasmic inclusions in degenerating motor neurons and surrounding cells (e.g., astrocytes). Ubiquitinated inclusions (i.e., Lewy body-like inclusions or Skein-like inclusions) are the most common and specific type of inclusion in ALS and are found in LMNs of the spinal cord and brainstem, and in corticospinal upper motor neurons (UMNs) (Matsumoto et al., J Neurol Sci., 1993, 115, 208-213; and Sasak and Maruyama, Acta Neuropathol., 1994, 87, 578-585). A few proteins have been identified to be components of the inclusions, including ubiquitin, Cu/Zn superoxide dismutase 1 (SOD1), peripherin and Dorfin. Neurofilamentous inclusions are often found in hyaline conglomerate inclusions (HCIs) and axonal ‘spheroids’ in spinal cord motor neurons in ALS. Other types and less specific inclusions include Bunina bodies (cystatin C-containing inclusions) and Crescent shaped inclusions (SCIs) in upper layers of the cortex. Other neuropathological features seen in ALS include fragmentation of the Golgi apparatus, mitochondrial vacuolization and ultrastructural abnormalities of synaptic terminals (Fujita et al., Acta Neuropathol. 2002, 103, 243-247).

In addition, in frontotemporal dementia ALS (FTD-ALS), cortical atrophy (including the frontal and temporal lobes) is also observed, which may cause cognitive impairment in FTD-ALS patients.

ALS is a complex and multifactorial disease and multiple mechanisms hypothesized as responsible for ALS pathogenesis include dysfunction of protein degradation, glutamate excitotoxicity, mitochondrial dysfunction, apoptosis, oxidative stress, inflammation, protein misfolding and aggregation, aberrant RNA metabolism, and altered gene expression.

About 10% of ALS cases have family history of the disease, and these patients are referred to as familial ALS (fALS) or inherited patients, commonly with a Mendelian dominant mode of inheritance and high penetrance. The remainder (approximately 90%-95%) is classified as sporadic ALS (sALS), as they are not associated with a documented family history, which is thought to be due to other risk factors, including environmental factors, genetic polymorphisms, somatic mutations, and possibly gene-environmental interactions. In most cases, familial (or inherited) ALS is inherited as autosomal dominant disease, but pedigrees with autosomal recessive and X-linked inheritance and incomplete penetrance exist. Sporadic and familial forms are clinically indistinguishable, suggesting a common pathogenesis. The precise cause of the selective death of motor neurons in ALS remains elusive. Progress in understanding the genetic factors in fALS may shed light on both forms of the disease.

Recently, an explosion in research and understanding of genetic causes of ALS has led to the discovery of mutations in more than 10 different genes now known to cause fALS. The most common ones are found in the genes encoding Cu/Zn superoxide dismutase 1 (SOD1; ˜20%) (Rosen D R et al., Nature, 1993, 362, 59-62), fused in sarcoma/translated in liposarcoma (FUS/TLS; 1-5%) and TDP-43 (TARDBP; 1-5%). Recently, a hexanucleotide repeat expansion (GGGGCC)ₙ in the C9orf72 gene was identified as the most frequent cause of fALS (˜40%) in the Western population (reviewed by Renton et al., Nat. Neurosci., 2014, 17, 17-23). Other genes mutated in ALS include alsin (ALS2), senataxin (SETX), vesicle-associated membrane protein (VAPB), angiogenin (ANG). fALS genes control different cellular mechanisms, suggesting that the pathogenesis of ALS is complicated and may be related to several different processes finally leading to motor neuron degeneration.

SOD1 is one of the three human superoxide dismutases identified and characterized in mammals: copper-zinc superoxide dismutase (Cu/ZnSOD or SOD1), manganese superoxide dismutase (MnSOD or SOD2), and extracellular superoxide dismutase (ECSOD or SOD3). SOD1 is a 32 kDa homodimer of a 153-residue polypeptide with one copper- and one zinc-binding site per subunit, which is encoded by SOD1 gene (GeneBank access No.: NM_000454.4) on human chromosome 21 (see Table 19). SOD1 catalyzes the reaction of superoxide anion (O²⁻) into molecular oxygen (O₂) and hydrogen peroxide (H₂O₂) at a bound copper ion. The intracellular concentration of SOD1 is high (ranging from 10 to 100 M), accounting for 1% of the total protein content in the central nervous system (CNS). The protein is localized not only in the cytoplasm but also in the nucleus, lysosomes, peroxisomes, and mitochondrial intermembrane spaces in eukaryotic cells (Lindenau J et al., Glia, 2000, 29, 25-34). Without wishing to be bound by theory, it is believed in some that in SOD1-ALS, mutation of superoxide dismutase 1 (SOD1) leads to the formation of insoluble SOD1 aggregates in motor neurons and to neurodegeneration.

Mutations in SOD1 gene are carried by 15-20% of fALS patients and by 1-2% of all ALS cases. Currently, at least 170 different mutations distributed throughout the 153-amino acid SOD1 polypeptide have been found to cause ALS, and an updated list can be found at the ALS online Genetic Database (ALSOD) (Wroe R et al., Amyotroph Lateral Scler., 2008, 9, 249-250). Table 1 lists some examples of mutations in SOD1 in ALS. These mutations are predominantly single amino acid substitutions (i.e. missense mutations) although deletions, insertions, and C-terminal truncations also occur. Different SOD1 mutations display different geographic distribution patterns. For instance, about half of all Americans with ALS caused by SOD1 gene mutations have a particular mutation Ala4Val (or A4V). The A4V mutation is typically associated with more severe signs and symptoms. The I113T mutation is by far the most common mutation in the United Kingdom. The most prevalent mutation in Europe is D90A substitution.

TABLE 1
Examples of SOD1 mutations in ALS

| | Mutations |
|---|---|
| Exon1 | Q22L; E21K, G; F20C; N19S; G16A, S; V14M, S; G12R; |
| (220 bp) | G10G, V, R; L8Q, V; V7E; C6G, F; V5L; A4T, V, S |
| Exon2 | T54R; E49K; H48R, Q; V47F, A; H46R; F45C; H43R; G41S, |
| (97 bp) | D; G37R; V29, insA |
| Exon3 | D76Y, V; G72S, C; L67R; P66A; N65S; S59I, S |
| (70 bp) | |
| Exon4 | D124G, V; V118L, InsAAAAC; L117V; T116T; R115G; |
| (118 bp) | G114A; I113T, F; I112M, T; G108V; L106V, F; S106L, |
| | delTCACTC; I104F; D101G, Y, H, N; E100G, K; I99V; |
| | V97L, M; D96N, V; A95T, V; G93S, V, A, C, R, D; D90V, |
| | A; A89T, V; T88delACTGCTGAC; V87A, M; N86I, S, D, |
| | K; G85R, S; L84V, F; H80R |
| Exon5 | I151T, S; I149T; V148I, G; G147D, R; C146R, stop; A145T, |
| (461 bp) | G; L144F, S; G141E, stop; A140A, G; N139D, K, H, N; |
| | G138E; T137R; S134N; E133V, delGAA, insTT; E132insTT; |
| | G127R, InsTGGG; L126S, delITT, stop; D126, delTT |

To investigate the mechanism of neuronal death associated with SOD1 gene defects, several rodent models of SOD1-linked ALS were developed in the art, which express the human SOD1 gene with different mutations, including missense mutations, small deletions or insertions. Some examples of ALS mouse models include SOD1^{G93A}, SOD1^{A4V}, SOD1^{G37R}, SOD1^{G85R}, SOD1^{D90A}, SOD1^{L84V}, SOD1^{I113T}, SOD1^{H36R/H48Q}, SOD1^{G127X}, SOD1^{L126X} and SOD1^{L126delTT}. There are two transgene rat models carrying two different human SOD1 mutations: SOD1^{H46R} and SOD1^{G93R}. These rodent ALS models can develop muscle weakness similar to human ALS patients and other pathogenic features that reflect several characteristics of the human disease, in particular, the selective death of spinal motor neurons, aggregation of protein inclusions in motor neurons and microglial activation. It is well known in the art that the transgenic rodents are good models of human SOD1-associated ALS disease and provide models for studying disease pathogenesis and developing disease treatment.

Studies in animal and cellular models showed that SOD1 pathogenic variants cause ALS by gain of function. That is to say, the superoxide dismutase enzyme gains new but harmful properties when altered by SOD1 mutations. For example, some SOD1 mutated variants in ALS increase oxidative stress (e.g., increased accumulation of toxic superoxide radicals) by disrupting redox cycle. Other studies also indicate that some SOD1 mutated variants in ALS might acquire toxic properties that are independent of its normal physiological function (such as abnormal aggregation of misfolded SOD1 variants). In the aberrant redox chemistry model, mutant SOD1 is unstable and through aberrant chemistry interacts with nonconventional substrates causing reactive oxygen species (ROS) overproduction. In the protein toxicity model, unstable, misfolded SOD1 aggregates into cytoplasmic inclusion bodies, sequestering proteins crucial for cellular processes. These two hypotheses are not mutually exclusive. It has been shown that oxidation of selected histidine residues that bind metals in the active site mediates SOD1 aggregation.

The aggregated mutant SOD1 protein may also induce mitochondrial dysfunction (Vehvilainen P et al., Front Cell Neurosci., 2014, 8, 126), impairment of axonal transport, aberrant RNA metabolism, glial cell pathology and glutamate excitotoxicity. In some sporadic ALS cases, misfolded wild-type SOD1 protein is found in diseased motor neurons which forms “toxic conformation” that is similar to familial ALS-linked SOD1 variants (Rotunno M S and Bosco D A, Front Cell Neurosci., 2013, 16, 7, 253). Such evidence suggests that ALS is a protein misfolding disease analogous to other neurodegenerative diseases such as Alzheimer's disease and Parkinson's disease.

Currently, no curative treatments are available for patients suffering from ALS. Until recently, the only FDA approved drug was Riluzole (also called Rilutek), an inhibitor of glutamate release, with a moderate effect on ALS, only extending survival by 2-3 months if it is taken for 18 months. Unfortunately, patients taking riluzole do not experience any slowing in disease progression or improvement in muscle function. Therefore, riluzole does not present a cure, or even an effective treatment. In 2017, the FDA approved Radicava (edaravone) for the treatment of ALS, the first such approval in 22 years. Administered intravenously and serving as a free-radical scavenger and anti-oxidant, Radicava has been shown to slow disease progression. In a clinical Phase 3 trial (NCT01492686) of 137 patients, Radicava slowed the decline in physical function as compared to those patients taking placebo and as determined by score on the ALS Functional Rating Scale-Revised (ALSFRS-R) (Writing group; Edaravone (MCI-186) ALS 19 Study Group Lancet Neurol. 2017 July; 16(7):505-512). The approval of Radicava is considered an advance in terms of treatment of ALS, however it is still not a cure. Researchers continue to search for better therapeutic agents.

One approach to inhibit abnormal SOD1 protein aggregation is to silence/inhibit SOD1 gene expression in ALS. It has been reported that small interfering RNAs for specific gene silencing of the mutated allele is therapeutically beneficial for the treatment of fALS (e.g., Ralgh G S et al., Nat. Medicine, 2005, 11(4), 429-433; and Raoul C et al., Nat. Medicine, 2005, 11(4), 423-428; and Maxwell M M et al., PNAS, 2004, 101(9), 3178-3183; and Ding H et al., Chinese Medical J., 2011, 124(1), 106-110; and Scharz D S et al., Plos Genet., 2006, 2(9), e140; the content of each of which is incorporated herein by reference in their entirety).

Many other RNA therapeutic agents that target SOD1 gene and modulate SOD1 expression in ALS are taught in the art, such RNA based agents include antisense oligonucleotides and double stranded small interfering RNAs. See, e.g., Wang H et al., J Biol. Chem., 2008, 283(23), 15845-15852); U.S. Pat. Nos. 7,498,316; 7,632,938; 7,678,895; 7,951,784; 7,977,314; 8,183,219; 8,309,533 and 8, 586, 554; and U.S. Patent publication Nos. 2006/0229268 and 2011/0263680; the content of each of which is herein incorporated by reference in their entirety.

The present disclosure employs viral vectors such as adeno-associated viral (AAV) vectors to deliver siRNA duplexes or SOD1 targeting polynucleotides into cells with high efficiency. The AAV vectors comprising RNAi molecules, e.g., siRNA molecules of the present disclosure may increase the delivery of active agents into motor neurons. SOD1 targeting polynucleotides may be able to inhibit SOD1 gene expression (e.g., mRNA level) significantly inside cells; therefore, ameliorating SOD1 expression induced stress inside the cells such as aggregation of protein and formation of inclusions, increased free radicals, mitochondrial dysfunction and RNA metabolism.

Such SOD1 targeting polynucleotides may be used for treating ALS. According to the present disclosure, methods for treating and/or ameliorating ALS in a patient comprises administering to the patient an effective amount of at least one SOD1 targeting polynucleotide encoding one or more siRNA duplexes into cells and allowing the inhibition/silence of SOD1 gene expression, are provided.

### Adeno-Associated Viral (AAV) Vectors

Described herein, inter alia, are compositions comprising isolated, e.g., recombinant, viral particles, e.g., AAV particles, for delivery, e.g., vectorized delivery, of a polynucleotide, e.g., a SOD1 targeting siRNA, and methods of making and using the same. Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload. The genome of the virus may be modified to contain a minimum of components for the assembly of a functional recombinant virus, or viral particle, which is loaded with or engineered to express or deliver a desired nucleic acid construct or payload, e.g., a transgene, a polynucleotide encoding a siRNA, e.g., a SOD1 targeting siRNA duplex which may be delivered to a target cell, tissue, or organism. In some embodiments, the target cell is a CNS cell. In some embodiments, the target tissue is a CNS tissue. The target CNS tissue may be brain tissue. In some embodiments, region of the CNS is a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof.

Gene therapy presents an alternative approach for ALS, and diseases sharing single-gene etiology, such as Gaucher disease and Dementia with Lewy Bodies and related disorders. AAVs are commonly used in gene therapy approaches as a result of a number of advantageous features. Without wishing to be bound by theory, it is believed in some embodiments, that expression vectors, e.g., an adeno-associated viral vector (AAVs) or AAV particle, e.g., an AAV particle described herein, can be used to administer and/or deliver a siRNA (e.g., a SOD1 targeting siRNA), in order to achieve sustained, high concentrations, allowing for longer lasting efficacy, fewer dose treatments, broad biodistribution, and/or more consistent levels of the siRNA, relative to a non-AAV therapy.

As demonstrated in some of the Examples herein below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without wishing to be being bound by theory, it is believed that these advantages may be due, in part, to the dissemination of the AAV capsid variants through the brain vasculature. In some embodiments, the AAV capsids described herein enhance the delivery of a payload, e.g., a transgene, a polynucleotide encoding a siRNA, e.g., a SOD1 targeting siRNA as described herein, to multiple regions of the brain including for example, the frontal cortex, sensory cortex, motor cortex, putamen, thalamus, cerebellar cortex, dentate nucleus, caudate, and/or hippocampus. In some embodiments, enhance the expression of a payload, e.g., a SOD1 targeting siRNA described herein, to multiple cell types in the CNS, e.g., neurons, oligodendrocytes, and/or glial cells. Without wishing to be bound by theory, an AAV particle comprising an AAV capsid polypeptide, e.g., an AAV capsid variant described herein, for the vectorized delivery of a SOD1 targeting siRNA described here can result in increased penetrance through the blood brain barrier, e.g., following intravenous administration, and/or increased biodistribution of the SOD1 targeting siRNA in the central nervous system, e.g., the brain and the spinal cord.

### II. Compositions

### Vectors

In some embodiments, the siRNA molecules described herein can be inserted into, or encoded by, vectors such as plasmids or viral vectors. Preferably, the siRNA molecules are inserted into, or encoded by, viral vectors.

Viral vectors may be Herpesvirus (HSV) vectors, retroviral vectors, adenoviral vectors, adeno-associated viral vectors, lentiviral vectors, and the like. In some specific embodiments, the viral vectors are AAV vectors.

### Adeno-Associated Viral (AAV) Vectors

AAV have a genome of about 5,000 nucleotides in length which contains two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety; SEQ ID NO: 138, Table 2, herein) VP1 refers to amino acids 1-736, VP2 refers to amino acids 138-736, and VP3 refers to amino acids 203-736. As another non-limiting example, VP1 refers to amino acids 1-743 numbered according to SEQ ID NO: 1, VP2 refers to amino acids 138-743 numbered according to SEQ ID NO: 1, and VP3 refers to amino acids 203-743 numbered according to SEQ ID NO: 1. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

The AAV vector typically requires a co-helper (e.g., adenovirus) to undergo productive infection in cells. In the absence of such helper functions, the AAV virions essentially enter host cells but do not integrate into the cells' genome.

AAV vectors have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV particle for delivery of an siRNA, e.g., an siRNA for targeting SOD1 as described herein, may be recombinant viral vectors which are replication defective as they lack sequences encoding functional Rep and Cap proteins within the viral genome. In some cases, the defective AAV genome may lack most or all coding sequences and essentially only contain one or two AAV ITR sequences and a payload sequence. In certain embodiments, the viral genome encodes a siRNA for targeting SOD1.

In some embodiments, the AAV particles of the present disclosure may be introduced into mammalian cells.

AAV particles may be modified to enhance the efficiency of delivery. Such modified AAV particles of the present disclosure can be packaged efficiently and can be used to successfully infect the target cells at high frequency and with minimal toxicity.

In other embodiments, AAV particles of the present disclosure may be used to deliver a siRNA (e.g., a siRNA targeting SOD1 as described herein) to the central nervous system (see, e.g., U.S. Pat. No. 6,180,613; the contents of which are herein incorporated by reference in their entirety) or to specific tissues of the CNS.

It is understood that the compositions described herein may have additional conservative or non-essential amino acid substitutions, which do not have a substantial effect on their functions.

### AAV Capsids and Variants Thereof

In some embodiments, an AAV particle, e.g., an AAV particle for the vectorized delivery of SOD1 targeting polynucleotide herein (e.g., a RNAi), may comprise an AAV capsid polypeptide, e.g., an AAV capsid variant.

In some embodiments, the AAV capsid polypeptide, e.g., AAV capsid variant, allows for blood brain barrier penetration following intravenous administration. In some embodiments, the AAV capsid, e.g., AAV capsid variant, allows for blood brain barrier penetration following focused ultrasound (FUS), e.g., coupled with the intravenous administration of microbubbles (FUS-MB), or MRI-guided FUS coupled with intravenous administration. In some embodiments the AAV capsid, e.g., AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid, e.g., AAV capsid variant allows for preferential transduction in a brain region relative to the transduction in the dorsal root ganglia (DRG).

In some embodiments the AAV capsid polypeptide, e.g., AAV capsid variant allows for increased distribution to a spinal cord region. In some embodiments, the spinal region comprises a cervical spinal cord region, thoracic spinal cord region, and/or lumbar spinal cord region.

In some embodiments, the AAV capsid polypeptide, e.g., an AAV capsid variant comprises a VOY101 capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 2, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequences in Table 2, or a nucleotide sequence substantially identical (e.g., having at least 70%, 75%, 80%, 85%, 90%, 92%, 95%, 97%, 98%, or 99% sequence identity) thereto.

In some embodiments, the AAV capsid variant, described herein does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, numbered relative to the amino acid sequences in Table 1 of WO2020223276, the contents of which are hereby incorporated by reference in their entirety.

In any of the embodiments described herein, a position comprising 5 consecutive amino acids corresponding to positions 586 to 599, e.g., 586 to 594, 587 to 595, 588 to 596, 589 to 597, 590 to 598 numbered relative to SEQ ID NO: 138 can be identified by providing an alignment of a reference sequence and a query sequence, wherein the reference sequence is SEQ ID NO: 138, and identifying the residues corresponding to the positions in the query sequence that correspond to positions 586 to 599, e.g., 586 to 594, 587 to 595, 588 to 596, 589 to 597, 590 to 598 in the reference sequence.

In some embodiments, the AAV capsid polypeptide, e.g., AAV capsid variant, described herein does not comprise an amino acid sequence present immediately subsequent to position 586, 588, or 589 numbered relative to SEQ ID NO: 138, at least 5 consecutive amino acids corresponding to positions 586 to 599, e.g., 586 to 594, 587 to 595, 588 to 596, 589 to 597, 590 to 598, numbered relative to SEQ ID NO: 138. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least 5 consecutive amino acids corresponding to positions 586 to 599, e.g., 586 to 594, 587 to 595, 588 to 596, 589 to 597, 590 to 598, numbered relative to SEQ ID NO: 12. In some embodiments, the AAV capsid polypeptide, e.g., AAV capsid variant, described herein, does not comprise an amino acid sequence present immediately subsequent to position 586, 588, or 589 numbered relative to SEQ ID NO: 138, having at least than 5 consecutive amino acids corresponding to positions 586 to 599, e.g., 586 to 594, 587 to 595, 588 to 596, 589 to 597, 590 to 598, numbered relative to SEQ ID NO: 13. In some embodiments, the AAV capsid polypeptide, e.g., AAV capsid variant, or the parent AAV capsid may be, at a position other than 5 consecutive amino acids corresponding to positions 586 to 599, e.g., 586 to 594, 587 to 595, 588 to 596, 589 to 597, 590 to 598, numbered relative to SEQ ID NO: 1.

TABLE 2
Exemplary full length capsid sequences

| | SEQ | |
|---|---|---|
| | ID | |
| Description | NO: | Sequence Information |
| VOY101 | 1 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9/hu.14 | 11 | MAADGYLPDWLEDNLSEGIREWWALKPGAPQPKANQQHQDNARGLVLPGYKYLGPGNGL |
| K449R | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 138 | MAADGYLPDWLEDNLSEGIREWWALKPGAPQPKANQQHQDNARGLVLPGYKYLGPGNGL |
| WT | | DKGEPVNAADAAALEHDKAYDQQLKAGDNPYLKYNHADAEFQERLKEDTSEGGNLGRAV |
| (amino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVEMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 137 | ATGGCTGCCGATGGTTATCTTCCAGATTGGCTCGAGGACAACCTTAGTGAAGGAATTCG |
| WT | | CGAGTGGTGGGCTTTGAAACCTGGAGCCCCTCAACCCAAGGCAAATCAACAACATCAAG |
| (DNA) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| PHP.B | 12 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| PHP.N | 13 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAVrh10 | 14 | MAADGYLPDWLEDNLSEGIREWWDLKPGAPKPKANQQKQDDGRGLVLPGYKYLGPFNGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEPSPQRSPDSSTGIGKKGQQPAKKRLNFGQT |
| | | GDSESVPDPQPIGEPPAGPSGLGSGTMAAGGGAPMADNNEGADGVGSSSGNWHCDSTWL |
| | | GDRVITTSTRTWALPTYNNHLYKQISNGTSGGSTNDNTYFGYSTPWGYFDFNRFHCHFS |
| | | PRDWQRLINNNWGFRPKRLNFKLFNIQVKEVTQNEGTKTIANNLTSTIQVFTDSEYQLP |
| | | YVLGSAHQGCLPPFPADVFMIPQYGYLTLNNGSQAVGRSSFYCLEYFPSQMLRTGNNFE |
| | | FSYQFEDVPFHSSYAHSQSLDRLMNPLIDQYLYYLSRTQSTGGTAGTQQLLFSQAGPNN |
| | | MSAQAKNWLPGPCYRQQRVSTTLSQNNNSNFAWTGATKYHLNGRDSLVNPGVAMATHKD |
| | | DEERFFPSSGVLMFGKQGAGKDNVDYSSVMLTSEEEIKTTNPVATEQYGVVADNLQQQN |
| | | AAPIVGAVNSQGALPGMVWQNRDVYLQGPIWAKIPHTDGNFHPSPLMGGFGLKHPPPQI |
| | | LIKNTPVPADPPTTFSQAKLASFITQYSTGQVSVEIEWELQKENSKRWNPEIQYTSNYY |
| | | KSTNVDFAVNTDGTYSEPRPIGTRYLTRNL |
| | | ATGGCTGCCGATGGTTATCTTCCAGATTGGCTCGAGGACAACCTCTCTGAGGGCATTCG |
| | | CGAGTGGTGGGACTTGAAACCTGGAGCCCCGAAACCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAGAGACCGGTAGAGCCATCACCCCAGCGTTCTCCAGACTCCTCTA |
| | | CGGGCATCGGCAAGAAAGGCCAGCAGCCCGCGAAAAAGAGACTCAACTTTGGGCAGACT |
| | | GGCGACTCAGAGTCAGTGCCCGACCCTCAACCAATCGGAGAACCCCCCGCAGGCCCCTC |
| | | TGGTCTGGGATCTGGTACAATGGCTGCAGGCGGTGGCGCTCCAATGGCAGACAATAACG |
| | | AAGGCGCCGACGGAGTGGGTAGTTCCTCAGGAAATTGGCATTGCGATTCCACATGGCTG |
| | | GGCGACAGAGTCATCACCACCAGCACCCGAACCTGGGCCCTCCCCACCTACAACAACCA |
| | | CCTCTACAAGCAAATCTCCAACGGGACTTCGGGAGGAAGCACCAACGACAACACCTACT |
| AAVrh10 | 15 | TCGGCTACAGCACCCCCTGGGGGTATTTTGACTTTAACAGATTCCACTGCCACTTCTCA |
| (DNA) | | CCACGTGACTGGCAGCGACTCATCAACAACAACTGGGGATTCCGGCCCAAGAGACTCAA |
| | | CTTCAAGCTCTTCAACATCCAGGTCAAGGAGGTCACGCAGAATGAAGGCACCAAGACCA |
| | | TCGCCAATAACCTTACCAGCACGATTCAGGTCTTTACGGACTCGGAATACCAGCTCCCG |
| | | TACGTCCTCGGCTCTGCGCACCAGGGCTGCCTGCCTCCGTTCCCGGCGGACGTCTTCAT |
| | | GATTCCTCAGTACGGGTACCTGACTCTGAACAATGGCAGTCAGGCCGTGGGCCGTTCCT |
| | | CCTTCTACTGCCTGGAGTACTTTCCTTCTCAAATGCTGAGAACGGGCAACAACTTTGAG |
| | | TTCAGCTACCAGTTTGAGGACGTGCCTTTTCACAGCAGCTACGCGCACAGCCAAAGCCT |
| | | GGACCGGCTGATGAACCCCCTCATCGACCAGTACCTGTACTACCTGTCTCGGACTCAGT |
| | | CCACGGGAGGTACCGCAGGAACTCAGCAGTTGCTATTTTCTCAGGCCGGGCCTAATAAC |
| | | ATGTCGGCTCAGGCCAAAAACTGGCTACCCGGGCCCTGCTACCGGCAGCAACGCGTCTC |
| | | CACGACACTGTCGCAAAATAACAACAGCAACTTTGCCTGGACCGGTGCCACCAAGTATC |
| | | ATCTGAATGGCAGAGACTCTCTGGTAAATCCCGGTGTCGCTATGGCAACCCACAAGGAC |
| | | GACGAAGAGCGATTTTTTCCGTCCAGCGGAGTCTTAATGTTTGGGAAACAGGGAGCTGG |
| | | AAAAGACAACGTGGACTATAGCAGCGTTATGCTAACCAGTGAGGAAGAAATTAAAACCA |
| | | CCAACCCAGTGGCCACAGAACAGTACGGCGTGGTGGCCGATAACCTGCAACAGCAAAAC |
| | | GCCGCTCCTATTGTAGGGGCCGTCAACAGTCAAGGAGCCTTACCTGGCATGGTCTGGCA |
| | | GAACCGGGACGTGTACCTGCAGGGTCCTATCTGGGCCAAGATTCCTCACACGGACGGAA |
| | | ACTTTCATCCCTCGCCGCTGATGGGAGGCTTTGGACTGAAACACCCGCCTCCTCAGATC |
| | | CTGATTAAGAATACACCTGTTCCCGCGGATCCTCCAACTACCTTCAGTCAAGCTAAGCT |
| | | GGCGTCGTTCATCACGCAGTACAGCACCGGACAGGTCAGCGTGGAAATTGAATGGGAGC |
| | | TGCAGAAAGAAAACAGCAAACGCTGGAACCCAGAGATTCAATACACTTCCAACTACTAC |
| | | AAATCTACAAATGTGGACTTTGCTGTTAACACAGATGGCACTTATTCTGAGCCTCGCCC |
| | | CATCGGCACCCGTTACCTCACCCGTAATCTGTAA |
| AAV2 | 16 | MAADGYLPDWLEDTLSEGIRQWWKLKPGPPPPKPAERHKDDSRGLVLPGYKYLGPFNGL |
| (amino acid) | | DKGEPVNEADAAALEHDKAYDRQLDSGDNPYLKYNHADAEFQERLKEDTSFGGNLGRAV |
| | | FQAKKRVLEPLGLVEEPVKTAPGKKRPVEHSPVEPDSSSGTGKAGQQPARKRLNFGQTG |
| | | DADSVPDPQPLGQPPAAPSGLGTNTMATGSGAPMADNNEGADGVGNSSGNWHCDSTWMG |
| | | DRVITTSTRTWALPTYNNHLYKQISSQSGASNDNHYFGYSTPWGYFDFNRFHCHFSPRD |
| | | WQRLINNNWGFRPKRLNFKLFNIQVKEVTQNDGTTTIANNLTSTVQVFTDSEYQLPYVL |
| | | GSAHQGCLPPFPADVFMVPQYGYLTLNNGSQAVGRSSFYCLEYFPSQMLRTGNNFTFSY |
| | | TFEDVPFHSSYAHSQSLDRLMNPLIDQYLYYLSRTNTPSGTTTQSRLQFSQAGASDIRD |
| | | QSRNWLPGPCYRQQRVSKTSADNNNSEYSWTGATKYHLNGRDSLVNPGPAMASHKDDEE |
| | | KFFPQSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPPPQILIK |
| | | NTPVPANPSTTFSAAKFASFITQYSTGQVSVEIEWELQKENSKRWNPEIQYTSNYNKSV |
| | | NVDFTVDINGVYSEPRPIGTRYLTRNL |
| AAV2 | 17 | ATGGCTGCCGATGGTTATCTTCCAGATTGGCTCGAGGACACTCTCTCTGAAGGAATAAG |
| (DNA) | | ACAGTGGTGGAAGCTCAAACCTGGCCCACCACCACCAAAGCCCGCAGAGCGGCATAAGG |
| | | ACGACAGCAGGGGTCTTGTGCTTCCTGGGTACAAGTACCTCGGACCCTTCAACGGACTC |
| | | GACAAGGGAGAGCCGGTCAACGAGGCAGACGCCGCGGCCCTCGAGCACGACAAAGCCTA |
| | | CGACCGGCAGCTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGACGAGCAGTC |
| | | TTCCAGGCGAAAAAGAGGGTTCTTGAACCTCTGGGCCTGGTTGAGGAACCTGTTAAGAC |
| | | GGCTCCGGGAAAAAAGAGGCCGGTAGAGCACTCTCCTGTGGAGCCAGACTCCTCCTCGG |
| | | GAACCGGAAAGGCGGGCCAGCAGCCTGCAAGAAAAAGATTGAATTTTGGTCAGACTGGA |
| | | GACGCAGACTCAGTACCTGACCCCCAGCCTCTCGGACAGCCACCAGCAGCCCCCTCTGG |
| | | TCTGGGAACTAATACGATGGCTACAGGCAGTGGCGCACCAATGGCAGACAATAACGAGG |
| | | GCGCCGACGGAGTGGGTAATTCCTCGGGAAATTGGCATTGCGATTCCACATGGATGGGC |
| | | GACAGAGTCATCACCACCAGCACCCGAACCTGGGCCCTGCCCACCTACAACAACCACCT |
| | | CTACAAACAAATTTCCAGCCAATCAGGAGCCTCGAACGACAATCACTACTTTGGCTACA |
| | | GCACCCCTTGGGGGTATTTTGACTTCAACAGATTCCACTGCCACTTTTCACCACGTGAC |
| | | TGGCAAAGACTCATCAACAACAACTGGGGATTCCGACCCAAGAGACTCAACTTCAAGCT |
| | | CTTTAACATTCAAGTCAAAGAGGTCACGCAGAATGACGGTACGACGACGATTGCCAATA |
| | | ACCTTACCAGCACGGTTCAGGTGTTTACTGACTCGGAGTACCAGCTCCCGTACGTCCTC |
| | | GGCTCGGCGCATCAAGGATGCCTCCCGCCGTTCCCAGCAGACGTCTTCATGGTGCCACA |
| | | GTATGGATACCTCACCCTGAACAACGGGAGTCAGGCAGTAGGACGCTCTTCATTTTACT |
| | | GCCTGGAGTACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCTGGACCGTCT |
| | | CATGAATCCTCTCATCGACCAGTACCTGTATTACTTGAGCAGAACAAACACTCCAAGTG |
| | | GAACCACCACGCAGTCAAGGCTTCAGTTTTCTCAGGCCGGAGCGAGTGACATTCGGGAC |
| | | CAGTCTAGGAACTGGCTTCCTGGACCCTGTTACCGCCAGCAGCGAGTATCAAAGACATC |
| | | TGCGGATAACAACAACAGTGAATACTCGTGGACTGGAGCTACCAAGTACCACCTCAATG |
| | | GCAGAGACTCTCTGGTGAATCCGGGCCCGGCCATGGCAAGCCACAAGGACGATGAAGAA |
| | | AAGTTTTTTCCTCAGAGCGGGGTTCTCATCTTTGGGAAGCAAGGCTCAGAGAAAACAAA |
| | | TGTGGACATTGAAAAGGTCATGATTACAGACGAAGAGGAAATCAGGACAACCAATCCCG |
| | | TGGCTACGGAGCAGTATGGTTCTGTATCTACCAACCTCCAGAGAGGCAACAGACAAGCA |
| | | GCTACCGCAGATGTCAACACACAAGGCGTTCTTCCAGGCATGGTCTGGCAGGACAGAGA |
| | | TGTGTACCTTCAGGGGCCCATCTGGGCAAAGATTCCACACACGGACGGACATTTTCACC |
| | | CCTCTCCCCTCATGGGTGGATTCGGACTTAAACACCCTCCTCCACAGATTCTCATCAAG |
| | | AACACCCCGGTACCTGCGAATCCTTCGACCACCTTCAGTGCGGCAAAGTTTGCTTCCTT |
| | | CATCACACAGTACTCCACGGGACAGGTCAGCGTGGAGATCGAGTGGGAGCTGCAGAAGG |
| | | AAAACAGCAAACGCTGGAATCCCGAAATTCAGTACACTTCCAACTACAACAAGTCTGTT |
| | | AATGTGGACTTTACTGTGGACACTAATGGCGTGTATTCAGAGCCTCGCCCCATTGGCAC |
| | | CAGATACCTGACTCGTAATCTGTAA |
| AAV1 | 18 | MAADGYLPDWLEDNLSEGIREWWDLKPGAPKPKANQQKQDDGRGLVLPGYKYLGPFNGL |
| (amino acid) | | DKGEPVNAADAAALEHDKAYDQQLKAGDNPYLRYNHADAEFQERLQEDTSEGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAV1 | 19 | atggctgccgatggttatcttccagattggctcgaggacaacctctctgagggcattcg |
| (DNA) | | cgagtggtgggacttgaaacctggagccccgaagcccaaagccaaccagcaaaagcagg |
| | | acgacggccggggtctggtgcttcctggctacaagtacctcggacccttcaacggactc |
| | | gacaagggggagcccgtcaacgcggcggacgcagcggccctcgagcacgacaaggccta |
| | | cgaccagcagctcaaagcgggtgacaatccgtacctgcggtataaccacgccgacgccg |
| | | agtttcaggagcgtctgcaagaagatacgtcttttgggggcaacctcgggcgagcagtc |
| | | ttccaggccaagaagcgggttctcgaacctctcggtctggttgaggaaggcgctaagac |
| | | ggctcctggaaagaaacgtccggtagagcagtcgccacaagagccagactcctcctcgg |
| | | gcatcggcaagacaggccagcagcccgctaaaaagagactcaattttggtcagactggc |
| | | gactcagagtcagtccccgatccacaacctctcggagaacctccagcaacccccgctgc |
| | | tgtgggacctactacaatggcttcaggcggtggcgcaccaatggcagacaataacgaag |
| | | gcgccgacggagtgggtaatgcctcaggaaattggcattgcgattccacatggctgggc |
| | | gacagagtcatcaccaccagcacccgcacctgggccttgcccacctacaataaccacct |
| | | ctacaagcaaatctccagtgcttcaacgggggccagcaacgacaaccactacttcggct |
| | | acagcaccccctgggggtattttgatttcaacagattccactgccacttttcaccacgt |
| | | gactggcagcgactcatcaacaacaattggggattccggcccaagagactcaacttcaa |
| | | actcttcaacatccaagtcaaggaggtcacgacgaatgatggcgtcacaaccatcgcta |
| | | ataaccttaccagcacggttcaagtcttctcggactcggagtaccagcttccgtacgtc |
| | | ctcggctctgcgcaccagggctgcctccctccgttcccggcggacgtgttcatgattcc |
| | | gcaatacggctacctgacgctcaacaatggcagccaagccgtgggacgttcatcctttt |
| | | actgcctggaatatttcccttctcagatgctgagaacgggcaacaactttaccttcagc |
| | | tacacctttgaggaagtgcctttccacagcagctacgcgcacagccagagcctggaccg |
| | | gctgatgaatcctctcatcgaccaatacctgtattacctgaacagaactcaaaatcagt |
| | | ccggaagtgcccaaaacaaggacttgctgtttagccgtgggtctccagctggcatgtct |
| | | gttcagcccaaaaactggctacctggaccctgttatcggcagcagcgcgtttctaaaac |
| | | aaaaacagacaacaacaacagcaattttacctggactggtgcttcaaaatataacctca |
| | | atgggcgtgaatccatcatcaaccctggcactgctatggcctcacacaaagacgacgaa |
| | | gacaagttctttcccatgagcggtgtcatgatttttggaaaagagagcgccggagcttc |
| | | aaacactgcattggacaatgtcatgattacagacgaagaggaaattaaagccactaacc |
| | | ctgtggccaccgaaagatttgggaccgtggcagtcaatttccagagcagcagcacagac |
| | | cctgcgaccggagatgtgcatgctatgggagcattacctggcatggtgtggcaagatag |
| | | agacgtgtacctgcagggtcccatttgggccaaaattcctcacacagatggacactttc |
| | | acccgtctcctcttatgggcggctttggactcaagaacccgcctcctcagatcctcatc |
| | | aaaaacacgcctgttcctgcgaatcctccggcggagttttcagctacaaagtttgcttc |
| | | attcatcacccaatactccacaggacaagtgagtgtggaaattgaatgggagctgcaga |
| | | aagaaaacagcaagcgctggaatcccgaagtgcagtacacatccaattatgcaaaatct |
| | | gccaacgttgattttactgtggacaacaatggactttatactgagcctcgccccattgg |
| | | cacccgttaccttacccgtcccctgtaa |
| AAV5 | 20 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMTNNLQGSNTYALENTMIFNS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |
| AAV5 | 21 | atgtcttttgttgatcaccctccagattggttggaagaagttggtgaaggtcttcgcga |
| (DNA) | | gtttttgggccttgaagcgggcccaccgaaaccaaaacccaatcagcagcatcaagatc |
| | | aagcccgtggtcttgtgctgcctggttataactatctcggacccggaaacggtctcgat |
| | | cgaggagagcctgtcaacagggcagacgaggtcgcgcgagagcacgacatctcgtacaa |
| | | cgagcagcttgaggcgggagacaacccctacctcaagtacaaccacgcggacgccgagt |
| | | ttcaggagaagctcgccgacgacacatccttcgggggaaacctcggaaaggcagtcttt |
| | | caggccaagaaaagggttctcgaaccttttggcctggttgaagagggtgctaagacggc |
| | | ccctaccggaaagcggatagacgaccactttccaaaaagaaagaaggcccggaccgaag |
| | | aggactccaagccttccacctcgtcagacgccgaagctggacccagcggatcccagcag |
| | | ctgcaaatcccagcccaaccagcctcaagtttgggagctgatacaatgtctgcgggagg |
| | | tggcggcccattgggcgacaataaccaaggtgccgatggagtgggcaatgcctcgggag |
| | | attggcattgcgattccacgtggatgggggacagagtcgtcaccaagtccacccgaacc |
| | | tgggtgctgcccagctacaacaaccaccagtaccgagagatcaaaagcggctccgtcga |
| | | cggaagcaacgccaacgcctactttggatacagcaccccctgggggtactttgacttta |
| | | accgcttccacagccactggagcccccgagactggcaaagactcatcaacaactactgg |
| | | ggcttcagaccccggtccctcagagtcaaaatcttcaacattcaagtcaaagaggtcac |
| | | ggtgcaggactccaccaccaccatcgccaacaacctcacctccaccgtccaagtgttta |
| | | cggacgacgactaccagctgccctacgtcgtcggcaacgggaccgagggatgcctgccg |
| | | gccttccctccgcaggtctttacgctgccgcagtacggttacgcgacgctgaaccgcga |
| | | caacacagaaaatcccaccgagaggagcagcttcttctgcctagagtactttcccagca |
| | | agatgctgagaacgggcaacaactttgagtttacctacaactttgaggaggtgcccttc |
| | | cactccagcttcgctcccagtcagaacctcttcaagctggccaacccgctggtggacca |
| | | gtacttgtaccgcttcgtgagcacaaataacactggcggagtccagttcaacaagaacc |
| | | tggccgggagatacgccaacacctacaaaaactggttcccggggcccatgggccgaacc |
| | | cagggctggaacctgggctccggggtcaaccgcgccagtgtcagcgccttcgccacgac |
| | | caataggatggagctcgagggcgcgagttaccaggtgcccccgcagccgaacggcatga |
| | | ccaacaacctccagggcagcaacacctatgccctggagaacactatgatcttcaacagc |
| | | cagccggcgaacccgggcaccaccgccacgtacctcgagggcaacatgctcatcaccag |
| | | cgagagcgagacgcagccggtgaaccgcgtggcgtacaacgtcggcgggcagatggcca |
| | | ccaacaaccagagctccaccactgcccccgcgaccggcacgtacaacctccaggaaatc |
| | | gtgcccggcagcgtgtggatggagagggacgtgtacctccaaggacccatctgggccaa |
| | | gatcccagagacgggggcgcactttcacccctctccggccatgggggattcggactca |
| | | aacacccaccgcccatgatgctcatcaagaacacgcctgtgcccggaaatatcaccagc |
| | | ttctcggacgtgcccgtcagcagcttcatcacccagtacagcaccgggcaggtcaccgt |
| | | ggagatggagtgggagctcaagaaggaaaactccaagaggtggaacccagagatccagt |
| | | acacaaacaactacaacgacccccagtttgtggactttgccccggacagcaccggggaa |
| | | tacagaaccaccagacctatcggaacccgataccttacccgacccctttaa |

In some embodiments, the AAV capsid polypeptide, e.g., the AAV capsid variant, comprises the amino acid sequence of any of SEQ ID NOs: 1, 11, 138, 12, 13, 14, 16, 18, 20, or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of any of SEQ ID NOs: 1, 11, 138, 12, 13, 14, 16, 18, 20. In some embodiments, the AAV capsid polypeptide, e.g., the AAV capsid variant, comprises an amino acid sequence encoded by the nucleotide sequence of any of SEQ ID NOs: 137, 15, 17, 19, 21, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding the AAV capsid polypeptide, e.g., the AAV capsid variant, comprises the nucleotide sequence of any of SEQ ID NOs: 137, 15, 17, 19, or 21, or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises a nucleotide sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the nucleotide sequence of any of SEQ ID NOs: 137, 15, 17, 19, or 21.

In some embodiments, the AAV capsid polypeptide, e.g., the AAV capsid variant, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 138. In some embodiments, the AAV capsid polypeptide, e.g., the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide, e.g., the AAV capsid variant, comprises substitution at position K449, e.g., a K449R substitution, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262). In some embodiments, the peptide is present immediately subsequent to position 588, relative to a reference sequence numbered according to SEQ ID NO: 138. In some embodiments, the capsid polypeptide comprises the amino acid substitutions of A587D and Q588G, numbered according to SEQ ID NO: 138.

In some embodiments, the AAV capsid polypeptide, e.g., the AAV capsid variant, comprises the amino acid substitution of K449R, numbered according to SEQ ID NO: 138; and a peptide comprising the amino acid sequence of TLAVPFK (SEQ ID NO: 1262), wherein the peptide is present immediately subsequent to position 588, relative to a reference sequence numbered according to SEQ ID NO: 138.

In some embodiments, the AAV capsid polypeptide, e.g., the AAV capsid variant, comprises the amino acid substitution of K449R, numbered according to SEQ ID NO: 138; an peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 11, optionally wherein position 449 is not R.

In some embodiments, the capsid polypeptide, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 1.

In some embodiments, an AAV capsid variant disclosed herein comprises a modification in loop VIII of AAV9, e.g., at positions between 580-599, e.g., at positions 587, 588, 589, and/or 590, numbered relative to SEQ ID NO: 5, 8, 138 or 3636-3647. In some embodiments, loop (e.g., loop VIII) is used interchangeably herein with the term variable region (e.g., variable region VIII), or VR (e.g., VR-VIII). In some embodiments loop VIII comprises positions 580-599 (e.g., amino acids VATNHQSAQAQAQTGWVQNQ (SEQ ID NO: 1195), numbered according to SEQ ID NO: 138. In some embodiments, loop VIII comprises positions 582-593 (e.g., amino acids TNHQSAQAQAQT (SEQ ID NO: 1196), numbered according to SEQ ID NO: 138. In some embodiments loop VIII comprises positions 587-593 (e.g., amino acids AQAQAQT (SEQ ID NO: 1197), numbered according to SEQ ID NO: 138. In some embodiments loop VIII comprises positions 587-590 (e.g., amino acids AQAQ, SEQ ID NO: 3759), numbered according to SEQ ID NO: 138. In some embodiments, loop VIII or variable region VIII (VR-VIII) is as described in DiMattia et al. “Structural Insights into the Unique Properties of the Adeno-Associated Virus Serotype 9,” Journal of Virology, 12(86):6947-6958 (the contents of which are hereby incorporated by reference in their entirety), e.g., comprising positions 581-593 (e.g., ATNHQSAQAQAQT (SEQ ID NO: 1198), numbered according to SEQ ID NO: 138.

In some embodiments, an AAV particle described herein comprises an AAV capsid polypeptide, e.g., an AAV capsid variant. In some embodiments, the AAV capsid polypeptide, e.g., the AAV capsid variant, comprises a peptide sequence as described in Table 3, e.g., any one of peptides 1, 2, 3, 4, 5, 6, 7, 8, 9, 10, 11, or 12. In some embodiments, the peptide may increase distribution of an AAV particle to a cell, region, or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei. In some embodiments, the peptide may increase biodistribution of an AAV particle to a cell, region, or tissue of the PNS. The cell or tissue of the PNS may be, but is not limited to, a dorsal root ganglion (DRG). In some embodiments, the peptide may increase biodistribution of an AAV particle to the CNS (e.g., the cortex) after intravenous administration. In some embodiments, the peptide may increase biodistribution of an AAV particle to the CNS (e.g., the cortex) following focused ultrasound (FUS), e.g., coupled with the intravenous administration of microbubbles (FUS-MB), or MRI-guided FUS coupled with intravenous administration. In some embodiments, the peptide may increase biodistribution of an AAV particle to the PNS (e.g., DRG) after intravenous administration. In some embodiments, the peptide may increase biodistribution of an AAV particle to the PNS (e.g., DRG) following focused ultrasound (FUS), e.g., coupled with the intravenous administration of microbubbles (FUS-MB), or MRI-guided FUS coupled with intravenous administration. In some embodiments, the peptide may increase biodistribution of an AAV particle to a cell, region, or tissue of a muscle. In some embodiments, the muscle is a heart muscle. In some embodiments, the peptide may direct an AAV particle to a muscle cell, region, or tissue after intravenous administration.

TABLE 3
Exemplary Peptide Sequences

| | SEQ | Amino | SEQ | |
|---|---|---|---|---|
| Pep- | ID | Acid | ID | Nucleotide |
| tide | NO: | Sequence | NO: | Sequence |
| 1 | 3648 | PLNGAVHLY | 3660 | ccgcttaatggtgcc |
| | | | | gtccatctttat |
| 2 | 3649 | RDSPKGW | 3661 | cgtgattctccgaag |
| | | | | ggttggca |
| 3 | 3650 | YSTDVRM | 3662 | tattctacggatgtg |
| | | | | aggatgca |
| 4 | 3651 | IVMNSLK | 3663 | attgttatgaattcg |
| | | | | ttgaaggc |
| 5 | 3652 | RESPRGL | 3664 | cgggagagtcctcgt |
| | | | | gggctgca |
| 6 | 3653 | SFNDTRA | 3665 | agttttaatgatact |
| | | | | agggctca |
| 7 | 3654 | GGTLAVVSL | 3666 | ggtggtacgttggcc |
| | | | | gtcgtgtcgctt |
| 8 | 3655 | YGLPKGP | 3667 | tatgggttgccgaag |
| | | | | ggtcct |
| 9 | 3656 | STGTLRL | 3668 | tcgactgggacgctt |
| | | | | cggctt |
| 10 | 3657 | YSTDERM | 3669 | tattcgacggatgag |
| | | | | aggatg |
| 11 | 3658 | YSTDERK | 3670 | tattcgacggatgag |
| | | | | aggaag |
| 12 | 3659 | YVSSVKM | 3671 | tatgtttcgtctgtt |
| | | | | aagatg |

In some embodiments, the AAV capsid polypeptide, e.g., the AAV capsid variant, comprises at least 3, 4, 5, 6, 7, 8, or 9 consecutive amino acids from the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises an amino acid sequence comprising at least one, two, or three but no more than four modifications, e.g., substitutions (e.g., conservative substitutions), relative to the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises an amino acid sequence comprising at least one, two, or three but no more than four modifications, e.g., substitutions, (e.g., conservative substitutions), insertions, or deletions, relative to any of the amino acid sequence of Table 20. In some embodiments, the AAV capsid polypeptide, e.g., the AAV capsid variant, comprises an amino acid sequence comprising at least one, two, or three but no more than four different amino acids, relative to any of the amino acid sequence of Table 20. In some embodiments, the AAV capsid variant comprises an amino acid sequence comprising at least one, two, or three but no more than four modifications, e.g., substitutions, (e.g., conservative substitutions), insertions, or deletions, relative to the amino acid sequence of any of SEQ ID NO: 3648-3659. In some embodiments, the AAV capsid variant comprises an amino acid sequence comprising at least one, two, or three but no more than four different amino acids relative to the amino acid sequence of any of SEQ ID NO: 3648-3659. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid polypeptide, e.g., the AAV capsid variant, comprises at least 3, 4, 5, 6, 7, 8, or 9 consecutive amino acids from the amino acid sequence of any of SEQ ID NO: 3648-3659. In some embodiments, the 3 consecutive amino acids comprise PLN. In some embodiments, the 4 consecutive amino acids comprise PLNG (SEQ ID NO: 3678). In some embodiments, the 5 consecutive amino acids comprise PLNGA (SEQ ID NO: 3679). In some embodiments, the 6 consecutive amino acids comprise PLNGAV (SEQ ID NO: 3680). In some embodiments, the 7 consecutive amino acids comprise PLNGAVH (SEQ ID NO: 3681). In some embodiments, the 8 consecutive amino acids comprise PLNGAVHL (SEQ ID NO: 3682). In some embodiments, the 9 consecutive amino acids comprise PLNGAVHLY (SEQ ID NO: 3648).

In some embodiments, the four consecutive amino acids comprise NGAV (SEQ ID NO: 3683). In some embodiments, the four consecutive amino acids comprise GAVH (SEQ ID NO: 3684). In some embodiments, the five consecutive amino acids comprise NGAVH (SEQ ID NO: 3685). In some embodiments, the five consecutive amino acids comprise GAVHL (SEQ ID NO: 3686). In some embodiments, the five consecutive amino acids comprise AVHLY (SEQ ID NO: 3687). In some embodiments, the six consecutive amino acids comprise NGAVHL (SEQ ID NO: 3688). In some embodiments, the seven consecutive amino acids comprise NGAVHLY (SEQ ID NO: 3689).

In some embodiments, the 3 consecutive amino acids comprise YST. In some embodiments, the 4 consecutive amino acids comprise YSTD (SEQ ID NO: 3690). In some embodiments, the 5 consecutive amino acids comprise YSTDE (SEQ ID NO: 3691). In some embodiments, the 5 consecutive amino acids comprise YSTDV (SEQ ID NO: 3700). In some embodiments, the 6 consecutive amino acids comprise YSTDER (SEQ ID NO: 3692). In some embodiments, the 6 consecutive amino acids comprise YSTDVR (SEQ ID NO: 3701). In some embodiments, the 7 consecutive amino acids comprise YSTDERM (SEQ ID NO: 3657). In some embodiments, the 7 consecutive amino acids comprise YSTDERK (SEQ ID NO: 3658). In some embodiments, the 7 consecutive amino acids comprise YSTDVRM (SEQ ID NO: 3650).

In some embodiments, the 3 consecutive amino acids comprise IVM. In some embodiments, the 4 consecutive amino acids comprise IVMN (SEQ ID NO: 3693). In some embodiments, the 5 consecutive amino acids comprise IVMNS (SEQ ID NO: 3694). In some embodiments, the 6 consecutive amino acids comprise IVMNSL (SEQ ID NO: 3695). In some embodiments, the 7 consecutive amino acids comprise IVMNSLK (SEQ ID NO: 3651).

In some embodiments, the AAV capsid polypeptide, e.g., the AAV capsid variant, comprises an amino acid sequence comprising at least one, two, or three but no more than four modifications, e.g., substitutions (e.g., conservative substitutions), relative to the amino acid sequence of any of SEQ ID NO: 3648-3659. In some embodiments, the modification is a conservative substitution.

In some embodiments, the AAV capsid polypeptide, e.g., the AAV capsid variant, comprises the amino acid sequence of PLNGAVHLY (SEQ ID NO: 3648), an amino acid sequence comprising at least one, two, or three but no more than four modifications, e.g., substitutions (e.g., conservative substitutions), insertions, or deletions, relative to the amino acid sequence of PLNGAVHLY (SEQ ID NO: 3648), or an amino acid sequence comprising at least one, two, or three but no more than four different amino acids relative to the amino acid sequence of PLNGAVHLY (SEQ ID NO: 3648), optionally wherein position 7 is H.

In some embodiments, the AAV capsid polypeptide, e.g., the AAV capsid variant, comprises the amino acid sequence of RDSPKGW (SEQ ID NO: 3649), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of RDSPKGW (SEQ ID NO: 3649), or an amino acid sequence comprising at least one, two, or three but no more than four different amino acids relative to the amino acid sequence of RDSPKGW (SEQ ID NO: 3649).

In some embodiments, the AAV capsid polypeptide, e.g., the AAV capsid variant, comprises the amino acid sequence of IVMNSLK (SEQ ID NO: 3651), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of IVMNSLK (SEQ ID NO: 3651), or an amino acid sequence comprising at least one, two, or three but no more than four different amino acids relative to the amino acid sequence of IVMNSLK (SEQ ID NO: 3651).

In some embodiments, the AAV capsid polypeptide, e.g., the AAV capsid variant, comprises the amino acid sequence of YSTDVRM (SEQ ID NO: 3650), an amino acid sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of YSTDVRM (SEQ ID NO: 3650), or an amino acid sequence comprising at least one, two, or three but no more than four different amino acids relative to the amino acid sequence of YSTDVRM (SEQ ID NO: 3650).

In some embodiments, the AAV capsid polypeptide, e.g., the AAV capsid variant, comprises the amino acid sequence of RESPRGL (SEQ ID NO: 3652), a sequence comprising at least one, two, or three modifications, e.g., substitutions (e.g., conservative substitutions), insertions, or deletions, but no more than four modifications, e.g., substitutions (e.g., conservative substitutions), insertions, or deletions, relative to the amino acid sequence of RESPRGL (SEQ ID NO: 3652), or an amino acid sequence comprising at least one, two, or three but no more than four different amino acids relative to the amino acid sequence of RESPRGL (SEQ ID NO: 3652).

In some embodiments, the AAV capsid variant comprises the amino acid sequence of any of SEQ ID NO: 3648-3659. In some embodiments, the amino acid sequence is present in loop VIII of an AAV capsid variant described herein. In some embodiments, the AAV capsid variant comprises the amino acid sequence of SEQ ID NO: 3648. In some embodiments, the AAV capsid variant comprises the amino acid sequence of SEQ ID NO: 3649. In some embodiments, the AAV capsid variant comprises the amino acid sequence of SEQ ID NO: 3651. In some embodiments, the amino acid sequence is present immediately subsequent to position 586, relative to a reference sequence numbered according to the amino acid sequence of SEQ ID NO: 138. In some embodiments, the amino acid sequence is present immediately subsequent to position 588, relative to a reference sequence numbered according to the amino acid sequence of SEQ ID NO: 138. In some embodiments, the amino acid sequence is present immediately subsequent to position 589, relative to a reference sequence numbered according to the amino acid sequence of SEQ ID NO: 138.

In some embodiments, the AAV capsid polypeptide, e.g., the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of any of SEQ ID NOs: 3660-3671.

In some embodiments, the AAV capsid polypeptide, e.g., the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of any of SEQ ID NOs: 3660-3671. In some embodiments, the AAV capsid polypeptide, e.g., the AAV capsid variant (e.g., an AAV capsid variant described herein), comprises an amino acid sequence encoded by a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, nucleic acid sequence encoding the AAV capsid variant, e.g., an AAV capsid variant described herein, comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to any of SEQ ID NOs: 3660-3671. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of any of SEQ ID NOs: 3660-3671.

In some embodiments, the nucleotide sequence encoding the AAV capsid polypeptide, e.g., the AAV capsid variant (e.g., an AAV capsid variant described herein), comprises the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 3660. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3660.

In some embodiments, the nucleotide sequence encoding the AAV capsid polypeptide, e.g., the AAV capsid variant (e.g., an AAV capsid variant described herein), comprises the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, of the nucleotide sequences relative to SEQ ID NO: 3663. In some embodiments, the nucleotide sequence encoding the AAV capsid polypeptide, e.g., the AAV capsid variant (e.g., an AAV capsid variant described herein), comprises a nucleotide sequence comprising at least one, two, three, four, five, six, or seven, but no more than ten different nucleotides relative to the nucleotide sequence of SEQ ID NO: 3663.

In some embodiments, the AAV capsid polypeptide, e.g., the AAV capsid variant, comprises an amino acid residue other than “A” at position 587 and/or an amino acid residue other than “Q” at position 588, numbered according to SEQ ID NO: 138. In some embodiments, the AAV capsid polypeptide, e.g., the AAV capsid variant, comprises one, two, three, or all of an amino acid other than A at position 589, an amino acid other than Q at position 590, an amino acid other than A at position 591, and/or an amino acid other than Q at position 592, relative to a reference sequence numbered according to the amino acid sequence of SEQ ID NO: 138. In some embodiments, the AAV capsid polypeptide, e.g., the AAV capsid variant, comprises one, two, or all of an amino acid other than T at position 593, an amino acid other than G at position 594, and/or an amino acid other than W at position 595, relative to a reference sequence numbered according to the amino acid sequence of SEQ ID NO: 138. In some embodiments, the AAV capsid polypeptide, e.g., the AAV capsid variant, comprises one, two, or all of an amino acid other than V at position 596, an amino acid other than Q at position 597, and/or an amino acid other than N at position 598, relative to a reference sequence numbered according to the amino acid sequence of SEQ ID NO: 138.

In some embodiments, the AAV capsid polypeptide, e.g., the AAV capsid variant, comprises the amino acid sequence of PLNGAVHLY (SEQ ID NO: 3648) wherein the amino acid sequence of PLNGAVHLY (SEQ ID NO: 3648) is present immediately subsequent to position 586, relative to a reference sequence numbered according to the amino acid sequence of SEQ ID NO: 138.

In some embodiments, the polypeptide, e.g., the AAV capsid variant, comprises the amino acid sequence of GGTLAVVSL (SEQ ID NO: 3654), wherein the amino acid sequence of GGTLAVVSL (SEQ ID NO: 3654) is present immediately subsequent to position 586, relative to a reference sequence numbered according to the amino acid sequence of SEQ ID NO: 138.

In some embodiments, the AAV capsid polypeptide, e.g., the AAV capsid variant, comprises the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138.

In some embodiments, the AAV capsid polypeptide, e.g., the AAV capsid variant, comprises the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.

In some embodiments, the AAV capsid polypeptide, e.g., the AAV capsid variant, further comprises a substitution at position K449, e.g., a K449R substitution, numbered according to SEQ ID NO: 138. In some embodiments, the AAV capsid variant further comprises a modification, e.g., an insertion, substitution, and/or deletion in loop I, II, IV, and/or VI.

In some embodiments, the AAV capsid polypeptide, e.g., the AAV capsid variant, further comprises an amino acid sequence comprising at least one, two or three modifications, e.g., substitutions (e.g., conservative substitutions), insertions or deletions, but not more than 30, 20 or 10 modifications relative to the amino acid sequence of SEQ ID NO: 138. In some embodiments, the AAV capsid polypeptide, e.g., the AAV capsid variant, further comprises an amino acid sequence having at least one, two or three but no more than 30, 20, or 10 different amino acids relative to the amino acid sequence of SEQ ID NO: 138. In some embodiments, the AAV capsid variant further comprises the amino acid sequence of SEQ ID NO: 138, or an amino acid sequence with at least 80% (e.g., at least about 85, 90, 95, 96, 97, 98, or 99%) sequence identity thereto. In some embodiments, the AAV capsid variant further comprises an amino acid sequence encoded by the nucleotide sequence of SEQ ID NO: 137, or a sequence with at least 80% (e.g., at least about 85, 90, 95, 96, 97, 98, or 99%) sequence identity thereto.

In some embodiments, an AAV capsid polypeptide, e.g., the AAV capsid variant, comprises immediately subsequent to position 586, 588, or 589, numbered relative to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety), at least 3, 4, 5, 6, 7, 8, 9, 10, 11, 12, 13, 14, 15, 16, 17, 18, or 19 consecutive amino acids of any of amino acid sequence provided in Tables 3, 20, 24 or 25. In some embodiments, the at least 3, 4, 5, 6, 7, 8, 9, 10, 11, 12, 13, 14, 15, 16, 17, 18, or 19 consecutive amino acids of any of amino acid sequence provided in Tables 3, 20, 24 or 25 replaces at least one, two, three, four, five, six, seven, eight, nine, ten, elven, or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the at least 3, 4, 5, 6, 7, 8, 9, 10, 11, 12, 13, 14, 15, 16, 17, 18, or 19 consecutive amino acids of any of amino acid sequence provided in Tables 3, 20, 24 or 25 replaces positions A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at one, two, three, four, five, six, seven, eight, nine, ten, eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises a modification, e.g., substitution, at one, two, three, four, five, six, seven, eight, nine, ten eleven or all of positions A587, Q588, A589, Q590, A591, Q592, T593, G594, W595, V596, Q597, and/or N598, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety). In some embodiments, the AAV capsid variant comprises a modification, e.g., substitution, at position A587, Q588, or both positions A587 and Q588, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 11, PHP.N, PHP.B, or an AAV serotype as provided in Table 6 of WO 2021/230987 (the contents of which are hereby incorporated by reference in their entirety).

In some embodiments, an AAV capsid polypeptide, e.g., an AAV capsid variant, of the present disclosure comprises an amino acid sequence as described herein, e.g. an amino acid sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 4, 5, and 23.

In some embodiments, an AAV capsid polypeptide, e.g. the AAV capsid variant, comprises a VP1, VP2, and/or VP3 protein comprising an amino acid sequence described herein, e.g. an amino acid sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 4, 5 and 23.

In some embodiments, an AAV capsid polypeptide, e.g., the AAV capsid variant, comprises an amino acid sequence encoded by a nucleotide sequence as described herein, e.g. a nucleotide sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 4 and 6.

In some embodiments, a polynucleotide encoding an AAV capsid polypeptide, e.g., an AAV capsid variant, of the present disclosure comprises a nucleotide sequence described herein, e.g. a nucleotide sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 4 and 6.

In some embodiments, insertion of a nucleic acid sequence, targeting nucleic acid sequence, or a peptide into a parent AAV sequence generates the non-limiting exemplary full length capsid sequences, e.g., an AAV capsid polypeptide, e.g., an AAV capsid variant, as described in Tables 4, 5, 6, and 23.

TABLE 4
Exemplary full length capsid sequences (VP1 with insert)

| | VP1 DNA | VP1 PRT | Peptide PRT | Peptide DNA |
|---|---|---|---|---|
| Serotype | SEQ ID NO: | SEQ ID NO: | SEQ ID NO: | SEQ ID NO: |
| TTD-001 | 3623 | 3636 | 3648 | 3660 |
| TTD-002 | 3624 or 3625 | 3637 | 3649 | 3661 |
| TTD-003 | 3626 | 3638 | 3650 | 3662 |
| TTD-004 | 3627 | 3639 | 3651 | 3663 |
| TTD-005 | 3628 | 3640 | 3652 | 3664 |
| TTD-006 | 3629 | 3641 | 3653 | 3665 |
| TTD-007 | 3630 | 3642 | 3654 | 3666 |
| TTD-008 | 3631 | 3643 | 3655 | 3667 |
| TTD-009 | 3632 | 3644 | 3656 | 3668 |
| TTD-010 | 3633 | 3645 | 3657 | 3669 |
| TTD-011 | 3634 | 3646 | 3658 | 3670 |
| TTD-012 | 3635 | 3647 | 3659 | 3671 |
| TTD-013 | 4 | 5 | 314 | 6 |
| TTD-014 | 7 | 8 | 566 | 9 |

TABLE 5
Exemplary full length capsid amino acid sequences

| | SEQ | |
|---|---|---|
| Name and | ID | |
| Annotation | NO: | Amino Acid Sequence |
| TTD-001 | 3636 | MAADGYLPDWLEDNLSEGIREWWALKPGAPQPKANQQHQDNARGLVLPG |
| 9mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 587 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 586); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSPL |
| | | NGAVHLYAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-002 | 3637 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ARDSPKGWQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-003 | 3638 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDVRMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-004 | 3639 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 589 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 588); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | IVMNSLKAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-005 | 3640 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ARESPRGLQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-006 | 3641 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ASFNDTRAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-007 | 3642 | MAADGYLPDWLEDNLSEGIREWWALKPGAPQPKANQQHQDNARGLVLPG |
| 9mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 587 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 586); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSGG |
| | | TLAVVSLAQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-008 | 3643 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYGLPKGPQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-009 | 3644 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | ASTGTLRLQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-010 | 3645 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDERMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-011 | 3646 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYSTDERKQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-012 | 3647 | MAADGYLPDWLEDNLSEGIREWWALKPGAPQPKANQQHQDNARGLVLPG |
| 7mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 590 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent | | PRDWQRLINNNWGFRPKRLNFKLFNIQVKEVTDNNGVKTIANNLTSTVQ |
| to 589); | | VFTDSDYQLPYVLGSAHEGCLPPFPADVFMIPQYGYLTLNDGSQAVGRS |
| 743 aa | | SFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVS |
| | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSAQ |
| | | AYVSSVKMQAQTGWVQNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-013 | 5 | MAADGYLPDWLEDNLSEGIREWWALKPGAPQPKANQQHQDNARGLVLPG |
| 9mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 587 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 586); | | VFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLID |
| position 604; | | QYLYYLSKTINGSGQNQQTLKFSVAGPSNMAVQGRNYIPGPSYRQQRVS |
| 743 aa | | TTVTQNNNSEFAWPGASSWALNGRNSLMNPGPAMASHKEGEDRFFPLSG |
| | | SLIFGKQGTGRDNVDADKVMITNEEEIKTTNPVATESYGQVATNHQSPL |
| | | NGAVHLYAQAQTGWVPNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |
| TTD-014 | 8 | MAADGYLPDWLEDNLSEGIREWWALKPGAPQPKANQQHQDNARGLVLPG |
| 9mer peptide | | YKYLGPGNGLDKGEPVNAADAAALEHDKAYDQQLKAGDNPYLKYNHADA |
| underlined, | | EFQERLKEDTSFGGNLGRAVFQAKKRLLEPLGLVEEAAKTAPGKKRPVE |
| starts at | | QSPQEPDSSAGIGKSGAQPAKKRLNFGQTGDTESVPDPQPIGEPPAAPS |
| position 587 | | GVGSLTMASGGGAPVADNNEGADGVGSSSGNWHCDSQWLGDRVITTSTR |
| (immediately | | TWALPTYNNHLYKQISNSTSGGSSNDNAYFGYSTPWGYFDFNRFHCHFS |
| subsequent to | | PRDWQRLINNNWGFRPKRLNFKLFNIQVKEVTDNNGVKTIANNLTSTVQ |
| position 586); | | VFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLID |
| positions 600, | | QYLYYLSKTINGSGQNQQTLKFSVAGPSNMAVQGRNYIPGPSYRQQRVS |
| 601, 602, and | | TTVTQNNNSEFAWPGASSWALNGRNSLMNPGPAMASHKEGEDRFFPLSG |
| 604 | | SLIFGKQGTGRDNVDADKVMITNEEEIKTTNPVATESYGQVATNHQSPL |
| 743 aa | | NGAVHLYAQAQLSPVKNQGILPGMVWQDRDVYLQGPIWAKIPHTDGNFH |
| | | PSPLMGGFGMKHPPPQILIKNTPVPADPPTAFNKDKLNSFITQYSTGQV |
| | | SVEIEWELQKENSKRWNPEIQYTSNYYKSNNVEFAVNTEGVYSEPRPIG |
| | | TRYLTRNL |

TABLE 6
Exemplary full length capsid nucleic acid sequences

| | SEQ | |
|---|---|---|
| Name and | ID | |
| Annotation | NO: | NT Sequence |
| TTD-001 | 3623 | atggctgccgatggttatcttccagattggctcgaggacaaccttagtgaaggaattcgc |
| 9mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtccgcttaatggtgccgtccatctttatgctcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-002 | 3624 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggctcgtgattctccgaagggttggcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| | 3625 | atggctgccgatggttatcttccagattggctcgaggacaaccttagtgaaggaattcgc |
| | | gagtggtgggctttgaaacctggagcccctcaacccaaggcaaatcaacaacatcaagac |
| | | aacgctcgaggtcttgtgcttccgggttacaaataccttggacccggcaacggactcgac |
| | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggctcgtgattctccgaagggttggcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcggatcctccaacggccttcaacaaggacaagctgaactctttcatcacccag |
| | | tattctactggccaagtcagcgtggagatcgagtgggagctgcagaaggaaaacagcaag |
| | | cgctggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-003 | 3626 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggcttattctacggatgtgaggatgcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-004 | 3627 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacagattgttatgaattcgttgaaggctcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-005 | 3628 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggctcgggagagtcctcgtgggctgcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-006 | 3629 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggctagttttaatgatactagggctcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-007 | 3630 | atggctgccgatggttatcttccagattggctcgaggacaaccttagtgaaggaattcgc |
| 9mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtggtggtacgttggccgtcgtgtcgcttgctcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-008 | 3631 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggcttatgggttgccgaagggtcctcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-009 | 3632 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggcttcgactgggacgcttcggcttcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-010 | 3633 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcgcaggcgtattcgacggatgagaggatgcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-011 | 3634 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcgcaggcgtattcgacggatgagaggaagcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-012 | 3635 | atggctgccgatggttatcttccagattggctcgaggacaaccttagtgaaggaattcgc |
| 7mer | | gagtggtgggctttgaaacctggagcccctcaacccaaggcaaatcaacaacatcaagac |
| peptide | | aacgctcgaggtcttgtgcttccgggttacaaataccttggacccggcaacggactcgac |
| underlined | | aagggggagccggtcaacgcagcagacgcggcggccctcgagcacgacaaggcctacgac |
| | | cagcagctcaaggccggagacaacccgtacctcaagtacaaccacgccgacgccgagttc |
| | | caggagcggctcaaagaagatacgtcttttgggggcaacctcgggcgagcagtcttccag |
| | | gccaaaaagaggcttcttgaacctcttggtctggttgaggaagcggctaagacggctcct |
| | | ggaaagaagaggcctgtagagcagtctcctcaggaaccggactcctccgcgggtattggc |
| | | aaatcgggtgcacagcccgctaaaaagagactcaatttcggtcagactggcgacacagag |
| | | tcagtcccagaccctcaaccaatcggagaacctcccgcagccccctcaggtgtgggatct |
| | | cttacaatggcttcaggtggtggcgcaccagtggcagacaataacgaaggtgccgatgga |
| | | gtgggtagttcctcgggaaattggcattgcgattcccaatggctgggggacagagtcatc |
| | | accaccagcacccgaacctgggccctgcccacctacaacaatcacctctacaagcaaatc |
| | | tccaacagcacatctggaggatcttcaaatgacaacgcctacttcggctacagcaccccc |
| | | tgggggtattttgacttcaacagattccactgccacttctcaccacgtgactggcagcga |
| | | ctcatcaacaacaactggggattccggcctaagcgactcaacttcaagctcttcaacatt |
| | | caggtcaaagaggttacggacaacaatggagtcaagaccatcgccaataaccttaccagc |
| | | acggtccaggtcttcacggactcagactatcagctcccgtacgtgctcgggtcggctcac |
| | | gagggctgcctcccgccgttcccagcggacgttttcatgattcctcagtacgggtatctg |
| | | acgcttaatgatggaagccaggccgtgggtcgttcgtccttttactgcctggaatatttc |
| | | ccgtcgcaaatgctaagaacgggtaacaacttccagttcagctacgagtttgagaacgta |
| | | cctttccatagcagctacgctcacagccaaagcctggaccgactaatgaatccactcatc |
| | | gaccaatacttgtactatctctcaaagactattaacggttctggacagaatcaacaaacg |
| | | ctaaaattcagtgtggccggacccagcaacatggctgtccagggaagaaactacatacct |
| | | ggacccagctaccgacaacaacgtgtctcaaccactgtgactcaaaacaacaacagcgaa |
| | | tttgcttggcctggagcttcttcttgggctctcaatggacgtaatagcttgatgaatcct |
| | | ggacctgctatggccagccacaaagaaggagaggaccgtttctttcctttgtctggatct |
| | | ttaatttttggcaaacaaggaactggaagagacaacgtggatgcggacaaagtcatgata |
| | | accaacgaagaagaaattaaaactactaacccggtagcaacggagtcctatggacaagtg |
| | | gccacaaaccaccagagtgcacaggcttatgtttcgtctgttaagatgcaggcgcagacc |
| | | ggctgggttcaaaaccaaggaatacttccgggtatggtttggcaggacagagatgtgtac |
| | | ctgcaaggacccatttgggccaaaattcctcacacggacggcaactttcacccttctccg |
| | | ctgatgggagggtttggaatgaagcacccgcctcctcagatcctcatcaaaaacacacct |
| | | gtacctgcCgatcctccaacggccttcaacaaggacaagctgaactctttcatcacccag |
| | | tattctactggccaagtcagcgtggagatcgagtgggagctgcagaaggaaaacagcaag |
| | | cgGtggaacccggagatccagtacacttccaactattacaagtctaataatgttgaattt |
| | | gctgttaatactgaaggtgtatatagtgaaccccgccccattggcaccagatacctgact |
| | | cgtaatctgtaa |
| TTD-013 | 5 | MAADGYLPDWLEDNLSEGIREWWALKPGAPQPKANQQHQDNARGLVLPGYKYLG |
| 9mer | | PGNGLDKGEPVNAADAAALEHDKAYDQQLKAGDNPYLKYNHADAEFQERLKEDT |
| peptide | | SFGGNLGRAVFQAKKRLLEPLGLVEEAAKTAPGKKRPVEQSPQEPDSSAGIGKS |
| underlined, | | GAQPAKKRLNFGQTGDTESVPDPQPIGEPPAAPSGVGSLTMASGGGAPVADNNE |
| starts at | | GADGVGSSSGNWHCDSQWLGDRVITTSTRTWALPTYNNHLYKQISNSTSGGSSN |
| position 587 | | DNAYFGYSTPWGYFDFNRFHCHFSPRDWQRLINNNWGFRPKRLNFKLFNIQVKE |
| (immediately | | VTDNNGVKTIANNLTSTVQVFTDSDYQLPYVLGSAHEGCLPPFPADVFMIPQYG |
| subsequent | | YLTLNDGSQAVGRSSFYCLEYFPSQMLRTGNNFQFSYEFENVPFHSSYAHSQSL |
| to position | | DRLMNPLIDQYLYYLSKTINGSGQNQQTLKFSVAGPSNMAVQGRNYIPGPSYRQ |
| 586); | | QRVSTTVTQNNNSEFAWPGASSWALNGRNSLMNPGPAMASHKEGEDRFFPLSGS |
| modification | | LIFGKQGTGRDNVDADKVMITNEEEIKTTNPVATESYGQVATNHQSPLNGAVHL |
| at position | | YAQAQTGWVPNQGILPGMVWQDRDVYLQGPIWAKIPHTDGNFHPSPLMGGFGMK |
| 604; | | HPPPQILIKNTPVPADPPTAFNKDKLNSFITQYSTGQVSVEIEWELQKENSKRW |
| 743 aa | | NPEIQYTSNYYKSNNVEFAVNTEGVYSEPRPIGTRYLTRNL |
| TTD-014 | 8 | MAADGYLPDWLEDNLSEGIREWWALKPGAPQPKANQQHQDNARGLVLPGYKYLG |
| 9mer | | PGNGLDKGEPVNAADAAALEHDKAYDQQLKAGDNPYLKYNHADAEFQERLKEDT |
| peptide | | SFGGNLGRAVFQAKKRLLEPLGLVEEAAKTAPGKKRPVEQSPQEPDSSAGIGKS |
| underlined, | | GAQPAKKRLNFGQTGDTESVPDPQPIGEPPAAPSGVGSLTMASGGGAPVADNNE |
| starts at | | GADGVGSSSGNWHCDSQWLGDRVITTSTRTWALPTYNNHLYKQISNSTSGGSSN |
| position 587 | | DNAYFGYSTPWGYFDFNRFHCHFSPRDWQRLINNNWGFRPKRLNFKLFNIQVKE |
| (immediately | | VTDNNGVKTIANNLTSTVQVFTDSDYQLPYVLGSAHEGCLPPFPADVFMIPQYG |
| subsequent | | YLTLNDGSQAVGRSSFYCLEYFPSQMLRTGNNFQFSYEFENVPFHSSYAHSQSL |
| to position | | DRLMNPLIDQYLYYLSKTINGSGQNQQTLKFSVAGPSNMAVQGRNYIPGPSYRQ |
| 586); | | QRVSTTVTQNNNSEFAWPGASSWALNGRNSLMNPGPAMASHKEGEDRFFPLSGS |
| modifica- | | LIFGKQGTGRDNVDADKVMITNEEEIKTTNPVATESYGQVATNHQSPLNGAVHL |
| tions at | | YAQAQLSPVKNQGILPGMVWQDRDVYLQGPIWAKIPHTDGNFHPSPLMGGFGMK |
| positions | | HPPPQILIKNTPVPADPPTAFNKDKLNSFITQYSTGQVSVEIEWELQKENSKRW |
| 600, 601, | | NPEIQYTSNYYKSNNVEFAVNTEGVYSEPRPIGTRYLTRNL |
| 602, and 604 | | |
| 743 aa | | |

In some embodiments, an AAV capsid polypeptide, e.g., an AAV capsid variant, described herein comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3636, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3639, or an amino acid sequence with at least 80% (e.g., at least about 85, 90, 95, 96, 97, 98, or 99%) sequence identity thereto.

In some embodiments, the polynucleotide encoding an AAV capsid polypeptide, e.g., AAV capsid variant, described herein comprises the nucleotide sequence of any one of SEQ ID NOs: 3623-3635, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3623, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3627, or a nucleotide sequence with at least 80% (e.g., at least about 85, 90, 95, 96, 97, 98, or 99%) sequence identity thereto. In some embodiments, the nucleic acid sequence encoding an AAV capsid polypeptide, e.g., an AAV capsid variant, described herein is codon optimized.

In some embodiments, the AAV capsid polypeptide, e.g., the AAV capsid variant, comprises a VP2 protein comprising the amino acid sequence corresponding to positions 138-743, of any one of SEQ ID NOs: 3636-3647, or a sequence with at least 80% (e.g., at least about 85, 90, 95, 96, 97, 98, or 99%) sequence identity thereto. In some embodiments, the AAV capsid comprises a VP3 protein comprising the amino acid sequence corresponding to positions 203-743, of any one of SEQ ID NOs: 3636-3647, or a sequence with at least 80% (e.g., at least about 85, 90, 95, 96, 97, 98, or 99%) sequence identity thereto.

In some embodiments, the AAV capsid polypeptide, e.g., the AAV capsid variant, e.g., an AAV capsid variant described herein, comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of any one of SEQ ID NOs: 3636-3647. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of any one of SEQ ID NOs: 3636-3647.

In some embodiments, the AAV capsid polypeptide, e.g., the AAV capsid variant, e.g., an AAV capsid variant described herein, comprises the amino acid sequence of SEQ ID NO: 3636, or an amino acid sequence with at least 80% (e.g., at least about 85, 90, 95, 96, 97, 98, or 99%) sequence identity thereto. In some embodiments, the AAV capsid polypeptide, e.g., the AAV capsid variant, e.g., an AAV capsid variant described herein, comprises an amino acid sequence comprising at least one, two, or three modifications, substitutions (e.g., conservative substitutions), insertions, or deletions, but not more than 30, 20 or 10 modifications, substitutions (e.g., conservative substitutions), insertions, or deletions relative to the amino acid sequence of SEQ ID NO: 3636. In some embodiments, the AAV capsid polypeptide, e.g., the AAV capsid variant, e.g., an AAV capsid variant described herein, comprises an amino acid sequence having at least one, two, or three but no more than 30, 20, or 10 different amino acids relative to the amino acid sequence of SEQ ID NO: 3636.

In some embodiments, an AAV capsid polypeptide, e.g., an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid polypeptide, e.g., an AAV capsid variant, described herein transduces a brain region, e.g., selected from dentate nucleus, cerebellar cortex, cerebral cortex, brain stem, hippocampus, thalamus and putamen. In some embodiments, the level of transduction of said brain region is at least 5, 10, 50, 100, 200, 500, 1,000, 2,000, 5,000, or 10,000-fold greater as compared to a reference sequence of SEQ ID NO: 138.

In some embodiments, an AAV capsid polypeptide, e.g., an AAV capsid variant, described herein is enriched at least about 5, 6, 7, 8, 9, or 10-fold, in the brain compared to a reference sequence of SEQ ID NO: 138. In some embodiments, an AAV capsid variant described herein is enriched at least about 20, 30, 40, or 50-fold in the brain compared to a reference sequence of SEQ ID NO: 138. In some embodiments, an AAV capsid variant described herein is enriched at least about 100, 200, 300, or 400-fold in the brain compared to a reference sequence of SEQ ID NO: 138.

In some embodiments, an AAV capsid polypeptide, e.g., an AAV capsid variant, described herein delivers an increased level of viral genomes to a brain region. In some embodiments, the level of viral genomes is increased by at least 5, 10, 20, 30, 40 or 50-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a brain region. In some embodiments, the level of the payload is increased by at least 5, 10, 50, 100, 200, 500, 1,000, 2,000, 5,000, or 10,000-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid polypeptide, e.g., an AAV capsid variant, described herein delivers an increased level of a payload to a spinal cord region. In some embodiments, the level of the payload is increased by at least 10, 20, 50, 100, 200, 300, 400, 500, 600, 700, 800 or 900-fold, as compared to a reference sequence of SEQ ID NO: 138. In some embodiments, the spinal cord region comprises a cervical, thoracic, and/or lumbar region.

In some embodiments, an AAV capsid polypeptide, e.g., an AAV capsid variant, described herein shows preferential transduction in a brain region relative to the transduction in the dorsal root ganglia (DRG).

In some embodiments, an AAV capsid polypeptide, e.g., an AAV capsid variant, described herein has an increased tropism for a muscle cell or tissue, e.g., a heart cell or tissue, relative to the tropism of a reference sequence comprising the amino acid sequence of SEQ ID NO: 138. In some embodiments, the AAV capsid variant delivers an increased level of a payload to a muscle region. In some embodiments, the payload is increased by at least 10, 15, 20, 30, or 40-fold, as compared to a reference sequence of SEQ ID NO: 138. In some embodiments, the muscle region comprises a heart muscle, quadriceps muscle, and/or a diaphragm muscle region. In some embodiments, the muscle region comprises a heart muscle region, e.g., a heart atrium muscle region or a heart ventricle muscle region.

In some embodiments, an AAV capsid polypeptide, e.g., an AAV capsid variant described herein results in greater than 1, 2, 5, 10, 20, 30, 40, 50, or 100 reads per sample, e.g., when analyzed by an NGS sequencing assay.

In some embodiments, an AAV capsid polypeptide, e.g., an AAV capsid variant, of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant described comprises a modification, e.g., substitution (e.g., conservative substitution), insertion, or deletion, that results in one or more of the following properties: (1) reduced tropism in the liver; (2) de-targeted expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), N498 (e.g., N498Y or N4981), W503 (e.g., W530R or W530A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498/(e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N297A), Y446 (e.g., Y446A), and W503 (e.g., W530R or W530A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5(3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in its entirety.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific “SEQ ID NO:” (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

Also provided herein are polynucleotide sequences encoding any of the AAV capsid variants described above and AAV particles, vectors, and cells comprising the same.

### Viral Genome

In some aspects, the AAV particle of the present disclosure serves as an expression vector comprising a viral genome which encodes a SOD1 targeting siRNA. In some embodiments, expression vectors are not limited to AAV and may be adenovirus, retrovirus, lentivirus, plasmid, vector, or any variant thereof.

In some embodiments, an AAV particle, e.g., an AAV particle for the vectorized delivery of a siRNA described herein (e.g., a SOD1 targeting siRNA), comprises a viral genome, e.g., an AAV viral genome (e.g., a vector genome or AAV vector genome). In some embodiments, the viral genome, e.g., the AAV viral genome, further comprises an inverted terminal repeat (ITR) region, an enhancer, a promoter, an intron region, a Kozak sequence, an exon region, a nucleic acid encoding a transgene encoding a payload (e.g., a SOD1 targeting siRNA described herein), a nucleotide sequence encoding a miR binding site, a poly A signal region, or a combination thereof.

In some embodiments, an AAV particle as described herein comprising an AAV capsid polypeptide, e.g., AAV capsid variant, described herein, may be used for the delivery of a viral genome to a tissue (e.g., CNS, DRG, and/or muscle). In some embodiments, an AAV particle comprising an AAV capsid polypeptide, e.g., an AAV capsid variant, described herein can be used for delivery of a viral genome to a tissue or cell, e.g., CNS, DRG, or muscle cell or tissue. In some embodiments, an AAV particle of the present disclosure is a recombinant AAV particle. In some embodiments, an AAV particle of the present disclosure is an isolated AAV particle.

The viral genome may encode any payload, such as but not limited to a polypeptide (e.g., a therapeutic polypeptide), an antibody, an enzyme, an RNAi agent and/or components of a gene editing system (e.g., a polynucleotide, e.g., a SOD1 targeting siRNA duplex). In one embodiment, the AAV particles described herein are used to deliver a payload to cells of the CNS, after intravenous delivery. In another embodiment, the AAV particles described herein are used to deliver a payload to cells of the DRG, after intravenous delivery. In some embodiments, the AAV particles described herein are used to deliver a payload to cells of a muscle, e.g., a heart muscle, after intravenous delivery.

In some embodiments, a viral genome of an AAV particle comprising an AAV capsid polypeptide, e.g., an AAV capsid variant, as described herein, comprises a nucleic acid comprising a transgene encoding a payload (e.g., a SOD1 targeting siRNA duplex). In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein (e.g., comprising an AAV capsid variant described herein) may comprise a regulatory element (e.g., promoter), untranslated regions (UTR), a miR binding site a polyadenylation sequence (polyA), a filler or stuffer sequence, an intron, and/or a linker sequence, e.g., for enhancing transgene expression.

In some embodiments, the viral genome components are selected and/or engineered for expression of a payload in a target tissue (e.g., a CNS tissue, a muscle tissue (e.g., heart), or DRG).

### Viral Genome Component: Inverted Terminal Repeats (ITRs)

In some embodiments, the AAV particle described herein (e.g., an AAV particle comprising an AAV capsid polypeptide, e.g., an AAV capsid variant described herein), comprises a viral genome (e.g., a viral genome encoding a SOD1 siRNA described herein), with at least one ITR and a payload region. In one embodiment, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes as described herein may be comprised of naturally occurring polynucleotide sequences or recombinantly derived polynucleotide sequences.

The ITRs may be derived from the same serotype as the capsid polypeptide, e.g., capsid variant, selected from any of the known serotypes, or a derivative thereof. The ITR may be of a different serotype than the capsid. In one embodiment, the AAV particle has more than one ITR. In a non-limiting example, the AAV particle has a viral genome comprising two ITRs. In one embodiment, the ITRs are of the same serotype as one another. In another embodiment, the ITRs are of different serotypes. Non-limiting examples include zero, one or both of the ITRs having the same serotype as the capsid. In one embodiment both ITRs of the viral genome of the AAV particle are AAV2 ITRs.

In some embodiments, the ITR sequence (e.g., 5′ ITR sequence) comprises the nucleotide sequence of SEQ ID NO: 126. In some embodiments, the ITR sequence (e.g., 5′ ITR sequence) comprises a nucleotide sequence having at least 90% identity, at least 95% identity, at least 98% identity, or at least 99% identity to SEQ ID NO: 126. In some embodiments, the ITR sequence (e.g., 5′ ITR sequence) has a nucleotide sequence with at 1, 2, 3, 4, 5, 6, 7, 8, 9, 10, 1-5, 1-10, 1-20, 10-20, 1-30, 10-30, or 20-30 nucleotide modifications (e.g., substitutions, insertions, and/or deletions) relative to SEQ ID NO: 126.

In some embodiments, the ITR sequence (e.g., 3′ ITR sequence) comprises the nucleotide sequence of SEQ ID NO: 130. In some embodiments, the ITR sequence (e.g., 3′ ITR sequence) comprises a nucleotide sequence having at least 90% identity, at least 95% identity, at least 98% identity, or at least 99% identity to SEQ ID NO: 130. In some embodiments, the ITR sequence (e.g., 3′ ITR sequence) has a nucleotide sequence with at 1, 2, 3, 4, 5, 6, 7, 8, 9, 10, 1-5, 1-10, 1-20, 10-20, 1-30, 10-30, or 20-30 nucleotide modifications (e.g., substitutions, insertions, and/or deletions) relative to SEQ ID NO: 130.

### Viral Genome Component: Promoters

In one embodiments, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid comprising a transgene encoding a payload, wherein the transgene is operably linked to a promoter. In some embodiments, the promoter is a species' specific promoter, an inducible promoter, tissue-specific, or cell cycle-specific (Parr et al., Nat. Med. 3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is deemed to be efficient when it drives expression of the payload encoded by the viral genome of the AAV particle.

In some embodiments, the promoter is a promoter deemed to be efficient when it drives expression in a cell being targeted.

In some embodiments, the promoter is a promoter having a tropism for a cell being targeted.

In some embodiments, the promoter drives expression of the payload (e.g., the SOD1 targeting siRNA described herein) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a selected for sustained expression of a payload in tissues and/or cells of the central or peripheral nervous system.

In some embodiments, the promoter is a ubiquitous promoter, e.g., capable of expression in multiple tissues. In some embodiments the promoter is a human elongation factor 1α-subunit (EF1α) promoter or a variant or fragment thereof, the cytomegalovirus (CMV) immediate-early enhancer and/or promoter, the chicken β-actin (CBA) promoter or a variant or fragment thereof and its derivative CAG, β glucuronidase (GUSB) promoter, phosphoglycerate kinase (PGK) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a cell or tissue of the central nervous system. In some embodiments, the promoter is synapsin (Syn) promoter or a variant or fragment thereof, glutamate vesicular transporter (VGLUT) promoter, vesicular GABA transporter (VGAT) promoter, parvalbumin (PV) promoter, sodium channel Naᵥ 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In certain embodiments, the promoter is a variant or fragment of any of the promoters described herein.

In some embodiments, the promoter of the viral genome described herein (e.g., comprised within an AAV particle comprising an AAV capsid variant described herein) comprises an EF-1α promoter or a fragment or variant thereof, a chicken beta-actin (CBA) promoter or a fragment or variant thereof, a PGK promoter or a fragment or variant thereof, an insulin promoter or a fragment or variant thereof, a synapsin (SYN) promoter or a fragment or variant thereof, e.g., as provided in Table 7. In some embodiments, the promoter comprises the nucleotide sequence of any one of SEQ ID NOs: 4000-4026, a nucleotide sequence comprising at least one, two, or three but no more than four modifications (e.g., substitutions, insertions, and/or deletions) relative to the nucleotide sequence of SEQ ID NOs: 4000-4026, or a nucleotide sequence with at least 70% (e.g., 80, 85%, 90%, 95%, 96%, 97%, 98%, or 99%) sequence identity to any one of SEQ ID NOs: 4000-4026.

TABLE 7
Exemplary Promoter Sequences and Variants

| | | SEQ |
|---|---|---|
| | | ID |
| Description | Sequence | NO: |
| EF1a | CGTGAGGCTCCGGTGCCCGTCAGTGGGCAGAGCGCACATCGCCCACAGTCCCCGAGA | 4000 |
| Promoter | AGTTGGGGGGAGGGGTCGGCAATTGAACCGGTGCCTAGAGAAGGTGGCGCGGGGTAA | |
| (intron | ACTGGGAAAGTGATGTCGTGTACTGGCTCCGCCTTTTTCCCGAGGGTGGGGGAGAAC | |
| underlined) | CGTATATAAGTGCAGTAGTCGCCGTGAACGTTCTTTTTCGCAACGGGTTTGCCGCCA | |
| | GAACACAGGTAAGTGCCGTGTGTGGTTCCCGCGGGCCTGGCCTCTTTACGGGTTATG | |
| | GCCCTTGCGTGCCTTGAATTACTTCCACCTGGCTGCAGTACGTGATTCTTGATCCCG | |
| | AGCTTCGGGTTGGAAGTGGGTGGGAGAGTTCGAGGCCTTGCGCTTAAGGAGCCCCTT | |
| | CGCCTCGTGCTTGAGTTGAGGCCTGGCCTGGGCGCTGGGGCCGCCGCGTGCGAATCT | |
| | GGTGGCACCTTCGCGCCTGTCTCGCTGCTTTCGATAAGTCTCTAGCCATTTAAAATT | |
| | TTTGATGACCTGCTGCGACGCTTTTTTTCTGGCAAGATAGTCTTGTAAATGCGGGCC | |
| | AAGATCTGCACACTGGTATTTCGGTTTTTGGGGCCGCGGGCGGCGACGGGGCCCGTG | |
| | CGTCCCAGCGCACATGTTCGGCGAGGCGGGGCCTGCGAGCGCGGCCACCGAGAATCG | |
| | GACGGGGGTAGTCTCAAGCTGGCCGGCCTGCTCTGGTGCCTGGCCTCGCGCCGCCGT | |
| | GTATCGCCCCGCCCTGGGCGGCAAGGCTGGCCCGGTCGGCACCAGTTGCGTGAGCGG | |
| | AAAGATGGCCGCTTCCCGGCCCTGCTGCAGGGAGCTCAAAATGGAGGACGCGGCGCT | |
| | CGGGAGAGCGGGCGGGTGAGTCACCCACACAAAGGAAAAGGGCCTTTCCGTCCTCAG | |
| | CCGTCGCTTCATGTGACTCCACGGAGTACCGGGCGCCGTCCAGGCACCTCGATTAGT | |
| | TCTCGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGATGT | |
| | AATTCTCCTTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGA | |
| miniEF1a | GCCCGTCAGTGGGCAGAGCGCACATCGCCCACAGTCCCCGAGAAGTTGGGGGGAGGG | 4001 |
| | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGCGTAAG | |
| Promoter | GCATG | |
| Variant 1 | | |
| Promoter | GGTGGAGAAGAGCATG | 4003 |
| Variant 2 | | |
| Promoter | GTCATCACTGAGGTGGAGAAGAGCATG | 4004 |
| Variant 3 | | |
| Promoter | CGTGAG | |
| Variant 4 | | |
| Promoter | GT | |
| Variant 5 | | |
| Promoter | GCTCCGGT | |
| Variant 6 | | |
| Promoter | GCCCGTCAGTGGGCAGAGCGCACATCGCCCACAGTCCCCGAGAAGTTGGGGGGAGGG | 4008 |
| Variant 19 | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACAG | |
| Promoter | GCCCGTCAGTGGGCAGAGCGCACATCGCCCACAGTCCCCGAGAAGTTGGGGGGAGGG | 4009 |
| Variant 20 | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGC | |
| Promoter | GTAAG | |
| Variant 7 | | |
| Promoter | GTGCCCGTCAGTGGGCAGAGCGCACATCGCCCACAGTCCCCGAGAAGTTGGGGGGAG | 4011 |
| Variant 8 | GGGTCGGCAATTGAACCGGTGCCTAGAGAAGGTGGCGCGGGGTAAACTGGGAAAGTG | |
| | ATGTCGTGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACACGCGTAA | |
| | G | |
| Promoter | GCTCCGGTGCCCGTCAGTGGGCAGAGCGCACATCGCCCACAGTCCCCGAGAAGTTGG | 4012 |
| Variant 9 | GGGGAGGGGTCGGCAATTGAACCGGTGCCTAGAGAAGGTGGCGCGGGGTAAACTGGG | |
| | AAAGTGATGTCGTGTACTGGCTCCGCCTTTTTCCCGAGGGTGGGGGAGAACCGTATA | |
| | TAAGTGCAGTAGTCGCCGTGAACGTTCTTTTTCGCAACGGGTTTGCCGCCAGAACAC | |
| | GCGTAAG | |
| Promoter | CGTGAGGCTCCGGTGCCCGTCAGTGGGCAGAGCGCACATCGCCCACAGTCCCCGAGA | 4013 |
| Variant 10 | AGTTGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACGCGTAAG | |
| Promoter | CGTGAGGCTCCGGTGCCCGTCAGTGGGCAGAGCGCACATCGCCCACAGTCCCCGAGA | 4014 |
| Variant 11 | AGTTGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACAG | |
| Promoter | GCATGCGTGAGGCTCCGGTGCCCGTCAGTGGGCAGAGCGCACATCGCCCACAGTCCC | 4015 |
| Variant 12 | CGAGAAGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACGCGTAAG | |
| Promoter | GCATGCGTGAGGCTCCGGTGCCCGTCAGTGGGCAGAGCGCACATCGCCCACAGTCCC | 4016 |
| Variant 13 | CGAGAAGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACAG | |
| Promoter | GGTGGAGAAGAGCATGCGTGAGGCTCCGGTGCCCGTCAGTGGGCAGAGCGCACATCG | 4017 |
| Variant 14 | CCCACAGTCCCCGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACGCGTAAG | |
| Promoter | GGTGGAGAAGAGCATGCGTGAGGCTCCGGTGCCCGTCAGTGGGCAGAGCGCACATCG | 4018 |
| Variant 15 | CCCACAGTCCCCGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACAG | |
| Promoter | GTCATCACTGAGGTGGAGAAGAGCATGCGTGAGGCTCCGGTGCCCGTCAGTGGGCAG | 4019 |
| Variant 16 | AGCGCACATCGCCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGGCAG | 4020 |
| Variant 18 | AGCGCACATCGCCCACAGTCCCCGAGAAGITGGGGGGAGGGGTCGGCAATTGAACCG | |
| | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACAG | |
| Chicken | CGACATTGATTATTGACTAGTTATTAATAGTAATCAATTACGGGGTCATTAGTTCAT | 4021 |
| beta- | AGCCCATATATGGAGTTCCGCGTTACATAACTTACGGTAAATGGCCCGCCTGGCTGA | |
| actin (CBA) | CCGCCCAACGACCCCCGCCCATTGACGTCAATAATGACGTATGTTCCCATAGTAACG | |
| promoter | CCAATAGGGACTTTCCATTGACGTCAATGGGTGGAGTATTTACGGTAAACTGCCCAC | |
| | TTGGCAGTACATCAAGTGTATCATATGCCAAGTACGCCCCCTATTGACGTCAATGAC | |
| | GGTAAATGGCCCGCCTGGCATTATGCCCAGTACATGACCTTATGGGACTTTCCTACT | |
| | TGGCAGTACATCTACGTATTAGTCATCGCTATTACCATGTCGAGGCCACGTTCTGCT | |
| | TCACTCTCCCCATCTCCCCCCCCTCCCCACCCCCAATTTTGTATTTATTTATTTTTT | |
| | AATTATTTTGTGCAGCGATGGGGGCGGGGGGGGGGGGCGCGCGCCAGGCGGGGGGGG | |
| | GCGGGGCGAGGGGCGGGGCGGGGCGAGGCGGAGAGGTGCGGCGGCAGCCAATCAGAG | |
| | CGGCGCGCTCCGAAAGTTTCCTTTTATGGCGAGGCGGCGGCGGCGGCGGCCCTATAA | |
| | AAAGCGAAGCGCGCGGCGGGCGGGAGC | |
| Mini-CBA | CCACGTTCTGCTTCACTCTCCCCATCTCCCCCCCCTCCCCACCCCCAATTTTGTATT | 4022 |
| promoter | TATTTATTTTTTAATTATTTTGTGCAGCGATGGGGGCGGGGGGGGGGGGCGCGCGCC | |
| | AGGCGGGGCGGGGCGGGGCGAGGGGCGGGGCGGGGCGAGGCGGAGAGGTGCGGCGGC | |
| | AGCCAATCAGAGCGGCGCGCTCCGAAAGTTTCCTTTTATGGCGAGGCGGCGGCGGCG | |
| | GCGGCCCTATAAAAAGCGAAGCGCGCGGCGGG | |
| PGK promoter | GGGTAGGGGAGGCGCTTTTCCCAAGGCAGTCTGGAGCATGCGCTTTAGCAGCCCCGC | 4023 |
| | TGGGCACTTGGCGCTACACAAGTGGCCTCTGGCCTCGCACACATTCCACATCCACCG | |
| | GTAGGCGCCAACCGGCTCCGTTCTTTGGTGGCCCCTTCGCGCCACCTTCTACTCCTC | |
| | CCCTAGTCAGGAAGTTCCCCCCCGCCCCGCAGCTCGCGTCGTGCAGGACGTGACAAA | |
| | TGGAAGTAGCACGTCTCACTAGTCTCGTGCAGATGGACAGCACCGCTGAGCAATGGA | |
| | AGCGGGTAGGCCTTTGGGGCAGCGGCCAATAGCAGCTTTGCTCCTTCGCTTTCTGGG | |
| | CTCAGAGGCTGGGAAGGGGTGGGTCCGGGGGCGGGCTCAGGGGCGGGCTCAGGGGCG | |
| | GGGCGGGCGCCCGAAGGTCCTCCGGAGGCCCGGCATTCTGCACGCTTCAAAAGCGCA | |
| | CGTCTGCCGCGCTGTTCTCCTCTTCCTCATCTCCGGGCCTTTCG | |
| Insulin | CCCTAATGGGCCAGGCGGCAGGGGTTGAGAGGTAGGGGAGATGGGCTCTGAGACTAT | 4024 |
| promoter | AAAGCCAGCGGGGGCCCAGCAGCCCTC | |
| fragment | | |
| Human | CGCGTGGGGTTATTTCTCTACTTTCGTGTCTCTGAGTGTGCTTCCAGTGCCCCCCTC | 4025 |
| synapsin | CCCCCAAAAAATGCCTTCTGAGTTGAATATCAACACTACAAACCTAGTATCTGCAGA | |
| (SYN) | GGGCCCTGCGTATGAGTGCAAGTGGGTTTTAGGACCAGGATGAGGCGGGGTGGGGGT | |
| promoter | GCCTACCTGACGACCGACCCCGACCCACTGGACAAGCACCCAACCCCCATTCCCCAA | |
| (long) | ATTGCGCATCCCCTATCAGAGAGGGGGAGGGGAAACAGGATGCGGCGAGGCGCGTGC | |
| | GCACTGCCAGCTTCAGCACCGCGGACAGTGCCTTCGCCCCCGCCTGGCGGCGCGCGC | |
| | CACCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGCAC | |
| | CACGCGAGGCGCGAGATAGGGGGGCACGGGCGCGACCATCTGCGCTGCGGCGCCGGC | |
| | GACTCAGCGCTGCCTCAGTCTGCGGTGGGCAGCGGAGGAGTCGTGTCGTGCCTGAGA | |
| | GCGCAGCTGTGCTCCTGGGCACCGCGCAGTCCGCCCCCGCGGCTCCTGGCCAGACCA | |
| | CCCCTAGGACCCCCTGCCCCAAGTCGCAGCC | |
| Human SYN | TAGTATCTGCAGAGGGCCCTGCGTATGAGTGCAAGTGGGTTTTAGGACCAGGATGAG | 4026 |
| promoter | GCGGGGTGGGGGTGCCTACCTGACGACCGACCCCGACCCACTGGACAAGCACCCAAC | |
| (short) | CCCCATTCCCCAAATTGCGCATCCCCTATCAGAGAGGGGGAGGGGAAACAGGATGCG | |
| | GCGAGGCGCGTGCGCACTGCCAGCTTCAGCACCGCGGACAGTGCCTTCGCCCCCGCC | |
| | TGGCGGCGCGCGCCACCGCCGCCTCAGCACTGAAGGCGCGCTGACGTCACTCGCCGG | |
| | TCCCCCGCAAACTCCCCTTCCCGGCCACCTTGGTCGCGTCCGCGCCGCCGCCGGCCC | |
| | AGCCGGACCGCACCACGCGAGGCGCGAGATAGGGGGGCACGGGCGCGACCATCTGCG | |
| | CTGCGGCGCCGGCGACTCAGCGCTGCCTCAGTCTGCGGTGGGCAGCGGAGGAGTCGT | |
| | GTCGTGCCTGAGAGCGCAGCTGTGCTCCTGGGCACCGCGCAGTCCGCCCCCGCGGCT | |
| | CCTGGCCAGACCACCCCTAGGACCCCCTGCCCCAAGTCGCAGCC | |

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG (SEQ ID NO: 2622), where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) (SEQ ID NO: 2623) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

In some embodiments, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In some embodiments, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

The viral genome encoding a SOD1 siRNA described herein may comprise at least one polyadenylation sequence. In some embodiments, the viral genome of the AAV particle (e.g., an AAV particle comprising an AAV capsid polypeptide, e.g., an AAV capsid variant, described herein) comprises a polyadenylation sequence between the 3′ end of the payload encoding region and the 5′ end of the 3′ITR.

In some embodiments, the AAV particle comprises a nucleic acid sequence encoding an siRNA molecule may be located upstream of the polyadenylation sequence in an expression vector. Further, the AAV particle comprises a nucleic acid sequence encoding an siRNA molecule may be located downstream of a promoter such as, but not limited to, CMV, U6, CAG, CBA or a CBA promoter with a SV40 intron or a human beta globin intron in an expression vector.

In some embodiments, the AAV particle comprises a rabbit globin polyadenylation (polyA) signal sequence (rBGpA).

In some embodiments, the AAV particle comprises a human growth hormone (hGH) polyadenylation (polyA) signal sequence.

In some embodiments, the polyadenylation signal sequence comprises the nucleotide sequence of SEQ ID NO: 129 or 4027, a nucleotide sequence comprising at least one, two, or three but no more than four modifications (e.g., substitutions, insertions, and/or deletions) relative to the nucleotide sequence of SEQ ID NO: 129 or 4027, or a nucleotide sequence with at least 70% (e.g., 80, 85%, 90%, 95%, 96%, 97%, 98%, or 99%) sequence identity to SEQ ID NO: 129 or 4027.

TABLE 8
Exemplary Poly A sequences

| | | SEQ |
|---|---|---|
| | | ID |
| Description | Sequence | NO: |
| rBGpA polyA | GATCTTTTTCCCTCTGCCAAAAATTATGGGGACATCATGAAGCCCCTTGAGCAT | 129 |
| signal sequence | CTGACTTCTGGCTAATAAAGGAAATTTATTTTCATTGCAATAGTGTGTTGGAAT | |
| | TTTTTGTGTCTCTCACTCG | |
| Human growth | GGGTGGCATCCCTGTGACCCCTCCCCAGTGCCTCTCCTGGCCCTGGAAGTTGCC | 4027 |
| hormone | ACTCCAGTGCCCACCAGCCTTGTCCTAATAAAATTAAGTTGCATCATTTTGTCT | |
| (hGH) polyA | GACTAGGTGTCCTTCTATAATATTATGGGGTGGAGGGGGGTGGTATGGAGCAAG | |
| signal sequence | GGGCAAGTTGGGAAGACAACCTGTAGGGCCTGCGGGGTCTATTGGGAACCAAGC | |
| | TGGAGTGCAGTGGCACAATCTTGGCTCACTGCAATCTCCGCCTCCTGGGTTCAA | |
| | GCGATTCTCCTGCCTCAGCCTCCCGAGTTGTTGGGATTCCAGGCATGCATGACC | |
| | AGGCTCAGCTAATTTTTGTTTTTTTGGTAGAGACGGGGTTTCACCATATTGGCC | |
| | AGGCTGGTCTCCAACTCCTAATCTCAGGTGATCTACCCACCTTGGCCTCCCAAA | |
| | TTGCTGGGATTACAGGCGTGAACCACTGCTCCCTTCCCTGTCCTT | |

### Viral Genome Component: Exons and Introns

In some embodiments, the viral genome encoding a SOD1 targeting siRNA described herein, comprises at least one element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an exon and/or intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps). In one embodiment, the intron is a beta globulin intron.

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

In some embodiments, the viral genome encoding a SOD1 targeting siRNA described herein comprises an intron comprising the nucleotide sequence of SEQ ID NO: 4044, a nucleotide sequence comprising at least one, two, or three but no more than four modifications (e.g., substitutions, insertions, and/or deletions), relative to the nucleotide sequence of SEQ ID NO: 4044, or a nucleotide sequence with at least 70% (e.g., 80, 85%, 90%, 95%, 96%, 97%, 98%, or 99%) sequence identity to SEQ ID NO: 4044.

In some embodiments, the intron is positioned 3′ relative to a 5′ITR and/or promoter, and 5′ relative to a nucleotide sequence encoding a modulatory polynucleotide (e.g., a SOD1 targeting RNA agent, e.g., a siRNA duplex), a poly A signal sequence, and/or a 3′ITR.

In some embodiments, the viral genome encoding a SOD1 targeting siRNA described herein comprises one or more exons (e.g., 1, 2, 3, 4, 5, 6, 7, or 8 or more exons). In some embodiments, the viral genome comprises a first exon, a second exon, or both a first and a second exon. In some embodiments, the exon has a length of about 40, 50, 51, 52, 53, 54, 55, 56, 57, 58, 59, 60, 70, 80, 90, 100, 110, 120, 130, 131, 132, 133, 134, 135, 136, 137, 138, 139, 140, 150, 160, 170, 180, 190, 200, 210, 220, 230, 240, 250, 260, 270, 280, 290, 300, 310, 320, 330, 340, 350, 360, 370, 380, 390, or 400 nucleotides. In some embodiments, the exon has a length of about 40-200, 40-190, 40-180, 40-170, 40-160, 40-150, 40-140, 40-130, 40-120, 40-110, 40-100, 40-90, 40-80, 40-70, 40-60, 80-200, 80-190, 80-180, 80-170, 80-160, 80-150, 80-140, 80-130, 80-120, 80-110, 80-100, 80-90, 120-200, 120-190, 120-180, 120-170, 120-160, 120-150, 120-140, 120-130, 150-200, 150-190, 150-180, 150-170, 150-160, 170-200, 170-190, 170-180, 180-200, 180-190, 190-200, 185-190, 130-140, 130-135, 50-60, or 50-55 nucleotides.

In some embodiments, the first or second exon comprises the nucleotide sequence of any one of SEQ ID NO: 4045-4048, a nucleotide sequence comprising at least one, two, or three but no more than four modifications, e.g., substitutions, insertions, or deletions, relative to the nucleotide sequence of any one of SEQ ID NO: 4045-4048, or a nucleotide sequence with at least 80% (e.g., 85%, 90%, 95%, 96%, 97%, 98%, or 99%) sequence identity to any one of SEQ ID NO: 4045-4048.

In some embodiments, the viral genome comprises both a first exon and a second exon, wherein:
(a) the first exon comprises the nucleotide sequence of SEQ ID NO: 4045 or 4046, a nucleotide sequence comprising at least one, two, or three but no more than four modifications, e.g., substitutions, insertions, or deletions, relative to the nucleotide sequence of SEQ ID NO: 4045 or 4046, or a nucleotide sequence with at least 80% (e.g., 85%, 90%, 95%, 96%, 97%, 98%, or 99%) sequence identity to SEQ ID NO: 4045 or 4046; and
(b) the second exon comprises the nucleotide sequence of SEQ ID NO: 4047 or 4048, a nucleotide sequence comprising at least one, two, or three but no more than four modifications, e.g., substitutions, insertions, or deletions, relative to the nucleotide sequence of SEQ ID NO: 4047 or 4048, or a nucleotide sequence with at least 80% (e.g., 85%, 90%, 95%, 96%, 97%, 98%, or 99%) sequence identity to SEQ ID NO: 4047 or 4048.

In some embodiments, one or more or all exons are codon optimized. In some embodiments, one or more or all of the CpG sequences in the one or more or all exons are depleted (e.g., altered to remove CpG sequences, such as by substitution, insertion, or deletion).

In some embodiments, the viral genome comprises a first exon, an intron (e.g., an intron comprising the sequence of SEQ ID NO: 4044 or a variant thereof), and a second exon (referred to herein as an “exon-intron-exon cassette”).

In some embodiments, the viral genome comprises a first exon, an intron (e.g., an intron comprising the sequence of SEQ ID NO: 4044 or a variant thereof), and a second exon, wherein the intron is located 3′ relative to the first exon and 5′ relative to the second exon.

In some embodiments, the exon-intron-exon cassette comprises the nucleotide sequence of SEQ ID NO: 4042 or 4043, a nucleotide sequence comprising at least one, two, or three but no more than four modifications, e.g., substitutions, insertions, or deletions, relative to the nucleotide sequence of SEQ ID NO: 4042 or 4043, or a nucleotide sequence with at least 80% (e.g., 85%, 90%, 95%, 96%, 97%, 98%, or 99%) sequence identity to SEQ ID NO: 4042 or 4043.

In some embodiments, the exon-intron-exon cassette is positioned 3′ relative to the 5′ITR and/or promoter, and 5′ relative to the nucleotide sequence encoding the modulatory polynucleotide (e.g., a SOD1 targeting RNA agent, e.g., a siRNA duplex), poly A signal sequence, and/or 3′ITR.

In some embodiments, the nucleotide sequence encoding the modulatory polynucleotide (e.g., a SOD1 targeting RNA agent, e.g., a siRNA duplex) is located within (e.g., inserted into) the intron of the exon-intron-exon cassette.

In some embodiments, the nucleotide sequence encoding the modulatory polynucleotide (e.g., a SOD1 targeting RNA agent, e.g., a siRNA duplex) replaces all or a portion of the intron in the exon-intron-exon cassette.

TABLE 9
Exemplary exon, intron, and exon-intron-exon cassette sequences

| | | SEQ |
|---|---|---|
| | | ID |
| Description | Sequence | NO: |
| Human beta | TCAGATCGCCTGGAGACGCCATCCACGCTGTTTTGACCTCCATAGAAGACACCG | 4042 |
| globulin (hBG) | GGACCGATCCAGCCTCCGCGGATTCGAATCCCGGCCGGGAACGGTGCATTGGAA | |
| exon-intron- | CGCGGATTCCCCGTGCCAAGAGTGACGTAAGTACCGCCTATAGAGTCTATAGGC | |
| exon cassette | CCACAAAAAATGCTTTCTTCTTTTAATATACTTTTTTGTTTATCTTATTTCTAA | |
| [CpG | TACTTTCCCTAATCTCTTTCTTTCAGGGCAATAATGATACAATGTATCATGCCT | |
| underlined] | CTTTGCACCATTCTAAAGAATAACAGTGATAATTTCTGGGTTAAGGCAATAGCA | |
| | ATATTTCTGCATATAAATATTTCTGCATATAAATTGTAACTGATGTAAGAGGTT | |
| | TCATATTGCTAATAGCAGCTACAATCCAGCTACCATTCTGCTTTTATTTTATGG | |
| | TTGGGATAAGGCTGGATTATTCTGAGTCCAAGCTAGGCCCTTTTGCTAATCATG | |
| | TTCATACCTCTTATCTTCCTCCCACAGCTCCTGGGCAACGTGCTGGTCTGTGTG | |
| | CTGGCCCATCACTTTGGCAAAGAATT | |
| hβG exon- | TCAGATCACCTGGAGACACCATCCACACTGTTTTGACCTCCATAGAAGACACCA | 4043 |
| intron-exon | GGACCAATCCAGCCTCCACAGATTCCAATCCCAGCCAGGAACAGTGCATTGGAA | |
| cassette [CpG | CACAGATTCCCCTTGCCAAGAGTGACGTAAGTACCGCCTATAGAGTCTATAGGC | |
| depleted] | CCACAAAAAATGCTTTCTTCTTTTAATATACTTTTTTGTTTATCTTATTTCTAA | |
| | TACTTTCCCTAATCTCTTTCTTTCAGGGCAATAATGATACAATGTATCATGCCT | |
| | CTTTGCACCATTCTAAAGAATAACAGTGATAATTTCTGGGTTAAGGCAATAGCA | |
| | ATATTTCTGCATATAAATATTTCTGCATATAAATTGTAACTGATGTAAGAGGTT | |
| | TCATATTGCTAATAGCAGCTACAATCCAGCTACCATTCTGCTTTTATTTTATGG | |
| | TTGGGATAAGGCTGGATTATTCTGAGTCCAAGCTAGGCCCTTTTGCTAATCATG | |
| | TTCATACCTCTTATCTTCCTCCCACAGCTCCTGGGCAACCTGCTGGTCTGTGTG | |
| | CTGGCCCATCACTTTGGCAAAGAATT | |
| hβG intron | GTAAGTACCGCCTATAGAGTCTATAGGCCCACAAAAAATGCTTTCTTCTTTTAA | 4044 |
| | TATACTTTTTTGTTTATCTTATTTCTAATACTTTCCCTAATCTCTTTCTTTCAG | |
| | GGCAATAATGATACAATGTATCATGCCTCTTTGCACCATTCTAAAGAATAACAG | |
| | TGATAATTTCTGGGTTAAGGCAATAGCAATATTTCTGCATATAAATATTTCTGC | |
| | ATATAAATTGTAACTGATGTAAGAGGTTTCATATTGCTAATAGCAGCTACAATC | |
| | CAGCTACCATTCTGCTTTTATTTTATGGTTGGGATAAGGCTGGATTATTCTGAG | |
| | TCCAAGCTAGGCCCTTTTGCTAATCATGTTCATACCTCTTATCTTCCTCCCACA | |
| | G | |
| hβG exon (first | TCAGATCGCCTGGAGACGCCATCCACGCTGTTTTGACCTCCATAGAAGACACCG | 4045 |
| exon) [CpG | GGACCGATCCAGCCTCCGCGGATTCGAATCCCGGCCGGGAACGGTGCATTGGAA | |
| underlined] | CGCGGATTCCCCGTGCCAAGAGTGAC | |
| hBG exon (first | TCAGATCACCTGGAGACACCATCCACACTGTTTTGACCTCCATAGAAGACACCA | 4046 |
| exon) [CpG | GGACCAATCCAGCCTCCACAGATTCCAATCCCAGCCAGGAACAGTGCATTGGAA | |
| depleted] | CACAGATTCCCCTTGCCAAGAGTGAC | |
| hβG exon | CTCCTGGGCAACGTGCTGGTCTGTGTGCTGGCCCATCACTTTGGCAAAGAATT | 4047 |
| (second exon) | | |
| [CpG | | |
| underlined] | | |
| hβG exon | CTCCTGGGCAACCTGCTGGTCTGTGTGCTGGCCCATCACTTTGGCAAAGAATT | 4048 |
| (second exon) | | |
| [CpG depleted] | | |

In some embodiments, the encoded siRNA molecule may be located downstream of a promoter in an expression vector such as, but not limited to, CMV, U6, H1, CBA, CAG, or a CBA promoter with an intron such as SV40 or others known in the art. Further, the encoded siRNA molecule may also be located upstream of the polyadenylation sequence in an expression vector.

In some embodiments, the nucleotide sequence encoding the siRNA may be located within an intron (e.g., inserted into an intron), or replace all or a portion of an intron sequence.

In some embodiments, the nucleotide sequence encoding the siRNA may be located within an intron (e.g., inserted into an intron), or replace all or a portion of an intron sequence, of an exon-intron-exon cassette (e.g., the exon-intron-exon cassette of SEQ ID NO: 4042 or 4043).

### Viral Genome Component: Stuffer Sequence

In some embodiments, the viral genome encoding a SOD1 targeting siRNA described herein, comprises at least one element to improve packaging efficiency and expression, such as a stuffer or filler sequence. Non-limiting examples of a stuffer sequence includes albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

In certain embodiments, the filler sequence may comprise a region or a portion of a lentivirus.

In some embodiments, the filler sequence may comprise a region or a portion of the albumin gene. In certain embodiments, the filler sequence may comprise a region or a portion of the human albumin gene (NCBI Reference Sequence: NG_009291.1).

Viral Genome Component: miRNA

In some embodiments, the viral genome comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed. In some embodiments, the miR binding site modulates, e.g., reduces, expression of the encoded siRNA for SOD1 in a cell or tissue of the DRG, liver, hematopoietic lineage, or a combination thereof.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, the encoded miR binding site series comprises 4 copies of a miR binding site. In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer is about 8 nucleotides in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complementary), to the miR in the host cell. In some embodiments, the sequence complementary to the nucleotide sequence encoding the miR binding site comprises at least 1, 2, 3, 4, or 5 mismatches or no more than 6, 7, 8, 9, or 10 mismatches relative to the corresponding miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complementary to the miR in the host cell.

In some embodiments, the encoded miR binding site or the encoded miR binding site series is about 10 to about 125 nucleotides in length, e.g., about 10 to 50 nucleotides, 10 to 100 nucleotides, 50 to 100 nucleotides, 50 to 125 nucleotides, or 100 to 125 nucleotides in length. In some embodiments, an encoded miR binding site or the encoded miR binding site series is about 7 to about 28 nucleotides in length, e.g., about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., full complementary or partially complementary) to the seed sequence of a miRNA (e.g., a miR122, a miR142, a miR-1, a miR183).

In some embodiments, the encoded miR binding site is complementary (e.g., fully complementary or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 1865), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 1865, e.g., wherein the modification can result in a mismatch between the encoded miR binding site and the corresponding miRNA. In some embodiments, the viral genome comprises at least 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATT GTCACACTCCA (SEQ ID NO: 1866), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 1866, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully complementary or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site is complementary (e.g., fully complementary or partially complementary) to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 1869), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 1869, e.g., wherein the modification can result in a mismatch between the encoded miR binding site and the corresponding miRNA. In some embodiments, the viral genome comprises at least 3, 4, or 5 copies of an encoded miR-142-3p binding site, e.g., an encoded miR-142-3p binding site series. In some embodiments, the at least 3, 4, or 5 copies (e.g., 4 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary (e.g., fully complementary or partially complementary) to a miR expressed in expressed in a DRG neuron. In some embodiments, the encoded miR binding site comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 1847), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 1847, e.g., wherein the modification can result in a mismatch between the encoded miR binding site and the corresponding miRNA. In some embodiments, the sequence complementary (e.g., fully complementary or partially complementary) to the seed sequence corresponds to the double underlined of the encoded miR-183 binding site sequence. In some embodiments, the viral genome comprises at least comprises at least 3, 4, or 5 copies (e.g., 4 copies) of the encoded miR183 binding site, e.g. an encoded miR183 binding site. In some embodiments, the viral genome comprises at least comprises 4 copies of the encoded miR183 binding site, e.g. an encoded miR183 binding site comprising 4 copies of a miR183 binding site. In some embodiments, the at least 3, 4, or 5 copies (e.g., 4 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or encoded miR binding site series comprises a miR182 binding site sequence. In some embodiments, the encoded miR182 binding site comprises, the nucleotide sequence of AGTGTGAGTTCTACCATTGCCAAA (SEQ ID NO: 1867), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 1867, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 1868), a sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 1868, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

### Viral Genome Size

In one embodiment, the AAV particle described herein (e.g., an AAV particle comprising an AAV capsid polypeptide, e.g., an AAV capsid variant), may comprise a single-stranded or double-stranded viral genome. The size of the viral genome may be small, medium, large or the maximum size. As described above, the viral genome may comprise a promoter and a polyA tail.

In one embodiment, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In one embodiment, the viral genome may be a small double stranded viral genome. A small double stranded viral genome may be 1.3 to 1.7 kb in size such as, but not limited to, about 1.3, 1.4, 1.5, 1.6, and 1.7 kb in size.

In one embodiment, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In one embodiment, the viral genome may be a medium double stranded viral genome. A medium double stranded viral genome may be 1.8 to 2.1 kb in size such as, but not limited to, about 1.8, 1.9, 2.0, and 2.1 kb in size.

In one embodiment, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In one embodiment, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

In some embodiments, an AAV particle, e.g., an AAV particle for the vectorized delivery of an RNA agent, e.g., a siRNA, e.g., a SOD1 targeting siRNA described herein, comprises a payload. In some embodiments, an AAV particle, e.g., an AAV particle for the vectorized delivery of a siRNA described herein (e.g., a SOD1 targeting siRNA), comprises a viral genome encoding a payload. In some embodiments, the viral genome comprises a promoter operably linked to a nucleic acid comprising a transgene encoding a payload. In some embodiments, the payload comprises a siRNA.

In some embodiments, the disclosure herein provides constructs that allow for improved expression of a SOD1 targeting siRNA delivered by gene therapy vectors.

In some embodiments, the disclosure provides constructs that allow for improved biodistribution of a SOD1 targeting siRNA delivered by gene therapy vectors.

In some embodiments, the disclosure provides constructs that allow for improved sub-cellular distribution or trafficking of a SOD1 targeting siRNA delivered by gene therapy vectors.

In some embodiments, the disclosure provides constructs that allow for improved trafficking of a SOD1 targeting siRNA to cells in the central nervous system, e.g., the brain and the spinal cord, delivered by gene therapy vectors.

In some aspects, the present disclosure relates to a composition containing or comprising a nucleic acid sequence encoding a SOD1 targeting siRNA or functional fragment or variants thereof and methods of administering the composition in vitro or in vivo in a subject, e.g., a humans and/or an animal model of disease, e.g., a disease related to expression of SOD1.

AAV particles of the present disclosure may comprise a nucleic acid sequence encoding at least one “payload.” As used herein, “payload” or “payload region” refers to one or more polynucleotides or polynucleotide regions encoded by or within a viral genome or an expression product of such polynucleotide or polynucleotide region, e.g., a transgene, a polynucleotide encoding a SOD1 targeting polynucleotide (e.g., a SOD1 targeting RNA agent, e.g., a siRNA, e.g., a siRNA duplex for inhibiting expression of SOD1) or fragment or variant thereof. The payload may comprise any nucleic acid known in the art that is useful for the expression (by supplementation of the SOD1 siRNA or gene replacement using a modulatory nucleic acid) of the SOD1 targeting siRNA in a target cell transduced or contacted with the AAV particle carrying the payload.

The payload construct may comprise a combination of coding and non-coding nucleic acid sequences.

Any segment, fragment, or the entirety of the viral genome and therein, the payload region, may be codon optimized.

In some embodiments, the viral genome encodes more than one payload. As a non-limiting example, a viral genome encoding more than one payload may be replicated and packaged into a viral particle. A target cell transduced with a viral particle comprising more than one payload may express each of the payloads in a single cell.

In some embodiments, the viral genome may encode a coding or non-coding RNA. In certain embodiments, the adeno-associated viral vector particle further comprises at least one cis-element selected from the group consisting of a Kozak sequence, a backbone sequence, and an intron sequence.

In some embodiments, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the viral genome include, but are not limited to, mammalian proteins. In certain embodiments, the AAV particle contains a viral genome that encodes a SOD1 targeting polynucleotide (e.g., a SOD1 targeting RNAi, e.g., a siRNA duplex for inhibiting expression of SOD1), or a fragment or variant thereof. The AAV particles described herein may be useful in the fields of human disease, veterinary applications, and a variety of in vivo and in vitro settings.

In some embodiments, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In some embodiments, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise a SOD1 siRNA or a polynucleotide encoding a SOD1 siRNA to treat SOD1-related disorders.

In some embodiments, the payload encodes a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ, or ex vivo.

The components of an mRNA include, but are not limited to, a coding region, a 5′-UTR (untranslated region), a 3′-UTR, a 5′-cap and a poly-A tail. In some embodiments, the encoded mRNA or any portion of the AAV genome may be codon optimized.

A payload construct encoding a payload may comprise or encode a selectable marker. A selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP). In some embodiments, a payload construct encoding a selectable marker may comprise a human influenza hemagglutinin (HA) tag.

In certain embodiments, a nucleic acid for expression of a payload in a target cell will be incorporated into the viral genome and located between two ITR sequences.

In some embodiments, a payload construct further comprises a nucleic acid sequence encoding a peptide that binds to the cation-independent mannose 6-phosphate (M6P) receptor (CI-MPR) with high affinity, as described in Int'l Pat. App. Pub. No. WO2019213180A1, the disclosure of which is incorporated herein by reference in its entirety. The peptide that binds CI-MPR can be, e.g., an IGF2 peptide or variant thereof. Binding of CI-MPR can facilitate cellular uptake or delivery and intracellular or sub-cellular targeting of therapeutic proteins provided by gene therapy vectors.

The payload region may be constructed in such a way as to reflect a region similar to or mirroring the natural organization of an mRNA, e.g., the mRNA encoded by SOD1.

### Modulatory Polynucleotides

In some embodiments, modulatory polynucleotides, e.g., RNA or DNA molecules, may be used to treat neurodegenerative disease, e.g., amyotrophic lateral sclerosis (ALS). As used herein, a “modulatory polynucleotide” is any nucleic acid sequence(s) which functions to modulate (either increase or decrease) the level or amount of a target gene, e.g., mRNA or protein levels.

In some embodiments, the modulatory polynucleotide comprises a nucleotide sequence encoding a pri-miRNA, a pre-miRNA, miRNA, or a siRNA. In some embodiments, the pri-miRNA or pre-miRNA is cleaved by cellular enzymes (e.g., Dicer) to produce a miRNA or siRNA. In some embodiments, the modulatory polynucleotide comprises a nucleotide sequence encoding a siRNA within a pri-miRNA or pre-miRNA scaffold sequence. In such cases, cleavage of the pre-miRNA or pri-miRNA by cellular enzymes (e.g., Dicer) produces a siRNA duplex.

In some embodiments, the modulatory polynucleotides may comprise at least one nucleic acid sequence encoding at least one siRNA molecule. The nucleic acids may, independently if there is more than one, encode 1, 2, 3, 4, 5, 6, 7, 8, 9, or more than 9 siRNA molecules.

In some embodiments, the molecular scaffold may be a natural pri-miRNA scaffold located downstream of a CMV promoter. As a non-limiting example, the natural pri-miRNA scaffold comprises all or a portion of the human miR155 scaffold. Methods for selection of a molecular scaffold and modulatory polynucleotide are known in the art, including, for example, U.S. Patent Application Publication US20210254103, which is hereby incorporated by reference in its entirety.

In certain embodiments, the modulatory polynucleotide is designed using at least one of the following properties: loop variant, seed mismatch/bulge/wobble variant, stem mismatch, loop variant and vassal stem mismatch variant, seed mismatch and basal stem mismatch variant, stem mismatch and basal stem mismatch variant, seed wobble and basal stem wobble variant, or a stem sequence variant.

The present disclosure relates, in part, to RNA interfering (RNAi) induced inhibition of gene expression for treating neurodegenerative disorders. Provided are siRNA duplexes or dsRNA that target SOD1 gene. Such siRNA duplexes or dsRNA can silence SOD1 gene expression in cells, for example, motor neurons, therefore, ameliorating symptoms of ALS such as motor death and muscle atrophy. The SOD1 siRNA may be encoded in polynucleotides of a recombinant AAV genome.

siRNA duplexes or dsRNA targeting a specific mRNA may be designed and synthesized as part of a target SOD1 targeting polynucleotide in vitro and introduced into cells for activating RNAi process.

siRNA Molecules

The present disclosure relates to RNA interference (RNAi) induced inhibition of gene expression for treating neurodegenerative disorders. Provided herein are RNA agents, e.g., siRNA duplexes or encoded dsRNA that target the gene of interest (referred to herein collectively as “siRNA molecules”). Such siRNA duplexes or encoded dsRNA can reduce or silence gene expression in cells, such as, but not limited to, medium spiny neurons, cortical neurons and/or astrocytes.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′region of the miRNA which is called the seed region, and other base pairs with its 3′region. miRNA mediated down regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The term “double-stranded RNA” or “dsRNA,” as used herein, refers to an RNAi that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA, e.g., through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-25 base pairs, 15-26 base pairs, 15-23 base pairs, 15-22 base pairs, 15-21 base pairs, 15-20 base pairs, 15-19 base pairs, 15-18 base pairs, 15-17 base pairs, 17-22 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”, or “loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

In another embodiment, the RNA agent may be a “single-stranded siRNA” that is introduced into a cell or organism to inhibit a target mRNA. Single-stranded RNAi agents bind to the RISC endonuclease Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein (e.g., sequences provided in Table 10) may be used as a single-stranded siRNA as described herein.

In another aspect, the RNA agent is a “single-stranded antisense RNA molecule”. A single-stranded antisense RNA molecule is complementary to a sequence within the target mRNA. Single-stranded antisense RNA molecules can inhibit translation in a stoichiometric manner by base pairing to the mRNA and physically obstructing the translation machinery, see Dias, N. et al, (2002) Mol Cancer Ther 1:347-355. Alternatively, the single-stranded antisense molecules inhibit a target mRNA by hydridizing to the target and cleaving the target through an RNaseH cleavage event. The single-stranded antisense RNA molecule may be about 10 to about 30 nucleotides in length and have a sequence that is complementary to a target sequence. For example, the single-stranded antisense RNA molecule may comprise a sequence that is at least about 10, 11, 12, 13, 14, 15, 16, 17, 18, 19, 20, or more contiguous nucleotides from any one of the antisense nucleotide sequences described herein, e.g., sequences provided in Table 10.

RNAi molecules which were designed to target against a nucleic acid sequence that encodes poly-glutamine repeat proteins which cause poly-glutamine expansion diseases such as Huntington's Disease, are described in U.S. Pat. Nos. 9,169,483 and 9,181,544 and International Patent Publication No. WO2015179525, the content of each of which is herein incorporated by reference in their entirety. U.S. Pat. Nos. 9,169,483 and 9,181,544 and International Patent Publication No. WO2015179525 each provide isolated RNA duplexes comprising a first strand of RNA (e.g., 15 contiguous nucleotides) and second strand of RNA (e.g., complementary to at least 12 contiguous nucleotides of the first strand) where the RNA duplex is about 15 to 30 base pairs in length. The first strand of RNA and second strand of RNA may be operably linked by an RNA loop (˜4 to 50 nucleotides) to form a hairpin structure which may be inserted into an expression cassette. Non-limiting examples of loop portions include SEQ ID NO: 9-14 of U.S. Pat. No. 9,169,483, the content of which is herein incorporated by reference in its entirety. Non-limiting examples of strands of RNA which may be used, either full sequence or part of the sequence, to form RNA duplexes include SEQ ID NO: 1-8 of U.S. Pat. No. 9,169,483 and SEQ ID NO: 1-11, 33-59, 208-210, 213-215 and 218-221 of U.S. Pat. No. 9,181,544, the contents of each of which is herein incorporated by reference in its entirety. Non-limiting examples of RNAi molecules include SEQ ID NOs: 1-8 of U.S. Pat. No. 9,169,483, SEQ ID NOs: 1-11, 33-59, 208-210, 213-215 and 218-221 of U.S. Pat. No. 9,181,544 and SEQ ID NOs: 1, 6, 7, and 35-38 of International Patent Publication No. WO2015/179525, the contents of each of which is herein incorporated by reference in their entirety.

In vitro synthetized siRNA molecules may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

Any of the foregoing molecules may be encoded by a viral genome.

Design and Sequences of siRNA Duplexes Targeting SOD1 Gene

In some embodiments, the SOD1 targeting polynucleotide is a single-stranded antisense RNAi molecule, a single-stranded siRNA, or a double-stranded RNAi (e.g., a siRNA duplex) that inhibits expression of SOD1. In some embodiments, the SOD1 targeting polynucleotide is a siRNA duplex comprising a sense strand and an antisense strand.

The present disclosure provides small interfering RNA (siRNA) duplexes (and modulatory polynucleotides encoding them) that target mRNA to interfere with SOD1 gene expression and/or SOD1 protein production.

As used herein, the term “nucleotide overhang” refers to at least one unpaired nucleotide that protrudes from the duplex structure of an RNAi, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA.

The encoded siRNA duplex of the present disclosure contains an antisense strand and a sense strand hybridized together forming a duplex structure, wherein the antisense strand is complementary to the nucleic acid sequence of the targeted SOD1 gene, and wherein the sense strand is homologous to the nucleic acid sequence of the targeted SOD1 gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

In some embodiments, the SOD1 targeting polynucleotide is a siRNA duplex comprising a sense strand and an antisense strand.

The siRNAs included herein encompass a dsRNA having an RNA strand (the antisense strand) having a region, e.g., a region that is 30 nucleotides or less, generally 19-24, nucleotides in length, that is substantially complementary (e.g., at least 70%, 75%, 80%, 85%, 90%, 95%, 99% or 100% complementary) to at least part of an mRNA transcript of the SOD1 gene (e.g., a human variant of the SOD1 gene).

In some embodiments, the antisense sequence of the siRNA (e.g., dsRNA) targets the nucleotides on the SOD1 transcript (e.g., mRNA). In some embodiments, the antisense sequence comprises or consists of a sequence that is fully complementary or substantially complementary (e.g., at least 70%, 75%, 80%, 85%, 90%, 95%, 99% or 100% complementary) to the nucleotides of the SOD1 transcript.

In some embodiments, the siRNA (e.g., dsRNA) described herein comprises an antisense strand having a region that is substantially complementary (e.g., at least 70%, 75%, 80%, 85%, 90%, 95%, 99% or 100% complementary) to a region of a human SOD1 gene. The siRNA may target any location of the SOD1 mRNA coding sequence, as well as the non-coding regions.

In embodiments, SOD1 comprises a wild type SOD1 gene, mRNA, and/or protein; a mutated SOD1 gene, mRNA, and/or protein comprising at least one mutation; or a combination thereof.

In some embodiments, the siRNA (e.g., dsRNA) described herein comprises an antisense strand having a region that is substantially complementary (e.g., at least 70%, 75%, 80%, 85%, 90%, 95%, 99% or 100% complementary) to a region of a mutant SOD1 gene. More than 100 SOD1 mutations have been identified. Most of these mutations produce a single amino acid replacement in the superoxide dismutase enzyme's chain of amino acids. A common mutation, which occurs in 50 percent of American patients with type 1 amyotrophic lateral sclerosis, is the replacement of alanine with valine at position 4 in the amino acid chain (Ala4Val).

In some embodiments, the region that is substantially complementary may be 30 nucleotides or less (e.g., 29, 28, 27, 26, 25, 24, 23, 22, 21, 20, 19, 18, 17, 16, or 15 nucleotides in length). In some embodiments, the region of complementarity may range from 15, 16, 17, 18, 19, 20, 21, 22, 23, 24, 25, 26, 27, 28, or 29 nucleotides up to 16, 17, 18, 19, 20, 21, 22, 23, 24, 25, 26, 27, 28, 29, or 30 nucleotides in length.

Some guidelines for designing siRNAs have been proposed in the art. These guidelines generally recommend generating a 19-nucleotide duplexed region, symmetric 2-3 nucleotide 3′overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing the SOD1 gene expression may be readily designed.

According to the present disclosure, siRNA molecules (e.g., siRNA duplexes or encoded dsRNA) that target the SOD1 gene are designed. Such siRNA molecules can specifically, suppress SOD1 gene expression and protein production. In some aspects, the siRNA molecules are designed and used to selectively “knock out” SOD1 gene variants in cells, i.e., mutated SOD1 transcripts that are identified in patients with ALS disease. In some aspects, the siRNA molecules are designed and used to selectively “knock down” SOD1 gene variants in cells. In other aspects, the siRNA molecules are able to inhibit or suppress both the wild type and mutated SOD1 gene.

In some embodiments, an siRNA molecule of the present disclosure comprises a sense strand and a complementary antisense strand in which both strands are hybridized together to form a duplex structure. The antisense strand has sufficient complementarity to the SOD1 mRNA sequence to direct target-specific RNAi, i.e., the siRNA molecule has a sequence sufficient to trigger the destruction of the target mRNA by the RNAi machinery or process.

In certain embodiments, an siRNA molecule of the present disclosure comprises a sense strand and a complementary antisense strand in which both strands are hybridized together to form a duplex structure and where the start site of the hybridization to the SOD1 mRNA is between nucleotide 15 and 1000 on the SOD1 mRNA sequence. As a non-limiting example, the start site may be between nucleotide 15-25, 15-50, 15-75, 15-100, 100-150, 150-200, 200-250, 250-300, 300-350, 350-400, 400-450, 450-500, 500-550, 550-600, 600-650, 650-700, 700-70, 750-800, 800-850, 850-900, 900-950, and 950-1000 on the SOD1 mRNA sequence.

In some embodiments, the antisense strand and target SOD1 mRNA sequences have 100% complementarity. The antisense strand may be complementary to any part of the target SOD1 mRNA sequence. Alternatively, the antisense strand may have 0, 1, 2, or 3 mismatches or no more than 4, 5, or 6 mismatches to the target SOD1 mRNA sequences. In some embodiments, the mismatched nucleotides are contiguous. In some embodiments, the mismatched nucleotides are non-contiguous. In some embodiments, the mismatched nucleotides occur outside the SOD1 target mRNA binding site, such as at one or both ends of the antisense strand. As a non-limiting example, the antisense strand and the SOD1 mRNA sequence have at least 80%, 81%, 82%, 83%, 84%, 85%, 86%, 87%, 88%, 89%, 90%, 91%, 92%, 93%, 94%, 95%, 96%, 97%, 98% or 99% or at least 80-90%, 80-95%, 80-99%, 90-95%, 90-99% or 95-99% complementarity.

In certain embodiments, an siRNA or dsRNA includes at least two sequences that are complementary to each other. The siRNA or dsRNA may have a sense strand sequence and an antisense strand sequence that are 100% complementary to each other, or alternatively, they may have 1, 2, 3, 4, or more mismatches. These mismatches may be contiguous, or alternatively, they may be non-contiguous throughout the sequence. In some embodiments, the mismatched nucleotides are located at the ends of the sense and antisense sequences. In some embodiments, the mismatched nucleotides of the sense and antisense sequence are on the 5′ end of the sequences, and/or on the 3′ end of the sense and antisense sequences, and/or on both the 5′ and 3′ ends of the sense and antisense sequences.

According to the present disclosure, the siRNA molecule targeting SOD1 has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In certain embodiments, each strand of the siRNA molecule has a length from about 19 to 25, 19 to 24 or 19 to 21 nucleotides. In some embodiments, each strand of the siRNA molecule has a length ranging from 15, 16, 17, 18, 19, 20, 21, 22, 23, 24, 25, 26, 27, 28, 29, or 30 nucleotides up to 16, 17, 18, 19, 20, 21, 22, 23, 24, 25, 26, 27, 28, 29, 30 or 31 nucleotides. In some embodiments, at least one strand of the siRNA molecule (e.g., the sense strand and/or the antisense strand) is 19, 20, 21, 22, 23, 24, or 25 nucleotides in length.

In some embodiments, the siRNA molecules of the present disclosure targeting SOD1 can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In certain embodiments, the siRNA molecules of the present disclosure targeting SOD1 may comprise a nucleotide sequence such as, but not limited to, the antisense (guide) sequences in Table 10 or a fragment or variant thereof. As a non-limiting example, the antisense sequence used in the siRNA molecule of the present disclosure may comprises at least 15 (e.g., at least 16, 17, 18, 19, 20, 21, 22, or 23) contiguous nucleotides differing by no more than 3 (e.g., by no more than 0, 1 or 2) nucleotides from an antisense sequence listed in any one of the duplexes in Table 10. As a non-limiting example, the antisense sequence used in the siRNA molecule of the present disclosure is at least 80%, 81%, 82%, 83%, 84%, 85%, 86%, 87%, 88%, 89%, 90%, 91%, 92%, 93%, 94%, 95%, 96%, 97%, 98% or 99% or at least 70-80%, 70-90%, 70-95%, 70-99%, 80-90%, 80-95%, 80-99%, 90-95%, 90-99% or 95-99% identical to an antisense nucleotide sequence in Table 10. As another non-limiting example, the antisense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 10. As yet another non-limiting example, the antisense sequence used in the siRNA molecule of the present disclosure comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the antisense strand sequences in Table 10.

In certain embodiments, the siRNA molecules of the present disclosure targeting SOD1 may comprise a nucleotide sequence such as, but not limited to, the sense (passenger) sequences in Table 10 or a fragment or variant thereof. As a non-limiting example, the sense sequence used in the siRNA molecule of the present disclosure may comprises at least 15 (e.g., at least 16, 17, 18, 19, 20, 21, 22, or 23) contiguous nucleotides differing by no more than 3 (e.g., by no more than 0, 1 or 2) nucleotides from an sense sequence listed in any one of the duplexes in Table 10. As a non-limiting example, the sense sequence used in the siRNA molecule of the present disclosure is at least 80%, 81%, 82%, 83%, 84%, 85%, 86%, 87%, 88%, 89%, 90%, 91%, 92%, 93%, 94%, 95%, 96%, 97%, 98% or 99% or at least 70-80%, 70-90%, 70-95%, 70-99%, 80-90%, 80-95%, 80-99%, 90-95%, 90-99% or 95-99% identical to a sense strand nucleotide sequence in Table 10. As another non-limiting example, the sense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 10. As yet another non-limiting example, the sense sequence used in the siRNA molecule of the present disclosure comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sense strand sequences in Table 10.

In certain embodiments, the siRNA molecules of the present disclosure targeting SOD1 may comprise an antisense sequence from, or a fragment or variant thereof. As a non-limiting example, the antisense sequence and the sense sequence have at least region of complementarity ranging from 5, 6, 7, 8, 9, 10, 11, 12, 13, 14, 15, 16, 17, 18, 19, 20, or 21 nucleotides in length, or any region between (e.g., from 17-20 nucleotides in length). In some embodiments, the antisense sequence and the sense sequence are fully complementary. In some embodiments, the antisense sequence and the sense sequence have a region of complementarity ranging from 1-15, 1-16, 1-17, 1-18, 1-19, or 1-20 nucleotides in length.

In certain embodiments, the siRNA molecules of the present disclosure targeting SOD1 may comprise the sense and antisense siRNA duplex as described in Table 10. As a non-limiting example, these siRNA duplexes may be tested for in vitro inhibitory activity on endogenous SOD1 gene expression.

TABLE 10
Sense and antisense strand sequences of SOD1 dsRNA

| | | | Sense | | | Antisense | |
|---|---|---|---|---|---|---|---|
| | | | Strand | | | Strand | |
| | siRNA | Sense | Sequence | SS | | Sequence | AS |
| | Duplex | Strand | (5′-3′) | SEQ | AS | (5′-3′) | SEQ |
| Name | ID | ID | (Passenger) | ID | ID | (Guide) | ID |
| | D-2741 | 7414 | CGGAGGUCUGG | 2004 | 7415 | UUUAUAGGCCA | 2173 |
| | | | CCUAUAACdTdT | | | GACCUCCGdTdT | |
| | D-2742 | 7416 | GGAGGUCUGGC | 2005 | 7417 | UUUUAUAGGCC | 2174 |
| | | | CUAUAAACdTdT | | | AGACCUCCdTdT | |
| | D-2743 | 7418 | GAGGUCUGGCC | 2006 | 7419 | UCUUUAUAGGC | 2175 |
| | | | UAUAAAGCdTdT | | | CAGACCUCdTdT | |
| | D-2744 | 7420 | AGGUCUGGCCU | 2007 | 7421 | UACUUUAUAGG | 2176 |
| | | | AUAAAGUCdTdT | | | CCAGACCUdTdT | |
| | D-2745 | 7422 | GGUCUGGCCUA | 2008 | 7423 | UUACUUUAUAG | 2177 |
| | | | UAAAGUACdTdT | | | GCCAGACCdTdT | |
| | D-2746 | 7424 | UCUGGCCUAUA | 2009 | 7425 | UACUACUUUAU | 2178 |
| | | | AAGUAGUCdTdT | | | AGGCCAGAdTdT | |
| | D-2747 | 7426 | CUGGCCUAUAA | 2010 | 7427 | UGACUACUUUA | 2179 |
| | | | AGUAGUCCdTdT | | | UAGGCCAGdTdT | |
| | D-2748 | 7428 | UGGCCUAUAAA | 2011 | 7429 | UCGACUACUUU | 2180 |
| | | | GUAGUCGCdTdT | | | AUAGGCCAdTdT | |
| | D-2749 | 7430 | GGCCUAUAAAG | 2012 | 7431 | UGCGACUACUU | 2181 |
| | | | UAGUCGCCdTdT | | | UAUAGGCCdTdT | |
| | D-2750 | 7432 | GCCUAUAAAGU | 2013 | 7433 | UCGCGACUACU | 2182 |
| | | | AGUCGCGCdTdT | | | UUAUAGGCdTdT | |
| | D-2751 | 7434 | CCUAUAAAGUA | 2014 | 7435 | UCCGCGACUAC | 2183 |
| | | | GUCGCGGCdTdT | | | UUUAUAGGdTdT | |
| | D-2752 | 7436 | GUCGUAGUCUC | 2015 | 7437 | UGCUGCAGGAG | 2184 |
| | | | CUGCAGCCdTdT | | | ACUACGACdTdT | |
| | D-2753 | 7438 | CGUAGUCUCCU | 2016 | 7439 | UACGCUGCAGG | 2185 |
| | | | GCAGCGUCdTdT | | | AGACUACGdTdT | |
| | D-2754 | 7440 | GUAGUCUCCUG | 2017 | 7441 | UGACGCUGCAG | 2186 |
| | | | CAGCGUCCdTdT | | | GAGACUACdTdT | |
| | D-2755 | 7442 | UAGUCUCCUGC | 2018 | 7443 | UAGACGCUGCA | 2187 |
| | | | AGCGUCUCdTdT | | | GGAGACUAdTdT | |
| | D-2756 | 7444 | AUGGCGACGAA | 2019 | 7445 | UCACGGCCUUC | 2188 |
| | | | GGCCGUGCdTdT | | | GUCGCCAUdTdT | |
| | D-2757 | 7446 | CGACGAAGGCC | 2020 | 7447 | UCGCACACGGCC | 2189 |
| | | | GUGUGCGCdTdT | | | UUCGUCGdTdT | |
| | D-2758 | 7448 | GAAGGCCGUGU | 2021 | 7449 | UAGCACGCACA | 2190 |
| | | | GCGUGCUCdTdT | | | CGGCCUUCdTdT | |
| | D-2759 | 7450 | GGCCGUGUGCG | 2022 | 7451 | UUUCAGCACGC | 2191 |
| | | | UGCUGAACdTdT | | | ACACGGCCdTdT | |
| | D-2760 | 7452 | AGGGCGACGGC | 2023 | 7453 | UGCACUGGGCC | 2192 |
| | | | CCAGUGCCdTdT | | | GUCGCCCUdTdT | |
| | D-2761 | 7454 | UGCAGGGCAUC | 2024 | 7455 | UAAUUGAUGAU | 2193 |
| | | | AUCAAUUCdTdT | | | GCCCUGCAdTdT | |
| | D-2762 | 7456 | GCAGGGCAUCA | 2025 | 7457 | UAAAUUGAUGA | 2194 |
| | | | UCAAUUUCdTdT | | | UGCCCUGCdTdT | |
| | D-2763 | 7458 | AGGGCAUCAUC | 2026 | 7459 | UCGAAAUUGAU | 2195 |
| | | | AAUUUCGCdTdT | | | GAUGCCCUdTdT | |
| | D-2764 | 7460 | GGGCAUCAUCA | 2027 | 7461 | UUCGAAAUUGA | 2196 |
| | | | AUUUCGACdTdT | | | UGAUGCCCdTdT | |
| | D-2765 | 7462 | GGCAUCAUCAA | 2028 | 7463 | UCUCGAAAUUG | 2197 |
| | | | UUUCGAGCdTdT | | | AUGAUGCCdTdT | |
| | D-2766 | 7464 | GCAUCAUCAAU | 2029 | 7465 | UGCUCGAAAUU | 2198 |
| | | | UUCGAGCCdTdT | | | GAUGAUGCdTdT | |
| | D-2767 | 7466 | CAUCAUCAAUU | 2030 | 7467 | UUGCUCGAAAU | 2199 |
| | | | UCGAGCACdTdT | | | UGAUGAUGdTdT | |
| | D-2768 | 7468 | AAUUUCGAGCA | 2031 | 7469 | UUUCCUUCUGC | 2200 |
| | | | GAAGGAACdTdT | | | UCGAAAUUdTdT | |
| | D-2769 | 7470 | UUCGAGCAGAA | 2032 | 7471 | UACUUUCCUUC | 2201 |
| | | | GGAAAGUCdTdT | | | UGCUCGAAdTdT | |
| | D-2770 | 7472 | UCGAGCAGAAG | 2033 | 7473 | UUACUUUCCUU | 2202 |
| | | | GAAAGUACdTdT | | | CUGCUCGAdTdT | |
| | D-2771 | 7474 | AAGGUGUGGGG | 2034 | 7475 | UAAUGCUUCCC | 2203 |
| | | | AAGCAUUCdTdT | | | CACACCUUdTdT | |
| | D-2772 | 7476 | GGUGUGGGGAA | 2035 | 7477 | UUUAAUGCUUC | 2204 |
| | | | GCAUUAACdTdT | | | CCCACACCdTdT | |
| | D-2773 | 7478 | GACUGACUGAA | 2036 | 7479 | UGCAGGCCUUC | 2205 |
| | | | GGCCUGCCdTdT | | | AGUCAGUCdTdT | |
| | D-2774 | 7480 | CUGACUGAAGG | 2037 | 7481 | UAUGCAGGCCU | 2206 |
| | | | CCUGCAUCdTdT | | | UCAGUCAGdTdT | |
| | D-2775 | 7482 | UGACUGAAGGC | 2038 | 7483 | UCAUGCAGGCC | 2207 |
| | | | CUGCAUGCdTdT | | | UUCAGUCAdTdT | |
| | D-2776 | 7484 | UGAAGGCCUGC | 2039 | 7485 | UAAUCCAUGCA | 2208 |
| | | | AUGGAUUCdTdT | | | GGCCUUCAdTdT | |
| | D-2777 | 7486 | GAAGGCCUGCA | 2040 | 7487 | UGAAUCCAUGC | 2209 |
| | | | UGGAUUCCdTdT | | | AGGCCUUCdTdT | |
| | D-2778 | 7488 | UGCAUGGAUUC | 2041 | 7489 | UGAACAUGGAA | 2210 |
| | | | CAUGUUCCdTdT | | | UCCAUGCAdTdT | |
| | D-2779 | 7490 | CAUGGAUUCCA | 2042 | 7491 | UAUGAACAUGG | 2211 |
| | | | UGUUCAUCdTdT | | | AAUCCAUGdTdT | |
| | D-2780 | 7492 | GGAUUCCAUGU | 2043 | 7493 | UCUCAUGAACA | 2212 |
| | | | UCAUGAGCdTdT | | | UGGAAUCCdTdT | |
| | D-2781 | 7494 | UUCCAUGUUCA | 2044 | 7495 | UAAACUCAUGA | 2213 |
| | | | UGAGUUUCdTdT | | | ACAUGGAAdTdT | |
| | D-2782 | 7496 | GUUCAUGAGUU | 2045 | 7497 | UAUCUCCAAAC | 2214 |
| | | | UGGAGAUCdTdT | | | UCAUGAACdTdT | |
| | D-2783 | 7498 | UUCAUGAGUUU | 2046 | 7499 | UUAUCUCCAAA | 2215 |
| | | | GGAGAUACdTdT | | | CUCAUGAAdTdT | |
| | D-2784 | 7500 | UGAGUUUGGAG | 2047 | 7501 | UGUAUUAUCUC | 2216 |
| | | | AUAAUACCdTdT | | | CAAACUCAdTdT | |
| | D-2785 | 7502 | GAGUUUGGAGA | 2048 | 7503 | UUGUAUUAUCU | 2217 |
| | | | UAAUACACdTdT | | | CCAAACUCdTdT | |
| | D-2786 | 7504 | AGGCUGUACCA | 2049 | 7505 | UCCUGCACUGG | 2218 |
| | | | GUGCAGGCdTdT | | | UACAGCCUdTdT | |
| | D-2787 | 7506 | GGCUGUACCAG | 2050 | 7507 | UACCUGCACUG | 2219 |
| | | | UGCAGGUCdTdT | | | GUACAGCCdTdT | |
| | D-2788 | 7508 | GCAGGUCCUCA | 2051 | 7509 | UAUUAAAGUGA | 2220 |
| | | | CUUUAAUCdTdT | | | GGACCUGCdTdT | |
| | D-2789 | 7510 | CAGGUCCUCAC | 2052 | 7511 | UGAUUAAAGUG | 2221 |
| | | | UUUAAUCCdTdT | | | AGGACCUGdTdT | |
| | D-2790 | 7512 | UCACUUUAAUC | 2053 | 7513 | UGAUAGAGGAU | 2222 |
| | | | CUCUAUCCdTdT | | | UAAAGUGAdTdT | |
| | D-2791 | 7514 | CUAUCCAGAAA | 2054 | 7515 | UACCGUGUUUU | 2223 |
| | | | ACACGGUCdTdT | | | CUGGAUAGdTdT | |
| | D-2792 | 7516 | UAUCCAGAAAA | 2055 | 7517 | UCACCGUGUUU | 2224 |
| | | | CACGGUGCdTdT | | | UCUGGAUAdTdT | |
| | D-2793 | 7518 | AUCCAGAAAAC | 2056 | 7519 | UCCACCGUGUU | 2225 |
| | | | ACGGUGGCdTdT | | | UUCUGGAUdTdT | |
| | D-2794 | 7520 | CCAGAAAACAC | 2057 | 7521 | UGCCCACCGUG | 2226 |
| | | | GGUGGGCCdTdT | | | UUUUCUGGdTdT | |
| | D-2795 | 7522 | GAAAACACGGU | 2058 | 7523 | UUUGGCCCACC | 2227 |
| | | | GGGCCAACdTdT | | | GUGUUUUCdTdT | |
| | D-2796 | 7524 | AAAACACGGUG | 2059 | 7525 | UUUUGGCCCAC | 2228 |
| | | | GGCCAAACdTdT | | | CGUGUUUUdTdT | |
| | D-2797 | 7526 | CGGUGGGCCAA | 2060 | 7527 | UUCAUCCUUUG | 2229 |
| | | | AGGAUGACdTdT | | | GCCCACCGdTdT | |
| | D-2798 | 7528 | AGGAUGAAGAG | 2061 | 7529 | UCAUGCCUCUC | 2230 |
| | | | AGGCAUGCdTdT | | | UUCAUCCUdTdT | |
| | D-2799 | 7530 | AUGAAGAGAGG | 2062 | 7531 | UCAACAUGCCU | 2231 |
| | | | CAUGUUGCdTdT | | | CUCUUCAUdTdT | |
| | D-2800 | 7532 | GAGAGGCAUGU | 2063 | 7533 | UGUCUCCAACA | 2232 |
| | | | UGGAGACCdTdT | | | UGCCUCUCdTdT | |
| | D-2801 | 7534 | AGAGGCAUGUU | 2064 | 7535 | UAGUCUCCAAC | 2233 |
| | | | GGAGACUCdTdT | | | AUGCCUCUdTdT | |
| | D-2802 | 7536 | AUGUUGGAGAC | 2065 | 7537 | UUGCCCAAGUC | 2234 |
| | | | UUGGGCACdTdT | | | UCCAACAUdTdT | |
| | D-2803 | 7538 | GUUGGAGACUU | 2066 | 7539 | UAUUGCCCAAG | 2235 |
| | | | GGGCAAUCdTdT | | | UCUCCAACdTdT | |
| | D-2804 | 7540 | GGAGACUUGGG | 2067 | 7541 | UCACAUUGCCC | 2236 |
| | | | CAAUGUGCdTdT | | | AAGUCUCCdTdT | |
| | D-2805 | 7542 | GGCAAUGUGAC | 2068 | 7543 | UGUCAGCAGUC | 2237 |
| | | | UGCUGACCdTdT | | | ACAUUGCCdTdT | |
| | D-2806 | 7544 | CAAUGUGACUG | 2069 | 7545 | UUUGUCAGCAG | 2238 |
| | | | CUGACAACdTdT | | | UCACAUUGdTdT | |
| | D-2807 | 7546 | CUGACAAAGAU | 2070 | 7547 | UCCACACCAUCU | 2239 |
| | | | GGUGUGGCdTdT | | | UUGUCAGdTdT | |
| | D-2808 | 7548 | UGACAAAGAUG | 2071 | 7549 | UGCCACACCAUC | 2240 |
| | | | GUGUGGCCdTdT | | | UUUGUCAdTdT | |
| | D-2809 | 7550 | CUCAGGAGACC | 2072 | 7551 | UAUGCAAUGGU | 2241 |
| | | | AUUGCAUCdTdT | | | CUCCUGAGdTdT | |
| | D-2810 | 7552 | UCAGGAGACCA | 2073 | 7553 | UGAUGCAAUGG | 2242 |
| | | | UUGCAUCCdTdT | | | UCUCCUGAdTdT | |
| | D-2811 | 7554 | AGACCAUUGCA | 2074 | 7555 | UCCAAUGAUGC | 2243 |
| | | | UCAUUGGCdTdT | | | AAUGGUCUdTdT | |
| | D-2812 | 7556 | GACCAUUGCAU | 2075 | 7557 | UGCCAAUGAUG | 2244 |
| | | | CAUUGGCCdTdT | | | CAAUGGUCdTdT | |
| | D-2813 | 7558 | AUUGCAUCAUU | 2076 | 7559 | UUGCGGCCAAU | 2245 |
| | | | GGCCGCACdTdT | | | GAUGCAAUdTdT | |
| | D-2814 | 7560 | CAUUGGCCGCA | 2077 | 7561 | UACCAGUGUGC | 2246 |
| | | | CACUGGUCdTdT | | | GGCCAAUGdTdT | |
| | D-2815 | 7562 | CGCACACUGGU | 2078 | 7563 | UAUGGACCACC | 2247 |
| | | | GGUCCAUCdTdT | | | AGUGUGCGdTdT | |
| | D-2816 | 7564 | CACACUGGUGG | 2079 | 7565 | UUCAUGGACCA | 2248 |
| | | | UCCAUGACdTdT | | | CCAGUGUGdTdT | |
| | D-2817 | 7566 | ACACUGGUGGU | 2080 | 7567 | UUUCAUGGACC | 2249 |
| | | | CCAUGAACdTdT | | | ACCAGUGUdTdT | |
| | D-2818 | 7568 | UGGUGGUCCAU | 2081 | 7569 | UCUUUUUCAUG | 2250 |
| | | | GAAAAAGCdTdT | | | GACCACCAdTdT | |
| | D-2819 | 7570 | UGGUCCAUGAA | 2082 | 7571 | UCUGCUUUUUC | 2251 |
| | | | AAAGCAGCdTdT | | | AUGGACCAdTdT | |
| | D-2820 | 7572 | AAAGCAGAUGA | 2083 | 7573 | UGCCCAAGUCA | 2252 |
| | | | CUUGGGCCdTdT | | | UCUGCUUUdTdT | |
| | D-2821 | 7574 | GCAGAUGACUU | 2084 | 7575 | UUUUGCCCAAG | 2253 |
| | | | GGGCAAACdTdT | | | UCAUCUGCdTdT | |
| | D-2822 | 7576 | AUGACUUGGGC | 2085 | 7577 | UCACCUUUGCCC | 2254 |
| | | | AAAGGUGCdTdT | | | AAGUCAUdTdT | |
| | D-2823 | 7578 | UGACUUGGGCA | 2086 | 7579 | UCCACCUUUGCC | 2255 |
| | | | AAGGUGGCdTdT | | | CAAGUCAdTdT | |
| | D-2824 | 7580 | GACUUGGGCAA | 2087 | 7581 | UUCCACCUUUG | 2256 |
| | | | AGGUGGACdTdT | | | CCCAAGUCdTdT | |
| | D-2825 | 7582 | GUACAAAGACA | 2088 | 7583 | UCGUUUCCUGU | 2257 |
| | | | GGAAACGCdTdT | | | CUUUGUACdTdT | |
| | D-2826 | 7584 | ACAAAGACAGG | 2089 | 7585 | UAGCGUUUCCU | 2258 |
| | | | AAACGCUCdTdT | | | GUCUUUGUdTdT | |
| | D-2827 | 7586 | CAAAGACAGGA | 2090 | 7587 | UCAGCGUUUCC | 2259 |
| | | | AACGCUGCdTdT | | | UGUCUUUGdTdT | |
| | D-2828 | 7588 | AGGAAACGCUG | 2091 | 7589 | UCGACUUCCAG | 2260 |
| | | | GAAGUCGCdTdT | | | CGUUUCCUdTdT | |
| | D-2829 | 7590 | GUCGUUUGGCU | 2092 | 7591 | UCACCACAAGCC | 2261 |
| | | | UGUGGUGCdTdT | | | AAACGACdTdT | |
| | D-2830 | 7592 | UCGUUUGGCUU | 2093 | 7593 | UACACCACAAG | 2262 |
| | | | GUGGUGUCdTdT | | | CCAAACGAdTdT | |
| | D-2831 | 7594 | CGUUUGGCUUG | 2094 | 7595 | UUACACCACAA | 2263 |
| | | | UGGUGUACdTdT | | | GCCAAACGdTdT | |
| | D-2832 | 7596 | GUUUGGCUUGU | 2095 | 7597 | UUUACACCACA | 2264 |
| | | | GGUGUAACdTdT | | | AGCCAAACdTdT | |
| | D-2833 | 7598 | UUGGCUUGUGG | 2096 | 7599 | UAAUUACACCA | 2265 |
| | | | UGUAAUUCdTdT | | | CAAGCCAAdTdT | |
| | D-2834 | 7600 | GGCUUGUGGUG | 2097 | 7601 | UCCAAUUACAC | 2266 |
| | | | UAAUUGGCdTdT | | | CACAAGCCdTdT | |
| | D-2835 | 7602 | GCUUGUGGUGU | 2098 | 7603 | UCCCAAUUACA | 2267 |
| | | | AAUUGGGCdTdT | | | CCACAAGCdTdT | |
| | D-2836 | 7604 | CUUGUGGUGUA | 2099 | 7605 | UUCCCAAUUAC | 2268 |
| | | | AUUGGGACdTdT | | | ACCACAAGdTdT | |
| | D-2837 | 7606 | UGUGGUGUAAU | 2100 | 7607 | UGAUCCCAAUU | 2269 |
| | | | UGGGAUCCdTdT | | | ACACCACAdTdT | |
| | D-2838 | 7608 | GUGGUGUAAUU | 2101 | 7609 | UCGAUCCCAAU | 2270 |
| | | | GGGAUCGCdTdT | | | UACACCACdTdT | |
| | D-2839 | 7610 | UGGUGUAAUUG | 2102 | 7611 | UGCGAUCCCAA | 2271 |
| | | | GGAUCGCCdTdT | | | UUACACCAdTdT | |
| | D-2840 | 7612 | GUAAUUGGGAU | 2103 | 7613 | UUUGGGCGAUC | 2272 |
| | | | CGCCCAACdTdT | | | CCAAUUACdTdT | |
| | D-2841 | 7614 | UAAUUGGGAUC | 2104 | 7615 | UAUUGGGCGAU | 2273 |
| | | | GCCCAAUCdTdT | | | CCCAAUUAdTdT | |
| | D-2842 | 7616 | AAUUGGGAUCG | 2105 | 7617 | UUAUUGGGCGA | 2274 |
| | | | CCCAAUACdTdT | | | UCCCAAUUdTdT | |
| | D-2843 | 7618 | AUUGGGAUCGC | 2106 | 7619 | UUUAUUGGGCG | 2275 |
| | | | CCAAUAACdTdT | | | AUCCCAAUdTdT | |
| | D-2844 | 7620 | UUGGGAUCGCC | 2107 | 7621 | UUUUAUUGGGC | 2276 |
| | | | CAAUAAACdTdT | | | GAUCCCAAdTdT | |
| | D-2845 | 7622 | UGGGAUCGCCC | 2108 | 7623 | UGUUUAUUGGG | 2277 |
| | | | AAUAAACCdTdT | | | CGAUCCCAdTdT | |
| | D-2846 | 7624 | GGGAUCGCCCA | 2109 | 7625 | UUGUUUAUUGG | 2278 |
| | | | AUAAACACdTdT | | | GCGAUCCCdTdT | |
| | D-2847 | 7626 | AUCGCCCAAUA | 2110 | 7627 | UGAAUGUUUAU | 2279 |
| | | | AACAUUCCdTdT | | | UGGGCGAUdTdT | |
| | D-2848 | 7628 | CCAAUAAACAU | 2111 | 7629 | UCAAGGGAAUG | 2280 |
| | | | UCCCUUGCdTdT | | | UUUAUUGGdTdT | |
| | D-2849 | 7630 | CAAUAAACAUU | 2112 | 7631 | UCCAAGGGAAU | 2281 |
| | | | CCCUUGGCdTdT | | | GUUUAUUGdTdT | |
| | D-2850 | 7632 | AAUAAACAUUC | 2113 | 7633 | UUCCAAGGGAA | 2282 |
| | | | CCUUGGACdTdT | | | UGUUUAUUdTdT | |
| | D-2851 | 7634 | AUAAACAUUCC | 2114 | 7635 | UAUCCAAGGGA | 2283 |
| | | | CUUGGAUCdTdT | | | AUGUUUAUdTdT | |
| | D-2852 | 7636 | UAAACAUUCCC | 2115 | 7637 | UCAUCCAAGGG | 2284 |
| | | | UUGGAUGCdTdT | | | AAUGUUUAdTdT | |
| | D-2853 | 7638 | AAACAUUCCCU | 2116 | 7639 | UACAUCCAAGG | 2285 |
| | | | UGGAUGUCdTdT | | | GAAUGUUUdTdT | |
| | D-2854 | 7640 | AACAUUCCCUU | 2117 | 7641 | UUACAUCCAAG | 2286 |
| | | | GGAUGUACdTdT | | | GGAAUGUUdTdT | |
| | D-2855 | 7642 | AUUCCCUUGGA | 2118 | 7643 | UGACUACAUCC | 2287 |
| | | | UGUAGUCCdTdT | | | AAGGGAAUdTdT | |
| | D-2856 | 7644 | CUUGGAUGUAG | 2119 | 7645 | UCCUCAGACUA | 2288 |
| | | | UCUGAGGCdTdT | | | CAUCCAAGdTdT | |
| | D-2857 | 7646 | CUGAGGCCCCU | 2120 | 7647 | UUGAGUUAAGG | 2289 |
| | | | UAACUCACdTdT | | | GGCCUCAGdTdT | |
| | D-2858 | 7648 | GAGGCCCCUUA | 2121 | 7649 | UGAUGAGUUAA | 2290 |
| | | | ACUCAUCCdTdT | | | GGGGCCUCdTdT | |
| | D-2859 | 7650 | AGGCCCCUUAA | 2122 | 7651 | UAGAUGAGUUA | 2291 |
| | | | CUCAUCUCdTdT | | | AGGGGCCUdTdT | |
| | D-2860 | 7652 | CCCCUUAACUCA | 2123 | 7653 | UAACAGAUGAG | 2292 |
| | | | UCUGUUCdTdT | | | UUAAGGGGdTdT | |
| | D-2861 | 7654 | CCCUUAACUCA | 2124 | 7655 | UUAACAGAUGA | 2293 |
| | | | UCUGUUACdTdT | | | GUUAAGGGdTdT | |
| | D-2862 | 7656 | CCUUAACUCAU | 2125 | 7657 | UAUAACAGAUG | 2294 |
| | | | CUGUUAUCdTdT | | | AGUUAAGGdTdT | |
| | D-2863 | 7658 | CUUAACUCAUC | 2126 | 7659 | UGAUAACAGAU | 2295 |
| | | | UGUUAUCCdTdT | | | GAGUUAAGdTdT | |
| | D-2864 | 7660 | UUAACUCAUCU | 2127 | 7661 | UGGAUAACAGA | 2296 |
| | | | GUUAUCCCdTdT | | | UGAGUUAAdTdT | |
| | D-2865 | 7662 | UAACUCAUCUG | 2128 | 7663 | UAGGAUAACAG | 2297 |
| | | | UUAUCCUCdTdT | | | AUGAGUUAdTdT | |
| | D-2866 | 7664 | AACUCAUCUGU | 2129 | 7665 | UCAGGAUAACA | 2298 |
| | | | UAUCCUGCdTdT | | | GAUGAGUUdTdT | |
| | D-2867 | 7666 | GUUAUCCUGCU | 2130 | 7667 | UUACAGCUAGC | 2299 |
| | | | AGCUGUACdTdT | | | AGGAUAACdTdT | |
| | D-2868 | 7668 | CUGCUAGCUGU | 2131 | 7669 | UCAUUUCUACA | 2300 |
| | | | AGAAAUGCdTdT | | | GCUAGCAGdTdT | |
| | D-2869 | 7670 | UGCUAGCUGUA | 2132 | 7671 | UACAUUUCUAC | 2301 |
| | | | GAAAUGUCdTdT | | | AGCUAGCAdTdT | |
| | D-2870 | 7672 | GCUGUAGAAAU | 2133 | 7673 | UAGGAUACAUU | 2302 |
| | | | GUAUCCUCdTdT | | | UCUACAGCdTdT | |
| | D-2871 | 7674 | CUGUAGAAAUG | 2134 | 7675 | UCAGGAUACAU | 2303 |
| | | | UAUCCUGCdTdT | | | UUCUACAGdTdT | |
| | D-2872 | 7676 | UGUAGAAAUGU | 2135 | 7677 | UUCAGGAUACA | 2304 |
| | | | AUCCUGACdTdT | | | UUUCUACAdTdT | |
| | D-2873 | 7678 | GUAGAAAUGUA | 2136 | 7679 | UAUCAGGAUAC | 2305 |
| | | | UCCUGAUCdTdT | | | AUUUCUACdTdT | |
| | D-2874 | 7680 | AAAUGUAUCCU | 2137 | 7681 | UGUUUAUCAGG | 2306 |
| | | | GAUAAACCdTdT | | | AUACAUUUdTdT | |
| | D-2875 | 7682 | GUAUCCUGAUA | 2138 | 7683 | UUAAUGUUUAU | 2307 |
| | | | AACAUUACdTdT | | | CAGGAUACdTdT | |
| | D-2876 | 7684 | UUAAACACUGU | 2139 | 7685 | UUAAGAUUACA | 2308 |
| | | | AAUCUUACdTdT | | | GUGUUUAAdTdT | |
| | D-2877 | 7686 | ACUGUAAUCUU | 2140 | 7687 | UCACUUUUAAG | 2309 |
| | | | AAAAGUGCdTdT | | | AUUACAGUdTdT | |
| | D-2878 | 7688 | CUGUAAUCUUA | 2141 | 7689 | UACACUUUUAA | 2310 |
| | | | AAAGUGUCdTdT | | | GAUUACAGdTdT | |
| | D-2879 | 7690 | UGUAAUCUUAA | 2142 | 7691 | UUACACUUUUA | 2311 |
| | | | AAGUGUACdTdT | | | AGAUUACAdTdT | |
| | D-2880 | 7692 | GUAAUCUUAAA | 2143 | 7693 | UUUACACUUUU | 2312 |
| | | | AGUGUAACdTdT | | | AAGAUUACdTdT | |
| | D-2881 | 7694 | CUUAAAAGUGU | 2144 | 7695 | UCACAAUUACA | 2313 |
| | | | AAUUGUGCdTdT | | | CUUUUAAGdTdT | |
| | D-2882 | 7696 | UACCUGUAGUG | 2145 | 7697 | UAGUUUCUCAC | 2314 |
| | | | AGAAACUCdTdT | | | UACAGGUAdTdT | |
| | D-2883 | 7698 | UUAUGAUCACU | 2146 | 7699 | UUCUUCCAAGU | 2315 |
| | | | UGGAAGACdTdT | | | GAUCAUAAdTdT | |
| | D-2884 | 7700 | AUGAUCACUUG | 2147 | 7701 | UAAUCUUCCAA | 2316 |
| | | | GAAGAUUCdTdT | | | GUGAUCAUdTdT | |
| | D-2885 | 7702 | AUCACUUGGAA | 2148 | 7703 | UACAAAUCUUC | 2317 |
| | | | GAUUUGUCdTdT | | | CAAGUGAUdTdT | |
| | D-2886 | 7704 | UGGAAGAUUUG | 2149 | 7705 | UAACUAUACAA | 2318 |
| | | | UAUAGUUCdTdT | | | AUCUUCCAdTdT | |
| | D-2887 | 7706 | UAUAAAACUCA | 2150 | 7707 | UUUUUAACUGA | 2319 |
| | | | GUUAAAACdTdT | | | GUUUUAUAdTdT | |
| | D-2888 | 7708 | AAACUCAGUUA | 2151 | 7709 | UGACAUUUUAA | 2320 |
| | | | AAAUGUCCdTdT | | | CUGAGUUUdTdT | |
| | D-2889 | 7710 | GUCUGUUUCAA | 2152 | 7711 | UCAGGUCAUUG | 2321 |
| | | | UGACCUGCdTdT | | | AAACAGACdTdT | |
| | D-2890 | 7712 | AUGACCUGUAU | 2153 | 7713 | UUGGCAAAAUA | 2322 |
| | | | UUUGCCACdTdT | | | CAGGUCAUdTdT | |
| | D-2891 | 7714 | ACCUGUAUUUU | 2154 | 7715 | UGUCUGGCAAA | 2323 |
| | | | GCCAGACCdTdT | | | AUACAGGUdTdT | |
| | D-2892 | 7716 | CCUGUAUUUUG | 2155 | 7717 | UAGUCUGGCAA | 2324 |
| | | | CCAGACUCdTdT | | | AAUACAGGdTdT | |
| | D-2893 | 7718 | UAAAUCACAGA | 2156 | 7719 | UAUACCCAUCU | 2325 |
| | | | UGGGUAUCdTdT | | | GUGAUUUAdTdT | |
| | D-2894 | 7720 | AUCACAGAUGG | 2157 | 7721 | UUUAAUACCCA | 2326 |
| | | | GUAUUAACdTdT | | | UCUGUGAUdTdT | |
| | D-2895 | 7722 | UCACAGAUGGG | 2158 | 7723 | UUUUAAUACCC | 2327 |
| | | | UAUUAAACdTdT | | | AUCUGUGAdTdT | |
| | D-2896 | 7724 | ACAGAUGGGUA | 2159 | 7725 | UAGUUUAAUAC | 2328 |
| | | | UUAAACUCdTdT | | | CCAUCUGUdTdT | |
| | D-2897 | 7726 | CAGAUGGGUAU | 2160 | 7727 | UAAGUUUAAUA | 2329 |
| | | | UAAACUUCdTdT | | | CCCAUCUGdTdT | |
| | D-2898 | 7728 | AGAUGGGUAUU | 2161 | 7729 | UCAAGUUUAAU | 2330 |
| | | | AAACUUGCdTdT | | | ACCCAUCUdTdT | |
| | D-2899 | 7730 | AUGGGUAUUAA | 2162 | 7731 | UGACAAGUUUA | 2331 |
| | | | ACUUGUCCdTdT | | | AUACCCAUdTdT | |
| | D-2900 | 7732 | UAAACUUGUCA | 2163 | 7733 | UGAAAUUCUGA | 2332 |
| | | | GAAUUUCCdTdT | | | CAAGUUUAdTdT | |
| | D-2901 | 7734 | UCAUUCAAGCC | 2164 | 7735 | UAUUCACAGGC | 2333 |
| | | | UGUGAAUCdTdT | | | UUGAAUGAdTdT | |
| | D-2902 | 7736 | CAUUCAAGCCU | 2165 | 7737 | UUAUUCACAGG | 2334 |
| | | | GUGAAUACdTdT | | | CUUGAAUGdTdT | |
| | D-2903 | 7738 | AAUAAAAACCC | 2166 | 7739 | UCCAUACAGGG | 2335 |
| | | | UGUAUGGCdTdT | | | UUUUUAUUdTdT | |
| | D-2904 | 7740 | AUAAAAACCCU | 2167 | 7741 | UGCCAUACAGG | 2336 |
| | | | GUAUGGCCdTdT | | | GUUUUUAUdTdT | |
| | D-2905 | 7742 | AACCCUGUAUG | 2168 | 7743 | UUAAGUGCCAU | 2337 |
| | | | GCACUUACdTdT | | | ACAGGGUUdTdT | |
| | D-2906 | 7744 | ACCCUGUAUGG | 2169 | 7745 | UAUAAGUGCCA | 2338 |
| | | | CACUUAUCdTdT | | | UACAGGGUdTdT | |
| | D-2907 | 7746 | GAGGCUAUUAA | 2170 | 7747 | UGAUUCUUUUA | 2339 |
| | | | AAGAAUCCdTdT | | | AUAGCCUCdTdT | |
| | D-2908 | 7748 | AAAGAAUCCAA | 2171 | 7749 | UUUUGAAUUUG | 2340 |
| | | | AUUCAAACdTdT | | | GAUUCUUUdTdT | |
| | D-2909 | 7750 | GAAUCCAAAUU | 2172 | 7751 | UUAGUUUGAAU | 2341 |
| | | | CAAACUACdTdT | | | UUGGAUUCdTdT | |
| VOYpre- | D-2910 | 7752 | CAAUGUGACUG | 2342 | 7753 | UUUGUCAGCAG | 2343 |
| 001_D- | | | CUGACAACCC | | | UCACAUUGUU | |
| 2806_Starting | | | | | | | |
| construct (18 | | | | | | | |
| native | | | | | | | |
| nucleotides and | | | | | | | |
| position 19 is | | | | | | | |
| C; 3′ terminal | | | | | | | |
| CC | | | | | | | |
| dinucleotide) | | | | | | | |
| VOYpre- | D-2911 | 7754 | CAAUGUGACUG | 2344 | 7753 | UUUGUCAGCAG | 2343 |
| 002_D- | | | CUGACAAUCC | | | UCACAUUGUU | |
| 2806_p19MMU | | | | | | | |
| (position 19 U | | | | | | | |
| to form | | | | | | | |
| mismatch) | | | | | | | |
| VOYpre- | D-2912 | 7755 | CAAUGUGACUG | 2345 | 7753 | UUUGUCAGCAG | 2343 |
| 003_D- | | | CUGACAAGCC | | | UCACAUUGUU | |
| 2806_p19GUpair | | | | | | | |
| (position 19 | | | | | | | |
| is G to form GU | | | | | | | |
| pair) | | | | | | | |
| VOYpre- | D-2913 | 7756 | CAAUGUGACUG | 2346 | 7753 | UUUGUCAGCAG | 2343 |
| 004_D- | | | CUGACAAACC | | | UCACAUUGUU | |
| 2806_p19AUpair | | | | | | | |
| (position 19 | | | | | | | |
| is A to form AU | | | | | | | |
| pair) | | | | | | | |
| VOYpre- | D-2914 | 7757 | CAAUGUGACAG | 2347 | 7753 | UUUGUCAGCAG | 2343 |
| 005_D- | | | CUGACAAACC | | | UCACAUUGUU | |
| 2806_CMM | | | | | | | |
| (central | | | | | | | |
| mismatch) | | | | | | | |
| VOYpre- | D-2915 | 7758 | CAAUGUGACUG | 2348 | 7753 | UUUGUCAGCAG | 2343 |
| 006_D- | | | CUGACAACC | | | UCACAUUGUU | |
| 2806_p19DEL | | | | | | | |
| (position 19 | | | | | | | |
| deleted) | | | | | | | |
| VOYpre- | D-2916 | 7759 | CAAUGUGACUG | 2349 | 7753 | UUUGUCAGCAG | 2343 |
| 007_D- | | | CUGACAAUCCC | | | UCACAUUGUU | |
| 2806_p19ADD | | | | | | | |
| (nucleotide | | | | | | | |
| added at | | | | | | | |
| position 19; | | | | | | | |
| addition is U; | | | | | | | |
| keep C and | | | | | | | |
| terminal CC | | | | | | | |
| dinucleotide) | | | | | | | |
| VOYpre- | D-2917 | 7752 | CAAUGUGACUG | 2342 | 7753 | UUUGUCAGCAG | 2343 |
| 008_D- | | | CUGACAACCC | | | UCACAUUGUU | |
| 2806_Uloop | | | | | | | |
| VOYpre- | D-2918 | 7752 | CAAUGUGACUG | 2342 | 7753 | UUUGUCAGCAG | 2343 |
| 009_D- | | | CUGACAACCC | | | UCACAUUGUU | |
| 2806_AUloop | | | | | | | |
| VOYpre- | D-2919 | 7760 | CAAUGUGACUG | 2350 | 7753 | UUUGUCAGCAG | 2343 |
| 010_D- | | | CUGACAACAC | | | UCACAUUGUU | |
| 2806_mir-22- | | | | | | | |
| loop | | | | | | | |
| VOYmiR- | D-2923 | 7752 | CAAUGUGACUG | 2342 | 7753 | UUUGUCAGCAG | 2343 |
| 101_pre-001 | | | CUGACAACCC | | | UCACAUUGUU | |
| hsa-mir-155; D- | | | | | | | |
| 2806 | | | | | | | |
| VOYmiR- | D-2924 | 7752 | CAAUGUGACUG | 2342 | 7753 | UUUGUCAGCAG | 2343 |
| 102_pre-001 | | | CUGACAACCC | | | UCACAUUGUU | |
| Engineered; D- | | | | | | | |
| 2806; let-7b | | | | | | | |
| stem | | | | | | | |
| VOYmiR- | D-2925 | 7754 | CAAUGUGACUG | 2344 | 7753 | UUUGUCAGCAG | 2343 |
| 103_pre-002 | | | CUGACAAUCC | | | UCACAUUGUU | |
| Engineered; D- | | | | | | | |
| 2806_p19MMU; | | | | | | | |
| let-7b stem | | | | | | | |
| VOYmiR- | D-2926 | 7755 | CAAUGUGACUG | 2345 | 7753 | UUUGUCAGCAG | 2343 |
| 104_pre-003 | | | CUGACAAGCC | | | UCACAUUGUU | |
| Engineered; D- | | | | | | | |
| 2806_ | | | | | | | |
| p19GUpair; | | | | | | | |
| let-7b stem | | | | | | | |
| VOYmiR- | D-2927 | 7756 | CAAUGUGACUG | 2346 | 7753 | UUUGUCAGCAG | 2343 |
| 105_pre-004 | | | CUGACAAACC | | | UCACAUUGUU | |
| Engineered; D- | | | | | | | |
| 2806_p19AUpair; | | | | | | | |
| let-7b stem | | | | | | | |
| VOYmiR- | D-2928 | 7757 | CAAUGUGACAG | 2347 | 7753 | UUUGUCAGCAG | 2343 |
| 106_pre-005 | | | CUGACAAACC | | | UCACAUUGUU | |
| Engineered; D- | | | | | | | |
| 2806_CMM; | | | | | | | |
| let-7b stem | | | | | | | |
| VOYmiR- | D-2929 | 7758 | CAAUGUGACUG | 2348 | 7753 | UUUGUCAGCAG | 2343 |
| 107_pre-006 | | | CUGACAACC | | | UCACAUUGUU | |
| Engineered; D- | | | | | | | |
| 2806_p19DEL; | | | | | | | |
| let-7b stem | | | | | | | |
| VOYmiR- | D-2930 | 7765 | CAAUGUGACUG | 2355 | 7753 | UUUGUCAGCAG | 2343 |
| 108_pre-007 | | | CUGACAAUCCC | | | UCACAUUGUU | |
| Engineered; D- | | | | | | | |
| 2806_p19ADD; | | | | | | | |
| let-7b stem | | | | | | | |
| VOYmiR- | D-2931 | 7752 | CAAUGUGACUG | 2342 | 7753 | UUUGUCAGCAG | 2343 |
| 109_pre-008 | | | CUGACAACCC | | | UCACAUUGUU | |
| Engineered; D- | | | | | | | |
| 2806_Uloop; | | | | | | | |
| let-7b stem | | | | | | | |
| VOYmiR- | D-2932 | 7752 | CAAUGUGACUG | 2342 | 7753 | UUUGUCAGCAG | 2343 |
| 110_pre-009 | | | CUGACAACCC | | | UCACAUUGUU | |
| Engineered; D- | | | | | | | |
| 2806_AUloop; | | | | | | | |
| let-7b stem | | | | | | | |
| VOYmiR- | D-2933 | 7760 | CAAUGUGACUG | 2350 | 7753 | UUUGUCAGCAG | 2343 |
| 111_pre-010 | | | CUGACAACAC | | | UCACAUUGUU | |
| Engineered; D- | | | | | | | |
| 2806_mir-22- | | | | | | | |
| loop; let-7b | | | | | | | |
| stem | | | | | | | |
| VOYmiR- | D-2934 | 7752 | CAAUGUGACUG | 2342 | 7753 | UUUGUCAGCAG | 2343 |
| 112_pre-001 | | | CUGACAACCC | | | UCACAUUGUU | |
| Engineered; | | | | | | | |
| PD; D-2806; | | | | | | | |
| let-7b basal- | | | | | | | |
| stem | | | | | | | |
| instability | | | | | | | |
| VOYmiR- | D-2935 | 7754 | CAAUGUGACUG | 2344 | 7753 | UUUGUCAGCAG | 2343 |
| 113_pre-002 | | | CUGACAAUCC | | | UCACAUUGUU | |
| Engineered; D- | | | | | | | |
| 2806_p19MMU; | | | | | | | |
| let-7b basal- | | | | | | | |
| stem | | | | | | | |
| instability | | | | | | | |
| VOYmiR- | D-2936 | 7757 | CAAUGUGACAG | 2347 | 7753 | UUUGUCAGCAG | 2343 |
| 114_pre-005 | | | CUGACAAACC | | | UCACAUUGUU | |
| Engineered; D- | | | | | | | |
| 2806_CMM; | | | | | | | |
| let-7b basal- | | | | | | | |
| stem | | | | | | | |
| instability | | | | | | | |
| VOYmiR- | D-2937 | 7760 | CAAUGUGACUG | 2350 | 7753 | UUUGUCAGCAG | 2343 |
| 115_pre-010 | | | CUGACAACAC | | | UCACAUUGUU | |
| Engineered; D- | | | | | | | |
| 2806_mir-22- | | | | | | | |
| loop; let-7b | | | | | | | |
| basal-stem | | | | | | | |
| instability | | | | | | | |
| VOYmiR- | D-2938 | 7755 | CAAUGUGACUG | 2345 | 7753 | UUUGUCAGCAG | 2343 |
| 116_pre-003 | | | CUGACAAGCC | | | UCACAUUGUU | |
| Engineered; D- | | | | | | | |
| 2806_p19GUpair; | | | | | | | |
| let-7b basal- | | | | | | | |
| stem | | | | | | | |
| instability | | | | | | | |
| VOYmiR- | D-2939 | 7766 | CGACGAAGGCC | 2356 | 7767 | UCGCACACGGCC | 2357 |
| 117_pre-001 | | | GUGUGCGCCC | | | UUCGUCGUU | |
| Engineered; D- | | | | | | | |
| 2757; let-7b | | | | | | | |
| stem | | | | | | | |
| VOYmiR- | D-2940 | 7768 | UGACUUGGGCA | 2358 | 7769 | UCCACCUUUGCC | 2359 |
| 118_pre-001 | | | AAGGUGGCCC | | | CAAGUCAUU | |
| Engineered; D- | | | | | | | |
| 2823; let-7b | | | | | | | |
| stem | | | | | | | |
| VOYmiR- | D-2941 | 7770 | AACUCAUCUGU | 2360 | 7771 | UCAGGAUAACA | 2361 |
| 119_pre-001 | | | UAUCCUGCCC | | | GAUGAGUUUU | |
| Engineered; D- | | | | | | | |
| 2866; let-7b | | | | | | | |
| stem | | | | | | | |
| VOYpre- | D-2920 | 7761 | UUUGUCAGCAG | 2351 | 7762 | CAAUGUGACUG | 2352 |
| 011_D-2806_ | | | UCACAUUGUC | | | CUGACAAAUC | |
| passenger | | | | | | | |
| -guide strand | | | | | | | |
| swap with | | | | | | | |
| terminal 3′ C | | | | | | | |
| on passenger | | | | | | | |
| strand | | | | | | | |
| VOYpre- | D-2921 | 7761 | UUUGUCAGCAG | 2351 | 7763 | CAAUGUGACUG | 2353 |
| 012_D-2806 | | | UCACAUUGUC | | | CUGACAAUUC | |
| passenger-guide | | | | | | | |
| strand swap | | | | | | | |
| with | | | | | | | |
| terminal 3′ | | | | | | | |
| C on passenger | | | | | | | |
| strand | | | | | | | |
| VOYpre- | D-2922 | 7764 | UUUGUCAGCAG | 2354 | 7762 | CAAUGUGACUG | 2352 |
| 013_D-2806 | | | UCACAUUGAC | | | CUGACAAAUC | |
| passenger-guide | | | | | | | |
| strand swap | | | | | | | |
| with terminal 3′ | | | | | | | |
| C on passenger | | | | | | | |
| strand | | | | | | | |
| VOYmiR-127 | D-2942 | 7752 | CAAUGUGACUG | 2342 | 7753 | UUUGUCAGCAG | 2343 |
| | | | CUGACAACCC | | | UCACAUUGUU | |
| VOYmiR- | D-2943 | 7772 | CCCCUUAACUCA | 2362 | 7773 | UAACAGAUGAG | 2363 |
| 102.860 | | | UCUGUUCCC | | | UUAAGGGGUU | |
| VOYmiR | D-2944 | 7774 | CCCUUAACUCA | 2364 | 7775 | UUAACAGAUGA | 2365 |
| 102.861 | | | UCUGUUACCC | | | GUUAAGGGUU | |
| VOYmiR- | D-2945 | 7776 | AACUCAUCUGU | 2366 | 7771 | UCAGGAUAACA | 2361 |
| 102.866 | | | UAUCUUGCCC | | | GAUGAGUUUU | |
| VOYmiR- | D-2946 | 7777 | GCUGUGGAAAU | 2367 | 7778 | UAGGAUACAUU | 2368 |
| 102.870 | | | GUAUCUUCCC | | | UCUACAGCUU | |
| VOYmiR- | D-2947 | 7779 | UGACUUGGGCA | 2369 | 7769 | UCCACCUUUGCC | 2359 |
| 102.823 | | | AAGGUGAGCC | | | CAAGUCAUU | |
| VOYmiR- | D-2948 | 7780 | CCCCUUAACUCA | 2370 | 7773 | UAACAGAUGAG | 2363 |
| 104.860 | | | UCUGUUGCC | | | UUAAGGGGUU | |
| VOYmiR- | D-2949 | 7781 | CCCUUAACUCA | 2371 | 7775 | UUAACAGAUGA | 2365 |
| 104.861 | | | UCUGUUAGCC | | | GUUAAGGGUU | |
| VOYmiR- | D-2950 | 7782 | AACUCAUCUGU | 2372 | 7771 | UCAGGAUAACA | 2361 |
| 104.866 | | | UAUCUUAGCO | | | GAUGAGUUUU | |
| VOYmiR- | D-2951 | 7783 | GCUGUGGAAAU | 2373 | 7778 | UAGGAUACAUU | 2368 |
| 104.870 | | | GUAUCUUGCC | | | UCUACAGCUU | |
| VOYmiR- | D-2952 | 7784 | UGACUUGGGCA | 2374 | 7769 | UCCACCUUUGCC | 2359 |
| 104.823 | | | AAGGUAGGCC | | | CAAGUCAUU | |
| VOYmiR- | D-2953 | 7772 | CCCCUUAACUCA | 2362 | 7773 | UAACAGAUGAG | 2363 |
| 109.860 | | | UCUGUUCCC | | | UUAAGGGGUU | |
| VOYmiR- | D-2954 | 7774 | CCCUUAACUCA | 2364 | 7775 | UUAACAGAUGA | 2365 |
| 104.861 | | | UCUGUUACCC | | | GUUAAGGGUU | |
| VOYmiR- | D-2955 | 7776 | AACUCAUCUGU | 2366 | 7771 | UCAGGAUAACA | 2361 |
| 104.866 | | | UAUCUUGCCC | | | GAUGAGUUUU | |
| VOYmiR- | D-2956 | 7777 | GCUGUGGAAAU | 2367 | 7778 | UAGGAUACAUU | 2368 |
| 109.870 | | | GUAUCUUCCC | | | UCUACAGCUU | |
| VOYmiR- | D-2957 | 7779 | UGACUUGGGCA | 2369 | 7769 | UCCACCUUUGCC | 2359 |
| 109.823 | | | AAGGUGAGCC | | | CAAGUCAUU | |
| VOYmiR- | D-2958 | 7785 | CCCCUUAACACA | 2375 | 7773 | UAACAGAUGAG | 2363 |
| 114.860 | | | UCUGUUACC | | | UUAAGGGGUU | |
| VOYmiR- | D-2959 | 7786 | CCCUUAACUGA | 2376 | 7775 | UUAACAGAUGA | 2365 |
| 114.861 | | | UCUGUUAACC | | | GUUAAGGGUU | |
| VOYmiR- | D-2960 | 7787 | AACUCAUCUCU | 2377 | 7771 | UCAGGAUAACA | 2361 |
| 114.866 | | | UAUCUUGCCC | | | GAUGAGUUUU | |
| VOYmiR- | D-2961 | 7788 | GCUGUGGAAUU | 2378 | 7778 | UAGGAUACAUU | 2368 |
| 114.870 | | | GUAUCUUGCC | | | UCUACAGCUU | |
| VOYmiR- | D-2962 | 7789 | UGACUUGGGGA | 2379 | 7769 | UCCACCUUUGCC | 2359 |
| 114.823 | | | AAGGUGAGCC | | | CAAGUCAUU | |
| VOYmiR- | D-2963 | 7780 | CCCCUUAACUCA | 2370 | 7773 | UAACAGAUGAG | 2363 |
| 116.860 | | | UCUGUUGCC | | | UUAAGGGGUU | |
| VOYmiR- | D-2964 | 7781 | CCCUUAACUCA | 2371 | 7775 | UUAACAGAUGA | 2365 |
| 116.861 | | | UCUGUUAGCC | | | GUUAAGGGUU | |
| VOYmiR- | D-2965 | 7790 | AACUCAUCUGU | 2380 | 7771 | UCAGGAUAACA | 2361 |
| 116.866 | | | UAUCUUGGCC | | | GAUGAGUUUU | |
| VOYmiR- | D-2966 | 7783 | GCUGUGGAAAU | 2373 | 7778 | UAGGAUACAUU | 2368 |
| 116.870 | | | GUAUCUUGCC | | | UCUACAGCUU | |
| VOYmiR- | D-2967 | 7784 | UGACUUGGGCA | 2374 | 7769 | UCCACCUUUGCC | 2359 |
| 116.823 | | | AAGGUAGGCC | | | CAAGUCAUU | |
| VoymiR- | D-2968 | 7791 | CCCCUUAACUCA | 2381 | 7773 | UAACAGAUGAG | 2363 |
| 127.860 | | | UUUGUUCCC | | | UUAAGGGGUU | |
| VoymiR- | D-2969 | 7774 | CCCUUAACUCA | 2364 | 7775 | UUAACAGAUGA | 2365 |
| 127.861 | | | UCUGUUACCC | | | GUUAAGGGUU | |
| VoymiR- | D-2970 | 7776 | AACUCAUCUGU | 2366 | 7771 | UCAGGAUAACA | 2361 |
| 127.866 | | | UAUCUUGCCC | | | GAUGAGUUUU | |
| VoymiR- | D-2971 | 7777 | GCUGUGGAAAU | 2367 | 7778 | UAGGAUACAUU | 2368 |
| 127.870 | | | GUAUCUUCCC | | | UCUACAGCUU | |
| VoymiR- | D-2972 | 7792 | UGACUUGGGCA | 2382 | 7769 | UCCACCUUUGCC | 2359 |
| 127.823 | | | AAGGUAGCCC | | | CAAGUCAUU | |
| VOYmiR-120 | D-2973 | 7793 | CAAUGUGACUG | 2383 | 7794 | UUUGUCAGCAG | 2384 |
| | | | CUGACAAA | | | UCACAUUGUC | |
| dVOYmiR- | D-2974 | 7795 | GCAGGUCCUCA | 2385 | 7796 | GAUUAAAGUGA | 2386 |
| 102.788 | | | CUUUAAUGCC | | | GGACCUGCUU | |
| dVOYmiR- | D-2975 | 7797 | GGCAAUGUGAC | 2387 | 7798 | UGUCAGCAGUC | 2388 |
| 102.805 | | | UGCUGACCCC | | | ACAUUGCCUU | |
| dVOYmiR- | D-2976 | 7799 | GCAGGUCCUCA | 2389 | 7796 | GAUUAAAGUGA | 2386 |
| 104.788 | | | CUUUAAUUCC | | | GGACCUGCUU | |
| dVOYmiR- | D-2977 | 7800 | GGCAAUGUGAC | 2390 | 7798 | UGUCAGCAGUC | 2388 |
| 104.805 | | | UGCUGAUGCC | | | ACAUUGCCUU | |
| dVOYmiR- | D-2978 | 7801 | GCAGGUCCUCA | 2391 | 7796 | GAUUAAAGUGA | 2386 |
| 109.788 | | | CUUUAAUCCC | | | GGACCUGCUU | |
| dVOYmiR- | D-2979 | 7802 | GGCAAUGUGAC | 2392 | 7798 | UGUCAGCAGUC | 2388 |
| 109.805 | | | UGCUGAUACC | | | ACAUUGCCUU | |
| dVOYmiR- | D-2980 | 7803 | GCAGGUCCUGA | 2393 | 7796 | GAUUAAAGUGA | 2386 |
| 114.788 | | | CUUUAAUCCC | | | GGACCUGCUU | |
| dVOYmiR- | D-2981 | 7804 | GGCAAUGUGUC | 2394 | 7798 | UGUCAGCAGUC | 2388 |
| 114.805 | | | UGCUGAUACC | | | ACAUUGCCUU | |
| dVOYmiR- | D-2982 | 7801 | GCAGGUCCUCA | 2391 | 7796 | GAUUAAAGUGA | 2386 |
| 116.788 | | | CUUUAAUCCC | | | GGACCUGCUU | |
| dVOYmiR- | D-2983 | 7802 | GGCAAUGUGAC | 2392 | 7798 | UGUCAGCAGUC | 2388 |
| 116.805 | | | UGCUGAUACC | | | ACAUUGCCUU | |
| dVoymiR- | D-2984 | 7801 | GCAGGUCCUCA | 2391 | 7805 | GAUUAAAGUGA | 2395 |
| 127.788 | | | CUUUAAUCCC | | | GGACCUGCUUU | |
| dVoymiR- | D-2985 | 7802 | GGCAAUGUGAC | 2392 | 7806 | UGUCAGCAGUC | 2396 |
| 127.805 | | | UGCUGAUACC | | | ACAUUGCCUUU | |
| | D-4009 | S-4000 | GUCGUUUGGCU | 2522 | A- | UCACCACAAGCC | 2505 |
| | | | UGUGGUGGCU | | 4000 | AAACGACUU | |
| | D-4010 | S-4001 | CAGGUCCUCAC | 2523 | A- | UGAUUAAAGUG | 2506 |
| | | | UUUAAUCGCU | | 4001 | AGGACCUGUU | |
| | D-4011 | S-4002 | GCAGGUCCUCA | 2524 | A- | UAUUAAAGUGA | 2507 |
| | | | CUUUAAUGCC | | 4002 | GGACCUGCUU | |
| | D-4012 | S-4003 | GCAGGUCCUCA | 2525 | A- | UAUUAAAGUGA | 2507 |
| | | | CUUUAAUGCU | | 4002 | GGACCUGCUU | |
| | D-4013 | S-4004 | UCGUUUGGCUU | 2526 | A- | UACACCACAAG | 2508 |
| | | | GUGGUGUGCU | | 4003 | CCAAACGAUU | |
| | D-4015 | S-4006 | GUCGUUUGGCU | 2528 | A- | UCACCACAAGCC | 2510 |
| | | | UGUGGUGGCC | | 4005 | AAACGACUUU | |
| | D-4016 | S-4007 | CAGGUCCUCAC | 2529 | A- | UGAUUAAAGUG | 2506 |
| | | | UUUAAUCGCC | | 4001 | AGGACCUGUU | |
| | D-4017 | S-4008 | UCGUUUGGCUU | 2530 | A- | UACACCACAAG | 2508 |
| | | | GUGGUGUGCC | | 4003 | CCAAACGAUU | |
| | D-4018 | S-4009 | GGCAAUGUGAC | 2531 | A- | UGCCAGCAGUC | 2511 |
| | | | UGCUGGUGCC | | 4006 | ACAUUGCCUU | |
| | D-4019 | S-4010 | GGCAAUGUGAC | 2532 | A- | UGCCAGCAGUC | 2511 |
| | | | UGCUGGUACC | | 4006 | ACAUUGCCUU | |
| | D-4020 | S-4011 | GGCAAUGUGUC | 2533 | A- | UGCCAGCAGUC | 2511 |
| | | | UGCUGGUACC | | 4006 | ACAUUGCCUU | |
| | D-4021 | S-4012 | GGCAAUGUGAC | 2534 | A- | UGCCAGCAGUC | 2511 |
| | | | UGCUGGCCCC | | 4006 | ACAUUGCCUU | |

The passenger and guide strands are described in the Table 10. The “miR” component of the name of the sequence does not necessarily correspond to the sequence numbering of miRNA genes (e.g., VOYmiR-101 is the name of the sequence and does not necessarily mean that miR-101 is part of the sequence).

In some embodiments, the dTdT nucleotides of the sense strand and/or the antisense strand of the siRNA may be replaced with any two nucleotides (e.g., UU, UC, AA, AG, AC, etc.). Alternatively, the dTdT nucleotides of the sense strand and/or the antisense strand may be replaced with a single nucleotide (e.g., U, A, C, or G).

In other embodiments, the siRNA molecules of the present disclosure targeting SOD1 can be encoded in plasmid vectors, AAV particles, viral genome or other nucleic acid expression vectors for delivery to a cell.

DNA expression plasmids can be used to stably express the siRNA duplexes or dsRNA of the present disclosure targeting SOD1 in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

In other embodiments, the siRNA molecules of the present disclosure can be encoded in plasmid vectors, AAV particles, viral genome or other nucleic acid expression vectors for delivery to a cell.

In some embodiments, a siRNA for inhibiting expression of SOD1 is provided, wherein said siRNA comprises a sense strand and an antisense strand, the antisense strand comprising a region of complementarity to a SOD1 RNA transcript, which antisense strand comprises at least 15 (e.g., at least 16, 17, 18, 19, 20, 21, 22, or 23) contiguous nucleotides differing by no more than 3 (e.g., by no more than 0, 1 or 2) nucleotides from an antisense sequence listed in any one of the duplexes in Table 10. In one embodiment, the antisense sequence comprises at least 15 (e.g., at least 16, 17, 18, 19, 20, 21, 22, or 23) contiguous nucleotides differing by no more than 3 (e.g., by no more than 0, 1 or 2) nucleotides from the antisense sequence of SEQ ID NO: 2507, 2363 or 2365.

Additional sense and antisense strand sequences useful for the siRNA duplexes of the present application are disclosed in PCT Patent Application Publications WO2016077687, WO2019079240, WO2018204786, WO2019079242, WO 2020010042 and WO2020223296, which are hereby incorporated by reference in their entirety.

In some embodiments, the siRNA duplexes or encoded dsRNA of the present disclosure suppress (or degrade) the SOD1 mRNA. Accordingly, the siRNA duplexes or encoded dsRNA can be used to substantially inhibit SOD1 in a cell, for example a neuron.

The expression of SOD1 may be assessed based on the level of expression of SOD1 mRNA, SOD1 protein, or the level of another parameter functionally linked to the level of expression of SOD1. In some embodiments, the expression of SOD1 is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the siRNA has an IC₅₀ in the range of 0.001-0.01 nM, 0.001-0.10 nM, 0.001-1.0 nM, 0.001-10 nM, 0.01-0.05 nM, 0.01-0.50 nM, 0.02-0.60 nM, 0.01-1.0 nM, 0.01-1.5 nM, 0.01-10 nM. The IC₅₀ value may be normalized relative to an appropriate control value, e.g., the IC₅₀ of a non-targeting siRNA. Accordingly, the protein product of SOD1 may be inhibited. In some embodiments, the siRNA duplexes or encoded dsRNA of the present disclosure suppress (or degrade) the target mRNA in spinal cord motor neurons (e.g., RNA in vg+ cells of motor neuron morphology).

According to the present disclosure, the siRNA molecules are designed and tested for their ability in reducing SOD1 mRNA levels in cultured cells. Such siRNA molecules may form a duplex such as, but not limited to, include those listed in Table 10. As a non-limiting example, the siRNA duplexes may be siRNA duplex ID D-4012, D-2968, or D-2959.

In some embodiments, the siRNA duplex comprises: (i) a sense strand sequence comprising SEQ ID NO: 2525, and an antisense strand sequence comprising SEQ ID NO: 2507; (ii) a sense strand sequence comprising SEQ ID NO: 2376, and an antisense strand sequence comprising SEQ ID NO: 2365; or (iii) a sense strand sequence comprising SEQ ID NO: 2381, and an antisense strand sequence comprising SEQ ID NO: 2363.

In some embodiments, the sense strand sequence and the antisense strand sequence comprise a region of complementarity, wherein the region of complementarity is between 17-20 nucleotides in length.

In certain embodiments, the siRNA molecules comprise a miRNA seed match for SOD1 located in the guide strand. In another embodiment, the siRNA molecules comprise a miRNA seed match for SOD1 located in the passenger strand. In yet another embodiment, the siRNA duplexes or encoded dsRNA targeting SOD1 gene do not comprise a seed match for SOD1 located in the guide or passenger strand.

In certain embodiments, the siRNA duplex target SOD1 is designed so there is no miRNA seed match for the sense or antisense sequence to the non-SOD1 sequence.

In some embodiments, the siRNA duplexes or encoded dsRNA targeting SOD1 may have almost no significant full-length off target effects for the guide strand, and/or for the passenger strand.

In certain embodiments, the siRNA duplexes or encoded dsRNA targeting SOD1 may have high activity in vitro. In another embodiment, the siRNA molecules may have low activity in vitro. In yet another embodiment, the siRNA duplexes or dsRNA targeting SOD1 may have high guide strand activity and low passenger strand activity in vitro.

In certain embodiments, the siRNA molecules have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. As a non-limiting example, the target knock-down may persist for a period of time of at least 4 weeks, at least 8 weeks, at least 12 weeks, or more. In some embodiments, the highest knock-down from delivery of the siRNA molecules is seen around the injection site(s). In some embodiments, knock-down is seen in the ventral horn and around the injection site(s) after delivery of the siRNA molecules.

In certain embodiments, the 5′ processing of the guide strand of the siRNA duplex targeting SOD1 has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In certain embodiments, the 5′ processing of the guide strand of the siRNA duplex targeting SOD1 has a correct start (n) at the 5′ end in a range of 75-95%, 75-90%, 75-85%, 75-80%, 80-95%, 80-90%, 80-85%, 85-95%, 85-90%, or 90-95%. As a non-limiting example, the 5′ processing of the guide strand of the siRNA duplex targeting SOD1 has a correct start (n) at the 5′ end in a range of 75-95%.

In certain embodiments, the guide to passenger (G:P) (also referred to as the antisense to sense) strand ratio expressed is 1:10, 1:9, 1:8, 1:7, 1:6, 1:5, 1:4, 1:3, 1:2, 1:1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after intracellular processing of the pri-microRNA. For example, an 80:20 guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In certain embodiments, the guide to passenger (G:P) (also referred to as the antisense to sense) strand ratio expressed is greater than 1, greater than 2, greater than 5, greater than 10, greater than 20, greater than 50, or greater than 300.

In certain embodiments, the passenger to guide (P:G) (also referred to as the sense to antisense) strand ratio expressed is 1:10, 1:9, 1:8, 1:7, 1:6, 1:5, 1:4, 1:3, 1:2, 1:1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the intracellular processing of the pri-microRNA. For example, an 80:20 of passenger-to-guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In certain embodiments, the passenger to guide (P:G) (also referred to as the sense to antisense) strand ratio expressed is greater than 1, greater than 2, greater than 5, greater than 10, greater than 20, greater than 50, or greater than 300.

In certain embodiments, a passenger-guide strand duplex for SOD1 is considered effective when the pri- or pre-microRNAs demonstrate, buy methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In certain embodiments, the percent of guide strands to the total endogenous pool of miRNAs is 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, or 0.6%. As a non-limiting example, the percent is 0.06%. As a non-limiting example, the percent is 0.4%. As a non-limiting example, the percent is 0.5%. As a non-limiting example, the range is 0.06-0.6%. As a non-limiting example, the range is 0.4-0.5%.

In certain embodiments, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full-length sequence of the construct.

In certain embodiments, the siRNA molecules may be used to silence wild type or mutant version of the gene of interest by targeting at least one exon on the gene of interest sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67.

siRNA Modification

In some embodiments, the siRNA molecules of the present disclosure, when not delivered as a precursor or DNA, may be chemically modified to modulate some features of RNA molecules, such as, but not limited to, increasing the stability of siRNAs in vivo. The chemically modified siRNA molecules can be used in human therapeutic applications, and are improved without compromising the RNAi activity of the siRNA molecules. As a non-limiting example, the siRNA molecules modified at both the 3′ and the 5′ end of both the sense strand and the antisense strand.

In some aspects, the siRNA duplexes of the present disclosure may contain one or more modified nucleotides such as, but not limited to, sugar modified nucleotides, nucleobase modifications and/or backbone modifications. In some aspects, the siRNA molecule may contain combined modifications, for example, combined nucleobase and backbone modifications.

In some embodiments, the modified nucleotide may be a sugar-modified nucleotide. Sugar modified nucleotides include, but are not limited to 2′-fluoro, 2′-amino and 2′-thio modified ribonucleotides, e.g. 2′-fluoro modified ribonucleotides. Modified nucleotides may be modified on the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In some embodiments, the modified nucleotide may be a nucleobase-modified nucleotide.

In some embodiments, the modified nucleotide may be a backbone-modified nucleotide. In some embodiments, the siRNA duplexes of the present disclosure may further comprise other modifications on the backbone. A normal “backbone”, as used herein, refers to the repeating alternating sugar-phosphate sequences in a DNA or RNA molecule. The deoxyribose/ribose sugars are joined at both the 3′-hydroxyl and 5′-hydroxyl groups to phosphate groups in ester links, also known as “phosphodiester” bonds/linker (PO linkage). The PO backbones may be modified as “phosphorothioate backbone (PS linkage). In some cases, the natural phosphodiester bonds may be replaced by amide bonds but the four atoms between two sugar units are kept. Such amide modifications can facilitate the solid phase synthesis of oligonucleotides and increase the thermodynamic stability of a duplex formed with siRNA complement. See e.g. Mesmaeker et al., Pure & Appl. Chem., 1997, 3, 437-440; the content of which is incorporated herein by reference in its entirety.

Modified bases refer to nucleotide bases such as, for example, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

In certain embodiments, the modified nucleotides may be on just the sense strand, alternatively, the modified nucleotides may be on just the antisense strand, or the modified nucleotides may be in both the sense and antisense strands.

In some embodiments, the chemically modified nucleotide does not affect the ability of the antisense strand to pair with the target mRNA sequence.

In certain embodiments, the AAV particle comprising a nucleic acid sequence encoding the siRNA molecules of the present disclosure may encode siRNA molecules which are polycistronic molecules. The siRNA molecules may additionally comprise one or more linkers between regions of the siRNA molecules.

### Molecular Scaffold

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the molecular scaffold comprises at least one 5′ flanking region. As a non-limiting example, the 5′ flanking region may comprise a 5′ flanking sequence which may be of any length and may be derived in whole or in part from wild type microRNA sequence or be a completely artificial sequence.

In some embodiments, one or both of the 5′ and 3′ flanking sequences are absent.

In some embodiments the 5′ and 3′ flanking sequences are the same length.

In some embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 5′ flanking sequence may be about 1, about 5, about 10, about 15, about 20, about 30, about 35, about 40, about 50, about 60, about 80, about 100, about 150, about 200, about 250, about 300, about 350, about 400, or about 500 nucleotides in length. In some embodiments, the 5′ flanking sequence is 30 nucleotides in length, 54 nucleotides in length, or 100 nucleotides in length.

In some embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 3′ flanking sequence may be about 1, about 5, about 10, about 15, about 20, about 30, about 35, about 40, about 50, about 60, about 80, about 100, about 150, about 200, about 250, about 300, about 350, about 400, or about 500 nucleotides in length. In some embodiments, the 3′ flanking sequence is 35 nucleotides in length, 52 nucleotides in length, 99 nucleotides in length, or 100 nucleotides in length.

In some embodiments the 5′ and 3′ flanking sequences are the same sequence. In some embodiments they differ by 2%, 3%, 4%, 5%, 10%, 20% or more than 30% when aligned to each other. In some embodiments the 5′ and 3′ flanking regions may differ by no more than 5, 4, 3, 2 or 1 nucleotides from each other.

In certain embodiments, the molecular scaffold comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In certain embodiments, the molecular scaffold comprises at least one loop motif region. As a non-limiting example, the loop motif region may comprise a sequence which may be of any length.

In certain embodiments, the molecular scaffold comprises a 5′ flanking region, a loop motif region and/or a 3′ flanking region.

In certain embodiments, at least one siRNA, miRNA or other RNAi agent described herein, may be encoded by a modulatory polynucleotide which may also comprise at least one molecular scaffold. The molecular scaffold may comprise a 5′ flanking sequence which may be of any length and may be derived in whole or in part from wild type microRNA sequence or be completely artificial. The 3′ flanking sequence may mirror the 5′ flanking sequence and/or a 3′ flanking sequence in size and origin. Either flanking sequence may be absent. The 3′ flanking sequence may optionally contain one or more CNNC motifs, where “N” represents any nucleotide.

Forming the stem of a stem loop structure is a minimum of the modulatory polynucleotide encoding at least one siRNA, miRNA or other RNAi agent described herein. In some embodiments, the siRNA, miRNA or other RNAi agent described herein comprises at least one nucleic acid sequence which is in part complementary or will hybridize to a target sequence. In some embodiments the payload is an siRNA molecule or fragment of an siRNA molecule.

In some embodiments, the 5′ arm of the stem loop structure of the modulatory polynucleotide comprises a nucleic acid sequence encoding a sense sequence. Non-limiting examples of sense sequences, or fragments or variants thereof, which may be encoded by the modulatory polynucleotide are described in Table 10. In some embodiments, the nucleic acid sequence encodes a sense sequence which comprises at least 15 (e.g., at least 16, 17, 18, 19, 20, 21, 22, or 23) contiguous nucleotides differing by no more than 3, 2 or 1 nucleotides from any one of the sense sequences in Table 10.

In some embodiments, the 3′ arm of the stem loop of the modulatory polynucleotide comprises a nucleic acid sequence encoding an antisense sequence. The antisense sequence, in some instances, comprises a “G” nucleotide at the 5′ most end. Non-limiting examples of antisense sequences, or fragments or variants thereof, which may be encoded by the modulatory polynucleotide are described in Table 10. In some embodiments, the nucleic acid sequence encodes an antisense sequence which comprises at least 15 (e.g., at least 16, 17, 18, 19, 20, 21, 22, or 23) contiguous nucleotides differing by no more than 3, 2 or 1 nucleotides from any one of the antisense sequences in Table 10.

In other embodiments, the sense sequence may reside on the 3′ arm while the antisense sequence resides on the 5′ arm of the stem of the stem loop structure of the modulatory polynucleotide. Non-limiting examples of sense and antisense sequences which may be encoded by the modulatory polynucleotide are described in Table 10.

In certain embodiments, the sense and antisense sequences may be completely complementary across a substantial portion of their length. In other embodiments the sense sequence and antisense sequence may be at least 70, 80, 90, 95 or 99% complementarity across independently at least 50, 60, 70, 80, 85, 90, 95, or 99% of the length of the strands. The sense strand sequence and an antisense strand sequence may be 100% complementary to each other, or alternatively, they may have 1, 2, 3, 4, or more mismatches. These mismatches may be contiguous, or alternatively, they may be non-contiguous throughout the sequence. In some embodiments, the mismatched nucleotides are located at the ends of the sequences. In some embodiments, the mismatched nucleotides of the sense and antisense sequence are on the 5′ end of the sequences. Alternatively, the mismatch may be on the 3′ end of the sense and antisense sequences.

Neither the identity of the sense sequence nor the homology of the antisense sequence need to be 100% complementarity to the target sequence.

In certain embodiments, separating the sense and antisense sequence of the stem loop structure of the modulatory polynucleotide is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9 nucleotides, 10 nucleotides, 11 nucleotides, 12 nucleotides, 13 nucleotides, 14 nucleotides, and/or 15 nucleotides.

In some embodiments, the loop sequence comprises a nucleic acid sequence encoding at least one UGUG motif. In some embodiments, the nucleic acid sequence encoding the UGUG motif is located at the 5′ terminus of the loop sequence.

In certain embodiments, spacer regions may be present in the modulatory polynucleotide to separate one or more modules (e.g., 5′ flanking region, loop motif region, 3′ flanking region, sense sequence, antisense sequence) from one another. There may be one or more such spacer regions present.

In certain embodiments, a spacer region of between 8-20, i.e., 8, 9, 10, 11, 12, 13, 14, 15, 16, 17, 18, 19, or 20 nucleotides may be present between the sense sequence and a flanking region sequence.

In certain embodiments, the length of the spacer region is 13 nucleotides and is located between the 5′ terminus of the sense sequence and the 3′ terminus of the flanking sequence. In certain embodiments, a spacer is of sufficient length to form approximately one helical turn of the sequence.

In certain embodiments, a spacer region of between 8-20, i.e., 8, 9, 10, 11, 12, 13, 14, 15, 16, 17, 18, 19, or 20 nucleotides may be present between the antisense sequence and a flanking sequence.

In certain embodiments, the spacer sequence is between 10-13, i.e., 10, 11, 12 or 13 nucleotides and is located between the 3′ terminus of the antisense sequence and the 5′ terminus of a flanking sequence. In certain embodiments, a spacer is of sufficient length to form approximately one helical turn of the sequence.

In certain embodiments, the molecular scaffold of the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a nucleic acid sequence encoding a sense sequence and the 3′ arm comprises a nucleic acid sequence encoding the antisense sequence. In another non-limiting example, the 5′ arm comprises a nucleic acid sequence encoding the antisense sequence and the 3′ arm comprises a nucleic acid sequence encoding the sense sequence.

In certain embodiments, the 5′ arm, sense and/or antisense sequence, loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In certain embodiments, the molecular scaffold of the modulatory polynucleotides is aligned in order to have the rate of excision of the guide strand (also referred to herein as the antisense strand) be greater than the rate of excision of the passenger strand (also referred to herein as the sense strand). The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In certain embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In certain embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In certain embodiments, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In certain embodiments, the molecular scaffold comprises a dual-function targeting modulatory polynucleotide. As used herein, a “dual-function targeting” modulatory polynucleotide is a polynucleotide where both the guide and passenger strands knock down the same target or the guide and passenger strands knock down different targets.

In certain embodiments, the molecular scaffold of the modulatory polynucleotides described herein may comprise a 5′ flanking region, a loop motif region and a 3′ flanking region. Non-limiting examples of the sequences for the 5′ flanking region, loop motif region (may also be referred to as a linker region) and the 3′ flanking region which may be used, or fragments thereof used, in the modulatory polynucleotides described herein are shown in Tables 11-13.

TABLE 11
5′ Flanking Regions for Molecular Scaffold

| 5′ | | 5′ |
|---|---|---|
| Flanking | | Flanking |
| Region | | Region |
| Name | 5′ Flanking Region Sequence | SEQ ID |
| 5F1 | CTCCCGCAGAACACCATGCGCTCCACGGAA | 2547 |
| 5F2 | GAAGCAAAGAAGGGGCAGAGGGAGCCCGTG | 2548 |
| | AGCTGAGTGGGCCAGGGACTGGGAGAAGGA | |
| | GTGAGGAGGCAGGGCCGGCATGCCTCTGCT | |
| | GCTGGCCAGA | |
| 5F3 | GTGCTGGGCGGGGGGCGGCGGGCCCTCCCG | 2549 |
| | CAGAACACCATGCGCTCTTCGGAA | |
| 5F4 | GTGCTGGGCGGGGGGCGGCGGGCCCTCCCG | 5014 |
| | CAGAACACCATGCGCTCCACGGAA | |

TABLE 12
Loop Motif Regions for Molecular Scaffold

| Loop Motif | Loop Motif Region | Loop Motif |
|---|---|---|
| Region Name | Sequence | Region SEQ ID |
| L1 | GTGGCCACTGAGAAG | 2550 |
| L2 | GTCTGCACCTGTCACTAG | 2551 |
| L3 | TGTGACCTGG | 2552 |
| L4 | TGTGATTTGG | 2553 |

TABLE 13
3′ Flanking Regions for Molecular Scaffold

| 3′ | | 3′ |
|---|---|---|
| Flanking | | Flanking |
| Region | | Region |
| Name | 3′ Flanking Region Sequence | SEQ ID |
| 3F1 | CTGAGGAGCGCCTTGACAGCAGCCATGGGAG | 2554 |
| | GGCC | |
| 3F2 | TGGCCGTGTAGTGCTACCCAGCGCTGGCTGCC | 2555 |
| | TCCTCAGCATTGCAATTCCTCTCCCATCTGGG | |
| | CACCAGTCAGCTACCCTGGTGGGAATCTGGGT | |
| | AGCC | |
| 3F3 | GGCCGTGTAGTGCTACCCAGCGCTGGCTGCCT | 2556 |
| | CCTCAGCATTGCAATTCCTCTCCCATCTGGGC | |
| | ACCAGTCAGCTACCCTGGTGGGAATCTGGGT | |
| | AGCC | |
| 3F4 | CTGAGGAGCGCCTTGACAGCAGCCATGGGAG | 2557 |
| | GGCCGCCCCCTACCTCAGTGA | |
| 3F5 | CTGTGGAGCGCCTTGACAGCAGCCATGGGAG | 2558 |
| | GGCCGCCCCCTACCTCAGTGA | |

In certain embodiments, the molecular scaffold may comprise at least one 5′ flanking region, fragment or variant thereof listed in Table 11. In some embodiments, the molecular scaffold may comprise a 5′ Flanking Region sequence which comprises at least 15 (e.g., at least 16, 17, 18, 19, 20, 21, 22, or 23) contiguous nucleotides differing by no more than 3, 2 or 1 nucleotides from any one of the 5′ Flanking Region sequences in Table 11. These differing nucleotides may be contiguous, or alternatively, they may be non-contiguous throughout the sequence. In some embodiments, the differing nucleotides are located at the ends of the sequences. As a non-limiting example, the 5′ flanking region may be 51F4. In some embodiments, the AAV particle includes nucleotide sequence of SEQ ID NO: 2547, 2548, 2549, or 5014, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2547, 2548, 2549, or 5014.

In certain embodiments, the molecular scaffold may comprise at least one 5F1 flanking region.

In certain embodiments, the molecular scaffold may comprise at least one loop motif region, fragment or variant thereof listed in Table 12. In some embodiments, the molecular scaffold may comprise a 3′ Flanking Region sequence which comprises at least 8 (e.g., at least 9, 10, 11, 12, 13, 14, or 15) contiguous nucleotides differing by no more than 3, 2 or 1 nucleotides from any one of the a 3′ Flanking Region sequences in Table 12. These differing nucleotides may be contiguous, or alternatively, they may be non-contiguous throughout the sequence. In some embodiments, the differing nucleotides are located at the ends of the sequences. In some embodiments, the AAV particle includes nucleotide sequence of SEQ ID NO: 2550, 2551, 2552, or 2553, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, or four, modifications, but no more than six modifications of SEQ ID NO: 2550, 2551, 2552, or 2553. As a non-limiting example, the loop motif region may be L1.

In certain embodiments, the molecular scaffold may comprise at least one L1 loop motif region.

In certain embodiments, the molecular scaffold may comprise at least one 3′ flanking region, fragment or variant thereof listed in Table 13. In some embodiments, the molecular scaffold may comprise a loop region sequence which comprises at least 15 (e.g., at least 16, 17, 18, 19, 20, 21, 22, or 23) contiguous nucleotides differing by no more than 3, 2 or 1 nucleotides from any one of the loop sequences in Table 12. These differing nucleotides may be contiguous, or alternatively, they may be non-contiguous throughout the sequence. In some embodiments, the differing nucleotides are located at the ends of the sequences. In some embodiments, the AAV particle includes nucleotide sequence of SEQ ID NO: 2554, 2555, 2556, 2557, or 2558, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2554, 2555, 2556, 2557, or 2558. As a non-limiting example, the 3′ flanking region may be 3F1.

In certain embodiments, the molecular scaffold may comprise at least one 3F1 flanking region.

In certain embodiments, the molecular scaffold may comprise at least one 5′ flanking region, fragment or variant thereof, and at least one loop motif region, fragment or variant thereof, as described in Tables 11 and 12. As a non-limiting example, the 5′ flanking region and the loop motif region may be 5F1 and L1.

In certain embodiments, the molecular scaffold may comprise at least one 3′ flanking region, fragment or variant thereof, and at least one motif region, fragment or variant thereof, as described in Tables 12 and 13. As a non-limiting example, the 3′ flanking region and the loop motif region may be 3F1 and L1.

In certain embodiments, the molecular scaffold may comprise at least one 5′ flanking region, fragment or variant thereof, and at least one 3′ flanking region, fragment or variant thereof, as described in Tables 11 and 13. As a non-limiting example, the flanking regions may be 5F1 and 3F1.

In certain embodiments, the molecular scaffold may comprise at least one 5′ flanking region, fragment or variant thereof, at least one loop motif region, fragment or variant thereof, and at least one 3′ flanking region as described in Tables 11-13. As a non-limiting example, the flanking and loop motif regions may be 5F1, L1 and 3F1.

In certain embodiments, the molecular scaffold may be a natural pri-miRNA scaffold. As a non-limiting example, the molecular scaffold may be a scaffold derived from the human miR155 scaffold.

In certain embodiments, the molecular scaffold may comprise one or more linkers known in the art. The linkers may separate regions or one molecular scaffold from another. As a non-limiting example, the molecular scaffold may be polycistronic.

Additional 5′ flanking region nucleotides, loop region nucleotides, and 3′ flanking region nucleotides that may be used in the molecular scaffold of the modulatory polynucleotides described herein are disclosed in PCT Patent Application Publications WO2016077687, WO2019079240, WO2018204786, WO2019079242, WO 2020010042 and WO202022329625, which are hereby incorporated by reference in their entirety.

Modulatory Polynucleotide Comprising Molecular Scaffold and siRNA Molecules Targeting SOD1

In certain embodiments, the modulatory polynucleotide may comprise 5′ and 3′ flanking regions, loop motif region, and nucleic acid sequences encoding sense sequence and antisense sequence as described in Table 14. In Table 14, the DNA sequence identifier for the passenger and guide strands are described as well as the 5′ and 3′ Flanking Regions and the Loop region (also referred to as the linker region). In Table 14, the “miR” component of the name of the sequence does not necessarily correspond to the sequence numbering of miRNA genes (e.g., VOYSOD1miR-102 is the name of the sequence and does not necessarily mean that miR-102 is part of the sequence).

TABLE 14
SOD1 Modulatory Polynucleotide Sequence Regions (5′ to 3′)

| Modulatory | SEQ | | |
|---|---|---|---|
| Polynucleotide | ID | | |
| Construct Name | NO | Description | Sequence |
| VOYSOD1miR104- | 2562 | 5′ Flanking to | CTCCCGCAGAACACCATGCGCTCCACGGAAGCAGGTCCTCA |
| 788.2 | | 3′ Flanking | CTTTAATGCTGTGGCCACTGAGAAGTATTAAAGTGAGGACC |
| | | | TGCTTCTGAGGAGCGCCTTGACAGCAGCCATGGGAGGGCC |
| | 2547 | 5′ Flanking | CTCCCGCAGAACACCATGCGCTCCACGGAA |
| | 2583 | Passenger | GCAGGTCCTCACTTTAATGCT |
| | 2550 | Loop | GTGGCCACTGAGAAG |
| | 2604 | Guide | TATTAAAGTGAGGACCTGCTT |
| | 2554 | 3′Flanking | CTGAGGAGCGCCTTGACAGCAGCCATGGGAGGGCC |
| VOYSOD1miR127- | 2579 | 5′ Flanking to | GAAGCAAAGAAGGGGCAGAGGGAGCCCGTGAGCTGAGTGGG |
| 860 | | 3′ Flanking | CCAGGGACTGGGAGAAGGAGTGAGGAGGCAGGGCCGGCATG |
| | | | CCTCTGCTGCTGGCCAGACCCCTTAACTCATTTGTTCCCGT |
| | | | CTGCACCTGTCACTAGTAACAGATGAGTTAAGGGGTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2600 | Passenger | CCCCTTAACTCATTTGTTCCC |
| | 2551 | Loop | GTCTGCACCTGTCACTAG |
| | 2621 | Guide | TAACAGATGAGTTAAGGGGTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR114- | 5022 | 5′ Flanking to | GTGCTGGGCGGGGGGCGGCGGGCCCTCCCGCAGAACACCAT |
| 861 | | 3′ Flanking | GCGCTCTTCGGAACCCTTAACTGATCTGTTAACCTGTGACC |
| | | | TGGTTAACAGATGAGTTAAGGGTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 5023 | Passenger | CCCTTAACTGATCTGTTAACC |
| | 2552 | Loop | TGTGACCTGG |
| | 5024 | Guide | TTAACAGATGAGTTAAGGGTT |
| | 2558 | 3′Flanking | CTGTGGAGCGCCTTGACAGCAGCCATGGGAGGGCCGCCCCC |
| | | | TACCTCAGTGA |
| VOYSOD1miR102- | 2559 | 5′ Flanking to | GTGCTGGGCGGGGGGCGGCGGGCCCTCCCGCAGAACACCAT |
| 788 | | 3′ Flanking | GCGCTCTTCGGAAGCAGGTCCTCACTTTAATGCCTGTGACC |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2580 | Passenger | GCAGGTCCTCACTTTAATGCC |
| | 2552 | Loop | TGTGACCTGG |
| | 2601 | Guide | GATTAAAGTGAGGACCTGCTT |
| | 2557 | 3′Flanking | CTGAGGAGCGCCTTGACAGCAGCCATGGGAGGGCCGCCCCC |
| | | | TACCTCAGTGA |
| VOYSOD1miR102- | 2560 | 5′ Flanking to | GTGCTGGGCGGGGGGCGGCGGGCCCTCCCGCAGAACACCAT |
| 805c | | 3′ Flanking | GCGCTCTTCGGAAGGCAATGTGACTGCTGGCCCCTGTGACC |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2581 | Passenger | GGCAATGTGACTGCTGGCCCC |
| | 2552 | Loop | TGTGACCTGG |
| | 2602 | Guide | TGCCAGCAGTCACATTGCCTT |
| | 2557 | 3′Flanking | CTGAGGAGCGCCTTGACAGCAGCCATGGGAGGGCCGCCCCC |
| | | | TACCTCAGTGA |
| VOYSOD1miR104- | 2561 | 5′ Flanking to | GTGCTGGGCGGGGGGCGGCGGGCCCTCCCGCAGAACACCAT |
| 788 | | 3′ Flanking | GCGCTCTTCGGAAGCAGGTCCTCACTTTAATTCCTGTGACC |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGG TCCCGCAGAACACCAT |
| | | | GCGCTCTTCGGAA |
| | 2582 | Passenger | GCAGGTCCTCACTTTAATTCC |
| | 2552 | Loop | TGTGACCTGG |
| | 2603 | Guide | GATTAAAGTGAGGACCTGCTT |
| | 2557 | 3′Flanking | CTGAGGAGCGCCTTGACAGCAGCCATGGGAGGGCCGCCCCC |
| | | | TACCTCAGTGA |
| VOYSOD1miR104- | 2563 | 5′ Flanking to | CTCCCGCAGAACACCATGCGCTCCACGGAACAGGTCCTCAC |
| 789 | | 3′ Flanking | TTTAATCGCTGTGGCCACTGAGAAGTGATTAAAGTGAGGAC |
| | | | CTGTTCTGAGGAGCGCCTTGACAGCAGCCATGGGAGGGCC |
| | 2547 | 5′ Flanking | CTCCCGCAGAACACCATGCGCTCCACGGAA |
| | 2584 | Passenger | CAGGTCCTCACTTTAATCGCT |
| | 2550 | Loop | GTGGCCACTGAGAAG |
| | 2605 | Guide | TGATTAAAGTGAGGACCTGTT |
| | 2554 | 3′Flanking | CTGAGGAGCGCCTTGACAGCAGCCATGGGAGGGCC |
| VOYSOD1miR104- | 2564 | 5′ Flanking to | GTGCTGGGCGGGGGGCGGCGGGCCCTCCCGCAGAACACCAT |
| 805c | | 3′ Flanking | GCGCTCTTCGGAAGGCAATGTGACTGCTGGTGCCTGTGACC |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGC( CTCCCGCAGAACACCAT |
| | | | GCGCTCTTCGGAA |
| | 2585 | Passenger | GGCAATGTGACTGCTGGTGCC |
| | 2552 | Loop | TGTGACCTGG |
| | 2606 | Guide | TGCCAGCAGTCACATTGCCTT |
| | 2557 | 3′Flanking | CTGAGGAGCGCCTTGACAGCAGCCATGGGAGGGCCGCCCCC |
| | | | TACCTCAGTGA |
| VOYSOD1miR104- | 2565 | 5′ Flanking to | CTCCCGCAGAACACCATGCGCTCCACGGAAGTCGTTTGGCT |
| 829 | | 3′ Flanking | TGTGGTGGCTGTGGCCACTGAGAAGTCACCACAAGCCAAAC |
| | | | GACTTCTGAGGAGCGCCTTGACAGCAGCCATGGGAGGGCC |
| | 2547 | 5′ Flanking | CTCCCGCAGAACACCATGCGCTCCACGGAA |
| | 2586 | Passenger | GTCGTTTGGCTTGTGGTGGCT |
| | 2550 | Loop | GTGGCCACTGAGAAG |
| | 2607 | Guide | TCACCACAAGCCAAACGACTT |
| | 2554 | 3′Flanking | CTGAGGAGCGCCTTGACAGCAGCCATGGGAGGGCC |
| VOYSOD1miR104- | 2566 | 5′ Flanking to | CTCCCGCAGAACACCATGCGCTCCACGGAATCGTTTGGCTT |
| 830 | | 3′ Flanking | GTGGTGTGCTGTGGCCACTGAGAAGTACACCACAAGCCAAA |
| | | | CGATTCTGAGGAGCGCCTTGACAGCAGCCATGGGAGGGCC |
| | 2547 | 5′ Flanking | CTCCCGCAGAACACCATGCGCTCCACGGAA |
| | 2587 | Passenger | TCGTTTGGCTTGTGGTGTGCT |
| | 2550 | Loop | GTGGCCACTGAGAAG |
| | 2608 | Guide | TACACCACAAGCCAAACGATT |
| | 2554 | 3′Flanking | CTGAGGAGCGCCTTGACAGCAGCCATGGGAGGGCC |
| VOYSOD1miR109- | 2567 | 5′ Flanking to | GTGCTGGGCGGGGGGCGGCGGGCCCTCCCGCAGAACACCAT |
| 788 | | 3′Flanking | GCGCTCTTCGGAAGCAGGTCCTCACTTTAATCCCTGTGATT |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2588 | Passenger | GCAGGTCCTCACTTTAATCCC |
| | 2553 | Loop | TGTGATTTGG |
| | 2609 | Guide | GATTAAAGTGAGGACCTGCTT |
| | 2557 | 3′ Flanking | CTGAGGAGCGCCTTGACAGCAGCCATGGGAGGGCCGCCCCC |
| | | | TACCTCAGTGA |
| VOYSOD1miR109- | 2568 | 5′ Flanking to | GTGCTGGGCGGGGGGCGGCGGGCCCTCCCGCAGAACACCAT |
| 805c | | 3′ Flanking | GCGCTCTTCGGAAGGCAATGTGACTGCTGGTACCTGTGATT |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2589 | Passenger | GGCAATGTGACTGCTGGTACC |
| | 2553 | Loop | TGTGATTTGG |
| | 2610 | Guide | TGCCAGCAGTCACATTGCCTT |
| | 2557 | 3′Flanking | CTGAGGAGCGCCTTGACAGCAGCCATGGGAGGGCCGCCCCC |
| | | | TACCTCAGTGA |
| VOYSOD1miR114- | 2569 | 5′ Flanking to | GTGCTGGGCGGGGGGCGGCGGGCCCTCCCGCAGAACACCAT |
| 788 | | 3′ Flanking | GCGCTCTTCGGAAGCAGGTCCTGACTTTAATCCCTGTGACC |
| | | | TGGGATTAAAGTGAGGACCTGCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2590 | Passenger | GCAGGTCCTGACTTTAATCCC |
| | 2552 | Loop | TGTGACCTGG |
| | 2611 | Guide | GATTAAAGTGAGGACCTGCTT |
| | 2558 | 3′Flanking | CTGTGGAGCGCCTTGACAGCAGCCATGGGAGGGCCGCCCCC |
| | | | TACCTCAGTGA |
| VOYSOD1miR114- | 2570 | 5′ Flanking to | GTGCTGGGCGGGGGGCGGCGGGCCCTCCCGCAGAACACCAT |
| 805c | | 3′ Flanking | GCGCTCTTCGGAAGGCAATGTGTCTGCTGGTACCTGTGACC |
| | | | TGGTGCCAGCAGTCACATTGCCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2591 | Passenger | GGCAATGTGTCTGCTGGTACC |
| | 2552 | Loop | TGTGACCTGG |
| | 2612 | Guide | TGCCAGCAGTCACATTGCCTT |
| | 2558 | 3′Flanking | CTGTGGAGCGCCTTGACAGCAGCCATGGGAGGGCCGCCCCC |
| | | | TACCTCAGTGA |
| VOYSOD1miR116- | 2571 | 5′ Flanking to | GTGCTGGGCGGGGGGCGGCGGGCCCTCCCGCAGAACACCAT |
| 788 | | 3′ Flanking | GCGCTCTTCGGAAGCAGGTCCTCACTTTAATCCCTGTGACC |
| | | | TGGGATTAAAGTGAGGACCTGCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2592 | Passenger | GCAGGTCCTCACTTTAATCCC |
| | 2552 | Loop | TGTGACCTGG |
| | 2613 | Guide | GATTAAAGTGAGGACCTGCTT |
| | 2558 | 3′Flanking | CTGTGGAGCGCCTTGACAGCAGCCATGGGAGGGCCGCCCCC |
| | | | TACCTCAGTGA |
| VOYSOD1miR116- | 2572 | 5′ Flanking to | GTGCTGGGCGGGGGGCGGCGGGCCCTCCCGCAGAACACCAT |
| 805c | | 3′ Flanking | GCGCTCTTCGGAAGGCAATGTGACTGCTGGTACCTGTGACC |
| | | | TGGTGCCAGCAGTCACATTGCCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 2593 | Passenger | GGCAATGTGACTGCTGGTACC |
| | 2552 | Loop | TGTGACCTGG |
| | 2614 | Guide | TGCCAGCAGTCACATTGCCTT |
| | 2558 | 3′Flanking | CTGTGGAGCGCCTTGACAGCAGCCATGGGAGGGCCGCCCCC |
| | | | TACCTCAGTGA |
| VOYSOD1miR127- | 2573 | 5′ Flanking to | GAAGCAAAGAAGGGGCAGAGGGAGCCCGTGAGCTGAGTGGG |
| 788 | | 3′ Flanking | CCAGGGACTGGGAGAAGGAGTGAGGAGGCAGGGCCGGCATG |
| | | | CCTCTGCTGCTGGCCAGAGCAGGTCCTCACTTTAATCCCGT |
| | | | CTGCACCTGTCACTAGGATTAAAGTGAGGACCTGCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2594 | Passenger | GCAGGTCCTCACTTTAATCCC |
| | 2551 | Loop | GTCTGCACCTGTCACTAG |
| | 2615 | Guide | GATTAAAGTGAGGACCTGCTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR127- | 2574 | 5′ Flanking to | GAAGCAAAGAAGGGGCAGAGGGAGCCCGTGAGCTGAGTGGG |
| 788.2 | | 3′ Flanking | CCAGGGACTGGGAGAAGGAGTGAGGAGGCAGGGCCGGCATG |
| | | | CCTCTGCTGCTGGCCAGAGCAGGTCCTCACTTTAATGCCGT |
| | | | CTGCACCTGTCACTAGTATTAAAGTGAGGACCTGCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2595 | Passenger | GCAGGTCCTCACTTTAATGCC |
| | 2551 | Loop | GTCTGCACCTGTCACTAG |
| | 2616 | Guide | TATTAAAGTGAGGACCTGCTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR127- | 2575 | 5′ Flanking to | GAAGCAAAGAAGGGGCAGAGGGAGCCCGTGAGCTGAGTGGG |
| 789 | | 3′ Flanking | CCAGGGACTGGGAGAAGGAGTGAGGAGGCAGGGCCGGCATG |
| | | | CCTCTGCTGCTGGCCAGACAGGTCCTCACTTTAATCGCCGT |
| | | | CTGCACCTGTCACTAGTGATTAAAGTGAGGACCTGTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2596 | Passenger | CAGGTCCTCACTTTAATCGCC |
| | 2551 | Loop | GTCTGCACCIGTCACTAG |
| | 2617 | Guide | TGATTAAAGTGAGGACCTGTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR127- | 2576 | 5′ Flanking to | GAAGCAAAGAAGGGGCAGAGGGAGCCCGTGAGCTGAGTGGG |
| 805c | | 3′ Flanking | CCAGGGACTGGGAGAAGGAGTGAGGAGGCAGGGCCGGCATG |
| | | | CCTCTGCTGCTGGCCAGAGGCAATGTGACTGCTGGTACCGT |
| | | | CTGCACCTGTCACTAGTGCCAGCAGTCACATTGCCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2597 | Passenger | GGCAATGTGACTGCTGGTACC |
| | 2551 | Loop | GTCTGCACCTGTCACTAG |
| | 2618 | Guide | TGCCAGCAGTCACATTGCCTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR127- | 2577 | 5′ Flanking to | GAAGCAAAGAAGGGGCAGAGGGAGCCCGTGAGCTGAGTGGG |
| 829 | | 3′ Flanking | CCAGGGACTGGGAGAAGGAGTGAGGAGGCAGGGCCGGCATG |
| | | | CCTCTGCTGCTGGCCAGAGTCGTTTGGCTTGTGGTGGCCGT |
| | | | CTGCACCTGTCACTAGTCACCACAAGCCAAACGACTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2598 | Passenger | GTCGTTTGGCTTGTGGTGGCC |
| | 2551 | Loop | GTCTGCACCTGTCACTAG |
| | 2619 | Guide | TCACCACAAGCCAAACGACTTT |
| | 2556 | 3′Flanking | GGCCGTGTAGTGCTACCCAGCGCTGGCTGCCTCCTCAGCAT |
| | | | TGCAATTCCTCTCCCATCTGGGCACCAGTCAGCTACCCTGG |
| | | | TGGGAATCTGGGTAGCC |
| VOYSOD1miR127- | 2578 | 5′ Flanking to | GAAGCAAAGAAGGGGCAGAGGGAGCCCGTGAGCTGAGTGGG |
| 830 | | 3′ Flanking | CCAGGGACTGGGAGAAGGAGTGAGGAGGCAGGGCCGGCATG |
| | | | CCTCTGCTGCTGGCCAGATCGTTTGGCTTGTGGTGTGCCGT |
| | | | CTGCACCTGTCACTAGTACACCACAAGCCAAACGATTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 2599 | Passenger | TCGTTTGGCTTGTGGTGTGCC |
| | 2551 | Loop | GTCTGCACCTGTCACTAG |
| | 2620 | Guide | TACACCACAAGCCAAACGATT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |

In some embodiments, the molecular scaffold may comprise a 5′ Flanking to 3′ Flanking Region sequence which comprises at least 20 (e.g., at least 21, 22, 23, 24, 25, 26, 27, 28, 29, or 30) contiguous nucleotides differing by no more than 5, 4, 3, 2 or 1 nucleotides from any one of the 5′ Flanking to 3′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the nucleotide sequence encoding the siRNA duplex comprises the nucleotide sequence of any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 70% (e.g., at least 75%, 80%, 85%, 90%, 92%, 95%, 97%, 98%, or 99%) identical thereto.

In some embodiments, the molecular scaffold may comprise a 5′ Flanking Region sequence which comprises at least 15 (e.g., at least 16, 17, 18, 19, 20, 21, 22, or 23) contiguous nucleotides differing by no more than 3, 2 or 1 nucleotides from any one of the 5′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the 5′ Flanking Region includes a nucleotide from any one of the 5′ Flanking Region sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a passenger sequence which comprises at least 15 (e.g., at least 16, 17, 18, 19, 20, 21, 22, or 23) contiguous nucleotides differing by no more than 3, 2 or 1 nucleotides from any one of the passenger sequences in Table 14. These differing nucleotides may be contiguous, or alternatively, they may be non-contiguous throughout the sequence. In some embodiments, the differing nucleotides are located at the ends of the sequences. In some embodiments, the passenger sequence includes a nucleotide from any one of the passenger sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a loop sequence which comprises at least 7 (e.g., at least 8, 9, or 10) contiguous nucleotides differing by no more than 3, 2 or 1 nucleotides from any one of the Loop sequences in Table 14. These differing nucleotides may be contiguous, or alternatively, they may be non-contiguous throughout the sequence. In some embodiments, the differing nucleotides are located at the ends of the sequences. In some embodiments, the loop sequence includes a nucleotide from any one of the loop sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a guide sequence which comprises at least 15 (e.g., at least 16, 17, 18, 19, 20, 21, 22, or 23) contiguous nucleotides differing by no more than 3, 2 or 1 nucleotides from any one of the guide sequences in Table 14. These differing nucleotides may be contiguous, or alternatively, they may be non-contiguous throughout the sequence. In some embodiments, the differing nucleotides are located at the ends of the sequences. In some embodiments, the guide sequence includes a nucleotide from any one of the guide sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a 3′ Flanking Region sequence which comprises at least 15 (e.g., at least 16, 17, 18, 19, 20, 21, 22, or 23) contiguous nucleotides differing by no more than 3, 2 or 1 nucleotides from any one of the 3′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the 3′ Flanking Region sequence includes a nucleotide from any one of the 3′ Flanking Region sequences in Table 14, or a nucleotide sequence substantially identical (e.g., having at least 70%, 75%, 80%, 85%, 90%, 92%, 95%, 97%, 98%, or 99% sequence identity) thereto.

### AAV Particles Comprising Modulatory Polynucleotides

In certain embodiments, the AAV particle comprises a viral genome with a payload region comprising a modulatory polynucleotide sequence. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation or amelioration of neurological diseases and/or disorders.

In certain embodiments, the AAV particles comprising modulatory polynucleotide sequence which comprises a nucleic acid sequence encoding at least one siRNA molecule may be introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

In certain embodiments, the AAV particle viral genome may comprise at least one inverted terminal repeat (ITR) region. The length of the ITR region for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length.

In some embodiments, the AAV particle viral genome may comprises one or more inverted terminal repeat (ITR) regions. In some embodiments, the AAV particle includes one ITR region. In some embodiments, the AAV particle includes two ITR sequence regions. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

In certain embodiments, the AAV particle viral genome may comprise at least one multiple filler sequence region. As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In certain embodiments, the AAV particle viral genome may comprise at least one enhancer sequence region. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length. As a non-limiting example, the viral genome comprises an enhancer region that is about 382 nucleotides in length.

In certain embodiments, the AAV particle viral genome may comprise at least one exon sequence region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

In certain embodiments, the AAV particle viral genome may comprise at least one intron sequence region. The intron region(s) may, independently, be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In certain embodiments, the AAV particle viral genome may comprise at least one polyadenylation signal sequence region. The polyadenylation signal region sequence region(s) may, independently, have a length such as, but not limited to, about 100-600 nucleotides, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides. In some embodiments, the polyA signal region comprises a length of about 100 to 150 nucleotides, e.g., about 127 nucleotides. As a non-limiting example, the viral genome comprises a polyadenylation signal sequence region that is about 225 nucleotides in length. As a non-limiting example, the viral genome comprises a polyadenylation signal sequence region that is about 476 nucleotides in length. In some embodiments, the polyA sequence comprises a length of about 450 to 500 nucleotides, e.g., about 477 nucleotides. In some embodiments, the polyA sequence comprises a length of about 520 to about 560 nucleotides, e.g., about 552 nucleotides.

In certain embodiments, the AAV particle viral genome comprises more than one polyA signal sequence region.

Additional ITR region nucleotides, filler region nucleotides, enhancer region nucleotides, exon and intron region nucleotides, and polyA signal sequence nucleotides that may be used in the transgenes encoding the SOD1 targeting polynucleotide described herein are disclosed in PCT Patent Application Publications WO2016077687, WO2019079240, WO2018204786, WO2019079242, WO 2020010042 and WO2020223296, which are hereby incorporated by reference in their entirety

Non-limiting examples of ITR to ITR sequences of AAV particles comprising a viral genome with a payload region comprising a modulatory polynucleotide sequence are described in Table 15. Table 15 also provides an alternate name for the ITR to ITR construct indicated by the “VOYSOD” identifier.

TABLE 15
ITR to ITR Sequences of AAV Particles, H1.mir.104-788.2 (with lentivirus derived
filler) comprising Modulatory Polynucleotides

| ITR to ITR | | SEQ | |
|---|---|---|---|
| Construct | | ID | |
| Name | Region | NO | Sequence |
| H1.mir.104- | ITR-ITR | 109 | CTGCGCGCTCGCTCGCTCACTGAGGCCGCCCGGGCAAAGCCCGGG |
| 788.2 with | sequence | | CGTCGGGCGACCTTTGGTCGCCCGGCCTCAGTGAGCGAGCGAGCG |
| lentivirus | | | CGCAGAGAGGGAGTGTAGCCATGCTCTAGGAAGATCAATTCAATT |
| derived filler | | | CACGCGTCCATGGCTTAGAAGGCAAGAATCCTGGCTGTGGAAAGA |
| (VOYSOD16) | | | TACCTAAAGGATCAACAGCTCCTGGGGATTTGGGGTTGCTCTGGA |
| | | | AAACTCATTTGCACCACTGCTGTGCCTTGGAATGCTAGTTGGAGT |
| | | | AATAAATCTCTGGAACAGATTTGGAATCACACGACCTGGATGGAG |
| | | | TGGGACAGAGAAATTAACAATTACACAAGCTTAATACACTCCTTA |
| | | | ATTGAAGAATCGCAAAACCAGCAAGAAAAGAATGAACAAGAATTA |
| | | | TTGGAATTAGATAAATGGGCAAGTTTGTGGAATTGGTTTAACATA |
| | | | ACAAATTGGCTGTGGTATATAAAATTATTCATAATGATAGTAGGA |
| | | | GGCTTGGTAGGTTTAAGAATAGTTTTTGCTGTACTTTCTATAGTG |
| | | | AATAGAGTTAGGCAGGGATATTCACCATTATCGTTTCAGACCCAC |
| | | | CTCCCAACCCCGAGGGGACCCGACAGGCCCGAAGGAATAGAAGAA |
| | | | GAAGGTGGAGAGAGAGACAGAGACAGATCCATTCGATTAGTGAAC |
| | | | GGATCTCGACGGTATCGATCACGAGACTAGCCTCGAGCGGCCGCA |
| | | | ATTCGAACGCTGACGTCATCAACCCGCTCCAAGGAATCGCGGGCC |
| | | | CAGTGTCACTAGGCGGGAACACCCAGCGCGCGTGCGCCCTGGCAG |
| | | | GAAGATGGCTGTGAGGGACAGGGAGTGGCGCCCTGCAATATTTGC |
| | | | ATGTCGCTATGTGTTCTGGGAAATCACCATAAACGTGAAATGTCT |
| | | | TTGGATTTGGGAATCTTATAAGTTCTGTATGAGACCACACCGGTA |
| | | | CCGAGCTCTCCCGCAGAACACCATGCGCTCCACGGAAGCAGGTCC |
| | | | TCACTTTAATGCTGTGGCCACTGAGAAGTATTAAAGTGAGGACCT |
| | | | GCTTCTGAGGAGCGCCTTGACAGCAGCCATGGGAGGGCCTCGAGG |
| | | | ACGGGGTGAACTACGCCTGAGGATCCGATCTTTTTCCCTCTGCCA |
| | | | AAAATTATGGGGACATCATGAAGCCCCTTGAGCATCTGACTTCTG |
| | | | GCTAATAAAGGAAATTTATTTTCATTGCAATAGTGTGTTGGAATT |
| | | | TTTTGTGTCTCTCACTCGGCCTAGGTAGATAAGTAGCATGGCGGG |
| | | | TTAATCATTAACTACAAGGAACCCCTAGTGATGGAGTTGGCCACT |
| | | | CCCTCTCTGCGCGCTCGCTCGCTCACTGAGGCCGGGCGACCAAAG |
| | | | GTCGCCCGACGCCCGGGCTTTGCCCGGGCGGCCTCAGTGAGCGAG |
| | | | CGAGCGCGCAG |
| | Modulatory | 2562 | CTCCCGCAGAACACCATGCGCTCCACGGAAGCAGGTCCTCACTTT |
| | Polynucleotide | | AATGCTGTGGCCACTGAGAAGTATTAAAGTGAGGACCTGCTTCTG |
| | sequence | | AGGAGCGCCTTGACAGCAGCCATGGGAGGGCC |

Table 16 provides ITR to ITR sequence of H1.mir104-788.2 with albumin derived filler. Also provided in Table 16 are the components that comprise the ITR to ITR sequence. In some embodiments, the components may be separated from each other by vector backbone sequence. In some embodiments, the AAV particle includes the nucleotide sequence of SEQ ID NO: 109, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 109. In some embodiments, the AAV particle includes nucleotide sequence of SEQ ID NO: 2562, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2562.

TABLE 16
ITR to ITR of AAV Particles, H1.mir104-788.2 (with albumin derived filler)
comprising Modulatory Polynucleotides and its components

| | SEQ | |
|---|---|---|
| | ID | |
| Description | NO. | Sequence |
| ITR to ITR of | 125 | CTGCGCGCTCGCTCGCTCACTGAGGCCGCCCGGGCAAAGCCCGGGCGTCGGGCGACC |
| H1.mir104- | | TTTGGTCGCCCGGCCTCAGTGAGCGAGCGAGCGCGCAGAGAGGGAGTGTAGCCATGC |
| 788.2 with | | TCTAGGAAGATCAATTCAATTCACGCGTATAGTCTTCTGCACAGGGCATTCTTTTTG |
| albumin derived | | CTTCAGGATGTTTACAACATTTGCTGCCCACTTTTCCTAGGTTTCTTGAGACCTCTA |
| filler | | CAAGAGTTGGAGTTGACACTTGGGGTACTTTCTTGGTGTAACGAACTAATAGCCTGA |
| | | AAAAAAGAAGTCATGTGTTTTCAGCAAGGCAAGAAACTGTCTAACATAGTAGATAAA |
| | | ACAGAGAACACTTGGCCGGAATCAACTAAGATGTTGCTATGTTCCATTCATCATATT |
| | | ATCTCCATCTGCAGAGTAGTGGGTTAGTGGAGGGTAGAAAACATTCTCCTGAACAAC |
| | | TAGTTAAACTTGGCTTTGAGTTCCACCTGTACCACTTGCATAATCTTGGGAAAGTGA |
| | | GTTGCCTAATTCAGTGACATTAATAAATTTATTAATTTCTTCTTTCAATAAAACCTG |
| | | GAGAGAGCTTCATATGTATCAGCATATGCTAAACTTGAAAGATACAAGTAGAAAATG |
| | | GAAGGAAATATATCTGACTCAATAGGGATAGTTCAAGGGTTAAATTAAAAGTAGTAA |
| | | AGTATTATAATTAATCTGACATGGTACCCTCTAGCGGCCGCAATTCGAACGCTGACG |
| | | TCATCAACCCGCTCCAAGGAATCGCGGGCCCAGTGTCACTAGGCGGGAACACCCAGC |
| | | GCGCGTGCGCCCTGGCAGGAAGATGGCTGTGAGGGACAGGGAGTGGCGCCCTGCAAT |
| | | ATTTGCATGTCGCTATGTGTTCTGGGAAATCACCATAAACGTGAAATGTCTTTGGAT |
| | | TTGGGAATCTTATAAGTTCTGTATGAGACCACACCGGTACCGAGCTCTCCCGCAGAA |
| | | CACCATGCGCTCCACGGAAGCAGGTCCTCACTTTAATGCTGTGGCCACTGAGAAGTA |
| | | TTAAAGTGAGGACCTGCTTCTGAGGAGCGCCTTGACAGCAGCCATGGGAGGGCCTCG |
| | | AGGACGGGGTGAACTACGCCTGAGGATCCGATCTTTTTCCCTCTGCCAAAAATTATG |
| | | GGGACATCATGAAGCCCCTTGAGCATCTGACTTCTGGCTAATAAAGGAAATTTATTT |
| | | TCATTGCAATAGTGTGTTGGAATTTTTTGTGTCTCTCACTCGGCCTAGGTAGATAAG |
| | | TAGCATGGCGGGTTAATCATTAACTACAAGGAACCCCTAGTGATGGAGTTGGCCACT |
| | | CCCTCTCTGCGCGCTCGCTCGCTCACTGAGGCCGGGCGACCAAAGGTCGCCCGACGC |
| | | CCGGGCTTTGCCCGGGCGGCCTCAGTGAGCGAGCGAGCGCGCAG |

ITR-ITR Components of H1.mir104-788.2 (with albumin derived filler)

| | | |
|---|---|---|
| 5′ITR | 126 | CTGCGCGCTCGCTCGCTCACTGAGGCCGCCCGGGCAAAGCCCGGGCGTCGGGCGACC |
| | | TTTGGTCGCCCGGCCTCAGTGAGCGAGCGAGCGCGCAGAGAGGGAGTG |
| Albumin | 127 | ATAGTCTTCTGCACAGGGCATTCTTTTTGCTTCAGGATGTTTACAACATTTGCTGCC |
| derived filler | | CACTTTTCCTAGGTTTCTTGAGACCTCTACAAGAGTTGGAGTTGACACTTGGGGTAC |
| | | TTTCTTGGTGTAACGAACTAATAGCCTGAAAAAAAGAAGTCATGTGTTTTCAGCAAG |
| | | GCAAGAAACTGTCTAACATAGTAGATAAAACAGAGAACACTTGGCCGGAATCAACTA |
| | | AGATGTTGCTATGTTCCATTCATCATATTATCTCCATCTGCAGAGTAGTGGGTTAGT |
| | | GGAGGGTAGAAAACATTCTCCTGAACAACTAGTTAAACTTGGCTTTGAGTTCCACCT |
| | | GTACCACTTGCATAATCTTGGGAAAGTGAGTTGCCTAATTCAGTGACATTAATAAAT |
| | | TTATTAATTTCTTCTTTCAATAAAACCTGGAGAGAGCTTCATATGTATCAGCATATG |
| | | CTAAACTTGAAAGATACAAGTAGAAAATGGAAGGAAATATATCTGACTCAATAGGGA |
| | | TAGTTCAAGGGTTAAATTAAAAGTAGTAAAGTATTATAATTAATCTGACATGGTACC |
| H1 promoter | 128 | AATTCGAACGCTGACGTCATCAACCCGCTCCAAGGAATCGCGGGCCCAGTGTCACTA |
| | | GGCGGGAACACCCAGCGCGCGTGCGCCCTGGCAGGAAGATGGCTGTGAGGGACAGGG |
| | | AGTGGCGCCCTGCAATATTTGCATGTCGCTATGTGTTCTGGGAAATCACCATAAACG |
| | | TGAAATGTCTTTGGATTTGGGAATCTTATAAGTTCTGTATGAGACCAC |
| Modulatory | 2562 | CTCCCGCAGAACACCATGCGCTCCACGGAAGCAGGTCCTCACTTTAATGCTGTGGCC |
| Polynucleotide | | ACTGAGAAGTATTAAAGTGAGGACCTGCTTCTGAGGAGCGCCTTGACAGCAGCCATG |
| (SOD1- | | GGAGGGCC |
| miR104-788.2) | | |
| rBGpA | 129 | GATCTTTTTCCCTCTGCCAAAAATTATGGGGACATCATGAAGCCCCTTGAGCATCTG |
| | | ACTTCTGGCTAATAAAGGAAATTTATTTTCATTGCAATAGTGTGTTGGAATTTTTTG |
| | | TGTCTCTCACTCG |
| 3′ITR | 130 | AGGAACCCCTAGTGATGGAGTTGGCCACTCCCTCTCTGCGCGCTCGCTCGCTCACTG |
| | | AGGCCGGGCGACCAAAGGTCGCCCGACGCCCGGGCTTTGCCCGGGCGGCCTCAGTGA |
| | | GCGAGCGAGCGCGCAG |

Tables 17 and 18 provide further variant ITR to ITR sequences of mir104-788.2 with different promoters. In some embodiments, the components may be separated from each other by vector backbone sequence.

TABLE 17
Additional ITR to ITR sequences of AAV Particles of mir104-788.2 comprising
Modulatory Polynucleotides and their components

| | | SEQ |
|---|---|---|
| Description | Sequence | ID NO |
| ITR to ITR of | CTGCGCGCTCGCTCGCTCACTGAGGCCGCCCGGGCAAAGCCCGGGCGTCGGGCGA | 4028 |
| CBA.mir104-788.2 | CCTTTGGTCGCCCGGCCTCAGTGAGCGAGCGAGCGCGCAGAGAGGGAGTGTAGCC | |
| with hβG | ATGCTCTAGGAAGATCAATTCAATTCACGCGTCGACATTGATTATTGACTAGTTA | |
| exon/intron/exon | TTAATAGTAATCAATTACGGGGTCATTAGTTCATAGCCCATATATGGAGTTCCGC | |
| (construct 1) | GTTACATAACTTACGGTAAATGGCCCGCCTGGCTGACCGCCCAACGACCCCCGCC | |
| | CATTGACGTCAATAATGACGTATGTTCCCATAGTAACGCCAATAGGGACTTTCCA | |
| | TTGACGTCAATGGGTGGAGTATTTACGGTAAACTGCCCACTTGGCAGTACATCAA | |
| | GTGTATCATATGCCAAGTACGCCCCCTATTGACGTCAATGACGGTAAATGGCCCG | |
| | CCTGGCATTATGCCCAGTACATGACCTTATGGGACTTTCCTACTTGGCAGTACAT | |
| | CTACGTATTAGTCATCGCTATTACCATGTCGAGGCCACGTTCTGCTTCACTCTCC | |
| | CCATCTCCCCCCCCTCCCCACCCCCAATTTTGTATTTATTTATTTTTTAATTATT | |
| | TTGTGCAGCGATGGGGGCGGGGGGGGGGGGCGCGCGCCAGGCGGGGCGGGGGGGG | |
| | GCGAGGGGGGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTATAAA | |
| | AAGCGAAGCGCGCGGCGGGCGGGAGCAAGCTTCGTTTAGTGAACCGTCAGATCGC | |
| | CTGGAGACGCCATCCACGCTGTTTTGACCTCCATAGAAGACACCGGGACCGATCC | |
| | AGCCTCCGCGGATTCGAATCCCGGCCGGGAACGGTGCATTGGAACGCGGATTCCC | |
| | CGTGCCAAGAGTGACGTAAGTACCGCCTATAGAGTCTATAGGCCCACAAAAAATG | |
| | CTTTCTTCTTTTAATATACTTTTTTGTTTATCTTATTTCTAATACTTTCCCTAAT | |
| | CTCTTTCTTTCAGGGCAATAATGATACAATGTATCATGCCTCTTTGCACCATTCT | |
| | AAAGAATAACAGTGATAATTTCTGGGTTAAGGCAATAGCAATATTTCTGCATATA | |
| | AATATTTCTGCATATAAATTGTAACTGATGTAAGAGGTTTCATATTGCTAATAGC | |
| | AGCTACAATCCAGCTACCATTCTGCTTTTATTTTATGGTTGGGATAAGGCTGGAT | |
| | TATTCTGAGTCCAAGCTAGGCCCTTTTGCTAATCATGTTCATACCTCTTATCTTC | |
| | CTCCCACAGCTCCTGGGCAACGTGCTGGTCTGTGTGCTGGCCCATCACTTTGGCA | |
| | AAGAATTGGGATTCGAACCGGTACCGAGCTCTCCCGCAGAACACCATGCGCTCCA | |
| | CGGAAGCAGGTCCTCACTTTAATGCTGTGGCCACTGAGAAGTATTAAAGTGAGGA | |
| | CCTGCTTCTGAGGAGCGCCTTGACAGCAGCCATGGGAGGGCCTCGAGGGGTGGCA | |
| | TCCCTGTGACCCCTCCCCAGTGCCTCTCCTGGCCCTGGAAGTTGCCACTCCAGTG | |
| | CCCACCAGCCTTGTCCTAATAAAATTAAGTTGCATCATTTTGTCTGACTAGGTGT | |
| | CCTTCTATAATATTATGGGGTGGAGGGGGGTGGTATGGAGCAAGGGGCAAGTTGG | |
| | GAAGACAACCTGTAGGGCCTGCGGGGTCTATTGGGAACCAAGCTGGAGTGCAGTG | |
| | GCACAATCTTGGCTCACTGCAATCTCCGCCTCCTGGGTTCAAGCGATTCTCCTGC | |
| | CTCAGCCTCCCGAGTTGTTGGGATTCCAGGCATGCATGACCAGGCTCAGCTAATT | |
| | TTTGTTTTTTTGGTAGAGACGGGGTTTCACCATATTGGCCAGGCTGGTCTCCAAC | |
| | TCCTAATCTCAGGTGATCTACCCACCTTGGCCTCCCAAATTGCTGGGATTACAGG | |
| | CGTGAACCACTGCTCCCTTCCCTGTCCTTATCGATGCCTAGGGTTAATCATTAAC | |
| | TACAAGGAACCCCTAGTGATGGAGTTGGCCACTCCCTCTCTGCGCGCTCGCTCGC | |
| | TCACTGAGGCCGGGCGACCAAAGGTCGCCCGACGCCCGGGCTTTGCCCGGGCGGC | |
| | CTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4029 |
| miniCBA.mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCCACGTTCTGCTTCACTCTCCCC | |
| exon/intron/exon | ATCTCCCCCCCCTCCCCACCCCCAATTTTGTATTTATTTATTTTTTAATTATTTT | |
| (construct 2) | GTGCAGCGATGGGGGCGGGGGGGGGGGGCGCGCGCCAGGCGGGGCGGGGCGGGGC | |
| | GAGGGGCGGGGCGGGGCGAGGCGGAGAGGTGCGGCGGCAGCCAATCAGAGCGGCG | |
| | CGCTCCGAAAGTTTCCTTTTATGGCGAGGCGGCGGCGGCGGCGGCCCTATAAAAA | |
| | GCGAAGCGCGCGGCGGGAAGCTTCGTTTAGTGAACCGTCAGATCGCCTGGAGACG | |
| | CCATCCACGCTGTTTTGACCTCCATAGAAGACACCGGGACCGATCCAGCCTCCGC | |
| | GGATTCGAATCCCGGCCGGGAACGGTGCATTGGAACGCGGATTCCCCGTGCCAAG | |
| | AGTGACGTAAGTACCGCCTATAGAGTCTATAGGCCCACAAAAAATGCTTTCTTCT | |
| | TTTAATATACTTTTTTGTTTATCTTATTTCTAATACTTTCCCTAATCTCTTTCTT | |
| | TCAGGGCAATAATGATACAATGTATCATGCCTCTTTGCACCATTCTAAAGAATAA | |
| | CAGTGATAATTTCTGGGTTAAGGCAATAGCAATATTTCTGCATATAAATATTTCT | |
| | GCATATAAATTGTAACTGATGTAAGAGGTTTCATATTGCTAATAGCAGCTACAAT | |
| | CCAGCTACCATTCTGCTTTTATTTTATGGTTGGGATAAGGCTGGATTATTCTGAG | |
| | TCCAAGCTAGGCCCTTTTGCTAATCATGTTCATACCTCTTATCTTCCTCCCACAG | |
| | CTCCTGGGCAACGTGCTGGTCTGTGTGCTGGCCCATCACTTTGGCAAAGAATTGG | |
| | GATTCGAACCGGTACCGAGCTCTCCCGCAGAACACCATGCGCTCCACGGAAGCAG | |
| | GTCCTCACTTTAATGCTGTGGCCACTGAGAAGTATTAAAGTGAGGACCTGCTTCT | |
| | GAGGAGCGCCTTGACAGCAGCCATGGGAGGGCCTCGAGGGGTGGCATCCCTGTGA | |
| | CCCCTCCCCAGTGCCTCTCCTGGCCCTGGAAGTTGCCACTCCAGTGCCCACCAGC | |
| | CTTGTCCTAATAAAATTAAGTTGCATCATTTTGTCTGACTAGGTGTCCTTCTATA | |
| | ATATTATGGGGTGGAGGGGGGTGGTATGGAGCAAGGGGCAAGTTGGGAAGACAAC | |
| | CTGTAGGGCCTGCGGGGTCTATTGGGAACCAAGCTGGAGTGCAGTGGCACAATCT | |
| | TGGCTCACTGCAATCTCCGCCTCCTGGGTTCAAGCGATTCTCCTGCCTCAGCCTC | |
| | CCGAGTTGTTGGGATTCCAGGCATGCATGACCAGGCTCAGCTAATTTTTGTTTTT | |
| | TTGGTAGAGACGGGGTTTCACCATATTGGCCAGGCTGGTCTCCAACTCCTAATCT | |
| | CAGGTGATCTACCCACCTTGGCCTCCCAAATTGCTGGGATTACAGGCGTGAACCA | |
| | CTGCTCCCTTCCCTGTCCTTATCGATGCCTAGGGTTAATCATTAACTACAAGGAA | |
| | CCCCTAGTGATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGCCTCAGTGAG | |
| | CGAGCGAGCGCGCAG | |
| ITR to ITR of | CTGCGCGCTCGCTCGCTCACTGAGGCCGCCCGGGCAAAGCCCGGGCGTCGGGCGA | 4030 |
| miniEF1aV2.mir10 | CCTTTGGTCGCCCGGCCTCAGTGAGCGAGCGAGCGCGCAGAGAGGGAGTGTAGCC | |
| 4-788.2 with hBG | ATGCTCTAGGAAGATCAATTCAATTCACGCGTCGTGAGGCTCCGGTGCCCGTCAG | |
| exon/intron/exon | TGGGCAGAGCGCACATCGCCCACAGTCCCCGAGAAGTTGGGGGGAGGGGTCGGCA | |
| (construct 3) | ATTGAACCGGTGCCTAGAGAAGGTGGCGCGGGGTAAACTGGGAAAGTGATGTCGT | |
| | GTACTGGCTCCGCCTTTTTCCCGAGGGTGGGGGAGAACCGTATATAAGTGCAGTA | |
| | GTCGCCGTGAACGTTCTTTTTCGCAACGGGTTTGCCGCCAGAACACAGAAGCTTC | |
| | GTTTAGTGAACCGTCAGATCGCCTGGAGACGCCATCCACGCTGTTTTGACCTCCA | |
| | TAGAAGACACCGGGACCGATCCAGCCTCCGCGGATTCGAATCCCGGCCGGGAACG | |
| | GTGCATTGGAACGCGGATTCCCCGTGCCAAGAGTGACGTAAGTACCGCCTATAGA | |
| | GTCTATAGGCCCACAAAAAATGCTTTCTTCTTTTAATATACTTTTTTGTTTATCT | |
| | TATTTCTAATACTTTCCCTAATCTCTTTCTTTCAGGGCAATAATGATACAATGTA | |
| | TCATGCCTCTTTGCACCATTCTAAAGAATAACAGTGATAATTTCTGGGTTAAGGC | |
| | AATAGCAATATTTCTGCATATAAATATTTCTGCATATAAATTGTAACTGATGTAA | |
| | GAGGTTTCATATTGCTAATAGCAGCTACAATCCAGCTACCATTCTGCTTTTATTT | |
| | TATGGTTGGGATAAGGCTGGATTATTCTGAGTCCAAGCTAGGCCCTTTTGCTAAT | |
| | CATGTTCATACCTCTTATCTTCCTCCCACAGCTCCTGGGCAACGTGCTGGTCTGT | |
| | GTGCTGGCCCATCACTTTGGCAAAGAATTGGGATTCGAACCGGTACCGAGCTCTC | |
| | CCGCAGAACACCATGCGCTCCACGGAAGCAGGTCCTCACTTTAATGCTGTGGCCA | |
| | CTGAGAAGTATTAAAGTGAGGACCTGCTTCTGAGGAGCGCCTTGACAGCAGCCAT | |
| | GGGAGGGCCTCGAGGGGTGGCATCCCTGTGACCCCTCCCCAGTGCCTCTCCTGGC | |
| | CCTGGAAGTTGCCACTCCAGTGCCCACCAGCCTTGTCCTAATAAAATTAAGTTGC | |
| | ATCATTTTGTCTGACTAGGTGTCCTTCTATAATATTATGGGGTGGAGGGGGGTGG | |
| | TATGGAGCAAGGGGCAAGTTGGGAAGACAACCTGTAGGGCCTGCGGGGTCTATTG | |
| | GGAACCAAGCTGGAGTGCAGTGGCACAATCTTGGCTCACTGCAATCTCCGCCTCC | |
| | TGGGTTCAAGCGATTCTCCTGCCTCAGCCTCCCGAGTTGTTGGGATTCCAGGCAT | |
| | GCATGACCAGGCTCAGCTAATTTTTGTTTTTTTGGTAGAGACGGGGTTTCACCAT | |
| | ATTGGCCAGGCTGGTCTCCAACTCCTAATCTCAGGTGATCTACCCACCTTGGCCT | |
| | CCCAAATTGCTGGGATTACAGGCGTGAACCACTGCTCCCTTCCCTGTCCTTATCG | |
| | ATGCCTAGGGTTAATCATTAACTACAAGGAACCCCTAGTGATGGAGTTGGCCACT | |
| | CCCTCTCTGCGCGCTCGCTCGCTCACTGAGGCCGGGCGACCAAAGGTCGCCCGAC | |
| | GCCCGGGCTTTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4031 |
| PGK.mir104-788.2 | CCTTTGGTCGCCCGGCCTCAGTGAGCGAGCGAGCGCGCAGAGAGGGAGTGTAGCC | |
| with hBG | ATGCTCTAGGAAGATCAATTCAATTCACGCGTGGGTAGGGGAGGCGCTTTTCCCA | |
| exon/intron/exon | AGGCAGTCTGGAGCATGCGCTTTAGCAGCCCCGCTGGGCACTTGGCGCTACACAA | |
| (construct 4) | GTGGCCTCTGGCCTCGCACACATTCCACATCCACCGGTAGGCGCCAACCGGCTCC | |
| | GTTCTTTGGTGGCCCCTTCGCGCCACCTTCTACTCCTCCCCTAGTCAGGAAGTTC | |
| | CCCCCCGCCCCGCAGCTCGCGTCGTGCAGGACGTGACAAATGGAAGTAGCACGTC | |
| | TCACTAGTCTCGTGCAGATGGACAGCACCGCTGAGCAATGGAAGCGGGTAGGCCT | |
| | TTGGGGCAGCGGCCAATAGCAGCTTTGCTCCTTCGCTTTCTGGGCTCAGAGGCTG | |
| | GGAAGGGGTGGGTCCGGGGGCGGGCTCAGGGGGGGGCTCAGGGGCGGGGCGGGCG | |
| | CCCGAAGGTCCTCCGGAGGCCCGGCATTCTGCACGCTTCAAAAGCGCACGTCTGC | |
| | CGCGCTGTTCTCCTCTTCCTCATCTCCGGGCCTTTCGAAGCTTCGTTTAGTGAAC | |
| | CGTCAGATCGCCTGGAGACGCCATCCACGCTGTTTTGACCTCCATAGAAGACACC | |
| | GGGACCGATCCAGCCTCCGCGGATTCGAATCCCGGCCGGGAACGGTGCATTGGAA | |
| | CGCGGATTCCCCGTGCCAAGAGTGACGTAAGTACCGCCTATAGAGTCTATAGGCC | |
| | CACAAAAAATGCTTTCTTCTTTTAATATACTTTTTTGTTTATCTTATTTCTAATA | |
| | CTTTCCCTAATCTCTTTCTTTCAGGGCAATAATGATACAATGTATCATGCCTCTT | |
| | TGCACCATTCTAAAGAATAACAGTGATAATTTCTGGGTTAAGGCAATAGCAATAT | |
| | TTCTGCATATAAATATTTCTGCATATAAATTGTAACTGATGTAAGAGGTTTCATA | |
| | TTGCTAATAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATAC | |
| | CTCTTATCTTCCTCCCACAGCTCCTGGGCAACGTGCTGGTCTGTGTGCTGGCCCA | |
| | TCACTTTGGCAAAGAATTGGGATTCGAACCGGTACCGAGCTCTCCCGCAGAACAC | |
| | CATGCGCTCCACGGAAGCAGGTCCTCACTTTAATGCTGTGGCCACTGAGAAGTAT | |
| | TAAAGTGAGGACCTGCTTCTGAGGAGCGCCTTGACAGCAGCCATGGGAGGGCCTC | |
| | GAGGGGTGGCATCCCTGTGACCCCTCCCCAGTGCCTCTCCTGGCCCTGGAAGTTG | |
| | CCACTCCAGTGCCCACCAGCCTTGTCCTAATAAAATTAAGTTGCATCATTTTGTC | |
| | TGACTAGGTGTCCTTCTATAATATTATGGGGTGGAGGGGGGTGGTATGGAGCAAG | |
| | GGGCAAGTTGGGAAGACAACCTGTAGGGCCTGCGGGGTCTATTGGGAACCAAGCT | |
| | GGAGTGCAGTGGCACAATCTTGGCTCACTGCAATCTCCGCCTCCTGGGTTCAAGC | |
| | GATTCTCCTGCCTCAGCCTCCCGAGTTGTTGGGATTCCAGGCATGCATGACCAGG | |
| | CTCAGCTAATTTTTGTTTTTTTGGTAGAGACGGGGTTTCACCATATTGGCCAGGC | |
| | TGGTCTCCAACTCCTAATCTCAGGTGATCTACCCACCTTGGCCTCCCAAATTGCT | |
| | GGGATTACAGGCGTGAACCACTGCTCCCTTCCCTGTCCTTATCGATGCCTAGGGT | |
| | TAATCATTAACTACAAGGAACCCCTAGTGATGGAGTTGGCCACTCCCTCTCTGCG | |
| | CGCTCGCTCGCTCACTGAGGCCGGGCGACCAAAGGTCGCCCGACGCCCGGGCTTT | |
| | GCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4032 |
| MP84.mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCCCTAATGGGCCAGGCGGCAGGG | |
| exon/intron/exon | GTTGAGAGGTAGGGGAGATGGGCTCTGAGACTATAAAGCCAGCGGGGGCCCAGCA | |
| (construct 5) | GCCCTCAAGCTTCGTTTAGTGAACCGTCAGATCGCCTGGAGACGCCATCCACGCT | |
| | GTTTTGACCTCCATAGAAGACACCGGGACCGATCCAGCCTCCGCGGATTCGAATC | |
| | CCGGCCGGGAACGGTGCATTGGAACGCGGATTCCCCGTGCCAAGAGTGACGTAAG | |
| | TACCGCCTATAGAGTCTATAGGCCCACAAAAAATGCTTTCTTCTTTTAATATACT | |
| | TTTTTGTTTATCTTATTTCTAATACTTTCCCTAATCTCTTTCTTTCAGGGCAATA | |
| | ATGATACAATGTATCATGCCTCTTTGCACCATTCTAAAGAATAACAGTGATAATT | |
| | TCTGGGTTAAGGCAATAGCAATATTTCTGCATATAAATATTTCTGCATATAAATT | |
| | GTAACTGATGTAAGAGGTTTCATATTGCTAATAGCAGCTACAATCCAGCTACCAT | |
| | TCTGCTTTTATTTTATGGTTGGGATAAGGCTGGATTATTCTGAGTCCAAGCTAGG | |
| | CCCTTTTGCTAATCATGTTCATACCTCTTATCTTCCTCCCACAGCTCCTGGGCAA | |
| | CGTGCTGGTCTGTGTGCTGGCCCATCACTTTGGCAAAGAATTGGGATTCGAACCG | |
| | GTACCGAGCTCTCCCGCAGAACACCATGCGCTCCACGGAAGCAGGTCCTCACTTT | |
| | AATGCTGTGGCCACTGAGAAGTATTAAAGTGAGGACCTGCTTCTGAGGAGCGCCT | |
| | TGACAGCAGCCATGGGAGGGCCTCGAGGGGTGGCATCCCTGTGACCCCTCCCCAG | |
| | TGCCTCTCCTGGCCCTGGAAGTTGCCACTCCAGTGCCCACCAGCCTTGTCCTAAT | |
| | AAAATTAAGTTGCATCATTTTGTCTGACTAGGTGTCCTTCTATAATATTATGGGG | |
| | TGGAGGGGGGTGGTATGGAGCAAGGGGCAAGTTGGGAAGACAACCTGTAGGGCCT | |
| | GCGGGGTCTATTGGGAACCAAGCTGGAGTGCAGTGGCACAATCTTGGCTCACTGC | |
| | AATCTCCGCCTCCTGGGTTCAAGCGATTCTCCTGCCTCAGCCTCCCGAGTTGTTG | |
| | GGATTCCAGGCATGCATGACCAGGCTCAGCTAATTTTTGTTTTTTTGGTAGAGAC | |
| | GGGGTTTCACCATATTGGCCAGGCTGGTCTCCAACTCCTAATCTCAGGTGATCTA | |
| | CCCACCTTGGCCTCCCAAATTGCTGGGATTACAGGCGTGAACCACTGCTCCCTTC | |
| | CCTGTCCTTATCGATGCCTAGGGTTAATCATTAACTACAAGGAACCCCTAGTGAT | |
| | GGAGTTGGCCACTCCCTCTCTGCGCGCTCGCTCGCTCACTGAGGCCGGGCGACCA | |
| | AAGGTCGCCCGACGCCCGGGCTTTGCCCGGGCGGCCTCAGTGAGCGAGCGAGCGC | |
| | GCAG | |
| ITR to ITR of | CTGCGCGCTCGCTCGCTCACTGAGGCCGCCCGGGCAAAGCCCGGGCGTCGGGCGA | 4033 |
| hSYN(long).mir10 | CCTTTGGTCGCCCGGCCTCAGTGAGCGAGCGAGCGCGCAGAGAGGGAGTGTAGCC | |
| 4-788.2 with hBG | ATGCTCTAGGAAGATCAATTCAATTCACGCGTCGCGTGGGGTTATTTCTCTACTT | |
| exon/intron/exon | TCGTGTCTCTGAGTGTGCTTCCAGTGCCCCCCTCCCCCCAAAAAATGCCTTCTGA | |
| (construct 6) | GTTGAATATCAACACTACAAACCTAGTATCTGCAGAGGGCCCTGCGTATGAGTGC | |
| | AAGTGGGTTTTAGGACCAGGATGAGGCGGGGTGGGGGTGCCTACCTGACGACCGA | |
| | CCCCGACCCACTGGACAAGCACCCAACCCCCATTCCCCAAATTGCGCATCCCCTA | |
| | TCAGAGAGGGGGAGGGGAAACAGGATGCGGCGAGGCGCGTGCGCACTGCCAGCTT | |
| | CAGCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTCCC | |
| | GGCCACCTTGGTCGCGTCCGCGCCGCCGCCGGCCCAGCCGGACCGCACCACGCGA | |
| | GGCGCGAGATAGGGGGGCACGGGCGCGACCATCTGCGCTGCGGCGCCGGCGACTC | |
| | AGCGCTGCCTCAGTCTGCGGTGGGCAGCGGAGGAGTCGTGTCGTGCCTGAGAGCG | |
| | CAGCTGTGCTCCTGGGCACCGCGCAGTCCGCCCCCGCGGCTCCTGGCCAGACCAC | |
| | CCCTAGGACCCCCTGCCCCAAGTCGCAGCCAAGCTTCGTTTAGTGAACCGTCAGA | |
| | TCGCCTGGAGACGCCATCCACGCTGTTTTGACCTCCATAGAAGACACCGGGACCG | |
| | ATCCAGCCTCCGCGGATTCGAATCCCGGCCGGGAACGGTGCATTGGAACGCGGAT | |
| | TCCCCGTGCCAAGAGTGACGTAAGTACCGCCTATAGAGTCTATAGGCCCACAAAA | |
| | AATGCTTTCTTCTTTTAATATACTTTTTTGTTTATCTTATTTCTAATACTTTCCC | |
| | TAATCTCTTTCTTTCAGGGCAATAATGATACAATGTATCATGCCTCTTTGCACCA | |
| | TTCTAAAGAATAACAGTGATAATTTCTGGGTTAAGGCAATAGCAATATTTCTGCA | |
| | TATAAATATTTCTGCATATAAATTGTAACTGATGTAAGAGGTTTCATATTGCTAA | |
| | TAGCAGCTACAATCCAGCTACCATTCTGCTTTTATTTTATGGTTGGGATAAGGCT | |
| | GGATTATTCTGAGTCCAAGCTAGGCCCTTTTGCTAATCATGTTCATACCTCTTAT | |
| | CTTCCTCCCACAGCTCCTGGGCAACGTGCTGGTCTGTGTGCTGGCCCATCACTTT | |
| | GGCAAAGAATTGGGATTCGAACCGGTACCGAGCTCTCCCGCAGAACACCATGCGC | |
| | TCCACGGAAGCAGGTCCTCACTTTAATGCTGTGGCCACTGAGAAGTATTAAAGTG | |
| | AGGACCTGCTTCTGAGGAGCGCCTTGACAGCAGCCATGGGAGGGCCTCGAGGGGT | |
| | GGCATCCCTGTGACCCCTCCCCAGTGCCTCTCCTGGCCCTGGAAGTTGCCACTCC | |
| | AGTGCCCACCAGCCTTGTCCTAATAAAATTAAGTTGCATCATTTTGTCTGACTAG | |
| | GTGTCCTTCTATAATATTATGGGGTGGAGGGGGGTGGTATGGAGCAAGGGGCAAG | |
| | TTGGGAAGACAACCTGTAGGGCCTGCGGGGTCTATTGGGAACCAAGCTGGAGTGC | |
| | AGTGGCACAATCTTGGCTCACTGCAATCTCCGCCTCCTGGGTTCAAGCGATTCTC | |
| | CTGCCTCAGCCTCCCGAGTTGTTGGGATTCCAGGCATGCATGACCAGGCTCAGCT | |
| | AATTTTTGTTTTTTTGGTAGAGACGGGGTTTCACCATATTGGCCAGGCTGGTCTC | |
| | CAACTCCTAATCTCAGGTGATCTACCCACCTTGGCCTCCCAAATTGCTGGGATTA | |
| | CAGGCGTGAACCACTGCTCCCTTCCCTGTCCTTATCGATGCCTAGGGTTAATCAT | |
| | TAACTACAAGGAACCCCTAGTGATGGAGTTGGCCACTCCCTCTCTGCGCGCTCGC | |
| | TCGCTCACTGAGGCCGGGCGACCAAAGGTCGCCCGACGCCCGGGCTTTGCCCGGG | |
| | CGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4034 |
| hSYN(short).mir10 | CCTTTGGTCGCCCGGCCTCAGTGAGCGAGCGAGCGCGCAGAGAGGGAGTGTAGCC | |
| 4-788.2 with hBG | ATGCTCTAGGAAGATCAATTCAATTCACGCGTTAGTATCTGCAGAGGGCCCTGCG | |
| exon/intron/exon | TATGAGTGCAAGTGGGTTTTAGGACCAGGATGAGGCGGGGTGGGGGTGCCTACCT | |
| (construct 7) | GACGACCGACCCCGACCCACTGGACAAGCACCCAACCCCCATTCCCCAAATTGCG | |
| | CATCCCCTATCAGAGAGGGGGAGGGGAAACAGGATGCGGCGAGGCGCGTGCGCAC | |
| | TGCCAGCTTCAGCACCGCGGACAGTGCCTTCGCCCCCGCCTGGCGGCGCGCGCCA | |
| | CCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGC | |
| | ACCACGCGAGGCGCGAGATAGGGGGGCACGGGCGCGACCATCTGCGCTGCGGCGC | |
| | CGGCGACTCAGCGCTGCCTCAGTCTGCGGTGGGCAGCGGAGGAGTCGTGTCGTGC | |
| | CTGAGAGCGCAGCTGTGCTCCTGGGCACCGCGCAGTCCGCCCCCGCGGCTCCTGG | |
| | CCAGACCACCCCTAGGACCCCCTGCCCCAAGTCGCAGCCAAGCTTCGTTTAGTGA | |
| | ACCGTCAGATCGCCTGGAGACGCCATCCACGCTGTTTTGACCTCCATAGAAGACA | |
| | CCGGGACCGATCCAGCCTCCGCGGATTCGAATCCCGGCCGGGAACGGTGCATTGG | |
| | AACGCGGATTCCCCGTGCCAAGAGTGACGTAAGTACCGCCTATAGAGTCTATAGG | |
| | CCCACAAAAAATGCTTTCTTCTTTTAATATACTTTTTTGTTTATCTTATTTCTAA | |
| | TACTTTCCCTAATCTCTTTCTTTCAGGGCAATAATGATACAATGTATCATGCCTC | |
| | TTTGCACCATTCTAAAGAATAACAGTGATAATTTCTGGGTTAAGGCAATAGCAAT | |
| | ATTTCTGCATATAAATATTTCTGCATATAAATTGTAACTGATGTAAGAGGTTTCA | |
| | TATTGCTAATAGCAGCTACAATCCAGCTACCATTCTGCTTTTATTTTATGGTTGG | |
| | GATAAGGCTGGATTATTCTGAGTCCAAGCTAGGCCCTTTTGCTAATCATGTTCAT | |
| | ACCTCTTATCTTCCTCCCACAGCTCCTGGGCAACGTGCTGGTCTGTGTGCTGGCC | |
| | CATCACTTTGGCAAAGAATTGGGATTCGAACCGGTACCGAGCTCTCCCGCAGAAC | |
| | ACCATGCGCTCCACGGAAGCAGGTCCTCACTTTAATGCTGTGGCCACTGAGAAGT | |
| | ATTAAAGTGAGGACCTGCTTCTGAGGAGCGCCTTGACAGCAGCCATGGGAGGGCC | |
| | TCGAGGGGTGGCATCCCTGTGACCCCTCCCCAGTGCCTCTCCTGGCCCTGGAAGT | |
| | TGCCACTCCAGTGCCCACCAGCCTTGTCCTAATAAAATTAAGTTGCATCATTTTG | |
| | TCTGACTAGGTGTCCTTCTATAATATTATGGGGTGGAGGGGGGTGGTATGGAGCA | |
| | AGGGGCAAGTTGGGAAGACAACCTGTAGGGCCTGCGGGGTCTATTGGGAACCAAG | |
| | CTGGAGTGCAGTGGCACAATCTTGGCTCACTGCAATCTCCGCCTCCTGGGTTCAA | |
| | GCGATTCTCCTGCCTCAGCCTCCCGAGTTGTTGGGATTCCAGGCATGCATGACCA | |
| | GGCTCAGCTAATTTTTGTTTTTTTGGTAGAGACGGGGTTTCACCATATTGGCCAG | |
| | GCTGGTCTCCAACTCCTAATCTCAGGTGATCTACCCACCTTGGCCTCCCAAATTG | |
| | CTGGGATTACAGGCGTGAACCACTGCTCCCTTCCCTGTCCTTATCGATGCCTAGG | |
| | GTTAATCATTAACTACAAGGAACCCCTAGTGATGGAGTTGGCCACTCCCTCTCTG | |
| | CGCGCTCGCTCGCTCACTGAGGCCGGGCGACCAAAGGTCGCCCGACGCCCGGGCT | |
| | TTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4035 |
| CBA.mir104-788.2 | CCTTTGGTCGCCCGGCCTCAGTGAGCGAGCGAGCGCGCAGAGAGGGAGTGTAGCC | |
| with CpG deleted | ATGCTCTAGGAAGATCAATTCAATTCACGCGTCGACATTGATTATTGACTAGTTA | |
| hBG | TTAATAGTAATCAATTACGGGGTCATTAGTTCATAGCCCATATATGGAGTTCCGC | |
| exon/intron/exon | GTTACATAACTTACGGTAAATGGCCCGCCTGGCTGACCGCCCAACGACCCCCGCC | |
| (construct 8) | CATTGACGTCAATAATGACGTATGTTCCCATAGTAACGCCAATAGGGACTTTCCA | |
| | TTGACGTCAATGGGTGGAGTATTTACGGTAAACTGCCCACTTGGCAGTACATCAA | |
| | GTGTATCATATGCCAAGTACGCCCCCTATTGACGTCAATGACGGTAAATGGCCCG | |
| | CCTGGCATTATGCCCAGTACATGACCTTATGGGACTTTCCTACTTGGCAGTACAT | |
| | CTACGTATTAGTCATCGCTATTACCATGTCGAGGCCACGTTCTGCTTCACTCTCC | |
| | CCATCTCCCCCCCCTCCCCACCCCCAATTTTGTATTTATTTATTTTTTAATTATT | |
| | TTGTGCAGCGATGGGGGCGGGGGGGGGGGGCGCGCGCCAGGCGGGGCGGGGCGGG | |
| | GCGAGGGGGGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTATAAA | |
| | AAGCGAAGCGCGCGGCGGGCGGGAGCAAGCTTCGTTTAGTGAACCGTCAGATCAC | |
| | CTGGAGACACCATCCACACTGTTTTGACCTCCATAGAAGACACCAGGACCAATCC | |
| | AGCCTCCACAGATTCCAATCCCAGCCAGGAACAGTGCATTGGAACACAGATTCCC | |
| | CTTGCCAAGAGTGACGTAAGTACCGCCTATAGAGTCTATAGGCCCACAAAAAATG | |
| | CTTTCTTCTTTTAATATACTTTTTTGTTTATCTTATTTCTAATACTTTCCCTAAT | |
| | CTCTTTCTTTCAGGGCAATAATGATACAATGTATCATGCCTCTTTGCACCATTCT | |
| | AAAGAATAACAGTGATAATTTCTGGGTTAAGGCAATAGCAATATTTCTGCATATA | |
| | AATATTTCTGCATATAAATTGTAACTGATGTAAGAGGTTTCATATTGCTAATAGC | |
| | AGCTACAATCCAGCTACCATTCTGCTTTTATTTTATGGTTGGGATAAGGCTGGAT | |
| | TATTCTGAGTCCAAGCTAGGCCCTTTTGCTAATCATGTTCATACCTCTTATCTTC | |
| | CTCCCACAGCTCCTGGGCAACCTGCTGGTCTGTGTGCTGGCCCATCACTTTGGCA | |
| | AAGAATTGGGATTCGAACCGGTACCGAGCTCTCCCGCAGAACACCATGCGCTCCA | |
| | CGGAAGCAGGTCCTCACTTTAATGCTGTGGCCACTGAGAAGTATTAAAGTGAGGA | |
| | CCTGCTTCTGAGGAGCGCCTTGACAGCAGCCATGGGAGGGCCTCGAGGGGTGGCA | |
| | TCCCTGTGACCCCTCCCCAGTGCCTCTCCTGGCCCTGGAAGTTGCCACTCCAGTG | |
| | CCCACCAGCCTTGTCCTAATAAAATTAAGTTGCATCATTTTGTCTGACTAGGTGT | |
| | CCTTCTATAATATTATGGGGTGGAGGGGGGTGGTATGGAGCAAGGGGCAAGTTGG | |
| | GAAGACAACCTGTAGGGCCTGCGGGGTCTATTGGGAACCAAGCTGGAGTGCAGTG | |
| | GCACAATCTTGGCTCACTGCAATCTCCGCCTCCTGGGTTCAAGCGATTCTCCTGC | |
| | CTCAGCCTCCCGAGTTGTTGGGATTCCAGGCATGCATGACCAGGCTCAGCTAATT | |
| | TTTGTTTTTTTGGTAGAGACGGGGTTTCACCATATTGGCCAGGCTGGTCTCCAAC | |
| | TCCTAATCTCAGGTGATCTACCCACCTTGGCCTCCCAAATTGCTGGGATTACAGG | |
| | CGTGAACCACTGCTCCCTTCCCTGTCCTTATCGATGCCTAGGGTTAATCATTAAC | |
| | TACAAGGAACCCCTAGTGATGGAGTTGGCCACTCCCTCTCTGCGCGCTCGCTCGC | |
| | TCACTGAGGCCGGGCGACCAAAGGTCGCCCGACGCCCGGGCTTTGCCCGGGCGGC | |
| | CTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4036 |
| miniCBA.mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCCACGTTCTGCTTCACTCTCCCC | |
| deleted hBG | ATCTCCCCCCCCTCCCCACCCCCAATTTTGTATTTATTTATTTTTTAATTATTTT | |
| exon/intron/exon | GTGCAGCGATGGGGGCGGGGGGGGGGGGCGCGCGCCAGGCGGGGCGGGGCGGGGC | |
| (construct 9) | GAGGGGCGGGGCGGGGCGAGGCGGAGAGGTGCGGCGGCAGCCAATCAGAGCGGCG | |
| | CGCTCCGAAAGTTTCCTTTTATGGCGAGGCGGCGGCGGCGGCGGCCCTATAAAAA | |
| | GCGAAGCGCGCGGCGGGAAGCTTCGTTTAGTGAACCGTCAGATCACCTGGAGACA | |
| | CCATCCACACTGTTTTGACCTCCATAGAAGACACCAGGACCAATCCAGCCTCCAC | |
| | AGATTCCAATCCCAGCCAGGAACAGTGCATTGGAACACAGATTCCCCTTGCCAAG | |
| | AGTGACGTAAGTACCGCCTATAGAGTCTATAGGCCCACAAAAAATGCTTTCTTCT | |
| | TTTAATATACTTTTTTGTTTATCTTATTTCTAATACTTTCCCTAATCTCTTTCTT | |
| | TCAGGGCAATAATGATACAATGTATCATGCCTCTTTGCACCATTCTAAAGAATAA | |
| | CAGTGATAATTTCTGGGTTAAGGCAATAGCAATATTTCTGCATATAAATATTTCT | |
| | GCATATAAATTGTAACTGATGTAAGAGGTTTCATATTGCTAATAGCAGCTACAAT | |
| | CCAGCTACCATTCTGCTTTTATTTTATGGTTGGGATAAGGCTGGATTATTCTGAG | |
| | TCCAAGCTAGGCCCTTTTGCTAATCATGTTCATACCTCTTATCTTCCTCCCACAG | |
| | CTCCTGGGCAACCTGCTGGTCTGTGTGCTGGCCCATCACTTTGGCAAAGAATTGG | |
| | GATTCGAACCGGTACCGAGCTCTCCCGCAGAACACCATGCGCTCCACGGAAGCAG | |
| | GTCCTCACTTTAATGCTGTGGCCACTGAGAAGTATTAAAGTGAGGACCTGCTTCT | |
| | GAGGAGCGCCTTGACAGCAGCCATGGGAGGGCCTCGAGGGGTGGCATCCCTGTGA | |
| | CCCCTCCCCAGTGCCTCTCCTGGCCCTGGAAGTTGCCACTCCAGTGCCCACCAGC | |
| | CTTGTCCTAATAAAATTAAGTTGCATCATTTTGTCTGACTAGGTGTCCTTCTATA | |
| | ATATTATGGGGTGGAGGGGGGTGGTATGGAGCAAGGGGCAAGTTGGGAAGACAAC | |
| | CTGTAGGGCCTGCGGGGTCTATTGGGAACCAAGCTGGAGTGCAGTGGCACAATCT | |
| | TGGCTCACTGCAATCTCCGCCTCCTGGGTTCAAGCGATTCTCCTGCCTCAGCCTC | |
| | CCGAGTTGTTGGGATTCCAGGCATGCATGACCAGGCTCAGCTAATTTTTGTTTTT | |
| | TTGGTAGAGACGGGGTTTCACCATATTGGCCAGGCTGGTCTCCAACTCCTAATCT | |
| | CAGGTGATCTACCCACCTTGGCCTCCCAAATTGCTGGGATTACAGGCGTGAACCA | |
| | CTGCTCCCTTCCCTGTCCTTATCGATGCCTAGGGTTAATCATTAACTACAAGGAA | |
| | CCCCTAGTGATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGCCTCAGTGAG | |
| | CGAGCGAGCGCGCAG | |
| ITR to ITR of | CTGCGCGCTCGCTCGCTCACTGAGGCCGCCCGGGCAAAGCCCGGGCGTCGGGCGA | 4037 |
| miniEF1aV2.mir10 | CCTTTGGTCGCCCGGCCTCAGTGAGCGAGCGAGCGCGCAGAGAGGGAGTGTAGCC | |
| 4-788.2 with CpG | ATGCTCTAGGAAGATCAATTCAATTCACGCGTCGTGAGGCTCCGGTGCCCGTCAG | |
| deleted hBG | TGGGCAGAGCGCACATCGCCCACAGTCCCCGAGAAGTTGGGGGGAGGGGTCGGCA | |
| exon/intron/exon | ATTGAACCGGTGCCTAGAGAAGGTGGCGCGGGGTAAACTGGGAAAGTGATGTCGT | |
| (construct 10) | GTACTGGCTCCGCCTTTTTCCCGAGGGTGGGGGAGAACCGTATATAAGTGCAGTA | |
| | GTCGCCGTGAACGTTCTTTTTCGCAACGGGTTTGCCGCCAGAACACAGAAGCTTC | |
| | GTTTAGTGAACCGTCAGATCACCTGGAGACACCATCCACACTGTTTTGACCTCCA | |
| | TAGAAGACACCAGGACCAATCCAGCCTCCACAGATTCCAATCCCAGCCAGGAACA | |
| | GTGCATTGGAACACAGATTCCCCTTGCCAAGAGTGACGTAAGTACCGCCTATAGA | |
| | GTCTATAGGCCCACAAAAAATGCTTTCTTCTTTTAATATACTTTTTTGTTTATCT | |
| | TATTTCTAATACTTTCCCTAATCTCTTTCTTTCAGGGCAATAATGATACAATGTA | |
| | TCATGCCTCTTTGCACCATTCTAAAGAATAACAGTGATAATTTCTGGGTTAAGGC | |
| | AATAGCAATATTTCTGCATATAAATATTTCTGCATATAAATTGTAACTGATGTAA | |
| | GAGGTTTCATATTGCTAATAGCAGCTACAATCCAGCTACCATTCTGCTTTTATTT | |
| | TATGGTTGGGATAAGGCTGGATTATTCTGAGTCCAAGCTAGGCCCTTTTGCTAAT | |
| | CATGTTCATACCTCTTATCTTCCTCCCACAGCTCCTGGGCAACCTGCTGGTCTGT | |
| | GTGCTGGCCCATCACTTTGGCAAAGAATTGGGATTCGAACCGGTACCGAGCTCTC | |
| | CCGCAGAACACCATGCGCTCCACGGAAGCAGGTCCTCACTTTAATGCTGTGGCCA | |
| | CTGAGAAGTATTAAAGTGAGGACCTGCTTCTGAGGAGCGCCTTGACAGCAGCCAT | |
| | GGGAGGGCCTCGAGGGGTGGCATCCCTGTGACCCCTCCCCAGTGCCTCTCCTGGC | |
| | CCTGGAAGTTGCCACTCCAGTGCCCACCAGCCTTGTCCTAATAAAATTAAGTTGC | |
| | ATCATTTTGTCTGACTAGGTGTCCTTCTATAATATTATGGGGTGGAGGGGGGTGG | |
| | TATGGAGCAAGGGGCAAGTTGGGAAGACAACCTGTAGGGCCTGCGGGGTCTATTG | |
| | GGAACCAAGCTGGAGTGCAGTGGCACAATCTTGGCTCACTGCAATCTCCGCCTCC | |
| | TGGGTTCAAGCGATTCTCCTGCCTCAGCCTCCCGAGTTGTTGGGATTCCAGGCAT | |
| | GCATGACCAGGCTCAGCTAATTTTTGTTTTTTTGGTAGAGACGGGGTTTCACCAT | |
| | ATTGGCCAGGCTGGTCTCCAACTCCTAATCTCAGGTGATCTACCCACCTTGGCCT | |
| | CCCAAATTGCTGGGATTACAGGCGTGAACCACTGCTCCCTTCCCTGTCCTTATCG | |
| | ATGCCTAGGGTTAATCATTAACTACAAGGAACCCCTAGTGATGGAGTTGGCCACT | |
| | CCCTCTCTGCGCGCTCGCTCGCTCACTGAGGCCGGGCGACCAAAGGTCGCCCGAC | |
| | GCCCGGGCTTTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4038 |
| PGK.mir104-788.2 | CCTTTGGTCGCCCGGCCTCAGTGAGCGAGCGAGCGCGCAGAGAGGGAGTGTAGCC | |
| with CpG deleted | ATGCTCTAGGAAGATCAATTCAATTCACGCGTGGGTAGGGGAGGCGCTTTTCCCA | |
| hBG | AGGCAGTCTGGAGCATGCGCTTTAGCAGCCCCGCTGGGCACTTGGCGCTACACAA | |
| exon/intron/exon | GTGGCCTCTGGCCTCGCACACATTCCACATCCACCGGTAGGCGCCAACCGGCTCC | |
| (construct 11) | GTTCTTTGGTGGCCCCTTCGCGCCACCTTCTACTCCTCCCCTAGTCAGGAAGTTC | |
| | CCCCCCGCCCCGCAGCTCGCGTCGTGCAGGACGTGACAAATGGAAGTAGCACGTC | |
| | TCACTAGTCTCGTGCAGATGGACAGCACCGCTGAGCAATGGAAGCGGGTAGGCCT | |
| | TTGGGGCAGCGGCCAATAGCAGCTTTGCTCCTTCGCTTTCTGGGCTCAGAGGCTG | |
| | GGAAGGGGTGGGTCCGGGGGCGGGCTCAGGGGCGGGCTCAGGGGCGGGGCGGGCG | |
| | CCCGAAGGTCCTCCGGAGGCCCGGCATTCTGCACGCTTCAAAAGCGCACGTCTGC | |
| | CGCGCTGTTCTCCTCTTCCTCATCTCCGGGCCTTTCGAAGCTTCGTTTAGTGAAC | |
| | CGTCAGATCACCTGGAGACACCATCCACACTGTTTTGACCTCCATAGAAGACACC | |
| | AGGACCAATCCAGCCTCCACAGATTCCAATCCCAGCCAGGAACAGTGCATTGGAA | |
| | CACAGATTCCCCTTGCCAAGAGTGACGTAAGTACCGCCTATAGAGTCTATAGGCC | |
| | CACAAAAAATGCTTTCTTCTTTTAATATACTTTTTTGTTTATCTTATTTCTAATA | |
| | CTTTCCCTAATCTCTTTCTTTCAGGGCAATAATGATACAATGTATCATGCCTCTT | |
| | TGCACCATTCTAAAGAATAACAGTGATAATTTCTGGGTTAAGGCAATAGCAATAT | |
| | TTCTGCATATAAATATTTCTGCATATAAATTGTAACTGATGTAAGAGGTTTCATA | |
| | TTGCTAATAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATAC | |
| | CTCTTATCTTCCTCCCACAGCTCCTGGGCAACCTGCTGGTCTGTGTGCTGGCCCA | |
| | TCACTTTGGCAAAGAATTGGGATTCGAACCGGTACCGAGCTCTCCCGCAGAACAC | |
| | CATGCGCTCCACGGAAGCAGGTCCTCACTTTAATGCTGTGGCCACTGAGAAGTAT | |
| | TAAAGTGAGGACCTGCTTCTGAGGAGCGCCTTGACAGCAGCCATGGGAGGGCCTC | |
| | GAGGGGTGGCATCCCTGTGACCCCTCCCCAGTGCCTCTCCTGGCCCTGGAAGTTG | |
| | CCACTCCAGTGCCCACCAGCCTTGTCCTAATAAAATTAAGTTGCATCATTTTGTC | |
| | TGACTAGGTGTCCTTCTATAATATTATGGGGTGGAGGGGGGTGGTATGGAGCAAG | |
| | GGGCAAGTTGGGAAGACAACCTGTAGGGCCTGCGGGGTCTATTGGGAACCAAGCT | |
| | GGAGTGCAGTGGCACAATCTTGGCTCACTGCAATCTCCGCCTCCTGGGTTCAAGC | |
| | GATTCTCCTGCCTCAGCCTCCCGAGTTGTTGGGATTCCAGGCATGCATGACCAGG | |
| | CTCAGCTAATTTTTGTTTTTTTGGTAGAGACGGGGTTTCACCATATTGGCCAGGC | |
| | TGGTCTCCAACTCCTAATCTCAGGTGATCTACCCACCTTGGCCTCCCAAATTGCT | |
| | GGGATTACAGGCGTGAACCACTGCTCCCTTCCCTGTCCTTATCGATGCCTAGGGT | |
| | TAATCATTAACTACAAGGAACCCCTAGTGATGGAGTTGGCCACTCCCTCTCTGCG | |
| | CGCTCGCTCGCTCACTGAGGCCGGGCGACCAAAGGTCGCCCGACGCCCGGGCTTT | |
| | GCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4039 |
| MP84.mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCCCTAATGGGCCAGGCGGCAGGG | |
| deleted hBG | GTTGAGAGGTAGGGGAGATGGGCTCTGAGACTATAAAGCCAGCGGGGGCCCAGCA | |
| exon/intron/exon | GCCCTCAAGCTTCGTTTAGTGAACCGTCAGATCACCTGGAGACACCATCCACACT | |
| (construct 12) | GTTTTGACCTCCATAGAAGACACCAGGACCAATCCAGCCTCCACAGATTCCAATC | |
| | CCAGCCAGGAACAGTGCATTGGAACACAGATTCCCCTTGCCAAGAGTGACGTAAG | |
| | TACCGCCTATAGAGTCTATAGGCCCACAAAAAATGCTTTCTTCTTTTAATATACT | |
| | TTTTTGTTTATCTTATTTCTAATACTTTCCCTAATCTCTTTCTTTCAGGGCAATA | |
| | ATGATACAATGTATCATGCCTCTTTGCACCATTCTAAAGAATAACAGTGATAATT | |
| | TCTGGGTTAAGGCAATAGCAATATTTCTGCATATAAATATTTCTGCATATAAATT | |
| | GTAACTGATGTAAGAGGTTTCATATTGCTAATAGCAGCTACAATCCAGCTACCAT | |
| | TCTGCTTTTATTTTATGGTTGGGATAAGGCTGGATTATTCTGAGTCCAAGCTAGG | |
| | CCCTTTTGCTAATCATGTTCATACCTCTTATCTTCCTCCCACAGCTCCTGGGCAA | |
| | CCTGCTGGTCTGTGTGCTGGCCCATCACTTTGGCAAAGAATTGGGATTCGAACCG | |
| | GTACCGAGCTCTCCCGCAGAACACCATGCGCTCCACGGAAGCAGGTCCTCACTTT | |
| | AATGCTGTGGCCACTGAGAAGTATTAAAGTGAGGACCTGCTTCTGAGGAGCGCCT | |
| | TGACAGCAGCCATGGGAGGGCCTCGAGGGGTGGCATCCCTGTGACCCCTCCCCAG | |
| | TGCCTCTCCTGGCCCTGGAAGTTGCCACTCCAGTGCCCACCAGCCTTGTCCTAAT | |
| | AAAATTAAGTTGCATCATTTTGTCTGACTAGGTGTCCTTCTATAATATTATGGGG | |
| | TGGAGGGGGGTGGTATGGAGCAAGGGGCAAGTTGGGAAGACAACCTGTAGGGCCT | |
| | GCGGGGTCTATTGGGAACCAAGCTGGAGTGCAGTGGCACAATCTTGGCTCACTGC | |
| | AATCTCCGCCTCCTGGGTTCAAGCGATTCTCCTGCCTCAGCCTCCCGAGTTGTTG | |
| | GGATTCCAGGCATGCATGACCAGGCTCAGCTAATTTTTGTTTTTTTGGTAGAGAC | |
| | GGGGTTTCACCATATTGGCCAGGCTGGTCTCCAACTCCTAATCTCAGGTGATCTA | |
| | CCCACCTTGGCCTCCCAAATTGCTGGGATTACAGGCGTGAACCACTGCTCCCTTC | |
| | CCTGTCCTTATCGATGCCTAGGGTTAATCATTAACTACAAGGAACCCCTAGTGAT | |
| | GGAGTTGGCCACTCCCTCTCTGCGCGCTCGCTCGCTCACTGAGGCCGGGCGACCA | |
| | AAGGTCGCCCGACGCCCGGGCTTTGCCCGGGCGGCCTCAGTGAGCGAGCGAGCGC | |
| | GCAG | |
| ITR to ITR of | CTGCGCGCTCGCTCGCTCACTGAGGCCGCCCGGGCAAAGCCCGGGCGTCGGGCGA | 4040 |
| hSYN(long).mir10 | CCTTTGGTCGCCCGGCCTCAGTGAGCGAGCGAGCGCGCAGAGAGGGAGTGTAGCC | |
| 4-788.2 with CpG | ATGCTCTAGGAAGATCAATTCAATTCACGCGTCGCGTGGGGTTATTTCTCTACTT | |
| deleted hBG | TCGTGTCTCTGAGTGTGCTTCCAGTGCCCCCCTCCCCCCAAAAAATGCCTTCTGA | |
| exon/intron/exon | GTTGAATATCAACACTACAAACCTAGTATCTGCAGAGGGCCCTGCGTATGAGTGC | |
| (construct 13) | AAGTGGGTTTTAGGACCAGGATGAGGCGGGGTGGGGGTGCCTACCTGACGACCGA | |
| | CCCCGACCCACTGGACAAGCACCCAACCCCCATTCCCCAAATTGCGCATCCCCTA | |
| | TCAGAGAGGGGGAGGGGAAACAGGATGCGGCGAGGCGCGTGCGCACTGCCAGCTT | |
| | CAGCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTCCC | |
| | GGCCACCTTGGTCGCGTCCGCGCCGCCGCCGGCCCAGCCGGACCGCACCACGCGA | |
| | GGCGCGAGATAGGGGGGCACGGGCGCGACCATCTGCGCTGCGGCGCCGGCGACTC | |
| | AGCGCTGCCTCAGTCTGCGGTGGGCAGCGGAGGAGTCGTGTCGTGCCTGAGAGCG | |
| | CAGCTGTGCTCCTGGGCACCGCGCAGTCCGCCCCCGCGGCTCCTGGCCAGACCAC | |
| | CCCTAGGACCCCCTGCCCCAAGTCGCAGCCAAGCTTCGTTTAGTGAACCGTCAGA | |
| | TCACCTGGAGACACCATCCACACTGTTTTGACCTCCATAGAAGACACCAGGACCA | |
| | ATCCAGCCTCCACAGATTCCAATCCCAGCCAGGAACAGTGCATTGGAACACAGAT | |
| | TCCCCTTGCCAAGAGTGACGTAAGTACCGCCTATAGAGTCTATAGGCCCACAAAA | |
| | AATGCTTTCTTCTTTTAATATACTTTTTTGTTTATCTTATTTCTAATACTTTCCC | |
| | TAATCTCTTTCTTTCAGGGCAATAATGATACAATGTATCATGCCTCTTTGCACCA | |
| | TTCTAAAGAATAACAGTGATAATTTCTGGGTTAAGGCAATAGCAATATTTCTGCA | |
| | TATAAATATTTCTGCATATAAATTGTAACTGATGTAAGAGGTTTCATATTGCTAA | |
| | TAGCAGCTACAATCCAGCTACCATTCTGCTTTTATTTTATGGTTGGGATAAGGCT | |
| | GGATTATTCTGAGTCCAAGCTAGGCCCTTTTGCTAATCATGTTCATACCTCTTAT | |
| | CTTCCTCCCACAGCTCCTGGGCAACCTGCTGGTCTGTGTGCTGGCCCATCACTTT | |
| | GGCAAAGAATTGGGATTCGAACCGGTACCGAGCTCTCCCGCAGAACACCATGCGC | |
| | TCCACGGAAGCAGGTCCTCACTTTAATGCTGTGGCCACTGAGAAGTATTAAAGTG | |
| | AGGACCTGCTTCTGAGGAGCGCCTTGACAGCAGCCATGGGAGGGCCTCGAGGGGT | |
| | GGCATCCCTGTGACCCCTCCCCAGTGCCTCTCCTGGCCCTGGAAGTTGCCACTCC | |
| | AGTGCCCACCAGCCTTGTCCTAATAAAATTAAGTTGCATCATTTTGTCTGACTAG | |
| | GTGTCCTTCTATAATATTATGGGGTGGAGGGGGGTGGTATGGAGCAAGGGGCAAG | |
| | TTGGGAAGACAACCTGTAGGGCCTGCGGGGTCTATTGGGAACCAAGCTGGAGTGC | |
| | AGTGGCACAATCTTGGCTCACTGCAATCTCCGCCTCCTGGGTTCAAGCGATTCTC | |
| | CTGCCTCAGCCTCCCGAGTTGTTGGGATTCCAGGCATGCATGACCAGGCTCAGCT | |
| | AATTTTTGTTTTTTTGGTAGAGACGGGGTTTCACCATATTGGCCAGGCTGGTCTC | |
| | CAACTCCTAATCTCAGGTGATCTACCCACCTTGGCCTCCCAAATTGCTGGGATTA | |
| | CAGGCGTGAACCACTGCTCCCTTCCCTGTCCTTATCGATGCCTAGGGTTAATCAT | |
| | TAACTACAAGGAACCCCTAGTGATGGAGTTGGCCACTCCCTCTCTGCGCGCTCGC | |
| | TCGCTCACTGAGGCCGGGCGACCAAAGGTCGCCCGACGCCCGGGCTTTGCCCGGG | |
| | CGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4041 |
| hSYN(short).mir10 | CCTTTGGTCGCCCGGCCTCAGTGAGCGAGCGAGCGCGCAGAGAGGGAGTGTAGCC | |
| 4-788.2 with CpG | ATGCTCTAGGAAGATCAATTCAATTCACGCGTTAGTATCTGCAGAGGGCCCTGCG | |
| deleted hBG | TATGAGTGCAAGTGGGTTTTAGGACCAGGATGAGGCGGGGTGGGGGTGCCTACCT | |
| exon/intron/exon | GACGACCGACCCCGACCCACTGGACAAGCACCCAACCCCCATTCCCCAAATTGCG | |
| (construct 14) | CATCCCCTATCAGAGAGGGGGAGGGGAAACAGGATGCGGCGAGGCGCGTGCGCAC | |
| | TGCCAGCTTCAGCACCGCGGACAGTGCCTTCGCCCCCGCCTGGCGGCGCGCGCCA | |
| | CCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGC | |
| | ACCACGCGAGGCGCGAGATAGGGGGGCACGGGCGCGACCATCTGCGCTGCGGCGC | |
| | CGGCGACTCAGCGCTGCCTCAGTCTGCGGTGGGCAGCGGAGGAGTCGTGTCGTGC | |
| | CTGAGAGCGCAGCTGTGCTCCTGGGCACCGCGCAGTCCGCCCCCGCGGCTCCTGG | |
| | CCAGACCACCCCTAGGACCCCCTGCCCCAAGTCGCAGCCAAGCTTCGTTTAGTGA | |
| | ACCGTCAGATCACCTGGAGACACCATCCACACTGTTTTGACCTCCATAGAAGACA | |
| | CCAGGACCAATCCAGCCTCCACAGATTCCAATCCCAGCCAGGAACAGTGCATTGG | |
| | AACACAGATTCCCCTTGCCAAGAGTGACGTAAGTACCGCCTATAGAGTCTATAGG | |
| | CCCACAAAAAATGCTTTCTTCTTTTAATATACTTTTTTGTTTATCTTATTTCTAA | |
| | TACTTTCCCTAATCTCTTTCTTTCAGGGCAATAATGATACAATGTATCATGCCTC | |
| | TTTGCACCATTCTAAAGAATAACAGTGATAATTTCTGGGTTAAGGCAATAGCAAT | |
| | ATTTCTGCATATAAATATTTCTGCATATAAATTGTAACTGATGTAAGAGGTTTCA | |
| | TATTGCTAATAGCAGCTACAATCCAGCTACCATTCTGCTTTTATTTTATGGTTGG | |
| | GATAAGGCTGGATTATTCTGAGTCCAAGCTAGGCCCTTTTGCTAATCATGTTCAT | |
| | ACCTCTTATCTTCCTCCCACAGCTCCTGGGCAACCTGCTGGTCTGTGTGCTGGCC | |
| | CATCACTTTGGCAAAGAATTGGGATTCGAACCGGTACCGAGCTCTCCCGCAGAAC | |
| | ACCATGCGCTCCACGGAAGCAGGTCCTCACTTTAATGCTGTGGCCACTGAGAAGT | |
| | ATTAAAGTGAGGACCTGCTTCTGAGGAGCGCCTTGACAGCAGCCATGGGAGGGCC | |
| | TCGAGGGGTGGCATCCCTGTGACCCCTCCCCAGTGCCTCTCCTGGCCCTGGAAGT | |
| | TGCCACTCCAGTGCCCACCAGCCTTGTCCTAATAAAATTAAGTTGCATCATTTTG | |
| | TCTGACTAGGTGTCCTTCTATAATATTATGGGGTGGAGGGGGGTGGTATGGAGCA | |
| | AGGGGCAAGTTGGGAAGACAACCTGTAGGGCCTGCGGGGTCTATTGGGAACCAAG | |
| | CTGGAGTGCAGTGGCACAATCTTGGCTCACTGCAATCTCCGCCTCCTGGGTTCAA | |
| | GCGATTCTCCTGCCTCAGCCTCCCGAGTTGTTGGGATTCCAGGCATGCATGACCA | |
| | GGCTCAGCTAATTTTTGTTTTTTTGGTAGAGACGGGGTTTCACCATATTGGCCAG | |
| | GCTGGTCTCCAACTCCTAATCTCAGGTGATCTACCCACCTTGGCCTCCCAAATTG | |
| | CTGGGATTACAGGCGTGAACCACTGCTCCCTTCCCTGTCCTTATCGATGCCTAGG | |
| | GTTAATCATTAACTACAAGGAACCCCTAGTGATGGAGTTGGCCACTCCCTCTCTG | |
| | CGCGCTCGCTCGCTCACTGAGGCCGGGCGACCAAAGGTCGCCCGACGCCCGGGCT | |
| | TTGCCCGGGCGGCCTCAGTGAGCGAGCGAGCGCGCAG | |

TABLE 18
Summary of components of the ITR to ITR sequences of AAV Particles in Table 17

| Construct | | | | | hGH poly | |
|---|---|---|---|---|---|---|
| (from | | | hβG | Modulatory | A signal | |
| Table 17) | 5′ITR | Promoter | Exon/Intron/Exon | polynucleotide | sequence | 3′ITR |
| 1 | SEQ ID: | SEQ ID: | SEQ ID NO: 4042 | SEQ ID NO: | SEQ ID | SEQ ID: |
| (SEQ ID | 126 | 4021 | | 2562 | NO: 4027 | 130 |
| NO: 4028) | | | | | | |
| 2 | SEQ ID: | SEQ ID: | SEQ ID NO: 4042 | SEQ ID NO: | SEQ ID | SEQ ID: |
| (SEQ ID | 126 | 4022 | | 2562 | NO: 4027 | 130 |
| NO: 4029) | | | | | | |
| 3 | SEQ ID: | SEQ ID: | SEQ ID NO: 4042 | SEQ ID NO: | SEQ ID | SEQ ID: |
| (SEQ ID | 126 | 4014 | | 2562 | NO: 4027 | 130 |
| NO: 4030) | | | | | | |
| 4 | SEQ ID: | SEQ ID: | SEQ ID NO: 4042 | SEQ ID NO: | SEQ ID | SEQ ID: |
| (SEQ ID | 126 | 4023 | | 2562 | NO: 4027 | 130 |
| NO: 4031) | | | | | | |
| 5 | SEQ ID: | SEQ ID: | SEQ ID NO: 4042 | SEQ ID NO: | SEQ ID | SEQ ID: |
| (SEQ ID | 126 | 4024 | | 2562 | NO: 4027 | 130 |
| NO: 4032) | | | | | | |
| 6 | SEQ ID: | SEQ ID: | SEQ ID NO: 4042 | SEQ ID NO: | SEQ ID | SEQ ID: |
| (SEQ ID | 126 | 4025 | | 2562 | NO: 4027 | 130 |
| NO: 4033) | | | | | | |
| 7 | SEQ ID: | SEQ ID: | SEQ ID NO: 4042 | SEQ ID NO: | SEQ ID | SEQ ID: |
| (SEQ ID | 126 | 4026 | | 2562 | NO: 4027 | 130 |
| NO: 4034) | | | | | | |
| 8 | SEQ ID: | SEQ ID: | SEQ ID NO: 4043 | SEQ ID NO: | SEQ ID | SEQ ID: |
| (SEQ ID | 126 | 4021 | | 2562 | NO: 4027 | 130 |
| NO: 4035) | | | | | | |
| 9 | SEQ ID: | SEQ ID: | SEQ ID NO: 4043 | SEQ ID NO: | SEQ ID | SEQ ID: |
| (SEQ ID | 126 | 4022 | | 2562 | NO: 4027 | 130 |
| NO: 4036) | | | | | | |
| 10 | SEQ ID: | SEQ ID: | SEQ ID NO: 4043 | SEQ ID NO: | SEQ ID | SEQ ID: |
| (SEQ ID | 126 | 4014 | | 2562 | NO: 4027 | 130 |
| NO: 4037) | | | | | | |
| 11 | SEQ ID: | SEQ ID: | SEQ ID NO: 4043 | SEQ ID NO: | SEQ ID | SEQ ID: |
| (SEQ ID | 126 | 4023 | | 2562 | NO: 4027 | 130 |
| NO: 4038) | | | | | | |
| 12 | SEQ ID: | SEQ ID: | SEQ ID NO: 4043 | SEQ ID NO: | SEQ ID | SEQ ID: |
| (SEQ ID | 126 | 4024 | | 2562 | NO: 4027 | 130 |
| NO: 4039) | | | | | | |
| 13 | SEQ ID: | SEQ ID: | SEQ ID NO: 4043 | SEQ ID NO: | SEQ ID | SEQ ID: |
| (SEQ ID | 126 | 4025 | | 2562 | NO: 4027 | 130 |
| NO: 4040) | | | | | | |
| 14 | SEQ ID: | SEQ ID: | SEQ ID NO: 4043 | SEQ ID NO: | SEQ ID | SEQ ID: |
| (SEQ ID | 126 | 4026 | | 2562 | NO: 4027 | 130 |
| NO: 4041) | | | | | | |

In certain embodiments, the AAV particle comprises a viral genome which comprises a sequence which has a percent identity to SEQ ID NO: 109. The viral genome may have at least 70%, 75%, 80%, 85%, 90%, 95%, 99% or 100% identity to SEQ ID NO: 9. The viral genome may have 70-80%, 70-90%, 70-99%, 70-100%, 80-85%, 80-90%, 80-95%, 80-99%, 80-100%, 90-95%, 90-99%, or 90-100% identity to SEQ ID NO: 109. As a non-limiting example, the viral genome comprises a sequence which has at least 80% identity to SEQ ID NO: 109. As another non-limiting example, the viral genome comprises a sequence which has at least 85% identity to SEQ ID NO: 109. As another non-limiting example, the viral genome comprises a sequence which has at least 90% identity to SEQ ID NO: 109. As another non-limiting example, the viral genome comprises a sequence which has at least 95% identity to SEQ ID NO: 109. As another non-limiting example, the viral genome comprises a sequence which has at least 99% identity to SEQ ID NO: 109. In some embodiments, the AAV particle comprises the viral genome sequence of SEQ ID NO: 109 differing by no more than 15, 14, 13, 12, 11, 10, 9, 8, 7, 6, 5, 4, 3, 2 or 1 nucleotides from SEQ ID NO: 109. These differing nucleotides may be contiguous, or alternatively, they may be non-contiguous throughout the sequence. In some embodiments, the differing nucleotides are located at the ends of the sequences.

In certain embodiments, the AAV particle comprises a viral genome which comprises a sequence which has a percent identity to SEQ ID NO: 125. The viral genome may have at least 70%, 75%, 80%, 85%, 90%, 95%, 99% or 100% identity to SEQ ID NO: 125. The viral genome may have 70-80%, 70-90%, 70-99%, 70-100%, 80-85%, 80-90%, 80-95%, 80-99%, 80-100%, 90-95%, 90-99%, or 90-100% identity to SEQ ID NO: 125. As a non-limiting example, the viral genome comprises a sequence which has at least 80% identity to SEQ ID NO: 125. As another non-limiting example, the viral genome comprises a sequence which has at least 85% identity to SEQ ID NO: 125. As another non-limiting example, the viral genome comprises a sequence which has at least 90% identity to SEQ ID NO: 125. As another non-limiting example, the viral genome comprises a sequence which has at least 95% identity to SEQ ID NO: 125. As another non-limiting example, the viral genome comprises a sequence which has at least 99% identity to SEQ ID NO: 125. In some embodiments, the AAV particle comprises the viral genome sequence of SEQ ID NO: 125 differing by no more than 15, 14, 13, 12, 11, 10, 9, 8, 7, 6, 5, 4, 3, 2 or 1 nucleotides from SEQ ID NO: 125. These differing nucleotides may be contiguous, or alternatively, they may be non-contiguous throughout the sequence. In some embodiments, the differing nucleotides are located at the ends of the sequences.

In certain embodiments, the AAV particle comprises a viral genome which comprises a nucleotide sequence which has a percent identity to any one of SEQ ID NOs: 4028-4041. The viral genome may have at least 70%, 75%, 80%, 85%, 90%, 95%, 99% or 100% identity to any one of SEQ ID NOs: 4028-4041. The viral genome may have 70-80%, 70-90%, 70-99%, 70-100%, 80-85%, 80-90%, 80-95%, 80-99%, 80-100%, 90-95%, 90-99%, or 90-100% identity to any one of SEQ ID NOs: 4028-4041. As a non-limiting example, the viral genome comprises a nucleotide sequence which has at least 80% identity to any one of SEQ ID NOs: 4028-4041. As another non-limiting example, the viral genome comprises a nucleotide sequence which has at least 85% identity to any one of SEQ ID NOs: 4028-4041. As another non-limiting example, the viral genome comprises a nucleotide sequence which has at least 90% identity to any one of SEQ ID NOs: 4028-4041. As another non-limiting example, the viral genome comprises a nucleotide sequence which has at least 95% identity to any one of SEQ ID NOs: 4028-4041. As another non-limiting example, the viral genome comprises a nucleotide sequence which has at least 99% identity to any one of SEQ ID NOs: 4028-4041. In some embodiments, the AAV particle comprises a viral genome sequence differing by no more than 15, 14, 13, 12, 11, 10, 9, 8, 7, 6, 5, 4, 3, 2 or 1 nucleotides relative to any one of SEQ ID NOs: 4028-4041. These differing nucleotides may be contiguous, or alternatively, they may be non-contiguous throughout the sequence. In some embodiments, the differing nucleotides are located at the ends of the sequences.

In some embodiments, the modulatory polynucleotide sequence of any one of SEQ ID NOs: 4028-4041 is located within (e.g., inserted into) the intron, or replaces a part or all of the intron, of the hβG exon-intron-exon cassette rather than being located 3′ relative to the hβG exon-intron-exon cassette. In some embodiments, one or more of the 5′ ITR, promoter, exon-intron-exon cassette, nucleotide sequence encoding the modulatory polynucleotide, polyA signal sequence, and 3′ITR of the ITR to ITR sequences of any one of SEQ ID NOs: 4028-4041 in Tables 17 and 18 can be replaced with a variant 5′ ITR, promoter, exon-intron-exon cassette, nucleotide sequence encoding the modulatory polynucleotide, polyA signal sequence, and/or 3′ITR sequence, e.g., one or more variant components comprising a nucleotide sequence having at least one, two, or three but no more than four modifications (e.g., substitutions, insertions, and/or deletions) relative to the nucleotide sequence of the non-modified version of the component or components, or a nucleotide sequence with at least 70% (e.g., 80, 85%, 90%, 95%, 96%, 97%, 98%, or 99%) sequence identity to the non-modified version of the component or components.

In some embodiments, the AAV particle comprises a viral genome which comprises, from 5′ to 3′: a 5′ ITR, a promoter (e.g., promoter of CBA, mini-CBA, EF-1, PGK, MP84, SYN, or a variant (e.g., truncated version) of any of the preceding promoters), an exon-intron-exon cassette (e.g., from hβG), a nucleotide sequence encoding a modulatory polynucleotide, wherein the modulatory polynucleotide comprises an siRNA encoding sequence which targets SOD1, a polyA signal sequence (e.g., an rBGpA or hGH poly A signal sequence), and 3′ITR.

In some embodiments, the AAV particle comprises a viral genome which comprises, from 5′ to 3′: a 5′ ITR, a promoter (e.g., promoter of CBA, mini-CBA, EF-1, PGK, MP84, SYN, or a variant (e.g., truncated version) of any of the preceding promoters), an exon-intron-exon cassette (e.g., from hβG), a nucleotide sequence encoding a modulatory polynucleotide comprising an siRNA encoding sequence which targets SOD1 (e.g., hSOD1), wherein the modulatory polynucleotide is inserted into or replaces a portion or all of the intron in the exon-intron-exon cassette, a polyA signal sequence (e.g., an rBGpA or hGH poly A signal sequence), and 3′ITR.

In some embodiments, AAV particle comprises a viral genome which comprises, from 5′ to 3′: a 5′ ITR sequence comprising, e.g., SEQ ID NO: 126, a promoter sequence selected from, e.g., SEQ ID NOs: 4000-4026, an exon-intron-exon cassette selected from e.g., SEQ ID NO: 4023 or 4024, a nucleotide sequence encoding a modulatory polynucleotide comprising an siRNA encoding sequence which targets SOD1 (e.g., hSOD1), a poly A signal sequence selected from, e.g., SEQ ID NO: 129 or 4027, and a 3′ ITR sequence comprising, e.g., SEQ ID NO: 130.

In some embodiments, AAV particle comprises a viral genome which comprises, from 5′ to 3′: a 5′ ITR sequence comprising, e.g., SEQ ID NO: 126, a promoter sequence selected from, e.g., SEQ ID NOs: 4000-4026, an exon-intron-exon cassette selected from e.g., SEQ ID NO: 4023 or 4024, a nucleotide sequence encoding a modulatory polynucleotide comprising an siRNA encoding sequence which targets SOD1 (e.g., hSOD1), wherein the modulatory polynucleotide is inserted into or replaces a portion or all of the intron in the exon-intron-exon cassette, a poly A signal sequence selected from, e.g., SEQ ID NO: 129 or 4027, and a 3′ ITR sequence comprising, e.g., SEQ ID NO: 130.

In some embodiments, the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) an exon, e.g., an exon comprising the nucleotide sequence of any one of SEQ ID NOs: 4045-4048 or variant thereof,
(d) a nucleotide sequence encoding a modulatory polynucleotide (e.g., a SOD1 targeting RNA agent, e.g., a siRNA duplex), e.g., a modulatory polynucleotide comprising the nucleotide sequence of any one of SEQ ID NOs: 2559-2579 and 5022 or variant thereof,
(e) a poly A signal sequence, e.g., a poly A signal sequence comprising the nucleotide sequence of SEQ ID NO: 129 or 4027 or variant thereof, and
(f) a 3′ITR, e.g., a 3′ITR comprising the nucleotide sequence of SEQ ID NO: 126 or 130 or variant thereof,
wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity, relative to the reference sequence, e.g., wherein the reference sequence is a wild-type sequence.

In some embodiments, the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) a first exon, e.g., a first exon comprising the nucleotide sequence of any one of SEQ ID NOs: 4045-4048 or variant thereof,
(d) an intron, e.g., an intron comprising the nucleotide sequence of SEQ ID NO: 4044 or variant thereof,
(e) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
(f) a poly A signal sequence, e.g., a poly A signal sequence comprising the nucleotide sequence of SEQ ID NO: 129 or 4027 or variant thereof, and
(g) a 3′ITR, e.g., a 3′ITR comprising the nucleotide sequence of SEQ ID NO: 126 or 130 or variant thereof,
wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity relative to the reference sequence, e.g., wherein the reference sequence is a wild-type sequence.

In some embodiments, the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) a first exon, e.g., a first exon comprising the nucleotide sequence of any one of SEQ ID NOs: 4045-4048 or variant thereof,
(d) an intron, e.g., an intron comprising the nucleotide sequence of SEQ ID NO: 4044 or variant thereof,
(e) a second exon, e.g., a second exon comprising the nucleotide sequence of any one of SEQ ID NOs: 4045-4048 or variant thereof, optionally wherein the first and second exons are not the same sequence,
(f) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
(g) a poly A signal sequence, e.g., a poly A signal sequence comprising the nucleotide sequence of SEQ ID NO: 129 or 4027 or variant thereof, and
(h) a 3′ITR, e.g., a 3′ITR comprising the nucleotide sequence of SEQ ID NO: 126 or 130 or variant thereof,
wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity relative to the reference sequence, e.g., wherein the reference sequence is a wild-type sequence.

In some embodiments, the viral genome comprises the nucleotide sequence of any one of SEQ ID NOs: 4028-4041, a nucleotide sequence differing by no more than 15, 14, 13, 12, 11, 10, 9, 8, 7, 6, 5, 4, 3, 2 or 1 nucleotides, e.g., substitutions (e.g., conservative substitutions), insertions, or deletions, relative to any one of SEQ ID NOs: 4028-4041, or a nucleotide sequence with at least 70%, 75%, 80%, 85%, 90%, 95%, 99% or 100% identity to any one of SEQ ID NOs: 4028-4041.

In some embodiments, the AAV particle comprises a 5′ITR sequence which comprises at least 15 (e.g., at least 16, 17, 18, 19, 20, 21, 22, or 23) contiguous nucleotides differing by no more than 3, 2 or 1 nucleotides from the 5′ ITR sequence in Tables 16-18. These differing nucleotides may be contiguous, or alternatively, they may be non-contiguous throughout the sequence. In some embodiments, the differing nucleotides are located at the ends of the sequences. The 5′ITR sequence may have 70-80%, 70-90%, 70-99%, 70-100%, 80-85%, 80-90%, 80-95%, 80-99%, 80-100%, 90-95%, 90-99%, or 90-100% identity to the 5′ ITR sequence in Table 16.

In some embodiments, the AAV particle comprises a Albumin derived filler sequence which comprises at least 15 (e.g., at least 16, 17, 18, 19, 20, 21, 22, or 23) contiguous nucleotides differing by no more than 3, 2 or 1 nucleotides from the Albumin derived filler sequence in Table 16. These differing nucleotides may be contiguous, or alternatively, they may be non-contiguous throughout the sequence. In some embodiments, the differing nucleotides are located at the ends of the sequences. The Albumin derived filler sequence may have 70-80%, 70-90%, 70-99%, 70-100%, 80-85%, 80-90%, 80-95%, 80-99%, 80-100%, 90-95%, 90-99%, or 90-100% identity to the Albumin derived filler sequence in Table 16.

In some embodiments, the AAV particle comprises a promoter sequence (e.g., an H1 promoter sequence or a promoter sequence in Table 17, 18 or 7) which comprises at least 15 (e.g., at least 16, 17, 18, 19, 20, 21, 22, or 23) contiguous nucleotides differing by no more than 7, 6, 5, 4, 3, 2 or 1 nucleotides from the H1 promoter sequence in Table 16 or a promoter sequence in Table 17, 18, or 7. These differing nucleotides may be contiguous, or alternatively, they may be non-contiguous throughout the sequence. In some embodiments, the differing nucleotides are located at the ends of the sequences. The promoter sequence (e.g., an H1 promoter sequence or a promoter sequence in Tables 17, 18 or 6) may have 70-80%, 70-90%, 70-99%, 70-100%, 80-85%, 80-90%, 80-95%, 80-99%, 80-100%, 90-95%, 90-99%, or 90-100% identity to the H1 promoter sequence in Table 16 or a promoter sequence in Tables 17, 18 or 7.

In some embodiments, the AAV particle comprises a first and/or second exon, an intron, or a first exon, intron, and a second exon (“exon-intron-exon cassette”) (e.g., a first and/or second exon, intron, or exon-intron-exon cassette from Tables 17, 18 or 7, e.g., hβG first and/or second exon, intron, or exon-intron-exon cassette) which comprises at least 15 (e.g., at least 16, 17, 18, 19, 20, 21, 22, or 23) contiguous nucleotides differing by no more than 7, 6, 5, 4, 3, 2 or 1 nucleotides from the first and/or second exon, intron, or exon-intron-exon cassette in Tables 17, 18 or 9. These differing nucleotides may be contiguous, or alternatively, they may be non-contiguous throughout the sequence. In some embodiments, the differing nucleotides are located at the ends of the sequences. The first and/or second exon, intron, or exon-intron-exon cassette (e.g., a first and/or second exon, intron, or exon-intron-exon cassette from Tables 17, 18 or 9, e.g., hβG first and/or second exon, intron or exon-intron-exon cassette) may have 70-80%, 70-90%, 70-99%, 70-100%, 80-85%, 80-90%, 80-95%, 80-99%, 80-100%, 90-95%, 90-99%, or 90-100% identity to the first and/or second exon, intron, or exon-intron-exon cassette in Tables 17, 18 or 9.

In some embodiments, the AAV particle comprises a Poly A sequence which comprises at least 15 (e.g., at least 16, 17, 18, 19, 20, 21, 22, or 23) contiguous nucleotides differing by no more than 3, 2 or 1 nucleotides from the polyA sequence in Tables 16-18 or 8. These differing nucleotides may be contiguous, or alternatively, they may be non-contiguous throughout the sequence. In some embodiments, the differing nucleotides are located at the ends of the sequences. The Poly A sequence may have 70-80%, 70-90%, 70-99%, 70-100%, 80-85%, 80-90%, 80-95%, 80-99%, 80-100%, 90-95%, 90-99%, or 90-100% identity to the polyA sequence in Tables 16-18 or 8.

In some embodiments, the AAV particle comprises a 3′ITR sequence which comprises at least 15 (e.g., at least 16, 17, 18, 19, 20, 21, 22, or 23) contiguous nucleotides differing by no more than 3, 2 or 1 nucleotides from the 3′ ITR sequence in Tables 16-18. These differing nucleotides may be contiguous, or alternatively, they may be non-contiguous throughout the sequence. In some embodiments, the differing nucleotides are located at the ends of the sequences. The 3′ITR sequence may have 70-80%, 70-90%, 70-99%, 70-100%, 80-85%, 80-90%, 80-95%, 80-99%, 80-100%, 90-95%, 90-99%, or 90-100% identity to the 3′ITR in Tables 16-18.

AAV particles may be modified to enhance the efficiency of delivery. Such modified AAV particles comprising the nucleic acid sequence encoding the siRNA molecules of the present disclosure can be packaged efficiently and can be used to successfully infect the target cells at high frequency and with minimal toxicity.

In some embodiments, the AAV particle comprising a nucleic acid sequence encoding the siRNA molecules of the present disclosure may be a human serotype AAV particle. Such human AAV particle may be derived from any known serotype, e.g., from any one of serotypes AAV1-AAV11. As non-limiting examples, AAV particles may be vectors comprising an AAV1-derived genome in an AAV1-derived capsid; vectors comprising an AAV2-derived genome in an AAV2-derived capsid; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV particle comprising a nucleic acid sequence for encoding siRNA molecules of the present disclosure may be a pseudotyped hybrid or chimeric AAV particle which contains sequences and/or components originating from at least two different AAV serotypes. Pseudotyped AAV particles may be vectors comprising an AAV genome derived from one AAV serotype and a capsid protein derived at least in part from a different AAV serotype. As non-limiting examples, such pseudotyped AAV particles may be vectors comprising an AAV2-derived genome in an AAV1-derived capsid; or vectors comprising an AAV2-derived genome in an AAV6-derived capsid; or vectors comprising an AAV2-derived genome in an AAV4-derived capsid; or an AAV2-derived genome in an AAV9-derived capsid. In like fashion, the present disclosure contemplates any hybrid or chimeric AAV particle.

In other embodiments, AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety).

In some aspects, the AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In other embodiments, the siRNA molecules of the present disclosure can be encoded in plasmid vectors, viral vectors (e.g., AAV vectors), genome or other nucleic acid expression vectors for delivery to a cell.

DNA expression plasmids can be used to stably express the siRNA duplexes or dsRNA of the present disclosure in cells and achieve long-term inhibition of target gene.

In one aspect, the sense and antisense strands of a siRNA duplex encoded by a SOD1 targeting polynucleotide are typically linked by a short spacer sequence leading to the expression of a stem-loop structure termed short hairpin RNA (shRNA). The hairpin is recognized and cleaved by Dicer, thus generating mature siRNA molecules.

According to the present disclosure, AAV particles comprising the nucleic acids of the siRNA molecules targeting SOD1 mRNA can further comprise, a capsid protein chosen from VOY101, AAVPHP.B (PHP.B), AAVPHP.N (PHP.N), AAV1, AAV2, AAV5, AAV9, AAV9 K449R, AAVrh10, and variants thereof.

In some embodiments, the siRNA duplexes or dsRNA of the present disclosure when expressed suppress (or degrade) target mRNA (i.e. SOD1). Accordingly, the siRNA duplexes or dsRNA encoded by a SOD1 targeting polynucleotide can be used to substantially inhibit SOD1 gene expression in a cell, for example a motor neuron. In some aspects, the inhibition of SOD1 gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The SOD1 gene can be either a wild type gene or a mutated SOD1 gene with at least one mutation. Accordingly, the protein is either wild type protein or a mutated polypeptide with at least one mutation.

### III. Viral Production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid comprising a transgene encoding a polynucleotide (e.g., a SOD1 targeting siRNA described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in a capsid protein, e.g., a capsid protein described herein (e.g., a capsid protein listed in Table 2, e.g., a VOY101 capsid protein or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In various embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. The non-structural protein may include any of the native or wild type Rep78, Rep68, Rep52, and/or Rep40 proteins or a chimeric protein thereof.

In certain embodiments, contacting occurs via transient transfection, viral transduction, and/or electroporation.

In certain embodiments, the viral production cell is selected from a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include, in various embodiments, at least one inverted terminal repeat (ITR) and may include mammalian DNA.

Also provided are AAV particles and viral vectors produced according to the methods described herein.

In various embodiments, the AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In certain embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In certain embodiments, the AAV particles are produced in a mammalian cell (e.g., HEK293 cell) using a method described herein. As a non-limiting example, the mammalian cell is contacted using viral transduction which may include multiplasmid transient transfection (such as triple plasmid transient transfection).

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV viral genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in their entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Δie-1 promoter, has about 20% of the transcriptional activity of that ie-1 promoter. A promoter substantially homologous to the Δie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in their entirety as related to the 293-10-3 packaging cell line and uses thereof.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In certain embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900II™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV viral genomes encoding SOD1 targeting siRNA described herein may be useful in the fields of human disease, veterinary applications and a variety of in vivo and in vitro settings. The AAV particles of the present disclosure may be useful in the field of medicine for the treatment, prophylaxis, palliation, or amelioration of neurological or neuromuscular diseases and/or disorders. In some embodiments, the AAV particles of the disclosure are used for the prevention and/or treatment of SOD1-related disorders.

Various embodiments of the disclosure herein provide a pharmaceutical composition comprising the AAV particle described herein and a pharmaceutically acceptable excipient.

Various embodiments of the disclosure herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of: intravenous, intracerebroventricular, intraparenchymal, intrathecal, subpial, and intramuscular, or a combination thereof. Certain embodiments of the method provide that the subject is treated for SOD1-related disorders. In one aspect of the method, a pathological feature of the SOD1-related disorder is alleviated and/or the progression of the SOD1-related disorder is halted, slowed, ameliorated, or reversed.

Various embodiments of the disclosure herein describe a method of increasing the level of SOD1 targeting siRNAs in the central nervous system of a subject in need thereof comprising administering to said subject via infusion, an effective amount of the pharmaceutical composition described herein.

Also described herein are compositions, methods, processes, kits and devices for the design, preparation, manufacture and/or formulation of AAV particles. In some embodiments, payloads, such as but not limited to payloads comprising SOD1 targeting siRNAs, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of SOD1-related disorders. Such methods may involve gene replacement or gene activation. Such outcomes are achieved by utilizing the methods and compositions taught herein.

### IV. Introduction into Cells

To ensure the chemical and biological stability of siRNA duplexes, it is important to deliver polynucleotides encoding the siRNAs inside the target cells. The polynucleotides of the present disclosure may be introduced into cells using any of a variety of approaches.

In some embodiments, the polynucleotide of the present disclosure is introduced into a cell by contacting the cell with the polynucleotide. In some embodiments, the polynucleotide is introduced into a cell by contacting the cell with a composition comprising the polynucleotide and a lipophilic carrier. In other embodiments, the polynucleotide is introduced into a cell by transfecting or infecting the cell with a vector comprising nucleic acid sequences capable of producing the siRNA duplex when transcribed in the cell.

In some embodiments, the siRNA duplex is introduced into a cell by injecting into the cell a vector comprising nucleic acid sequences capable of producing the siRNA duplex when transcribed in the cell.

In other embodiments, the polynucleotides of the present disclosure may be delivered into cells by electroporation (e.g. U.S. Patent Publication No. 20050014264; the content of which is herein incorporated by reference in its entirety).

In addition, the siRNA molecules inserted into viral vectors (e.g. AAV vectors) may be delivered into cells by viral infection. These viral vectors are engineered and optimized to facilitate the entry of siRNA molecule into cells that are not readily amendable to transfection. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In some embodiments, the cells may include, but are not limited to, cells of mammalian origin, cells of human origins, embryonic stem cells, induced pluripotent stem cells, neural stem cells, and neural progenitor cells.

### V. Pharmaceutical Compositions and Formulation

The present disclosure additionally provides a method for treating SOD1-related disorders and disorders related to deficiencies in the function or expression of SOD1 in a mammalian subject, including a human subject, comprising administering to the subject any of the AAV polynucleotides or AAV genomes described herein (i.e., “vector genomes,” “viral genomes,” or “VGs”) or administering to the subject a particle comprising said AAV polynucleotide or AAV genome, or administering to the subject any of the described compositions, including pharmaceutical compositions.

As used herein the term “composition” comprises an AAV polynucleotide or AAV genome or AAV particle and at least one excipient.

As used herein the term “pharmaceutical composition” comprises an AAV polynucleotide or AAV genome or AAV particle and one or more pharmaceutically acceptable excipients.

Although the descriptions of pharmaceutical compositions, e.g., AAV comprising a payload encoding a SOD1 targeting siRNA to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is a SOD1 targeting polynucleotide (e.g., a SOD1 targeting RNAi, e.g., a siRNA duplex for inhibiting expression of SOD1) described herein, or a variant thereof.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### VI. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

In some embodiments, the formulations described herein may contain at least one SOD1 targeting polynucleotide (e.g., a SOD1 targeting RNAi, e.g., a siRNA duplex for inhibiting expression of SOD1). As a non-limiting example, the formulations may contain 1, 2, 3, 4 or 5 polynucleotide that target the SOD1 gene at different sites.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding the SOD1 targeting polynucleotides may be formulated to optimize baricity and/or osmolality. In some embodiments, the baricity and/or osmolality of the formulation may be optimized to ensure optimal drug distribution in the central nervous system or a region or component of the central nervous system.

In some embodiments, the AAV particles of the disclosure may be formulated in PBS with 0.001% of pluronic acid (F-68) at a pH of about 7.0.

In some embodiments, the AAV particles of the disclosure may be formulated in PBS, in combination with an ethylene oxide/propylene oxide copolymer (also known as pluronic or poloxamer).

In some embodiments, the AAV particles of the disclosure may be formulated in PBS with 0.001% pluronic acid (F-68) (poloxamer 188) at a pH of about 7.0.

In some embodiments, the AAV particles of the disclosure may be formulated in PBS with 0.001% pluronic acid (F-68) (poloxamer 188) at a pH of about 7.3.

In some embodiments, the AAV particles of the disclosure may be formulated in PBS with 0.001% pluronic acid (F-68) (poloxamer 188) at a pH of about 7.4.

In some embodiments, the AAV particles of the disclosure may be formulated in a solution comprising sodium chloride, sodium phosphate and an ethylene oxide/propylene oxide copolymer.

In some embodiments, the AAV particles of the disclosure may be formulated in a solution comprising sodium chloride, sodium phosphate dibasic, potassium chloride, potassium phosphate monobasic, and poloxamer 188/pluronic acid (F-68).

In some embodiments, the AAV particles of the disclosure may be formulated in a solution comprising 192 mM sodium chloride, 10 mM sodium phosphate (dibasic), 2.7 mM potassium chloride, 2 mM potassium phosphate (monobasic) and 0.001% pluronic F-68 (v/v), at pH 7.4. This formulation is referred to as Formulation 1 in the present disclosure.

In some embodiments, the AAV particles of the disclosure may be formulated in a solution comprising about 192 mM sodium chloride, about 10 mM sodium phosphate dibasic and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate dibasic in the final solution may be 1 mM-50 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

### Excipients

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### VII. Uses and Applications

Provided in the present disclosure are methods for introducing the SOD1 targeting polynucleotides described herein into cells, the method comprising introducing into said cells any of the polynucleotides in an amount sufficient for degradation of target SOD1 mRNA to occur. In some aspects, the cells may be stem cells, neurons such as motor neurons, muscle cells and glial cells such as astrocytes.

Described here are methods for delivering AAV particles to the spinal cord, for the treatment of disorders associated with the spinal cord, such as, but not limited to motor neuron disease (e.g., ALS). In certain embodiments, these methods result in trans-synaptic transmission.

Disclosed herein are also methods for treating ALS associated with abnormal SOD1 function in a subject in need of treatment. The method optionally comprises administering to the subject a therapeutically effective amount of a composition comprising or encoding at least one siRNA duplex targeting SOD1 gene. Said siRNA duplex will silence SOD1 gene expression and inhibit SOD1 protein production and reduce one or more symptoms of ALS in the subject such that ALS is therapeutically treated.

In some embodiment, administration to a subject will reduce the expression of mutant and/or wild-type SOD1 in the central nervous system (e.g., brain tissue, spinal cord, and/or motor neurons) and/or and the reduction of expression of the mutant and/or wild-type SOD1 will reduce the effects of ALS in a subject.

In some embodiment, the administration is to a subject who is in the early stages of ALS. Early stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the AAV viral particle may reduce the severity and/or occurrence of the symptoms of ALS.

In some embodiment, the administration is to a subject who is in the middle stages of ALS. The middle stage of ALS includes, but is not limited to, more widespread muscle symptoms as compared to the early stage, some muscles are paralyzed while others are weakened or unaffected, continued muscle twitchings (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the AAV particles may reduce the severity and/or occurrence of the symptoms of ALS.

In one embodiment, the administration is to a subject who is in the late stages of ALS. The late stage of ALS includes, but is not limited to, voluntary muscles which are mostly paralyzed, the muscles that help move air in and out of the lungs are severely compromised, mobility is extremely limited, poor respiration may cause fatigue, fuzzy thinking, headaches and susceptibility to infection or diseases (e.g., pneumonia), speech is difficult and eating or drinking by mouth may not be possible.

In some embodiments, the AAV particles may be used to treat a subject with ALS who has a C9orf72 mutation, TDP-43 mutations, and/or FUS mutations.

In some embodiments, the SOD1 targeting polynucleotide of the present disclosure or the composition comprising or encoding the polynucleotides is administered to the central nervous system of the subject. In other embodiments, the siRNA duplex of the present disclosure or the composition comprising it is administered to the muscles of the subject

In particular, the SOD1 targeting polynucleotides may be delivered into specific types of targeted cells, including motor neurons; glial cells including oligodendrocyte, astrocyte and microglia; and/or other cells surrounding neurons such as T cells. Studies in human ALS patients and animal SOD1 ALS model implicated that glial cells play an early role in the dysfunction and death of ALS neurons. Normal SOD1 in the surrounding, protective glial cells can prevent the motor neurons from dying even though mutant SOD1 is present in motor neurons (e.g., reviewed by Philips and Rothstein, Exp. Neurol., 2014, May 22. pii: 50014-4886(14)00157-5; the content of which is incorporated herein by reference in its entirety).

In some specific embodiments, at least one siRNA duplex targeting SOD1 gene used as a therapy for ALS is inserted in an AAV viral genome.

In some embodiments, the present composition is administered as a single therapeutic or combination therapeutics for the treatment of ALS.

The viral vectors comprising or encoding siRNA duplexes targeting SOD1 gene may be used in combination with one or more other therapeutic, agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the SOD1 targeting polynucleotides of the present disclosure can be small molecule compounds which are antioxidants, anti-inflammatory agents, anti-apoptosis agents, calcium regulators, antiglutamatergic agents, structural protein inhibitors, and compounds involved in metal ion regulation.

Compounds used in combination for treating ALS may include, but are not limited to, agents that reduce oxidative stress, such as free-radical scavengers, or Radicava (edaravone), antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8(4)); Beat-lactam antibiotics; Pramipexole (a dopamine agonist) (Wang et al., Amyotrophic Lateral Scler., 2008, 9(1), 50-58); Nimesulide in U.S. Patent Publication No. 20060074991; Diazoxide disclosed in U.S. Patent Publication No. 20130143873); pyrazolone derivatives disclosed in US Patent Publication No. 20080161378; free radical scavengers that inhibit oxidative stress-induced cell death, such as bromocriptine (US. Patent Publication No. 20110105517); phenyl carbamate compounds discussed in PCT Patent Publication No. 2013100571; neuroprotective compounds disclosed in U.S. Pat. Nos. 6,933,310 and 8,399,514 and US Patent Publication Nos. 20110237907 and 20140038927; and glycopeptides taught in U.S. Patent Publication No. 20070185012; the content of each of which is incorporated herein by reference in their entirety.

Therapeutic agents that may be used in combination therapy with the siRNA duplexes targeting SOD1 gene of the present disclosure may be hormones or variants that can protect neuron loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

Neurotrophic factors may be used in combination therapy with the siRNA duplexes targeting SOD1 gene of the present disclosure for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle comprising at least one siRNA duplex targeting SOD1 gene may be co-administered with AAV particle expressing neurotrophic factors such as AAV-IGF-I (Vincent et al., Neuromolecular medicine, 2004, 6, 79-85; the content of which is incorporated herein by reference in its entirety) and AAV-GDNF (Wang et al., J Neurosci., 2002, 22, 6920-6928; the content of which is incorporated herein by reference in its entirety).

### VIII. Dosing and Administration

### Administration

In some aspects, the present disclosure provides administration and/or delivery methods for vectors and viral particles, e.g., AAV particles, a SOD1 targeting polynucleotide or a variant thereof, for the prevention, treatment, or amelioration of diseases or disorders of the CNS. For example, administration of the AAV particles prevents, treats, or ameliorates SOD1-related disorders. Thus, robust widespread SOD1 targeting siRNA distribution throughout the CNS and periphery is desired for maximal efficacy. Particular target tissues for administration or delivery include CNS tissues, more specifically, brain tissue.

In some aspects, the present disclosure relates to a method of delivering an exogenous siRNA duplex for inhibiting expression of SOD1 to a subject, comprising administering an effective amount of the pharmaceutical composition or the AAV particle described herein to a subject, thereby delivering the exogenous siRNA duplex to the subject.

In specific embodiments, compositions including AAV particles comprising at least one SOD1 targeting polynucleotide may be administered in a way which allows them to enter the central nervous system and penetrate into motor neurons.

The AAV particles of the present disclosure may be administered by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), intracranial (into the skull), picutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraparenchymal (into the substance of), intrathecal (into the spinal canal), intraperitoneal, (infusion or injection into the peritoneum), intravesicular infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, subpial, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, AAV particles of the present disclosure are administered so as to be delivered to a target cell or tissue. Delivery to a target cell results in SOD1 targeting siRNA expression. A target cell may be any cell in which it is considered desirable to increase SOD1 targeting siRNA expression levels. A target cell may be a CNS cell. Non-limiting examples of such cells and/or tissues include, dorsal root ganglia and dorsal columns, proprioceptive sensory neurons, Clark's column, gracile and cuneate nuclei, cerebellar dentate nucleus, corticospinal tracts and the cells comprising the same, Betz cells, and cells of the heart.

In some embodiments, compositions may be administered in a way that allows them to cross the blood-brain barrier, vascular barrier, or other epithelial barrier.

In some embodiments, delivery of SOD1 targeting siRNA by adeno-associated virus (AAV) particles to cells of the central nervous system (e.g., parenchyma) comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In some embodiments, the AAV particles may be delivered by systemic delivery. In some embodiments, the systemic delivery may be by intravascular administration. In some embodiments, the systemic delivery may be by intravenous (IV) administration.

In some embodiments, the AAV particles may be delivered by intravenous delivery.

In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, https://doi.org/10.1038/s41573-021-00139-y (2021)), Burgess et al. (Expert Rev Neurother. 15(5): 477-491 (2015)), and/or Hsu et al. (PLOS One 8(2): 1-8), the contents of which are incorporated herein by reference in its entirety.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by thalamic delivery.

In some embodiments, the AAV particles may be delivered by intracerebral delivery.

In some embodiments, the AAV particles may be delivered by intracardiac delivery.

In some embodiments, the AAV particles may be delivered by intracranial delivery. (See, e.g., U.S. Pat. No. 8,119,611; the content of which is incorporated herein by reference in its entirety).

In some embodiments, the AAV particles may be delivered by intra cisterna magna (ICM) delivery (i.e., the space around and below the cerebellum via the opening between the skull and the top of the spine). In certain embodiments, the pharmaceutical compositions of the present disclosure may be administered to the cisterna magna in a therapeutically effective amount to transduce spinal cord motor neurons and/or astrocytes. In some embodiments, the AAV particles may be delivered via infusion or injection to the cisterna magna. The administration to the cisterna magna may occur via a bolus injection. Depending on the severity of the symptoms and the responsiveness of the subject to the therapy, the bolus injection may be administered once per week, once per month, once every two months, once every three months, once every 6 months or annually. The administration to the cisterna magna may occur via a catheter or canula. In other embodiments, the administration is achieved by use of an infusion pump. The administration may occur continually over a period of at least several days.

In some embodiments, the AAV particles may be delivered by direct (intraparenchymal) injection into an organ (e.g., CNS (brain or spinal cord)). In some embodiments, the intraparenchymal delivery may be to any region of the brain or CNS. In some embodiments, the AAV particles may be administered by intraparenchymal injection to the CNS, the brain and/or the spinal cord. As a non-limiting example, the AAV particles of the present disclosure may be administered to a subject by intraparenchymal injection. In certain embodiments, the intraparenchymal injection may be a spinal intraparenchymal injection, wherein the pharmaceutical compositions may be administered directly to the tissue of the spinal cord. In certain embodiments, the intraparenchymal injection may be a CNS (central nervous system) intraparenchymal injection wherein the pharmaceutical compositions may be administered directly to the tissue of the CNS.

In some embodiments, the AAV particles may be administered by intraparenchymal injection and intrathecal injection. In certain embodiments, the pharmaceutical compositions of the present disclosure may be administered by intraparenchymal injection and intrastriatal injection.

In some embodiments, the AAV particles may be delivered by intrastriatal injection.

In some embodiments, the AAV particles may be delivered into the putamen.

In some embodiments, the AAV particles may be delivered into the spinal cord.

In some embodiments, the AAV particles of the present disclosure may be administered to the ventricles of the brain.

In some embodiments, the AAV particles of the present disclosure may be administered to the ventricles of the brain by intracerebroventricular delivery.

In some embodiments, the AAV particles of the present disclosure may be administered by intramuscular delivery. Rizvanov et al. demonstrated for the first time that siRNA molecules, targeting mutant human SOD1 mRNA, is taken up by the sciatic nerve, retrogradely transported to the perikarya of motor neurons, and inhibits mutant SOD1 mRNA in SOD1^{G93A} transgenic ALS mice (Rizvanov A A et al., Exp. Brain Res., 2009, 195(1), 1-4; the content of which is incorporated herein by reference in its entirety). Another study also demonstrated that muscle delivery of AAV expressing small hairpin RNAs (shRNAs) against the mutant SOD1 gene, led to significant mutant SOD1 knockdown in the muscle as well as innervating motor neurons (Towne C et al., Mol Ther., 2011; 19(2): 274-283; the content of which is incorporated herein by reference in its entirety).

In some embodiments, AAV particles that express siRNA duplexes of the present disclosure may be administered to a subject by peripheral injections and/or intranasal delivery. It was disclosed in the art that the peripheral administration of AAV particles for siRNA duplexes can be transported to the central nervous system, for example, to the motor neurons (e.g., U.S. Patent Publication Nos. 20100240739; and 20100130594; the content of each of which is incorporated herein by reference in their entirety).

In some embodiments, the AAV particles of the present disclosure are administered by more than one route described above. As a non-limiting example, the AAV particles may be administered by intravenous delivery and thalamic delivery.

In some embodiments, the AAV particles of the present disclosure are administered by more than one route described above. As a non-limiting example, the AAV particles may be administered by intravenous delivery and intracerebral delivery.

In some embodiments, the AAV particles of the present disclosure are administered by more than one route described above. As a non-limiting example, the AAV particles may be administered by intravenous delivery and intracranial delivery.

In some embodiments, the AAV particles of the present disclosure are administered by more than one route described above. In some embodiments, the AAV particles of the present disclosure may be delivered by intrathecal and intracerebroventricular administration.

In some embodiments, the AAV particles may be delivered to a subject to preserve neurons. The neurons may be primary and/or secondary sensory neurons. In some embodiments, AAV particles are delivered to dorsal root ganglia and/or neurons thereof. In some embodiments, AAV particles are delivered to motor neurons.

In some embodiments, the AAV particles may be delivered to a subject to improve and/or correct mitochondrial dysfunction.

In some embodiments, administration of the AAV particles may preserve and/or correct function in the sensory pathways.

In some embodiments, the AAV particles may be delivered via intravenous (IV), intracerebroventricular (ICV), intraparenchymal, and/or intrathecal (IT) infusion and the therapeutic agent may also be delivered to a subject via intramuscular (IM) limb infusion in order to deliver the therapeutic agent to the skeletal muscle. Delivery of AAVs by intravascular limb infusion is described by Gruntman and Flotte, Human Gene Therapy Clinical Development, 2015, 26(3), 159-164, the contents of which are herein incorporated by reference in their entirety.

In some embodiments, delivery of viral vector pharmaceutical compositions in accordance with the present disclosure to cells of the central nervous system (e.g., parenchyma) comprises a rate of delivery defined by VG/hour=mL/hour*VG/mL, wherein VG is viral genomes, VG/mL is composition concentration, and mL/hour is rate of infusion.

In some embodiments, delivery of AAV particle pharmaceutical compositions in accordance with the present disclosure to cells of the central nervous system (e.g., parenchyma) comprises infusion of up to 1 mL. In some embodiments, delivery of viral vector pharmaceutical compositions in accordance with the present disclosure to cells of the central nervous system (e.g., parenchyma) may comprise infusions of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL.

In certain embodiments, the AAV particle described herein is administered via intraparenchymal (IPa) infusion at any level of the spinal cord, at a single or at multiple sites, at a volume of more than 1 uL. In certain embodiments, a volume of 1 uL-100 uL is administered.

The spinal cord is situated within the spine. The spine consists of a series of vertebral segments. There are 7 cervical (C1-C7), 12 thoracic (T1-T12), 5 lumbar (L1-L5), and 5 sacral (S1-S5) vertebral segments. Intraparenchymal injection or infusion into the spinal cord of AAV particles described herein may occur at one or multiple of these vertebral segments. For example, intraparenchymal injection or infusion into the spinal cord of AAV particles described herein may occur at 1, 2, 3, 4, 5, or more than 5 sites. The intraparenchymal injection or infusion sites may be at one or more regions independently selected from the cervical spinal cord, the thoracic spinal cord, the lumbar spinal cord, and the sacral spinal cord. In some embodiments, AAV particles described herein are administered via intraparenchymal (IPa) infusion at two sites into the spinal cord.

In some embodiments, the AAV particle described herein may be administered via intraparenchymal (IPa) infusion to one or more sites (e.g., 2, 3, 4 or 5 sites) selected from C1, C2, C3, C4, C5, C6, and C7. In some embodiments, the AAV particle described herein may be administered via intraparenchymal (IPa) infusion to one or more sites (e.g., 2, 3, 4 or 5 sites) selected from T1, T2, T3, T4, T5, T6, T7, T8, T9, T10, T11, and T12. In some embodiments, the AAV particle described herein may be administered via intraparenchymal (IPa) infusion to one or more sites (e.g., 2, 3, 4 or 5 sites) selected from L1, L2, L3, L4, and L5. In some embodiments, the AAV particle described herein may be administered via intraparenchymal (IPa) infusion to one or more sites (e.g., 2, 3, 4 or 5 sites) selected from S1, S2, S3, S4, and S5.

In some embodiments, the AAV particle described herein may be administered via intraparenchymal (IPa) infusion at one or more sites (e.g., 2, 3, 4 or 5 sites) selected from C1, C2, C3, C4, C5, C6, C7, T1, T2, T3, T4, T5, T6, T7, T8, T9, T10, T11, T12, L1, L2, L3, L4, L5, S1, S2, S3, S4, and S5. As a non-limiting example, the administration is at two sites and may include one site from the cervical spinal cord region (e.g., C1-C7) and one site from the thoracic spinal cord region (e.g., T1-T12). As a non-limiting example, the administration is at two sites and may include one site from the thoracic spinal cord region (e.g., T1-T12) and one site from the lumbar spinal cord region (e.g., L1-L5).

In certain embodiments, the AAV particle described herein encoding siRNA molecules may be administered via intraparenchymal (IPa) infusion at two sites. The AAV particles may be delivered at the same or different volume for both sites. The AAV particles may be delivered at the same or different volumes for both sites. The AAV particles may be delivered at the same or different infusion rates for both sites.

In some embodiments, IPa infusions (e.g., spinal cord) may result in delivery of the pharmaceutical compositions (i.e., AAV particles) along the extent of the rostral-caudal axis of the spinal cord. In some embodiments, IPa infusions (e.g., spinal cord) yield a rostrocaudal gradient of AAV particle transmission. In some embodiments, IPa infusions (e.g., spinal cord) result in delivery of the pharmaceutical compositions to regions distal to the injection site. While not wishing to be bound by theory, AAV particles of the disclosure may travel the length of the rostral caudal axis of the spinal cord subsequent to IPa infusion at a particular site. In other words, the AAV particles may not confined to the immediate vicinity of the injection site. As a non-limiting example, the AAV particles may be transported by a trans-synaptic (across the synapse) mechanism. This trans-synaptic mechanism may follow a tract or channel present along the rostral-caudal axis of the spinal cord. Additional methods relating to IPa administration are known in the art, including for example U.S. Patent Application Publication US20210254103, which is hereby incorporated by reference in its entirety. Details regarding the effect of SOD1 siRNAs in vitro and in vivo, as well as intraparenchymal administration of SOD1 siRNAs packaged in AAVs, are described in PCT Patent Application Publication WO2020010042, which is hereby incorporated by reference in its entirety, see, e.g., pp 154-175.

In some embodiments, delivery of AAV particle pharmaceutical compositions in accordance with the present disclosure to cells of the central nervous system (e.g., parenchyma) comprises infusion of between about 1 mL to about 120 mL. In some embodiments, delivery of viral vector pharmaceutical compositions in accordance with the present disclosure to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In some embodiments delivery of AAV particles to cells of the central nervous system (e.g., parenchyma) comprises infusion of at least 3 mL. In some embodiments, delivery of AAV particles to cells of the central nervous system (e.g., parenchyma) consists of infusion of 3 mL. In some embodiments, delivery of AAV particles to cells of the central nervous system (e.g., parenchyma) comprises infusion of at least 10 mL. In some embodiments, delivery of AAV particles to cells of the central nervous system (e.g., parenchyma) consists of infusion of 10 mL.

In some embodiments, the volume of the AAV particle pharmaceutical composition delivered to the cells of the central nervous system (e.g., parenchyma) of a subject is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In some embodiments, the volume of the AAV particle pharmaceutical composition delivered to a region in both hemispheres of a subject brain is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In certain embodiments, AAV particle or viral vector pharmaceutical compositions in accordance with the present disclosure may be administered at about 10 to about 600 μl/site, about 50 to about 500 μl/site, about 100 to about 400 μl/site, about 120 to about 300 μl/site, about 140 to about 200 μl/site, or about 160 μl/site.

In some embodiments, the total volume delivered to a subject may be split between one or more administration sites e.g., 1, 2, 3, 4, 5, or more than 5 sites. In some embodiments, the total volume is split between administration to the left and right hemisphere.

### Delivery of AAV Particles

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particles of the present disclosure may be administered or delivered using the methods for the delivery of AAV virions described in European Patent Application No. EP1857552, the contents of which are herein incorporated by reference in their entirety.

In some embodiments, the AAV particle or pharmaceutical compositions of the present disclosure may be administered or delivered using the methods for delivering proteins using AAV particles described in European Patent Application No. EP2678433, the contents of which are herein incorporated by reference in their entirety.

In some embodiments, the viral vector encoding a SOD1 targeting siRNA may be administered or delivered using the methods for delivering DNA molecules using AAV particles described in U.S. Pat. No. 5,858,351, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the viral vector encoding a SOD1 targeting siRNA may be administered or delivered using the methods for delivering AAV virions described in U.S. Pat. No. 6,325,998, the contents of which are herein incorporated by reference in their entirety.

In some embodiments, the viral vector encoding a SOD1 targeting siRNA may be administered or delivered using the methods for delivering DNA to muscle cells described in U.S. Pat. No. 6,335,011, the contents of which are herein incorporated by reference in their entirety.

In some embodiments, the viral vector encoding a SOD1 targeting siRNA may be administered or delivered using the methods for delivering DNA to muscle cells and tissues described in U.S. Pat. No. 6,610,290, the contents of which are herein incorporated by reference in their entirety.

In some embodiments, the viral vector encoding a SOD1 targeting siRNA may be administered or delivered using the methods for delivering DNA to muscle cells described in U.S. Pat. No. 7,704,492, the contents of which are herein incorporated by reference in their entirety.

In some embodiments, the viral vector encoding a SOD1 targeting siRNA may be administered or delivered using the methods for delivering a payload to skeletal muscles described in U.S. Pat. No. 7,112,321, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload for the treatment of Alzheimer disease described in U.S. Pat. No. 8,318,687, the contents of which are herein incorporated by reference in their entirety.

In some embodiments, the AAV particle or pharmaceutical compositions of the present disclosure may be administered or delivered using the methods for delivering a payload described in International Patent Publication No. WO2012144446, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

In some embodiments, the AAV particle or pharmaceutical compositions of the present disclosure may be administered or delivered using the methods for delivering a payload described in International Patent Publication No. WO2001096587, the contents of which are herein incorporated by reference in their entirety.

In some embodiments, the AAV particle or pharmaceutical compositions of the present disclosure may be administered or delivered using the methods for delivering a payload to muscle tissue described in International Patent Publication No. WO2002014487, the contents of which are herein incorporated by reference in their entirety.

In some embodiments, a catheter may be used to administer the AAV particles. In certain embodiments, the catheter or cannula may be located at more than one site in the spine for multi-site delivery. The viral particles encoding may be delivered in a continuous and/or bolus infusion. Each site of delivery may be a different dosing regimen or the same dosing regimen may be used for each site of delivery. In some embodiments, the sites of delivery may be in the cervical and the lumbar region. In some embodiments, the sites of delivery may be in the cervical region. In some embodiments, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the delivery method and duration is chosen to provide broad transduction in the spinal cord. In some embodiments, intrathecal delivery is used to provide broad transduction along the rostral-caudal length of the spinal cord. In some embodiments, multi-site infusions provide a more uniform transduction along the rostral-caudal length of the spinal cord.

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In some embodiments, the AAV particles may be delivered to bypass anatomical blockages such as, but not limited to the blood brain barrier.

In some embodiments, the AAV particles may be formulated and delivered to a subject by a route which increases the speed of drug effect as compared to oral delivery.

In some embodiments, the AAV particles may be delivered by a method to provide uniform transduction of the spinal cord and dorsal root ganglion (DRG). In some embodiments, the AAV particles may be delivered using intrathecal infusion.

In some embodiments, a subject may be administered the AAV particles described herein using a bolus infusion. As used herein, a “bolus infusion” means a single and rapid infusion of a substance or composition.

In some embodiments, the AAV particles encoding a SOD1 targeting siRNA may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours or days. The infusion rate may be changed depending on the subject, distribution, formulation or another delivery parameter known to those in the art.

In some embodiments, if continuous delivery (continuous infusion) of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume, AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3: 16046). In some cases, the AAV particles may be injected into the cervical subpial space. In some cases, the AAV particles may be injected into the thoracic subpial space.

In some embodiments, the AAV particles may be delivered by direct injection to the CNS of a subject. In some embodiments, direct injection is intracerebral injection, intraparenchymal injection, intrathecal injection, intra-cisterna magna injection, or any combination thereof. In some embodiments, direct injection to the CNS of a subject comprises convection enhanced delivery (CED). In some embodiments, administration comprises peripheral injection. In some embodiments, peripheral injection is intravenous injection.

In some embodiments, the AAV particles may be delivered to a subject in order to decrease SOD1 activity in the caudate-putamen, thalamus, superior colliculus, cortex, and/or corpus callosum as compared to endogenous levels. In some embodiments, the AAV particles may be delivered to a subject in order to decrease SOD1 activity in the motor and sensory cranial nerves, as well as neurons of the cortex, the hippocampus, and amygdala as compared to endogenous levels. The decrease may be 0.1× to 5×, 0.5× to 5×, 1x to 5×, 2× to 5×, 3× to 5×, 4× to 5×, 0.1× to 4×, 0.5× to 4×, 1× to 4×, 2× to 4×, 3× to 4×, 0.1× to 3×, 0.5× to 3×, 1× to 3×, 2× to 3×, 0.1× to 2×, 0.5× to 2×, 0.1× to 1×, 0.5× to 1×, 0.1× to 0.5×, 1× to 2×, 0.1×, 0.2×, 0.3×, 0.4×, 0.5×, 0.6×, 0.7×, 0.8×, 0.9×, 1.0×, 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9×, 4.0×, 4.1×, 4.2×, 4.3×, 4.4×, 4.5×, 4.6×, 4.7×, 4.8×, 4.9× or more than 5× as compared to endogenous levels.

In some embodiments, the AAV particles may be delivered to a subject in order to decrease SOD1 activity levels in the caudate, putamen, thalamus, superior colliculus, cortex, and/or corpus callosum by transducing cells in these CNS regions. In some embodiments, the AAV particles may be delivered to a subject in order to transducing cells in the motor and sensory cranial nerves, as well as neurons of the cortex, the hippocampus, and amygdala by transducing cells in these CNS regions. Transduction may also be referred to as the amount of cells that are positive for the SOD1 targeting siRNA. The transduction may be greater than or equal to 1%, 5%, 10%, 15%, 20%, 25%, 30%, 35%, 40%, 45%, 50%, 55%, 60%, 65%, 70%, 75%, 80%, 85%, 90%, 95%, or 99% of cells in these CNS regions.

In some embodiments, delivery of AAV particles comprising a viral genome encoding the SOD1 targeting siRNA described herein to motor neurons (e.g., motor neurons or ventral horn motor neurons); glial cells including oligodendrocyte, astrocyte and microglia; and/or other cells surrounding neurons such as T cells, will lead to an decrease in the activity of SOD1. The decreased activity of SOD1 may lead to improved survival and function of various cell types in these CNS regions and subsequent improvement of SOD1-related disorder symptoms.

In particular embodiments, the AAV particles may be delivered to a subject in order to establish widespread distribution of the SOD1 targeting siRNA throughout the nervous system by administering the AAV particles to the thalamus of the subject.

In some embodiments, the composition of the present disclosure for treating ALS is administered to the subject in need intravenously, intramuscularly, subcutaneously, intraperitoneally, intrathecally, intraparenchymally (CNS, brain, and/or spinal cord) and/or intraventricularly, allowing the siRNA duplexes or vectors comprising the siRNA duplexes to pass through one or both the blood-brain barrier and the blood spinal cord barrier. In some aspects, the method includes administering (e.g., intraparenchymally administering, intraventricularly administering and/or intrathecally administering) directly to the central nervous system (CNS) of a subject (using, e.g., an infusion pump and/or a delivery scaffold) a therapeutically effective amount of a composition comprising at least one siRNA duplex targeting SOD1 gene or AAV particles comprising at least one siRNA duplex targeting SOD1 gene, silencing/suppressing SOD1 gene expression, and reducing one or more symptoms of ALS in the subject such that ALS is therapeutically treated.

In some embodiments, the composition of the present disclosure for treating ALS is administered to the subject in need intraparenchymally (CNS, brain, and/or spinal cord), allowing the siRNA duplexes or vectors comprising the siRNA duplexes to pass through one or both the blood-brain barrier and the blood spinal cord barrier.

In certain aspects, the symptoms of ALS including motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present disclosure is applied to one or both of the brain and the spinal cord. In other aspects, one or both of muscle coordination and muscle function are improved. In other aspects, the survival of the subject is prolonged.

### Dosing

In some aspects, the present disclosure provides methods comprising administering viral vectors and their payloads in accordance with the disclosure to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition associated with SOD1). In some embodiments, the disease, disorder, and/or condition is SOD1-related disorders. The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific peptide(s) employed; the duration of the treatment; drugs used in combination or coincidental with the specific compound employed; and like factors well known in the medical arts.

In certain embodiments, AAV particle pharmaceutical compositions in accordance with the present disclosure may be administered at dosage levels sufficient to deliver SOD1 targeting siRNAs from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to VG or viral genomes per kg or into total viral genomes administered by one of skill in the art.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In some embodiments, delivery of the AAV particles described herein results in minimal serious adverse events (SAEs) as a result of the delivery of the AAV particles.

In some embodiments, delivery of AAV particle pharmaceutical compositions in accordance with the present disclosure to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.3×10¹³, 3×10¹³, 4×10¹³, 5×10¹³, 6×10¹³, 7×10¹³, 8×10¹³, 9×10¹³, 1×10¹⁴, 1.9×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In some embodiments, delivery of AAV particle pharmaceutical compositions in accordance with the present disclosure to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In some embodiments, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

### Devices

As used herein, the term “device” refers to any article constructed or modified to suit a particular purpose, such as facilitating the delivery of the pharmaceutical compositions to a subject or the detection of the administered pharmaceutical compositions in a subject.

In some embodiments, the devices may be utilized for intraparenchymal injection of the pharmaceutical compositions. Devices may also be used to administer the pharmaceutical compositions to the spinal cord.

In some embodiments, the device may be a custom floating cannula. In certain embodiments, the custom infusion cannula with a narrow diameter is used for the injections. The cannula may include a 30-gauge beveled needle of fixed length connected to a 30-gauge flexible silastic tubing of variable length. The distal end may be fitted with a Hamilton luer lock, which, in turn, may be attached to a microinjector pump. The proximal silastic tubing may be ensheathed within a 24-gauge rigid outer cannula that is seated on the proximal end of the injection needle flange. The flange seats the outer cannula and may serve as a depth stop for the injection needle

In certain embodiments, the device may be an intraspinal cannula. The intraspinal cannula may include proximal syringe connection and a distal tip. The proximal syringe connection comprises a female luer lock syringe connector which may be connected to a 3-20′ cannula with protective sheathing. The cannula may include a single internal lumen from the distal tip to the syringe. The cannula may include a 4-6″ flexible portion near the distal tip. The distal tip includes a flange/depth stop and a blunt rigid tip. The intraspinal cannula may also include a mechanism for attachment to the subject.

In certain embodiments, the device may be a complex stereotactic frame.

In certain embodiments, the device may be a simplified stereotactic frame.

In certain embodiments, the pharmaceutical compositions may be delivered without a frame.

In certain embodiments, the device may be magnetic resonance imager. Such imagers when used in conjunction with contrast agents such as Gadolinium can detect the administered pharmaceutical compositions in a subject.

In certain embodiments, any of the devices described herein may be combined to deliver and/detect the administered pharmaceutical compositions.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

The therapeutic agents may be approved by the US Food and Drug Administration or may be in clinical trial or at the preclinical research stage. The therapeutic agents may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Measurement of Expression

In some embodiments, the administration further comprises evaluating, e.g., measuring, the level of SOD1 expression, e.g., SOD1 gene, SOD1 mRNA, and/or SOD1 protein expression, in the subject, e.g., in a cell, tissue, or fluid, of the subject. The measuring of the level of SOD1 expression may be performed prior to, during, or subsequent to treatment with the AAV particle.

Expression of the SOD1 targeting siRNA from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, PCR, and/or in situ hybridization (ISH). In some embodiments, transgenes encoding the SOD1 targeting siRNA delivered in different AAV capsids may have different expression levels in different CNS tissues.

In certain embodiments, the SOD1 targeting siRNA is detectable by Western blot.

### IX. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or SOD1 targeting siRNAs of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more SOD1 targeting polynucleotide. These devices contain in a stable formulation the viral vectors which may be immediately delivered to a subject in need thereof, such as a human patient.

Devices for administration may be employed to deliver the viral vectors encoding the SOD1 targeting polynucleotide of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure.

### X. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV capsid polypeptide, e.g., a parent capsid sequence with at least one peptide insert. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide present, e.g., inserted into, the capsid to enhance tropism for a desired target tissue. It is to be understood that reference to the AAV particle of the disclosure also includes pharmaceutical compositions thereof, even if not explicitly recited.

Administered in combination: As used herein, the term “administered in combination” or “delivered in combination” or “combined administration” refers to exposure of two or more agents (e.g., AAV) administered at the same time or within an interval such that the subject is at some point in time exposed to both agents and/or such that there is an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatuses in administration. In some embodiments, the administration of individual doses of one or more compounds and/or compositions of the present disclosure, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.25%, 0.1%, or less in either direction (greater than or less than) of the stated reference value unless otherwise stated or otherwise evident from the context (except where such number would exceed 100% of a possible value).

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95%, 90%, 85%, 80%, 70%, 60% or less. For example, gaps or discontinuities may be present in the capsid so long as the viral genome is retained in the capsid, e.g., prior to entry into a cell.

Central Nervous System or CNS: As used herein, “central nervous system” or “CNS” refers to one of the two major subdivisions of the nervous system, which in vertebrates includes the brain and spinal cord. The central nervous system coordinates the activity of the entire nervous system.

Cervical Region: As used herein, “cervical region” refers to the region of the spinal cord comprising the cervical vertebrae C1, C2, C3, C4, C5, C6, C7, and C8.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non-limiting examples of structures in the brain include, forebrain, midbrain, hindbrain, diencephalon, telencephalon, myelencephalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, putamen, striatum, ventricles and sub-structures thereof.

CNS Cells: As used herein, “CNS cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, pseudounipolar cells, pyramidal cells, basket cells, stellate cells, Purkinje cells, Betz cells, amacrine cells, granule cell, ovoid cell, medium aspiny neurons and large aspiny neurons.

Complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In some embodiments, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present disclosure, e.g., an AAV viral particle) to targeted cells.

Delivery route: As used herein, the term “delivery route” and the synonymous term “administration route” refers to any of the different methods for providing a therapeutic agent to a subject. Routes of administration are generally classified by the location at which the substance is applied and may also be classified based on where the target of action is. Examples include, but are not limited to: intravenous administration, subcutaneous administration, oral administration, parenteral administration, enteral administration, topical administration, sublingual administration, inhalation administration, and injection administration, or other routes of administration described herein.

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats amyotrophic lateral sclerosis (ALS) and related disorders, an effective amount of an agent is, for example, an amount sufficient to achieve treatment, as defined herein, of a SOD1-related disorder as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and/or (5) post-translational modification of a polypeptide or protein.

Excipient: As used herein, the term “excipient” refers to an inactive substance that serves as the vehicle or medium for an active pharmaceutical agent or other active substance.

Formulation: As used herein, a “formulation” includes at least a compound and/or composition of the present disclosure (e.g., a vector, AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50, or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Humanized: As used herein, the term “humanized” refers to a non-human sequence of a polynucleotide or a polypeptide which has been altered to increase its similarity to a corresponding human sequence.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molecular Biol., 215, 403 (1990)).

Inhibit expression of a gene: As used herein, the phrase “inhibit expression of a gene” means to cause a reduction in the amount of an expression product of the gene. The expression product can be a RNA molecule transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Lumbar Region: As used herein, the term “lumbar region” refers to the region of the spinal cord comprising the lumbar vertebrae L1, L2, L3, L4, and L5.

miR binding site series: As used herein, the “miR binding site series” or the “miR binding site” includes an RNA sequence on the RNA transcript produced by transcribing the AAV viral genome. The “miR binding site series” or the “miR binding site” also includes the DNA sequence corresponding to the RNA sequence, in that they differ only by the T in DNA and the U in RNA. The reverse complement of such DNA is the coding sequence for the RNA sequence. That is, in some embodiments, in an expression cassette containing a DNA positive strand, the miR binding site sequence is the reverse complement of the miRNA to which it binds.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides. One or more mutations may result in a “mutant,” “derivative,” or “variant,” e.g., of a nucleic acid sequence or polypeptide or protein sequence.

Variant: The term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: The term “functional variant” refers to a polypeptide variant or a polynucleotide variant that has at least one activity of the reference sequence.

Insertional Variant: “Insertional variants” when referring to polypeptides are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid.

Nucleic acid: As used herein, the terms “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide that is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single-stranded RNA.

Neurodegeneration: As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. In some neurological disorders, mainly one type of neuron cells is degenerative, for example, motor neuron degeneration in ALS.

Operably linked. As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise a component for viral expression in a viral replication cell.

Pharmaceutically acceptable: The phrase “pharmaceutically acceptable” is employed herein to refer to those compounds, materials, compositions, and/or dosage forms which are suitable for use in contact with the tissues of human beings and animals.

Pharmaceutically acceptable excipients: As used herein, the term “pharmaceutically acceptable excipient,” as used herein, refers to any ingredient other than active agents (e.g., as described herein) present in pharmaceutical compositions t that can function as vehicles for suspending and/or dissolving active agents.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of each of which are incorporated herein by reference in their entirety.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Promoter: As used herein, the term “promoter” refers to a nucleic acid site to which a polymerase enzyme will bind to initiate transcription (DNA to RNA) or reverse transcription (RNA to DNA).

Purified: As used herein, the term “purify” means to make substantially pure or clear from one or more unwanted components, material defilement, admixture or imperfection. “Purified” refers to the state of being pure. “Purification” refers to the process of making pure. As used herein, a substance is “pure” if it is substantially free of (substantially isolated from) one or more components, e.g., one or more components found in a native context.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may comprise the N- and/or C-termini as well as surrounding amino acids. In some embodiments, N- and/or C-terminal regions comprise from about 3 amino acids to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA agent: As used herein, the term “RNA agent,” refers to an agent that contains RNA as that term is defined herein, and which mediates the targeted cleavage of an RNA transcript, e.g., via an RNA-induced silencing complex (RISC) pathway. In some embodiments, the RNA agent is a single-stranded antisense RNA molecule, a single-stranded siRNA, or a double-stranded RNA (e.g., a siRNA duplex), e.g., as described herein.

RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen).

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand. According to the present disclosure, recombinant AAV genome may encode a sense and/or antisense strand.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequences” refers to a sequence which can direct the transport or localization.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

siRNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex. According to the present disclosure, recombinant AAV genome may encode one or more RNAi molecules such as an siRNA, shRNA, microRNA or precursor thereof.

Spacer: As used herein, a “spacer” is generally any selected nucleic acid sequence of, e.g., 1, 2, 3, 4, 5, 6, 7, 8, 9, or 10 nucleotides in length, which is located between two or more consecutive miR binding site sequences.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “target cells” or “targeted cells” refers to any one or more cells of interest. The cells may be found in vitro, in vivo, in situ or in the tissue or organ of an organism. The organism may be an animal, a mammal, a human and/or a patient. The target cells may be CNS cells or cells in CNS tissue.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Thoracic Region: As used herein, a “thoracic region” refers to a region of the spinal cord comprising the thoracic vertebrae T1, T2, T3, T4, T5, T6, T7, T8, T9, T10, T11, and T12.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence(s). Such parent or reference AAV sequences may serve as an original, second, third or subsequent sequence for engineering vectors.

Viral construct vector: As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” or “vector genome” is a polynucleotide comprising at least one inverted terminal repeat (ITR) and at least one encoded payload. A viral genome encodes at least one copy of the payload.

The present disclosure is further illustrated by the following non-limiting examples.

### EXAMPLES

### Example 1. SOD1 Targeting Polynucleotide Design (siRNA)

siRNA design is carried out to identify siRNAs targeting human SOD1 gene. The design uses the SOD1 transcripts from human (GenBank access No. NM_000454.4 (SEQ ID NO: 110)), cynomolgus (GenBank access No. NM_001285406.1 (SEQ ID NO: 111)), rhesus SOD1 transcript (GenBank access No. NM_001032804.1 (SEQ ID NO: 111)), and Sus scrofa (GenBank access No. NM_001190422.1 (SEQ ID NO: 112)), respectively (Table 19). The siRNA duplexes are designed with 100% identity to the human SOD1 transcript for positions 2-18 of the antisense strand, and partial or 100% identity to the non-human SOD1 transcript for positions 2-18 of the antisense strand. In all siRNA duplexes, position 1 of the antisense strand is engineered to a U and position 19 of the sense strand is engineered to a C, in order to unpair the duplex at this position.

TABLE 19
SOD1 gene sequences

| SOD1 | Access | SEQ ID | |
|---|---|---|---|
| transcripts | No. | NO. | Sequence |
| Human (Homo | NM_ | 110 | GTTTGGGGCCAGAGTGGGCGAGGCGCGGAGGTCTGGCCTATAAAGT |
| sapiens) SOD1 | 000454.4 | | AGTCGCGGAGACGGGGTGCTGGTTTGCGTCGTAGTCTCCTGCAGCG |
| cDNA (981 bp) | | | TCTGGGGTTTCCGTTGCAGTCCTCGGAACCAGGACCTCGGCGTGGC |
| | | | CTAGCGAGTTATGGCGACGAAGGCCGTGTGCGTGCTGAAGGGCGAC |
| | | | GGCCCAGTGCAGGGCATCATCAATTTCGAGCAGAAGGAAAGTAATG |
| | | | GACCAGTGAAGGTGTGGGGAAGCATTAAAGGACTGACTGAAGGCCT |
| | | | GCATGGATTCCATGTTCATGAGTTTGGAGATAATACAGCAGGCTGT |
| | | | ACCAGTGCAGGTCCTCACTTTAATCCTCTATCCAGAAAACACGGTG |
| | | | GGCCAAAGGATGAAGAGAGGCATGTTGGAGACTTGGGCAATGTGAC |
| | | | TGCTGACAAAGATGGTGTGGCCGATGTGTCTATTGAAGATTCTGTG |
| | | | ATCTCACTCTCAGGAGACCATTGCATCATTGGCCGCACACTGGTGG |
| | | | TCCATGAAAAAGCAGATGACTTGGGCAAAGGTGGAAATGAAGAAAG |
| | | | TACAAAGACAGGAAACGCTGGAAGTCGTTTGGCTTGTGGTGTAATT |
| | | | GGGATCGCCCAATAAACATTCCCTTGGATGTAGTCTGAGGCCCCTT |
| | | | AACTCATCTGTTATCCTGCTAGCTGTAGAAATGTATCCTGATAAAC |
| | | | ATTAAACACTGTAATCTTAAAAGTGTAATTGTGTGACTTTTTCAGA |
| | | | GTTGCTTTAAAGTACCTGTAGTGAGAAACTGATTTATGATCACTTG |
| | | | GAAGATTTGTATAGTTTTATAAAACTCAGTTAAAATGTCTGTTTCA |
| | | | ATGACCTGTATTTTGCCAGACTTAAATCACAGATGGGTATTAAACT |
| | | | TGTCAGAATTTCTTTGTCATTCAAGCCTGTGAATAAAAACCCTGTA |
| | | | TGGCACTTATTATGAGGCTATTAAAAGAATCCAAATTCAAACTAAA |
| | | | AAAAAAAAAAAAAAA |
| Cynomolgus | NM_ | 111 | ATGGCGATGAAGGCCGTGTGCGTGTTGAAGGGCGACAGCCCAGTGC |
| (Macaca | 001285406.1 | | AGGGCACCATCAATTTCGAGCAGAAGGAAAGTAATGGACCAGTGAA |
| fascicularis) | | | GGTGTGGGGAAGCATTACAGGATTGACTGAAGGCCTGCATGGATTC |
| SOD1 cDNA | | | CATGTTCATCAGTTTGGAGATAATACACAAGGCTGTACCAGTGCAG |
| (465 bp) | | | GTCCTCACTTTAATCCTCTATCCAGACAACACGGTGGGCCAAAGGA |
| | | | TGAAGAGAGGCATGTTGGAGACCTGGGCAATGTGACTGCTGGCAAA |
| | | | GATGGTGTGGCCAAGGTGTCTTTCGAAGATTCTGTGATCTCGCTCT |
| | | | CAGGAGACCATTCCATCATTGGCCGCACATTGGTGGTCCATGAAAA |
| | | | AGCAGATGACTTGGGCAAAGGTGGAAATGAAGAAAGTAAAAAGACA |
| | | | GGAAACGCTGGAGGTCGTCTGGCTTGTGGTGTAATTGGGATCGCCC |
| | | | AATAA |
| rhesus (Macaca | NM_ | 111 | ATGGCGATGAAGGCCGTGTGCGTGTTGAAGGGCGACAGCCCAGTGC |
| mulatta) SOD1 | 001032804.1 | | AGGGCACCATCAATTTCGAGCAGAAGGAAAGTAATGGACCAGTGAA |
| cDNA (465 bp) | | | GGTGTGGGGAAGCATTACAGGATTGACTGAAGGCCTGCATGGATTC |
| | | | CATGTTCATCAGTTTGGAGATAATACACAAGGCTGTACCAGTGCAG |
| | | | GTCCTCACTTTAATCCTCTATCCAGACAACACGGTGGGCCAAAGGA |
| | | | TGAAGAGAGGCATGTTGGAGACCTGGGCAATGTGACTGCTGGCAAA |
| | | | GATGGTGTGGCCAAGGTGTCTTTCGAAGATTCTGTGATCTCGCTCT |
| | | | CAGGAGACCATTCCATCATTGGCCGCACATTGGTGGTCCATGAAAA |
| | | | AGCAGATGACTTGGGCAAAGGTGGAAATGAAGAAAGTAAAAAGACA |
| | | | GGAAACGCTGGAGGTCGTCTGGCTTGTGGTGTAATTGGGATCGCCC |
| | | | AATAA |
| Pig (Sus scrofa) | NM_ | 112 | CGTCGGCGTGTACTGCGGCCTCTCCCGCTGCTTCTGGTACCCTCCC |
| SOD1 cDNA | 001190422.1 | | AGCCCGGACCGGAGCGCGCCCCCGCGAGTCATGGCGACGAAGGCCG |
| (658 bp) | | | TGTGTGTGCTGAAGGGCGACGGCCCGGTGCAGGGCACCATCTACTT |
| | | | CGAGCTGAAGGGAGAGAAGACAGTGTTAGTAACGGGAACCATTAAA |
| | | | GGACTGGCTGAAGGTGATCATGGATTCCATGTCCATCAGTTTGGAG |
| | | | ATAATACACAAGGCTGTACCAGTGCAGGTCCTCACTTCAATCCTGA |
| | | | ATCCAAAAAACATGGTGGGCCAAAGGATCAAGAGAGGCACGTTGGA |
| | | | GACCTGGGCAATGTGACTGCTGGCAAAGATGGTGTGGCCACTGTGT |
| | | | ACATCGAAGATTCTGTGATCGCCCTCTCGGGAGACCATTCCATCAT |
| | | | TGGCCGCACAATGGTGGTCCATGAAAAACCAGATGACTTGGGCAGA |
| | | | GGTGGAAATGAAGAAAGTACAAAGACGGGAAATGCTGGAAGTCGTT |
| | | | TGGCCTGTGGTGTAATTGGGATCACCCAGTAAACATTCCCTCATGC |
| | | | CATGGTCTGAATGCCAGTAACTCATCTGTTATCTTGCTAGTTGTAG |
| | | | TTGTAGAAATTTAACTTGATAAACATTAAACACTGTAACCTTAAAA |
| | | | AAAAAAAAAAAAAA |

### Example 2. NHP High-Throughput Screen of TRACER AAV Libraries

A TRACER based method as described in WO2020072683, the contents of which are herein incorporated by reference in their entirety, was adapted for use in non-human primates (NHP), as described in WO 2021/202651 and PCT/US2021/025061, the contents of which are herein incorporated by reference in their entirety. An orthogonal evolution approach was combined with a high throughput screening by NGS in NHP as described in WO 2021/202651 and PCT/US2021/025061, the contents of which are herein incorporated by reference in their entirety. Briefly, AAV9/AAV5 starting libraries, driven by synapsin or GFAP promoters were administered to non-human primate (NHP) intravenously for in vivo AAV selection (biopanning), performed iteratively. All libraries were injected intravenously at a dose of 1e14VG per animal (approximately 3e13 VG/kg). Orthogonally, biopanning was conducted in hBMVEC cells using the same starting libraries. In the second round of biopanning in NHP, only libraries driven by the synapsin promoter were used. After a period, (e.g., 1 month) RNA was extracted from nervous tissue, e.g., brain and spinal cord. Following RNA recovery and RT-PCR amplification, a systematic NGS enrichment analysis was performed, and the peptides shown in Table 2 and 20 identified. Capsid enrichment ratio, including calculating the ratio of, e.g., P2/P1 reads and comparison to a benchmark (e.g., AAV9) was evaluated.

Candidate library enrichment data in P3 NHP brain for the peptides identified, over benchmark AAV9, are shown in Table 20. Data are provided as fold enrichment. Fifty-one variants showed greater than 10-fold enrichment over AAV9. Variants with 0.0 enrichment over AAV9 are not included in Table 20.

TABLE 20
NHP NGS AAV9 Enrichment

| | Fold | | Fold |
|---|---|---|---|
| Peptide | enrichment | Peptide | enrichment |
| Sequence | over AAV9 | Sequence | over AAV9 |
| PLNGAVHLYA | 473.7 | AQAGEQSTRL | 16.1 |
| AQARDSPKGW | 214 | AQASNDVGRA | 15.4 |
| LTNGAVRDRP | 134.4 | AQATFTASEY | 15.3 |
| VQAFTHDSRG | 88.6 | AKAHAGTIYS | 14.9 |
| AQAYSTDVRM | 84.8 | AQARTIDQCC | 14.8 |
| AQAYSTDVRI | 83.8 | AQEYNSNPKA | 14.5 |
| AQAFTAAERM | 74.9 | AQVVDNSTHA | 14.5 |
| AQTHLQIGVA | 54.6 | AQATLSVPLK | 14.4 |
| AQSNAVLSLA | 51.6 | AQIVMNSLKA | 12.5 |
| AQAYSTDERM | 41.4 | AQATMSQTMA | 12.5 |
| AQAYSTDVRL | 31.7 | AQALTQDERW | 12 |
| AQATVSTLRM | 31.5 | AQAQLSTLRP | 11.6 |
| AQAYSTDERK | 31.2 | AQVVMGISVA | 11.4 |
| AQAYSTDMRM | 30.4 | AQAYTTDVRM | 11.4 |
| VVNGAVLHVA | 29.8 | AQHIDSMRPP | 11.3 |
| AQAYSTDVTM | 29.7 | AQASTGTLRL | 11.1 |
| AQAHLQIGVA | 23 | AQHRALDYYA | 11 |
| FLDPAVSSKA | 22.6 | AQARESPRGL | 10.9 |
| AQAYVSTLRM | 21.9 | AQALLAGTRV | 10.7 |
| AQAQTGPPLK | 20.1 | TKIQAVPWNA | 10.7 |
| EQASRLPTPG | 20 | AQASLSSTRP | 10.6 |
| AQASVSTMRM | 19.7 | AQAMGSRSDQ | 10.4 |
| TDYSAVRLGA | 18 | AQAAQGTYRG | 10.3 |
| TQAYSTDVRM | 17.9 | SQENAVFSKA | 10.3 |
| AQALPSNERL | 17.4 | AQAYGLPKGP | 8.4 |
| AQAYSTDVRT | 16.4 | GGTLAVVSLA | 6.9 |
| AQSSLPEMVA | 16.2 | AQAYVSSVKM | 5.2 |

A subset of the peptide variants from the NHP biopanning showed a very strong and consistent enrichment over AAV9 and PHP.B controls. Further, the peptide of SEQ ID NO: 1725 not only showed a strong enrichment over AAV9 in the brain, but also in the spinal cord, as it led to a 125.6 fold enrichment over AAV9 in the spinal cord. Following the removal of the least reliable variants, a set of 22 variants with enrichment factors ranging from 7-fold to >400-fold over AAV9 was identified. These were cross-referenced to a non-synthetic PCR-amplified library screened in parallel and 12 candidates showed reliable enrichment and high consistency in both assays. Of these, 5 candidates with the highest enrichment scores in both assays and the highest consistency across animals and tissues were retained for individual evaluation. Candidate capsids were labeled TTD-001, TTD-002, TTD-003, TTD-004 and TTD-005 as shown in Table 4 above.

After 3 rounds of screening of AAV9 peptide insertion library in NHP, many capsids outperformed their parental capsid AAV9 in penetration of the blood brain barrier (BBB). Some of the capsids comprising a peptide showed high enrichment scores and high consistency both across different brain tissue samples from the same animal and across different animals. Consistency in both NNK and NNM codons was also observed. 22 capsid variants exhibited enrichment factors ranging from 7-fold to >400-fold over AAV9 in the brain tissues. A majority of these variants also demonstrated high enrichment factors up to 125-fold over AAV9 in the spinal cord. Of these, 5 candidates with diverse inserted sequences were selected for further evaluation as individual capsids.

### Example 3. Individual Capsid Characterization

The goal of these experiments was to determine the transduction level and the spatial distribution of each of the 5 capsid candidates selected from the study described in Example 2 relative to AAV9 following intravascular infusion in NHPs (cynomolgus macaque). The 5 selected capsid candidates were TTD-001 (SEQ ID NO: 3623 and 3636, comprising SEQ ID NO: 3648), TTD-002 (SEQ ID NO: 3624, 3625, and 3637, comprising SEQ ID NO: 3649), TTD-003 (SEQ ID NO: 3626 and 3638, comprising SEQ ID NO: 3650), TTD-004 (SEQ ID NO: 3627 and 3639, comprising SEQ ID NO: 3651) and TTD-005 (SEQ ID NO: 3628 and 3640, comprising SEQ ID NO: 3652) as outlined in Table 4 above.

AAV particles were generated with each of these 5 capsids encapsulating a transgene encoding a payload fused to an HA tag (payload-HA) and driven by a full-length CMV/chicken beta actin promoter by triple transfection in HEK293T cells and formulated in a pharmaceutically acceptable solution. Each test capsid and AAV9 control were tested by intravenously providing two (2) NHP females the AAV particle formulation at a dose of 2e13 VG/kg. The in-life period was 14 days and then a battery of CNS and peripheral tissues were collected for quantification of transgene mRNA, transgene protein and viral DNA (biodistribution). Samples were also collected, fixed and paraffin embedded for immunohistochemical staining.

In a first pass screening of RNA quantification by qRT-PCR and RT-ddPCR, total RNA was extracted from 3-mm punches from various areas of the brain (cortex, striatum, hippocampus, cerebellum), spinal cord sections, liver and heart, and analyzed by qRT-PCR using a proprietary Taqman set specific for the synthetic CAG exon-exon junction. Cynomolgus TBP (TATA box-binding protein) was used as a housekeeping gene.

TRACER capsids showed an increase in RNA expression in all brain regions relative to AAV9 in at least one animal. The highest and most consistent increase in brain transduction was observed with capsids TTD-003 and TTD-004 (8- to 200-fold depending in various anatomical locations). In this initial screening TTD-001 was not assessed due to staggered animal dosing. An approximate 10- to 12-fold increase was consistently observed in whole brain slices (equivalent to an average of multiple regions), which was consistent with the values indicated in a next-generation sequencing (NGS) assay. In order to increase data robustness, droplet digital RT-PCR (ddPCR) was performed in parallel to qRT-PCR and confirmed the trends indicated by the qPCR data.

Interestingly, RNA quantification performed in the spinal cord and dorsal root ganglia indicated important differences between the capsid variants. The spinal cord transduction profile was consistent with the brain, with a strong and consistent increase with TTD-003 and TTD-004 capsids, but interestingly the DRG transduction suggested a substantial detargeting of the TTD-004 capsid, whereas the TTD-003 capsid showed a strongly increased RNA expression.

Total DNA was extracted from the same brain tissues as RNA, and biodistribution was measured by ddPCR using a Taqman set specific for the CMV promoter sequence. The RNAseP gene was used as a copy number reference. Vector genome (VG) per cell values were determined both by qPCR and ddPCR. Increased biodistribution was observed for the TTD-004 capsid in most brain regions, but surprisingly none of the other candidates showed a significant increase by comparison with AAV9. This apparent contradiction with the RNA quantification data could suggest that some capsids may present improved properties over AAV9 in post-attachment mechanisms rather than strict vector translocation in CNS parenchyma. Interestingly, DNA analysis confirmed the substantial detargeting of TTD-004 capsid from the DRG.

To further explore the behavior of capsid variant TTD-004, viral genome (VG) quantification was completed from tissues collected from heart atrium, heart ventricle, quadriceps muscle, liver (left and right) and diaphragm and compared to vector genome presence as delivered by AAV9 in the same tissues.

For TTD-003 and TTD-004 initial immunohistochemical analyses demonstrated the presence of payload-HA to a greater extent than seen with AAV9 delivery in cerebellar tissue, including in the dentate nucleus. Immunohistochemistry confirmed the de-targeting of the dorsal root ganglia for capsid variant TTD-004 as compared to TTD-003 and AAV9.

Data for each of the variants as described above were compiled as an average mRNA (fold over TBP) or DNA (VG per cell) quantification per capsid variant per tissue as shown in Table 21 below.

TABLE 21
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal | 0.000325065 | 2.7232575 | 0.000768179 | 0.006268831 | 0.007076252 | 0.002204024 |
| | Cortex | | | | | | |
| mRNA | Sensory | 0.001486245 | 3.400055 | 0.00417739 | 0.006788644 | 0.010976612 | 0.004139604 |
| | Cortex | | | | | | |
| mRNA | Motor Cortex | 0.00063318 | 9.00819 | 0.001050247 | 0.009954825 | 0.010522399 | 0.002942249 |
| mRNA | Putamen | 0.000612759 | 3.557205 | 0.001395549 | 0.011832671 | 0.011476176 | 0.001150153 |
| mRNA | Thalamus | 0.002610992 | 2.863635 | 0.013937891 | 0.101411445 | 0.07565653 | 0.01100289 |
| mRNA | Cerebellar | 0.00133497 | 1.3439 | 0.008517779 | 0.006396677 | 0.012964181 | 0.004382119 |
| | Cortex | | | | | | |
| mRNA | Dentate | 0.001364954 | 0.963955 | — | — | — | — |
| | Nucleus | | | | | | |
| mRNA | Caudate | 0.000352281 | 1.3026 | — | 0.003259804 | 0.00634117 | — |
| mRNA | Hippocampus | 0.000311824 | 0.407015 | — | — | — | — |
| mRNA | SC-cervical | 0.012205449 | 11.877762 | 0.022004264 | 0.026994764 | 0.088316491 | 0.005773054 |
| mRNA | SC-Thoracic | 0.048833465 | 2.9974295 | 0.004360318 | 0.035118928 | 0.020543776 | 0.005629959 |
| mRNA | SC-Lumbar | 0.029887407 | 7.969603 | 0.056231995 | 0.016033388 | 0.047713563 | 0.026324154 |
| mRNA | DRG- | 0.74570895 | 9.274951 | 0.007897714 | 2.47872652 | 0.280868887 | 0.008122233 |
| | cervical | | | | | | |
| mRNA | DRG- | 0.5559061 | 5.22606 | 0.006456564 | 8.721845271 | 0.104701895 | — |
| | Thoracic | | | | | | |
| mRNA | DRG- | 1.089758 | 17.308436 | 0.008247771 | 2.271300217 | 0.426704698 | 0.119974244 |
| | Lumbar | | | | | | |
| mRNA | Lung | 0.004807149 | 0.000546842 | — | — | 0.013744781 | — |
| mRNA | Pancreas | — | — | — | — | — | — |
| mRNA | Colon | 0.017962678 | 0.005041385 | — | — | 0.183862903 | — |
| mRNA | Kidney | 0.043825993 | 0.006649157 | — | — | 0.041234576 | — |
| mRNA | Liver | 0.674478605 | 0.253188648 | — | — | 2.578654807 | — |
| mRNA | Adrenal | — | — | — | — | — | — |
| mRNA | Spleen | 0.014066875 | 0.000955981 | — | — | 0.013435626 | — |
| mRNA | Heart | 1.323389668 | 0.132477314 | — | — | 5.587929805 | — |
| mRNA | Quadriceps | 0.116623509 | — | — | — | 4.527799743 | — |
| mRNA | Diaphragm | 0.250001109 | — | — | — | 1.936435215 | — |
| DNA | Frontal | 0.07713 | 2.104843 | 0.10252 | 0.068367 | 0.380429 | 0.1257545 |
| | Cortex | | | | | | |
| DNA | Sensory | 0.093003 | 2.679886 | 0.07443 | 0.034016 | 0.2670975 | 0.132503 |
| | Cortex | | | | | | |
| DNA | Motor Cortex | 0.08796 | 4.3437625 | 0.0913085 | 0.094401 | 0.318999 | 0.1110695 |
| DNA | Putamen | 0.0581365 | 3.07904 | 0.12326 | 0.1497635 | 0.2731175 | 0.0715295 |
| DNA | Thalamus | 0.0524055 | 2.076863 | 0.0664225 | 0.090511 | 0.214999 | 0.086863 |
| DNA | Cerebellar | 0.014238 | 0.186361 | 0.0092915 | 0.009578 | 0.0356345 | 0.0128655 |
| | Cortex | | | | | | |
| DNA | Dentate | 0.025042 | 0.1861975 | 0.210238 | 0.041906 | 0.106107 | 0.055287 |
| | Nucleus | | | | | | |
| DNA | Caudate | 0.079294 | 3.9433175 | — | 0.0529005 | 0.2451035 | — |
| DNA | Hippocampus | 0.095436 | 1.760891 | 0.205433 | 0.368645 | 1.335324 | 0.432829 |
| DNA | SC-cervical | 0.0376 | 1.143863 | 0.061085 | 0.061535 | 0.07573 | 0.05885 |
| DNA | SC-Thoracic | 0.02692 | 0.933734 | 0.025955 | 0.05011 | 0.064915 | 0.0355 |
| DNA | SC-Lumbar | 0.03615 | 0.992728 | 0.019125 | 0.034175 | 0.085165 | 0.051475 |
| DNA | DRG- | 0.0765 | 0.14319 | 0.08196 | 0.13722 | 0.04115 | 0.071625 |
| | cervical | | | | | | |
| DNA | DRG- | 0.165865 | 0.172363 | 0.07202 | 0.133455 | 0.04444 | 0.03139 |
| | Thoracic | | | | | | |
| DNA | DRG- | 0.218725 | 0.385712 | 0.146115 | 0.153205 | 0.032875 | 0.12034 |
| | Lumbar | | | | | | |
| DNA | Lung | 1.085639916 | 3.72 | 0.958576278 | 0.700015423 | 1.22442329 | 0.919823152 |
| DNA | Pancreas | 0.256670617 | 20.535 | 0.320558325 | 0.240633195 | 0.067860607 | 0.004802583 |
| DNA | Colon | 0.053867646 | 3.405 | 1.179065405 | 0.348969617 | 0.116867365 | 0.015288464 |
| DNA | Kidney | 0.896656371 | 26.635 | 4.861362029 | 0.532746958 | 0.386522209 | 7.973793288 |
| DNA | Liver | 207.332334 | 217.64 | 111.910319 | 193.8349405 | 448.5980021 | 213.0317219 |
| DNA | Adrenal | 1.647725996 | 0.69 | 1.561129869 | 1.871878 | 1.269473156 | 0.847293047 |
| DNA | Spleen | 14.93815481 | 20.43565 | 51.70294001 | 22.79095714 | 6.514778227 | 45.91987284 |
| DNA | Heart | 2.012377817 | 14.49 | 0.757528914 | 1.780956673 | 3.814571986 | 0.44694144 |
| DNA | Quadriceps | 0.724278943 | 1.285 | 0.476250457 | 1.366015493 | 5.611203726 | 0.646197937 |
| DNA | Diaphragm | — | 1.06 | — | — | — | — |

When calculated as fold over AAV9 the data were as shown in Table 22 below.

TABLE 22
Characterization of exemplary capsid variants

| Measure | Tissue | AAV9 | TTD-001 | TTD-002 | TTD-003 | TTD-004 | TTD-005 |
|---|---|---|---|---|---|---|---|
| mRNA | Frontal Cortex | 1.0 | 8378 | 2.4 | 19.3 | 21.8 | 6.8 |
| mRNA | Sensory Cortex | 1.0 | 2288 | 2.8 | 4.6 | 7.4 | 2.8 |
| mRNA | Motor Cortex | 1.0 | 14227 | 1.7 | 15.7 | 16.6 | 4.6 |
| mRNA | Putamen | 1.0 | 5805 | 2.3 | 19.3 | 18.7 | 1.9 |
| mRNA | Thalamus | 1.0 | 1097 | 5.3 | 38.8 | 29.0 | 4.2 |
| mRNA | Cerebellar | 1.0 | 1007 | 6.4 | 4.8 | 9.7 | 3.3 |
| | Cortex | | | | | | |
| mRNA | Dentate | 1.0 | 706 | — | — | — | — |
| | Nucleus | | | | | | |
| mRNA | Caudate | 1.0 | 3698 | — | — | — | — |
| mRNA | Hippocampus | 1.0 | 1305 | — | — | — | — |
| mRNA | SC-cervical | 1.0 | 973 | 1.8 | 2.2 | 7.2 | 0.5 |
| mRNA | SC-Thoracic | 1.0 | 61 | 0.1 | 0.7 | 0.4 | 0.1 |
| mRNA | SC-Lumbar | 1.0 | 267 | 1.9 | 0.5 | 1.6 | 0.9 |
| mRNA | DRG-cervical | 1.0 | 12 | 0.0 | 3.3 | 0.4 | 0.0 |
| mRNA | DRG-Thoracic | 1.0 | 9 | 0.0 | 15.7 | 0.2 | — |
| mRNA | DRG-Lumbar | 1.0 | 16 | 0.0 | 2.1 | 0.4 | 0.1 |
| mRNA | Lung | 1.0 | 0.11 | — | — | 2.9 | — |
| mRNA | Pancreas | — | — | — | — | — | — |
| mRNA | Colon | 1.0 | 0.28 | — | — | 10.2 | — |
| mRNA | Kidney | 1.0 | 0.15 | — | — | 0.9 | — |
| mRNA | Liver | 1.0 | 0.38 | — | — | 3.8 | — |
| mRNA | Adrenal | — | — | — | — | — | — |
| mRNA | Spleen | 1.0 | 0.07 | — | — | 1.0 | — |
| mRNA | Heart | 1.0 | 0.10 | — | — | 4.2 | — |
| mRNA | Quadriceps | 1.0 | — | — | — | 38.8 | — |
| mRNA | Diaphragm | 1.0 | — | — | — | 7.7 | — |
| DNA | Frontal Cortex | 1.0 | 27.29 | 1.3 | 0.9 | 4.9 | 1.6 |
| DNA | Sensory Cortex | 1.0 | 28.82 | 0.8 | 0.4 | 2.9 | 1.4 |
| DNA | Motor Cortex | 1.0 | 49.38 | 1.0 | 1.1 | 3.6 | 1.3 |
| DNA | Putamen | 1.0 | 52.96 | 2.1 | 2.6 | 4.7 | 1.2 |
| DNA | Thalamus | 1.0 | 39.63 | 1.3 | 1.7 | 4.1 | 1.7 |
| DNA | Cerebellar | 1.0 | 13.09 | 0.7 | 0.7 | 2.5 | 0.9 |
| | Cortex | | | | | | |
| DNA | Dentate | 1.0 | 7.44 | 8.4 | 1.7 | 4.2 | 2.2 |
| | Nucleus | | | | | | |
| DNA | Caudate | 1.0 | 49.73 | — | 0.7 | 3.1 | — |
| DNA | Hippocampus | 1.0 | 18.45 | 2.2 | 3.9 | 14.0 | 4.5 |
| DNA | SC-cervical | 1.0 | 30.42 | 1.6 | 1.6 | 2.0 | 1.6 |
| DNA | SC-Thoracic | 1.0 | 34.69 | 1.0 | 1.9 | 2.4 | 1.3 |
| DNA | SC-Lumbar | 1.0 | 27.46 | 0.5 | 0.9 | 2.4 | 1.4 |
| DNA | DRG-cervical | 1.0 | 1.87 | 1.1 | 1.8 | 0.5 | 0.9 |
| DNA | DRG-Thoracic | 1.0 | 1.04 | 0.4 | 0.8 | 0.3 | 0.2 |
| DNA | DRG-Lumbar | 1.0 | 1.76 | 0.7 | 0.7 | 0.2 | 0.6 |
| DNA | Lung | 1.0 | 3.43 | 0.9 | 0.6 | 1.1 | 0.8 |
| DNA | Pancreas | 1.0 | 80.01 | 1.2 | 0.9 | 0.3 | 0.0 |
| DNA | Colon | 1.0 | 63.21 | 21.9 | 6.5 | 2.2 | 0.3 |
| DNA | Kidney | 1.0 | 29.70 | 5.4 | 0.6 | 0.4 | 8.9 |
| DNA | Liver | 1.0 | 1.05 | 0.5 | 0.9 | 2.2 | 1.0 |
| DNA | Adrenal | 1.0 | 0.42 | 0.9 | 1.1 | 0.8 | 0.5 |
| DNA | Spleen | 1.0 | 1.37 | 3.5 | 1.5 | 0.4 | 3.1 |
| DNA | Heart | 1.0 | 7.20 | 0.4 | 0.9 | 1.9 | 0.2 |
| DNA | Quadriceps | 1.0 | 1.77 | 0.7 | 1.9 | 7.7 | 0.9 |
| DNA | Diaphragm | — | — | — | — | — | — |

Capsid variant TTD-001 showed greater than 5,000 fold increase in payload-HA levels delivered to the brain as compared to AAV9 and measured by qRT-PCR and normalized to TBP. In all CNS tissues measured, TTD-001 showed dramatically enhanced delivery of payload-HA as compared to AAV9. For example, analysis of the cervical, thoracic, and lumbar spinal cord revealed robust expression of payload-HA mRNA and DNA with the TTD-001 capsid relative to the wild-type AAV9 capsid (approximately 100-1000-fold higher mRNA expression and approximately 30-fold higher DNA levels (VG/cell) with TTD-001 relative to wild-type AAV9) (Tables 21 and 22).

Immunohistochemistry of fixed brain tissues revealed dramatic transduction in both NHP tested by TTD-001 of the dentate nucleus, cerebellar cortex, cerebral cortex, brain stem, hippocampus, thalamus and putamen. AAV9 transduction of the dentate nucleus, cerebellar cortex, cerebral cortex, hippocampus, thalamus and putamen appeared negligible in comparison. TTD-001 therefore demonstrated broad and robust expression and distribution in the brain following intravenous administration in NHPs. In the dorsal root ganglia, both TTD-001 and AAV9 showed similar IHC patterns. Additionally, robust expression of the payload-HA was observed in the Dorsal horn, Clarke's column, and ventral horn of the spinal cord with the TTD-001 capsid, whereas expression was relatively undetectable with the wild-type AAV9 capsid (FIG. 11).

### Example 4. Maturation of TTD-001 Capsid in NHPs

This Example describes maturation of the TTD-001 (SEQ ID NO: 3623 (DNA) and 3636 (amino acid), comprising SEQ ID NO: 3648) capsid variant to further enhance its transduction and biodistribution in the central nervous system and evolve the AAV capsid variants further. Two approaches were used to mature the TTD-001 capsid sequence in order to randomize and mutate within and around the peptide insert comprised within loop VIII of the capsid variant. In the first maturation approach, sets of three contiguous amino acids were randomized across the mutagenesis region in the TTD-001 sequence, which spanned from position 587 to position 602, numbered according to SEQ ID NO: 3636. In the second maturation approach, mutagenic primers were used to introduce point mutations at a low frequency, scattered across the mutagenesis region in the TTD-001 sequences ranging from position 587 to position 602, numbered according to SEQ ID NO: 3636. AAV capsid variants arising from each maturation approach for TTD-001 were pooled together, for subsequent testing and characterization in NHPs (Macaca fascicularis and Callithrix jacchus).

The library of pooled matured AAV capsid variants generated from TTD-001 matured AAV capsid variant were injected into two cynomolgus macaques (Macaca fascicularis), two marmosets (Callithrix jacchus). After a period in life, the brains of the NHPs were isolated and RNA was extracted from three samples per NHP. Following RNA recovery and RT-PCR amplification, a systematic NGS enrichment analysis was performed to calculate the fold enrichment ratio relative to the corresponding TTD-001 control, and the peptides comprised within the variants were identified. The coefficient of variance (CV) was calculated for each peptide across the six samples, and those that had a CV value <1 were identified, as these were the peptides that were reliably detected in 5/6 or 6/6 of the brain samples isolated from the two NHPs. The average number of reads for each peptide across the samples investigated was also quantified. These TTD-001 matured capsid variants and their peptide sequences are provided in Table 23 (cynomolgus macaques (Macaca fascicularis)) and Table 24 (marmosets (Callithrix jacchus)).

As shown in Table 23, approximately 338 TTD-001 matured capsid variants demonstrated increased expression relative to the non-matured TTD-001 control, and several variants demonstrated greater than a two-fold enrichment relative to the non-matured TTD-001 control, in cynomolgus macaques (Macaca fascicularis). Also, across the peptides comprised within the TTD-001 matured capsid variants with the greatest fold-enrichment relative to the non-matured TTD-001 capsid in the brains of cynomolgus macaques, it was observed that the modifications in the variant sequences appeared in the C-terminal portion, specifically at residues corresponding to positions 593-595 of a reference sequence numbered according to the amino acid sequence of SEQ ID NO: 138. Additionally, 378 of the top peptides in Table 23 had an average read value of 1 or greater per sample, demonstrating that more functional capsid material was recovered, which could be indicative of less aggregation.

As shown in Table 24, many TTD-001 matured capsid variants demonstrated increased expression relative to both the AAV9 and the non-matured TTD-001 controls in the brains of marmosets. Approximately, 967 TTD-001 matured variants demonstrated increased expression relative to the non-matured TTD-001 control in the brain of marmosets, with 296 variants showing at least a 10-fold enrichment or greater relative to the non-matured TTD-001 control. Approximately, 850 TTD-001 matured variants demonstrated increased expression relative to AAV9 in the brain of marmosets, with 222 variants showing at least a 10-fold enrichment or greater relative to AAV9. With respect to those TTD-001 matured variants that demonstrated an increased expression in marmosets, it was observed that the majority comprised an amino acid other than Q at position 604 (e.g., Q604) numbered according to SEQ ID NO: 5, 8, or 3636 or at position 597 (Q597) numbered according to SEQ ID NO: 138 (e.g., an E, H, K, or P), such that they comprised the triplet “VEN,” “VHN,” “VKN,” or “VPN” at their C-terminus (corresponding to positions 596-598 of SEQ ID: 138 or positions 603-605 of SEQ ID NO: 5, 8, and 3636). Many of these TTD-001 matured variants also demonstrated an increased expression in the brain of cynomolgus macaques relative to AAV9 (Table 24), including the TTD-001 matured capsid variant comprising the sequence PLNGAVHLYAQAQLSPVKN (SEQ ID NO: 566) and the TTD-001 matured capsid variant comprising the sequence PLNGAVHLYAQAQTGWVPN (SEQ ID NO: 314).

The fold-change in expression relative to AAV9 and TTD-001 was also calculated in the DRG, heart, muscle (quadriceps), and liver for the TTD-001 matured variants in cynomolgus macaques. The fold-change in the DRG is shown in Table 24, with several variants showing decreased or comparable expression in the DRG relative to AAV9. These variants also demonstrated comparable or lower expression relative to AAV9 in the heart, muscle, and liver.

Taken together, these data demonstrate that following two maturation approaches, matured TTD-001 capsid variants with loop VIII modifications were generated with significantly enhanced CNS tropism in NHPs (cynomolgus macaques (Macaca fascicularis) and marmosets (Callithrix jacchus)), compared to the corresponding non-matured TTD-001 capsid variant, which already exhibited a significant fold enrichment over AAV9 in the NHP brain.

TABLE 23
NGS fold-enrichment of TTD-001 matured AAV
capsid variants in the brain of NHPs

| | | | Fold | |
|---|---|---|---|---|
| | SEQ | | enrichment | |
| Peptide | ID | | over | Avg. |
| Sequence | NO: | CV | TTD-001 | Reads |
| QLNGAVHLYA | 139 | 0.206 | 5.617 | 0.09 |
| QAQLSPVQN | | | | |
| PLDGAVHLYA | 140 | 0.829 | 5.133 | 4.02 |
| QPQTGWVQN | | | | |
| QLNGAVHLYA | 141 | 0.770 | 4.088 | 0.09 |
| QAQTMSVQN | | | | |
| PLDSSVHLYA | 142 | 0.882 | 3.891 | 1.4 |
| QAQTGWVQN | | | | |
| PLNGAVHLYA | 143 | 0.879 | 3.879 | 5.47 |
| QAQTTKVQN | | | | |
| PLDGAVHLYA | 144 | 0.586 | 3.835 | 11.08 |
| QAQTGSVQN | | | | |
| ALNGAVHLYA | 145 | 0.865 | 3.765 | 0.09 |
| QAQTTSVQN | | | | |
| PLNGSVHLYA | 146 | 0.509 | 3.708 | 0.09 |
| QAQTMSVQN | | | | |
| QLNGAVHLYA | 147 | 0.310 | 3.638 | 0.14 |
| QAQTSPVQN | | | | |
| PLNGAVHLYA | 148 | 0.547 | 3.634 | 5.61 |
| QAQTMKVQN | | | | |
| PLNGAVHLYA | 149 | 0.397 | 3.550 | 21.87 |
| QAQVAQVQN | | | | |
| PLDGAVHLYA | 150 | 0.771 | 3.509 | 2.29 |
| QAQTGGVQN | | | | |
| PLNGAVHLYA | 151 | 0.344 | 3.373 | 0.09 |
| QAQTAWDQN | | | | |
| PLNGSVHLYA | 152 | 0.190 | 3.335 | 0.23 |
| QAQTGWDQN | | | | |
| PLNGAVHLYA | 153 | 0.235 | 3.287 | 0.14 |
| QAQTGSVQH | | | | |
| PLNGAVHLYA | 154 | 0.564 | 3.259 | 4.25 |
| QAQVKQVQN | | | | |
| PLNGAVHLYA | 155 | 0.763 | 3.239 | 24.54 |
| QAQSAPVQN | | | | |
| PLNGAVHLYA | 156 | 0.591 | 3.156 | 6.73 |
| QAQLSKVQN | | | | |
| PLNGAVHLYA | 157 | 0.587 | 3.107 | 31.03 |
| QAQLAPVQN | | | | |
| PLNGAVHLYA | 158 | 0.416 | 3.061 | 15.33 |
| QAQLAQVQN | | | | |
| QLNGAVHLYA | 159 | 0.295 | 3.051 | 0.09 |
| QAQVASVQN | | | | |
| PLNGAVHLYA | 160 | 0.877 | 2.998 | 8.97 |
| QAQTAKVQN | | | | |
| PLNGAVHLYA | 161 | 0.678 | 2.971 | 3.27 |
| QAQSAKVQN | | | | |
| PLNGAVHLYA | 162 | 0.095 | 2.962 | 0.09 |
| QAQTGCFQN | | | | |
| PLNGAVHLYA | 163 | 0.705 | 2.958 | 5.51 |
| QAQTQKVQN | | | | |
| PLNGSVHLYA | 164 | 0.529 | 2.873 | 0.09 |
| QAQTTSVQN | | | | |
| PLNGGVHLYA | 165 | 0.820 | 2.858 | 4.25 |
| QAQTGRVQN | | | | |
| PLNGAVHLYA | 166 | 0.410 | 2.854 | 30.94 |
| QAQTVAVQN | | | | |
| ALNGAVHLYA | 167 | 0.542 | 2.851 | 0.09 |
| QAQSSPVQN | | | | |
| PLNGAVHLYA | 168 | 0.313 | 2.820 | 63.28 |
| QAQLSPVQN | | | | |
| QLNGAVHLYA | 169 | 0.846 | 2.769 | 0.14 |
| QAQTTSVQN | | | | |
| PLNGAVHLYA | 170 | 0.334 | 2.719 | 18.37 |
| QAQTTQVQN | | | | |
| PLNGAVHLYA | 171 | 0.523 | 2.707 | 25.84 |
| QAQTAQVQN | | | | |
| QLNGAVHLYA | 172 | 0.195 | 2.682 | 0.09 |
| QAQTVAVQN | | | | |
| PLNGAVHLYA | 173 | 0.682 | 2.652 | 3.27 |
| QAQRIAVQN | | | | |
| PLNGAVHLYA | 174 | 0.340 | 2.634 | 17.34 |
| QAQRASVQN | | | | |
| PLNGAVHLYA | 175 | 0.400 | 2.620 | 45.19 |
| QAQTTPVQN | | | | |
| QLNGAVHLYA | 176 | 0.474 | 2.613 | 0.09 |
| QAQLASVQN | | | | |
| PLNGAVHLYA | 177 | 0.528 | 2.574 | 39.86 |
| QAQLTPVQN | | | | |
| PLNGAVHLYA | 178 | 0.519 | 2.572 | 26.5 |
| QAQSTPVQN | | | | |
| PLNGAVHLYA | 179 | 0.335 | 2.562 | 72.81 |
| QAQTSPVQN | | | | |
| QLNGAVHLYA | 180 | 0.428 | 2.528 | 0.09 |
| QAQVSQVQN | | | | |
| PLNGAVHLYA | 181 | 0.625 | 2.523 | 19.96 |
| QAQTMQVQN | | | | |
| PLNGAVHLYA | 182 | 0.458 | 2.476 | 8.32 |
| QAQTSKVQN | | | | |
| SLNGAVHLYA | 183 | 0.282 | 2.417 | 0.09 |
| QAQVSPVQN | | | | |
| PLNGAVHLYA | 184 | 0.259 | 2.411 | 34.72 |
| QAQVSQVQN | | | | |
| PLNGAVHLYA | 185 | 0.721 | 2.404 | 63.42 |
| QAQVSPVQN | | | | |
| PLNGAVHLYA | 186 | 0.586 | 2.389 | 15.56 |
| QAQTVQVQN | | | | |
| PLNGAVHLYA | 187 | 0.476 | 2.367 | 13.65 |
| QAQVTAVQN | | | | |
| PLNGAVHLYA | 188 | 0.715 | 2.353 | 8.74 |
| QAQRQPVQN | | | | |
| PLNGNVHLYA | 189 | 0.732 | 2.346 | 2.38 |
| QAQTGGVQN | | | | |
| PLDGAVHLYA | 190 | 0.697 | 2.339 | 7.06 |
| QAQTAWVQN | | | | |
| PLNGAVHLYA | 191 | 0.785 | 2.331 | 9.91 |
| QAQISGVQN | | | | |
| PLNGAVHLYA | 192 | 0.794 | 2.324 | 6.22 |
| QAQVRPVQN | | | | |
| PLNGAVHLYA | 193 | 0.722 | 2.299 | 20.47 |
| QAQLGPVQN | | | | |
| PLNGAVHLYA | 194 | 0.404 | 2.290 | 4.91 |
| QAQTNQVQN | | | | |
| PLNGAVHLYA | 195 | 0.291 | 2.240 | 14.39 |
| QAQVQQVQN | | | | |
| PLNGAVHLYA | 196 | 0.520 | 2.238 | 9.21 |
| QAQVANVQN | | | | |
| PLNGAVHLYA | 197 | 0.664 | 2.200 | 10.75 |
| QAQAAPVQN | | | | |
| QLNGAVHLYA | 198 | 0.388 | 2.199 | 0.09 |
| QAQVAQVQN | | | | |
| PLNGAVHLYA | 199 | 0.487 | 2.195 | 21.59 |
| QAQRSTVQN | | | | |
| PLNGAVHLYA | 200 | 0.334 | 2.190 | 29.72 |
| QAQTMAVQN | | | | |
| PLNGAVHLYA | 201 | 0.838 | 2.182 | 7.1 |
| QAQIQPVQN | | | | |
| PLNGAVHLYA | 202 | 0.567 | 2.181 | 15.52 |
| QAQIASVQN | | | | |
| PLNGAVHLYA | 203 | 0.172 | 2.176 | 76.22 |
| QAQTVSVQN | | | | |
| PLNGAVHLYA | 204 | 0.585 | 2.156 | 12.43 |
| QAQRGSVQN | | | | |
| PLNGAVHLYA | 205 | 0.376 | 2.143 | 12.85 |
| QAQNSPVQN | | | | |
| PLNGAVHLYA | 206 | 0.363 | 2.131 | 24.3 |
| QAQLQPVQN | | | | |
| PLNGAVHLYA | 207 | 0.550 | 2.125 | 11.68 |
| QAQVTGVQN | | | | |
| PLNGAVHLYA | 208 | 0.810 | 2.121 | 11.26 |
| QAQVMQVQN | | | | |
| PLNGAVHLYA | 209 | 0.697 | 2.104 | 14.86 |
| QAQSMAVQN | | | | |
| PLNGAVHLYA | 210 | 0.611 | 2.104 | 4.49 |
| QAQVGKVQN | | | | |
| ALNGAVHLYA | 211 | 0.652 | 2.101 | 0.09 |
| QAQSAPVQN | | | | |
| PLNGAVHLYA | 212 | 0.498 | 2.101 | 3.97 |
| QAQIQSVQN | | | | |
| PLNGAVHLYA | 213 | 0.537 | 2.097 | 17.15 |
| QAQCSPVQN | | | | |
| PLNGAVHLYA | 214 | 0.821 | 2.079 | 3.97 |
| QAQLQRVQN | | | | |
| PLNGAVHLYA | 215 | 0.330 | 2.063 | 0.09 |
| QAQTAWVQH | | | | |
| PLDGAVHLYA | 216 | 0.650 | 2.062 | 0.09 |
| QAQTGWDQN | | | | |
| PLNGAVHLYA | 217 | 0.411 | 2.062 | 0.89 |
| QAQTPPVQN | | | | |
| PLNGAVHLYA | 218 | 0.635 | 2.054 | 1.68 |
| QAQVTKVQN | | | | |
| PLNGAVHLYA | 219 | 0.556 | 2.033 | 50.94 |
| QAQSSPVQN | | | | |
| PLNGAVHLYA | 220 | 0.793 | 2.015 | 9.07 |
| QAQAGPVQN | | | | |
| PLNGAVHLYA | 221 | 0.773 | 2.014 | 5.89 |
| QAQLARVQN | | | | |
| PLNGAVHLYA | 222 | 0.789 | 2.013 | 16.36 |
| QAQTTTVQN | | | | |
| PLNGAVHLYA | 223 | 0.254 | 2.011 | 0.09 |
| QAQTGGFQN | | | | |
| PLNGAVHLYA | 224 | 0.657 | 2.010 | 24.44 |
| QAQTLQVQN | | | | |
| PLNGAVHLYA | 225 | 0.359 | 2.005 | 63.42 |
| QAQTMSVQN | | | | |
| QLNGAVHLYA | 226 | 0.675 | 2.004 | 0.09 |
| QAQLQPVQN | | | | |
| PLNGAVHLYA | 227 | 0.894 | 1.994 | 10.47 |
| QAQVAKVQN | | | | |
| PLNGAVHLYA | 228 | 0.217 | 1.989 | 7.24 |
| QAQRAAVQN | | | | |
| PLNGAVHLYA | 229 | 0.413 | 1.988 | 38.79 |
| QAQTVGVQN | | | | |
| PLNGAVHLYA | 230 | 0.381 | 1.972 | 15.84 |
| QAQLNPVQN | | | | |
| PLNGAVHLYA | 231 | 0.379 | 1.971 | 26.17 |
| QAQLSQVQN | | | | |
| PLNGTVHLYA | 232 | 0.421 | 1.967 | 23.37 |
| QAQTGSVQN | | | | |
| PLNGAVHLYA | 233 | 0.543 | 1.960 | 12.95 |
| QAQTKPVQN | | | | |
| PLNGAVHLYA | 234 | 0.603 | 1.957 | 7.71 |
| QAQTNAVQN | | | | |
| ALDGAVHLYA | 235 | 0.660 | 1.955 | 21.4 |
| QAQTGWVQN | | | | |
| PLNGAVHLYA | 236 | 0.303 | 1.955 | 22.06 |
| QAQLATVQN | | | | |
| PLNGAVHLYA | 237 | 0.794 | 1.947 | 40.85 |
| QAQVTPVQN | | | | |
| PLNGAVHLYA | 238 | 0.339 | 1.946 | 22.2 |
| QAQVQAVQN | | | | |
| PLNGAVHLYA | 239 | 0.645 | 1.926 | 58.32 |
| QAQTTSVQN | | | | |
| PLNGAVHLYA | 240 | 0.702 | 1.923 | 9.21 |
| QAQCTPVQN | | | | |
| ALNGAVHLYA | 241 | 0.621 | 1.916 | 9.58 |
| QAQTGRVQN | | | | |
| PLNGSVHLYA | 242 | 0.173 | 1.908 | 0.09 |
| QAQTGWVQD | | | | |
| PLNGAVHLYA | 243 | 0.555 | 1.903 | 30.47 |
| QAQTAGVQN | | | | |
| PLNGAVHLYA | 244 | 0.431 | 1.902 | 29.82 |
| QAQTSQVQN | | | | |
| PLNGAVHLYA | 245 | 0.443 | 1.895 | 6.4 |
| QAQTMNVQN | | | | |
| PLNGAVHLYA | 246 | 0.323 | 1.885 | 34.16 |
| QAQTSTVQN | | | | |
| PLNGAVHLYA | 247 | 0.712 | 1.882 | 12.43 |
| QAQVKPVQN | | | | |
| PLNGAVHLYA | 248 | 0.627 | 1.878 | 24.91 |
| QAQASPVQN | | | | |
| PLNGAVHLYA | 249 | 0.447 | 1.873 | 23.27 |
| QAQVAAVQN | | | | |
| PLNGAVHLYA | 250 | 0.692 | 1.869 | 8.74 |
| QAQLKSVQN | | | | |
| PLNGAVHLYA | 251 | 0.834 | 1.855 | 8.09 |
| QAQIAAVQN | | | | |
| PLNGAVHLYA | 252 | 0.115 | 1.850 | 26.78 |
| QAQTAAVQN | | | | |
| PLNGAVHLYA | 253 | 0.742 | 1.850 | 10.05 |
| QAQTKAVQN | | | | |
| PLNGAVHLYA | 254 | 0.223 | 1.848 | 0.09 |
| QAQTGSVQS | | | | |
| PLNGAVHLYA | 255 | 0.518 | 1.845 | 7.66 |
| QAQVSNVQN | | | | |
| PLNGAVHLYA | 256 | 0.690 | 1.834 | 38.65 |
| QAQTAPVQN | | | | |
| PLNGAVHLYA | 257 | 0.740 | 1.833 | 28.13 |
| QAQLMPVQN | | | | |
| PLNGAVHLYA | 258 | 0.798 | 1.824 | 8.88 |
| QAQLHPVQN | | | | |
| PLNGAVHLYA | 259 | 0.793 | 1.823 | 5.09 |
| QAQRAQVQN | | | | |
| PLNGAVHLYA | 260 | 0.871 | 1.823 | 1.92 |
| QAQLTNVQN | | | | |
| PLNGAVHLYA | 261 | 0.766 | 1.822 | 13.18 |
| QAQRTTVQN | | | | |
| PLNGAVHLYA | 262 | 0.700 | 1.821 | 17.9 |
| QAQTSVVQN | | | | |
| SLNGAVHLYA | 263 | 0.867 | 1.819 | 0.14 |
| QAQTMSVQN | | | | |
| ALNGAVHLYA | 264 | 0.672 | 1.818 | 0.09 |
| QAQTLAVQN | | | | |
| PLNGAVHLYA | 265 | 0.736 | 1.816 | 12.2 |
| QAQRMSVQN | | | | |
| ALNGAVHLYA | 266 | 0.342 | 1.813 | 0.14 |
| QAQLTPVQN | | | | |
| PLNGAVHLYA | 267 | 0.785 | 1.807 | 3.65 |
| QAQVGNVQN | | | | |
| PLNGAVHLYA | 268 | 0.615 | 1.805 | 4.91 |
| QAQLMQVQN | | | | |
| PLNGSVHLYA | 269 | 0.550 | 1.795 | 0.09 |
| QAQTAQVQN | | | | |
| PLNGAVHLYA | 270 | 0.544 | 1.793 | 24.12 |
| QAQTATVQN | | | | |
| PLNGAVHLYA | 271 | 0.727 | 1.790 | 7.8 |
| QAQVHPVQN | | | | |
| PLNGAVHLYA | 272 | 0.836 | 1.771 | 0.09 |
| QAQVSPVQT | | | | |
| PLNGAVHLYA | 273 | 0.638 | 1.766 | 9.11 |
| QAQISSVQN | | | | |
| PLNGAVHLYA | 274 | 0.333 | 1.765 | 62.16 |
| QAQVASVQN | | | | |
| PLNGAVHLYA | 275 | 0.347 | 1.757 | 0.09 |
| QAQTRWDQN | | | | |
| PLNGAVHLYA | 276 | 0.508 | 1.750 | 8.88 |
| QAQTMTVQN | | | | |
| PLNGAVHLYA | 277 | 0.551 | 1.747 | 34.21 |
| QAQRSSVQN | | | | |
| PLNGGVHLYA | 278 | 0.547 | 1.743 | 0.09 |
| QAQTGWVRN | | | | |
| PLNGAVHLYA | 279 | 0.281 | 1.741 | 213 |
| QAQTAWVQN | | | | |
| PLNGSVHLYA | 280 | 0.130 | 1.734 | 0.28 |
| QAQPGWVQN | | | | |
| ALNGAVHLYA | 281 | 0.238 | 1.724 | 0.09 |
| QAQTGWAQN | | | | |
| PLNGAVHLYA | 282 | 0.791 | 1.724 | 8.93 |
| QAQRTGVQN | | | | |
| ALNGAVHLYA | 283 | 0.370 | 1.720 | 0.09 |
| QAQTGWVQS | | | | |
| PLNGAVHLYA | 284 | 0.561 | 1.701 | 22.34 |
| QAQVATVQN | | | | |
| PLNGAVHLYA | 285 | 0.480 | 1.700 | 16.68 |
| QAQVTSVQN | | | | |
| PLNGAVHLYA | 286 | 0.313 | 1.699 | 74.78 |
| QAQVSSVQN | | | | |
| PLNGSVHLYA | 287 | 0.353 | 1.683 | 0.14 |
| QAQTSSVQN | | | | |
| PLNGAVHLYA | 288 | 0.348 | 1.682 | 18.83 |
| QAQTNSVQN | | | | |
| PLNGAVHLYA | 289 | 0.815 | 1.681 | 6.03 |
| QAQVKAVQN | | | | |
| PLNGAVHLYA | 290 | 0.689 | 1.666 | 19.02 |
| QAQSGPVQN | | | | |
| PLNGAVHLYA | 291 | 0.660 | 1.663 | 25.61 |
| QAQTGPVQN | | | | |
| PLNGAVHLYA | 292 | 0.537 | 1.660 | 22.9 |
| QAQTAMVQN | | | | |
| PLNGAVHLYA | 293 | 0.358 | 1.628 | 32.11 |
| QAQTQPVQN | | | | |
| PLNGSVHLYA | 294 | 0.533 | 1.625 | 32.76 |
| QAQTGSVQN | | | | |
| PLNGAVHLYA | 295 | 0.519 | 1.625 | 14.44 |
| QAQTQQVQN | | | | |
| PLNGAVHLYA | 296 | 0.824 | 1.620 | 9.86 |
| QAQVSRVQN | | | | |
| QLNGAVHLYA | 297 | 0.436 | 1.617 | 0.09 |
| QAQVLPVQN | | | | |
| PLNGAVHLYA | 298 | 0.266 | 1.610 | 0.14 |
| QAQTGWVQP | | | | |
| QLNGAVHLYA | 299 | 0.776 | 1.609 | 0.09 |
| QAQLGSVQN | | | | |
| PLNGAVHLYA | 300 | 0.360 | 1.604 | 40.29 |
| QAQVSAVQN | | | | |
| PLNGAVHLYA | 301 | 0.593 | 1.604 | 7.8 |
| QAQVLSVQN | | | | |
| PLNGAVHLYA | 302 | 0.861 | 1.601 | 5.05 |
| QAQTQHVQN | | | | |
| PLNGAVHLYA | 303 | 0.113 | 1.590 | 52.72 |
| QAQLASVQN | | | | |
| PLNGAVHLYA | 304 | 0.649 | 1.581 | 8.83 |
| QAQQAPVQN | | | | |
| PLNGAVHLYA | 305 | 0.577 | 1.574 | 3.08 |
| QAQNAQVQN | | | | |
| PLNGAVHLYA | 306 | 0.760 | 1.567 | 12.81 |
| QAQATPVQN | | | | |
| PLNGAVHLYA | 307 | 0.544 | 1.567 | 28.13 |
| QAQVQPVQN | | | | |
| PLNGAVHLYA | 308 | 0.537 | 1.563 | 33.13 |
| QAQTTAVQN | | | | |
| PLNGAVHLYA | 309 | 0.455 | 1.561 | 3.46 |
| QAQTGWVRN | | | | |
| PLNGAVHLYA | 310 | 0.357 | 1.561 | 21.83 |
| QAQLAAVQN | | | | |
| PLNGAVHLYA | 311 | 0.418 | 1.555 | 0.09 |
| QAQTSPDQN | | | | |
| PLNGAVHLYA | 312 | 0.393 | 1.553 | 16.36 |
| QAQRSGVQN | | | | |
| PLNGAVHLYA | 313 | 0.218 | 1.551 | 0.65 |
| QAQTGGVQT | | | | |
| PLNGAVHLYA | 314 | 0.274 | 1.546 | 0.93 |
| QAQTGWVPN | | | | |
| PLDSAVHLYA | 315 | 0.833 | 1.539 | 10.89 |
| QAQTGWVQN | | | | |
| PLNGAVHLYA | 316 | 0.496 | 1.537 | 0.09 |
| QAQTTPVQT | | | | |
| PLNGAVHLYA | 317 | 0.540 | 1.535 | 7.94 |
| QAQLMAVQN | | | | |
| PLNGAVHLYA | 318 | 0.596 | 1.532 | 36.69 |
| QAQTMPVQN | | | | |
| PLNGAVHHYA | 319 | 0.391 | 1.529 | 0.09 |
| QAQTSPVQN | | | | |
| PLNGAVHLYA | 320 | 0.756 | 1.527 | 7.8 |
| QAQLANVQN | | | | |
| PLNGAVHLYA | 321 | 0.606 | 1.520 | 11.68 |
| QAQVSTVQN | | | | |
| PLNGAVHLYA | 322 | 0.602 | 1.518 | 12.9 |
| QAQSAQVQN | | | | |
| PLNGAVHLYA | 323 | 0.838 | 1.508 | 8.13 |
| QAQNTPVQN | | | | |
| PLNGAVHLYA | 324 | 0.381 | 1.495 | 0.09 |
| QAQVSSVQT | | | | |
| PLNGAVHLYA | 325 | 0.284 | 1.492 | 0.09 |
| QAQTVSVKN | | | | |
| PLNGAVHLYA | 326 | 0.710 | 1.488 | 9.21 |
| QPQTGLVQN | | | | |
| PLNGAVHLYA | 327 | 0.287 | 1.487 | 621.2 |
| QAQTGSVQN | | | | |
| SLNGAVHLYA | 328 | 0.437 | 1.485 | 0.09 |
| QAQTLPVQN | | | | |
| PLNGAVHLYA | 329 | 0.440 | 1.478 | 0.09 |
| QAQTGGAQN | | | | |
| SLNGAVHLYA | 330 | 0.618 | 1.477 | 0.09 |
| QAQTAQVQN | | | | |
| PLNGAVHLYA | 331 | 0.684 | 1.466 | 14.21 |
| QAQTAVVQN | | | | |
| PLNGAVHLYA | 332 | 0.533 | 1.456 | 17.71 |
| QAQRLGVQN | | | | |
| PLNGSVHLYA | 333 | 0.207 | 1.453 | 0.37 |
| QAQTGWVQH | | | | |
| PLNGAVHLYA | 334 | 0.489 | 1.452 | 21.26 |
| QAQRTLVQN | | | | |
| SLNGAVHLYA | 335 | 0.559 | 1.451 | 0.09 |
| QAQTMAVQN | | | | |
| PLNGAVHLYA | 336 | 0.707 | 1.447 | 10.23 |
| QAQTQMVQN | | | | |
| PLNGAVHLYA | 337 | 0.663 | 1.442 | 10.52 |
| QAQITPVQN | | | | |
| PLNGAVHLYA | 338 | 0.199 | 1.434 | 0.09 |
| QAQTVWVQK | | | | |
| PLNGAVHLYA | 339 | 0.584 | 1.423 | 13.97 |
| QAQRSAVQN | | | | |
| PLNGAVHLYA | 340 | 0.332 | 1.420 | 99.87 |
| QAQTASVQN | | | | |
| PLNGAVHLYA | 341 | 0.536 | 1.417 | 11.22 |
| QAQTMGVQN | | | | |
| PLNGAVHLYA | 342 | 0.256 | 1.414 | 0.28 |
| QAQTGGVQH | | | | |
| PLNGAVHLYA | 343 | 0.415 | 1.412 | 36.36 |
| QAQVQSVQN | | | | |
| PLNGAVHLYA | 344 | 0.324 | 1.411 | 500.1 |
| QAQTGGVQN | | | | |
| SLNGAVHLYA | 345 | 0.471 | 1.411 | 0.09 |
| QAQTAPVQN | | | | |
| PLNGAVHLYA | 346 | 0.680 | 1.410 | 16.68 |
| QAQISPVQN | | | | |
| PLNGAVHLYA | 347 | 0.345 | 1.409 | 0.09 |
| QPQTGWVKN | | | | |
| PLNGAVHLYA | 348 | 0.242 | 1.409 | 0.14 |
| QAQTGSAQN | | | | |
| PLNGAVHLYA | 349 | 0.474 | 1.393 | 2.62 |
| QAQTGWAQN | | | | |
| PLNGAVHLYA | 350 | 0.383 | 1.392 | 0.09 |
| QAQTMSV?T | | | | |
| PLNGSVHLYA | 351 | 0.649 | 1.391 | 12.29 |
| QAQTAWVQN | | | | |
| PLNGAVHLYA | 352 | 0.410 | 1.387 | 8.13 |
| QAQVGGVQN | | | | |
| PLNGAVHLYA | 353 | 0.496 | 1.387 | 0.09 |
| KAQTSPVQN | | | | |
| PLNGAVHLYA | 354 | 0.768 | 1.381 | 0.09 |
| QAQLAPVQT | | | | |
| PLDGAVHLYA | 355 | 0.901 | 1.375 | 0.09 |
| QAQTALVQN | | | | |
| PLNGAVHLYA | 356 | 0.594 | 1.368 | 58.98 |
| QAQTALVQN | | | | |
| PLNGAVHLYA | 357 | 0.470 | 1.366 | 19.63 |
| QAQLAGVQN | | | | |
| PLNGAVHLYA | 358 | 0.613 | 1.365 | 9.53 |
| QAQRTAVQN | | | | |
| PLNGAVHLYA | 359 | 0.540 | 1.363 | 22.15 |
| QAQRSPVQN | | | | |
| PLNGAVHLYA | 360 | 0.513 | 1.360 | 39.82 |
| QAQTLAVQN | | | | |
| PLNGAVHLYA | 361 | 0.322 | 1.351 | 5.42 |
| QAQLAHVQN | | | | |
| PLNGAVHLYA | 362 | 0.352 | 1.348 | 36.69 |
| QAQTSLVQN | | | | |
| PLNGAVHLYA | 363 | 0.548 | 1.347 | 30.75 |
| QAQRLSVQN | | | | |
| PLNGAVHLYD | 364 | 0.406 | 1.344 | 0.09 |
| QAQLSPVQN | | | | |
| PLNGAVHLYA | 365 | 0.424 | 1.339 | 13.97 |
| QAQLMGVQN | | | | |
| PLNGSVHLYA | 366 | 0.147 | 1.336 | 1.03 |
| QAQTGWVQT | | | | |
| PLNGAVHLYA | 367 | 0.588 | 1.335 | 11.26 |
| QAQSMQVQN | | | | |
| PLNGGVHLYA | 368 | 0.483 | 1.334 | 0.14 |
| QAQTGWFQN | | | | |
| PLNGAVHLYA | 369 | 0.834 | 1.326 | 14.02 |
| QAQTQTVQN | | | | |
| PLDGAVHLYS | 370 | 0.860 | 1.323 | 9.44 |
| QAQTGWVQN | | | | |
| PLNGAVHLYA | 371 | 0.325 | 1.319 | 0.14 |
| QAQTGWEQN | | | | |
| PLNGAVHLYA | 372 | 0.487 | 1.317 | 29.44 |
| QAQVGSVQN | | | | |
| PLNGAVHLYA | 373 | 0.339 | 1.317 | 24.86 |
| QAQVSGVQN | | | | |
| PLNGAVHLYA | 374 | 0.767 | 1.312 | 13.04 |
| QAQVMAVQN | | | | |
| PLNGAVHLYA | 375 | 0.814 | 1.309 | 23.37 |
| QAQIGGVQN | | | | |
| PLNGAVHLYA | 376 | 0.841 | 1.301 | 5.47 |
| QAQIAGVQN | | | | |
| PLNGAVHLYA | 377 | 0.200 | 1.297 | 0.09 |
| KAQTVSVQN | | | | |
| PLNGAVHLYS | 378 | 0.838 | 1.297 | 4.3 |
| QAQTGRVQN | | | | |
| PLNGAVHLYA | 379 | 0.826 | 1.296 | 7.34 |
| QAQLSHVQN | | | | |
| PLNGAVHLYA | 380 | 0.865 | 1.288 | 4.77 |
| QAQVQTVQN | | | | |
| SLDGAVHLYA | 381 | 0.875 | 1.287 | 61.08 |
| QAQTGWVQN | | | | |
| PLNGAVHLYA | 382 | 0.326 | 1.287 | 0.09 |
| QPQTGWDQN | | | | |
| PLNGAVHLYA | 383 | 0.564 | 1.284 | 11.36 |
| QAQRNSVQN | | | | |
| PLNGAVHLYA | 384 | 0.533 | 1.283 | 57.16 |
| QAQTLPVQN | | | | |
| PLNGAVHLYA | 385 | 0.569 | 1.282 | 5.09 |
| QAQTKQVQN | | | | |
| PLNGSVHLYA | 386 | 0.144 | 1.279 | 972 |
| QAQTGWVQN | | | | |
| PLNGAVHLYA | 387 | 0.593 | 1.278 | 11.17 |
| QAQLGQVQN | | | | |
| SLNGAVHLYA | 388 | 0.684 | 1.277 | 19.35 |
| QAQTGRVQN | | | | |
| PLDGAVHLYA | 389 | 0.647 | 1.275 | 263.1 |
| QAQTGWVQN | | | | |
| PLNGSVHLYA | 390 | 0.181 | 1.273 | 0.19 |
| QAQTGWAQN | | | | |
| TLNGAVHLYA | 391 | 0.405 | 1.266 | 0.09 |
| QAQVASVQN | | | | |
| PLNGAVHLYA | 392 | 0.733 | 1.261 | 2.85 |
| QAQNMQVQN | | | | |
| PLNGAVHLYA | 393 | 0.863 | 1.258 | 3.18 |
| QAQNVQVQN | | | | |
| PLNGAVHLYA | 394 | 0.299 | 1.250 | 0.14 |
| QAQTGSVQI | | | | |
| PLNGTVHLYA | 395 | 0.840 | 1.249 | 15.75 |
| QAQTGGVQN | | | | |
| PLNGAVHLYA | 396 | 0.805 | 1.248 | 0.09 |
| KAQVSPVQN | | | | |
| PLNGAVHLYA | 397 | 0.765 | 1.241 | 6.4 |
| QAQRGGVQN | | | | |
| PLNGGVHLYA | 398 | 0.335 | 1.240 | 25.94 |
| QAQTGLVQN | | | | |
| PLNGAVHLYA | 399 | 0.542 | 1.237 | 33.32 |
| QAQVMSVQN | | | | |
| PLNGAVHLYA | 400 | 0.401 | 1.235 | 15.05 |
| QAQTTGVQN | | | | |
| PLNGAVHLYA | 401 | 0.375 | 1.229 | 0.09 |
| QAQLSPVQK | | | | |
| SLNGAVHLYA | 402 | 0.677 | 1.226 | 0.09 |
| QAQTAAVQN | | | | |
| PINGAVHLYA | 403 | 0.351 | 1.223 | 0.14 |
| QAQTSPVQN | | | | |
| PLNGAVHLYA | 404 | 0.691 | 1.220 | 15.28 |
| QAQLSRVQN | | | | |
| PLNGAVHLYA | 405 | 0.259 | 1.213 | 0.09 |
| QAQVSSVQK | | | | |
| PLNGAVHLYA | 406 | 0.768 | 1.212 | 0.09 |
| QAQTAPVQT | | | | |
| PLNGAVHLYA | 407 | 0.740 | 1.206 | 7.9 |
| QAQMAPVQN | | | | |
| PLNGAVHLYA | 408 | 0.499 | 1.202 | 7.06 |
| QAQILGVQN | | | | |
| PLNGAVHLYA | 409 | 0.216 | 1.198 | 0.14 |
| QAQTASVQT | | | | |
| PLNGAVHLYA | 410 | 0.267 | 1.196 | 0.23 |
| QAQTGSLQN | | | | |
| PLNGAVHLYA | 411 | 0.638 | 1.196 | 28.79 |
| QAQTGTVQN | | | | |
| PLNGGVHLYA | 412 | 0.578 | 1.193 | 565.1 |
| QAQTGWVQN | | | | |
| PLNGAVHLYA | 413 | 0.237 | 1.183 | 0.89 |
| QAQTGSVQT | | | | |
| PLNGAVHLYA | 414 | 0.353 | 1.171 | 0.09 |
| QAQTSSVQT | | | | |
| PLNGAVHLYA | 415 | 0.414 | 1.167 | 10.84 |
| QAQTSHVQN | | | | |
| PLNGGVHLYA | 416 | 0.561 | 1.166 | 0.28 |
| QAQTGWDQN | | | | |
| PLNGAVHLYA | 417 | 0.753 | 1.165 | 3.55 |
| QAQRIGVQN | | | | |
| TLNGAVHLYA | 418 | 0.771 | 1.164 | 0.09 |
| QAQTTSVQN | | | | |
| PLNGAVHLYA | 419 | 0.347 | 1.158 | 0.14 |
| QAQTGWGQT | | | | |
| PLNGAVHLYA | 420 | 0.549 | 1.158 | 14.3 |
| QAQSAMVQN | | | | |
| PLNGAVHLYA | 421 | 0.510 | 1.156 | 23.23 |
| QAQTSMVQN | | | | |
| PLNGAVHLYA | 422 | 0.781 | 1.148 | 13.09 |
| QAQSMGVQN | | | | |
| PLNGAVHLYA | 423 | 0.568 | 1.145 | 27.62 |
| QAQSMSVQN | | | | |
| PLNGAVHLYA | 424 | 0.442 | 1.145 | 94.03 |
| QAQTSSVQN | | | | |
| PLNGAVHLYA | 425 | 0.620 | 1.143 | 30.52 |
| QAQTSGVQN | | | | |
| PLNGAVHLYA | 426 | 0.557 | 1.141 | 22.9 |
| QAQTGAVQN | | | | |
| PLNGAVHLYA | 427 | 0.627 | 1.140 | 14.02 |
| QAQVNSVQN | | | | |
| PLNGAVHLYA | 428 | 0.804 | 1.139 | 12.48 |
| QAQVAGVQN | | | | |
| PLNGAVHLYA | 429 | 0.710 | 1.139 | 27.01 |
| QAQIGSVQN | | | | |
| PLDGAVHVYA | 430 | 0.818 | 1.132 | 9.49 |
| QAQTGWVQN | | | | |
| TLNGAVHLYA | 431 | 0.474 | 1.131 | 0.09 |
| QAQTTPVQN | | | | |
| PLNGAVHLYA | 432 | 0.442 | 1.131 | 41.64 |
| QAQLGSVQN | | | | |
| PLNGAVHLYA | 433 | 0.807 | 1.130 | 7.06 |
| QAQVNGVQN | | | | |
| QLNGAVHLYA | 434 | 0.857 | 1.127 | 0.19 |
| QAQLSSVQN | | | | |
| PLNGAVHLYA | 435 | 0.652 | 1.127 | 7.62 |
| QAQLTAVQN | | | | |
| PLNGAVHLYA | 436 | 0.892 | 1.125 | 3.22 |
| QAQVQNVQN | | | | |
| PLNGAVHLYA | 437 | 0.737 | 1.117 | 14.63 |
| QAQTKSVQN | | | | |
| PLNGAVHLYA | 438 | 0.847 | 1.117 | 16.64 |
| QAQSVGVQN | | | | |
| QLNGAVHLYA | 439 | 0.413 | 1.116 | 0.09 |
| QAQLGPVQN | | | | |
| PLNGAVHLYA | 440 | 0.274 | 1.107 | 0.47 |
| QAQTAWVQT | | | | |
| PLNGAVHLYA | 441 | 0.651 | 1.099 | 11.54 |
| QAQNASVQN | | | | |
| PLNGAVHLYA | 442 | 0.661 | 1.098 | 45.57 |
| QAQTSAVQN | | | | |
| ALNGAVHLYA | 443 | 0.530 | 1.097 | 0.09 |
| QAQLAAVQN | | | | |
| PLNGAVHLYD | 444 | 0.721 | 1.097 | 0.09 |
| QAQVSPVQN | | | | |
| PLNGAVHLYA | 445 | 0.442 | 1.095 | 0.09 |
| QAQTMSVKN | | | | |
| PLNGAVHLYA | 446 | 0.650 | 1.092 | 7.7 |
| QAQTANVQN | | | | |
| PLNGAVHLYA | 447 | 0.358 | 1.092 | 5.19 |
| QAQTGWFQN | | | | |
| PLNGNVHLYA | 448 | 0.392 | 1.090 | 74.03 |
| QAQTGWVQN | | | | |
| PLNGSVHLYA | 449 | 0.195 | 1.087 | 0.23 |
| QAQTGWVQS | | | | |
| PLNGAVHLYA | 450 | 0.279 | 1.081 | 0.09 |
| QAQTGGVLN | | | | |
| PLNGAVHLYA | 451 | 0.888 | 1.081 | 16.45 |
| QAQLTSVQN | | | | |
| PLNGAVHLYA | 452 | 0.176 | 1.079 | 19.3 |
| QAQTGWVQT | | | | |
| PLNGAVHLYA | 453 | 0.804 | 1.078 | 10.7 |
| QAQRSQVQN | | | | |
| PLNGAVHIYA | 454 | 0.840 | 1.074 | 0.09 |
| QAQVSPVQN | | | | |
| PLNGSVHLYA | 455 | 0.130 | 1.062 | 0.37 |
| QAQTGWFQN | | | | |
| PLNGAVHLYA | 456 | 0.863 | 1.060 | 3.13 |
| QAQRIPVQN | | | | |
| QLNGAVHLYA | 457 | 0.718 | 1.054 | 0.09 |
| QAQLVPVQN | | | | |
| PLNGAVHLYA | 458 | 0.198 | 1.052 | 2.06 |
| QAQTGWVQD | | | | |
| PLNGAVHLYA | 459 | 0.357 | 1.051 | 0.09 |
| KAQVSSVQN | | | | |
| PLNGAVHLYA | 460 | 0.721 | 1.051 | 11.54 |
| QAQRVEVQN | | | | |
| PLNGAVHLYA | 461 | 0.553 | 1.048 | 56.6 |
| QAQVLPVQN | | | | |
| PLNGSVHLYA | 462 | 0.194 | 1.044 | 0.28 |
| QAQTGWVRN | | | | |
| PLNGTVHLYA | 463 | 0.648 | 1.040 | 0.09 |
| QAQSSPVQN | | | | |
| PLNGAVHLYA | 464 | 0.301 | 1.039 | 0.42 |
| QAQTGGVQK | | | | |
| PLNGAVHLYA | 465 | 0.727 | 1.039 | 14.63 |
| QAQSVAVQN | | | | |
| QLNGAVHLYA | 466 | 0.815 | 1.030 | 0.09 |
| QAQLSGVQN | | | | |
| PLNGAVHLYA | 467 | 0.384 | 1.026 | 11.26 |
| QAQLQQVQN | | | | |
| PLNGAVHLYA | 468 | 0.547 | 1.025 | 32.06 |
| QAQLSTVQN | | | | |
| PLNGAVHLYA | 469 | 0.813 | 1.025 | 18.09 |
| QAQSAAVQN | | | | |
| SLNGAVHLYA | 470 | 0.774 | 1.014 | 0.14 |
| QAQVSAVQN | | | | |
| PLNGAVHLYA | 471 | 0.432 | 1.013 | 20.47 |
| QAQRTSVQN | | | | |
| PLNGAVHLYA | 472 | 0.877 | 1.012 | 5.47 |
| QAQTQNVQN | | | | |
| PLNGAVHLYA | 473 | 0.877 | 1.007 | 4.81 |
| QAQVNAVQN | | | | |
| PLNGAVHLYA | 474 | 0.585 | 1.006 | 0.09 |
| QAQTVAVQT | | | | |
| PLNGAVHLYA | 475 | 0.487 | 1.005 | 20.75 |
| QAQLMSVQN | | | | |
| PLNGAVHLYA | 476 | 0.179 | 1.000 | 15655 |
| QAQTGWVQN | | | | |
| PLNGAVHLYA | 477 | 0.354 | 0.999 | 0.09 |
| HAQTGWVQH | | | | |
| PLNGAVHLYA | 478 | 0.283 | 0.997 | 7.48 |
| QAQTGWVQH | | | | |
| PLNGAVHHYA | 479 | 0.408 | 0.993 | 0.09 |
| QAQTMSVQN | | | | |
| PLNGAVHLYA | 480 | 0.228 | 0.978 | 3.08 |
| QAQTGWVQS | | | | |
| PLNGAVHLYD | 481 | 0.409 | 0.976 | 0.09 |
| QAQTTPVQN | | | | |
| PLNGAVHLYA | 482 | 0.837 | 0.973 | 3.69 |
| QAQTMMVQN | | | | |
| PLNGAVHLYA | 483 | 0.293 | 0.971 | 0.14 |
| QAQTGGVQS | | | | |
| PLNGAVNLYA | 484 | 0.499 | 0.969 | 0.14 |
| QAQTSPVQN | | | | |
| ALNGAVHLYA | 485 | 0.322 | 0.968 | 47.95 |
| QAQTGLVQN | | | | |
| ALNGAVHLYA | 486 | 0.247 | 0.964 | 0.09 |
| QAQVATVQN | | | | |
| PLNGAVHLYA | 487 | 0.258 | 0.959 | 0.23 |
| QAQTGSFQN | | | | |
| PLNGAVHLYA | 488 | 0.422 | 0.958 | 27.01 |
| QAQRSVVQN | | | | |
| PLNGAVHLYA | 489 | 0.224 | 0.957 | 0.65 |
| QAQTGSVQK | | | | |
| PLNGAVHLYA | 490 | 0.503 | 0.954 | 16.31 |
| QAQTQLVQN | | | | |
| PLNGAVHLYA | 491 | 0.252 | 0.954 | 0.19 |
| QAQTGGVHN | | | | |
| PLNGAVHLYA | 492 | 0.328 | 0.953 | 11.92 |
| QAQRTPVQN | | | | |
| PLNGAVHLYA | 493 | 0.852 | 0.952 | 5.33 |
| QAQRQQVQN | | | | |
| PLNGAVHLYA | 494 | 0.321 | 0.952 | 0.19 |
| QAQTGSVRN | | | | |
| PLNGAVHLYA | 495 | 0.758 | 0.946 | 11.87 |
| QAQVQGVQN | | | | |
| TLNGAVHLYA | 496 | 0.854 | 0.942 | 0.14 |
| QAQVSPVQN | | | | |
| PLNGAVHLYA | 497 | 0.205 | 0.942 | 0.23 |
| QAQPGWVQT | | | | |
| PLNGAVHLYA | 498 | 0.407 | 0.941 | 17.25 |
| QAQSTQVQN | | | | |
| PLNGSVHLYA | 499 | 0.247 | 0.939 | 0.09 |
| QAQTGWVEN | | | | |
| PLNGAVHLYA | 500 | 0.267 | 0.934 | 22.29 |
| QAQQSPVQN | | | | |
| PLNGAVHLYA | 501 | 0.517 | 0.934 | 8.23 |
| QAQRYSVQN | | | | |
| PLNGAVHLYA | 502 | 0.267 | 0.930 | 9.77 |
| QAQTGWDQN | | | | |
| PLNGAVHLYA | 503 | 0.774 | 0.930 | 4.67 |
| QAQTQRVQN | | | | |
| ALNGAVHLYA | 504 | 0.330 | 0.926 | 0.23 |
| QAQTGWVRN | | | | |
| PLNGAVHLYA | 505 | 0.370 | 0.923 | 171 |
| QPQTGWVQN | | | | |
| ALNGAVHLYA | 506 | 0.516 | 0.920 | 0.09 |
| QAQVSGVQN | | | | |
| PLNGAVHLYA | 507 | 0.813 | 0.920 | 17.01 |
| QAQSAGVQN | | | | |
| PLNGAVHLYA | 508 | 0.734 | 0.918 | 0.09 |
| QAQSAPVQT | | | | |
| PLNGAVHLYA | 509 | 0.499 | 0.914 | 13.97 |
| QAQRQSVQN | | | | |
| PLNGAVHLYA | 510 | 0.591 | 0.906 | 20.42 |
| QAQSQPVQN | | | | |
| PLNGAVHLYA | 511 | 0.570 | 0.906 | 0.09 |
| QAQVASVKN | | | | |
| PLNGAVHLYA | 512 | 0.403 | 0.904 | 0.09 |
| QAQTAWVRN | | | | |
| SLNGAVHLYA | 513 | 0.491 | 0.904 | 0.09 |
| QAQTMQVQN | | | | |
| PLNGAVNLYA | 514 | 0.664 | 0.902 | 0.09 |
| QAQSSPVQN | | | | |
| PLNGAVHLYA | 515 | 0.318 | 0.901 | 0.23 |
| QAQTGGGQN | | | | |
| PLNGAVHLYD | 516 | 0.515 | 0.898 | 0.09 |
| QAQVASVQN | | | | |
| PLNGAVHLYA | 517 | 0.709 | 0.897 | 45.94 |
| QAQSTSVQN | | | | |
| PLNGTVHLYA | 518 | 0.403 | 0.894 | 0.23 |
| QAQTGWVQH | | | | |
| QLNGAVHLYA | 519 | 0.718 | 0.892 | 0.42 |
| QAQTSSVQN | | | | |
| PLDGAVHLYA | 520 | 0.613 | 0.892 | 0.09 |
| QAQTGWVHN | | | | |
| PLNGTVHLYA | 521 | 0.397 | 0.888 | 0.33 |
| QAQTGWVQK | | | | |
| PLNGAVHLYA | 522 | 0.369 | 0.888 | 20.28 |
| QAQVLGVQN | | | | |
| PLNGAVHLYA | 523 | 0.640 | 0.887 | 5.7 |
| QAQNQPVQN | | | | |
| PLNGAVHLYA | 524 | 0.605 | 0.885 | 21.08 |
| QAQLGTVQN | | | | |
| PLNGAVHLYA | 525 | 0.637 | 0.882 | 7.57 |
| QAQRAGVQN | | | | |
| PVNGAVHLYA | 526 | 0.837 | 0.882 | 42.25 |
| QAQTGLVQN | | | | |
| PLNGGVHLYA | 527 | 0.529 | 0.881 | 0.33 |
| QAQTGWVQH | | | | |
| PLNGAVHLYA | 528 | 0.677 | 0.879 | 116.4 |
| QAQTGRVQN | | | | |
| PLNGAVHLYA | 529 | 0.266 | 0.876 | 0.56 |
| QAQTGGVKN | | | | |
| PLNGAVHLYA | 530 | 0.251 | 0.874 | 0.14 |
| QAQTAWLQN | | | | |
| PLNGAVHLYA | 531 | 0.269 | 0.870 | 0.14 |
| QAQTRWVQK | | | | |
| PLNGAVHLYA | 532 | 0.699 | 0.858 | 0.09 |
| QAQLAPVKN | | | | |
| PLNGAVHHYA | 533 | 0.390 | 0.855 | 0.09 |
| QAQTSSVQN | | | | |
| PLNGAVHLYA | 534 | 0.402 | 0.851 | 0.14 |
| QAQTGSVQD | | | | |
| PLNGAVHLYA | 535 | 0.557 | 0.849 | 27.67 |
| QAQTGMVQN | | | | |
| PLNGAVHLYA | 536 | 0.647 | 0.848 | 15.14 |
| QAQLSNVQN | | | | |
| PLNGAVHLYA | 537 | 0.502 | 0.844 | 14.39 |
| QAQRLPVQN | | | | |
| PLNGAVHLYA | 538 | 0.782 | 0.842 | 7.52 |
| QAQRQGVQN | | | | |
| PLNGAVHLYA | 539 | 0.563 | 0.839 | 0.09 |
| QAQSTPVQT | | | | |
| QLNGNVHLYA | 540 | 0.844 | 0.838 | 5.56 |
| QAQTGWVQN | | | | |
| PLNGGVHLYA | 541 | 0.559 | 0.835 | 0.84 |
| QAQTGWVQT | | | | |
| PLNGAVHLYA | 542 | 0.470 | 0.833 | 0.09 |
| QAQTTPVKN | | | | |
| PLNGAVHLYA | 543 | 0.700 | 0.832 | 18.65 |
| QAQRLTVQN | | | | |
| PLNGAVHLYA | 544 | 0.373 | 0.830 | 7.52 |
| QAQTLRVQN | | | | |
| PLNGAVHLYA | 545 | 0.742 | 0.830 | 7.34 |
| QAQSAFVQN | | | | |
| SLNGAVHLYA | 546 | 0.586 | 0.824 | 0.09 |
| QAQTNSVQN | | | | |
| PLNGAVHLYA | 547 | 0.658 | 0.824 | 6.92 |
| QAQSVQVQN | | | | |
| PLDGAVHLYA | 548 | 0.568 | 0.823 | 0.09 |
| QAQTAAVQN | | | | |
| SLNGAVHLYA | 549 | 0.532 | 0.821 | 0.09 |
| QAQLMPVQN | | | | |
| PLDGAVHLYA | 550 | 0.621 | 0.820 | 0.47 |
| QAQTGWVQT | | | | |
| PLNGAVHLYA | 551 | 0.672 | 0.819 | 49.77 |
| QAQLLPVQN | | | | |
| PLNGAVHLYA | 552 | 0.738 | 0.815 | 6.64 |
| QAQRTQVQN | | | | |
| PLNGAVHLYA | 553 | 0.254 | 0.813 | 0.79 |
| QAQTGSDQN | | | | |
| PLNGAVHLYA | 554 | 0.430 | 0.808 | 0.09 |
| QAQVASDQN | | | | |
| PLNGAVNLYA | 555 | 0.744 | 0.805 | 0.09 |
| QAQLAPVQN | | | | |
| PLNGSVHLYA | 556 | 0.160 | 0.804 | 0.37 |
| QAQTGWLQN | | | | |
| PLNGTVHLYA | 557 | 0.253 | 0.801 | 500.6 |
| QAQTGWVQN | | | | |
| PLNGAVHLYA | 558 | 0.857 | 0.801 | 0.14 |
| QAQVSPVKN | | | | |
| PLNGAVHLYA | 559 | 0.480 | 0.801 | 1.22 |
| QAQLPPVQN | | | | |
| PLNGAVHLYA | 560 | 0.521 | 0.794 | 0.14 |
| QAQSSPVQT | | | | |
| PLNGAVHLYA | 561 | 0.585 | 0.790 | 8.88 |
| QAQAQPVQN | | | | |
| PLNGDVHLYA | 562 | 0.434 | 0.789 | 0.09 |
| QAQTTSVQN | | | | |
| PLNGAVHLYA | 563 | 0.392 | 0.788 | 0.75 |
| QAQTPQVQN | | | | |
| PLNGAVHLYA | 564 | 0.571 | 0.787 | 29.35 |
| QAQTSWVQN | | | | |
| PLNGAVHLYA | 565 | 0.254 | 0.784 | 0.51 |
| QAQTGGDQN | | | | |
| PLNGAVHLYA | 566 | 0.547 | 0.782 | 0.14 |
| QAQLSPVKN | | | | |
| PLNGAVHLYA | 567 | 0.603 | 0.782 | 0.09 |
| QAQSSPVKN | | | | |
| PLNGAVHLYA | 568 | 0.727 | 0.781 | 8.41 |
| QAQNTTVQN | | | | |
| PLDGAVHLYA | 569 | 0.716 | 0.780 | 0.19 |
| QAQTGWVQH | | | | |
| PLNGAVHLYA | 570 | 0.823 | 0.773 | 6.31 |
| QAQTTRVQN | | | | |
| PLDGAVHLYA | 571 | 0.722 | 0.767 | 0.09 |
| QAQTGWLQN | | | | |
| PLNGSVHLYA | 572 | 0.681 | 0.757 | 8.32 |
| QPQTGWVQN | | | | |
| PLNGAVHLYS | 573 | 0.794 | 0.755 | 25.1 |
| QAQTGLVQN | | | | |
| PLNGAVHLYA | 574 | 0.654 | 0.755 | 14.16 |
| QAQTQWVQN | | | | |
| PLNGSVHLYA | 575 | 0.187 | 0.754 | 0.33 |
| QAQTGWVQI | | | | |
| PLNGAVHLYA | 576 | 0.259 | 0.753 | 0.28 |
| QAQTGSVHN | | | | |
| ALNGAVHLYA | 577 | 0.367 | 0.751 | 1129 |
| QAQTGWVQN | | | | |
| PLNGTVHLYA | 578 | 0.224 | 0.749 | 0.14 |
| QAQTGWVRN | | | | |
| PLNGAVHLYA | 579 | 0.240 | 0.749 | 0.23 |
| QAQTGGLQN | | | | |
| SLNGAVHLYA | 580 | 0.712 | 0.748 | 0.09 |
| QAQVSGVQN | | | | |
| PLNGAVHLYA | 581 | 0.404 | 0.747 | 0.37 |
| QAQTAWVQK | | | | |
| PLNGAVHLYA | 582 | 0.660 | 0.747 | 0.19 |
| QAQTGRVQT | | | | |
| TLNGAVHLYA | 583 | 0.754 | 0.739 | 0.09 |
| QAQSAPVQN | | | | |
| PLNGAVHLYD | 584 | 0.449 | 0.738 | 0.09 |
| QAQTVAVQN | | | | |
| QLNGAVHLYA | 585 | 0.892 | 0.735 | 48.7 |
| QAQTGSVQN | | | | |
| PLNGAVHLYA | 586 | 0.858 | 0.733 | 5.37 |
| QAQNTQVQN | | | | |
| PLNGAVHLYA | 587 | 0.512 | 0.729 | 5.05 |
| QAQTGWLQN | | | | |
| PINGAVHLYA | 588 | 0.684 | 0.721 | 45.57 |
| QAQTGLVQN | | | | |
| PLDGAVHLYA | 589 | 0.600 | 0.719 | 0.09 |
| QAQTGWVQI | | | | |
| PLNGSVHLYA | 590 | 0.369 | 0.713 | 0.09 |
| QAQTGSVQT | | | | |
| PLNGAVQLYA | 591 | 0.460 | 0.711 | 0.09 |
| QAQTSSVQN | | | | |
| PLNGAVHLYA | 592 | 0.658 | 0.707 | 5.61 |
| QAQTYAVQN | | | | |
| PLNGAVHLYA | 593 | 0.575 | 0.703 | 81.37 |
| QAQTLSVQN | | | | |
| PLNGAVHLYA | 594 | 0.874 | 0.703 | 12.15 |
| QAQNLPVQN | | | | |
| PLNGAVHLYA | 595 | 0.328 | 0.702 | 0.09 |
| QAQTSSDQN | | | | |
| PLNGAVHLYA | 596 | 0.736 | 0.702 | 23.93 |
| QAQATSVQN | | | | |
| TLNGAVHLYA | 597 | 0.353 | 0.701 | 0.23 |
| QAQLSPVQN | | | | |
| PLNGAVHLYA | 598 | 0.271 | 0.700 | 0.14 |
| QAQTAWFQN | | | | |
| TLNGAVHLYA | 599 | 0.801 | 0.700 | 0.09 |
| QAQVTPVQN | | | | |
| TLNGAVHLYA | 600 | 0.202 | 0.696 | 0.09 |
| QAQTAAVQN | | | | |
| PLNGAVHLYA | 601 | 0.801 | 0.693 | 7.76 |
| QAQATQVQN | | | | |
| RLDGAVHLYA | 602 | 0.861 | 0.693 | 8.37 |
| QAQTGWVQN | | | | |
| QLNGAVHLYA | 603 | 0.840 | 0.691 | 0.09 |
| QAQTGWVEN | | | | |
| PLNGAVHLYA | 604 | 0.278 | 0.691 | 0.19 |
| QAQVSSVKN | | | | |
| PLNGAVHLYA | 605 | 0.582 | 0.690 | 94.87 |
| QAQLSSVQN | | | | |
| PLNGAVHLYA | 606 | 0.209 | 0.689 | 0.33 |
| QAQTGSGQN | | | | |
| PLNGAVHLYA | 607 | 0.441 | 0.686 | 0.09 |
| QAQLQPVQT | | | | |
| PINGAVHLYA | 608 | 0.805 | 0.686 | 0.09 |
| QAQLSSVQN | | | | |
| PINGAVHLYA | 609 | 0.313 | 0.684 | 0.09 |
| QAQTVAVQN | | | | |
| PLNGAVHLYA | 610 | 0.546 | 0.680 | 0.09 |
| QAQVSAVKN | | | | |
| SLNGAVHLYA | 611 | 0.461 | 0.680 | 0.23 |
| QAQTVAVQN | | | | |
| PLNGAVHLYA | 612 | 0.882 | 0.680 | 22.29 |
| QAQTQAVQN | | | | |
| QLNGAVHLYA | 613 | 0.398 | 0.679 | 0.28 |
| QAQTAQVQN | | | | |
| PLNGAVHLYD | 614 | 0.309 | 0.676 | 0.14 |
| QAQTMSVQN | | | | |
| PLNGAVHLYA | 615 | 0.341 | 0.675 | 0.19 |
| QPQTGWVQK | | | | |
| PLNGAVHLYA | 616 | 0.855 | 0.672 | 30.47 |
| QAQVMPVQN | | | | |
| PLNGAVHLYA | 617 | 0.208 | 0.669 | 0.19 |
| QAQTVSVQK | | | | |
| PLNGAVHLYA | 618 | 0.410 | 0.664 | 0.09 |
| QAQTAWAQN | | | | |
| PLNGAVHLYA | 619 | 0.764 | 0.664 | 12.06 |
| QAQNALVQN | | | | |
| TLNGAVHLYA | 620 | 0.217 | 0.663 | 0.09 |
| QAQVSAVQN | | | | |
| PLKGAVHLYA | 621 | 0.611 | 0.663 | 0.09 |
| QAQTTPVQN | | | | |
| QLNGSVHLYA | 622 | 0.776 | 0.657 | 96.83 |
| QAQTGWVQN | | | | |
| SLNGAVHLYA | 623 | 0.307 | 0.650 | 0.09 |
| QAQVATVQN | | | | |
| PLNGAVHLYA | 624 | 0.876 | 0.650 | 9.39 |
| QAQRMLVQN | | | | |
| PLNGAVHLYA | 625 | 0.589 | 0.649 | 0.09 |
| QAQTVAVKN | | | | |
| PLNGAVHLYD | 626 | 0.523 | 0.649 | 0.14 |
| QAQTTSVQN | | | | |
| PLNGAVHLYA | 627 | 0.725 | 0.647 | 11.92 |
| QAQRQLVQN | | | | |
| PLNGAVHLYA | 628 | 0.754 | 0.645 | 28.79 |
| QAQTLGVQN | | | | |
| PLNGAVHLYA | 629 | 0.889 | 0.645 | 0.09 |
| QAQLGPVQT | | | | |
| PLNGGVHLYA | 630 | 0.554 | 0.644 | 0.14 |
| QAQTGWVQD | | | | |
| PLNGAVHLYA | 631 | 0.205 | 0.640 | 0.98 |
| QAQTGSVKN | | | | |
| PLNGAVNLYA | 632 | 0.332 | 0.635 | 0.09 |
| QAQLASVQN | | | | |
| PLNGAVHLYD | 633 | 0.307 | 0.634 | 0.14 |
| QAQVSSVQN | | | | |
| PLNGAVHLYA | 634 | 0.355 | 0.632 | 0.14 |
| QAQTMSDQN | | | | |
| PLNGAVHLYA | 635 | 0.337 | 0.630 | 17.48 |
| QAQTGWVQK | | | | |
| PLNGAVHLYA | 636 | 0.203 | 0.630 | 0.09 |
| QAQLASVKN | | | | |
| PLNGGVHLYA | 637 | 0.490 | 0.628 | 0.23 |
| QAQTGWGQN | | | | |
| QLNGSVHLYA | 638 | 0.844 | 0.627 | 0.09 |
| QAQTGWVQT | | | | |
| QLNGAVHLYA | 639 | 0.845 | 0.626 | 0.09 |
| QAQTTGVQN | | | | |
| PLNGAVHLYA | 640 | 0.602 | 0.625 | 39.96 |
| QAQTGFVQN | | | | |
| PLNGAVHLYA | 641 | 0.536 | 0.625 | 42.62 |
| QAQSALVQN | | | | |
| ALNGAVHLYA | 642 | 0.278 | 0.624 | 0.75 |
| QAQTGWDQN | | | | |
| QLNGAVHLYA | 643 | 0.636 | 0.623 | 0.09 |
| QAQISGVQN | | | | |
| QLNGAVHLYA | 644 | 0.520 | 0.623 | 0.14 |
| QAQRGSVQN | | | | |
| PLNGAVHLYA | 645 | 0.270 | 0.622 | 0.19 |
| QAQTGWVKT | | | | |
| PLNGAVHLYA | 646 | 0.554 | 0.619 | 27.62 |
| QAQSQLVQN | | | | |
| PLNGSVHLYA | 647 | 0.202 | 0.617 | 0.33 |
| QAQTGWGQN | | | | |
| QLNGAVHLYA | 648 | 0.632 | 0.616 | 0.14 |
| QAQTGWVPN | | | | |
| PLNGAVHLYA | 649 | 0.305 | 0.615 | 0.33 |
| KAQTGWVKN | | | | |
| PLNGAVHLYA | 650 | 0.164 | 0.615 | 0.09 |
| HAQTGWGQN | | | | |
| ALNGAVHLYA | 651 | 0.330 | 0.613 | 0.61 |
| QAQTGWVQH | | | | |
| PLNGSVHLYA | 652 | 0.585 | 0.612 | 0.14 |
| QAQIASVQN | | | | |
| ALNGAVHLYA | 653 | 0.416 | 0.611 | 0.14 |
| QAQTAMVQN | | | | |
| TLNGAVHLYA | 654 | 0.316 | 0.610 | 0.09 |
| QAQTMAVQN | | | | |
| PHNGAVHLYA | 655 | 0.476 | 0.609 | 0.09 |
| QAQVSSVQN | | | | |
| PLNGAVQLYA | 656 | 0.693 | 0.606 | 0.09 |
| QAQTAPVQN | | | | |
| PLNGSVHLYA | 657 | 0.282 | 0.605 | 0.09 |
| QAQKGSVQN | | | | |
| PLNGAVHLYA | 658 | 0.574 | 0.605 | 39.3 |
| QAQLSAVQN | | | | |
| PINGAVHLYA | 659 | 0.489 | 0.605 | 0.09 |
| QAQVSAVQN | | | | |
| PLNGSVHLYA | 660 | 0.514 | 0.604 | 43.37 |
| QAQTGLVQN | | | | |
| PLNGAVHIYA | 661 | 0.223 | 0.604 | 0.14 |
| QAQTGWFQN | | | | |
| SLNGAVHLYA | 662 | 0.293 | 0.603 | 0.09 |
| QAQIASVQN | | | | |
| PLNGAVHLYA | 663 | 0.220 | 0.601 | 0.14 |
| QEQTVSVQN | | | | |
| PLNGAVHLYA | 664 | 0.737 | 0.601 | 0.09 |
| QAQTMQVQT | | | | |
| HLNGAVHLYA | 665 | 0.338 | 0.600 | 56.78 |
| QAQTGWVQN | | | | |
| PLNGAVHLYA | 666 | 0.400 | 0.600 | 24.02 |
| QAQTGQVQN | | | | |
| QLNGAVHLYA | 667 | 0.596 | 0.599 | 0.19 |
| QAQTGWVQD | | | | |
| PLNGAVHLYD | 668 | 0.526 | 0.599 | 0.09 |
| QAQVAQVQN | | | | |
| PLNGAVHLYA | 669 | 0.515 | 0.598 | 0.19 |
| QAQTSPVKN | | | | |
| PLNGAIHLYA | 670 | 0.383 | 0.595 | 0.09 |
| QAQTSPVQN | | | | |
| PLNGSVHLYA | 671 | 0.578 | 0.595 | 0.19 |
| QAQTAMVQN | | | | |
| PLNGAVHLYA | 672 | 0.370 | 0.594 | 0.09 |
| QAQLSQVQT | | | | |
| PLNGAVHLYA | 673 | 0.310 | 0.594 | 0.19 |
| QAQTGSVLN | | | | |
| PLNGAIHLYA | 674 | 0.428 | 0.593 | 0.09 |
| QAQTASVQN | | | | |
| PLNGAVHLYA | 675 | 0.785 | 0.592 | 6.68 |
| QAQTNGVQN | | | | |
| PLNGSVHLYA | 676 | 0.334 | 0.591 | 0.14 |
| QAQTGWVQY | | | | |
| PLNGALHLYA | 677 | 0.463 | 0.590 | 0.09 |
| QAQTSPVQN | | | | |
| ALNGAVHLYA | 678 | 0.399 | 0.589 | 1.59 |
| QAQTGWVQT | | | | |
| PLNGAVHLYA | 679 | 0.193 | 0.586 | 0.47 |
| QAQTGWVKK | | | | |
| QLNGAVHLYA | 680 | 0.630 | 0.584 | 1450 |
| QAQTGWVQN | | | | |
| PLNGDVHLYA | 681 | 0.630 | 0.580 | 0.09 |
| QAQTVAVQN | | | | |
| PLNGGVHLYA | 682 | 0.501 | 0.580 | 0.47 |
| QAQPGWVQN | | | | |
| PLNGAVHLYA | 683 | 0.897 | 0.579 | 7.66 |
| QAQAAAVQN | | | | |
| PLNGAVHLYA | 684 | 0.618 | 0.579 | 19.72 |
| QAQSAVVQN | | | | |
| TLNGAVHLYA | 685 | 0.646 | 0.578 | 0.09 |
| QAQRSSVQN | | | | |
| PLNGAVHLYA | 686 | 0.214 | 0.578 | 0.14 |
| QAQPGWVQH | | | | |
| PLDGAVHLYA | 687 | 0.214 | 0.577 | 0.09 |
| QAQTLSVQN | | | | |
| PLNGAVHLYA | 688 | 0.310 | 0.575 | 0.19 |
| QAQTASDQN | | | | |
| PLNGAVHLYA | 689 | 0.633 | 0.575 | 53.51 |
| QAQLSGVQN | | | | |
| TLNGAVHLYA | 690 | 0.463 | 0.572 | 0.14 |
| QAQTVAVQN | | | | |
| PLNGAVHLYA | 691 | 0.293 | 0.572 | 0.09 |
| QEQVASVQN | | | | |
| PVNGAVHLYA | 692 | 0.873 | 0.572 | 0.09 |
| QAQTGWAQN | | | | |
| PLNGAVHLYA | 693 | 0.567 | 0.570 | 14.07 |
| QAQSSRVQN | | | | |
| PLNGSVHLYA | 694 | 0.551 | 0.569 | 0.19 |
| QAQLGSVQN | | | | |
| PLNGAVHLYA | 695 | 0.565 | 0.568 | 1.22 |
| QAQSPPVQN | | | | |
| PLNGAVHHYA | 696 | 0.605 | 0.567 | 0.14 |
| QAQTTSVQN | | | | |
| PLNGAVHLYD | 697 | 0.832 | 0.567 | 0.09 |
| QAQSTPVQN | | | | |
| QLNGAVHLYA | 698 | 0.529 | 0.567 | 0.37 |
| QAQTGWVRN | | | | |
| PLNGAVHLYA | 699 | 0.597 | 0.563 | 0.09 |
| QAQVQPVQT | | | | |
| PLNGAVHLYA | 700 | 0.416 | 0.562 | 0.14 |
| QAQTSSVKN | | | | |
| PLNGALHLYA | 701 | 0.301 | 0.560 | 0.09 |
| QAQTASVQN | | | | |
| PLNGAVHHYA | 702 | 0.731 | 0.558 | 0.09 |
| QAQSAPVQN | | | | |
| PLNGSVHLYA | 703 | 0.169 | 0.556 | 0.47 |
| QAQTGWVHN | | | | |
| PLNGAVHLYA | 704 | 0.519 | 0.556 | 0.09 |
| QAQLSPLQN | | | | |
| PLNGAVHLYA | 705 | 0.841 | 0.554 | 0.09 |
| KAQVTPVQN | | | | |
| PLNGAVNLYA | 706 | 0.278 | 0.554 | 0.09 |
| QAQTQPVQN | | | | |
| SLNGAVHLYA | 707 | 0.518 | 0.553 | 92.07 |
| QAQTGLVQN | | | | |
| PLDGSVHLYA | 708 | 0.366 | 0.552 | 0.09 |
| QAQTGGVQN | | | | |
| PLNGAVHLYD | 709 | 0.528 | 0.552 | 0.09 |
| QAQTLPVQN | | | | |
| PLNGAVHLYA | 710 | 0.441 | 0.551 | 0.09 |
| QAQVSQVQK | | | | |
| PLNGAVHLYA | 711 | 0.425 | 0.551 | 13.97 |
| QAQLVGVQN | | | | |
| PLNGAVHLYA | 712 | 0.390 | 0.549 | 0.09 |
| QEQTSSVQN | | | | |
| PLNGAVHIYA | 713 | 0.435 | 0.549 | 0.09 |
| QAQTVAVQN | | | | |
| PLNGAVNLYA | 714 | 0.831 | 0.548 | 0.14 |
| QAQVTPVQN | | | | |
| TLNGAVHLYA | 715 | 0.252 | 0.547 | 0.19 |
| QAQTMSVQN | | | | |
| TLNGAVHLYA | 716 | 0.341 | 0.541 | 0.09 |
| QAQTLAVQN | | | | |
| PINGAVHLYA | 717 | 0.341 | 0.540 | 0.09 |
| QAQVAQVQN | | | | |
| PLNGQVHLYA | 718 | 0.885 | 0.540 | 12.57 |
| QAQTGWVQN | | | | |
| QLNGAVHLYA | 719 | 0.892 | 0.540 | 39.63 |
| QAQTGGVQN | | | | |
| PLNGAVHLYA | 720 | 0.542 | 0.539 | 0.09 |
| QAQTLSVKN | | | | |
| PLNGTVHLYA | 721 | 0.331 | 0.538 | 0.37 |
| QAQTGWDQN | | | | |
| PLNGAVHLYA | 722 | 0.476 | 0.537 | 0.14 |
| QAQTGWFQK | | | | |
| ALNGAVHLYA | 723 | 0.447 | 0.536 | 0.09 |
| QAQTSKVQN | | | | |
| ALNGTVHLYA | 724 | 0.712 | 0.535 | 49.16 |
| QAQTGWVQN | | | | |
| PLNGAVPLYA | 725 | 0.256 | 0.534 | 0.14 |
| QAQTGWVQH | | | | |
| PLNGAVHLYA | 726 | 0.355 | 0.533 | 0.23 |
| QAKTGWVQK | | | | |
| PLNGAVHLYA | 727 | 0.492 | 0.531 | 1.12 |
| QAQTPAVQN | | | | |
| PLNGAVHLYA | 728 | 0.538 | 0.531 | 0.14 |
| QAQTVGVKN | | | | |
| PLNGAVHLYA | 729 | 0.307 | 0.530 | 0.14 |
| QEQTGWFQN | | | | |
| PLNGAVHLYA | 730 | 0.569 | 0.530 | 0.09 |
| QAQKGWDQN | | | | |
| PLNGAVHLYD | 731 | 0.605 | 0.529 | 0.09 |
| QAQTSAVQN | | | | |
| PLDGAVHLYA | 732 | 0.657 | 0.527 | 0.37 |
| QAQTGWVQK | | | | |
| PLNGAVHLYA | 733 | 0.290 | 0.524 | 0.09 |
| QAQTAWVLN | | | | |
| PLNGSVHLYA | 734 | 0.172 | 0.524 | 0.28 |
| QAQTGWVLN | | | | |
| QLNGAVHLYA | 735 | 0.605 | 0.524 | 1.68 |
| QAQTGWVQT | | | | |
| PLNGAVHLYA | 736 | 0.699 | 0.524 | 0.98 |
| QAQVPPVQN | | | | |
| PVNGAVHLYA | 737 | 0.734 | 0.522 | 0.09 |
| QAQVQSVQN | | | | |
| QLNGAVHLYA | 738 | 0.473 | 0.522 | 0.09 |
| QAQTKSVQN | | | | |
| PVNGAVHLYA | 739 | 0.825 | 0.522 | 0.23 |
| QAQTSSVQN | | | | |
| PLNGAVHLYA | 740 | 0.797 | 0.520 | 11.45 |
| QAQATGVQN | | | | |
| TLNGAVHLYA | 741 | 0.553 | 0.518 | 0.09 |
| QAQLSQVQN | | | | |
| PLDGAVHLYA | 742 | 0.486 | 0.517 | 0.37 |
| QAQTVSVQN | | | | |
| SLNGAVHLYA | 743 | 0.421 | 0.514 | 0.09 |
| QAQTKPVQN | | | | |
| PLKGAVHLYA | 744 | 0.705 | 0.514 | 0.09 |
| QAQSAPVQN | | | | |
| PLNGAVHLYA | 745 | 0.275 | 0.514 | 0.19 |
| QAKTGWVKN | | | | |
| PLNGSVHLYA | 746 | 0.680 | 0.513 | 0.09 |
| QAQTLGVQN | | | | |
| PLKGAVHLYA | 747 | 0.626 | 0.512 | 0.09 |
| QAQLAPVQN | | | | |
| PLNGSVHLYA | 748 | 0.207 | 0.512 | 1.17 |
| QAQTGWVQK | | | | |
| PLNGAVHLYA | 749 | 0.295 | 0.511 | 0.28 |
| QAQTGGVQI | | | | |
| PLNGAVHLYA | 750 | 0.440 | 0.510 | 0.37 |
| QPQTGWVQT | | | | |
| PLNGAVHLYA | 751 | 0.196 | 0.508 | 4.02 |
| QAQTGWVLN | | | | |
| PLNGAVHLYD | 752 | 0.611 | 0.507 | 0.14 |
| QAQLTPVQN | | | | |
| QLNGAVHLYA | 753 | 0.558 | 0.507 | 0.28 |
| QAQTATVQN | | | | |
| PLNGAVHLYA | 754 | 0.359 | 0.505 | 0.19 |
| QAQTAWGQN | | | | |
| PLNGAVHLYA | 755 | 0.273 | 0.505 | 6.78 |
| QAQTGWVHN | | | | |
| PLKGAVHLYA | 756 | 0.556 | 0.505 | 0.09 |
| QAQSSPVQN | | | | |
| PLNGAVHLYA | 757 | 0.374 | 0.505 | 0.09 |
| QAQLGSVQT | | | | |
| PLNGAVHLYA | 758 | 0.656 | 0.505 | 7.62 |
| QAQSANVQN | | | | |
| PLNGGVHLYA | 759 | 0.866 | 0.503 | 23.27 |
| QAQTGSVQN | | | | |
| PLNGAVHLYD | 760 | 0.433 | 0.503 | 0.09 |
| QAQRSSVQN | | | | |
| PLNGAVHLYA | 761 | 0.164 | 0.502 | 0.19 |
| QAQTGGVQD | | | | |
| PLNGAVHLYA | 762 | 0.271 | 0.502 | 0.09 |
| QAQTMAVKN | | | | |
| PLNGAVHLYA | 763 | 0.325 | 0.501 | 0.23 |
| QAQTASVKN | | | | |
| PLNGAVHLYA | 764 | 0.531 | 0.501 | 0.14 |
| QAQSSPVQK | | | | |
| PLNGAVHLYA | 765 | 0.275 | 0.499 | 21.68 |
| QAQTGWVKN | | | | |
| ALNGAVHLYA | 766 | 0.735 | 0.499 | 25.05 |
| QAQTGGVQN | | | | |
| PLNGAVHLYA | 767 | 0.676 | 0.498 | 0.09 |
| QAQTGTVQT | | | | |
| PLNGAVHLYA | 768 | 0.476 | 0.495 | 7.43 |
| QAQTGWVQI | | | | |
| PINGAVHLYA | 769 | 0.382 | 0.494 | 0.09 |
| QAQTSLVQN | | | | |
| PLNGAVHIYA | 770 | 0.502 | 0.492 | 0.09 |
| QAQVAQVQN | | | | |
| PLNGAVHLYA | 771 | 0.279 | 0.491 | 0.19 |
| QAQTVWVKN | | | | |
| TLNGAVHLYA | 772 | 0.827 | 0.491 | 0.09 |
| QAQLMPVQN | | | | |
| PLNGAVHIYA | 773 | 0.672 | 0.490 | 0.09 |
| QAQTAPVQN | | | | |
| PLYGAVHLYA | 774 | 0.240 | 0.488 | 0.09 |
| QAQTASVQN | | | | |
| TLNGAVHLYA | 775 | 0.643 | 0.488 | 0.09 |
| QAQLGPVQN | | | | |
| PLDGAVHLYA | 776 | 0.637 | 0.487 | 0.42 |
| QAQTGWVKN | | | | |
| PLKGAVHLYA | 777 | 0.669 | 0.487 | 0.14 |
| QAQVSPVQN | | | | |
| PLNGAVHLYA | 778 | 0.563 | 0.486 | 9.95 |
| QAQSQQVQN | | | | |
| PLNGAVDLYA | 779 | 0.497 | 0.484 | 0.09 |
| QAQLSPVQN | | | | |
| PLNGAVHLYA | 780 | 0.506 | 0.484 | 0.09 |
| QAQVGSVQT | | | | |
| PLNGTVHLYA | 781 | 0.405 | 0.483 | 0.14 |
| QAQTGWAQN | | | | |
| ALNGAVHLYA | 782 | 0.283 | 0.482 | 0.09 |
| QAQTGLVQT | | | | |
| PLNGAVHLYA | 783 | 0.562 | 0.482 | 35.42 |
| QAQSSMVQN | | | | |
| PLNGAVHLYA | 784 | 0.484 | 0.481 | 0.09 |
| KAQTAGVQN | | | | |
| PLNGAVHLYA | 785 | 0.516 | 0.480 | 0.09 |
| HAQTGWVQK | | | | |
| PLNGAVHLYA | 786 | 0.502 | 0.479 | 0.23 |
| QAQTSPVQK | | | | |
| PLNGAVHLYA | 787 | 0.841 | 0.477 | 7.66 |
| QAQAVGVQN | | | | |
| PLNGAVPLYA | 788 | 0.438 | 0.477 | 0.09 |
| QAQTGWGQN | | | | |
| PLNGTVHLYA | 789 | 0.495 | 0.477 | 0.09 |
| QAQLGSVQN | | | | |
| QLNGAVHLYA | 790 | 0.645 | 0.476 | 0.75 |
| QAQTGWVQH | | | | |
| PLNGSVHLYA | 791 | 0.175 | 0.475 | 1.4 |
| QAQTGWVKN | | | | |
| PLNGAVHLYD | 792 | 0.812 | 0.475 | 0.09 |
| QAQVAPVQN | | | | |
| PLNGAVNLYA | 793 | 0.460 | 0.473 | 0.19 |
| QAQVSSVQN | | | | |
| PLNGAVHLYA | 794 | 0.646 | 0.472 | 0.09 |
| QAQTLPVQK | | | | |
| PLNGSVHLYA | 795 | 0.337 | 0.469 | 0.93 |
| QAQTASVQN | | | | |
| ALNGAVHLYA | 796 | 0.294 | 0.468 | 0.37 |
| QAQTGWGQN | | | | |
| PLNGAVHLYA | 797 | 0.286 | 0.468 | 7.48 |
| QAQTGWGQN | | | | |
| PLNGAAHLYA | 798 | 0.451 | 0.467 | 0.09 |
| QAQTSPVQN | | | | |
| PLNGAVHLYA | 799 | 0.646 | 0.465 | 0.14 |
| QAQTGRVQH | | | | |
| PINGAVHLYA | 800 | 0.768 | 0.464 | 0.09 |
| QAQTGWVQS | | | | |
| PLNGAVHLYA | 801 | 0.454 | 0.464 | 2.24 |
| QAQTP SVQN | | | | |
| PLNGAVHLYD | 802 | 0.450 | 0.464 | 0.09 |
| QAQTTAVQN | | | | |
| PLNGNVHLYA | 803 | 0.415 | 0.462 | 0.09 |
| QAQTGWVQK | | | | |
| PLNGAVHLYA | 804 | 0.167 | 0.462 | 2.62 |
| QAQTGWVEN | | | | |
| PLNGAVNLYA | 805 | 0.371 | 0.461 | 0.19 |
| QAQTTPVQN | | | | |
| PLNGAVNLYA | 806 | 0.489 | 0.461 | 0.09 |
| QAQLSQVQN | | | | |
| SLNGAVHLYA | 807 | 0.263 | 0.460 | 0.14 |
| QAQVMSVQN | | | | |
| QLNGAVHLYA | 808 | 0.622 | 0.458 | 0.33 |
| QAQTGWVQS | | | | |
| PINGAVHLYA | 809 | 0.534 | 0.457 | 0.19 |
| QAQTGWFQN | | | | |
| PLNGDVHLYA | 810 | 0.419 | 0.456 | 0.14 |
| QAQVSSVQN | | | | |
| PLNGGVHLYA | 811 | 0.482 | 0.456 | 0.28 |
| QAQTGWVQS | | | | |
| PLNGAVNLYA | 812 | 0.559 | 0.456 | 0.09 |
| QAQRSTVQN | | | | |
| ALNGAVHLYA | 813 | 0.298 | 0.455 | 0.14 |
| QAQTGWVQY | | | | |
| PLNGAVHHYA | 814 | 0.170 | 0.455 | 0.09 |
| QAQTAAVQN | | | | |
| PLNGAVHRYA | 815 | 0.346 | 0.454 | 0.09 |
| QAQTGWVQH | | | | |
| QLNGAVHLYA | 816 | 0.627 | 0.453 | 0.37 |
| QAQTGWVHN | | | | |
| PLNGAVHLYA | 817 | 0.861 | 0.452 | 56.6 |
| QAQRGWVQN | | | | |
| PLNGAVHLYA | 818 | 0.233 | 0.450 | 0.14 |
| QAQTGSVEN | | | | |
| ALNGAVHLYA | 819 | 0.249 | 0.450 | 0.37 |
| QAQTGWVHN | | | | |
| PLNGAVNLYA | 820 | 0.494 | 0.449 | 0.09 |
| QAQTLAVQN | | | | |
| PLNGAVHLYA | 821 | 0.772 | 0.448 | 91.93 |
| QAQSSLVQN | | | | |
| ALNGAVHLYA | 822 | 0.203 | 0.448 | 0.37 |
| QAQTGWLQN | | | | |
| TLNGAVHLYA | 823 | 0.736 | 0.448 | 0.09 |
| QAQTLQVQN | | | | |
| PLNGAVHLYD | 824 | 0.439 | 0.448 | 0.09 |
| QAQVQSVQN | | | | |
| SLNGAVHLYA | 825 | 0.442 | 0.447 | 0.09 |
| QAQVTGVQN | | | | |
| QLNGAVHLYA | 826 | 0.849 | 0.444 | 0.09 |
| QAQSAVVQN | | | | |
| PLNGVVHLYA | 827 | 0.410 | 0.444 | 0.09 |
| QAQTGWVQT | | | | |
| PLNGAVHLYA | 828 | 0.373 | 0.444 | 0.61 |
| QAQTAWVKN | | | | |
| TLNGAVHLYA | 829 | 0.557 | 0.443 | 0.14 |
| QAQTLPVQN | | | | |
| ALNGAVHLYA | 830 | 0.683 | 0.442 | 0.09 |
| QAQTTGVQN | | | | |
| PLNGAVHLYA | 831 | 0.466 | 0.442 | 0.09 |
| KAQTMAVQN | | | | |
| PLNGAVHLYA | 832 | 0.636 | 0.442 | 10.61 |
| QAQTYSVQN | | | | |
| PLNGCVHLYA | 833 | 0.285 | 0.441 | 0.75 |
| QAQTGWVQN | | | | |
| PLNGAVHLYA | 834 | 0.491 | 0.440 | 0.09 |
| QAQLAAVQT | | | | |
| PLNGAVHLYA | 835 | 0.734 | 0.440 | 0.14 |
| QAQTALVKN | | | | |
| PLNGAVHLYA | 836 | 0.392 | 0.439 | 0.14 |
| KAQTVAVQN | | | | |
| PLNGAVHLYA | 837 | 0.225 | 0.439 | 0.37 |
| HAQTGWVQT | | | | |
| PLNGGVHLYA | 838 | 0.649 | 0.439 | 0.09 |
| QAQRGSVQN | | | | |
| PLNGAFHLYA | 839 | 0.462 | 0.436 | 0.14 |
| QAQTGGVQN | | | | |
| PLNGAVHLYA | 840 | 0.496 | 0.436 | 0.09 |
| LAQTSPVQN | | | | |
| PLNGAVHLYA | 841 | 0.225 | 0.435 | 2.9 |
| QAQTGWVQY | | | | |
| PLNGAVHLYA | 842 | 0.538 | 0.432 | 0.19 |
| QAQTLPVQT | | | | |
| PLNGAVHLYS | 843 | 0.499 | 0.432 | 0.23 |
| QAQTGWFQN | | | | |
| PLNGAVHLYA | 844 | 0.696 | 0.432 | 0.33 |
| QAQTGLVQH | | | | |
| PLNGAVHLYA | 845 | 0.628 | 0.432 | 0.98 |
| QAQTP TVQN | | | | |
| PLYGAVHLYA | 846 | 0.274 | 0.429 | 0.28 |
| QAQTAWVQN | | | | |
| PLNGAVHLYA | 847 | 0.275 | 0.428 | 0.23 |
| KAQTASVQN | | | | |
| PLNGAVHLYA | 848 | 0.285 | 0.428 | 0.19 |
| QAQTASVQK | | | | |
| PLDGSVHLYA | 849 | 0.641 | 0.424 | 0.09 |
| QAQTAWVQN | | | | |
| PLNGADHLYA | 850 | 0.540 | 0.423 | 0.09 |
| QAQTSSVQN | | | | |
| PLNGAVHLYA | 851 | 0.594 | 0.422 | 0.09 |
| QAQTSPVQI | | | | |
| PLNGAVHLYA | 852 | 0.661 | 0.420 | 40 |
| QAQIGWVQN | | | | |
| PLNGAVHLYA | 853 | 0.209 | 0.419 | 0.09 |
| KAQTSTVQN | | | | |
| PLNGAVHRYA | 854 | 0.237 | 0.419 | 0.19 |
| QAQTGWVQT | | | | |
| PLNGAVHLYD | 855 | 0.268 | 0.419 | 0.14 |
| QAQTSTVQN | | | | |
| QLNGAVHLYA | 856 | 0.568 | 0.418 | 69.31 |
| QAQTGLVQN | | | | |
| PLNGAVNLYA | 857 | 0.864 | 0.417 | 0.09 |
| QAQLGPVQN | | | | |
| PLNGAVHLYA | 858 | 0.400 | 0.417 | 0.23 |
| QAQTGWDKN | | | | |
| PLNGALHLYA | 859 | 0.432 | 0.416 | 0.14 |
| QAQTGWVKN | | | | |
| PLNGNVHLYA | 860 | 0.698 | 0.414 | 0.09 |
| HAQTGWVQN | | | | |
| PLNGAVHLYA | 861 | 0.861 | 0.414 | 8.23 |
| QAQKSSVQN | | | | |
| PLNGAVHLYA | 862 | 0.691 | 0.412 | 30.7 |
| QAQTGYVQN | | | | |
| PVNGAVHLYA | 863 | 0.456 | 0.412 | 0.09 |
| QAQVGSVQN | | | | |
| PLNGGVHLYA | 864 | 0.536 | 0.411 | 1.17 |
| QAQTGWVKN | | | | |
| PLNGAVHLYA | 865 | 0.627 | 0.410 | 0.09 |
| QAQRGWVQT | | | | |
| PLNGAVNLYA | 866 | 0.729 | 0.410 | 0.09 |
| QAQRSLVQN | | | | |
| PINGAVHLYA | 867 | 0.358 | 0.410 | 0.51 |
| QAQTGWDQN | | | | |
| PLNGNVHLYA | 868 | 0.536 | 0.410 | 0.23 |
| QAQTGWVQT | | | | |
| PLNGAVHLYA | 869 | 0.265 | 0.409 | 0.23 |
| KAQTGWFQN | | | | |
| PLNGAVHLYA | 870 | 0.421 | 0.408 | 0.09 |
| QEQTSTVQN | | | | |
| PLNGAVHLYA | 871 | 0.281 | 0.406 | 0.14 |
| QAQTGGVEN | | | | |
| PLNGAVHLYS | 872 | 0.831 | 0.406 | 0.09 |
| QAQTGWVQI | | | | |
| SLNGAVHLYA | 873 | 0.480 | 0.406 | 0.09 |
| QAQTGLVQK | | | | |
| PLNGAVHLYA | 874 | 0.605 | 0.403 | 0.09 |
| QAQTGCVRN | | | | |
| PLNGADHLYA | 875 | 0.758 | 0.402 | 0.09 |
| QAQVTPVQN | | | | |
| PLNGAVHLYA | 876 | 0.129 | 0.400 | 0.28 |
| PAQTGWVQT | | | | |
| PLNGDVHLYA | 877 | 0.900 | 0.400 | 0.09 |
| QAQTSAVQN | | | | |
| TLNGAVHLYA | 878 | 0.635 | 0.399 | 0.09 |
| QAQSSLVQN | | | | |
| SLNGAVHLYA | 879 | 0.645 | 0.399 | 0.14 |
| QAQRSVVQN | | | | |
| SLNGAVHLYA | 880 | 0.817 | 0.397 | 0.33 |
| QAQTGWVQS | | | | |
| PVNGAVHLYA | 881 | 0.800 | 0.397 | 0.14 |
| QAQTGWVQS | | | | |
| PLNGAVHLYA | 882 | 0.619 | 0.396 | 0.09 |
| QAQLSPVQS | | | | |
| PLNGAVHLYA | 883 | 0.285 | 0.395 | 0.09 |
| QPQTGWLQN | | | | |
| PLNGAVNLYA | 884 | 0.719 | 0.394 | 0.14 |
| QAQSAPVQN | | | | |
| PLNGTVHLYA | 885 | 0.384 | 0.394 | 0.09 |
| QAQTGWVPN | | | | |
| PVDGAVHLYA | 886 | 0.792 | 0.394 | 0.23 |
| QAQTGSVQN | | | | |
| PLNGAVHLYD | 887 | 0.261 | 0.393 | 0.09 |
| QAQTAAVQN | | | | |
| PLKGAVHLYA | 888 | 0.352 | 0.392 | 0.09 |
| QAQTSTVQN | | | | |
| PLNGAVHLYA | 889 | 0.730 | 0.392 | 0.14 |
| QAQTGPVQT | | | | |
| PLNGAVHLYA | 890 | 0.432 | 0.392 | 0.14 |
| QAQTVGVQK | | | | |
| PLNGAVNLYA | 891 | 0.451 | 0.391 | 0.14 |
| QAQTSTVQN | | | | |
| PLNGAVHLYA | 892 | 0.414 | 0.390 | 0.09 |
| RAQTVSVQN | | | | |
| PLNGAVHLYA | 893 | 0.431 | 0.390 | 0.09 |
| QAQTASGQN | | | | |
| PLNGAVHLYA | 894 | 0.631 | 0.390 | 20.28 |
| QAQSVSVQN | | | | |
| PLNGAVHLYA | 895 | 0.724 | 0.389 | 0.09 |
| QAQSGPVQT | | | | |
| QLNGAVHLYA | 896 | 0.578 | 0.389 | 0.47 |
| QAQTGWGQN | | | | |
| PLNGAVHLYA | 897 | 0.670 | 0.386 | 0.09 |
| QAQVMSVKN | | | | |
| PLNGTVHLYA | 898 | 0.557 | 0.386 | 0.09 |
| KAQTGSVQN | | | | |
| PLNGAVNLYA | 899 | 0.527 | 0.385 | 0.09 |
| QAQTSLVQN | | | | |
| PINGAVHLYA | 900 | 0.867 | 0.385 | 0.14 |
| QAQTLSVQN | | | | |
| PLNGAVHLYA | 901 | 0.458 | 0.384 | 0.09 |
| QAQLGSVQK | | | | |
| TLNGAVHLYA | 902 | 0.728 | 0.383 | 0.09 |
| QAQTAMVQN | | | | |
| PLNGAVHLYA | 903 | 0.775 | 0.383 | 0.09 |
| QAHLSSVQN | | | | |
| PLNGAVHLYA | 904 | 0.552 | 0.380 | 0.09 |
| KAQVMSVQN | | | | |
| PLNGAVHLYA | 905 | 0.569 | 0.380 | 0.09 |
| QAQTGLVLN | | | | |
| PLNGGVHLYA | 906 | 0.491 | 0.378 | 1.03 |
| QAQTGWVQK | | | | |
| PLNGAVHLYA | 907 | 0.250 | 0.378 | 0.61 |
| KAQTGWVQK | | | | |
| PLNGAVLLYA | 908 | 0.482 | 0.378 | 0.09 |
| QAQTASVQN | | | | |
| PLNGDVHLYA | 909 | 0.507 | 0.378 | 0.09 |
| QAQTMAVQN | | | | |
| PLNGAVHLYA | 910 | 0.258 | 0.377 | 0.09 |
| KAQTAAVQN | | | | |
| PLNGAVHLYA | 911 | 0.549 | 0.377 | 763.5 |
| QAQTGLVQN | | | | |
| PLNGDVHLYA | 912 | 0.699 | 0.376 | 0.14 |
| QAQSSPVQN | | | | |
| PLNGAVHLYA | 913 | 0.832 | 0.376 | 14.96 |
| QAQTSNVQN | | | | |
| ALDGAVHLYA | 914 | 0.702 | 0.376 | 0.47 |
| QAQTGSVQN | | | | |
| PLNGAVHLYS | 915 | 0.848 | 0.375 | 0.09 |
| QAQTSSVQN | | | | |
| PLKGAVHLYA | 916 | 0.390 | 0.373 | 0.19 |
| QAQLSPVQN | | | | |
| LLNGAVHLYA | 917 | 0.349 | 0.373 | 0.09 |
| QAQTVSVQN | | | | |
| PLNGGVHLYA | 918 | 0.571 | 0.373 | 0.37 |
| QAQTGWVQI | | | | |
| PLNGAVHLYS | 919 | 0.863 | 0.372 | 0.14 |
| QAQTGWVRN | | | | |
| PLNGAVHLYA | 920 | 0.426 | 0.371 | 0.09 |
| QAQTGWGHN | | | | |
| PHNGAVHLYA | 921 | 0.849 | 0.371 | 0.09 |
| QAQTTSVQN | | | | |
| PLNRAVHLYA | 922 | 0.436 | 0.371 | 0.09 |
| QAQTGWVQK | | | | |
| PLNGAVHLYD | 923 | 0.509 | 0.371 | 0.09 |
| QAQRASVQN | | | | |
| PLNGAVHLYS | 924 | 0.800 | 0.370 | 584.7 |
| QAQTGWVQN | | | | |
| PLNGSVHLYA | 925 | 0.401 | 0.370 | 0.42 |
| QAQTGTVQN | | | | |
| PLNGAVHLYD | 926 | 0.752 | 0.370 | 0.09 |
| QAQTSGVQN | | | | |
| PLNGAVHLYA | 927 | 0.389 | 0.368 | 0.19 |
| QAKTGWFQN | | | | |
| KLDGAVHLYA | 928 | 0.763 | 0.367 | 0.14 |
| QAQTGLVQN | | | | |
| PLNGAVHLYA | 929 | 0.581 | 0.363 | 9.39 |
| QAQSQMVQN | | | | |
| TLNGAVHLYA | 930 | 0.706 | 0.363 | 0.09 |
| QAQTSVVQN | | | | |
| PLNGAVHLYA | 931 | 0.449 | 0.361 | 0.09 |
| KAQTGTVQN | | | | |
| PLNGAVHLYA | 932 | 0.440 | 0.361 | 0.09 |
| QAQTVSVHN | | | | |
| PLNGAVHLYA | 933 | 0.559 | 0.361 | 0.19 |
| QAQLSSVQT | | | | |
| PVNGAVHLYA | 934 | 0.791 | 0.359 | 0.28 |
| QAQTGWFQN | | | | |
| PLNGAVHLYA | 935 | 0.266 | 0.356 | 0.09 |
| QAQTASVRN | | | | |
| PLNGDVHLYA | 936 | 0.410 | 0.355 | 0.09 |
| QAQTSTVQN | | | | |
| PLNGAVHLYA | 937 | 0.477 | 0.354 | 0.09 |
| KAQLGSVQN | | | | |
| PINGAVHLYA | 938 | 0.774 | 0.354 | 0.09 |
| QAQLVPVQN | | | | |
| PLNGAVHHYA | 939 | 0.833 | 0.354 | 0.09 |
| QAQLGPVQN | | | | |
| PLNGAVHLYA | 940 | 0.387 | 0.353 | 0.19 |
| PAQTGWVQH | | | | |
| PLNGAVHLYA | 941 | 0.696 | 0.353 | 0.56 |
| QAQVPAVQN | | | | |
| PLNGAVNLYA | 942 | 0.774 | 0.353 | 0.09 |
| QAQRLSVQN | | | | |
| PLNGAVHLYA | 943 | 0.534 | 0.352 | 0.23 |
| QAQTGRVQK | | | | |
| PLNGAVLLYA | 944 | 0.370 | 0.352 | 0.09 |
| QAQTVSVQN | | | | |
| PLNGAVHLYA | 945 | 0.510 | 0.351 | 11.4 |
| QAQAMSVQN | | | | |
| PLNGAVHLYA | 946 | 0.451 | 0.351 | 0.23 |
| QAQTAWVHN | | | | |
| PLNGAVHLYA | 947 | 0.615 | 0.350 | 0.09 |
| QPQTGWVRN | | | | |
| PLNGAVHLYA | 948 | 0.548 | 0.350 | 0.09 |
| QEQTGWLQN | | | | |
| PLNGAVHLYA | 949 | 0.535 | 0.348 | 0.09 |
| KAQTSAVQN | | | | |
| PLNGSVHLYA | 950 | 0.126 | 0.348 | 0.98 |
| QAQTVWVQN | | | | |
| PLNGTVHLYA | 951 | 0.330 | 0.347 | 0.33 |
| QAQTGWFQN | | | | |
| PLNGAVNLYA | 952 | 0.375 | 0.347 | 0.23 |
| QAQVASVQN | | | | |
| PLNGAVHLYA | 953 | 0.674 | 0.347 | 0.19 |
| QAQTGLVRN | | | | |
| PLNGAVNLYA | 954 | 0.587 | 0.346 | 0.09 |
| QAQLSTVQN | | | | |
| PLNGAVHLYA | 955 | 0.721 | 0.346 | 0.09 |
| KAQTQPVQN | | | | |
| PLNGTVHLYA | 956 | 0.382 | 0.346 | 0.33 |
| QAQTGWGQN | | | | |
| PLNGAVHLYA | 957 | 0.453 | 0.345 | 0.09 |
| QAQRTLVQT | | | | |
| PLNGAVHLYA | 958 | 0.157 | 0.345 | 0.61 |
| QAQTGSIQN | | | | |
| QLNGAVHLYA | 959 | 0.574 | 0.344 | 1.4 |
| QAQTGWDQN | | | | |
| PLNGAVHLYA | 960 | 0.599 | 0.344 | 0.14 |
| QAQLSSVKN | | | | |
| QLNGAVHLYA | 961 | 0.451 | 0.343 | 1.45 |
| QAQTVSVQN | | | | |
| PLNGAVHLYA | 962 | 0.726 | 0.343 | 0.09 |
| QAQTLQVQK | | | | |
| TLNGAVHLYA | 963 | 0.573 | 0.343 | 0.14 |
| QAQVLPVQN | | | | |
| PLNGAVHLYA | 964 | 0.756 | 0.342 | 0.09 |
| QAQVGSVKN | | | | |
| PLNGAVHLYA | 965 | 0.744 | 0.341 | 0.09 |
| QAQLAPLQN | | | | |
| QLNGAVHLYA | 966 | 0.716 | 0.341 | 0.09 |
| QAQTGSVKN | | | | |
| PLNGAVHHYA | 967 | 0.620 | 0.341 | 0.09 |
| QAQRSTVQN | | | | |
| QLNGSVHLYA | 968 | 0.266 | 0.341 | 0.23 |
| QAQTGRVQN | | | | |
| TLNGAVHLYA | 969 | 0.818 | 0.340 | 0.09 |
| QAQVKPVQN | | | | |
| PLNGTVHLYA | 970 | 0.784 | 0.339 | 24.58 |
| QAQTGLVQN | | | | |
| PLNGAVHLYA | 971 | 0.396 | 0.338 | 0.65 |
| QAQTPGVQN | | | | |
| PINGAVHLYA | 972 | 0.713 | 0.338 | 0.28 |
| QAQTSSVQN | | | | |
| PLNGAVHLYA | 973 | 0.428 | 0.337 | 0.09 |
| QAQLSAVQT | | | | |
| PLNGAVHLYS | 974 | 0.446 | 0.337 | 0.47 |
| QAQTGWDQN | | | | |
| PLNCAVHLYA | 975 | 0.856 | 0.336 | 14.39 |
| QAQTGWVQN | | | | |
| PLNGAVNLYA | 976 | 0.624 | 0.336 | 0.14 |
| QAQTMPVQN | | | | |
| PLNGAVHLYS | 977 | 0.283 | 0.335 | 0.09 |
| QAQTVAVQN | | | | |
| PLNGTVHLYA | 978 | 0.366 | 0.334 | 0.79 |
| QAQTGWVKN | | | | |
| PLNGGVHLYA | 979 | 0.465 | 0.333 | 0.37 |
| QAQTGWVHN | | | | |
| QLNGAVHLYA | 980 | 0.558 | 0.332 | 1.59 |
| QAQTGWVQK | | | | |
| PLNGAVQLYA | 981 | 0.524 | 0.331 | 0.09 |
| QAQTLAVQN | | | | |
| PLNGTVHLYA | 982 | 0.695 | 0.331 | 0.09 |
| QAQVGSVQN | | | | |
| PLNGGVHLYA | 983 | 0.764 | 0.330 | 17.67 |
| QAQTGGVQN | | | | |
| PLKGAVHLYA | 984 | 0.474 | 0.330 | 0.09 |
| QAQVSQVQN | | | | |
| PLNGAVHHYA | 985 | 0.526 | 0.330 | 0.09 |
| QAQTQPVQN | | | | |
| PLNGAVHLYA | 986 | 0.156 | 0.329 | 0.33 |
| QAP TGWVQT | | | | |
| PLNGAVNLYA | 987 | 0.343 | 0.329 | 0.33 |
| QAQTMSVQN | | | | |
| PLNGAVHLYA | 988 | 0.681 | 0.329 | 29.16 |
| QAQTGVVQN | | | | |
| PLNGAVHLYA | 989 | 0.622 | 0.328 | 0.42 |
| QAQVPQVQN | | | | |
| PHNGAVHLYA | 990 | 0.295 | 0.327 | 0.14 |
| QAQTMSVQN | | | | |
| QLNGAVHLYA | 991 | 0.493 | 0.327 | 0.56 |
| QAQTGWLQN | | | | |
| PLNGAVHLYA | 992 | 0.118 | 0.327 | 1.03 |
| QAQTGCVQK | | | | |
| SLNGAVHLYA | 993 | 0.712 | 0.327 | 0.28 |
| QAQTSLVQN | | | | |
| PLNGAVHLYA | 994 | 0.422 | 0.326 | 0.23 |
| QAQTRWVQT | | | | |
| TLNGAVHLYA | 995 | 0.743 | 0.325 | 0.09 |
| QAQTVQVQN | | | | |
| ALNGAVHLYA | 996 | 0.762 | 0.324 | 0.09 |
| QAQKGGVQN | | | | |
| PLNGAVHLYD | 997 | 0.143 | 0.323 | 0.09 |
| QAQVSGVQN | | | | |
| PLNGAVHLYA | 998 | 0.554 | 0.323 | 0.65 |
| QAQTGLDQN | | | | |
| PLNGAVPLYA | 999 | 0.387 | 0.323 | 0.42 |
| QAQTGWVQT | | | | |
| PLNGAVHLYA | 1000 | 0.414 | 0.323 | 0.09 |
| QAQVS SVHN | | | | |
| TLNGAVHLYA | 1001 | 0.264 | 0.322 | 0.09 |
| QAQTNSVQN | | | | |
| PLNGAGHLYA | 1002 | 0.479 | 0.321 | 0.09 |
| QAQLSPVQN | | | | |
| HLNGAVHLYA | 1003 | 0.532 | 0.321 | 0.19 |
| QAQTGRVQN | | | | |
| PLNGAVHLYD | 1004 | 0.872 | 0.320 | 0.09 |
| QAQTAMVQN | | | | |
| PINGAVHLYA | 1005 | 0.646 | 0.320 | 0.33 |
| QAQTGWVQH | | | | |
| PLNGAVQLYA | 1006 | 0.479 | 0.319 | 0.09 |
| QAQTLQVQN | | | | |
| PLNGAVHLYA | 1007 | 0.323 | 0.318 | 0.28 |
| QAQKGWVQT | | | | |
| PLNGAVHHYA | 1008 | 0.592 | 0.318 | 0.09 |
| QAQSTSVQN | | | | |
| PLNGYVHLYA | 1009 | 0.187 | 0.317 | 2.99 |
| QAQTGWVQN | | | | |
| PLNGAVHIYA | 1010 | 0.599 | 0.316 | 0.09 |
| QAQVQSVQN | | | | |
| PLIGAVHLYA | 1011 | 0.535 | 0.315 | 0.09 |
| QAQLSPVQN | | | | |
| TLNGAVHLYA | 1012 | 0.774 | 0.315 | 0.09 |
| QAQLLPVQN | | | | |
| PLNGAVHLYS | 1013 | 0.747 | 0.315 | 0.84 |
| QAQTGWVQT | | | | |
| ALNGAVHLYA | 1014 | 0.444 | 0.315 | 0.14 |
| QAQTQLVQN | | | | |
| PLNGAVHLYA | 1015 | 0.810 | 0.315 | 34.86 |
| QAQSLPVQN | | | | |
| PVNGAVHLYA | 1016 | 0.890 | 0.314 | 0.09 |
| QAQTGWVPN | | | | |
| PLNGAVHLYA | 1017 | 0.247 | 0.313 | 0.19 |
| QAKTVSVQN | | | | |
| PLNGAVHLYA | 1018 | 0.645 | 0.313 | 0.09 |
| QAQTTSVHN | | | | |
| PVNGAVHLYA | 1019 | 0.765 | 0.313 | 939 |
| QAQTGWVQN | | | | |
| PLNGAVHLYA | 1020 | 0.476 | 0.313 | 0.09 |
| QAHTGWVQI | | | | |
| PLNGAVRLYA | 1021 | 0.611 | 0.313 | 0.09 |
| QAQTASVQN | | | | |
| PLNGGVHLYA | 1022 | 0.646 | 0.313 | 0.09 |
| QAQTGLVQK | | | | |
| PLNGAVNLYA | 1023 | 0.652 | 0.313 | 0.14 |
| QAQVMSVQN | | | | |
| PLNGAVNLYA | 1024 | 0.682 | 0.312 | 0.09 |
| QAQSMSVQN | | | | |
| PLNGAVHLYA | 1025 | 0.513 | 0.312 | 0.09 |
| PAQTGWGQN | | | | |
| QLNGAVHLYA | 1026 | 0.595 | 0.311 | 0.47 |
| QAQTGWAQN | | | | |
| PLNGAVHLYA | 1027 | 0.451 | 0.311 | 23.13 |
| QAQTVWVQN | | | | |
| QLNGAVHLYA | 1028 | 0.742 | 0.311 | 0.09 |
| QAQSVSVQN | | | | |
| PLNGAVHLYA | 1029 | 0.426 | 0.311 | 0.09 |
| QAQTAQLQN | | | | |
| PLNGAVHLYA | 1030 | 0.453 | 0.311 | 0.09 |
| QAHVASVQN | | | | |
| PLNGAVHLYA | 1031 | 0.440 | 0.310 | 0.09 |
| KAQRSTVQN | | | | |
| QLNGAVHLYA | 1032 | 0.509 | 0.310 | 1.92 |
| QAQTGWVKN | | | | |
| PLNGAVHLYA | 1033 | 0.281 | 0.310 | 0.28 |
| QAQTRWVKN | | | | |
| PLNGAVHLYA | 1034 | 0.381 | 0.310 | 0.19 |
| QAQTAWIQN | | | | |
| PLNGAVHLYA | 1035 | 0.393 | 0.310 | 0.09 |
| QAQLSQVKN | | | | |
| SLNGAVHLYA | 1036 | 0.816 | 0.310 | 2284 |
| QAQTGWVQN | | | | |
| PLNGAVHLYA | 1037 | 0.390 | 0.309 | 0.14 |
| QAQTSTVKN | | | | |
| PLNGAVHLYA | 1038 | 0.654 | 0.309 | 16.82 |
| QAQALPVQN | | | | |
| PLNGAVHLYD | 1039 | 0.158 | 0.309 | 0.33 |
| QAQTGWFQN | | | | |
| PLNGAVHVYA | 1040 | 0.527 | 0.309 | 0.09 |
| QAQTGWFQN | | | | |
| PINGAVHLYA | 1041 | 0.386 | 0.308 | 0.09 |
| QAQVGSVQN | | | | |
| PLNGSVHLYA | 1042 | 0.418 | 0.308 | 0.14 |
| QAQTTGVQN | | | | |
| PLNGAVHLYA | 1043 | 0.874 | 0.307 | 0.09 |
| KAQASPVQN | | | | |
| PLNGSVHLYA | 1044 | 0.192 | 0.307 | 0.09 |
| QAQKAWVQN | | | | |
| SLNGAVHLYA | 1045 | 0.270 | 0.307 | 0.09 |
| QAQVLGVQN | | | | |
| PLNGAVHLYA | 1046 | 0.827 | 0.306 | 0.09 |
| QEQLMPVQN | | | | |
| ALNGAVHLYA | 1047 | 0.872 | 0.306 | 18.51 |
| QSQTGWVQN | | | | |
| PLNGYVHLYD | 1048 | 0.508 | 0.305 | 0.09 |
| QAQTGWVQN | | | | |
| PLNGAVHLYA | 1049 | 0.505 | 0.305 | 0.09 |
| QAQTSMVQT | | | | |
| QLNGAVHLYA | 1050 | 0.588 | 0.304 | 0.14 |
| QAQISSVQN | | | | |
| PVNGAVHLYA | 1051 | 0.723 | 0.304 | 1.12 |
| QAQTGWVQT | | | | |
| PLNGAVHLYA | 1052 | 0.713 | 0.303 | 0.09 |
| QEQTATVQN | | | | |
| PLNGAVYLYA | 1053 | 0.352 | 0.302 | 0.09 |
| QAQTVSVQN | | | | |
| PLNGNVHLYA | 1054 | 0.521 | 0.302 | 0.09 |
| QAQTASVQN | | | | |
| PLNGYVHLYA | 1055 | 0.556 | 0.301 | 0.09 |
| QAQTGLVQN | | | | |
| PLNGAVHLYD | 1056 | 0.402 | 0.301 | 0.14 |
| QAQTLSVQN | | | | |
| PLNGAVHLYA | 1057 | 0.499 | 0.300 | 0.14 |
| QAQTSSVQH | | | | |
| SLNGAVHLYA | 1058 | 0.737 | 0.300 | 0.19 |
| QAQTGWVPN | | | | |
| PLNGAVHLYA | 1059 | 0.839 | 0.300 | 0.09 |
| QAQTAMVKN | | | | |
| TLNGAVHLYA | 1060 | 0.700 | 0.299 | 0.14 |
| QAQRLSVQN | | | | |
| ALNGAVHLYA | 1061 | 0.341 | 0.297 | 1.64 |
| QAQTGWVQK | | | | |
| PLNGAVHLYA | 1062 | 0.722 | 0.297 | 0.19 |
| KAQTALVQN | | | | |
| QLNGAVHLYA | 1063 | 0.743 | 0.297 | 0.23 |
| QAQTAKVQN | | | | |
| PLNGADHLYA | 1064 | 0.744 | 0.297 | 0.14 |
| QAQTTSVQN | | | | |
| PLNGAVHLYA | 1065 | 0.259 | 0.297 | 0.93 |
| QAQTPWVQN | | | | |
| PLNGTVHLYA | 1066 | 0.324 | 0.296 | 0.28 |
| QAQTGWIQN | | | | |
| PLNGAVHLYA | 1067 | 0.286 | 0.296 | 0.23 |
| QAHTGWVQH | | | | |
| PLNGAVHLYA | 1068 | 0.622 | 0.296 | 0.09 |
| RAQTASVQN | | | | |
| TLNGAVHLYA | 1069 | 0.876 | 0.295 | 0.14 |
| QAQRSLVQN | | | | |
| PLNGAVHLYA | 1070 | 0.481 | 0.294 | 0.14 |
| QAHTGWVQK | | | | |
| PLNGDVHLYA | 1071 | 0.859 | 0.294 | 0.14 |
| QAQTAQVQN | | | | |
| TLNGAVHLYA | 1072 | 0.407 | 0.294 | 0.14 |
| QAQTQPVQN | | | | |
| PLNGAVHLYA | 1073 | 0.505 | 0.294 | 0.7 |
| QAQTPRVQN | | | | |
| PRNGAVHLYA | 1074 | 0.200 | 0.293 | 0.09 |
| QAQTMSVQN | | | | |
| PLNGAVHLYA | 1075 | 0.118 | 0.293 | 0.23 |
| QAHTVSVQN | | | | |
| PVNGAVHLYA | 1076 | 0.823 | 0.293 | 0.14 |
| QAQTGLVQT | | | | |
| PLNGAVHLYA | 1077 | 0.899 | 0.292 | 87.25 |
| QAQSSSVQN | | | | |
| PLNGAVHLYA | 1078 | 0.497 | 0.292 | 0.84 |
| QAQRPPVQN | | | | |
| PLNGAVHLYT | 1079 | 0.779 | 0.291 | 0.09 |
| QAQVSPVQN | | | | |
| PLNGAVPLYA | 1080 | 0.661 | 0.291 | 0.09 |
| QAQTTPVQN | | | | |
| ALNGAVHLYA | 1081 | 0.293 | 0.290 | 1.59 |
| QAQTGWVKN | | | | |
| SLNGAVHLYA | 1082 | 0.871 | 0.290 | 0.56 |
| QAQTGWVRN | | | | |
| PLNGAVHLYA | 1083 | 0.316 | 0.288 | 0.09 |
| QAQLAGVKN | | | | |
| PLNGAVHLYA | 1084 | 0.427 | 0.288 | 0.09 |
| QAQTSPAQN | | | | |
| PLNGAVHLYA | 1085 | 0.539 | 0.288 | 0.19 |
| KAQLSSVQN | | | | |
| PLNGAVHLYA | 1086 | 0.526 | 0.285 | 0.14 |
| QAQRSPVQT | | | | |
| PLNGAVHLYA | 1087 | 0.826 | 0.285 | 0.14 |
| HAQTMSVQN | | | | |
| PLNGNVHLYA | 1088 | 0.429 | 0.284 | 0.14 |
| QAQTGWVKN | | | | |
| PLNGAVHLYA | 1089 | 0.409 | 0.284 | 0.09 |
| QAQTGWVPH | | | | |
| PLNGAVHLYA | 1090 | 0.442 | 0.284 | 0.09 |
| QAQPGWGQN | | | | |
| PLNGVVHLYA | 1091 | 0.291 | 0.284 | 0.14 |
| QAQTVSVQN | | | | |
| PLNGDVHLYA | 1092 | 0.499 | 0.283 | 0.19 |
| QAQTSSVQN | | | | |
| PLNGAVHIYA | 1093 | 0.570 | 0.283 | 0.14 |
| QAQTLSVQN | | | | |
| PLNGAVHLYA | 1094 | 0.597 | 0.283 | 0.19 |
| QAQIPPVQN | | | | |
| SLNGAVHLYA | 1095 | 0.856 | 0.282 | 1.03 |
| QAQTGWVQH | | | | |
| PLNAAVHLYA | 1096 | 0.466 | 0.282 | 0.14 |
| QAQTGWDQN | | | | |
| PLNGAVHRYA | 1097 | 0.823 | 0.282 | 0.09 |
| QAQTTSVQN | | | | |
| PLNGSVHLYA | 1098 | 0.394 | 0.281 | 0.09 |
| QAQPGSVQN | | | | |
| PLDGAVHLYA | 1099 | 0.838 | 0.281 | 0.09 |
| QAQTGWVEN | | | | |
| PLNGAVHLYA | 1100 | 0.524 | 0.281 | 0.33 |
| QAQTGRVKN | | | | |
| PLNGAVHLYA | 1101 | 0.435 | 0.281 | 0.33 |
| QAQTGRLQN | | | | |
| PLNGSVHLYA | 1102 | 0.584 | 0.281 | 0.28 |
| QAQTARVQN | | | | |
| PLNGAVHLYD | 1103 | 0.378 | 0.280 | 0.19 |
| QAQLSSVQN | | | | |
| ALNGAVHLYA | 1104 | 0.550 | 0.280 | 0.09 |
| QAQTRLVQN | | | | |
| PLNGAVNLYA | 1105 | 0.780 | 0.280 | 0.19 |
| QAQTAQVQN | | | | |
| PLNGAVHLYA | 1106 | 0.461 | 0.279 | 0.09 |
| QAQLSSVQH | | | | |
| PLNGAVHLYA | 1107 | 0.799 | 0.279 | 0.09 |
| QAHTGPVQN | | | | |
| PLKGAVHLYA | 1108 | 0.872 | 0.279 | 0.09 |
| QAQTLSVQN | | | | |
| ALNGAVHLYA | 1109 | 0.499 | 0.279 | 0.09 |
| QAQKAWVQN | | | | |
| PLNGAVHLYA | 1110 | 0.386 | 0.278 | 0.09 |
| QAQAGWVQT | | | | |
| PLNGAVHLYA | 1111 | 0.709 | 0.278 | 0.19 |
| QAQTGLVQS | | | | |
| PLNGAVHLYA | 1112 | 0.436 | 0.277 | 0.14 |
| QAQTGCVQI | | | | |
| QLNGAVHLYA | 1113 | 0.529 | 0.276 | 1.26 |
| QAQPGWVQN | | | | |
| PLNGAVHLYA | 1114 | 0.678 | 0.276 | 0.37 |
| QAQRPGVQN | | | | |
| PLNGAVHLYA | 1115 | 0.265 | 0.276 | 0.09 |
| QAQTAAVQH | | | | |
| PLNGADHLYA | 1116 | 0.728 | 0.276 | 0.09 |
| QAQTMPVQN | | | | |
| PLNGAVHLYA | 1117 | 0.198 | 0.276 | 0.33 |
| KAQTGWVQT | | | | |
| QLNGAVHLYA | 1118 | 0.819 | 0.275 | 0.09 |
| QAQKAWVQN | | | | |
| PLNGAVHLYA | 1119 | 0.116 | 0.274 | 0.98 |
| QAQTGCDQN | | | | |
| PLNGTVHLYA | 1120 | 0.513 | 0.274 | 0.09 |
| QEQTGSVQN | | | | |
| PLNGAVNLYA | 1121 | 0.671 | 0.274 | 0.09 |
| QAQSSLVQN | | | | |
| ALNGAVHLYA | 1122 | 0.296 | 0.274 | 0.23 |
| QAQTGWVEN | | | | |
| PLNGTVHLYA | 1123 | 0.405 | 0.274 | 0.14 |
| QAQTGWVQY | | | | |
| PINGAVHLYA | 1124 | 0.373 | 0.273 | 0.75 |
| QAQTGWVQK | | | | |
| PLNGAVHLYA | 1125 | 0.820 | 0.273 | 0.09 |
| QAQTGRVRN | | | | |
| PLNGAVHLYA | 1126 | 0.590 | 0.272 | 0.37 |
| QAQTGRDQN | | | | |
| QLNGAVHLYA | 1127 | 0.409 | 0.272 | 0.23 |
| QAQSALVQN | | | | |
| PLNGVVHLYA | 1128 | 0.422 | 0.272 | 0.09 |
| QAQTGWVQK | | | | |
| PLNGAVHLYA | 1129 | 0.534 | 0.272 | 0.14 |
| QAHTGWGQN | | | | |
| PLNGDVHLYA | 1130 | 0.638 | 0.272 | 0.09 |
| QAQRSLVQN | | | | |
| PLNGNVHLYA | 1131 | 0.711 | 0.272 | 0.09 |
| QAQTVWVQN | | | | |
| PLNGAVHLYA | 1132 | 0.332 | 0.272 | 0.23 |
| QAP TGWVQH | | | | |
| PLNGAVHLYS | 1133 | 0.725 | 0.271 | 0.23 |
| QAQTGWGQN | | | | |
| ALNGSVHLYA | 1134 | 0.729 | 0.271 | 0.09 |
| QAQTGWVQT | | | | |
| PLNGAVQLYA | 1135 | 0.347 | 0.271 | 0.09 |
| QAQRSTVQN | | | | |
| PLNGTVHLYA | 1136 | 0.434 | 0.271 | 0.28 |
| QAQTGTVQN | | | | |
| PLNGTVHLYA | 1137 | 0.549 | 0.271 | 0.37 |
| QAQTGWVQI | | | | |
| QLNGYVHLYA | 1138 | 0.513 | 0.626 | 0.09 |
| QAQTGWVQN | | | | |

TABLE 24
NGS fold-enrichment of TTD-001 matured AAV capsid variants brain and DRG of
cynomolgus macaques and the brain of marmosets

| | | Brain of | | DRG of | | | |
|---|---|---|---|---|---|---|---|
| | | cynomolgus | | cynomolgus | | Brain of | |
| | | macaques | | macaques | | marmosets | |
| | | Fold | Fold | Fold | Fold | Fold | Fold |
| | | change | change | change | change | change | change |
| | SEQ | relative | relative | relative | relative | relative | relative |
| | ID | to | to | to | to | to | to |
| Sequence | NO | AAV9 | TTD-001 | AAV9 | TTD-001 | AAV9 | TTD-001 |
| ALNGAVHLYAQAQTG | 1122 | 12.707 | 0.274 | 0.000 | 0.000 | 420.579 | 571.155 |
| WVEN | | | | | | | |
| PINGAVHLYAQAQTG | 40 | 8.716 | 0.188 | 2.100 | 13.718 | 573.779 | 779.203 |
| WVEN | | | | | | | |
| PLNGAVHLNAQAQTG | 41 | 0.919 | 0.020 | 2.175 | 14.212 | 677.878 | 920.572 |
| WVEN | | | | | | | |
| PLNGAVHLYAQAQSG | 42 | 5.505 | 0.119 | 0.000 | 0.000 | 263.098 | 357.292 |
| WVEN | | | | | | | |
| PLNGAVHLYAQAQTA | 43 | 8.721 | 0.188 | 0.827 | 5.405 | 236.026 | 320.528 |
| WVEN | | | | | | | |
| PLNGAVHLYAQAQTG | 44 | 4.126 | 0.089 | 0.245 | 1.603 | 475.164 | 645.282 |
| CVEN | | | | | | | |
| PLNGAVHLYAQAQTG | 871 | 18.851 | 0.406 | 0.491 | 3.206 | 288.900 | 392.332 |
| GVEN | | | | | | | |
| PLNGAVHLYAQAQTG | 45 | 5.050 | 0.109 | 0.000 | 0.000 | 541.666 | 735.593 |
| LVEN | | | | | | | |
| PLNGAVHLYAQAQTG | 818 | 20.878 | 0.450 | 2.856 | 18.662 | 571.096 | 775.559 |
| SVEN | | | | | | | |
| PLNGAVHLYAQAQTG | 804 | 21.437 | 0.462 | 1.362 | 8.901 | 381.679 | 518.327 |
| WVEN | | | | | | | |
| PLNGSVHLYAQAQTG | 499 | 43.595 | 0.939 | 2.334 | 15.248 | 490.137 | 665.615 |
| WVEN | | | | | | | |
| PVNGAVHLYAQAQTG | 46 | 6.311 | 0.136 | 0.000 | 0.000 | 506.376 | 687.669 |
| WVEN | | | | | | | |
| QLNGAVHLYAQAQTG | 603 | 32.086 | 0.691 | 0.000 | 0.000 | 290.854 | 394.985 |
| WVEN | | | | | | | |
| SLNGAVHLYAQAQTG | 47 | 8.707 | 0.188 | 2.111 | 13.795 | 302.059 | 410.202 |
| WVEN | | | | | | | |
| TLNGAVHLYAQAQTG | 48 | 0.707 | 0.015 | 0.624 | 4.074 | 254.330 | 345.385 |
| WVEN | | | | | | | |
| ALNGAVHLYAQAQTG | 819 | 20.878 | 0.450 | 0.667 | 4.356 | 232.682 | 315.986 |
| WVHN | | | | | | | |
| PHNGAVHLYAQAQTG | 49 | 3.604 | 0.078 | 4.410 | 28.816 | 177.855 | 241.530 |
| WVHN | | | | | | | |
| PINGAVHLYAQAQTG | 50 | 6.387 | 0.138 | 1.083 | 7.076 | 124.477 | 169.043 |
| WVHN | | | | | | | |
| PLKGAVHLYAQAQTG | 51 | 0.986 | 0.021 | 0.516 | 3.368 | 118.166 | 160.472 |
| WVHN | | | | | | | |
| PLNGAAHLYAQAQTG | 52 | 0.757 | 0.016 | 1.024 | 6.693 | 91.230 | 123.892 |
| WVHN | | | | | | | |
| PLNGADHLYAQAQTG | 53 | 0.842 | 0.018 | 0.540 | 3.529 | 15.514 | 21.069 |
| WVHN | | | | | | | |
| PLNGAGHLYAQAQTG | 54 | 1.198 | 0.026 | 1.451 | 9.479 | 106.338 | 144.409 |
| WVHN | | | | | | | |
| PLNGALHLYAQAQTG | 55 | 2.023 | 0.044 | 0.000 | 0.000 | 148.895 | 202.202 |
| WVHN | | | | | | | |
| PLNGAVDLYAQAQTG | 56 | 3.104 | 0.067 | 0.000 | 0.000 | 149.296 | 202.747 |
| WVHN | | | | | | | |
| PLNGAVHHYAQAQTG | 57 | 2.959 | 0.064 | 1.184 | 7.735 | 162.209 | 220.283 |
| WVHN | | | | | | | |
| PLNGAVHIYAQAQTG | 58 | 2.284 | 0.049 | 0.000 | 0.000 | 160.204 | 217.560 |
| WVHN | | | | | | | |
| PLNGAVHLDAQAQTG | 59 | 0.667 | 0.014 | 0.333 | 2.175 | 298.308 | 405.108 |
| WVHN | | | | | | | |
| PLNGAVHLNAQAQTG | 60 | 2.297 | 0.050 | 2.821 | 18.432 | 417.906 | 567.525 |
| WVHN | | | | | | | |
| PLNGAVHLSAQAQTG | 61 | 1.946 | 0.042 | 1.987 | 12.982 | 427.101 | 580.011 |
| WVHN | | | | | | | |
| PLNGAVHLYAKAQTG | 62 | 7.514 | 0.162 | 0.000 | 0.000 | 75.629 | 102.705 |
| WVHN | | | | | | | |
| PLNGAVHLYAQAKTG | 63 | 2.441 | 0.053 | 0.998 | 6.522 | 78.923 | 107.179 |
| WVHN | | | | | | | |
| PLNGAVHLYAQAQAG | 64 | 1.527 | 0.033 | 0.000 | 0.000 | 132.644 | 180.133 |
| WVHN | | | | | | | |
| PLNGAVHLYAQAQRG | 65 | 4.707 | 0.101 | 0.000 | 0.000 | 24.765 | 33.632 |
| WVHN | | | | | | | |
| PLNGAVHLYAQAQSG | 66 | 3.622 | 0.078 | 0.000 | 0.000 | 117.858 | 160.053 |
| WVHN | | | | | | | |
| PLNGAVHLYAQAQTA | 946 | 16.279 | 0.351 | 0.452 | 2.953 | 234.444 | 318.380 |
| WVHN | | | | | | | |
| PLNGAVHLYAQAQTG | 67 | 4.347 | 0.094 | 0.356 | 2.328 | 255.533 | 347.018 |
| CVHN | | | | | | | |
| PLNGAVHLYAQAQTG | 491 | 44.261 | 0.954 | 1.261 | 8.238 | 175.856 | 238.815 |
| GVHN | | | | | | | |
| PLNGAVHLYAQAQTG | 68 | 6.977 | 0.150 | 0.000 | 0.000 | 140.966 | 191.435 |
| LVHN | | | | | | | |
| PLNGAVHLYAQAQTG | 576 | 34.946 | 0.753 | 2.147 | 14.025 | 187.304 | 254.363 |
| SVHN | | | | | | | |
| PLNGAVHLYAQAQTG | 755 | 23.437 | 0.505 | 0.865 | 5.655 | 192.126 | 260.911 |
| WVHN | | | | | | | |
| PLNGAVHLYAQAQVS | 69 | 8.212 | 0.177 | 0.000 | 0.000 | 319.448 | 433.817 |
| AVHN | | | | | | | |
| PLNGAVHLYAQEQTG | 70 | 2.986 | 0.064 | 0.000 | 0.000 | 93.114 | 126.451 |
| WVHN | | | | | | | |
| PLNGAVHLYAQGQTG | 71 | 1.725 | 0.037 | 0.000 | 0.000 | 26.926 | 36.566 |
| WVHN | | | | | | | |
| PLNGAVHLYDQAQTG | 72 | 1.514 | 0.033 | 0.279 | 1.822 | 142.794 | 193.917 |
| WVHN | | | | | | | |
| PLNGAVHLYGQAQTG | 73 | 1.401 | 0.030 | 0.000 | 0.000 | 116.707 | 158.491 |
| WVHN | | | | | | | |
| PLNGAVHLYSQAQTG | 74 | 9.806 | 0.211 | 0.000 | 0.000 | 100.716 | 136.775 |
| WVHN | | | | | | | |
| PLNGAVHRYAQAQTG | 75 | 5.991 | 0.129 | 0.000 | 0.000 | 13.869 | 18.834 |
| WVHN | | | | | | | |
| PLNGAVHVYAQAQTG | 76 | 2.959 | 0.064 | 0.000 | 0.000 | 159.742 | 216.933 |
| WVHN | | | | | | | |
| PLNGAVNLYAQAQTG | 77 | 2.387 | 0.051 | 0.631 | 4.121 | 78.989 | 107.268 |
| WVHN | | | | | | | |
| PLNGAVPLYAQAQTG | 78 | 2.946 | 0.063 | 0.774 | 5.060 | 124.967 | 169.708 |
| WVHN | | | | | | | |
| PLNGAVQLYAQAQTG | 79 | 2.338 | 0.050 | 1.085 | 7.091 | 105.426 | 143.170 |
| WVHN | | | | | | | |
| PLNGGVHLYAQAQTG | 979 | 15.473 | 0.333 | 0.626 | 4.090 | 185.552 | 251.984 |
| WVHN | | | | | | | |
| PLNGSVHLYAQAQTG | 703 | 25.824 | 0.556 | 1.283 | 8.381 | 360.426 | 489.465 |
| WVHN | | | | | | | |
| PLNGTVHLYAQAQTG | 80 | 10.315 | 0.222 | 0.000 | 0.000 | 159.224 | 216.229 |
| WVHN | | | | | | | |
| PRNGAVHLYAQAQTG | 81 | 2.842 | 0.061 | 1.016 | 6.636 | 163.614 | 222.192 |
| WVHN | | | | | | | |
| PVNGAVHLYAQAQTG | 82 | 8.023 | 0.173 | 0.761 | 4.971 | 402.937 | 547.197 |
| WVHN | | | | | | | |
| QLNGAVHLYAQAQTG | 816 | 21.018 | 0.453 | 0.794 | 5.185 | 344.539 | 467.890 |
| WVHN | | | | | | | |
| SLNGAVHLYAQAQTG | 83 | 8.378 | 0.181 | 1.286 | 8.399 | 211.470 | 287.181 |
| WVHN | | | | | | | |
| TLNGAVHLYAQAQTG | 84 | 1.270 | 0.027 | 0.252 | 1.648 | 136.758 | 185.720 |
| WVHN | | | | | | | |
| ALNGAVHLYAQAQTG | 1081 | 13.477 | 0.290 | 0.084 | 0.546 | 9.629 | 13.077 |
| WVKN | | | | | | | |
| ALNGAVNLYAQAQTG | 85 | 4.203 | 0.091 | 0.000 | 0.000 | 3.919 | 5.323 |
| WVKN | | | | | | | |
| KLNGAVHLYAQAQTG | 86 | 7.153 | 0.154 | 0.000 | 0.000 | 9.576 | 13.004 |
| WVKN | | | | | | | |
| PHNGAVHLYAQAQTG | 87 | 1.248 | 0.027 | 0.378 | 2.467 | 5.661 | 7.688 |
| WVKN | | | | | | | |
| PINGAVHLYAQAQTG | 88 | 7.644 | 0.165 | 0.143 | 0.933 | 7.560 | 10.267 |
| WVKN | | | | | | | |
| PLKGAVHLYAQAQTG | 89 | 0.748 | 0.016 | 0.103 | 0.675 | 7.088 | 9.626 |
| WVKN | | | | | | | |
| PLNAAVHLYAQAQTG | 90 | 2.473 | 0.053 | 0.000 | 0.000 | 18.662 | 25.343 |
| WVKN | | | | | | | |
| PLNGAAHLYAQAQTG | 91 | 0.901 | 0.019 | 0.390 | 2.547 | 5.894 | 8.004 |
| WVKN | | | | | | | |
| PLNGADHLYAQAQTG | 92 | 3.270 | 0.070 | 0.316 | 2.064 | 0.970 | 1.317 |
| WVKN | | | | | | | |
| PLNGAGHLYAQAQTG | 93 | 0.378 | 0.008 | 0.209 | 1.367 | 11.306 | 15.354 |
| WVKN | | | | | | | |
| PLNGALHLYAQAQTG | 859 | 19.306 | 0.416 | 0.059 | 0.387 | 19.870 | 26.984 |
| WVKN | | | | | | | |
| PLNGAVDLYAQAQTG | 94 | 0.937 | 0.020 | 0.000 | 0.000 | 5.016 | 6.812 |
| WVKN | | | | | | | |
| PLNGAVHHYAQAQTG | 95 | 1.572 | 0.034 | 0.122 | 0.800 | 8.227 | 11.173 |
| WVKN | | | | | | | |
| PLNGAVHIYAQAQTG | 96 | 11.752 | 0.253 | 0.310 | 2.024 | 10.015 | 13.600 |
| WVKN | | | | | | | |
| PLNGAVHLDAQAQTG | 97 | 0.455 | 0.010 | 0.132 | 0.864 | 14.604 | 19.832 |
| WVKN | | | | | | | |
| PLNGAVHLNAQAQTG | 98 | 1.725 | 0.037 | 0.739 | 4.830 | 21.040 | 28.572 |
| WVKN | | | | | | | |
| PLNGAVHLSAQAQTG | 99 | 0.653 | 0.014 | 0.523 | 3.415 | 16.268 | 22.092 |
| WVKN | | | | | | | |
| PLNGAVHLYAEAQTG | 100 | 0.752 | 0.016 | 0.000 | 0.000 | 0.825 | 1.120 |
| WVKN | | | | | | | |
| PLNGAVHLYAHAQTG | 101 | 8.658 | 0.187 | 0.345 | 2.253 | 1.734 | 2.355 |
| WVKN | | | | | | | |
| PLNGAVHLYAKAQTG | 649 | 28.559 | 0.615 | 0.278 | 1.817 | 1.532 | 2.081 |
| WVKN | | | | | | | |
| PLNGAVHLYALAQTG | 102 | 2.311 | 0.050 | 0.000 | 0.000 | 9.892 | 13.433 |
| WVKN | | | | | | | |
| PLNGAVHLYAPAQTG | 103 | 3.766 | 0.081 | 0.000 | 0.000 | 5.697 | 7.737 |
| WVKN | | | | | | | |
| PLNGAVHLYAQAETG | 104 | 0.360 | 0.008 | 0.213 | 1.394 | 2.738 | 3.718 |
| WVKN | | | | | | | |
| PLNGAVHLYAQAQKG | 105 | 7.865 | 0.169 | 0.099 | 0.648 | 1.486 | 2.018 |
| WVKN | | | | | | | |
| PLNGAVHLYAQAQLS | 566 | 36.293 | 0.782 | 0.000 | 0.000 | 134.753 | 182.997 |
| PVKN | | | | | | | |
| PLNGAVHLYAQAQLS | 960 | 15.955 | 0.344 | 0.000 | 0.000 | 51.408 | 69.813 |
| SVKN | | | | | | | |
| PLNGAVHLYAQAQPG | 106 | 10.171 | 0.219 | 0.000 | 0.000 | 13.928 | 18.914 |
| WVKN | | | | | | | |
| PLNGAVHLYAQAQSG | 107 | 3.450 | 0.074 | 0.000 | 0.000 | 7.922 | 10.758 |
| WVKN | | | | | | | |
| PLNGAVHLYAQAQSS | 108 | 11.748 | 0.253 | 0.000 | 0.000 | 8.691 | 11.802 |
| LVKN | | | | | | | |
| PLNGAVHLYAQAQSS | 109 | 5.856 | 0.126 | 0.169 | 1.107 | 10.452 | 14.194 |
| SVKN | | | | | | | |
| PLNGAVHLYAQAQTA | 110 | 4.473 | 0.096 | 0.000 | 0.000 | 14.173 | 19.247 |
| CVKN | | | | | | | |
| PLNGAVHLYAQAQTA | 763 | 23.248 | 0.501 | 0.000 | 0.000 | 11.433 | 15.526 |
| SVKN | | | | | | | |
| PLNGAVHLYAQAQTA | 828 | 20.599 | 0.444 | 0.333 | 2.174 | 6.667 | 9.054 |
| WVKN | | | | | | | |
| PLNGAVHLYAQAQTE | 111 | 0.721 | 0.016 | 0.000 | 0.000 | 14.566 | 19.781 |
| WVKN | | | | | | | |
| PLNGAVHLYAQAQTG | 112 | 8.239 | 0.178 | 0.000 | 0.000 | 10.794 | 14.659 |
| AVKN | | | | | | | |
| PLNGAVHLYAQAQTG | 113 | 8.144 | 0.175 | 0.222 | 1.452 | 14.865 | 20.187 |
| CVKN | | | | | | | |
| PLNGAVHLYAQAQTG | 529 | 40.644 | 0.876 | 0.167 | 1.092 | 8.407 | 11.417 |
| GVKN | | | | | | | |
| PLNGAVHLYAQAQTG | 114 | 10.090 | 0.217 | 0.145 | 0.945 | 10.946 | 14.864 |
| LVKN | | | | | | | |
| PLNGAVHLYAQAQTG | 1100 | 13.059 | 0.281 | 0.000 | 0.000 | 2.607 | 3.541 |
| RVKN | | | | | | | |
| PLNGAVHLYAQAQTG | 631 | 29.707 | 0.640 | 0.516 | 3.369 | 7.169 | 9.735 |
| SVKN | | | | | | | |
| PLNGAVHLYAQAQTG | 765 | 23.167 | 0.499 | 0.260 | 1.700 | 10.937 | 14.852 |
| WVKN | | | | | | | |
| PLNGAVHLYAQAQTL | 720 | 25.032 | 0.539 | 1.771 | 11.569 | 10.981 | 14.913 |
| SVKN | | | | | | | |
| PLNGAVHLYAQAQTR | 1033 | 14.369 | 0.310 | 1.308 | 8.546 | 1.210 | 1.643 |
| WVKN | | | | | | | |
| PLNGAVHLYAQAQTS | 700 | 26.099 | 0.562 | 0.000 | 0.000 | 29.844 | 40.528 |
| SVKN | | | | | | | |
| PLNGAVHLYAQAQTS | 1037 | 14.347 | 0.309 | 0.000 | 0.000 | 12.484 | 16.953 |
| TVKN | | | | | | | |
| PLNGAVHLYAQAQTS | 115 | 4.212 | 0.091 | 0.000 | 0.000 | 34.651 | 47.056 |
| WVKN | | | | | | | |
| PLNGAVHLYAQAQTV | 625 | 30.131 | 0.649 | 0.000 | 0.000 | 22.666 | 30.781 |
| AVKN | | | | | | | |
| PLNGAVHLYAQAQTV | 325 | 69.266 | 1.492 | 0.231 | 1.508 | 22.335 | 30.332 |
| SVKN | | | | | | | |
| PLNGAVHLYAQAQTV | 771 | 22.784 | 0.491 | 0.000 | 0.000 | 1.612 | 2.189 |
| WVKN | | | | | | | |
| PLNGAVHLYAQAQVS | 610 | 31.581 | 0.680 | 0.354 | 2.310 | 21.595 | 29.326 |
| AVKN | | | | | | | |
| PLNGAVHLYAQAQVS | 558 | 37.158 | 0.801 | 0.000 | 0.000 | 40.525 | 55.033 |
| PVKN | | | | | | | |
| PLNGAVHLYAQEQTG | 116 | 7.694 | 0.166 | 0.000 | 0.000 | 1.548 | 2.102 |
| WVKN | | | | | | | |
| PLNGAVHLYAQSQTG | 117 | 7.014 | 0.151 | 0.000 | 0.000 | 5.931 | 8.054 |
| WVKN | | | | | | | |
| PLNGAVHLYDQAQTG | 118 | 2.761 | 0.059 | 0.146 | 0.956 | 7.745 | 10.518 |
| WVKN | | | | | | | |
| PLNGAVHLYEQAQTG | 119 | 7.658 | 0.165 | 0.000 | 0.000 | 7.124 | 9.675 |
| WVKN | | | | | | | |
| PLNGAVHLYGQAQTG | 120 | 0.874 | 0.019 | 0.000 | 0.000 | 5.745 | 7.802 |
| WVKN | | | | | | | |
| PLNGAVHLYSQAQTG | 121 | 6.910 | 0.149 | 0.000 | 0.000 | 11.682 | 15.865 |
| LVKN | | | | | | | |
| PLNGAVHLYSQAQTG | 122 | 7.536 | 0.162 | 0.245 | 1.599 | 4.415 | 5.996 |
| WVKN | | | | | | | |
| PLNGAVHLYTQAQTG | 123 | 0.595 | 0.013 | 0.166 | 1.086 | 10.157 | 13.793 |
| WVKN | | | | | | | |
| PLNGAVHPYAQAQTG | 124 | 0.788 | 0.017 | 0.178 | 1.162 | 3.527 | 4.790 |
| WVKN | | | | | | | |
| PLNGAVHRSLQAQTG | 125 | 0.153 | 0.003 | 0.449 | 2.931 | 7.938 | 10.780 |
| WVKN | | | | | | | |
| PLNGAVHRYAQAQTG | 126 | 1.878 | 0.040 | 0.000 | 0.000 | 2.446 | 3.322 |
| WVKN | | | | | | | |
| PLNGAVHVYAQAQTG | 127 | 1.937 | 0.042 | 0.123 | 0.802 | 7.871 | 10.689 |
| WVKN | | | | | | | |
| PLNGAVLLYAQAQTG | 128 | 4.149 | 0.089 | 0.211 | 1.379 | 3.878 | 5.266 |
| WVKN | | | | | | | |
| PLNGAVNLYAQAQTG | 129 | 3.505 | 0.076 | 0.000 | 0.000 | 1.145 | 1.555 |
| CVKN | | | | | | | |
| PLNGAVNLYAQAQTG | 130 | 4.586 | 0.099 | 0.262 | 1.710 | 2.124 | 2.884 |
| WVKN | | | | | | | |
| PLNGAVNLYDQAQTG | 131 | 2.486 | 0.054 | 0.000 | 0.000 | 9.468 | 12.858 |
| WVKN | | | | | | | |
| PLNGAVPLYAQAQTG | 132 | 1.622 | 0.035 | 0.000 | 0.000 | 7.002 | 9.509 |
| WVKN | | | | | | | |
| PLNGAVQLYAQAQTG | 133 | 2.014 | 0.043 | 0.381 | 2.487 | 11.888 | 16.145 |
| WVKN | | | | | | | |
| PLNGAVRSTAQAQTG | 134 | 0.505 | 0.011 | 0.662 | 4.326 | 24.950 | 33.883 |
| WVKN | | | | | | | |
| PLNGAVSLRAQAQTG | 135 | 0.725 | 0.016 | 1.267 | 8.277 | 6.291 | 8.543 |
| WVKN | | | | | | | |
| PLNGAVSSRAQAQTG | 136 | 0.739 | 0.016 | 1.091 | 7.130 | 19.478 | 26.452 |
| WVKN | | | | | | | |
| PLNGDVHLYAQAQTG | 14 | 2.905 | 0.063 | 0.000 | 0.000 | 10.919 | 14.829 |
| CVKN | | | | | | | |
| PLNGDVHLYAQAQTG | 15 | 2.559 | 0.055 | 0.235 | 1.539 | 9.737 | 13.224 |
| WVKN | | | | | | | |
| PLNGGVHLYAQAQTG | 864 | 19.077 | 0.411 | 0.292 | 1.905 | 4.576 | 6.214 |
| WVKN | | | | | | | |
| PLNGPVHLYAQAQTG | 16 | 2.216 | 0.048 | 0.000 | 0.000 | 5.169 | 7.019 |
| WVKN | | | | | | | |
| PLNGSVHLYAQAQTG | 17 | 5.707 | 0.123 | 0.000 | 0.000 | 21.281 | 28.900 |
| CVKN | | | | | | | |
| PLNGSVHLYAQAQTG | 791 | 22.050 | 0.475 | 0.184 | 1.202 | 13.110 | 17.803 |
| WVKN | | | | | | | |
| PLNGTVHLYAQAQTG | 978 | 15.482 | 0.334 | 0.000 | 0.000 | 17.576 | 23.868 |
| WVKN | | | | | | | |
| PLNSAVHLYAQAQTG | 1174 | 1.468 | 0.032 | 0.000 | 0.000 | 3.674 | 4.989 |
| WVKN | | | | | | | |
| PLSGAVHLYAQAQTG | 1175 | 0.680 | 0.015 | 0.000 | 0.000 | 8.111 | 11.015 |
| WVKN | | | | | | | |
| PPNGAVHLYAQAQTG | 1176 | 0.829 | 0.018 | 0.546 | 3.567 | 9.008 | 12.233 |
| WVKN | | | | | | | |
| PRNGAVHLYAQAQTG | 1177 | 1.194 | 0.026 | 0.421 | 2.751 | 9.143 | 12.417 |
| WVKN | | | | | | | |
| PVNGAVHLYAQAQTG | 1178 | 8.167 | 0.176 | 0.145 | 0.946 | 11.979 | 16.268 |
| WVKN | | | | | | | |
| QLNGAVHIYAQAQTG | 1179 | 6.982 | 0.150 | 0.000 | 0.000 | 14.405 | 19.563 |
| WVKN | | | | | | | |
| QLNGAVHLYAQAQTG | 1180 | 2.446 | 0.053 | 0.000 | 0.000 | 14.453 | 19.627 |
| CVKN | | | | | | | |
| QLNGAVHLYAQAQTG | 1181 | 7.518 | 0.162 | 0.000 | 0.000 | 1.613 | 2.190 |
| LVKN | | | | | | | |
| QLNGAVHLYAQAQTG | 966 | 15.815 | 0.341 | 1.130 | 7.380 | 3.272 | 4.443 |
| SVKN | | | | | | | |
| QLNGAVHLYAQAQTG | 1032 | 14.378 | 0.310 | 0.137 | 0.893 | 10.495 | 14.253 |
| WVKN | | | | | | | |
| QLNGAVHLYDQAQTG | 1182 | 2.378 | 0.051 | 0.000 | 0.000 | 1.224 | 1.663 |
| WVKN | | | | | | | |
| RLNGAVHLYAQAQTG | 1183 | 3.131 | 0.067 | 0.000 | 0.000 | 4.894 | 6.646 |
| WVKN | | | | | | | |
| SINGAVHLYAQAQTG | 1184 | 1.234 | 0.027 | 0.409 | 2.671 | 4.660 | 6.328 |
| WVKN | | | | | | | |
| SLNGAVHLYAQAQTG | 1185 | 6.104 | 0.132 | 0.328 | 2.143 | 11.122 | 15.104 |
| WVKN | | | | | | | |
| SLNGAVNLYAQAQTG | 1186 | 1.387 | 0.030 | 0.833 | 5.444 | 5.215 | 7.083 |
| WVKN | | | | | | | |
| TLNGAVHLYAQAQTG | 1187 | 2.176 | 0.047 | 0.108 | 0.707 | 5.355 | 7.272 |
| WVKN | | | | | | | |
| TLNGAVNLYAQAQTG | 1188 | 1.333 | 0.029 | 0.000 | 0.000 | 1.292 | 1.754 |
| WVKN | | | | | | | |
| PLNGAVHHYAQAQTG | 1189 | 0.937 | 0.020 | 0.000 | 0.000 | 982.466 | 1334.208 |
| WVPN | | | | | | | |
| PLNGAVHLNAQAQTG | 1190 | 3.698 | 0.080 | 3.072 | 20.072 | 5448.821 | 7399.606 |
| WVPN | | | | | | | |
| PLNGAVHLSAQAQTG | 1191 | 3.356 | 0.072 | 3.362 | 21.966 | 5527.158 | 7505.989 |
| WVPN | | | | | | | |
| PLNGAVHLYAQAQTG | 1192 | 8.959 | 0.193 | 0.969 | 6.329 | 3019.591 | 4100.664 |
| CVPN | | | | | | | |
| PLNGAVHLYAQAQTG | 314 | 71.770 | 1.546 | 2.071 | 13.534 | 2408.300 | 3270.518 |
| WVPN | | | | | | | |
| PLNGAVNLYAQAQTG | 1193 | 5.356 | 0.115 | 1.809 | 11.822 | 2103.432 | 2856.501 |
| WVPN | | | | | | | |
| PLNGTVHLYAQAQTG | 885 | 18.270 | 0.394 | 0.000 | 0.000 | 4688.758 | 6367.425 |
| WVPN | | | | | | | |
| PVNGAVHLYAQAQTG | 1016 | 14.568 | 0.314 | 0.000 | 0.000 | 1269.496 | 1724.000 |
| WVPN | | | | | | | |
| QLNGAVHLYAQAQTG | 648 | 28.572 | 0.616 | 0.000 | 0.000 | 14152.524 | 19219.405 |
| WVPN | | | | | | | |
| SLNGAVHLYAQAQTG | 1058 | 13.914 | 0.300 | 1.876 | 12.256 | 1800.081 | 2444.545 |
| WVPN | | | | | | | |
| PLNGAVHLYAQAQTG | 476 | 46.410 | 1 | 0.153 | 1 | 0.736 | 1 |
| WVQN | | | | | | | |
| AQAQAQTGWVQN | 1194 | 1 | 0.0215 | 1 | 6.533 | 1 | 1.358 |

### Example 5. Dose-Response Evaluation of TTD-001 in Non-Human Primates (NHPs)

This Example investigates the minimal dose of an AAV particle comprising a TTD-001 capsid variant (SEQ ID NO: 3623 (DNA) and 3636 (amino acid), comprising SEQ ID NO: 3648)) that is sufficient to achieve near-physiological expression of a payload, e.g., a single stranded payload, in the central nervous system of adult cynomolgus macaques (Macaca fascicularis) via intravenous systemic delivery.

AAV particles comprising the TTD-001 capsid variant comprising a single stranded viral genome encoding a hemagglutinin (HA)-tagged NHP protein under the control of a ubiquitous CBA promoter were injected intravenously into adult male NHPs (cynomolgus macaque) (n=3, 5-7 years of age) at various doses spanning a 30-fold range, which included 6.7e11 VG/kg, 2e12 VG/kg, 6.7e12 VG/kg, and 2e13 VG·kg. The in-life period was 28 days and then various CNS and peripheral tissues were collected for measuring transgene mRNA expression by RT-qPCR, viral DNA levels by ddPCR, transgene protein expression by ELISA, and biodistribution by immunohistochemistry (staining with an anti-HA antibody).

Widespread transgene expression was detected in the spinal cord and the brain the NHPs at doses of 2e12 VG/kg and above, especially in the putamen, thalamus, globus pallidus and brainstem (Tables 25-27). Viral DNA and mRNA were readily detectable in all NHPs and showed a consistent dose response (Table 25, Table 26).

More specifically, in the brain, dose-dependent distribution of the AAV particles comprising the TTD-001 capsid was observed in the cortical regions (frontal, motor, and somatosensory), caudate, putamen, thalamus, substantia nigra, globus pallidus, hippocampus, amygdala, hypothalamus, cerebellar cortex, and dentate nucleus. Additionally, for each dose administered, there was comparable distribution of the AAV particles comprising the TTD-001 capsid in each brain region, including the cortex as well as the deeper brain regions such as the caudate, putamen, thalamus, substantia nigra, globus pallidus, hippocampus, amygdala, hypothalamus, and dentate nucleus (Table 25).

With respect to the spinal cord, dose-dependent distribution of the AAV particles comprising the TTD-001 capsid was observed in the cervical, thoracic, and lumbar spinal cord regions and the relative distribution across all these regions was similar for each dosing group. As shown in Table 25, low biodistribution was measured in the DRG, but a dose-dependent distribution of the AAV particles comprising the TTD-001 capsid was observed in the cervical, thoracic, and lumbar DRG regions and the relative distribution across all DRG regions was similar for each dosing group.

With respect to the peripheral tissues, a dose-dependent distribution of the AAV particles comprising the TTD-001 capsid was observed in the liver, hear, and the vastus lateralis (muscle) (Table 25).

TABLE 25
Quantification of viral genomes (biodistribution) by
ddPCR following intravenous administration of various
doses of AAV particles comprising a TTD-001 capsid

| | Quantification of viral genomes in the Brain | | | | | | |
|---|---|---|---|---|---|---|---|
| | (VG/diploid cell) | | | | | | |
| | | | Somato- | | | | |
| Dose | Frontal | Motor | sensory | | Puta- | Thala- | Dentate |
| (VG/kg) | Cortex | Cortex | Cortex | Caudate | men | mus | Nucleus |
| 6.7e11 | 0.01 | 0.11 | 0.07 | 0.16 | 0.13 | 0.17 | 0.03 |
| 2e12 | 2.0 | 1.72 | 0.92 | 1.27 | 0.89 | 1.16 | 0.42 |
| 6.7e12 | 4.5 | 4.20 | 3.44 | 3.44 | 2.74 | 3.04 | 2.27 |
| 2e13 | 6.7 | 7.50 | 3.04 | 6.32 | 4.94 | 6.34 | 4.14 |

| Dose | Substantia | Globus | Hippo- | Amyg- | Hypo- | Cerebellar |
|---|---|---|---|---|---|---|
| (VG/kg) | Nigra | Pallidus | campus | dala | thalamus | Cortex |
| 6.7e11 | 0.12 | 0.10 | 0.1 | 0.16 | 0.14 | 0.02 |
| 2e12 | 1.12 | 1.28 | 2.1 | 2.83 | 1.77 | 0.13 |
| 6.7e12 | 2.33 | 2.47 | 2.6 | 4.38 | 2.37 | 0.53 |
| 2e13 | 5.81 | 4.43 | 5.5 | 6.13 | 4.63 | 4.18 |

| | Quantification of viral genomes in the | | | | | |
|---|---|---|---|---|---|---|
| | Spinal Cord and DRG (VG/diploid cell) | | | | | |
| | Cervical | Thoracic | Lumbar | | | |
| Dose | Spinal | Spinal | Spinal | Cervical | Thoracic | Lumbar |
| (VG/kg) | Cord | Cord | Cord | DRG | DRG | DRG |
| 6.7e11 | 0.06 | 0.03 | 0.05 | 0.002 | 0.003 | 0.007 |
| 2e12 | 0.74 | 0.42 | 0.64 | 0.007 | 0.006 | 0.019 |
| 6.7e12 | 1.35 | 0.94 | 1.58 | 0.015 | 0.022 | 0.033 |
| 2e13 | 5.35 | 3.48 | 6.48 | 0.060 | 0.079 | 0.070 |

| | |
|---|---|
| | Quantification of viral genomes |
| Dose | in the peripheral tissues (VG/diploid cell) |

| (VG/kg) | Heart | Liver | Kidney | Vastus lateralis (muscle) |
|---|---|---|---|---|
| 6.7e11 | 0.11 | 1.2 | 0.04 | 0.002 |
| 2e12 | 0.10 | 12.4 | 0.71 | 0.02 |
| 6.7e12 | 0.30 | 68.5 | 1.18 | 0.06 |
| 2e13 | 0.56 | 132.3 | 0.84 | 0.15 |

Additionally, dose-dependent transgene mRNA expression by the AAV particles comprising the TTD-001 capsid was observed in the brain, spinal cord, DRG, and peripheral tissues (Table 44). The lowest dose of the AAV particles comprising the TTD-001 capsid protein resulted in higher transgene mRNA and protein expression than a 30-fold higher dose of wild-type AAV9. Comparison of the transgene mRNA with the matching endogenous transcript indicated that a dose of 2e12VG/kg was sufficient to achieve supra-physiological levels in the central nervous system (CNS), while showing low transduction in the liver and the dorsal root ganglia (DRG) (Table 26).

TABLE 26
Quantification of transgene mRNA by RT-qPCR following
intravenous administration of various doses of
AAV particles comprising a TTD-001 capsid
Transgene mRNA relative to housekeeping gene

| | Dose (VG/kg) | | | |
|---|---|---|---|---|
| | 6.7e11 | 2e12 | 6.7e12 | 2e13 |
| Tissue | VG/kg | VG/kg | VG/kg | VG/kg |
| Frontal Cortex | 0.02 | 0.29 | 3.42 | 4.51 |
| Motor Cortex | 0.2 | 1.65 | 9.42 | 26.5 |
| Putamen | 0.11 | 0.38 | 1.76 | 2.52 |
| Dentate Nucleus | 0.01 | 0.26 | 4.38 | 15.8 |
| Cervical Spinal Cord | 0.07 | 0.63 | 2.25 | 8.26 |
| Thoracic Spinal Cord | 0.06 | 0.72 | 2.00 | 5.37 |
| Lumbar Spinal Cord | 0.10 | 0.83 | 6.71 | 27.22 |
| Cervical DRG | 0.02 | 0.29 | 0.99 | 3.58 |
| Thoracic DRG | 0.01 | 0.28 | 1.08 | 4.36 |
| Lumbar DRG | 0.02 | 0.56 | 2.21 | 4.44 |
| Heart | 0.002 | 0.33 | 5.37 | 11.45 |
| Liver | 0.04 | 0.3 | 1.43 | 2.22 |

Transgene mRNA vs. endogenous transcript
(fold change relative to vehicle control)

| | Dose (VG/kg) | | | | |
|---|---|---|---|---|---|
| | | 6.7e11 | 2e12 | 6.7e12 | 2e13 |
| Tissue | Vehicle | VG/kg | VG/kg | VG/kg | VG/kg |
| Frontal Cortex | 1.0 | 0.97 | 2.04 | 12.79 | 17.04 |
| Motor Cortex | 1.0 | 2.3 | 9.4 | 42.0 | 118.3 |
| Putamen | 1.0 | 1.59 | 3.00 | 11.25 | 16.57 |
| Dentate Nucleus | 1.0 | 0.7 | 1.7 | 17.0 | 59.9 |
| Cervical Spinal Cord | 1.0 | 1.18 | 2.65 | 12.57 | 43.16 |
| Thoracic Spinal Cord | 1.0 | 1.07 | 2.26 | 10.61 | 27.96 |
| Lumbar Spinal Cord | 1.0 | 1.16 | 3.06 | 31.30 | 122.75 |
| Cervical DRG | 1.0 | 1.0 | 1.63 | 3.24 | 9.09 |
| Thoracic DRG | 1.0 | 0.98 | 1.4 | 3.02 | 7.72 |
| Lumbar DRG | 1.0 | 1.05 | 2.3 | 4.78 | 11.72 |
| Heart | 1.0 | 1.34 | 1.67 | 6.55 | 13.57 |
| Liver | 1.0 | 0.9 | 1.2 | 2.8 | 3.2 |

TABLE 27
Quantification of total transgene protein expression in the peripheral
tissues by ELISA following intravenous administration of various
doses of AAV particles comprising a TTD-001 capsid

| | Cervical DRG | Heart | Liver |
|---|---|---|---|
| | (Transgene | (Transgene | (Transgene |
| Dose | ng/mL relative | ng/mL relative | ng/mL relative |
| (VG/kg) | to vehicle) | to vehicle) | to vehicle) |
| Vehicle | 1.0 | 1.0 | 1.0 |
| 6.7e11 | 1.1 | 1.0 | 0.87 |
| 2e12 | 1.5 | 1.04 | 1.00 |
| 6.7e12 | 1.7 | 2.23 | 0.93 |
| 2e13 | 7.4 | 3.69 | 1.24 |

By immunohistochemistry (JHC), widespread transduction by AAV particles comprising the TTD-001 capsid variant was observed in multiple brain regions of the NHPs as compared to AAV9 at all doses administered, particularly at the medium to high doses (2e12 VG/kg, 6.7e12 VG/kg, and 2e13 VG/kg). By JHC, dose dependent expression of AAV particles comprising the TTD-001 capsid variant was observed in the brain, specifically in the temporal cortex, caudate, putamen, thalamus, substantia nigra, hippocampus, and cerebellar. Morphologically, transgene expression was observed in the neuronal cell body and the neuropil from neurons in these brain regions, including the Purkinje neurons in the cerebellar cortex and the neurons deep in the cerebellar nuclei. In the brain stem, the transgene expression was observed in various structures including the gracile-nuclei, cuneate-nuclei, and the Inferior Olivary complex.

In the spinal cord of the NHPs, dose dependent transduction was also observed in the cervical, lumbar, and thoracic regions when measured by IHC, with the most intense and widespread staining occurring at the 6.7e12 VG/kg and 2e13 VG/kg doses. Substantial staining of the motor neurons in the spinal cord was also observed at the lower dose of 2e12 VG/kg. Furthermore, the cellular tropism of the TTD-001 capsid in the spinal cord appeared to be largely neuronal and neuropil at all doses in all regions (e.g., cervical, thoracic, and lumbar) investigated.

In the DRG of the NHPs, dose dependent transduction was also observed in the cervical, lumbar, and thoracic regions, with the most staining occurring at the 6.7e12 VG/kg and 2e13 VG/kg doses. The lower dose of 2e12 VG/kg showed significantly less staining and was comparable to particles comprising an AAV9 capsid that were administered at a higher dose of 2e13 VG/kg. The cellular tropism of the TTD-001 capsid in the DRG appeared to be largely neuronal at all doses in all regions investigated.

Transduction of AAV particles comprising the TTD-001 capsid variant was also measured by IHC in various peripheral tissues of the NHPs. In the liver, the transduction observed was more variable but appeared to follow a dose-dependent trend and appeared to be lower than by particles comprising an AAV9 capsid that were administered at a dose of 2e13 VG/kg. Minimal staining was observed in the quadriceps at all doses tested. In the heart, a dose-dependent trend in transduction was also observed.

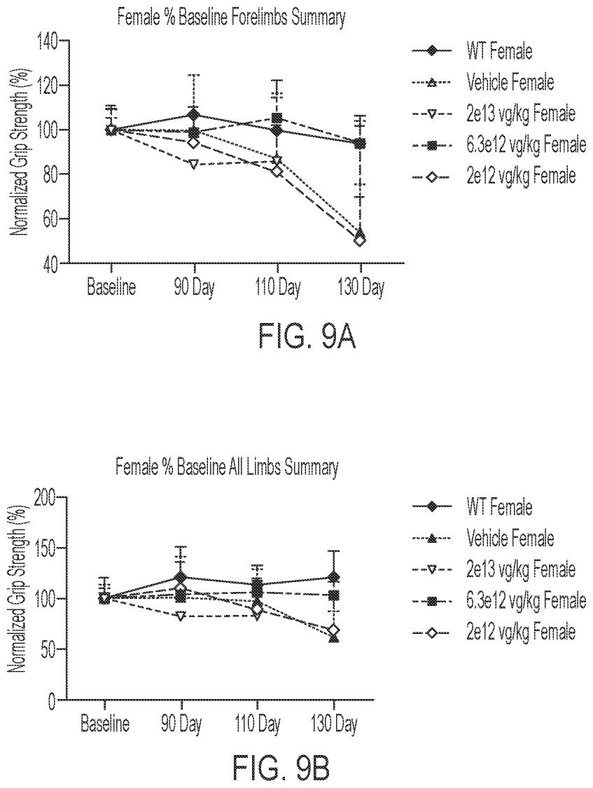

Additionally, the staining of various cells in the brain and/or spinal cord following transduction with the AAV particles comprising the TTD-001 capsid at the doses investigated was quantified. As shown in FIG. 9B, a dose of 2e13 VG/kg was sufficient to transduce >40% of total cells in highly permissive brain regions (thalamus, caudate, putamen) and >20% total cells in less permissive regions (entorhinal cortex, auditory cortex, hippocampus). Even at a lower dose of 6.7e12, this was sufficient to transduce >20% of cells in the thalamus, caudate, putamen, and cerebellum (FIG. 9A). As shown in FIG. 9C, the dose of 2e13 VG/kg also resulted in transduction of >90% SMI311-positive neurons in the thalamus, dentate and spinal cord.

Together, these data demonstrate that variant AAV capsids, including TTD-001, can achieve a large improvement of their therapeutic index by retaining strong efficacy at low dose.

### Example 6. Spatial-omics Study of SOD1 mRNA Knockdown in Lumbar Motor Neurons after Intra-Cisterna Magna Delivery of an AAV in Cynomolgus Macaques

The goal of these experiments was to determine if intra-cisterna magna (ICM) delivery of an AAV.amiRNA vector to non-human primates (NHP; cynomolgus macaques) could achieve knockdown of endogenous SOD1 in lumbar spinal cord motor neurons in a dose-dependent manner. Additionally, several technologies with the potential for high spatial resolution were compared for their ability to detect SOD1 knockdown in motor neurons including: Digital Spatial Profiler (DSP, Nanostring Technologies), in-situ hybridization (ISH; Advanced Cell Diagnostics, RNAscope), Laser Capture Micro-dissection (LCM) followed by RT-qPCR, and spatial transcriptomics (10× Genomics, Visium platform).

Targeted delivery of adeno-associated virus (AAV) gene therapy using an AAV9-SOD1-shRNA vector to the motor cortex of SOD1^{G93A} rats, a model of human SOD1-ALS, resulted in delay of disease onset, expansion of lifespan and enhanced survival of spinal motor neurons (Thomsen et al., “Delayed Disease Onset and Extended Survival in the SOD1^{G93A} Rat Model of Amyotrophic Lateral Sclerosis after Suppression of Mutant SOD1 in the Motor Cortex” J. Neurosci. 34(47):15587-15600 (2014), which is hereby incorporated by reference in its entirety). Intracerebroventricular (ICV) antisense oligonucleotide (ASO) therapy targeting SOD1 reverses loss of muscle action potentials in SOD1^{G93A} mice and attenuates neurodegeneration (McCampbell et al. “Antisense oligonucleotides extend survival and reverse decrement in muscle response in ALS models” J Clin Invest. 128(8):3558-3567 (2018), which is hereby incorporated by reference in its entirety).

It is herein hypothesized that therapeutic strategies reducing mutant SOD1 could provide clinically beneficial outcomes to ALS patients. Patzke reported on the selection of a gene therapy vector targeting SOD1 using an artificial miRNA approach (amiRNA) and showed significant reduction of SOD1 after intra-parenchymal dosing into the pig spinal cord (Patzke et al., “C75 Robust SOD1 knockdown in large mammal spinal cord using a novel delivery paradigm with AAV gene therapy targeting SOD1 for the treatment of SOD1 ALS” Platform Communication, Preclinical therapeutic strategies, 59, MND and ALS Conference (2018), which is hereby incorporated by reference in its entirety).

The present experiment was designed to assess the utility of ICM delivery of an AAV gene therapy vector to lower SOD1 in motor neurons of cynomolgus macaques, as well as to compare the sensitivity and specificity of spatial-omics technologies to study SOD1 knockdown, specifically in motor neurons of the lumbar spinal cord.

Two doses of the AAV.SOD1-amiRNA vector or the vehicle (control) were administered via intra-cisterna magna (ICM) route in NHP and tissues were collected after 28 days for subsequent analysis. 14 um frozen cross sections of the hemi-sected Lumbar L2 spinal cords were used for Laser Capture Microdissection (LCM) to collect approximately 180 motor neurons from each animal for subsequent SOD1 mRNA analysis by RT-qPCR. 5 um formalin fixed paraffin embedded (FFPE) cross sections of the hemi-sected Lumbar L2 spinal cords were used for Digital Spatial Profiler (DSP) to collect approximately 96 motor neurons from each animal for SOD1 gene counts using nCounter (Nanostring). 5 um FFPE cross sections of the hemi-sected Lumbar L2 spinal were used for ISH (RNAscope) to study SOD1 mRNA expression in motor neurons identified by ChAT immunohistochemistry (ChAT IHC). Additionally, the spatial-omics technologies were compared to investigate the potential for high spatial resolution assays to detect SOD1 knockdown in motor neurons.

Digital Spatial Profiling of ChAT-positive lumbar motor neurons showed substantial and dose-dependent knockdown of SOD1 mRNA in fixed sections. Similarly, LCM motor neuron pools demonstrated SOD1 reduction in frozen sections. For both DSP and LCM methods, optimal housekeeping genes for normalization were determined. Furthermore, RNAscope in-situ hybridization showed a decrease in SOD1 mRNA signal in large ventral horn motor neurons of the lumbar spinal cord.

The data showed dose-dependent and robust knockdown of endogenous SOD1 in the motor neurons of Lumbar L2 spinal cord after intra-cisterna magna (ICM) delivery of AAV.SOD1 amiRNA to non-human primates (NHP; cynomolgus macaques).

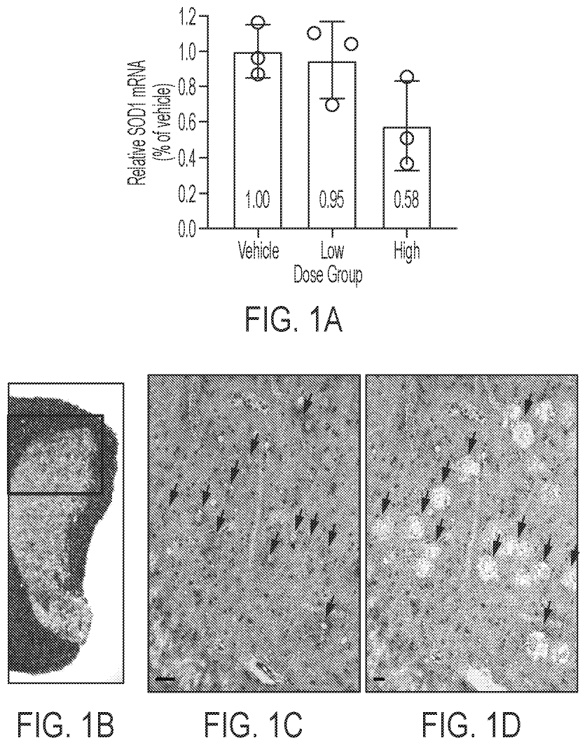

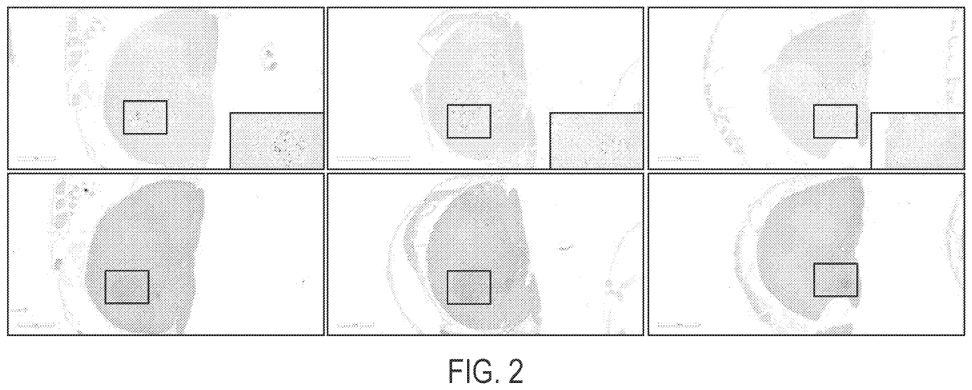

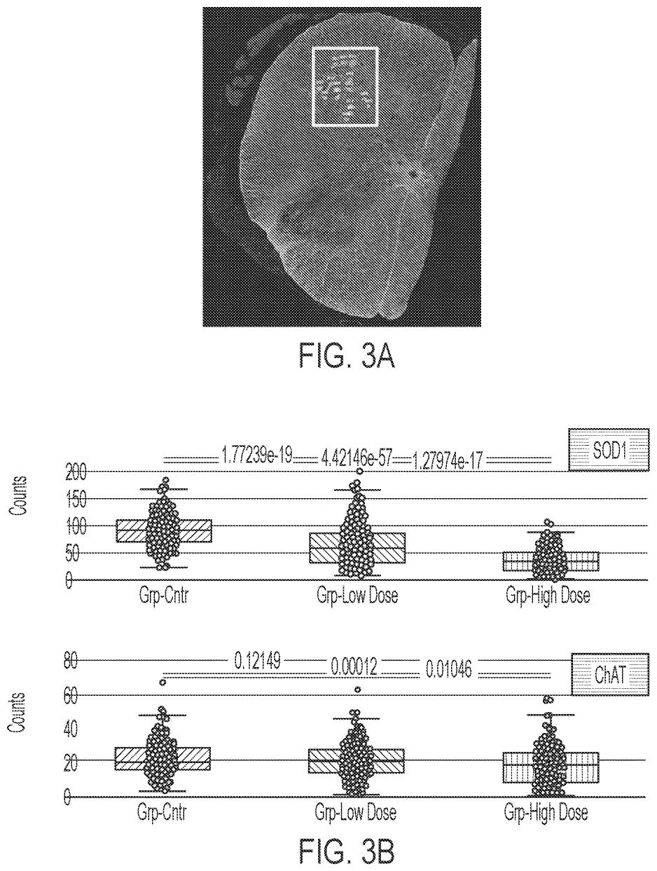

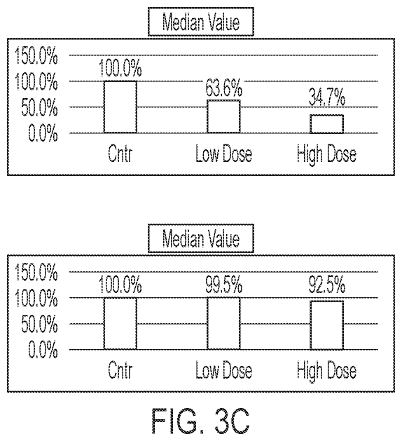

LCM of motor neurons (FIGS. 1A-1D), ISH (FIG. 2), and DSP of ChAT-positive motor neurons (FIGS. 3A-3C) demonstrated sensitive and specific knockdown of SOD1 mRNA with near single cell resolution, given the large size of these neurons. These findings in NHPs support the use of ICM AAV RNAi gene therapy targeting SOD1 as a potential approach for the treatment of SOD1-ALS.

### Example 7: SOD1 mRNA Knockdown by Intravenous Administration of AAV

In this Example, primary endpoints (body weight, neurological scoring, survival, functional endpoints, and immunohistochemistry) following SOD1 mRNA knockdown by intravenous delivery of AAV were assessed in a SOD1^{G93A} transgenic mouse ALS model. This mouse model expresses a large amount of mutant SOD1 and exhibits adult-onset neurodegeneration of spinal motor neurons and progressive motor deficits leading to paralysis.

Briefly, B6.Cg-Tg(SOD1^{G93A})1Gur mice and wild-type non-carrier mice aged about 52 days were balanced for sex, age, copy number, and body weight. Mice were intravenously administered an SOD1 miRNA-encoding AAV particle with a VOY101 capsid and a viral genome (SEQ ID NO: 109, Table 15) encoding the SOD1 targeting modulatory polynucleotide miR104-788.2 (SEQ ID NO: 2562, Table 14) under the control of an H1 promoter (AAV_VOY101.SOD1). To assess vector distribution and pharmacodynamic effects of SOD1 miRNA, upper and lower regions of the cervical, thoracic, and lumbar spinal cord were harvested at necropsy, frozen in liquid nitrogen, and stored at −80° C. until use. The lower region of each segment was homogenized, aliquoted, and both whole cell DNA and total RNA were independently isolated from the same homogenate. The experimental design is summarized in Table 28.

TABLE 28
Experimental design.

| | | | | Dose | Volume | | |
|---|---|---|---|---|---|---|---|
| Group | Genotype | Test article | Route | (VG/kg) | (ul) | N | Readout |
| WT-V | WT-B6SJL mice (B6.Cg- | Vehicle | IV | 0 | 100 | 10M/10F | Body weight, |
| | Tg(SOD1^{G93A})1Gur non- | | | | | | survival, motor |
| | carriers) | | | | | | endpoints, IHC |
| V | B6.Cg-Tg(SOD1^{G93A})1Gur | Vehicle | IV | 0 | 100 | 12M/12F | |
| C | B6.Cg-Tg(SOD1^{G93A})1Gur | AAV_VOY1 | IV | 2E13 | 100 | 12M/12F | |
| | | 01.SOD1 | | | | | |
| A | B6.Cg-Tg(SOD1^{G93A})1Gur | AAV_VOY1 | IV | 6.3E12 | 100 | 12M/12F | |
| | | 01.SOD1 | | | | | |
| B | B6.Cg-Tg(SOD1^{G93A})1Gur | AAV_VOY1 | IV | 2E12 | 100 | 12M/12F | |
| | | 01.SOD1 | | | | | |

### Spinal Cord Vector Genome Distribution

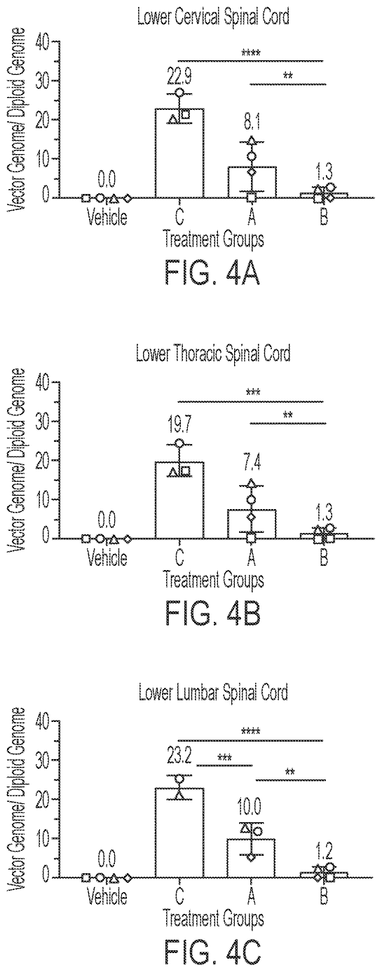

Distribution of the SOD1 miRNA AAV particle after intravenous administration in SOD1^{G93A} transgenic mice was assessed using a multiplex ddPCR assay against the transgene and host targets. As shown in FIGS. 4A-4C, nearly identical vector distribution was observed for each treatment group in all spinal cord segments assessed (lower cervical, lower thoracic, and lower lumbar spinal cord). There was a 2-fold difference in mean vector distribution between the 2E13 and 6.3E12 vg/kg groups (Groups C and A) in all spinal cord segments, and a significant difference between the 2E13 and 2E12 vg/kg Groups (Groups C and B; 1-way ANOVA/Tukey's multiple comparisons). The difference in mean vector distribution between the 6.3E12 and 2E12 vg/kg groups (Groups A and B) was at least 6-fold.

SOD1 mRNA Reduction in Spinal Cord

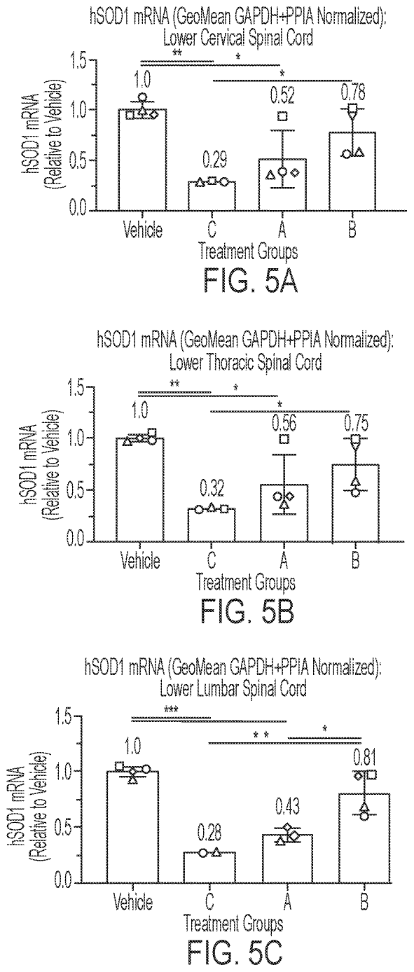

FIGS. 5A-5C show the amount of human SOD1 mRNA reduction in the spinal cord of SOD1^{G93A} transgenic mice after intravenous administration of the SOD1 miRNA AAV particle. A dose-dependent hSOD1 mRNA reduction was observed for the range of dosages tested (2E12 to 2E13 vg/kg). There was a significant decrease in hSOD1 knockdown between the 2E13 and 6.3E12 vg/kg groups (Groups C and A, respectively) and the control group (V) in all spinal cord regions tested, and between the 2E13 and 2E12 vg/kg groups (Groups C and B, respectively; 1-way ANOVA and Tukey's multiple comparisons). The amount of knockdown was about 70% in the 2E13 vg/kg group (Group C), about 50% in the 6.3E12 vg/kg group (Group A), and about 20% in the 2E12 vg/kg group (Group B), with a similar amount of reduction in hSOD1 mRNA for each group across spinal cord regions.

Efficacy of SOD1 miRNA in Mouse ALS model

Effects of the SOD1 miRNA AAV particle delivered intravenously on various features of the SOD1^{G93A} transgenic mouse model were tested.

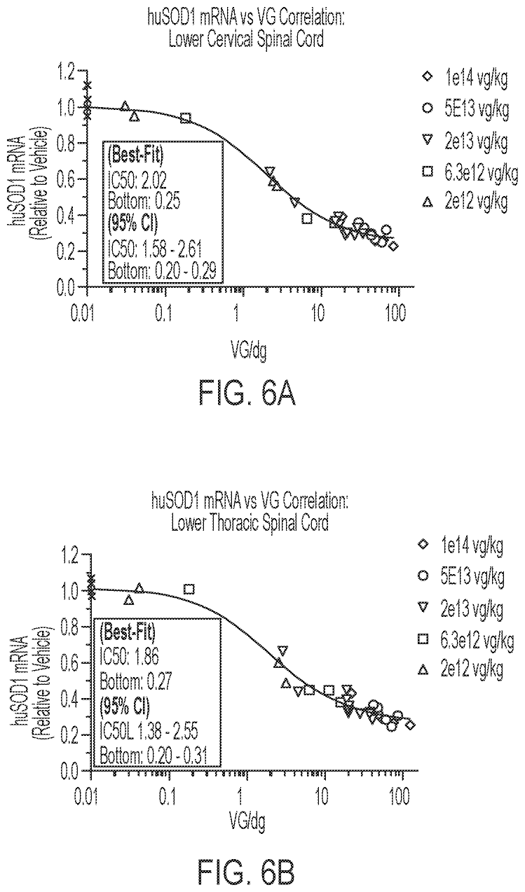

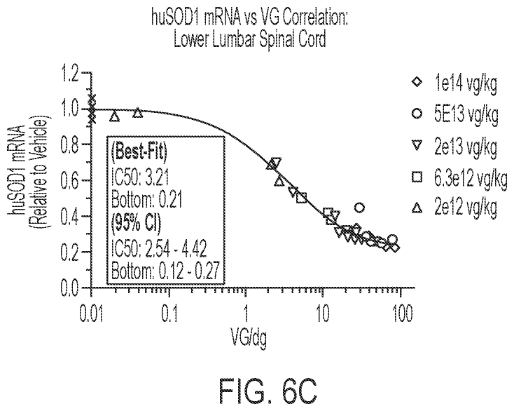

As shown in FIGS. 6A-6C, there was a clear correlation between SOD1 mRNA level and vector distribution over 2 logs of administered vector for all three spinal cord regions assessed. About 75% knockdown was observed in the cervical and thoracic spinal cord (slightly higher than in the lumbar spinal cord), with an IC50 (˜62.5% KD) of about 2 vg/dg based on a 4-parameter nonlinear curve fit (IC50 lower cervical: 2.01 vg/dg, range: 1.58-2.61 vg/dg; IC50 lower thoracic: 1.86 vg/dg, range: 1.38-2.55 vg/dg); IC50 lower lumbar: 3.21 vg/dg, range: 2.54-4.42 vg/dg).

Functional outcomes were also assessed in SOD1^{G93A} mice intravenously administered the SOD1 miRNA AAV particle.

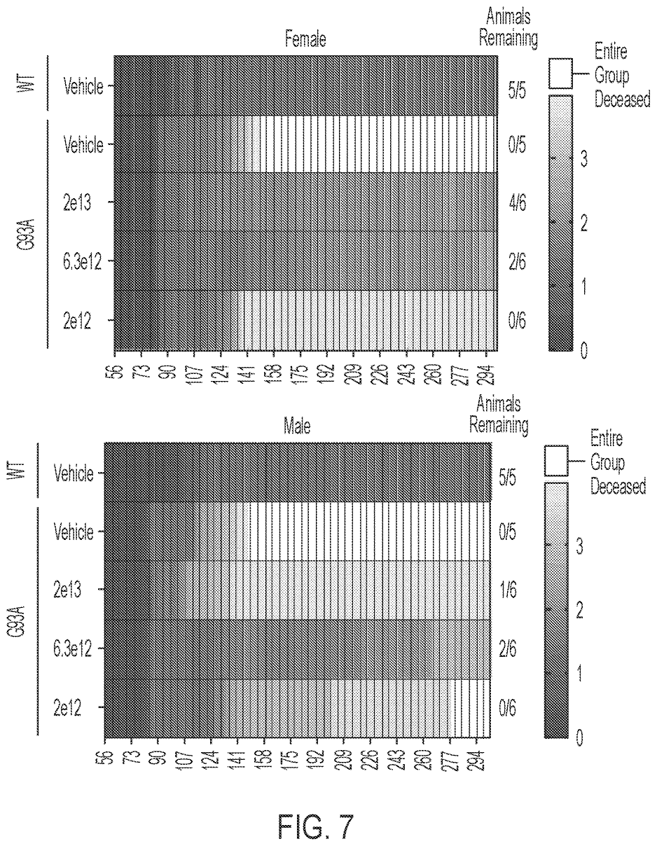

Neuroscore composite rating assessments were performed on female and male SOD1^{G93A} mice. This protocol encompassing a simple neurological scoring system designed to assess motor performance (including circling, clasping, motility, general condition, righting reflex, paw placement, contralateral reflex, visual forepaw reaching, and contralateral rotation), with a scale ranging from 0-4, with 0=no deficit, 1=first symptoms, 2=onset of paresis, 3=paralysis, and 4=human endpoint. A dose-dependent improvement was observed in Neuroscore composite rating in both males and females, although the dose-dependency was more pronounced in female mice (FIG. 7). Male mice may have exhibited a more severe phenotype and progressed pathology at the time of AAV particle administration, resulting in heightened sensitivity to SOD1 miRNA expression and a bell-shaped efficacy curve.

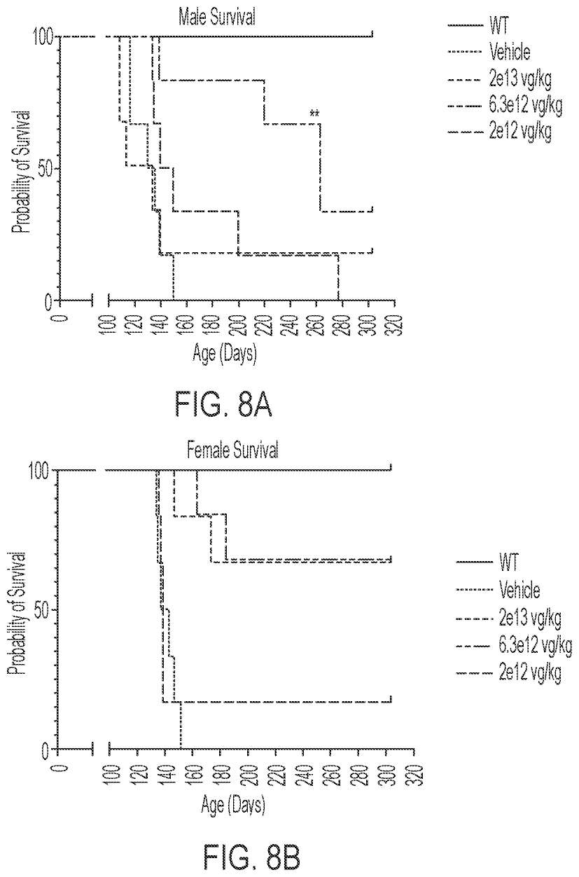

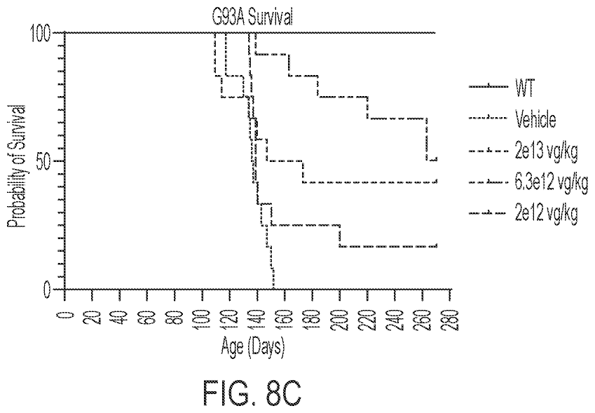

Similarly, improvement in survival was observed in female, male, and all SOD1^{G93A} mice intravenously administered the SOD1 miRNA AAV particle (FIGS. 8A-8C). In female mice, median survival was 140 days for the vehicle-treated group and 139 days for the 2E12 vg/kg group. Median survival for the 6.3E12 vg/kg and 2E13 vg/kg groups could not be calculated at age 300 days due to >50% of the cohort remaining at this time point. There were surviving female mice for all doses tested at age day 300, with the 6.3E12 vg/kg group and 2E13 vg/kg group showing a similar higher probability of survival than the 2E12 vg/kg group (FIG. 8A). In male mice, median survival was 133 days for the vehicle-treated group, 124 (n.s.) and 145 (n.s.) days for the 2E13 vg/kg and 2E12 vg/kg groups, respectively, and 263 (**p=0.003) days for the 6.3E12 vg/kg group. In males, the 6.3E12 vg/kg group showed the greatest number of surviving mice at age 300 days, followed by the 2E13 vg/kg group, whereas all 2E12 vg/kg mice had died by about age 280 days (FIG. 8B). Statistical analysis was performed using the log-rank (Mantel-Cox) test. When combining the data for both males and females, while vehicle-administered SOD1^{G93A} mice died before age 160 days, surviving mice were observed for all SOD1 miRNA AAV particle groups, with the largest number of mice surviving at age 280 days in the 6.3E12 vg/kg group. The least number of surviving mice was observed with the 2E12 vg/kg group at age 280 days (FIG. 8C).

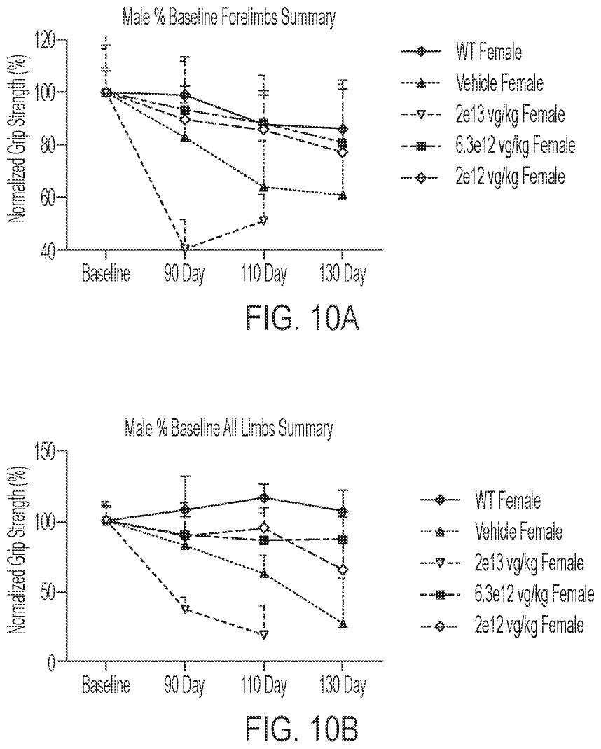

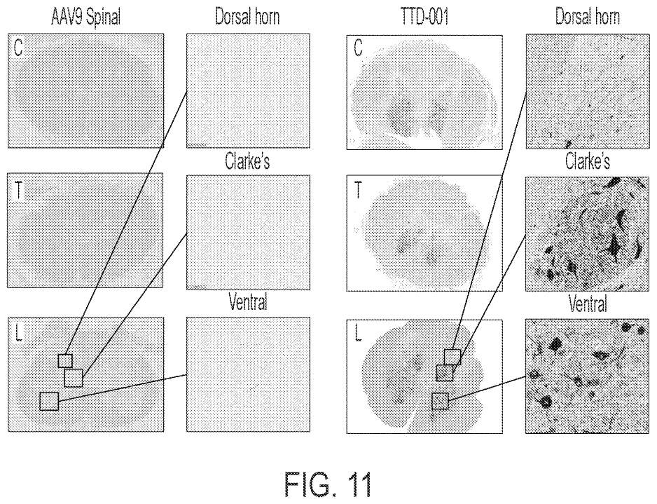

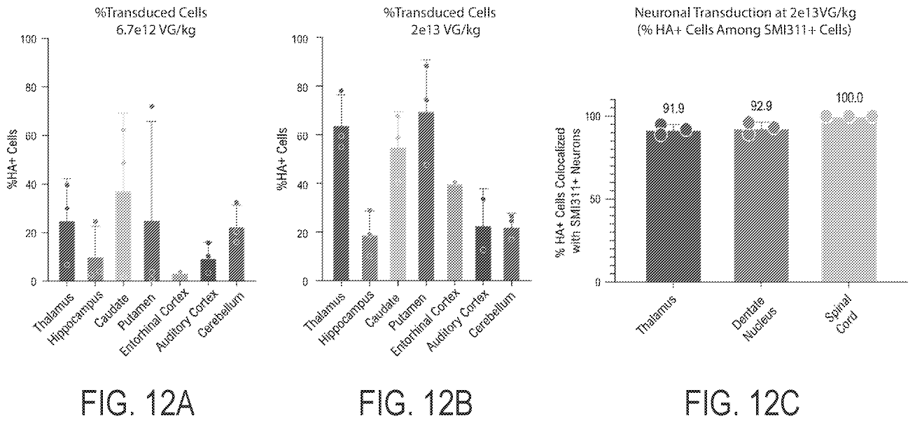

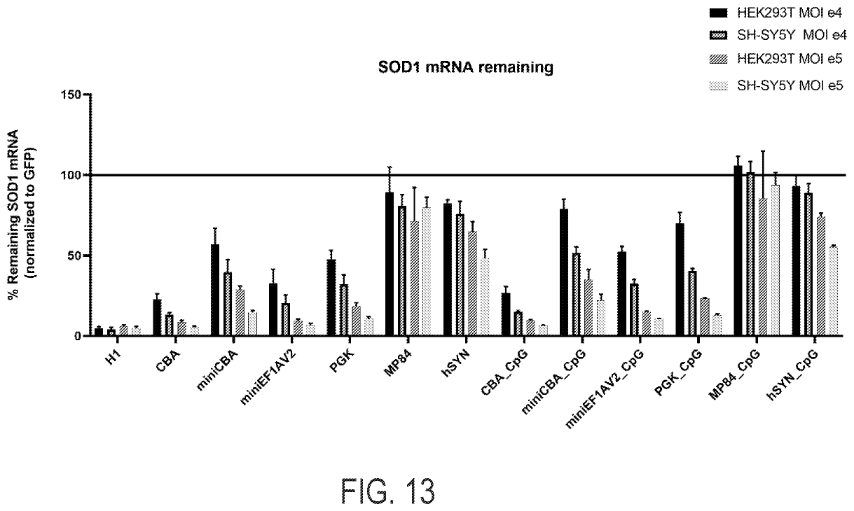

Grip strength was also assessed (FIGS. 9A, 9B, 10A, 10B). Grip strength provides a measure of forepaw and hindlimb muscle strength by measuring the forced required to pull the mouse off a narrow bar or grid. The apparatus used to test grip strength consists of a grasping bar connected to a force transducer. Each animal underwent five trials, and the mean value was calculated. Mice that are no longer able to grab the bar or grid due to disease progression were removed from the experiment, as was the case in the 2E13 vg/kg treatment group in both males and females. In both female and male SOD1^{G93A} mice, the intermediate dose of 6.3E12 vg/kg had the strongest effect of maintaining near wild-type levels of grip strength for both forelimb and all limbs combined at day 130 of age. In female mice, the 2E12 vg/kg dose showed no improvement in grip strength, with a trend similar to vehicle administered mice, while in male mice, a clear improvement in grip strength was observed at this dose. These data are consistent with other behavioral findings, suggesting female mice are more responsive to higher doses of AAV particle, while male mice are more susceptible to overexpression of SOD1 miRNA and do not tolerate the highest dose (2E13 vg/kg).

Overall, these studies demonstrated robust SOD1 knockdown throughout the rostral-caudal extent of the spinal cord that correlated with vector genome levels and significant improvements in motor performance and survival extension. These results suggest that the combination of potent and tolerable AAV miRNA mediated knockdown with intravenous dosing of a BBB-penetrant capsid demonstrates substantial phenotypic rescue in an SOD1-ALS mouse model.

### Example 8: SOD1 mRNA Knockdown by SOD1 miRNA Expressed Under Different Promoters

In this example, SOD1 mRNA knockdown was assessed by measuring the remaining SOD1 mRNA in HEK293T and SH-SY5Y cell lines. Briefly, HEK293T and SH-SY5Y cells were plated at 15,000 cell density in 96-well plate. Next day, cells were treated with SOD1 miRNA-encoding AAV particle with a AAV2 capsid and a viral genome (Table 16 and 17) encoding the SOD1 targeting modulatory polynucleotide miR104-788.2 (SEQ ID NO: 2562, Table 14) under the control of an H1 promoter (AAV_VOY101.SOD1) (Table 16) or other Pol II promoters (CBA, miniCBA, miniEF1AV2, PGK, MP84, hSYN, CBA_CpG depleted exon, miniCBA_CpG depleted exon, miniEF1AV2_CpGdepleted exon, PGK_CpG depleted exon, MP84_CpGdepleted exon and hSYN_CpG depleted exon, the sequences are included in Table 17). Two days after AAV particle treatment, the cells were harvested and total RNA were extracted to measure the SOD1 mRNA levels by qRT-PCR. We observed a dose dependent SOD1 mRNA knockdown (KD) in both HEK293T and SH-SY5Y cells. SOD1 KD was the highest for miRNAs that were expressed under H1 (Pol III) promoter with almost 95% KD at both MOIs tested. For the Pol II promoters that we tested, the KD levels were the following: CBA=CBA_CpG depleted exon>miniEF1AV2=miniEF1AV2_CpGdepleted exon>PGK=PGK_CpG depleted exon>miniCBA=miniCBA_CpGdepleted exon>hSYN=hSYN_CpG depleted exon>MP84=MP84_CpG depleted. A significant difference was not observed between the transgenes that has full CpG amount and decreased CpG amount (for the transgenes that have depleted CpG in exonic regions of the transgenes).

### Example 9 SOD1 mRNA Knockdown by Intravenous Administration of AAV

In this example, primary endpoints (vector genome tissue distribution, vector genome cellular tropism, and SOD1 mRNA reduction in various tissues) will be assessed following intravenous administration of the AAV capsid variants described herein. Briefly, cynomolgus macaques (Macaca fascicularis) aged 2-5 years and weighing 2-4 kg will be used for the study. Approximately three weeks before the dosing date, 1 ml of blood will be collected for serum preparation from a sufficient number of animals with normal physical examinations, clinical chemistries, and complete blood counts (CBCs) for a neutralizing antibody (Nab) screening using standard protocols. The serologically qualified animals will be assigned to the study. Animals will be randomized across low (1×1012 vg/kg), mid (3×1012 vg/kg), and high (1×1013 vg/kg) dose groups for each capsid variant, as well as a vehicle control. Each subject will receive a single intravenous infusion via saphenous or cephalic vein using an infusion pump on Day 0, with a target infusion rate of 5 ml/kg/hour. A selected group of tissues will be harvested on day 28 post AAV administration, with special attention being taken for the processing of the brain and spinal cord. To assess vector distribution and pharmacodynamic effects of SOD1 miRNA, cervical, thoracic, and lumbar spinal cord sections will be harvested. To assess cellular tropism of the miRNA, the even numbered cervical, thoracic, and lumbar spinal cord sections will be harvested. Additional CNS regions/tissues will be assessed (e.g., motor cortex and brainstem), along with an extensive list of peripheral tissues. Distribution of the SOD1 miRNA AAV particle will be assessed using a multiplex ddPCR assay against the transgene and host targets. In situ hybridization will be utilized to detect cellular distribution of the miRNA and resulting SOD1 reduction.

### XI. Equivalents and Scope

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. An isolated, e.g., recombinant, AAV particle comprising an AAV capsid variant and a nucleic acid comprising a transgene encoding a SOD1 targeting polynucleotide wherein
(A) the SOD1 targeting polynucleotide is a single-stranded antisense RNA molecule, a single-stranded siRNA, or a double-stranded RNA (e.g., a siRNA duplex), which comprises an antisense strand comprising a region that is substantially complementary (e.g., at least 70%, 75%, 80%, 85%, 90%, 95%, 99% or 100% complementary) to at least part of an mRNA transcript of a human SOD1 gene (e.g., a human wild-type SOD gene or a human mutated SOD gene), and wherein the SOD1 targeting polynucleotide inhibits expression of the human SOD1 gene; and
(B) the AAV capsid variant comprises an amino sequence comprising the following formula: [N1]-[N2], wherein:
(i) [N1] comprises X1, X2, X3, X4, and X5, wherein:
(a) position X1 is: P, Q, A, H, K, L, R, S, or T;
(b) position X2 is: L, I, V, H, or R;
(c) position X3 is: N, D, I, K, or Y;
(d) position X4 is: G, A, C, R, or S; and
(e) position X5 is: A, S, T, G, C, D, N, Q, V, or Y; and
(ii) [N2] comprises the amino acid sequence of VHLY, VHIY, VHVY, or VHHY; and/or a substitution, e.g., a conservative substitution, of any of the aforesaid amino acids in (i) and/or (ii);
wherein [N1]-[N2] is present from N-terminus to C-terminus, immediately subsequent to position 586, numbered according to any one of SEQ ID NO: 5, 8, 138, or 3636;
optionally, wherein the AAV capsid variant comprises:
(a) an amino acid sequence at least 95% identical to the amino acid sequence of positions 203-743, e.g., a VP3, of any one of SEQ ID NO: 5, 8, or 3636; or
(b) an amino acid sequence at least 95% identical to the amino acid sequence of positions 203-736, e.g., a VP3, of SEQ ID NO: 138.

2. The isolated AAV particle of claim 1, wherein [N1] comprises: PLNGA (SEQ ID NO: 3679), SLNGA (SEQ ID NO: 3702), QLNGA (SEQ ID NO: 3703), ALNGA (SEQ ID NO: 3704), PLNGS (SEQ ID NO: 3705), PVNGA (SEQ ID NO: 3706), PLNGG (SEQ ID NO: 3707), PLNGT (SEQ ID NO: 3708), PLDGA (SEQ ID NO: 3709), QLNGS (SEQ ID NO: 3710), PLNGN (SEQ ID NO: 3711), SLDGA (SEQ ID NO: 3712), HLNGA (SEQ ID NO: 3713), ALNGT (SEQ ID NO: 3714), PINGA (SEQ ID NO: 3715), ALDGA (SEQ ID NO: 3716), PLNCA (SEQ ID NO: 3717), PLNGQ (SEQ ID NO: 3718), PLDSA (SEQ ID NO: 3719), RLDGA (SEQ ID NO: 3720), QLNGN (SEQ ID NO: 3721), PLNGY (SEQ ID NO: 3722), PLDSS (SEQ ID NO: 3723), PLNGC (SEQ ID NO: 3724), PLYGA (SEQ ID NO: 3725), TLNGA (SEQ ID NO: 3726), PVDGA (SEQ ID NO: 3727), PLKGA (SEQ ID NO: 3728), PLNGD (SEQ ID NO: 3729), KLDGA (SEQ ID NO: 3730), PHNGA (SEQ ID NO: 3731), PLNGV (SEQ ID NO: 3732), PLNAA (SEQ ID NO: 3733), QLNGY (SEQ ID NO: 3734), PLDGS (SEQ ID NO: 3735), LLNGA (SEQ ID NO: 3736), PLNRA (SEQ ID NO: 3737), PLIGA (SEQ ID NO: 3738), PRNGA (SEQ ID NO: 3739), or ALNGS (SEQ ID NO: 3740), or a substitution, e.g., a conservative substitution, of any of the aforesaid amino acid sequences.

3. The isolated AAV particle of claim 1 or claim 2 wherein [N1]-[N2] comprises:
(i)
(SEQ ID NO: 3648)
PLNGAVHLY,
(SEQ ID NO: 3795)
ALDGAVHLY,
(SEQ ID NO: 3704)
ALNGAVHLY,
(SEQ ID NO: 3796)
PINGAVHLY,
(SEQ ID NO: 3797)
PLDGAVHLY,
(SEQ ID NO: 3798)
PLDSAVHLY,
(SEQ ID NO: 3799)
PLDSSVHLY,
(SEQ ID NO: 3800)
PLNGGVHLY,
(SEQ ID NO: 3801)
PLNGNVHLY,
(SEQ ID NO: 3802)
PLNGSVHLY,
(SEQ ID NO: 3803)
PLNGTVHLY,
(SEQ ID NO: 3804)
QLNGAVHLY,
(SEQ ID NO: 3805)
SLDGAVHLY,
(SEQ ID NO: 3806)
SLNGAVHLY,
(SEQ ID NO: 3807)
TLNGAVHLY,
(SEQ ID NO: 3808)
PLNGAVHIY,
(SEQ ID NO: 3809)
PLDGAVHVY,
(SEQ ID NO: 3810)
PLNGAVHHY;
(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising at least one, two, or three but no more than four modifications, e.g., substitutions (e.g., conservative substitutions), insertions, or deletions, relative to any of the amino acid sequences in (i); or
(iv) an amino acid sequence comprising at least one, two, or three but no more than four different amino acids, relative to any one of the amino acid sequences in (i).

4. The isolated AAV particle of any one of claims 1-3, wherein the AAV capsid variant further comprises [N3], wherein [N3] comprises X6, X7, X8, and X9, wherein:
(a) position X6 is: A, D, S, or T;
(b) position X7 is: Q, K, H, L, P, or R;
(c) position X8 is: A, P, E, or R; and
(d) position X9 is: Q, H, K, or P; and/or
an amino acid modification, e.g., a conservative substitution, of any of the aforesaid amino acids in (a)-(d).

5. The isolated AAV particle of claim 4, wherein [N3] comprises AQAQ (SEQ ID NO: 3759), SQAQ (SEQ ID NO: 3760), AQPQ (SEQ ID NO: 3761), AQSQ (SEQ ID NO: 3762), AKAQ (SEQ ID NO: 3763), AHAQ (SEQ ID NO: 3764), AQAP (SEQ ID NO: 3765), DQAQ (SEQ ID NO: 3766), APAQ (SEQ ID NO: 3767), AQAK (SEQ ID NO: 3768), AQAH (SEQ ID NO: 3769), AQEQ (SEQ ID NO: 3770), ALAQ (SEQ ID NO: 3771), ARAQ SEQ ID NO: 3772), or TQAQ (SEQ ID NO: 3773), or a substitution, e.g., a conservative substitution, of any of the aforesaid amino acid sequences

6. The isolated AAV particle of any one claims 1-5, wherein the AAV capsid variant further comprises [N4], wherein [N4] comprises X10, X11, and X12, wherein:
(a) position X10 is: L, T, V, R, S, A, C, I, K, M, N, P, or Q;
(b) position X1I is: G, S, A, T, M, V, Q, L, H, I, K, N, P, R, or Y; and
(c) position X12 is: W, S, P, G, A, Q, L, M, K, C, E, F, H, R, T, V, or Y; and/or
an amino acid modification, e.g., a conservative substitution, of any of the aforesaid amino acids in (a)-(c).

7. The isolated AAV particle of claim 6, wherein [N4] is or comprises LSP, TGW, TGL, TGS, TGG, TAW, TGR, TAS, LSS, TSS, SSL, SSS, TLS, TVS, VSS, TSP, VSP, TMS, VAS, TAL, TTS, TLP, VLP, RGW, LSG, LAS, SSP, LLP, STS, TSA, TTP, SAL, LGS, VTP, VSA, IGW, TGF, LTP, TLA, LSA, TVG, TAP, TMP, TSL, VQS, SSM, SLP, VSQ, RSS, TST, VMS, TTA, TQP, LST, LAP, TVA, RLS, TGY, TSG, TAG, VMP, TSQ, TMA, VGS, TSW, TGV, TGT, TLG, LMP, VQP, TGM, SMS, SQL, IGS, RSV, TAA, STP, LSQ, TAQ, TGP, ASP, VSG, SAP, TLQ, LQP, TAT, TGQ, ATS, IGG, VAA, TSM, TVW, TAM, TGA, VAT, QSP, TQA, VQA, RSP, LAT, VAQ, LAA, RST, RTL, LGT, LMS, LGP, RTS, SQP, VLG, SVS, TMQ, SAV, LAG, SGP, TNS, RLT, TTQ, SAA, TSV, RLG, RAS, STQ, CSP, SAG, ALP, VTS, ISP, SVG, LTS, TTT, RSG, TQL, LNP, TVQ, IAS, LAQ, LSR, LSN, TTG, TSN, SMA, TKS, SVA, TQQ, VQQ, RLP, SAM, TAV, TQW, SSR, TQT, VNS, RSA, LMG, RQS, LVG, VTA, RTT, SMG, VMA, TKP, SAQ, NSP, ATP, VAG, RGS, VKP, RMS, NLP, NAL, RTP, RQL, VQG, VTG, VST, NAS, RVE, ATG, AMS, RNS, VMQ, SMQ, LQQ, TMG, LGQ, TSH, AAP, RSQ, TYS, ITP, VAK, TQM, TKA, SQQ, ISG, VSR, RTA, RML, SQM, VAN, CTP, ISS, AGP, TAK, RTG, LHP, TMT, AQP, QAP, RQP, LKS, NTT, TSK, RYS, KSS, NTP, VGG, IAA, LMA, MAP, VHP, VLS, LAN, ATQ, TNA, TAN, VSN, AAA, AVG, LTA, SAN, RAG, RQG, TLR, LSH, SAF, RAA, IQP, ILG, VNG, SVQ, LSK, TNG, RTQ, TMN, RGG, TTR, VRP, VKA, LAR, NQP, TMK, TYA, TQK, TTK, IAG, TQN, LAH, NTQ, RQQ, RAQ, TKQ, TQH, TNQ, LMQ, VNA, VQT, TQR, VGK, VKQ, IQS, LQR, TMM, VGN, RIG, SAK, RIA, VQN, NVQ, RIP, NAQ, NMQ, TPS, LTN, VTK, PGW, LPP, SPP, TPA, TGC, VPP, TPT, TPW, TPP, RPP, TPQ, TPR, TPG, VPA, VPQ, RPG, KGW, TRW, TAR, IPP, RSL, LVP, KGS, VAP, KGG, KAW, PGS, TRL, or AGW, or a substitution, e.g., a conservative substitution, of any of the aforesaid amino acid sequences;
(ii) LSP; or
(iii) TGW.

8. The AAV particle of any one of claims 1-7, wherein the AAV capsid variant further comprises [N5], wherein [N5] comprises X13, X14, and X15, wherein:
(a) position X13 is: V, D, F, G, L, A, E, or I;
(b) position X14 is: Q, K, R, H, E, L, or P; and
(c) position X15 is: N, T, K, H, D, Y, S, I, or P; and/or
an amino acid modification, e.g., a conservative substitution, of any of the aforesaid amino acids in (a)-(c).

9. The AAV particle of claim 8, wherein [N5] is or comprises VQN, VKN, VQT, VQK, DQN, VQH, GQN, VQI, VHN, FQN, LQN, VLN, VRN, VQS, VQY, AQN, VEN, VQD, VPN, IQN, VKK, DKN, VKT, VQP, EQN, GQT, FQK, GHN, or VPH, or a substitution, e.g., a conservative substitution, of any of the aforesaid amino acid sequences;
(ii) VKN, VPN, VEN, or VHN; or
(iii) VQN.

10. The isolated AAV particle of claim 8 or 9, wherein [N1]-[N2]-[N3]-[N4]-[N5] comprises:
(i) the amino acid sequence of any of SEQ ID NOs: 139-1138;
(ii) the amino acid sequence of PLNGAVHLYAQAQLSPVKN (SEQ ID NO: 566);
(iii) the amino acid sequence of PLNGAVHLYAQAQTGWVPN (SEQ ID NO: 314);
(iv) the amino acid sequence of PLNGAVHLY (SEQ ID NO: 3648);
(v) the amino acid sequence of any of SEQ ID NOs: 14-17, 40-136, 314, 325, 491, 499, 529, 558, 566, 576, 603, 610, 625, 631, 648, 649, 700, 703, 720, 755, 763, 765, 771, 791, 804, 816, 818, 819, 828, 859, 864, 871, 885, 946, 960, 966, 978, 979, 1016, 1033, 1032, 1037, 1058, 1081, 1100, 1122, or 1174-1193;
(v) an amino acid sequence comprising any portion of an amino acid sequence in (i)-(v), e.g., any 2, 3, 4, 5, 6, 7, 8, 9, 10, 11, 12, 13, 14, 15, 16, 17, or 18 amino acids, e.g., consecutive amino acids, thereof;
(vi) an amino acid sequence comprising at least one, two, or three but no more than four substitutions (e.g., conservative substitutions), insertions, or deletions, relative to any of the amino acid sequences in (i)-(v); or
(vii) an amino acid sequence comprising at least one, two, or three but no more than four different amino acids, relative to any one of the amino acid sequences in (i)-(v).

11. The isolated AAV particle of any one of claims 8-10, wherein the AAV capsid variant comprises from N-terminus to C-terminus [N1]-[N2]-[N3]-[N4]-[N5], wherein:
(i) [N1] is present immediately subsequent to position 586, and replaces positions 587 and 588 (e.g., A587 and Q588), numbered according to SEQ ID NO: 138;
(ii) [N2] is present immediately subsequent to [N1];
(iii) [N3] is present immediately subsequent to position 588, and replaces positions 589-592 (e.g., A589, Q590, A591, Q592), numbered according to the amino acid sequence of SEQ ID NO: 138;
(iv) [N4] is present immediately subsequent to position 592, and replaces positions 593-595 (e.g., T593, G594, W595), numbered according to the amino acid sequence of SEQ ID NO: 138;
(v) [N5] is present immediately subsequent to position 595, and replaces positions 596-598 (e.g., V596, Q597, N598), numbered according to the amino acid sequence of SEQ ID NO: 138; and/or
(vi) [N1]-[N2]-[N3]-[N4]-[N5] is present immediately subsequent to position 586 and replaces positions 587-598 (e.g., A587, Q588, A589, Q590, A591, Q592, T593, G594, W595, V596, Q597, N598), numbered according to SEQ ID NO: 138.

12. The isolated AAV particle of any one of claims 8-11, wherein:
(i) the AAV capsid variant comprises an amino acid other than A at position 587 and/or an amino acid other than Q at position 588, numbered according to SEQ ID NO: 138;
(ii) [N1] corresponds to positions 587-591 of SEQ ID NO: 5, 8, or 3636;
(iii) [N2] corresponds to positions 592 to 595 of SEQ ID NO: 5, 8, or 3636;
(iv) [N3] corresponds to positions 596-599 of SEQ ID NO: 5, 8, or 3636;
(v) [N4] corresponds to positions 600-602 of SEQ ID NO: 5, 8, or 3636;
(vi) [N5] corresponds to positions 603-605 of SEQ ID NO: 5, 8, or 3636; and/or
(vii) [N1]-[N2]-[N3]-[N4]-[N5] corresponds to positions 587-605 of SEQ ID NO: 5, 8, or 3636.

13. An isolated, e.g., recombinant, AAV particle comprising an AAV capsid variant and a nucleic acid comprising a transgene encoding a SOD1 targeting polynucleotide (e.g., a SOD1 targeting RNAi), wherein,
(A) the SOD1 targeting polynucleotide is a single-stranded antisense RNA molecule, a single-stranded siRNA, or a double-stranded RNA (e.g., a siRNA duplex), which comprises an antisense strand comprising a region that is substantially complementary (e.g., at least 70%, 75%, 80%, 85%, 90%, 95%, 99% or 100% complementary) to at least part of an mRNA transcript of a human SOD1 gene (e.g., a human wild-type SOD gene or a human mutated SOD gene), and wherein the SOD1 targeting polynucleotide inhibits expression of the human SOD1 gene; and
(B) the AAV capsid variant comprises [A][B], wherein [A] comprises the amino acid sequence of PLNGA (SEQ ID NO: 3679), and [B] comprises X1, X2, X3, X4, wherein:
(i) X1 is: V, I, L, A, F, D, or G;
(ii) X2 is: H, N, Q, P, D, L, R, or Y;
(iii) X3 is: L, H, I, R, or V; and
(iv) X4 is Y; and/or
wherein the AAV capsid variant comprises an amino acid modification, e.g., a conservative substitution, of any of the aforesaid amino acids in (i)-(iv);
wherein [A][B] is present from N-terminus to C-terminus, immediately subsequent to position 586, numbered according to any one of SEQ ID NO: 5, 8, 138, or 3636;
optionally wherein the AAV capsid variant comprises:
(a) an amino acid sequence at least 95% identical to the amino acid sequence of positions 203-743, e.g., a VP3, of any one of SEQ ID NO: 5, 8, or 3636; or
(b) an amino acid sequence at least 95% identical to the amino acid sequence of positions 203-736, e.g., a VP3, of SEQ ID NO: 138.

14. The isolated AAV particle of claim 13, wherein [B] is or comprises:
(i) VHLY (SEQ ID NO: 3741), VHHY (SEQ ID NO: 3747), VHIY (SEQ ID NO: 3746), VNLY (SEQ ID NO: 3744), VQLY (SEQ ID NO: 3751), IHLY(SEQ ID NO: 3752), LHLY (SEQ ID NO: 3749), VPLY(SEQ ID NO: 3743), VDLY (SEQ ID NO: 3753), AHLY (SEQ ID NO: 3754), VHRY (SEQ ID NO: 3745), FHLY (SEQ ID NO: 3748), DHLY (SEQ ID NO: 3750), VLLY (SEQ ID NO: 3755), GHLY (SEQ ID NO: 3756), VRLY (SEQ ID NO: 3757), VHVY (SEQ ID NO: 3742), or VYLY (SEQ ID NO: 3758) or a substitution, e.g., a conservative substitution, of any of the aforesaid amino acid sequences; or
(ii) VHLY (SEQ ID NO: 3741).

15. The isolated AAV particle of claim 13 or claim 14, wherein [A][B] comprises:
(i) PLNGAVH (SEQ ID NO: 3681), PLNGAVN (SEQ ID NO: 1254), PLNGAVQ (SEQ ID NO: 1255), PLNGAIH (SEQ ID NO: 1256), PLNGALH (SEQ ID NO: 1257), PLNGAVP (SEQ ID NO: 1258), PLNGAVD (SEQ ID NO: 1259), PLNGAAH (SEQ ID NO: 1260), PLNGAFH (SEQ ID NO: 1261), PLNGADH (SEQ ID NO: 1263), PLNGAVL (SEQ ID NO: 1264), PLNGAGH (SEQ ID NO: 1265), PLNGAVR (SEQ ID NO: 1266), or PLNGAVY (SEQ ID NO: 1267); or
(ii) PLNGAVH (SEQ ID NO: 3681);
(iii) amino acid sequence comprising any portion of an amino acid sequence in (i) or (ii), e.g., any 2, 3, 4, 5, or 6 amino acids, e.g., consecutive amino acids, thereof;
(iv) an amino acid sequence comprising at least one, two, or three but no more than four modifications, e.g., substitutions (e.g., conservative substitutions), insertions, or deletions, relative to any of the amino acid sequences in (i) or (ii); or
(v) an amino acid sequence comprising at least one, two, or three but no more than four different amino acids, relative to any one of the amino acid sequences in (i) or (ii).

16. The isolated AAV particle of any one of claims 13-15, wherein the AAV capsid variant further comprises [C], wherein [C] comprises X4, X5, X6, and X7, wherein:
(a) position X4 is: A, D, S, or T;
(b) position X5 is: Q, K, H, L, P, or R;
(c) position X6 is: A, P, or E; and
(d) position X7 is: Q, H, K, or P; and/or
an amino acid modification, e.g., a conservative substitution, of any of the aforesaid amino acids in (a)-(d).

17. The isolated AAV particle of claim 16, wherein [C] is or comprises:
(i) AQAQ (SEQ ID NO: 3759), AQPQ (SEQ ID NO: 3761), AKAQ (SEQ ID NO: 3763), DQAQ (SEQ ID NO: 3766), SQAQ (SEQ ID NO: 3760), AHAQ (SEQ ID NO: 3764), AQEQ (SEQ ID NO: 3770), AQAK (SEQ ID NO: 3768), ALAQ (SEQ ID NO: 3771), APAQ (SEQ ID NO: 3767), ARAQ SEQ ID NO: 3772), AQAH (SEQ ID NO: 3769), AQAP (SEQ ID NO: 3765), or TQAQ (SEQ ID NO: 3773), or a substitution, e.g., a conservative substitution, of any of the aforesaid amino acid sequences; or
(ii) AQAQ (SEQ ID NO: 3759).

18. The isolated AAV particle of any one of claims 1-17, wherein the AAV capsid variant further comprises:
(i) the amino acid L at position 593 numbered according to SEQ ID NO: 138 or at position 600 numbered according to SEQ ID NO: 5, 8, or 3636; the amino acid S at position 594 numbered according to SEQ ID NO: 138, or at position 601 numbered according to SEQ ID NO: 5, 8, or 3636; and the amino acid P at position 595 numbered according to SEQ ID NO: 138 or at position 602 numbered according to SEQ ID NO: 5, 8, or 3636; or
(ii) the amino acid T at position 593 numbered according to SEQ ID NO: 138 or at position 600 numbered according to SEQ ID NO: 5, 8, or 3636; the amino acid G at position 594 numbered according to SEQ ID NO: 138, or at position 601 numbered according to SEQ ID NO: 5, 8, or 3636; and the amino acid W at position 595 numbered according to SEQ ID NO: 138 or at position 602 numbered according to SEQ ID NO: 5, 8, or 3636.

19. The isolated AAV particle of any one of claims 13-18, wherein the AAV capsid variant further comprises [D], wherein [D] comprises X8, X9, and X10, wherein:
(a) position X8 is: L, T, V, S, R, I, A, N, C, Q, M, P, or K;
(b) position X9 is: S, G, T, M, A, G, K, Q, V, I, R, N, P, L, H, or Y; and
(c) position X10 is: P, W, K, Q, S, C, A, G, N, T, R, V, M, H, L, E, F, or Y; or a substitution, e.g., a conservative substitution, of any of the aforesaid amino acids in (a)-(c).

20. The isolated AAV particle of any one of claims 13-19, wherein [D] is or comprises:
(i) LSP, TGW, TTK, TMK, VAQ, TAW, TGS, VKQ, SAP, LSK, LAP, LAQ, TAK, SAK, TGC, TQK, TVA, TTQ, TAQ, RIA, RAS, TTP, LTP, STP, TSP, TMQ, TSK, VSQ, VSP, TVQ, VTA, RQP, ISG, VRP, LGP, TNQ, VQQ, VAN, AAP, RST, TMA, IQP, IAS, TVS, RGS, NSP, LQP, VTG, VMQ, SMA, VGK, IQS, CSP, LQR, TPP, VTK, SSP, AGP, LAR, TTT, TGG, TLQ, TMS, VAK, RAA, TVG, LNP, LSQ, TKP, TNA, LAT, VTP, VQA, TTS, CTP, TAG, TSQ, TMN, TST, VKP, ASP, VAA, LKS, IAA, TAA, TKA, VSN, TAP, LMP, LHP, RAQ, LTN, RTT, TSV, RMS, VGN, LMQ, TAT, VHP, ISS, VAS, TRW, TMT, RSS, RTG, VAT, VTS, VSS, TNS, VKA, SGP, TGP, TAM, TQP, TQQ, VSR, VSA, VLS, TQH, LAS, QAP, NAQ, ATP, VQP, TTA, LAA, RSG, LMA, TMP, LAN, VST, SAQ, NTP, TGL, TAV, RLG, RTL, TQM, ITP, TVW, RSA, TAS, TMG, VQS, ISP, VGG, TAL, LAG, RTA, RSP, TLA, LAH, TSL, RLS, LMG, SMQ, TQT, VGS, VSG, VMA, IGG, IAG, TGR, LSH, VQT, RNS, TLP, TKQ, LGQ, NMQ, NVQ, RGG, VMS, TTG, LSR, MAP, ILG, TGT, TSS, TSH, RIG, SAM, TSM, SMG, SMS, TSG, TGA, VNS, VAG, IGS, LGS, VNG, LTA, VQN, TKS, SVG, NAS, TSA, TAN, LTS, RSQ, RIP, RVE, VLP, SVA, LQQ, LST, SAA, RTS, TQN, VNA, LMS, TMM, RSV, TQL, RTP, RQQ, VQG, PGW, STQ, QSP, RYS, TQR, SAG, RQS, SQP, STS, VLG, NQP, LGT, RAG, TGM, LSN, RLP, RQG, RLT, TLR, SAF, SVQ, LLP, RTQ, LPP, AQP, TPQ, TSW, NTT, TTR, TQW, NTQ, TYA, TLS, NLP, ATS, ATQ, LSS, TQA, VMP, NAL, RML, RQL, TLG, TGF, SAL, SQL, LSA, TGQ, TNG, AAA, SAV, LSG, SSR, SPP, LVG, TPA, KGW, VPP, ATG, SAN, SQQ, SSM, AVG, VAP, TPS, RGW, SSL, TYS, TPT, IGW, KSS, TGY, RSL, SVS, TSN, SQM, VPA, AMS, TPG, TGV, VPQ, SLP, ALP, TPW, TPR, SSS, RPP, IPP, AGW, or RPG, or a substitution, e.g., a conservative substitution, of any of the aforesaid amino acid sequences;
(ii) LSP; or
(iii) TGW.

21. The isolated AAV particle of any one of claims 1-20, wherein the AAV capsid variant further comprises:
(i) the amino acid V at position 596 numbered according to SEQ ID NO: 138 or at position 603 numbered according to SEQ ID NO: 5, 8, or 3636; the amino acid K, P, E or H at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(ii) the amino acid V at position 596 numbered according to SEQ ID NO: 138 or at position 603 numbered according to SEQ ID NO: 5, 8, or 3636; the amino acid K at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636;
(iii) the amino acid V at position 596 numbered according to SEQ ID NO: 138 or at position 603 numbered according to SEQ ID NO: 5, 8, or 3636; the amino acid P at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636; or
(iv) the amino acid V at position 596 numbered according to SEQ ID NO: 138 or at position 603 numbered according to SEQ ID NO: 5, 8, or 3636; the amino acid Q at position 597 numbered according to SEQ ID NO: 138 or at position 604 numbered according to SEQ ID NO: 5, 8, or 3636; and the amino acid N at position 598 numbered according to the amino acid sequence of SEQ ID NO: 138 or at position 605 numbered according to SEQ ID NO: 5, 8, or 3636.

22. The isolated AAV particle of any one of claims 13-21, wherein the AAV capsid variant further comprises [E], wherein [E] comprises X11, X12, and X13, wherein:
(a) position X1I is: V, D, F, A, E, L, G, or I;
(b) position X12 is: Q, R, P, K, L, H, or E; and
(c) position X13 is: N, H, S, T, P, K, I, D, or Y; or a substitution, e.g., a conservative substitution, of any of the aforesaid amino acids in (a)-(c).

23. The isolated AAV particle of claim 22, wherein [E] comprises:
(i) VQN, DQN, VQH, FQN, VQS, VQT, VQP, VRN, VPN, VKN, AQN, VQK, EQN, VQI, LQN, GQT, VLN, VQD, VHN, GQN, VKT, VKK, FQK, VEN, VQY, DKN, GHN, IQN, or VPH, or a substitution, e.g., a conservative substitution, of any of the aforesaid amino acid sequences; or
(ii) VKN, VPN, or VQN.

24. The isolated AAV particle of claim 22 or 23, wherein [A][B][C][D][E] comprises:
(i) the amino acid sequence of any of SEQ ID NOs: 143, 148, 149, 151, 153, 154-158, 160-163, 166, 168, 170, 171, 173-175, 177-179, 181, 182, 184-188, 191-197, 199-210, 212-215, 217-225, 227-231, 233, 234, 236-240, 243-262, 265, 267, 268, 270-277, 279, 282, 284-286, 288-293, 295, 296, 298, 300-314, 316-327, 329, 331, 332, 334, 336, 337-344, 346-350, 352-354, 356-365, 367, 369, 371-380, 382-385, 387, 392-394, 396, 397, 399-401, 404-411, 413-415, 417, 419-429, 432, 433, 435-437, 438, 440-442, 444-447, 450-454, 456, 458-461, 464, 465, 467-469, 471-484, 487-495, 497, 498, 500-503, 505, 507-512, 514-517, 522-525, 528-539, 542-545, 547, 551-555, 558-561, 563-568, 570, 573, 574, 576, 579, 581, 582, 584, 586, 587, 591-596, 598, 601, 604, 605, 606, 607, 610, 612, 614-619, 624-629, 631-636, 640, 641, 645, 646, 649, 650, 656, 658, 661, 663, 664, 666, 668, 669, 670, 672, 673, 674, 675, 677, 679, 683, 684, 686, 688, 689, 691, 693, 695, 696, 697, 699, 700, 701, 702, 704-706, 709-714, 720, 722, 725-731, 733, 736, 740, 745, 749-752, 754, 755, 757, 758, 760-765, 767, 768, 770, 771, 773, 778-780, 783-788, 792-794, 797-799, 801, 802, 804-806, 812, 814, 815, 817, 818, 820, 821, 824, 828, 831, 832, 834-837, 839, 840-845, 847, 848, 850-855, 857-859, 861, 862, 865, 866, 869-872, 874-876, 882-884, 887, 889-895, 897, 899, 901, 903-905, 907, 908, 910, 911, 913, 915, 919, 920, 923, 924, 926, 927, 929, 931-933, 935, 937, 939-949, 952-955, 957, 958, 960, 962, 964, 965, 967, 971, 973, 974, 976, 977, 981, 985-989, 992, 994, 997-1000, 1002, 1004, 1006-1008, 1010, 1013, 1015, 1017, 1018, 1020, 1021, 1023-1025, 1027, 1029-1031, 1033-1035, 1037-1040, 1043, 1046, 1049, 1052, 1053, 1056, 1057, 1059, 1062, 1064, 1065, 1067, 1068, 1070, 1073, 1075, 1077-1080, 1083-1087, 1089, 1090, 1093, 1094, 1097, 1100, 1101, 1103, 1105-1107, 1110-1112, 1114-1117, 1119, 1121, 1125, 1126, 1129, 1132, 1133, 1135;
(ii)
(SEQ ID NO: 566)
PLNGAVHLYAQAQLSPVKN;
(iii)
(SEQ ID NO: 314)
PLNGAVHLYAQAQTGWVPN;
(iv)
(SEQ ID NO: 3648)
PLNGAVHLY;
(v) an amino acid sequence comprising any portion of an amino acid sequence in (i)-(iv), e.g., any 2, 3, 4, 5, 6, 7, 8, 9, 10, 11, 12, 13, 14, 15, 16, 17, or 18 amino acids, e.g., consecutive amino acids, thereof;
(vi) an amino acid sequence comprising at least one, two, or three but no more than four substitutions (e.g., conservative substitutions), insertions, or deletions, relative to any of the amino acid sequences in (i)-(iv); or
(vii) an amino acid sequence comprising at least one, two, or three but no more than four different amino acids, relative to any one of the amino acid sequences in (i)-(iv).

25. The isolated AAV particle of any one of claims 22-24, which comprises from N-terminus to C-terminus [A][B][C][D][E], wherein:
(i) [A] is present immediately subsequent to position 586, and replaces positions 587 and 588 (e.g., A587 and Q588), numbered according to SEQ ID NO: 138;
(ii) [B] is present immediately subsequent to [A];
(iii) [C] is present immediately subsequent to position 588, and replaces positions 589-592 (e.g., A589, Q590, A591, Q592), numbered according to the amino acid sequence of SEQ ID NO: 138;
(iv) [D] is present immediately subsequent to position 592, and replaces positions 593-595 (e.g., T593, G594, W595), numbered according to the amino acid sequence of SEQ ID NO: 138;
(v) [E] is present immediately subsequent to position 595, and replaces positions 596-598 (e.g., V596, Q597, N598), numbered according to the amino acid sequence of SEQ ID NO: 138; and/or
(vi) [A][B][C][D][E] is present immediately subsequent to position 586 and replaces positions 587-598 (e.g., A587, Q588, A589, Q590, A591, Q592, T593, G594, W595, V596, Q597, N598), numbered according to SEQ ID NO: 138.

26. The isolated AAV particle of any one of claims 22-25, wherein:
(i) the AAV capsid variant comprises an amino acid other than A at position 587 and/or an amino acid other than Q at position 588, numbered according to SEQ ID NO: 138;
(ii) [A] corresponds to positions 587-591 of SEQ ID NO: 5, 8, or 3636;
(iii) [B] corresponds to positions 592 to 595 of SEQ ID NO: 5, 8, or 3636;
(iv) [C] corresponds to positions 596-599 of SEQ ID NO: 5, 8, or 3636;
(v) [D] corresponds to positions 600-602 of SEQ ID NO: 5, 8, or 3636;
(vi) [E] corresponds to positions 603-605 of SEQ ID NO: 5, 8, or 3636; and/or
(vii) [A][B][C][D][E] corresponds to positions 587-605 of SEQ ID NO: 5, 8, or 3636.

27. The isolated AAV particle of any one of claims 1-26, which comprises:
(i) the amino acid sequence corresponding to positions 203-743, e.g., a VP3, of any one of SEQ ID NOs: 5, 8, or 3636, or a sequence with at least 90%, 95%, 96%, 97%, 98%, or 99% sequence identity thereto
(ii) the amino acid sequence corresponding to positions 138-743, e.g., a VP2, of any one of SEQ ID NOs: 5, 8, or 3636, or a sequence with at least 90%, 95%, 96%, 97%, 98%, or 99% sequence identity thereto;
(iii) the amino acid sequence of any one of SEQ ID NOs: 5, 8, or 3636 (e.g., a VP1), or an amino acid sequence with at least 90%, 95%, 96%, 97%, 98%, or 99% sequence identity thereto;
(iv) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of any one of SEQ ID NO: 5, 8, or 3636; and/or
(v) an amino acid sequence comprising at least one, two or three, but not more than 30, 20 or 10 different amino acids, relative to the amino acid sequence of any one of SEQ ID NO: 5, 8, or 3636.

28. An isolated, e.g., recombinant, AAV particle comprising an AAV capsid variant and a nucleic acid comprising a transgene encoding encoding a SOD1 targeting polynucleotide wherein.
(A) the SOD1 targeting polynucleotide is a single-stranded antisense RNA molecule, a single-stranded siRNA, or a double-stranded RNA (e.g., a siRNA duplex), which comprises an antisense strand comprising a region that is substantially complementary (e.g., at least 70%, 75%, 80%, 85%, 90%, 95%, 99% or 100% complementary) to at least part of an mRNA transcript of a human SOD1 gene (e.g., a human wild-type SOD gene or a human mutated SOD gene), and wherein the SOD1 targeting polynucleotide inhibits expression of the human SOD1 gene; and
(B) the AAV capsid variant comprises:
(i) the amino acid sequence of PLNGAVHLY (SEQ ID NO: 3648);
(ii) an amino acid sequence comprising at least 5, 6, 7, 8, or 9 consecutive amino acids from the amino acid sequence of PLNGAVHLY (SEQ ID NO: 3648);
(iii) an amino acid sequence comprising at least one, two, or three but no more than four substitutions (e.g., conservative substitutions), relative to the amino acid sequence of PLNGAVHLY (SEQ ID NO: 3648); or
(iv) an amino acid sequence comprising at least one, two, or three but no more than four different amino acids relative to the amino acid sequence of PLNGAVHLY (SEQ ID NO: 3648)
wherein the amino acid sequence of (i)-(iv) is present immediately subsequent to position 586, numbered according to any one of SEQ ID NOs: 5, 8, 138, or 3636;
optionally wherein the AAV capsid variant further comprises:
(a) a VP1 protein comprising the amino acid sequence of SEQ ID NO: 5, 8, 3636;
(b) a VP2 protein comprising the amino acid sequence of positions 138-743 of SEQ ID NO: 5, 8, or 3636;
(c) a VP3 protein comprising the amino acid sequence of positions 203-743 of SEQ ID NO: 5, 8, or 3636; or
(d) an amino acid sequence at least 90% (e.g., at least 92%, 95%, 96%, 97%, 98%, or 99%) identical to any one the amino acid sequences of (a)-(b).

29. The AAV particle of any one of claims 1-28, wherein the SOD1 targeting polynucleotide is a siRNA duplex comprising a sense strand and an antisense strand, wherein the siRNA duplex ranges from 9 to 36 nucleotides in length (e.g., from 15 to 30 nucleotides in length, from 15 to 25 nucleotides in length, from 17 to 22 nucleotides, or from 17-20 nucleotides in length).

30. The AAV particle of claim claim 28 or claim 29, wherein the siRNA duplex comprises:
(i) a sense strand nucleotide sequence comprising at least 15 (e.g., at least 16, 17, 18, 19, 20, 21, 22, or 23) contiguous nucleotides differing by no more than 3 (e.g., by no more than 0, 1 or 2) nucleotides from a sense strand nucleotide sequence comprising any one of SEQ ID NOs: 2004-2172, 2342-2351, 2353-2356, 2358, 2360, 2362, 2364, 2366, 2367, 2370-2380, 2385, 2387, 2389-2394, 2522-2526 and 2528-2534, or a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to any of the aforesaid nucleotide sequences; and
(ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from an antisense sequence comprising any one of SEQ ID NOs: 2173-2341, 2352, 2353, 2357, 2359, 2361, 2363, 2365, 2368, 2384, 2386, 2388, 2395, 2396, 2505-2508, 2510 or 2511, or a nucleotide sequence at least 80%, 81%, 82%, 83%, 84%, 85%, 86%, 87%, 88%, 89%, 90%, 91%, 92%, 93%, 94%, 95%, 96%, 97%, 98% or 99% identical to any of the aforesaid nucleotide sequences.

31. The AAV particle of any one of claims 28-30, wherein the siRNA duplex is selected from:
(i) siRNA duplex ID numbers D-2741 to D2985, and D-4009 to D-4021, or a siRNA duplex at least 80%, 81%, 82%, 83%, 84%, 85%, 86%, 87%, 88%, 89%, 90%, 91%, 92%, 93%, 94%, 95%, 96%, 97%, 98% or 99% identical to any of the aforesaid siRNA duplexes;
(ii) siRNA duplex numbers D-2968, D-2959, or D-4012, or a siRNA duplex at least 80%, 81%, 82%, 83%, 84%, 85%, 86%, 87%, 88%, 89%, 90%, 91%, 92%, 93%, 94%, 95%, 96%, 97%, 98% or 99%, identical to siRNA duplex ID numbers D-2968, D-2959, or D-4012.

32. The AAV particle of any one of claims 28-31, wherein the siRNA duplex comprises:
(i) a sense strand sequence comprising at least 15 (e.g., at least 16, 17, 18, 19, 20, 21, 22, or 23) contiguous nucleotides differing by no more than 3 (e.g., by no more than 0, 1 or 2) nucleotides from SEQ ID NO: 2525, 2376, or 2381; and
(ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from SEQ ID NO: 2507, 2365, or 2363;
(iii) a sense strand sequence comprising SEQ ID NO: 2525, or a nucleotide differing by no more than 3 (e.g., by no more than 0, 1 or 2) nucleotides from SEQ ID NO: 2525, and an antisense strand sequence comprising SEQ ID NO: 2507, or a nucleotide differing by no more than 3 (e.g., by no more than 0, 1 or 2) nucleotides from SEQ ID NO: 2507;
(iv) a sense strand sequence comprising SEQ ID NO: 2376, or a nucleotide differing by no more than 3 (e.g., by no more than 0, 1 or 2) nucleotides from SEQ ID NO: 2376, and an antisense strand sequence comprising SEQ ID NO: 2365, or a nucleotide differing by no more than 3 (e.g., by no more than 0, 1 or 2) nucleotides from SEQ ID NO: 2365; and/or
(v) a sense strand sequence comprising SEQ ID NO: 2381, or a nucleotide differing by no more than 3 (e.g., by no more than 0, 1 or 2) nucleotides from SEQ ID NO: 2381, and an antisense strand sequence comprising SEQ ID NO: 2363, or a nucleotide differing by no more than 3 (e.g., by no more than 0, 1 or 2) nucleotides from SEQ ID NO: 2363.

33. The AAV particle of claim 32, wherein the siRNA duplex comprises:
(i) a sense strand sequence comprising SEQ ID NO: 2525, and an antisense strand sequence comprising SEQ ID NO: 2507;
(ii) a sense strand sequence comprising SEQ ID NO: 2376, and an antisense strand sequence comprising SEQ ID NO: 2365; and/or
(iii) a sense strand sequence comprising SEQ ID NO: 2381, and an antisense strand sequence comprising SEQ ID NO: 2363.

34. The AAV particle of any one of claims 28-33, wherein at least one of the sense strand sequence and the antisense strand sequence comprise a 3′ overhang of at least 1, or 2 nucleotides.

35. The AAV particle of any one of claims 28-34, wherein the siRNA duplex is encoded by a modulatory polynucleotide comprising:
(i) a 5′ flanking region;
(ii) a loop region; and/or
(iii) a 3′ flanking region.

36. The AAV particle of claim 35, wherein the modulatory polynucleotide comprises:
(i) a 5′ flanking region comprising a nucleotide sequence of SEQ ID NO: 2547, 2548, 2549, or 5014, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2547, 2548, 2549, or 5014;
(ii) a loop region comprising a nucleotide sequence of SEQ ID NO: 2550, 2551, 2552, or 2553, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, or four, modifications, but no more than six modifications of SEQ ID NO: 2550, 2551, 2552, or 2553; and/or
(iii) a 3′ flanking region comprising a nucleotide sequence of SEQ ID NO: 2554, 2555, 2556, 2557, or 2558, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2554, 2555, 2556, 2557, or 2558.

37. The AAV particle of claim 28-36, wherein the modulatory polynucleotide comprises:
(i) the 5′ flanking region comprises a nucleotide sequence of SEQ ID NO: 2547;
(ii) the loop region comprises a nucleotide sequence of SEQ ID NO: 2550; and/or
(iii) 3′ flanking region comprises a nucleotide sequence of SEQ ID NO: 2554.

38. The AAV particle of any one of claims 28-35, wherein the siRNA duplex is encoded by
(i) a nucleotide sequence of any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 70% (e.g., at least 75%, 80%, 85%, 90%, 92%, 95%, 97%, 98%, or 99%) identical thereto;
(ii) a nucleotide sequence of any one of SEQ ID NOs: 2562, 2579, or 5022, or a nucleotide sequence at least 70% (e.g., at least 75%, 80%, 85%, 90%, 92%, 95%, 97%, 98%, or 99%) identical thereto; or
(iii) the nucleotide sequence encoding the siRNA duplex comprises the nucleotide sequence of SEQ ID NO: 2562 or a nucleotide sequence at least 70% (e.g., at least 75%, 80%, 85%, 90%, 92%, 95%, 97%, 98%, or 99%) identical thereto.

39. The isolated AAV particle of any one of claims 1-38, which comprises a viral genome comprising a promoter operably linked to the nucleic acid sequence encoding the SOD1 targeting polynucleotide (e.g., a SOD1 targeting RNA agent, e.g., a siRNA duplex).

40. The isolated AAV particle of claim 39, the promoter comprises:
(i) a chicken β-actin (CBA) promoter and/or its derivative CAG, an EF-1a promoter, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof;
(ii) an EF-la promoter variant, e.g., a truncated EF-la promoter, a CBA promoter variant, e.g., a truncated CBA promoter, an insulin promoter variant, e.g., a truncated insulin promoter, or a SYN promoter variant, e.g., a truncated SYN promoter;
(iii) the nucleotide sequence of any one of SEQ ID NOs: 4000-4026, a nucleotide sequence comprising at least one, two, or three but no more than four modifications, e.g., substitutions (e.g., conservative substitutions), insertions, or deletions, relative to the nucleotide sequence of SEQ ID NOs: 4000-4026, or a nucleotide sequence with at least 80% (e.g., 85%, 90%, 95%, 96%, 97%, 98%, or 99%) sequence identity to any one of SEQ ID NOs: 4000-4026; or
(iv) a H1 promoter comprising SEQ ID NO: 128, or a nucleotide sequence with at least 80% (e.g., 85%, 90%, 95%, 96%, 97%, 98%, or 99%) sequence identity to SEQ ID NO: 128.

41. The isolated AAV particle of claim 39 or claim 40, wherein the viral genome further comprises a first exon, a second exon, or both a first exon and second exon, optionally wherein the first and/or second exon comprises the nucleotide sequence of any one of SEQ ID NO: 4045-4048, a nucleotide sequence comprising at least one, two, or three but no more than four modifications, e.g., substitutions, insertions, or deletions, relative to the nucleotide sequence of any one of SEQ ID NO: 4045-4048, or a nucleotide sequence with at least 80% (e.g., 85%, 90%, 95%, 96%, 97%, 98%, or 99%) sequence identity to any one of SEQ ID NO: 4045-4048.

42. The isolated AAV particle of claim 41, wherein the viral genome comprises both a first exon and a second exon, wherein:
(a) the first exon comprises the nucleotide sequence of SEQ ID NO: 4045 or 4046, a nucleotide sequence comprising at least one, two, or three but no more than four modifications, e.g., substitutions, insertions, or deletions, relative to the nucleotide sequence of SEQ ID NO: 4045 or 4046, or a nucleotide sequence with at least 80% (e.g., 85%, 90%, 95%, 96%, 97%, 98%, or 99%) sequence identity to SEQ ID NO: 4045 or 4046;
(b) the second exon comprises the nucleotide sequence of SEQ ID NO: 4047 or 4048, a nucleotide sequence comprising at least one, two, or three but no more than four modifications, e.g., substitutions, insertions, or deletions, relative to the nucleotide sequence of SEQ ID NO: 4047 or 4048, or a nucleotide sequence with at least 80% (e.g., 85%, 90%, 95%, 96%, 97%, 98%, or 99%) sequence identity to SEQ ID NO: 4047 or 4048;
(c) the first exon, second exon, or both are codon optimized; or
(d) the CpG sequences in the first exon, second exon, or both are depleted.

43. The isolated AAV particle of claim 41 or 42, wherein the viral genome comprises a first exon, an intron, and second exon (“exon-intron-exon cassette”), wherein the exon-intron-exon cassette comprises the nucleotide sequence of SEQ ID NO: 4042 or 4043, a nucleotide sequence comprising at least one, two, or three but no more than four modifications, e.g., substitutions, insertions, or deletions, relative to the nucleotide sequence of SEQ ID NO: 4042 or 4043, or a nucleotide sequence with at least 80% (e.g., 85%, 90%, 95%, 96%, 97%, 98%, or 99%) sequence identity to SEQ ID NO: 4042 or 4043, optionally
wherein
(i) the exon-intron-exon cassette is positioned 3′ relative to the promoter, and 5′ relative to the nucleotide sequence encoding the modulatory polynucleotide (e.g., a SOD1 targeting RNA agent, e.g., a siRNA duplex);
(ii) the nucleotide sequence encoding the modulatory polynucleotide (e.g., a SOD1 targeting RNA agent, e.g., a siRNA duplex) is located within (e.g., inserted into) the intron of the exon-intron-exon cassette; or
(iii) the nucleotide sequence encoding the modulatory polynucleotide (e.g., a SOD1 targeting RNA agent, e.g., a siRNA duplex) replaces a portion or all of the intron in the exon-intron-exon cassette.

44. The isolated AAV particle of any claims 39-43, wherein the viral genome further comprises:
(i) an inverted terminal repeat (ITR) sequence, optionally wherein ITR sequence is positioned 5′ relative to the encoded SOD1 targeting polynucleotide, and/or the ITR sequence is positioned 3′ relative to the encoded SOD1 targeting polynucleotide;
(ii) an enhancer;
(iii) a miR binding site;
(iv) a polyadenylation (polyA) signal region;
(v) an intron region;
(vi) an exon region, e.g., at least one, two, or three exon regions; or
(vii) a Kozak sequence.

45. The isolated AAV particle of any one of claims 39-44, wherein the viral genome further comprises a polyA signal region, optionally wherein the polyA signal region comprises SEQ ID NO:
129 or 4027, or a nucleotide sequence with at least 80% (e.g., 85%, 90%, 95%, 96%, 97%, 98%, or 99%) sequence identity to SEQ ID NO: 129 or 4027.

46. The isolated AAV particle of any one of 39-45, wherein the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) an exon, e.g., an exon comprising the nucleotide sequence of any one of SEQ ID NOs: 4045-4048 or variant thereof,
(d) a nucleotide sequence encoding a modulatory polynucleotide (e.g., a SOD1 targeting RNA agent, e.g., a siRNA duplex), e.g., a modulatory polynucleotide comprising the nucleotide sequence of any one of SEQ ID NOs: 2559-2579 and 5022 or variant thereof,
(e) a polyA signal region, e.g., a polyA signal region comprising the nucleotide sequence of SEQ ID NO: 129 or 4027 or variant thereof, and
(f) a 3′ITR, e.g., a 3′ITR comprising the nucleotide sequence of SEQ ID NO: 126 or 130 or variant thereof,
wherein the variant of any of (a)-(f) comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity, relative to the reference sequence, e.g., wherein the reference sequence is a wild-type sequence.

47. The isolated AAV particle of any one of claims 39-45, wherein the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) a first exon, e.g., a first exon comprising the nucleotide sequence of any one of SEQ ID NOs: 4045-4048 or variant thereof,
(d) an intron, e.g., an intron comprising the nucleotide sequence of SEQ ID NO: 4044 or variant thereof,
(e) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
(f) a poly A signal sequence, e.g., a poly A signal sequence comprising the nucleotide sequence of SEQ ID NO: 129 or 4027 or variant thereof, and
(g) a 3′ITR, e.g., a 3′ITR comprising the nucleotide sequence of SEQ ID NO: 126 or 130 or variant thereof,
wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.

48. The isolated AAV particle of any one of claims 39-45, wherein the viral genome comprises, from 5′ to 3′:
(a) a 5′ ITR, e.g., a 5′ITR comprising the nucleotide sequence of SEQ ID NO: 126 or 130 or variant thereof,
(b) a promoter, e.g., a promoter comprising the nucleotide sequence of any one of SEQ ID NOs: 4000-4026 or variant thereof,
(c) a first exon, e.g., a first exon comprising the nucleotide sequence of any one of SEQ ID NOs: 4045-4048 or variant thereof,
(d) an intron, e.g., an intron comprising the nucleotide sequence of SEQ ID NO: 4044 or variant thereof,
(e) a second exon, e.g., a second exon comprising the nucleotide sequence of any one of SEQ ID NOs: 4045-4048 or variant thereof,
(f) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof,
(g) a poly A signal sequence, e.g., a poly A signal sequence comprising the nucleotide sequence of SEQ ID NO: 129 or 4027 or variant thereof, and
(h) a 3′ITR, e.g., a 3′ITR comprising the nucleotide sequence of SEQ ID NO: 126 or 130 or variant thereof,
wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.

49. The isolated AAV particle of any one of claims 39-45, wherein the viral genome comprises the nucleotide sequence of any one of SEQ ID NOs: 4028-4041, a nucleotide sequence differing by no more than 15, 14, 13, 12, 11, 10, 9, 8, 7, 6, 5, 4, 3, 2 or 1 nucleotides, e.g., substitutions, insertions, or deletions, relative to any one of SEQ ID NOs: 4028-4041, or a nucleotide sequence with at least 70%, 75%, 80%, 85%, 90%, 95%, 99% or 100% identity to any one of SEQ ID NOs: 4028-4041.

50. The isolated AAV particle of any one of claims 39-49, wherein the viral genome further comprises a nucleotide sequence encoding a miR binding site, e.g., a miR binding site that modulates, e.g., reduces, expression of the SOD1 targeting polynucleotide encoded by the viral genome in a cell or tissue where the corresponding miRNA is expressed, optionally wherein the encoded miRNA binding site is complementary, e.g., fully complementary or partially complementary, to a miRNA expressed in a cell or tissue of the DRG, liver, heart, hematopoietic, or a combination thereof, and wherein the encoded miR binding site modulates, e.g., reduces, expression of the encoded SOD1 targeting polynucleotide in a cell or tissue of the DRG, liver, heart, hematopoietic lineage, or a combination thereof.

51. The isolated AAV particle of claim 50, wherein the viral genome comprises
(i) at least 1-5 copies of the encoded miR binding site, e.g., at least 1, 2, 3, 4, or 5 copies;
(ii) at least 3 copies of an encoded miR binding sites, optionally wherein all three copies comprise the same miR binding site, or at least one, two, three, or all of the copies comprise a different miR binding site, optionally the at least 3 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.

52. The isolated AAV particle of claim 50 or claim 51, wherein the encoded miR binding site comprises a miR122 binding site, a miR183 binding site, a miR-1 binding site, a miR-142-3p, or a combination thereof, optionally wherein:
(i) the encoded miR122 binding site comprises the nucleotide sequence of SEQ ID NO: 1865, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3672;
(ii) the encoded miR183 binding site comprises the nucleotide sequence of SEQ ID NO: 1847, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 3675;
(iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679; and/or
(iv) the encoded miR-142-3p binding site comprises the nucleotide sequence of SEQ ID NO: 1869, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 1869.

53. The isolated AAV particle of any one of claims 39-52, wherein the viral genome
(i) is single stranded or self-complementary;
(ii) further comprises a nucleotide sequence encoding a Rep protein, e.g., a non-structural protein, wherein the Rep protein comprises a Rep78 protein, a Rep68, Rep52 protein, and/or a Rep40 protein; and/or
(iii) further comprises a nucleic acid encoding a capsid protein, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene.

54. A cell, e.g., a host cell, comprising the AAV particle of any one of claims 1-53.

55. A method of making the AAV particle of any one of claims 1-53, comprising:
(i) providing a host cell comprising a viral genome; and
(ii) incubating the host cell under conditions suitable to enclose the viral genome in an AAV capsid variant, e.g., an AAV capsid variant described herein;
thereby making the AAV particle.

56. A pharmaceutical composition comprising the AAV particle of any one of claims 1-53, and a pharmaceutically acceptable excipient.

57. A method of delivering a SOD1 targeting polynucleotide to a subject, comprising administering an effective amount of the pharmaceutical composition of claim 56, or the AAV particle as described in any one of claims 1-53, thereby delivering the SOD1 targeting polynucleotide to the subject.

58. A method of treating a subject having or diagnosed with having a neurological disorder or a neurodegenerative disorder related to expression of SOD1, comprising administering to the subject an effective amount of the pharmaceutical composition of claim 56, or the AAV of any one claims 1-53.

59. The method of claim 58, wherein the disease or disorder comprises amyotrophic lateral sclerosis (ALS).

60. The method of claim 59, wherein the ALS is:
(i) familial ALS;
(ii) sporadic ALS;
(iii) early stage ALS;
(iv) middle stage ALS; and/or
(v) late stage ALS.

62. The method of claim 60 or claim 61, wherein treatment comprises amelioration of a symptom of ALS in the subject, optionally, wherein the symptom comprises motor neuron degeneration, muscle weakness, stiffness of muscles, muscle atrophy, muscle stiffness, fasciculation development, frontotemporal dementia, slurred speech, difficulty breathing, or a combination thereof.

63. The method of any one of 58-62, wherein the AAV particle is administered to the subject:
(i) intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, via intra-cisterna magna injection (ICM), or via dual ITH and ICM administration; or
(ii) via intravenous administration, optionally wherein the intravenous administration is via focused ultrasound (FUS), e.g., coupled with the intravenous administration of microbubbles (FUS-MB), or MRI-guided FUS coupled with intravenous administration.

64. The method of any one of claims 57-63, wherein the AAV particle is administered to the subject at a dose of:
(a) about 6.7e11 VG/kg to 2e13 VG/kg (e.g., 6.7e11 VG/kg, 2e12 VG/kg, 6.7e12 VG/kg, or 2e13 VG/kg) or about 5e11 VG/kg to 3e13 VG/kg;
(b) about 6.7e10 VG/kg to 6.7e12 VG/kg, about 1.3e11 VG/kg to 3.4e12 VG/kg, or about 2.2e11 VG/kg to 2e12 VG/kg;
(c) about 4e11 VG/kg to 8e11 VG/kg (e.g., about 6.7e11 VG/kg);
(d) about 2e11 VG/kg to 2e13 VG/kg, about 4e11 VG/kg to 1e13 VG/kg, about 6.7e11 VG/kg to about 6e12 VG/kg;
(e) about 1e12 VG/kg to 5e12 VG/kg (e.g., about 2e12 VG/kg);
(f) about 6.7e11 VG/kg to 6.7e13 VG/kg, about 1.3e12 VG/kg to 3.4e13 VG/kg, or about 2.2e12 VG/kg to 2e13 VG/kg;
(g) about 4e12 VG/kg to 8e12 VG/kg (e.g., about 6.7e12 VG/kg);
(h) about 2e12 VG/kg to 2e14 VG/kg, about 4e12 VG/kg to 1e14 VG/kg, about 6.7e12 VG/kg to about 6e13 VG/kg; or
(i) about 1e13 VG/kg to 5e13 VG/kg (e.g., about 2e13 VG/kg).

65. Use of the pharmaceutical composition of claim 56, or the AAV particle of claims 1-53, in the manufacture of a medicament for treating a neurological disorder, a neurodegenerative disorder, a disease associated with SOD1 expression or activity.
